(19) United States
(12) Patent Application Publication
(10) Pub. No.: US 2015/0072021 A1
(43) Pub. Date: Mar. 12, 2015

(54) Methods and Kits for Predicting Outcome and Methods and Kits for Treating Breast Cancer with Radiation Therapy

(71) Applicants: British Columbia Cancer Agency Branch, Vancouver (CA); The University of North Carolina at Chapel Hill, Chapel Hill, NC (US); Washington University, St. Louis, MO (US); University of Utah Research Foundation, Salt Lake City, UT (US)

(72) Inventors: Maggie Chon U. Cheang, Vancouver (CA); Torsten O. Nielsen, North Vancouver (CA); Charles M. Perou, Carrboro, NC (US); Matthew J. Ellis, St. Louis, MO (US); Philip S. Bernard, Salt Lake City, UT (US)

(21) Appl. No.: 14/480,942

(22) Filed: Sep. 9, 2014

Related U.S. Application Data
(60) Provisional application No. 61/875,373, filed on Sep. 9, 2013.
(60) Provisional application No. 61/990,948, filed on May 9, 2014.

(51) Int. Cl. C12Q 1/68; G01N 33/574

(52) U.S. Cl. CPC C12Q 1/6886; G01N 33/57415; C12Q 2600/158; C12Q 2600/118
USPC 424/649; 435/7.1; 435/6.11

(57) ABSTRACT

The application describes methods and kits for screening subjects with breast cancer to determine if the breast cancer will be responsive to a post-mastectomy breast cancer therapy including radiation. The application further describes methods and kits for treating subjects with post-mastectomy breast cancer by screening them for the likelihood of the effectiveness of treating the cancer with a therapy including radiation and administering the therapy in subjects when it is found that radiation is likely to be effective.

### CROSS-REFERENCE TO RELATED APPLICATIONS

This application claims priority to U.S. Provisional Patent Application Ser. No. 61/875,373 filed Sep. 9, 2013 and to U.S. Provisional Patent Application Ser. No. 61/990,948 filed May 9, 2014, the contents of which are herein incorporated by reference in their entirety.

### FIELD OF THE INVENTION

This disclosure relates generally to the field of cancer biology, and specifically, to the fields of detection and identification of specific cancer cell phenotypes and correlation with appropriate therapies.

### INCORPORATION BY REFERENCE OF SEQUENCE LISTING

The contents of the text file named “NATE-022001US_ST25.txt”, which was created on Sep. 8, 2014 and is 328,667 bytes in size, are hereby incorporated by reference in their entireties.

### BACKGROUND OF THE INVENTION

Radiation therapy (also known as radiotherapy or radiation oncology) is often utilized following lumpectomy or mastectomy to reduce or control malignant cancer cells that remain post-surgery, i.e., as an adjuvant therapy, and is known to lower the chances of breast cancer recurrence and breast cancer death. Radiation is used after mastectomy to treat the chest wall and the lymph nodes around the collarbone and axillary nodes in the underarm area. However, there are various adverse side effects associated with radiation therapy, such as nausea and vomiting, intestinal discomfort, mouth, throat and stomach sores, damage to epithelial surfaces, edema, infertility, fibrosis, lymphedema, hypopituitarism and epilation. Thus, there is a need in the art to determine types of cancer and identifying subjects having such cancer types that respond best to radiation-based therapy and which types of cancer and subjects having such cancer types would be better treated with non-radiation-based therapy; accordingly, an optimal treatment is provided to the subject in need thereof. The present invention addresses these needs.

### SUMMARY OF THE INVENTION

The present invention provides a method of predicting local-regional relapse free, or breast cancer specific survival in a subject having a breast cancer including steps of: (a) obtaining a biological sample from the subject and (b) assaying the biological sample to determine whether the biological sample is classified as a Luminal A subtype, Luminal B subtype, Basal-like subtype, or HER2-enriched subtype, wherein the subtypes are determined using a measurement of at least 10, at least 15, at least 20, at least 25, at least 40, 41, 42, 43, 44, 45, 46, 47, 48, 49 or all 50 of the genes listed in Table 1, wherein (1) if the biological sample is classified as a Luminal A subtype or Basal-like subtype, a post-mastectomy breast cancer treatment including radiation is more likely to prolong local-regional relapse free survival or breast cancer specific survival of the subject or (2) if the biological sample is classified as a Luminal B subtype or HER2-enriched subtype, a post-mastectomy breast cancer treatment including radiation is not likely to prolong local-regional relapse free survival or breast cancer specific survival of the subject.

The present invention also provides a method of screening for the likelihood of the effectiveness of a post-mastectomy breast cancer treatment including radiation in a subject in need thereof including steps of: (a) obtaining a biological sample from the subject and (b) assaying the biological sample to determine whether the biological sample is classified as a Luminal A, Luminal B, HER2-enriched, or Basal-like subtype, wherein the subtype is determined using a measurement of at least 10, at least 15, at least 20, at least 25, at least 40, 41, 42, 43, 44, 45, 46, 47, 48, 49 or all 50 of the genes listed in Table 1, wherein (1) if the biological sample is classified as a Luminal A subtype or Basal-like subtype, the post-mastectomy breast cancer treatment including radiation is more likely to be effective in the subject or (2) if the biological sample is classified as a Luminal B subtype or HER2-enriched subtype, the post-mastectomy breast cancer treatment including radiation is not likely to be effective in the subject.

The present invention also provides a method of treating breast cancer in a subject in need thereof including steps of: (a) obtaining a biological sample from the subject, (b) assaying the biological sample to determine whether the biological sample is classified as a Luminal A, Luminal B, HER2-enriched, or Basal-like subtype, wherein the subtype is determined using a measurement of at least 10, at least 15, at least 20, at least 25, at least 40, 41, 42, 43, 44, 45, 46, 47, 48, 49 or all 50 of the genes listed in Table 1, and (c) administering a breast cancer treatment to the subject, wherein (1) if the biological sample is classified as a Luminal A or Basal-like subtype, the subject is administered a post-mastectomy breast cancer treatment including radiation or (2) if the biological sample is a Luminal B or HER2-enriched subtype, the subject is administered a breast cancer treatment not including radiation, thereby treating breast cancer in the subject.

In any of the above methods, preferably, the subtypes are determined using expression levels (e.g., RNA expression levels) of at least 40 of the genes listed in Table 1, e.g., 46 or 50 of the genes listed in Table 1. The step of assaying may include detecting expression levels of at the least the following 24 genes from the at least 40 of the genes listed in Table 1, i.e., FOXA1, MLPH, ESR1, FOXC1, CDC20, ANLN, MAPT, ORC6L, CEP55, MKI67, UBE2C, KNTC2, EXO1, PTTG1, MELK, BIRC5, GPR160, RRM2, SRFP1, NAT1, KIF2C, CXXC5, MIA and BCL2. Expression levels of CCNE1, CDC6, CDCA1, CENPF, TYMS, and UBE2T may additionally be detected. In embodiments, expression level of each gene in the NANO46 gene set (which is all 50 genes in Table 1 with the exception of MYBL2, BIRC5, GRB7 and CCNB1) is detected. Additionally, expression levels of housekeeping genes may be detected. Expression levels of the at least 40 genes as well as a plurality of (e.g., eight or more) housekeeping genes can be detected in a single hybridization reaction. Expression levels of the at least 40 genes may be normalized to expression levels of the plurality of housekeeping genes. To control for any differences in the intact RNA amount in the reference sample, the levels of the at least 40 genes are normalized against the mean of the level of plurality of housekeeping genes.

A synthetic RNA reference sample, comprising in vitro transcribed RNA targets from the at least 40 genes and the plurality of housekeeping genes, may be assayed and used as a control. Further, to control for any variation in the assay procedure, the above normalized expression levels for each of the at least 40 genes from a biological sample are then further normalized to the normalized levels from each of the at least 40 genes of the synthetic reference sample. The normalized gene expression levels are then log transformed and scaled using two scaling factors.

The step of assaying may include one or more steps of generating a gene expression profile based on expression of the genes in the biological sample, comparing the gene expression profile for the biological sample to centroids constructed from gene expression data for the at least 40 of the genes listed in Table 1 for the Luminal A, Luminal B, HER2-enriched or Basal-like subtypes, utilizing a supervised algorithm and calculating the distance of the gene expression profile for the biological sample to each of the centroids, and classifying the biological sample as a Luminal A, Luminal B, HER2-enriched or Basal-like subtype based upon the nearest centroid. More specifically, a computational algorithm based on a Pearson's correlation compares the normalized and scaled gene expression profile of the entirety of the at least 40 genes from the biological sample to prototypical expression signatures (termed “centroids”) which define each of the four breast cancer intrinsic subtypes, e.g., derived from gene expression data deposited with the National Center for Biotechnology Information Gene Expression Omnibus (GEO) (as examples, with accession number GSE2845 or GSE10886). The Pearson's correlation calculation assigns the patient breast tumor sample to the intrinsic subtype with the most similar expression profile or centroid score across the at least 40 genes. The Pearson's correlation of the totality of the at least 40 genes to the four centroids results in four numerical values that each range from −1 to +1 where a value of +1 is a perfectly correlated expression profile, −1 is a perfectly anti-correlated profile and 0 is completely uncorrelated. Features of the above-mentioned steps are included in the “PAM50 classification model” or the “NANO46 classification model”, as described below.

At least one of the above described steps is performed on a computer or electronic computational device.

In embodiments, assaying includes detecting expression levels of HER2.

The breast cancer can be primary breast cancer, locally advanced breast cancer or metastatic breast cancer. The subject can be a mammal. Preferably, the subject is human. The subject may be a male or a female. The subject has been diagnosed by a skilled artisan as having a breast cancer and is included in a subpopulation of humans who currently have breast cancer or had breast cancer. The subject that has breast cancer can be pre-mastectomy or post-mastectomy. Preferably the subject is post-mastectomy. The subject may have undergone breast-conserving therapy. The subject that has breast cancer may have been previously been treated with an anti-cancer or chemotherapeutic agent. Preferably the subject has not been previously treated with an anti-cancer agent or chemotherapeutic agent. The subject may have been previously been treated with radiation. Preferably the subject has not been previously treated with radiation. The subject can be pre-menopausal or post-menopausal. Preferably, the subject is pre-menopausal. The subject can have node-positive breast cancer. Preferably, the subject has node-positive breast cancer. The subject can have estrogen receptor positive or estrogen receptor negative breast cancer. The subject that has estrogen receptor positive breast cancer may also undergo or be subjected to oophorectomy, alone or in addition to other breast cancer treatments. The subject may have Stage I or II, lymph node-negative, breast cancer or Stage II, lymph node positive, breast cancer.

The breast cancer treatment that includes radiation can also include one or more anti-cancer or chemotherapeutic agents. Classes of anti-cancer or chemotherapeutic agents can include anthracycline agents, alkylating agents, nucleoside analogs, platinum agents, taxanes, vinca agents, anti-estrogen drugs, aromatase inhibitors, ovarian suppression agents, endocrine/hormonal agents, bisphophonate therapy agents and targeted biological therapy agents. Specific anti-cancer or chemotherapeutic agents include cyclophosphamide, fluorouracil (or 5-fluorouracil or 5-FU), methotrexate, thiotepa, carboplatin, cisplatin, gemcitabine, anthracycline, taxanes, paclitaxel, protein-bound paclitaxel, docetaxel, vinorelbine, tamoxifen, raloxifene, toremifene, fulvestrant, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb or bevacizumab, or combinations thereof. Preferably, the treatment that includes radiation also includes cyclophosphamide, fluorouracil (or 5-fluorouracil or 5-FU), methotrexate, or combinations thereof one such combination is CMF which includes cyclophosphamide, methotrexate, and fluorouracil.

The assaying of the biological sample to determine whether the biological sample is classified as either a Luminal A, Luminal B, HER2-enriched, or Basal-like subtype cancer is performed using RNA expression profiling, immunohistochemistry (IHC) or fluorescence in situ hybridization (FISH). Preferably, the assay is RNA expression profiling. The expression of the members of the gene list of Table 1 can be determined using a nanoreporter and the nanoreporter code system (nCounter® Analysis system; NanoString Technologies, Seattle, Wash.). Preferably, expression of the members of the gene list of Table 1 can be determined using a reporter probe and capture probe for the detection of at least 10, at least 15, at least 20, at least 25, at least 40, 41, 42, 43, 44, 45, 46, 47, 48, 49 or all 50 of the genes listed in Table 1. In particular, expression of the “NANO46” set of genes is determined (which is by determining the expression of all 50 genes in Table 1 with the exception of determining the expression of MYBL2, BIRC5, GRB7 and CCNB1). Preferably, there is only one reporter probe/capture probe pair for any one gene of Table 1 to be detected.

The biological sample can be a cell, a tissue or a bodily fluid. The tissue can be sampled from a biopsy or smear. The biological sample can be a tumor. The tumor can be an estrogen receptor positive tumor or an estrogen receptor negative tumor. The sample can also be a sampling of bodily fluids. The bodily fluid can include blood, lymph, urine, saliva, nipple aspirates and gynecological fluids. The biological sample can be a formalin fixed paraffin embedded tissues (FFPE) sample.

When a biological sample is classified as either a Luminal A, Luminal B, HER2-enriched, or Basal-like subtype cancer, the subject from which the biological sample is obtained is classified as having, respectively, a Luminal A, Luminal B, HER2-enriched, or Basal-like subtype cancer. A subject is assigned to a recommended treatment group based on his/her classified cancer subtype. Finally, a recommend treatment to be provided to a subject depends on the group to which the subject is assigned.

In embodiments, a computational algorithm then calculates a Risk of Recurrence (ROR) score. In embodiments, the ROR score is calculated using coefficients from a Cox model that includes (1) Pearson's correlation of the expression profiles of the at least 40 genes (e.g., the NANO46 gene set) in the biological sample with the expected profiles for the four intrinsic subtypes (as described above), (2) a proliferation score (determined from the mean gene expression of a subset of 18 proliferation genes of the at least 40 genes (as described below) and (3) gross tumor size of the subject's tumor. The variables are multiplied by the corresponding coefficients from the Cox Model to generate the score, which is then adjusted to a 0-100 scale. The 0-100 ROR score is correlated with the probability of distant recurrence at ten years (Distant Recurrence-Free Survival (DRFS) at 10 years). Risk categories (low, intermediate, or high) are also calculated based on cut-offs for risk of recurrence score determined in a clinical validation study.

In embodiments, a risk of recurrence (ROR) score of 0 to 40 is a low risk of recurrence for a node-negative cancer, a ROR score of 0 to 15 is a low risk of recurrence for a node-positive cancer, a ROR score of 61 to 100 is a high risk of recurrence for a node-negative cancer, and a ROR score of 41 to 100 is a high risk of recurrence for a node-positive cancer.

As used herein a ROR score can be calculated using any method or formula known in the art. Exemplary formulae include Equations 1 to 6, as described herein.

The at least 40 genes set contains many genes that are known markers for proliferation. The methods and kits of the present invention provide for the determination of subsets of genes that provide a proliferation signature. The methods and kits of the present invention can include steps and reagents for determining the expression of at least one of, a combination of, or each of, a 18-gene subset of the intrinsic genes of Table 1 selected from ANLN, CCNE1, CDC20, CDC6, CDCA1, CENPF, CEP55, EXO1, KIF2C, KNTC2, MELK, MKI67, ORC6L, PTTG1, RRM2, TYMS, UBE2C and/or UBE2T. Preferably, the expression of each of the 18-gene subset of the gene set of Table 1 is determined to provide a proliferation score. The expression of one or more of these genes may be determined and a proliferation signature index can be generated by averaging the normalized expression estimates of one or more of these genes in a sample. The sample can be assigned a high proliferation signature, a moderate/intermediate proliferation signature, a low proliferation signature or an ultra-low proliferation signature. Methods of determining a proliferation signature from a biological sample are as described in Nielsen et al. Clin. Cancer Res., 16(21):5222-5232 (2009) and supplemental online material.

The present invention provides a kit for predicting local-regional relapse free or breast cancer specific survival in a subject having a breast cancer including reagents (e.g., sets of reporter/capture probes and/or primers) sufficient for detecting expression of at least 10, at least 15, at least 20, at least 25, at least 40, 41, 42, 43, 44, 45, 46, 47, 48, 49 or all 50 of the genes listed in Table 1; instructions for performing an assay to classify a biological sample from the subject as a Luminal A, Luminal B, HER2-enriched, or Basal-like subtype, by using the reagents to detect or measure expression of at least 10, at least 15, at least 20, at least 25, at least 40, 41, 42, 43, 44, 45, 46, 47, 48, 49 or all 50 of the genes listed in Table 1; instructions providing information allowing a user to classify whether the biological sample from the subject is a Luminal A, Luminal B, HER2-enriched, or Basal-like subtype by using the reagents to detect or measure expression at least 10, at least 15, at least 20, at least 25, at least 40, 41, 42, 43, 44, 45, 46, 47, 48, 49 or all 50 of the genes listed in Table 1; and instructions for obtaining a prediction whether a treatment including radiation is more likely or not likely to prolong local-regional relapse free or breast cancer specific survival in the subject based on the classified cancer subtype, wherein (a) if the biological sample is classified as a Luminal A subtype or Basal-like, a post-mastectomy breast cancer treatment including radiation is more likely to prolong local-regional relapse free survival or breast cancer specific survival of the subject and (b) if the biological sample is classified as a Luminal B or HER2-enriched subtype, a post-mastectomy breast cancer treatment including radiation is not likely to prolong local-regional relapse free survival or breast cancer specific survival of the subject. The instructions may provide a recommended treatment for the subject based on the obtained prediction. The instructions may further specify how to determine a proliferation score/signature, how to utilize clinicopathological variables in calculations, and how to calculate risk of recurrence (ROR) scores/signatures, e.g., which may be based in part of expression data of the NANO46 set of genes. The kit may also contain reagents sufficient to facilitate detection and/or quantitation of HER2, in order to classify cells as HER2+. The kit may include a positive and/or negative control reference sample(s). The kit may include reagents for detecting expression of one or more housekeeping genes, DNA Repair genes, and/or tumor suppressor genes (e.g., RB1). The kit may further comprise a non-transitory computer readable medium including, at least, any of the above-described instructions. The kit may comprise an array. The kit may include reagents and instructions for determining a VEGF-signature score (as described below, including Table 7).

The present invention also provides a kit for screening for the likelihood of the effectiveness of a post-mastectomy breast cancer treatment including radiation in a subject in need thereof including reagents (e.g., sets of reporter/capture probes and/or primers) sufficient for detecting expression of at least 10, at least 15, at least 20, at least 25, at least 40, 41, 42, 43, 44, 45, 46, 47, 48, 49 or all 50 of the genes listed in Table 1; instructions for performing an assay to classify a biological sample from the subject as a Luminal A, Luminal B, HER2-enriched or Basal-like subtype, by using the reagents to detect or measure expression of at least 10, at least 15, at least 20, at least 25, at least 40, 41, 42, 43, 44, 45, 46, 47, 48, 49 or all 50 of the genes listed in Table 1; instructions providing information allowing a user to classify whether the biological sample from the subject is a Luminal A, Luminal B, HER2-enriched, or Basal-like subtype by using the reagents to detect or measure expression of at least 10, at least 15, at least 20, at least 25, at least 40, 41, 42, 43, 44, 45, 46, 47, 48, 49 or all 50 of the genes listed in Table 1; and instructions for determining the likelihood of the effectiveness of a post-mastectomy breast cancer treatment including radiation in the subject based on the classified cancer subtype, wherein (a) if the biological sample is classified as a Luminal A or Basal-like subtype, a post-mastectomy breast cancer treatment including radiation is more likely to be effective in the subject or (b) if the biological sample is classified as a Luminal B or HER2-enriched subtype, a post-mastectomy breast cancer treatment including radiation is not likely to be effective in the subject. The instructions provide a recommended treatment based on the determined likelihood of effectiveness. The instructions may further specify how to determine a proliferation score/signature, how to utilize clinicopathological variables in calculations, and how to calculate risk of recurrence (ROR) scores/signatures, e.g., which may be based in part of expression data of the NANO46 set of genes. The kit may also contain reagents sufficient to facilitate detection and/or quantitation of HER2, in order to classify cells as HER2+. The kit may include a positive and/or negative control reference sample(s). The kit may include reagents for detecting expression of one or more housekeeping genes, DNA Repair genes, and/or tumor suppressor genes (e.g., RB1). The kit may further comprise a non-transitory computer readable medium including, at least, any of the above-described instructions. The kit may comprise an array. The kit may include reagents and instructions for determining a VEGF-signature score.

The present invention also provides a kit for treating breast cancer in a subject in need thereof including reagents (e.g., sets of reporter/capture probes and/or primers) sufficient for detecting expression of at least 10, at least 15, at least 20, at least 25, at least 40, 41, 42, 43, 44, 45, 46, 47, 48, 49 or all 50 of the genes listed in Table 1; instructions for performing an assay to classify a biological sample from the subject as a Luminal A, Luminal B, HER2-enriched or Basal-like subtype, by using the reagents to detect or measure expression of at least 10, at least 15, at least 20, at least 25, at least 40, 41, 42, 43, 44, 45, 46, 47, 48, 49 or all 50 of the genes listed in Table 1; instructions providing information allowing a user to classify whether the biological sample from the subject is a Luminal A, Luminal B, HER2-enriched, or Basal-like subtype by using the reagents to measure at least 10, at least 15, at least 20, at least 25, at least 40, 41, 42, 43, 44, 45, 46, 47, 48, 49 or all 50 of the genes listed in Table 1; and instructions for administering a post-mastectomy breast cancer treatment including radiation if the biological sample is classified as a Luminal A or Basal-like subtype and instructions for administering a post-mastectomy breast cancer treatment not including radiation if the biological sample is classified as a Luminal B or HER2-enriched subtype. The instructions may further specify how to determine a proliferation score/signature, how to utilize clinicopathological variables in calculations, and how to calculate risk of recurrence (ROR) scores/signatures, e.g., which may be based in part of expression data of the NANO46 set of genes. The kit may also contain reagents sufficient to facilitate detection and/or quantitation of HER2, in order to classify cells as HER2+. The kit may include a positive and/or negative control reference sample(s). The kit may include reagents for detecting expression of one or more housekeeping genes, DNA Repair genes, and/or tumor suppressor genes (e.g., RB1). The kit may further comprise a non-transitory computer readable medium including, at least, any of the above-described instructions. The kit may comprise an array. The kit may include reagents and instructions for determining a VEGF-signature score.

Preferably, the kit provides reagents sufficient for the detection of at least 40 of the genes listed in Table 1. Preferably, the kit provides reagents sufficient for the detection of at least 45 of the genes listed in Table 1, i.e., 46 of the genes listed in Table 1. The reagents sufficient for the detection of the at least 10, at least 15, at least 20, at least 25, at least 40, 41, 42, 43, 44, 45, 46, 47, 48, 49 or all 50 of the genes listed in Table 1 can include an array (e.g., a microarray) or a microfluidic device. Preferably, the reagents include a reporter probe and capture probe for the detection of at least 10, at least 15, at least 20, at least 25, at least 40, 41, 42, 43, 44, 45, 46, 47, 48, 49 or all 50 of the genes listed in Table 1. Preferably, the kit includes reagents sufficient to detect one or more housekeeping genes, DNA Repair genes, and/or tumor suppressor genes (e.g., RB1). Preferably, there is only one reporter probe/capture probe pair for any one gene of Table 1 to be detected or only one housekeeping gene. Preferably, the kit includes reagents sufficient to facilitate detection and/or quantitation of HER2. Preferably, the kit includes reagents sufficient to determine a VEGF-signature score. Preferably, the kit includes instructions for utilizing the reagents and for performing any of the methods provided in the instant invention.

The term “likely” as used herein has the meaning commonly understood by a person skilled in the art to which this invention belongs. For example, if a subject is “more likely” to benefit from a therapy, it would be recommended for a health care provider to select the therapy for the subject.

The term “measurement” as used herein includes obtaining, measuring, or detecting a numeric value of a quantifiable property, e.g., expression level of a gene, and also includes calculations using the value, e.g., the deviation of a gene's expression level in a test sample relative to a control sample, a correlation, and a statistic.

Any of the above aspects and embodiments can be combined with any other aspect or embodiment.

Unless otherwise defined, all technical and scientific terms used herein have the same meaning as commonly understood by one of ordinary skill in the art to which this invention belongs. In the specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In the case of conflict, the present specification, including definitions, will control. In addition, the materials, methods, and examples are illustrative only and are not intended to be limiting. Other features and advantages of the invention will be apparent from the following detailed description and claim.

### BRIEF DESCRIPTION OF THE DRAWINGS

The above and further features will be more clearly appreciated from the following detailed description when taken in conjunction with the accompanying drawings.

FIGS. 1A and 1B show loco-regional relapse and breast cancer specific survival (BCSS), respectively, for subjects whose tumor samples are classified as Luminal A, with or without radiation therapy.

FIGS. 2A and 2B show loco-regional free survival and BCSS, respectively, for subjects whose tumor samples are classified as Luminal B, with or without radiation therapy.

FIGS. 3A and 3B show loco-regional free survival and BCSS, respectively, for subjects whose tumor samples are classified as HER2-enriched, with or without radiation therapy.

FIGS. 4A and 4B show loco-regional free survival and BCSS, respectively, for subjects whose tumor samples are classified as Basal-like, with or without radiation therapy.

FIG. 5 shows 10-year BCSS for subpopulations of Basal-like tumors, with or without radiation therapy.

FIGS. 6A and 6B show loco-regional free survival and BCSS, respectively, for subjects who are classified as low risk based on their Risk of Recurrence Score (subtypes centroid based), ROR-S, with or without radiation therapy.

FIGS. 7A and 7B show loco-regional free survival and BCSS, respectively, for subjects who are classified as moderate/intermediate risk based on their Risk of Recurrence Score (subtypes centroid based), ROR-S, with or without radiation therapy.

FIGS. 8A and 8B show loco-regional free survival and BCSS, respectively, for subjects who are classified as high risk based on their Risk of Recurrence Score (subtypes centroid based), ROR-S, with or without radiation therapy.

FIG. 9 is a schematic of the Breast Cancer Intrinsic Subtyping test.

FIG. 10 is a schematic of an algorithm process.

### DETAILED DESCRIPTION OF THE INVENTION

The present invention provides a method of determining whether a post-mastectomy breast cancer treatment comprising radiation is optimal for administration to a patient suffering from breast cancer. Determining whether a breast cancer patient should receive a treatment including radiation includes classifying the subtype of the breast cancer using a gene expression set. The disclosure also provides a method of treating breast cancer by determining whether a post-mastectomy breast cancer patient should receive a treatment including radiation and then administering the optimal breast cancer treatment to the patient based on that determination.

Intrinsic genes are statistically selected to have low variation in expression between biological sample replicates from the same individual and high variation in expression across samples from different individuals. Thus, intrinsic genes are used as classifier genes for breast cancer classification. Although clinical information was not used to derive the breast cancer intrinsic subtypes, this classification has proved to have prognostic significance. Intrinsic gene screening can be used to classify breast cancers into various subtypes. The major intrinsic subtypes of breast cancer are referred to as Luminal A (LumA), Luminal B (LumB), HER2-enriched (Her-2-E), Basal-like, and Normal-like (Perou et al. Nature, 406(6797):747-52 (2000); Sorlie et al. PNAS, 98(19):10869-74 (2001)).

The PAM50 gene expression assay, as described herein, is able to identify intrinsic subtype from standard formalin fixed paraffin embedded tumor tissue (also see, Parker et al. J Clin Oncol., 27(8):1160-7 (2009) and U.S. Patent Application Publication No. 2011/0145176). The methods utilize a supervised algorithm to classify subject samples according to breast cancer intrinsic subtype. This algorithm, referred to herein as the “PAM50 classification model”, is based on the gene expression profile of a defined subset of intrinsic genes that has been identified herein as superior for classifying breast cancer intrinsic subtypes. See, U.S. Patent Application Publication No. 2011/0145176. The subset of genes, along with exemplary primers specific for their detection, is provided in Table 1. The subset of genes, along with exemplary probes specific for their detection, is provided in Table 2. The exemplary primers and target specific probe sequences are merely representative and not meant to limit the invention. The skilled artisan can utilize any primer and/or target sequence-specific probe for detecting any of (or each of) the genes in Table 1.

TABLE 1
PAM50 Intrinsic Gene List
Table 1

| | REPRESENTATIVE | | | | |
|---|---|---|---|---|---|
| | GENBANK | | SEQ | | SEQ |
| GENE | ACCESSION | | ID | | ID |
| NAME | NUMBER | FORWARD PRIMER | NO: | REVERSE PRIMER | NO: |
| ACTR3B | NM_020445 | AAAGATTCCTGGGA | 1 | TGGGGCAGTTCTGTA | 51 |
| | NM_001040135 | CCTGA | | TTACTTC | |
| ANLN | NM_018685 | ACAGCCACTTTCAG | 2 | CGATGGTTTTGTACA | 52 |
| | | AAGCAAG | | AGATTTCTC | |
| BAG1 | NM_004323 | CTGGAAGAGTTGAA | 3 | GCAAATCCTTGGGC | 53 |
| | | TAAAGAGC | | AGA | |
| BCL2 | NM_000633 | TACCTGAACCGGCA | 4 | GCCGTACAGTTCCAC | 54 |
| | | CCTG | | AAAGG | |
| BIRC5 | NM_001012271 | GCACAAAGCCATTC | 5 | GACGCTTCCTATCAC | 55 |
| | | TAAGTC | | TCTATTC | |
| BLVRA | BX647539 | GCTGGCTGAGCAGA | 6 | TTCCTCCATCAAGAG | 56 |
| | | AAG | | TTCAACA | |
| CCNB1 | NM_031966 | CTTTCGCCTGAGCCT | 7 | GGGCACATCCAGAT | 57 |
| | | ATTT | | GTTT | |
| CCNE1 | BC035498 | GGCCAAAATCGACA | 8 | GGGTCTGCACAGAC | 58 |
| | | GGAC | | TGCAT | |
| CDC20 | BG256659 | CTGTCTGAGTGCCG | 9 | TCCTTGTAATGGGGA | 59 |
| | | TGGAT | | GACCA | |
| CDC6 | NM_001254 | GTAAATCACCTTCT | 10 | ACTTGGGATATGTGA | 60 |
| | | GAGCCT | | ATAAGACC | |
| CDCA1 | NM_031423 | GGAGGCGGAAGAA | 11 | GGGGAAAGACAAAG | 61 |
| | | ACCAG | | TTTCCA | |
| CDH3 | BC041846 | GACAAGGAGAATCA | 12 | ACTGTCTGGGTCCAT | 62 |
| | | AAAGATCAGC | | GGCTA | |
| CENPF | NM_016343 | GTGGCAGCAGATCA | 13 | GGATTTCGTGGTGGG | 63 |
| | | CAA | | TTC | |
| CEP55 | AB091343 | CCTCACGAATTGCT | 14 | CCACAGTCTGTGATA | 64 |
| | | GAACTT | | AACGG | |
| CXXC5 | BC006428 | CATGAAATAGTGCA | 15 | CCATCAACATTCTCT | 65 |
| | | TAGTTTGCC | | TTATGAACG | |
| EGFR | NM_005228 | ACACAGAATCTATA | 16 | ATCAACTCCCAAAC | 66 |
| | | CCCACCAGAGT | | GGTCAC | |
| ERBB2 | NM_001005862 | GCTGGCTCTCACAC | 17 | GCCCTTACACATCGG | 67 |
| | | TGATAG | | AGAAC | |
| ESR1 | NM_001122742 | GCAGGGAGAGGAGT | 18 | GACTTCAGGGTGCTG | 68 |
| | | TTGT | | GAC | |
| EXO1 | NM_130398 | CCCATCCATGTGAG | 19 | TGTGAAGCCAGCAA | 69 |
| | | GAAGTATAA | | TATGTATC | |
| FGFR4 | AB209631 | CTTCTTGGACCTTGG | 20 | TATTGGGAGGCAGG | 70 |
| | | CG | | AGGTTTA | |
| FOXA1 | NM_004496 | GCTACTACGCAGAC | 21 | CTGAGTTCATGTTGC | 71 |
| | | ACG | | TGACC | |
| FOXC1 | NM_001453 | GATGTTCGAGTCAC | 22 | GACAGCTACTATTCC | 72 |
| | | AGAGG | | CGTT | |
| GPR160 | AJ249248 | TTCGGCTGGAAGGA | 23 | TATGTGAGTAAGCTC | 73 |
| | | ACC | | GGAGAC | |
| GRB7 | NM_005310 | CGTGGCAGATGTGA | 24 | AGTGGGCATCCCGT | 74 |
| | | ACGA | | AGA | |
| HSPC150 | NM_014176 | GGAGATCCGTCAAC | 25 | AGTGGACATGCGAG | 75 |
| (UBE2T) | | TCCAAA | | TGGAG | |
| KIF2C | NM_006845 | TGGGTCGTGTCAGG | 26 | CACCGCTGGAAACT | 76 |
| | | AAAC | | GAAC | |
| KNTC2 | NM_006101 | CGCAGTCATCCAGA | 27 | CGTGCACATCCATGA | 77 |
| | | GATGTG | | CCTT | |
| KRT14 | BC042437 | ACTCAGTACAAGAA | 28 | GAGGAGATGACCTT | 78 |
| | | AGAACCG | | GCC | |
| KRT17 | AK095281 | GTTGGACCAGTCAA | 29 | GCCATAGCCACTGCC | 79 |
| | | CATCTCTG | | ACT | |
| KRT5 | M21389 | TGTGGCTCATTAGG | 30 | CTTCGACTGGACTCT | 80 |
| | | CAAC | | GT | |
| MAPT | NM_001123066 | GACTCCAAGCGCGA | 31 | CAGACATGTTGGTAT | 81 |
| | | AAAC | | TGCACATT | |
| MDM2 | M92424 | CCACAAAATATTCA | 32 | AGGCGATCCTGGGA | 82 |
| | | TGGTTCTTG | | AATTAT | |
| MELK | NM_014791 | CCAGTAGCATTGTC | 33 | CCCATTTGTCTGTCT | 83 |
| | | CGAG | | TCAC | |
| MIA | BG765502 | GTCTCTGGTAATGC | 34 | CTGATGGTTGAGGCT | 84 |
| | | ACACT | | GTT | |
| MK167 | NM_002417 | GTGGAATGCCTGCT | 35 | CGCACTCCAGCACCT | 85 |
| | | GACC | | AGAC | |
| MLPH | NM_024101 | AGGGGTGCCCTCTG | 36 | TCACAGGGTCAAAC | 86 |
| | | AGAT | | TTCCAGT | |
| MMP11 | NM_005940 | CGAGATCGCCAAGA | 37 | GATGGTAGAGTTCC | 87 |
| | | TGTT | | AGTGATT | |
| MYBL2 | BX647151 | AGGCGAACACACAA | 38 | TCTGGTCACGCAGG | 88 |
| | | CGTC | | GCAA | |
| MYC | NM_002467 | AGCCTCGAACAATT | 39 | ACACAGATGATGGA | 89 |
| | | GAAGA | | GATGTC | |
| NAT1 | BC013732 | ATCGACTGTGTAAA | 40 | AGTAGCTACATCTCC | 90 |
| | | CAACTAGAGAAGA | | AGGTTCTCTG | |
| ORC6L | NM_014321 | TTTAAGAGGGCAAA | 41 | CGGATTTTATCAACG | 91 |
| | | TGGAAGG | | ATGCAG | |
| PGR | NM_000926 | TGCCGCAGAACTCA | 42 | CATTTGCCGTCCTTC | 92 |
| | | CTTG | | ATCG | |
| PHGDH | AK093306 | CCTCAGATGATGCC | 43 | GCAGGTCAAAACTC | 93 |
| | | TATCCA | | TCAAAG | |
| PTTG1 | BE904476 | CAGCAAGCGATGGC | 44 | AGCGGGCTTCTGTAA | 94 |
| | | ATAGT | | TCTGA | |
| RRM2 | AK123010 | AATGCCACCGAAGC | 45 | GCCTCAGATTTCAAC | 95 |
| | | CTC | | TCGT | |
| SFRP1 | BC036503 | TCGAACTGAAGGCT | 46 | CTGCTGAGAATCAA | 96 |
| | | ATTTACGAG | | AGTGGGA | |
| SLC39A6 | NM_012319 | GTCGAAGCCGCAAT | 47 | GGAACAAACTGCTC | 97 |
| | | TAGG | | TGCCA | |
| TMEM45B | AK098106 | CAAACGTGTGTTCT | 48 | ACAGCTCTTTAGCAT | 98 |
| | | GGAGG | | TTGTGGA | |
| TYMS | BQ56428 | TGCCCTGTATGATGT | 49 | GGGACTATCAATGTT | 99 |
| | | CAGGA | | GGGTTCTC | |
| UBE2C | BC032677 | GTGAGGGGTGTCAG | 50 | CACACAGTTCACTGC | 100 |
| | | CTCAGT | | TCCACA | |

TABLE 2
Exemplary Probes for detecting NANO46 genes
Table 2

| | | | SEQ |
|---|---|---|---|
| | | | ID |
| Gene Name | Ref Seq Accession | Target Sequence | NO: |
| ACTR3B | NM_001040135.1 | CCAGAAGAAGTTTGTTATAGACGTTGGTTACGAA | 101 |
| | | AGATTCCTGGGACCTGAAATATTCTTTCACCCGGA | |
| | | GTTTGCCAACCCAGACTTTATGGAGTCCATC | |
| ANLN | NM_018685.2 | CGTGCCAGGCGAGAGAATCTTCAGAGAAAAATGG | 102 |
| | | CTGAGAGGCCCACAGCAGCTCCAAGGTCTATGAC | |
| | | TCATGCTAAGCGAGCTAGACAGCCACTTTCAG | |
| BAG1 | NM_004323.3 | CTTCATGTTACCTCCCAGCAGGGCAGCAGTGAAC | 103 |
| | | CAGTTGTCCAAGACCTGGCCCAGGTTGTTGAAGA | |
| | | GGTCATAGGGGTTCCACAGTCTTTTCAGAAAC | |
| BCL2 | NM_000633.2 | CCAAGCACCGCTTCGTGTGGCTCCACCTGGATGTT | 104 |
| | | CTGTGCCTGTAAACATAGATTCGCTTTCCATGTTG | |
| | | TTGGCCGGATCACCATCTGAAGAGCAGACG | |
| BLVRA | NM_000712.3 | TTCCTGAAAAAAGAAGTGGTGGGGAAAGACCTGC | 105 |
| | | TGAAAGGGTCGCTCCTCTTCACAGCTGGCCCGTTG | |
| | | GAAGAAGAGCGGTTTGGCTTCCCTGCATTCA | |
| CCNE1 | NM_001238.1 | GAGAACTGTGTCAAGTGGATGGTTCCATTTGCCA | 106 |
| | | TGGTTATAAGGGAGACGGGGAGCTCAAAACTGAA | |
| | | GCACTTCAGGGGCGTCGCTGATGAAGATGCAC | |
| CDC20 | NM_001255.1 | CCCGAGTGGGCTCCCTAAGCTGGAACAGCTATAT | 107 |
| | | CCTGTCCAGTGGTTCACGTTCTGGCCACATCCACC | |
| | | ACCATGATGTTCGGGTAGCAGAACACCATGT | |
| CDC6 | NM_001254.3 | GGGGAAGTTATATGAAGCCTACAGTAAAGTCTGT | 108 |
| | | CGCAAACAGCAGGTGGCGGCTGTGGACCAGTCAG | |
| | | AGTGTTTGTCACTTTCAGGGCTCTTGGAAGCC | |
| CDCA1 | NM_145697.1 | GCCTGGCGGTGTTTTCGTCGTGCTCAGCGGTGGG | 109 |
| | | AGGAGGCGGAAGAAACCAGAGCCTGGGAGATTA | |
| | | ACAGGAAACTTCCAAGATGGAAACTTTGTCTTT | |
| CDH3 | NM_001793.3 | CCCTCGACCGTGAGGATGAGCAGTTTGTGAGGAA | 110 |
| | | CAACATCTATGAAGTCATGGTCTTGGCCATGGAC | |
| | | AATGGAAGCCCTCCCACCACTGGCACGGGAAC | |
| CENPF | NM_016343.3 | AGAAAATCTTGCAGAGTCCTCCAAACCAACAGCT | 111 |
| | | GGTGGCAGCAGATCACAAAAGGTCAAAGTTGCTC | |
| | | AGCGGAGCCCAGTAGATTCAGGCACCATCCTC | |
| CEP55 | NM_018131.3 | GTACTACCGCATTGCTTGAACAGCTGGAAGAGAC | 112 |
| | | AACGAGAGAAGGAGAAAGGAGGGAGCAGGTGTT | |
| | | GAAAGCCTTATCTGAAGAGAAAGACGTATTGAA | |
| CXXC5 | NM_016463.5 | AGCTGCCCTCTCCGTGCAATGTCACTGCTCGTGTG | 113 |
| | | GTCTCCAGCAAGGGATTCGGGCGAAGACAAACGG | |
| | | ATGCACCCGTCTTTAGAACCAAAAATATTCT | |
| EGFR | NM_005228.3 | GCAGCCAGGAACGTACTGGTGAAAACACCGCAGC | 114 |
| | | ATGTCAAGATCACAGATTTTGGGCTGGCCAAACT | |
| | | GCTGGGTGCGGAAGAGAAAGAATACCATGCAG | |
| ERBB2 | NM_004448.2 | TGAAGGTGCTTGGATCTGGCGCTTTTGGCACAGTC | 115 |
| | | TACAAGGGCATCTGGATCCCTGATGGGGAGAATG | |
| | | TGAAAATTCCAGTGGCCATCAAAGTGTTGAG | |
| ESR1 | NM_000125.2 | AGGAACCAGGGAAAATGTGTAGAGGGCATGGTG | 116 |
| | | GAGATCTTCGACATGCTGCTGGCTACATCATCTCG | |
| | | GTTCCGCATGATGAATCTGCAGGGAGAGGAGT | |
| EXO1 | NM_006027.3 | TGGCCCACAAAGTAATTAAAGCTGCCCGGTCTCA | 117 |
| | | GGGGGTAGATTGCCTCGTGGCTCCCTATGAAGCT | |
| | | GATGCGCAGTTGGCCTATCTTAACAAAGCGGG | |
| FGFR4 | NM_002011.3 | CCCACATCCAGTGGCTGAAGCACATCGTCATCAA | 118 |
| | | CGGCAGCAGCTTCGGAGCCGACGGTTTCCCCTAT | |
| | | GTGCAAGTCCTAAAGACTGCAGACATCAATAG | |
| FOXA1 | NM_004496.2 | TGGATGGTTGTATTGGGCAGGGTGGCTCCAGGAT | 119 |
| | | GTTAGGAACTGTGAAGATGGAAGGGCATGAAACC | |
| | | AGCGACTGGAACAGCTACTACGCAGACACGCA | |
| FOXC1 | NM_001453.1 | TTCGAGTCACAGAGGATCGGCTTGAACAACTCTC | 120 |
| | | CAGTGAACGGGAATAGTAGCTGTCAAATGGCCTT | |
| | | CCCTTCCAGCCAGTCTCTGTACCGCACGTCCG | |
| GPR160 | NM_014373.1 | GGATTTCAGTCCTTGCTTATGTTTTGGGAGACCCA | 121 |
| | | GCCATCTACCAAAGCCTGAAGGCACAGAATGCTT | |
| | | ATTCTCGTCACTGTCCTTTCTATGTCAGCAT | |
| UBE2T | NM_014176.1 | GTGTCAGCTCAGTGCATCCCAGGCAGCTCTTAGT | 122 |
| | | GTGGAGCAGTGAACTGTGTGTGGTTCCTTCTACTT | |
| | | GGGGATCATGCAGAGAGCTTCACGTCTGAAG | |
| KIF2C | NM_006845.2 | GTTGTCTACAGGTTCACAGCAAGGCCACTGGTAC | 123 |
| | | AGACAATCTTTGAAGGTGGAAAAGCAACTTGTTT | |
| | | TGCATATGGCCAGACAGGAAGTGGCAAGACAC | |
| KNTC2 | NM_006101.1 | AAAAGGTCATAAGCATGAAGCGCAGTTCAGTTTC | 124 |
| | | CAGCGGTGGTGCTGGCCGCCTCTCCATGCAGGAG | |
| | | TTAAGATCCCAGGATGTAAATAAACAAGGCCT | |
| KRT14 | NM_000526.3 | GCAGTCATCCAGAGATGTGACCTCCTCCAGCCGC | 125 |
| | | CAAATCCGCACCAAGGTCATGGATGTGCACGATG | |
| | | GCAAGGTGGTGTCCACCCACGAGCAGGTCCTT | |
| KRT17 | NM_000422.1 | CTGACTCAGTACAAGAAAGAACCGGTGACCACCC | 126 |
| | | GTCAGGTGCGTACCATTGTGGAAGAGGTCCAGGA | |
| | | TGGCAAGGTCATCTCCTCCCGCGAGCAGGTCC | |
| KRT5 | NM_000424.2 | CTGGTTCTCTTGCTCCACCAGGAACAAGCCACCAT | 127 |
| | | GTCTCGCCAGTCAAGTGTGTCCTTCCGGAGCGGG | |
| | | GGCAGTCGTAGCTTCAGCACCGCCTCTGCCA | |
| MAPT | NM_016835.3 | GCCGGGTCCCTCAACTCAAAGCTCGCATGGTCAG | 128 |
| | | TAAAAGCAAAGACGGGACTGGAAGCGATGACAA | |
| | | AAAAGCCAAGACATCCACACGTTCCTCTGCTAA | |
| MDM2 | NM_006878.2 | GGTGAGGAGCAGGCAAATGTGCAATACCAACATG | 129 |
| | | TCTGTACCTACTGATGGTGCTGTAACCACCTCACA | |
| | | GATTCCAGCTTCGGAACAAGAGACCCTGGTT | |
| MELK | NM_014791.2 | AGAGACAGCCAACAAAATATTCATGGTTCTTGAG | 130 |
| | | TACTGCCCTGGAGGAGAGCTGTTTGACTATATAA | |
| | | TTTCCCAGGATCGCCTGTCAGAAGAGGAGACC | |
| MIA | NM_006533.1 | CCGGGGCCAAGTGGTGTATGTCTTCTCCAAGCTG | 131 |
| | | AAGGGCCGTGGGCGGCTCTTCTGGGGAGGCAGCG | |
| | | TTCAGGGAGATTACTATGGAGATCTGGCTGCT | |
| MKI67 | NM_002417.2 | GCTTCCAGCAGCAAATCTCAGACAGAGGTTCCTA | 132 |
| | | AGAGAGGAGGAGAAAGAGTGGCAACCTGCCTTC | |
| | | AAAAGAGAGTGTCTATCAGCCGAAGTCAACATG | |
| MLPH | NM_024101.4 | GAGGAAGTCAAACCTCCCGATATTTCTCCCTCGA | 133 |
| | | GTGGCTGGGAAACTTGGCAAGAGACCAGAGGAC | |
| | | CCAAATGCAGACCCTTCAAGTGAGGCCAAGGCA | |
| MMP11 | NM_005940.3 | AGCAGCCAAGGCCCTGATGTCCGCCTTCTACACC | 134 |
| | | TTTCGCTACCCACTGAGTCTCAGCCCAGATGACTG | |
| | | CAGGGGCGTTCAACACCTATATGGCCAGCCC | |
| MYC | NM_002467.3 | CACCGAGGAGAATGTCAAGAGGCGAACACACAA | 135 |
| | | CGTCTTGGAGCGCCAGAGGAGGAACGAGCTAAA | |
| | | ACGGAGCTTTTTTGCCCTGCGTGACCAGATCCCG | |
| NAT1 | NM_000662.4 | AGCACTTCCTCATAGACCTTGGATGTGGGAGGAT | 136 |
| | | TGCATTCAGTCTAGTTCCTGGTTGCCGGCTGAAAT | |
| | | AACCTGAATTCAAGCCAGGAAGAAGCAGCAA | |
| ORC6L | NM_014321.2 | GACTGTGTAAACAACTAGAGAAGATTGGACAGCA | 137 |
| | | GGTCGACAGAGAACCTGGAGATGTAGCTACTCCA | |
| | | CCACGGAAGAGAAAGAAGATAGTGGTTGAAGC | |
| PGR | NM_000926.2 | GGGATGAAGCATCAGGCTGTCATTATGGTGTCCT | 138 |
| | | TACCTGTGGGAGCTGTAAGGTCTTCTTTAAGAGG | |
| | | GCAATGGAAGGGCAGCACAACTACTTATGTGC | |
| PHGDH | NM_006623.2 | GCGACGGCTTCGATGAAGGACGGCAAATGGGAG | 139 |
| | | CGGAAGAAGTTCATGGGAACAGAGCTGAATGGA | |
| | | AAGACCCTGGGAATTCTTGGCCTGGGCAGGATTG | |
| PTTG1 | NM_004219.2 | CACCAGCCTTACCTAAAGCTACTAGAAAGGCTTT | 140 |
| | | GGGAACTGTCAACAGAGCTACAGAAAAGTCTGTA | |
| | | AAGACCAAGGGACCCCTCAAACAAAAACAGCC | |
| RRM2 | NM_001034.1 | TTCCTTTTGGACCGCCGAGGAGGTTGACCTCTCCA | 141 |
| | | AGGACATTCAGCACTGGGAATCCCTGAAACCCGA | |
| | | GGAGAGATATTTTATATCCCATGTTCTGGCT | |
| SFRP1 | NM_003012.3 | GTGGGTCACACACACGCACTGCGCCTGTCAGTAG | 142 |
| | | TGGACATTGTAATCCAGTCGGCTTGTTCTTGCAGC | |
| | | ATTCCCGCTCCCTTCCCTCCATAGCCACGCT | |
| SLC39A6 | NM_012319.2 | GATCGAACTGAAGGCTATTTACGAGCAGACTCAC | 143 |
| | | AAGAGCCCTCCCACTTTGATTCTCAGCAGCCTGCA | |
| | | GTCTTGGAAGAAGAAGAGGTCATGATAGCTC | |
| TMEM45B | NM_138788.3 | CTGGCTGCCCTCAGCATTGTGGCCGTCAACTATTC | 144 |
| | | TCTTGTTTACTGCCTTTTGACTCGGATGAAGAGAC | |
| | | ACGGAAGGGGAGAAATCATTGGAATTCAGA | |
| TYMS | NM_001071.1 | TGCTAAAGAGCTGTCTTCCAAGGGAGTGAAAATC | 145 |
| | | TGGGATGCCAATGGATCCCGAGACTTTTTGGACA | |
| | | GCCTGGGATTCTCCACCAGAGAAGAAGGGGAC | |
| UBE2C | NM_007019.2 | GTCTGCCCTGTATGATGTCAGGACCATTCTGCTCT | 146 |
| | | CCATCCAGAGCCTTCTAGGAGAACCCAACATTGA | |
| | | TAGTCCCTTGAACACACATGCTGCCGAGCTC | |

Table 3 provides select sequences for the PAM50 genes of Table 1.

TABLE 3

| GENBANK | | |
|---|---|---|
| ACCESSION | | SEQ |
| NUMBER | SEQUENCE | ID NO: |
| NM_020445 | CAGCGGCGCTGCGGCGGCTCGCGGGAGACGCTGCGCGCGGGGCTAGCG | 147 |
| | GGCGGCGGAGCGGACGGCGACGGGGCGCTCTCGGGCTGCCGGCGGGGC | |
| | CGAGCGCCGCGCGTCCCGAGCATGGCAGGCTCCCTGCCTCCCTGCGTGG | |
| | TGGACTGTGGCACCGGGTATACCAAGCTTGGCTACGCAGGCAACACTG | |
| | AGCCCCAGTTCATTATTCCTTCATGTATTGCCATCAGAGAGTCAGCAAA | |
| | GGTAGTTGACCAAGCTCAAAGGAGAGTGTTGAGGGGAGTTGATGACCT | |
| | TGACTTTTTCATAGGAGATGAAGCCATCGATAAACCTACATATGCTACA | |
| | AAGTGGCCGATACGACATGGAATCATTGAAGACTGGGATCTTATGGAA | |
| | AGGTTCATGGAGCAAGTGGTTTTTAAATATCTTCGAGCTGAACCTGAGG | |
| | ACCATTATTTTTTAATGACAGAACCTCCACTCAATACACCAGAAAACAG | |
| | AGAGTATCTTGCAGAAATTATGTTTGAATCATTTAACGTACCAGGACTC | |
| | TACATTGCAGTTCAGGCAGTGCTGGCCTTGGCGGCATCTTGGACATCTC | |
| | GACAAGTGGGTGAACGTACGTTAACGGGGATAGTCATTGACAGCGGAG | |
| | ATGGAGTCACCCATGTTATCCCAGTGGCAGAAGGTTATGTAATTGGAAG | |
| | CTGCATCAAACACATCCCGATTGCAGGTAGAGATATTACGTATTTCATT | |
| | CAACAGCTGCTAAGGGAGAGGGAGGTGGGAATCCCTCCTGAGCAGTCA | |
| | CTGGAGACCGCAAAAGCCATTAAGGAGAAATACTGTTACATTTGCCCC | |
| | GATATAGTCAAGGAATTTGCCAAGTATGATGTGGATCCCCGGAAGTGG | |
| | ATCAAACAGTACACGGGTATCAATGCGATCAACCAGAAGAAGTTTGTT | |
| | ATAGACGTTGGTTACGAAAGATTCCTGGGACCTGAAATATTCTTTCACC | |
| | CGGAGTTTGCCAACCCAGACTTTATGGAGTCCATCTCAGATGTTGTTGA | |
| | TGAAGTAATACAGAACTGCCCCATCGATGTGCGGCGCCCGCTGTATAAG | |
| | AATGTCGTACTCTCAGGAGGCTCCACCATGTTCAGGGATTTCGGACGCC | |
| | GACTGCAGAGGGATTTGAAGAGAGTGGTGGATGCTAGGCTGAGGCTCA | |
| | GCGAGGAGCTCAGCGGCGGGAGGATCAAGCCGAAGCCTGTGGAGGTCC | |
| | AGGTGGTCACGCATCACATGCAGCGCTACGCCGTGTGGTTCGGAGGCTC | |
| | CATGCTGGCCTCGACTCCCGAGTTCTTTCAGGTCTGCCACACCAAGAAG | |
| | GACTATGAAGAGTACGGGCCCAGCATCTGCCGCCACAACCCCGTCTTTG | |
| | GAGTCATGTCCTAGTGTCTGCCTGAACGCGTCGTTCGATGGTGTCACGT | |
| | TGGGGAACAAGTGTCCTTCAGAACCCAGAGAAGGCCGCCGTTCTGTAA | |
| | ATAGCGACGTCGGTGTTGCTGCCCAGCAGCGTGCTTGCATTGCCGGTGC | |
| | ATGAGGCGCGGCGCGGGCCCTTCAGTAAAAGCCATTTATCCGTGTGCCG | |
| | ACCGCTGTCTGCCAGCCTCCTCCTTCTCCCGCCCTCCTCACCCTCGCTCT | |
| | CCCTCCTCCTCCTCCTCCGAGCTGCTAGCTGACAAATACAATTCTGAAG | |
| | GAATCCAAATGTGACTTTGAAAATTGTTAGAGAAAACAACATTAGAAA | |
| | ATGGCGCAAAATCGTTAGGTCCCAGGAGAGAATGTGGGGGCGCAAACC | |
| | CTTTTCCTCCCAGCCTATTTTTGTAAATAAAATGTTTAAACTTGAAATAC | |
| | AAATCGATGTTTATATTTCCTATCATTTTGTATTTTATGGTATTTGGTAC | |
| | AACTGGCTGATACTAAGCACGAATAGATATTGATGTTATGGAGTGCTGT | |
| | AATCCAAAGTTTTTAATTGTGAGGCATGTTCTGATATGTTTATAGGCAA | |
| | ACAAATAAAACAGCAAACTTTTTTGCCACATGTTTGCTAGAAAATGATT | |
| | ATACTTTATTGGAGTGACATGAAGTTTGAACACTAAACAGTAATGTATG | |
| | AGAATTACTACAGATACATGTATCTTTTAGTTTTTTTTGTTTGAACTTTC | |
| | TGGAGCTGTTTTATAGAAGATGATGGTTTGTTGTCGGTGAGTGTTGGAT | |
| | GAAATACTTCCTTGCACCATTGTAATAAAAGCTGTTAGAATATTTGTAA | |
| | ATATC | |
| NM_001040135 | CAGCGGCGCTGCGGCGGCTCGCGGGAGACGCTGCGCGCGGGGCTAGCG | 148 |
| | GGCGGCGGAGCGGACGGCGACGGGGCGCTCTCGGGCTGCCGGCGGGGC | |
| | CGAGCGCCGCGCGTCCCGAGCATGGCAGGCTCCCTGCCTCCCTGCGTGG | |
| | TGGACTGTGGCACCGGGTATACCAAGCTTGGCTACGCAGGCAACACTG | |
| | AGCCCCAGTTCATTATTCCTTCATGTATTGCCATCAGAGAGTCAGCAAA | |
| | GGTAGTTGACCAAGCTCAAAGGAGAGTGTTGAGGGGAGTTGATGACCT | |
| | TGACTTTTTCATAGGAGATGAAGCCATCGATAAACCTACATATGCTACA | |
| | AAGTGGCCGATACGACATGGAATCATTGAAGACTGGGATCTTATGGAA | |
| | AGGTTCATGGAGCAAGTGGTTTTTAAATATCTTCGAGCTGAACCTGAGG | |
| | ACCATTATTTTTTAATGACAGAACCTCCACTCAATACACCAGAAAACAG | |
| | AGAGTATCTTGCAGAAATTATGTTTGAATCATTTAACGTACCAGGACTC | |
| | TACATTGCAGTTCAGGCAGTGCTGGCCTTGGCGGCATCTTGGACATCTC | |
| | GACAAGTGGGTGAACGTACGTTAACGGGGATAGTCATTGACAGCGGAG | |
| | ATGGAGTCACCCATGTTATCCCAGTGGCAGAAGGTTATGTAATTGGAAG | |
| | CTGCATCAAACACATCCCGATTGCAGGTAGAGATATTACGTATTTCATT | |
| | CAACAGCTGCTAAGGGAGAGGGAGGTGGGAATCCCTCCTGAGCAGTCA | |
| | CTGGAGACCGCAAAAGCCATTAAGGAGAAATACTGTTACATTTGCCCC | |
| | GATATAGTCAAGGAATTTGCCAAGTATGATGTGGATCCCCGGAAGTGG | |
| | ATCAAACAGTACACGGGTATCAATGCGATCAACCAGAAGAAGTTTGTT | |
| | ATAGACGTTGGTTACGAAAGATTCCTGGGACCTGAAATATTCTTTCACC | |
| | CGGAGTTTGCCAACCCAGACTTTATGGAGTCCATCTCAGATGTTGTTGA | |
| | TGAAGTAATACAGAACTGCCCCATCGATGTGCGGCGCCCGCTGTATAAG | |
| | CCCGAGTTCTTTCAGGTCTGCCACACCAAGAAGGACTATGAAGAGTACG | |
| | GGCCCAGCATCTGCCGCCACAACCCCGTCTTTGGAGTCATGTCCTAGTG | |
| | TCTGCCTGAACGCGTCGTTCGATGGTGTCACGTTGGGGAACAAGTGTCC | |
| | TTCAGAACCCAGAGAAGGCCGCCGTTCTGTAAATAGCGACGTCGGTGTT | |
| | GCTGCCCAGCAGCGTGCTTGCATTGCCGGTGCATGAGGCGCGGCGCGG | |
| | GCCCTTCAGTAAAAGCCATTTATCCGTGTGCCGACCGCTGTCTGCCAGC | |
| | CTCCTCCTTCTCCCGCCCTCCTCACCCTCGCTCTCCCTCCTCCTCCTCCTC | |
| | CGAGCTGCTAGCTGACAAATACAATTCTGAAGGAATCCAAATGTGACTT | |
| | TGAAAATTGTTAGAGAAAACAACATTAGAAAATGGCGCAAAATCGTTA | |
| | GGTCCCAGGAGAGAATGTGGGGGCGCAAACCCTTTTCCTCCCAGCCTAT | |
| | TTTTGTAAATAAAATGTTTAAACTTGAAATACAAATCGATGTTTATATTT | |
| | CCTATCATTTTGTATTTTATGGTATTTGGTACAACTGGCTGATACTAAGC | |
| | ACGAATAGATATTGATGTTATGGAGTGCTGTAATCCAAAGTTTTTAATT | |
| | GTGAGGCATGTTCTGATATGTTTATAGGCAAACAAATAAAACAGCAAA | |
| | CTTTTTTGCCACATGTTTGCTAGAAAATGATTATACTTTATTGGAGTGAC | |
| | ATGAAGTTTGAACACTAAACAGTAATGTATGAGAATTACTACAGATAC | |
| | ATGTATCTTTTAGTTTTTTTTGTTTGAACTTTCTGGAGCTGTTTTATAGAA | |
| | GATGATGGTTTGTTGTCGGTGAGTGTTGGATGAAATACTTCCTTGCACC | |
| | ATTGTAATAAAAGCTGTTAGAATATTTGTAAATATC | |
| NM_018685 | CTCGGCGCTGAAATTCAAATTTGAACGGCTGCAGAGGCCGAGTCCGTCA | 149 |
| | CTGGAAGCCGAGAGGAGAGGACAGCTGGTTGTGGGAGAGTTCCCCCGC | |
| | CTCAGACTCCTGGTTTTTTCCAGGAGACACACTGAGCTGAGACTCACTT | |
| | TTCTCTTCCTGAATTTGAACCACCGTTTCCATCGTCTCGTAGTCCGACGC | |
| | CTGGGGCGATGGATCCGTTTACGGAGAAACTGCTGGAGCGAACCCGTG | |
| | CCAGGCGAGAGAATCTTCAGAGAAAAATGGCTGAGAGGCCCACAGCAG | |
| | CTCCAAGGTCTATGACTCATGCTAAGCGAGCTAGACAGCCACTTTCAGA | |
| | AGCAAGTAACCAGCAGCCCCTCTCTGGTGGTGAAGAGAAATCTTGTAC | |
| | AAAACCATCGCCATCAAAAAAACGCTGTTCTGACAACACTGAAGTAGA | |
| | AGTTTCTAACTTGGAAAATAAACAACCAGTTGAGTCGACATCTGCAAAA | |
| | TCTTGTTCTCCAAGTCCTGTGTCTCCTCAGGTGCAGCCACAAGCAGCAG | |
| | ATACCATCAGTGATTCTGTTGCTGTCCCGGCATCACTGCTGGGCATGAG | |
| | GAGAGGGCTGAACTCAAGATTGGAAGCAACTGCAGCCTCCTCAGTTAA | |
| | AACACGTATGCAAAAACTTGCAGAGCAACGGCGCCGTTGGGATAATGA | |
| | TGATATGACAGATGACATTCCTGAAAGCTCACTCTTCTCACCAATGCCA | |
| | TCAGAGGAAAAGGCTGCTTCCCCTCCCAGACCTCTGCTTTCAAATGCCT | |
| | CGGCAACTCCAGTTGGCAGAAGGGGCCGTCTGGCCAATCTTGCTGCAAC | |
| | TATTTGCTCCTGGGAAGATGATGTAAATCACTCATTTGCAAAACAAAAC | |
| | AGTGTACAAGAACAGCCTGGTACCGCTTGTTTATCCAAATTTTCCTCTG | |
| | CAAGTGGAGCATCTGCTAGGATCAATAGCAGCAGTGTTAAGCAGGAAG | |
| | CTACATTCTGTTCCCAAAGGGATGGCGATGCCTCTTTGAATAAAGCCCT | |
| | ATCCTCAAGTGCTGATGATGCGTCTTTGGTTAATGCCTCAATTTCCAGCT | |
| | CTGTGAAAGCTACTTCTCCAGTGAAATCTACTACATCTATCACTGATGC | |
| | TAAAAGTTGTGAGGGACAAAATCCTGAGCTACTTCCAAAAACTCCTATT | |
| | AGTCCTCTGAAAACGGGGGTATCGAAACCAATTGTGAAGTCAACTTTAT | |
| | CCCAGACAGTTCCATCCAAGGGAGAATTAAGTAGAGAAATTTGTCTGC | |
| | AATCTCAATCTAAAGACAAATCTACGACACCAGGAGGAACAGGAATTA | |
| | AGCCTTTCCTGGAACGCTTTGGAGAGCGTTGTCAAGAACATAGCAAAG | |
| | AAAGTCCAGCTCGTAGCACACCCCACAGAACCCCCATTATTACTCCAAA | |
| | TACAAAGGCCATCCAAGAAAGATTATTCAAGCAAGACACATCTTCATCT | |
| | ACTACCCATTTAGCACAACAGCTCAAGCAGGAACGTCAAAAAGAACTA | |
| | GCATGTCTTCGTGGCCGATTTGACAAGGGCAATATATGGAGTGCAGAA | |
| | AAAGGCGGAAACTCAAAAAGCAAACAACTAGAAACCAAACAGGAAAC | |
| | TCACTGTCAGAGCACTCCCCTCAAAAAACACCAAGGTGTTTCAAAAACT | |
| | CAGTCACTTCCAGTAACAGAAAAGGTGACCGAAAACCAGATACCAGCC | |
| | AAAAATTCTAGTACAGAACCTAAAGGTTTCACTGAATGCGAAATGACG | |
| | AAATCTAGCCCTTTGAAAATAACATTGTTTTTAGAAGAGGACAAATCCT | |
| | TAAAAGTAACATCAGACCCAAAGGTTGAGCAGAAAATTGAAGTGATAC | |
| | GTGAAATTGAGATGAGTGTGGATGATGATGATATCAATAGTTCGAAAG | |
| | TAATTAATGACCTCTTCAGTGATGTCCTAGAGGAAGGTGAACTAGATAT | |
| | GGAGAAGAGCCAAGAGGAGATGGATCAAGCATTAGCAGAAAGCAGCG | |
| | AAGAACAGGAAGATGCACTGAATATCTCCTCAATGTCTTTACTTGCACC | |
| | ATTGGCACAAACAGTTGGTGTGGTAAGTCCAGAGAGTTTAGTGTCCACA | |
| | CCTAGACTGGAATTGAAAGACACCAGCAGAAGTGATGAAAGTCCAAAA | |
| | CCAGGAAAATTCCAAAGAACTCGTGTCCCTCGAGCTGAATCTGGTGATA | |
| | GCCTTGGTTCTGAAGATCGTGATCTTCTTTACAGCATTGATGCATATAG | |
| | ATCTCAAAGATTCAAAGAAACAGAACGTCCATCAATAAAGCAGGTGAT | |
| | TGTTCGGAAGGAAGATGTTACTTCAAAACTGGATGAAAAAAATAATGC | |
| | CTTTCCTTGTCAAGTTAATATCAAACAGAAAATGCAGGAACTCAATAAC | |
| | GAAATAAATATGCAACAGACAGTGATCTATCAAGCTAGCCAGGCTCTT | |
| | AACTGCTGTGTTGATGAAGAACATGGAAAAGGGTCCCTAGAAGAAGCT | |
| | GAAGCAGAAAGACTTCTTCTAATTGCAACTGGGAAGAGAACACTTTTG | |
| | ATTGATGAATTGAATAAATTGAAGAACGAAGGACCTCAGAGGAAGAAT | |
| | AAGGCTAGTCCCCAAAGTGAATTTATGCCATCCAAAGGATCAGTTACTT | |
| | TGTCAGAAATCCGCTTGCCTCTAAAAGCAGATTTTGTCTGCAGTACGGT | |
| | TCAGAAACCAGATGCAGCAAATTACTATTACTTAATTATACTAAAAGCA | |
| | GGAGCTGAAAATATGGTAGCCACACCATTAGCAAGTACTTCAAACTCTC | |
| | TTAACGGTGATGCTCTGACATTCACTACTACATTTACTCTGCAAGATGT | |
| | ATCCAATGACTTTGAAATAAATATTGAAGTTTACAGCTTGGTGCAAAAG | |
| | AAAGATCCCTCAGGCCTTGATAAGAAGAAAAAAACATCCAAGTCCAAG | |
| | GCTATTACTCCAAAGCGACTCCTCACATCTATAACCACAAAAAGCAACA | |
| | TTCATTCTTCAGTCATGGCCAGTCCAGGAGGTCTTAGTGCTGTGCGAAC | |
| | CAGCAACTTCGCCCTTGTTGGATCTTACACATTATCATTGTCTTCAGTAG | |
| | GAAATACTAAGTTTGTTCTGGACAAGGTCCCCTTTTTATCTTCTTTGGAA | |
| | GGTCATATTTATTTAAAAATAAAATGTCAAGTGAATTCCAGTGTTGAAG | |
| | AAAGAGGTTTTCTAACCATATTTGAAGATGTTAGTGGTTTTGGTGCCTG | |
| | GCATCGAAGATGGTGTGTTCTTTCTGGAAACTGTATATCTTATTGGACTT | |
| | ATCCAGATGATGAGAAACGCAAGAATCCCATAGGAAGGATAAATCTGG | |
| | CTAATTGTACCAGTCGTCAGATAGAACCAGCCAACAGAGAATTTTGTGC | |
| | AAGACGCAACACTTTTGAATTAATTACTGTCCGACCACAAAGAGAAGA | |
| | TGACCGAGAGACTCTTGTCAGCCAATGCAGGGACACACTCTGTGTTACC | |
| | AAGAACTGGCTGTCTGCAGATACTAAAGAAGAGCGGGATCTCTGGATG | |
| | CAAAAACTCAATCAAGTTCTTGTTGATATTCGCCTCTGGCAACCTGATG | |
| | CTTGCTACAAACCTATTGGAAAGCCTTAAACCGGGAAATTTCCATGCTA | |
| | TCTAGAGGTTTTTGATGTCATCTTAAGAAACACACTTAAGAGCATCAGA | |
| | TTTACTGATTGCATTTTATGCTTTAAGTACGAAAGGGTTTGTGCCAATAT | |
| | TCACTACGTATTATGCAGTATTTATATCTTTTGTATGTAAAACTTTAACT | |
| | GATTTCTGTCATTCATCAATGAGTAGAAGTAAATACATTATAGTTGATT | |
| | TTGCTAAATCTTAATTTAAAAGCCTCATTTTCCTAGAAATCTAATTATTC | |
| | AGTTATTCATGACAATATTTTTTTAAAAGTAAGAAATTCTGAGTTGTCTT | |
| | CTTGGAGCTGTAGGTCTTGAAGCAGCAACGTCTTTCAGGGGTTGGAGAC | |
| | AGAAACCCATTCTCCAATCTCAGTAGTTTTTTCGAAAGGCTGTGATCAT | |
| | TTATTGATCGTGATATGACTTGTTACTAGGGTACTGAAAAAAATGTCTA | |
| | AGGCCTTTACAGAAACATTTTTAGTAATGAGGATGAGAACTTTTTCAAA | |
| | TAGCAAATATATATTGGCTTAAAGCATGAGGCTGTCTTCAGAAAAGTGA | |
| | TGTGGACATAGGAGGCAATGTGTGAGACTTGGGGGTTCAATATTTTATA | |
| | TAGAAGAGTTAATAAGCACATGGTTTACATTTACTCAGCTACTATATAT | |
| | GCAGTGTGGTGCACATTTTCACAGAATTCTGGCTTCATTAAGATCATTA | |
| | TTTTTGCTGCGTAGCTTACAGACTTAGCATATTAGTTTTTTCTACTCCTA | |
| | CAAGTGTAAATTGAAAAATCTTTATATTAAAAAAGTAAACTGTTATGAA | |
| | GCTGCTATGTACTAATAATACTTTGCTTGCCAAAGTGTTTGGGTTTTGTT | |
| | GTTGTTTGTTTGTTTGTTTGTTTTTGGTTCATGAACAACAGTGTCTAGAA | |
| | ACCCATTTTGAAAGTGGAAAATTATTAAGTCACCTATCACCTTTAAACG | |
| | CCTTTTTTTAAAATTATAAAATATTGTAAAGCAGGGTCTCAACTTTTAAA | |
| | TACACTTTGAACTTCTTCTCTGAATTATTAAAGTTCTTTATGACCTCATT | |
| | TATAAACACTAAATTCTGTCACCTCCTGTCATTTTATTTTTTATTCATTCA | |
| | AATGTATTTTTTCTTGTGCATATTATAAAAATATATTTTATGAGCTCTTA | |
| | CTCAAATAAATACCTGTAAATGTCTAAAGGAAAAAAAAAAAAAAAAAA | |
| NM_004323 | AGGCCGGGGCGGGGCTGGGAAGTAGTCGGGCGGGGTTGTGAGACGCCG | 150 |
| | CGCTCAGCTTCCATCGCTGGGCGGTCAACAAGTGCGGGCCTGGCTCAGC | |
| | GCGGGGGGGCGCGGAGACCGCGAGGCGACCGGGAGCGGCTGGGTTCCC | |
| | GGCTGCGCGCCCTTCGGCCAGGCCGGGAGCCGCGCCAGTCGGAGCCCC | |
| | CGGCCCAGCGTGGTCCGCCTCCCTCTCGGCGTCCACCTGCCCGGAGTAC | |
| | TGCCAGCGGGCATGACCGACCCACCAGGGGCGCCGCCGCCGGCGCTCG | |
| | CAGGCCGCGGATGAAGAAGAAAACCCGGCGCCGCTCGACCCGGAGCGA | |
| | GGAGTTGACCCGGAGCGAGGAGTTGACCCTGAGTGAGGAAGCGACCTG | |
| | GAGTGAAGAGGCGACCCAGAGTGAGGAGGCGACCCAGGGCGAAGAGA | |
| | TGAATCGGAGCCAGGAGGTGACCCGGGACGAGGAGTCGACCCGGAGCG | |
| | AGGAGGTGACCAGGGAGGAAATGGCGGCAGCTGGGCTCACCGTGACTG | |
| | TCACCCACAGCAATGAGAAGCACGACCTTCATGTTACCTCCCAGCAGGG | |
| | CAGCAGTGAACCAGTTGTCCAAGACCTGGCCCAGGTTGTTGAAGAGGT | |
| | CATAGGGGTTCCACAGTCTTTTCAGAAACTCATATTTAAGGGAAAATCT | |
| | CTGAAGGAAATGGAAACACCGTTGTCAGCACTTGGAATACAAGATGGT | |
| | TGCCGGGTCATGTTAATTGGGAAAAAGAACAGTCCACAGGAAGAGGTT | |
| | GAACTAAAGAAGTTGAAACATTTGGAGAAGTCTGTGGAGAAGATAGCT | |
| | GACCAGCTGGAAGAGTTGAATAAAGAGCTTACTGGAATCCAGCAGGGT | |
| | TTTCTGCCCAAGGATTTGCAAGCTGAAGCTCTCTGCAAACTTGATAGGA | |
| | GAGTAAAAGCCACAATAGAGCAGTTTATGAAGATCTTGGAGGAGATTG | |
| | ACACACTGATCCTGCCAGAAAATTTCAAAGACAGTAGATTGAAAAGGA | |
| | AAGGCTTGGTAAAAAAGGTTCAGGCATTCCTAGCCGAGTGTGACACAG | |
| | TGGAGCAGAACATCTGCCAGGAGACTGAGCGGCTGCAGTCTACAAACT | |
| | TTGCCCTGGCCGAGTGAGGTGTAGCAGAAAAAGGCTGTGCTGCCCTGA | |
| | AGAATGGCGCCACCAGCTCTGCCGTCTCTGGAGCGGAATTTACCTGATT | |
| | TCTTCAGGGCTGCTGGGGGCAACTGGCCATTTGCCAATTTTCCTACTCTC | |
| | ACACTGGTTCTCAATGAAAAATAGTGTCTTTGTGATTTTGAGTAAAGCT | |
| | CCTATCTGTTTTCTCCTTCTGTCTCTGTGGTTGTACTGTCCAGCAATCCA | |
| | CCTTTTCTGGAGAGGGCCACCTCTGCCCAAATTTTCCCAGCTGTTTGGAC | |
| | CTCTGGGTGCTTTCTTTGGGCTGGTGAGAGCTCTAATTTGCCTTGGGCCA | |
| | GTTTCAGGTTTATAGGCCCCCTCAGTCTTCAGATACATGAGGGCTTCTTT | |
| | GCTCTTGTGATCGTGTAGTCCCATAGCTGTAAAACCAGAATCACCAGGA | |
| | GGTTGCACCTAGTCAGGAATATTGGGAATGGCCTAGAACAAGGTGTTTG | |
| | GCACATAAGTAGACCACTTATCCCTCATTGTGACCTAATTCCAGAGCAT | |
| | CTGGCTGGGTTGTTGGGTTCTAGACTTTGTCCTCACCTCCCAGTGACCCT | |
| | GACTAGCCACAGGCCATGAGATACCAGGGGGCCGTTCCTTGGATGGAG | |
| | CCTGTGGTTGATGCAAGGCTTCCTTGTCCCCAAGCAAGTCTTCAGAAGG | |
| | TTAGAACCCAGTGTTGACTGAGTCTGTGCTTGAAACCAGGCCAGAGCCA | |
| | TGGATTAGGAAGGGCAAAGAGAAGGCACCAGAATGAGTAAAGCAGGC | |
| | AGGTGGTGAAGCCAACCATAAACTTCTCAGGAGTGACATGTGCTTCCTT | |
| | CAAAGGCATTTTTGTTAACCATATCCTTCTGAGTTCTATGTTTCCTTCAC | |
| | AGCTGTTCTATCCATTTTGTGGACTGTCCCCCACCCCCACCCCATCATTG | |
| | TTTTTAAAAAATTAAGGCCTGGCGCAGCAGCTCATGCCTATAATCCCAG | |
| | CACTTTGGGAGGCTGAGGCGGGCGGATCACTTGAGGCCAGGAGTTTGA | |
| | GACCAGCCCAGGCAACATAGCAAAACCCCATTCTGCTTTAAAAAAAAA | |
| | AAAAAAAAAAATTAGCTTGGCGTAGTGGCATGTGCCTATAATCCCAGCT | |
| | ACTGGGGAGGCTGAGGCACAAGAATCATTTGAACCTGGGAGGTAGAGG | |
| | TTGCTGTGAGCCGAGATTACGCCCCTGCACTCCAGCCTGGGTCACAGAG | |
| | TGAGACTCCATCTCAGAAAAAAAAAAAATTGAGTCAGGTGCAGTAGCT | |
| | CCTTCCTGTAGTCCCAGCTACTTGGGAGGCTGAGGCTAGAGGATCACTT | |
| | GAGCCCAGGAGTTTGAGTCTAGTCTGGGCAACATAGCAAGACCCCATCT | |
| | CTAAAATTTAAGTAAGTAAAAGTAGATAAATAAAAAGAAAAAAAAACT | |
| | GTTTATGTGCTCATCATAAAGTAGAAGAGTGGTTTGCTTTTTTTTTTTTT | |
| | TTTGGATTAATGAGGAAATCATTCTGTGGCTCTAGTCATAATTTATGCTT | |
| | AATAACATTGATAGTAGCCCTTTGCGCTATAACTCTACCTAAAGACTCA | |
| | CATCATTTGGCAGAGAGAGAGTCGTTGAAGTCCCAGGAATTCAGGACT | |
| | GGGCAGGTTAAGACCTCAGACAAGGTAGTAGAGGTAGACTTGTGGACA | |
| | AGGCTCGGGTCCCAGCCCACCGCACCCCAACTTTAATCAGAGTGGTTCA | |
| | CTATTGATCTATTTTTGTGTGATAGCTGTGTGGCGTGGGCCACAACATTT | |
| | AATGAGAAGTTACTGTGCACCAAACTGCCGAACACCATTCTAAACTATT | |
| | CATATATATTAGTCATTTAATTCTTACATAACTTGAGAGGTAGACAGAT | |
| | ATCCTTATTTTAGAGATGAGGAAACCAAGAGAACTTAGGTCATTAGCGC | |
| | AAGGTTGTAGAGTAAGCGGCAAAGCCAAGACACAAAGCTGGGTGGTTT | |
| | GGTTTCAGAGCCAGTGCTTTTCCCCTCTACTGTACTGCCTCTCAACCAAC | |
| | ACAGGGTTGCACAGGCCCATTCTCTGATTTTTTTCCTCTTGTCCTCTGCC | |
| | TCTCCCTCTAGCTCCCACTTCCTCTCTGCTCTAGTTCATTTTCTTTAGAGC | |
| | AGCCCGAGTGATCATGAAGTGCAAATCTTGCCATGTCAGTCCCCTGCTT | |
| | AGAACCCTCCAATGGCTCACTTTCTCTTTAGGCAAAAGTCTTTACCCCAT | |
| | GCCTTCTCCCATCTCATCTCAACCCCCTCATTTGTTGGCTGTCTGCTGTC | |
| | AGCCACTCTTCTTTCAGGTCCTCAGATGCACTGCACCCTCTCCTGCCTGG | |
| | GGGTCTTTGCTCCTGCTACTACCTCTGCTTGAACAGCTCCTCACCTTCCT | |
| | TCCTCCAACCCTACCCTTGTATAGGTGACTTTTGTTCATCCTTCAGAATT | |
| | CAACTCACATGTCTCTTGCATGGAGAACCCTCACCTACTGTGTTGAGAC | |
| | CCTGTCCAGCCCCCAGGTGGGATCCTCTCTCGACTTCCCATACATTTCTT | |
| | TCACAGCATTTACATAGTCCATGATAGTTTACTTGTGGGATTATTTGGTT | |
| | AATCTTTGCCTTTAACACCAGGGTTCCTTGGGTGAAGGAGCTTCTTTATC | |
| | TTGGTAACAGCATTATTTCAAGCATAACTTGTAATATAGTTATATTACAT | |
| | ATATAACATATATATATATAACATAACATATATAACATATATAACAAGC | |
| | ATAACTTGTTATATAGTCTTGTATATAGTAAGACCTCAATAAATATTTG | |
| | GAGAACAAAAAAAAAAAAAAA | |
| NM_000633 | TTTCTGTGAAGCAGAAGTCTGGGAATCGATCTGGAAATCCTCCTAATTT | 151 |
| | TTACTCCCTCTCCCCGCGACTCCTGATTCATTGGGAAGTTTCAAATCAGC | |
| | TATAACTGGAGAGTGCTGAAGATTGATGGGATCGTTGCCTTATGCATTT | |
| | GTTTTGGTTTTACAAAAAGGAAACTTGACAGAGGATCATGCTGTACTTA | |
| | AAAAATACAACATCACAGAGGAAGTAGACTGATATTAACAATACTTAC | |
| | TAATAATAACGTGCCTCATGAAATAAAGATCCGAAAGGAATTGGAATA | |
| | AAAATTTCCTGCATCTCATGCCAAGGGGGAAACACCAGAATCAAGTGTT | |
| | CCGCGTGATTGAAGACACCCCCTCGTCCAAGAATGCAAAGCACATCCA | |
| | ATAAAATAGCTGGATTATAACTCCTCTTCTTTCTCTGGGGGCCGTGGGG | |
| | TGGGAGCTGGGGCGAGAGGTGCCGTTGGCCCCCGTTGCTTTTCCTCTGG | |
| | GAAGGATGGCGCACGCTGGGAGAACAGGGTACGATAACCGGGAGATA | |
| | GTGATGAAGTACATCCATTATAAGCTGTCGCAGAGGGGCTACGAGTGG | |
| | GATGCGGGAGATGTGGGCGCCGCGCCCCCGGGGGCCGCCCCCGCACCG | |
| | GGCATCTTCTCCTCCCAGCCCGGGCACACGCCCCATCCAGCCGCATCCC | |
| | GGGACCCGGTCGCCAGGACCTCGCCGCTGCAGACCCCGGCTGCCCCCG | |
| | GCGCCGCCGCGGGGCCTGCGCTCAGCCCGGTGCCACCTGTGGTCCACCT | |
| | GACCCTCCGCCAGGCCGGCGACGACTTCTCCCGCCGCTACCGCCGCGAC | |
| | TTCGCCGAGATGTCCAGCCAGCTGCACCTGACGCCCTTCACCGCGCGGG | |
| | GACGCTTTGCCACGGTGGTGGAGGAGCTCTTCAGGGACGGGGTGAACT | |
| | GGGGGAGGATTGTGGCCTTCTTTGAGTTCGGTGGGGTCATGTGTGTGGA | |
| | GAGCGTCAACCGGGAGATGTCGCCCCTGGTGGACAACATCGCCCTGTG | |
| | GATGACTGAGTACCTGAACCGGCACCTGCACACCTGGATCCAGGATAA | |
| | CGGAGGCTGGGATGCCTTTGTGGAACTGTACGGCCCCAGCATGCGGCCT | |
| | CTGTTTGATTTCTCCTGGCTGTCTCTGAAGACTCTGCTCAGTTTGGCCCT | |
| | GGTGGGAGCTTGCATCACCCTGGGTGCCTATCTGGGCCACAAGTGAAGT | |
| | CAACATGCCTGCCCCAAACAAATATGCAAAAGGTTCACTAAAGCAGTA | |
| | GAAATAATATGCATTGTCAGTGATGTACCATGAAACAAAGCTGCAGGC | |
| | TGTTTAAGAAAAAATAACACACATATAAACATCACACACACAGACAGA | |
| | CACACACACACACAACAATTAACAGTCTTCAGGCAAAACGTCGAATCA | |
| | GCTATTTACTGCCAAAGGGAAATATCATTTATTTTTTACATTATTAAGAA | |
| | AAAAAGATTTATTTATTTAAGACAGTCCCATCAAAACTCCTGTCTTTGG | |
| | AAATCCGACCACTAATTGCCAAGCACCGCTTCGTGTGGCTCCACCTGGA | |
| | TGTTCTGTGCCTGTAAACATAGATTCGCTTTCCATGTTGTTGGCCGGATC | |
| | ACCATCTGAAGAGCAGACGGATGGAAAAAGGACCTGATCATTGGGGAA | |
| | GCTGGCTTTCTGGCTGCTGGAGGCTGGGGAGAAGGTGTTCATTCACTTG | |
| | CATTTCTTTGCCCTGGGGGCTGTGATATTAACAGAGGGAGGGTTCCTGT | |
| | GGGGGGAAGTCCATGCCTCCCTGGCCTGAAGAAGAGACTCTTTGCATAT | |
| | GACTCACATGATGCATACCTGGTGGGAGGAAAAGAGTTGGGAACTTCA | |
| | GATGGACCTAGTACCCACTGAGATTTCCACGCCGAAGGACAGCGATGG | |
| | GAAAAATGCCCTTAAATCATAGGAAAGTATTTTTTTAAGCTACCAATTG | |
| | TGCCGAGAAAAGCATTTTAGCAATTTATACAATATCATCCAGTACCTTA | |
| | AGCCCTGATTGTGTATATTCATATATTTTGGATACGCACCCCCCAACTCC | |
| | CAATACTGGCTCTGTCTGAGTAAGAAACAGAATCCTCTGGAACTTGAGG | |
| | AAGTGAACATTTCGGTGACTTCCGCATCAGGAAGGCTAGAGTTACCCAG | |
| | AGCATCAGGCCGCCACAAGTGCCTGCTTTTAGGAGACCGAAGTCCGCA | |
| | GAACCTGCCTGTGTCCCAGCTTGGAGGCCTGGTCCTGGAACTGAGCCGG | |
| | GGCCCTCACTGGCCTCCTCCAGGGATGATCAACAGGGCAGTGTGGTCTC | |
| | CGAATGTCTGGAAGCTGATGGAGCTCAGAATTCCACTGTCAAGAAAGA | |
| | GCAGTAGAGGGGTGTGGCTGGGCCTGTCACCCTGGGGCCCTCCAGGTA | |
| | GGCCCGTTTTCACGTGGAGCATGGGAGCCACGACCCTTCTTAAGACATG | |
| | TATCACTGTAGAGGGAAGGAACAGAGGCCCTGGGCCCTTCCTATCAGA | |
| | AGGACATGGTGAAGGCTGGGAACGTGAGGAGAGGCAATGGCCACGGC | |
| | CCATTTTGGCTGTAGCACATGGCACGTTGGCTGTGTGGCCTTGGCCCAC | |
| | CTGTGAGTTTAAAGCAAGGCTTTAAATGACTTTGGAGAGGGTCACAAAT | |
| | CCTAAAAGAAGCATTGAAGTGAGGTGTCATGGATTAATTGACCCCTGTC | |
| | TATGGAATTACATGTAAAACATTATCTTGTCACTGTAGTTTGGTTTTATT | |
| | TGAAAACCTGACAAAAAAAAAGTTCCAGGTGTGGAATATGGGGGTTAT | |
| | CTGTACATCCTGGGGCATTAAAAAAAAAATCAATGGTGGGGAACTATA | |
| | AAGAAGTAACAAAAGAAGTGACATCTTCAGCAAATAAACTAGGAAATT | |
| | TTTTTTTCTTCCAGTTTAGAATCAGCCTTGAAACATTGATGGAATAACTC | |
| | TGTGGCATTATTGCATTATATACCATTTATCTGTATTAACTTTGGAATGT | |
| | ACTCTGTTCAATGTTTAATGCTGTGGTTGATATTTCGAAAGCTGCTTTAA | |
| | AAAAATACATGCATCTCAGCGTTTTTTTGTTTTTAATTGTATTTAGTTAT | |
| | GGCCTATACACTATTTGTGAGCAAAGGTGATCGTTTTCTGTTTGAGATTT | |
| | TTATCTCTTGATTCTTCAAAAGCATTCTGAGAAGGTGAGATAAGCCCTG | |
| | AGTCTCAGCTACCTAAGAAAAACCTGGATGTCACTGGCCACTGAGGAG | |
| | CTTTGTTTCAACCAAGTCATGTGCATTTCCACGTCAACAGAATTGTTTAT | |
| | TGTGACAGTTATATCTGTTGTCCCTTTGACCTTGTTTCTTGAAGGTTTCC | |
| | TCGTCCCTGGGCAATTCCGCATTTAATTCATGGTATTCAGGATTACATGC | |
| | ATGTTTGGTTAAACCCATGAGATTCATTCAGTTAAAAATCCAGATGGCA | |
| | AATGACCAGCAGATTCAAATCTATGGTGGTTTGACCTTTAGAGAGTTGC | |
| | TTTACGTGGCCTGTTTCAACACAGACCCACCCAGAGCCCTCCTGCCCTC | |
| | CTTCCGCGGGGGCTTTCTCATGGCTGTCCTTCAGGGTCTTCCTGAAATGC | |
| | AGTGGTGCTTACGCTCCACCAAGAAAGCAGGAAACCTGTGGTATGAAG | |
| | CCAGACCTCCCCGGCGGGCCTCAGGGAACAGAATGATCAGACCTTTGA | |
| | ATGATTCTAATTTTTAAGCAAAATATTATTTTATGAAAGGTTTACATTGT | |
| | CAAAGTGATGAATATGGAATATCCAATCCTGTGCTGCTATCCTGCCAAA | |
| | ATCATTTTAATGGAGTCAGTTTGCAGTATGCTCCACGTGGTAAGATCCT | |
| | CCAAGCTGCTTTAGAAGTAACAATGAAGAACGTGGACGTTTTTAATATA | |
| | AAGCCTGTTTTGTCTTTTGTTGTTGTTCAAACGGGATTCACAGAGTATTT | |
| | GAAAAATGTATATATATTAAGAGGTCACGGGGGCTAATTGCTGGCTGG | |
| | CTGCCTTTTGCTGTGGGGTTTTGTTACCTGGTTTTAATAACAGTAAATGT | |
| | GCCCAGCCTCTTGGCCCCAGAACTGTACAGTATTGTGGCTGCACTTGCT | |
| | CTAAGAGTAGTTGATGTTGCATTTTCCTTATTGTTAAAAACATGTTAGA | |
| | AGCAATGAATGTATATAAAAGCCTCAACTAGTCATTTTTTTCTCCTCTTC | |
| | TTTTTTTTCATTATATCTAATTATTTTGCAGTTGGGCAACAGAGAACCAT | |
| | CCCTATTTTGTATTGAAGAGGGATTCACATCTGCATCTTAACTGCTCTTT | |
| | ATGAATGAAAAAACAGTCCTCTGTATGTACTCCTCTTTACACTGGCCAG | |
| | GGTCAGAGTTAAATAGAGTATATGCACTTTCCAAATTGGGGACAAGGG | |
| | CTCTAAAAAAAGCCCCAAAAGGAGAAGAACATCTGAGAACCTCCTCGG | |
| | CCCTCCCAGTCCCTCGCTGCACAAATACTCCGCAAGAGAGGCCAGAATG | |
| | ACAGCTGACAGGGTCTATGGCCATCGGGTCGTCTCCGAAGATTTGGCAG | |
| | GGGCAGAAAACTCTGGCAGGCTTAAGATTTGGAATAAAGTCACAGAAT | |
| | TAAGGAAGCACCTCAATTTAGTTCAAACAAGACGCCAACATTCTCTCCA | |
| | CAGCTCACTTACCTCTCTGTGTTCAGATGTGGCCTTCCATTTATATGTGA | |
| | TCTTTGTTTTATTAGTAAATGCTTATCATCTAAAGATGTAGCTCTGGCCC | |
| | AGTGGGAAAAATTAGGAAGTGATTATAAATCGAGAGGAGTTATAATAA | |
| | TCAAGATTAAATGTAAATAATCAGGGCAATCCCAACACATGTCTAGCTT | |
| | TCACCTCCAGGATCTATTGAGTGAACAGAATTGCAAATAGTCTCTATTT | |
| | GTAATTGAACTTATCCTAAAACAAATAGTTTATAAATGTGAACTTAAAC | |
| | TCTAATTAATTCCAACTGTACTTTTAAGGCAGTGGCTGTTTTTAGACTTT | |
| | CTTATCACTTATAGTTAGTAATGTACACCTACTCTATCAGAGAAAAACA | |
| | GGAAAGGCTCGAAATACAAGCCATTCTAAGGAAATTAGGGAGTCAGTT | |
| | GAAATTCTATTCTGATCTTATTCTGTGGTGTCTTTTGCAGCCCAGACAAA | |
| | TGTGGTTACACACTTTTTAAGAAATACAATTCTACATTGTCAAGCTTATG | |
| | AAGGTTCCAATCAGATCTTTATTGTTATTCAATTTGGATCTTTCAGGGAT | |
| | TTTTTTTTTAAATTATTATGGGACAAAGGACATTTGTTGGAGGGGTGGG | |
| | AGGGAGGAAGAATTTTTAAATGTAAAACATTCCCAAGTTTGGATCAGG | |
| | GAGTTGGAAGTTTTCAGAATAACCAGAACTAAGGGTATGAAGGACCTG | |
| | TATTGGGGTCGATGTGATGCCTCTGCGAAGAACCTTGTGTGACAAATGA | |
| | GAAACATTTTGAAGTTTGTGGTACGACCTTTAGATTCCAGAGACATCAG | |
| | CATGGCTCAAAGTGCAGCTCCGTTTGGCAGTGCAATGGTATAAATTTCA | |
| | AGCTGGATATGTCTAATGGGTATTTAAACAATAAATGTGCAGTTTTAAC | |
| | TAACAGGATATTTAATGACAACCTTCTGGTTGGTAGGGACATCTGTTTC | |
| | TAAATGTTTATTATGTACAATACAGAAAAAAATTTTATAAAATTAAGCA | |
| | ATGTGAAACTGAATTGGAGAGTGATAATACAAGTCCTTTAGTCTTACCC | |
| | AGTGAATCATTCTGTTCCATGTCTTTGGACAACCATGACCTTGGACAAT | |
| | CATGAAATATGCATCTCACTGGATGCAAAGAAAATCAGATGGAGCATG | |
| | AATGGTACTGTACCGGTTCATCTGGACTGCCCCAGAAAAATAACTTCAA | |
| | GCAAACATCCTATCAACAACAAGGTTGTTCTGCATACCAAGCTGAGCAC | |
| | AGAAGATGGGAACACTGGTGGAGGATGGAAAGGCTCGCTCAATCAAGA | |
| | AAATTCTGAGACTATTAATAAATAAGACTGTAGTGTAGATACTGAGTAA | |
| | ATCCATGCACCTAAACCTTTTGGAAAATCTGCCGTGGGCCCTCCAGATA | |
| | GCTCATTTCATTAAGTTTTTCCCTCCAAGGTAGAATTTGCAAGAGTGAC | |
| | AGTGGATTGCATTTCTTTTGGGGAAGCTTTCTTTTGGTGGTTTTGTTTAT | |
| | TATACCTTCTTAAGTTTTCAACCAAGGTTTGCTTTTGTTTTGAGTTACTG | |
| | GGGTTATTTTTGTTTTAAATAAAAATAAGTGTACAATAAGTGTTTTTGTA | |
| | TTGAAAGCTTTTGTTATCAAGATTTTCATACTTTTACCTTCCATGGCTCT | |
| | TTTTAAGATTGATACTTTTAAGAGGTGGCTGATATTCTGCAACACTGTA | |
| | CACATAAAAAATACGGTAAGGATACTTTACATGGTTAAGGTAAAGTAA | |
| | GTCTCCAGTTGGCCACCATTAGCTATAATGGCACTTTGTTTGTGTTGTTG | |
| | GAAAAAGTCACATTGCCATTAAACTTTCCTTGTCTGTCTAGTTAATATTG | |
| | TGAAGAAAAATAAAGTACAGTGTGAGATACTG | |
| NM_001012271 | CCCAGAAGGCCGCGGGGGGTGGACCGCCTAAGAGGGCGTGCGCTCCCG | 152 |
| | ACATGCCCCGCGGCGCGCCATTAACCGCCAGATTTGAATCGCGGGACCC | |
| | GTTGGCAGAGGTGGCGGCGGCGGCATGGGTGCCCCGACGTTGCCCCCT | |
| | GCCTGGCAGCCCTTTCTCAAGGACCACCGCATCTCTACATTCAAGAACT | |
| | GGCCCTTCTTGGAGGGCTGCGCCTGCACCCCGGAGCGGATGGCCGAGG | |
| | CTGGCTTCATCCACTGCCCCACTGAGAACGAGCCAGACTTGGCCCAGTG | |
| | TTTCTTCTGCTTCAAGGAGCTGGAAGGCTGGGAGCCAGATGACGACCCC | |
| | ATTGGGCCGGGCACGGTGGCTTACGCCTGTAATACCAGCACTTTGGGAG | |
| | GCCGAGGCGGGCGGATCACGAGAGAGGAACATAAAAAGCATTCGTCCG | |
| | GTTGCGCTTTCCTTTCTGTCAAGAAGCAGTTTGAAGAATTAACCCTTGGT | |
| | GAATTTTTGAAACTGGACAGAGAAAGAGCCAAGAACAAAATTGCAAAG | |
| | GAAACCAACAATAAGAAGAAAGAATTTGAGGAAACTGCGGAGAAAGT | |
| | GCGCCGTGCCATCGAGCAGCTGGCTGCCATGGATTGAGGCCTCTGGCCG | |
| | GAGCTGCCTGGTCCCAGAGTGGCTGCACCACTTCCAGGGTTTATTCCCT | |
| | GGTGCCACCAGCCTTCCTGTGGGCCCCTTAGCAATGTCTTAGGAAAGGA | |
| | GATCAACATTTTCAAATTAGATGTTTCAACTGTGCTCTTGTTTTGTCTTG | |
| | AAAGTGGCACCAGAGGTGCTTCTGCCTGTGCAGCGGGTGCTGCTGGTAA | |
| | CAGTGGCTGCTTCTCTCTCTCTCTCTCTTTTTTGGGGGCTCATTTTTGCTG | |
| | TTTTGATTCCCGGGCTTACCAGGTGAGAAGTGAGGGAGGAAGAAGGCA | |
| | GTGTCCCTTTTGCTAGAGCTGACAGCTTTGTTCGCGTGGGCAGAGCCTT | |
| | CCACAGTGAATGTGTCTGGACCTCATGTTGTTGAGGCTGTCACAGTCCT | |
| | GAGTGTGGACTTGGCAGGTGCCTGTTGAATCTGAGCTGCAGGTTCCTTA | |
| | TCTGTCACACCTGTGCCTCCTCAGAGGACAGTTTTTTTGTTGTTGTGTTT | |
| | TTTTGTTTTTTTTTTTTTGGTAGATGCATGACTTGTGTGTGATGAGAGAA | |
| | TGGAGACAGAGTCCCTGGCTCCTCTACTGTTTAACAACATGGCTTTCTT | |
| | ATTTTGTTTGAATTGTTAATTCACAGAATAGCACAAACTACAATTAAAA | |
| | CTAAGCACAAAGCCATTCTAAGTCATTGGGGAAACGGGGTGAACTTCA | |
| | GGTGGATGAGGAGACAGAATAGAGTGATAGGAAGCGTCTGGCAGATAC | |
| | TCCTTTTGCCACTGCTGTGTGATTAGACAGGCCCAGTGAGCCGCGGGGC | |
| | ACATGCTGGCCGCTCCTCCCTCAGAAAAAGGCAGTGGCCTAAATCCTTT | |
| | TTAAATGACTTGGCTCGATGCTGTGGGGGACTGGCTGGGCTGCTGCAGG | |
| | CCGTGTGTCTGTCAGCCCAACCTTCACATCTGTCACGTTCTCCACACGG | |
| | GGGAGAGACGCAGTCCGCCCAGGTCCCCGCTTTCTTTGGAGGCAGCAG | |
| | CTCCCGCAGGGCTGAAGTCTGGCGTAAGATGATGGATTTGATTCGCCCT | |
| | CCTCCCTGTCATAGAGCTGCAGGGTGGATTGTTACAGCTTCGCTGGAAA | |
| | CCTCTGGAGGTCATCTCGGCTGTTCCTGAGAAATAAAAAGCCTGTCATT | |
| | TCAAACACTGCTGTGGACCCTACTGGGTTTTTAAAATATTGTCAGTTTTT | |
| | CATCGTCGTCCCTAGCCTGCCAACAGCCATCTGCCCAGACAGCCGCAGT | |
| | GAGGATGAGCGTCCTGGCAGAGACGCAGTTGTCTCTGGGCGCTTGCCA | |
| | GAGCCACGAACCCCAGACCTGTTTGTATCATCCGGGCTCCTTCCGGGCA | |
| | GAAACAACTGAAAATGCACTTCAGACCCACTTATTTCTGCCACATCTGA | |
| | GTCGGCCTGAGATAGACTTTTCCCTCTAAACTGGGAGAATATCACAGTG | |
| | GTTTTTGTTAGCAGAAAATGCACTCCAGCCTCTGTACTCATCTAAGCTG | |
| | CTTATTTTTGATATTTGTGTCAGTCTGTAAATGGATACTTCACTTTAATA | |
| | ACTGTTGCTTAGTAATTGGCTTTGTAGAGAAGCTGGAAAAAAATGGTTT | |
| | TGTCTTCAACTCCTTTGCATGCCAGGCGGTGATGTGGATCTCGGCTTCTG | |
| | TGAGCCTGTGCTGTGGGCAGGGCTGAGCTGGAGCCGCCCCTCTCAGCCC | |
| | GCCTGCCACGGCCTTTCCTTAAAGGCCATCCTTAAAACCAGACCCTCAT | |
| | GGCTACCAGCACCTGAAAGCTTCCTCGACATCTGTTAATAAAGCCGTAG | |
| | GCCCTTGTCTAAGTGCAACCGCCTAGACTTTCTTTCAGATACATGTCCAC | |
| | ATGTCCATTTTTCAGGTTCTCTAAGTTGGAGTGGAGTCTGGGAAGGGTT | |
| | GTGAATGAGGCTTCTGGGCTATGGGTGAGGTTCCAATGGCAGGTTAGA | |
| | GCCCCTCGGGCCAACTGCCATCCTGGAAAGTAGAGACAGCAGTGCCCG | |
| | CTGCCCAGAAGAGACCAGCAAGCCAAACTGGAGCCCCCATTGCAGGCT | |
| | GTCGCCATGTGGAAAGAGTAACTCACAATTGCCAATAAAGTCTCATGTG | |
| | GTTTTATCTAAAAAAAAAAAAAAAAAAAAAAAAA | |
| BX647539 | AATGAGGGTATTTATAAACTACTTAAATTATAAAAAGAATGAGACATC | 153 |
| | AGACTTACAGTTTTGGATACTAATTTTTTTCACTTAACGTTCATTATGTG | |
| | ATAGGAGTTTTCCATCCTATTATACCGCTGTGCGATCTGATCTTGGGCAC | |
| | GTTAACCAACCTCTTGTTGCCTCGATTTTCTCACCTGTAAAAGTGGGGGT | |
| | AATCATAATGCTTACTTAGTAGGATAGCCCTGAAGAATAAGTGACTTAG | |
| | CGAACATAAATAGCTTACAATAGGGTTTTCAGCATGGGAAGGATTCAGT | |
| | AAATGTTAGCTGTCATCATCACCACCTACAAAGGAAGCAATACTGTGCT | |
| | GAAAGTTTTTCCATCATTAATGTAATTTCTATAGTACGATTCCCAAGAA | |
| | GATATTAAAATTATGGAAATAAAGGTATTGGTATATTCCTAATTATTTC | |
| | CTAAAAGATTGTATTGATAAATATGCTCATCCTTCCCTTAACGGGATGC | |
| | ATTCCAGAAAAACAAGTCAAATGTTAGACAAAGTATCAGAAGGGAAAT | |
| | TCTGTAGCCAGAGAGCTAAAAATTACAATAGGGTCTCTAATTATACTTC | |
| | AACTTTTTTAGGAATAATTCTCAGTGTGTTTTCCCACATTTCATATGTAA | |
| | TTTTTTTTTTTTTTTTTTTTTGAGACAGAGCCTCGCCCTGTCACCAGGCTG | |
| | GAGTACAGTGGCGCGATCTCGGCTCACTGCAACTTCCACCTGCTGGGTT | |
| | CAAGCAATTCTTCTGACCTCAGGTGATCCACCCGCCTCGGCCTCCCAAA | |
| | GTGCTGGGATTATAACAGGCGTGGCATGAGTCACCGCGCCCGGCCGAT | |
| | CTTTACTTTTTTATTCTTTGTACCCCCTGCCTATCCAGTTAGCATGTGATT | |
| | AAAGTCAAAGATTTGCCACTTTGGGCCACATCTATTAATTTTCATCTTTG | |
| | TTATAATTGTATTTAGTTTTTGATCTACACTGCTTATTACTCCCAGTCATT | |
| | TTTTATAGAACTGAAAATCTGGTAAAATACTCAAAATTGCACTGACTTC | |
| | TATGTAGAGGCGACACTCCATCAGAACCGTGGGCTGACAGGGAATCCC | |
| | ACTGTGCAGGAGCTGCGCGCATTTTCATTTCTGATTCTCTTTGGCGTATC | |
| | CAGGACTCTGATGACATGATCATATATTTATCAGTAGTAACAGGTTGGG | |
| | CCATTTGTTTTTTGTGGTAAATCATATATTTAAGATTTTAGAAATAAGTT | |
| | GATAGCCATGTATTTTGGAATTTGAAAAAGACATTGCATTACTCAGCTT | |
| | CAAATTAAGCTTTAATCAAATAGTGAAACTTTCCATTAATGGACAGTGT | |
| | ATACCTTTTTGTGTATTTAAAAAAAAAAACACTGAATATAGTGCCTTTG | |
| | TGACAGGGGAGCTTGGTTCCTGACAATGTCCTCTTGAGCCTTTTTTTTTT | |
| | TTTTGAGATGGAGTCTCACTGTGTCACCCAGGCTGGAGTGCAGTGGCGC | |
| | CATCTTGGCTCACTGCAACCTCCGCCCCCTGGGTTCAAGTGATTCTCATT | |
| | CCTCAGCTTCCTAAGTAGCTGGGATTACAGGCACGCACCACCATGACCA | |
| | GCTAATTTTTATACTTTTAGTAGAGACAGGGTTTTGCCATGTTGGCTAGG | |
| | TTGGTCTCGAACTCCTGACCTCAAGTAATCCACCCACCATGGCCTCCCC | |
| | AAAGTGCTGGGATTACAGGCGTGAGCCATTTCACCCGGCCTCTCTTCCG | |
| | TCTTTGAGCTGTGAGGAAATAGCTACATTACATGAGCTGCTAGATCTGC | |
| | CTTATGGTCAGAAATGAAGGTTGAACTCTCAGGAACAGTGACATATATA | |
| | CACACTGATATTTCCAAAGTACAATGCCCCAAATTGATCCACAAAGGAA | |
| | TTAAGGTCATTTGCAACAAAATCACAGAATAGTAACAAATAAATAGAA | |
| | GATAAATATGGCCAGGGATGCTGCAAACTGATATACTGCCAAGTTTATC | |
| | AGTTGGGAATCCCAACAGTGAAAAGCATAAAAATGAAAGGAATTTTAA | |
| | GGAGACTTTTTATAGAAGAGTGGGAAGGATTGGAGGAGCCAACAAGTG | |
| | ATGGTGAGGCACACAGGGAAGAGCTTCAGTGGGCACCATCCCCTCTCT | |
| | GGTTTGAAGGGGTAGGGAGGGGACCAGAGCTGGGAGGAGGGGGCTGG | |
| | AATACTGCTGGAGGAGCCACTCCCTTCCAGACCTGCTGTGGCCATCACA | |
| | GAATGCAGCCACTGCCAGAGCAGCAGCCCGAGGAACCAGGCAGGGGG | |
| | AGCACAAGTACCCTAGCCTCTCTCTTTCTGTTTCTTGCCTGCCGATCTCC | |
| | TCCACTGGCTAAACCCAGCTGGATGCTAAGAGTACAGTCAGCCTGCCTG | |
| | CTGAGGAGGGACCACCAGGGACCACCATCAGCAAGGGATCCAATGTCT | |
| | TTCTGCCTCTGCAGAATGAAGGTTGGGGCGCGGGGGGCGCTCTACTTCT | |
| | TAGGGATATTGTGGGAATAAAAGGAAATAGGCAAAAAATGTTTTTGAA | |
| | AAACAAAGCACATACTGCGCACCCGTGGGCCACTACTGCTTTTGACCCC | |
| | TGGCTCTGTTTCATGAAGTAATGTCGTGTCATTCTCTTTTTAGGTGCTAC | |
| | AGGATTTCTTTAGGTTTGTTTTCTGTCCACCATATTTCAACTCATGTGTG | |
| | CTGTTTGTTGTGCTAAAACAAATATTTGCTGATGCCTGAGTGAATAGTT | |
| | GAATATTTTATATAAGTCAAATTTATACGTAATGATTTTTCTTGTAACTT | |
| | AGCCGTTTCTCTTTTACAAACTCAGAAAACCTCAGACTTTGAAAAGGCC | |
| | TTGAAGTTCCTCACCTGAAATCTGAGAACTTGGAGCGCCTTAAAAAATC | |
| | TAAAGGAAAACAAAACAGTGAAAGAACATGATATAGTCAGTGTAGAGA | |
| | ATAAAATTATTTATGTAATTAATATTGAGGATGCAGATAACACATTGTG | |
| | AAATCTTGCTTGTAAAAAATCTCGATCTGCTGAAGAAAGATGTTCTCTC | |
| | TAGAGATCTTTGAAAGCATAATTATTGAGCTTTTAAAATGTTAGAAACA | |
| | AAAGTTAGACCCACACATATTCTGGCGTGTGGAAGATTTGCATTCCTTC | |
| | CCCTGCCCGCCCCGCCCCCACACTTGTGAGTTGTGCCTGTGTACGCAGT | |
| | TCCTGTAGCACTCGGCTGGGCAGAAATCATCTTTCAGCACTAAGGGAAC | |
| | ATAGTTATGATCTGGACCTTCTGGGAGTGGTCAGTGCCCAAGAACAGGT | |
| | ATGGGACTCCAGAAAGTTCTGCTCTCAACCCTATTTTGAAATAGAGTTA | |
| | CACATTGTTCTACAATTATTTGAGTTAATAAGCAGCTCTTTTCAAACGTG | |
| | ATTATGCCCTTCCAAGTTTAAATACACTAGACTTTAGTGAAAGTAATTG | |
| | ACCTCATCTCATTTCTCTCCTGTTATATTAAGATCACTTTCAGTAAAAGG | |
| | TAGAAGCTTTTGAAGTGGTGAGGAGGAGGTAGAGGAGGGACATAGAGC | |
| | AGATAGGGGCTGGAAAGTGGGGTGAGGAAGAGAGTGGCTTCTCTTTGG | |
| | CAGAGTACCAAGGAAAAGCCCTATCTGTACAGAACCTTTGTGCCTGGG | |
| | AACTTGATGGCTGCAACCTGAGCCTCAACCTAGTTTGCTTGCGGAGCCA | |
| | GAAGAGAAGCTAAAAACCTTCAGTTAACCAAGCCAGACACCAAGAAAG | |
| | TTAAACCGAAAGAGAACCCCCCACCCCCCGCAAAAAAAAGAAGTAAAG | |
| | TGGGTTAAAGTGATATCATGTTAGCACAGAAAGAGAACATAAGGGTCA | |
| | TCTAAGTTCATCTGCCCCCTCTTCTATTTCAAGGTGCAGAAACTAAGGC | |
| | ACAAGGGACCCCGTGTCCTGCTCTTGATCACATAGCTAGTGGGTGCCAA | |
| | GCCAGGTCTAGAACTCTGTTCTCTGGGGTCACAGGCTGGCTCTTCATCC | |
| | CTCTAGAGAGATAGCTCATCTGTGTGCACCTGAGCCCGTTGTGTTTCGG | |
| | AGTCAAAGCAAATAAAGGCTCAAACTCCAAGACTGTTTTGCAGACCGG | |
| | CTGCAGTAGATATGGGGGGAGGAGAAACCTGCTTTAAATTGCTTCAAG | |
| | CAAGTTGTTTCTGCAAAGGTGTTGACTTTTTTCTTTCAACTTTCTAGTGA | |
| | GTCACTGCAGCCTGAGCTGTTATTTGTCATTATGCAATAATTCAGGAAC | |
| | TAACTCAAGATTCTTCTTTTTAAATTATTTGTTTATTTAGAGACAGAGTC | |
| | TTGCTCTGTTGCCCAGGCTGGAGTGCAGTGGTGTGATCTCGGCTCACTG | |
| | CAGCCTCTGCCTCCTGGGTTCAAGCAATTCTCATGTCTCAGCCTCCCGA | |
| | ATAGCTGGTATTGCAGGCTCGTGCCACCACCCCCTGCTAATTTTTGTAAT | |
| | TTTAGTGGAGACACGGTTTCGCCATGTTGGCCGGGCTCGTCTTGAGCTC | |
| | CTGGCCTCAGGTGATCCGCCCGCCTCGGCCTCCCAAAGTGCTGGGATTG | |
| | CAGCCGTGAGCCTCCACACCCGGCCTATTTATTTATTTTTAAATTGGCTG | |
| | CTCTTAGAAAGGCATACCATGTTTCTGGATGGGAAGGCTTATTAATTCA | |
| | CCCTAATTTAATGTATAAATTTGATGCAATCATAGTCACAGTCCCAGTG | |
| | GAATTTTTTAACTTGGTAAGATGTTCTAAAATTAATGAGAGAACTTGAA | |
| | TTACCAGGTATTGAAACACTGTAAAGCCACAATCATGTAAACAGTATGT | |
| | TATAACCATGGGAATAGAGGTCTGTGATACAGCAGAAAAAAGTGAAAA | |
| | AAAGAATAACTGTATTCATAAAAATTTAAATGTGGAGTCACTGGGGGA | |
| | AAGGATTAAATATTCGATAATGTAGAAACAACTCAACTATTTGGAGAA | |
| | ATGTAAATTTAGAGCCTTATCTCATGCCATATACCAAAATACTATTTAG | |
| | ATTTGATTAAAAAATAAAAAAAAAAAAAAAAAAA | |
| NM_031966 | CGAACGCCTTCGCGCGATCGCCCTGGAAACGCATTCTCTGCGACCGGCA | 154 |
| | GCCGCCAATGGGAAGGGAGTGAGTGCCACGAACAGGCCAATAAGGAG | |
| | GGAGCAGTGCGGGGTTTAAATCTGAGGCTAGGCTGGCTCTTCTCGGCGT | |
| | GCTGCGGCGGAACGGCTGTTGGTTTCTGCTGGGTGTAGGTCCTTGGCTG | |
| | GTCGGGCCTCCGGTGTTCTGCTTCTCCCCGCTGAGCTGCTGCCTGGTGA | |
| | AGAGGAAGCCATGGCGCTCCGAGTCACCAGGAACTCGAAAATTAATGC | |
| | TGAAAATAAGGCGAAGATCAACATGGCAGGCGCAAAGCGCGTTCCTAC | |
| | GGCCCCTGCTGCAACCTCCAAGCCCGGACTGAGGCCAAGAACAGCTCTT | |
| | GGGGACATTGGTAACAAAGTCAGTGAACAACTGCAGGCCAAAATGCCT | |
| | ATGAAGAAGGAAGCAAAACCTTCAGCTACTGGAAAAGTCATTGATAAA | |
| | AAACTACCAAAACCTCTTGAAAAGGTACCTATGCTGGTGCCAGTGCCAG | |
| | TGTCTGAGCCAGTGCCAGAGCCAGAACCTGAGCCAGAACCTGAGCCTG | |
| | TTAAAGAAGAAAAACTTTCGCCTGAGCCTATTTTGGTTGATACTGCCTC | |
| | TCCAAGCCCAATGGAAACATCTGGATGTGCCCCTGCAGAAGAAGACCT | |
| | GTGTCAGGCTTTCTCTGATGTAATTCTTGCAGTAAATGATGTGGATGCA | |
| | GAAGATGGAGCTGATCCAAACCTTTGTAGTGAATATGTGAAAGATATTT | |
| | ATGCTTATCTGAGACAACTTGAGGAAGAGCAAGCAGTCAGACCAAAAT | |
| | ACCTACTGGGTCGGGAAGTCACTGGAAACATGAGAGCCATCCTAATTG | |
| | ACTGGCTAGTACAGGTTCAAATGAAATTCAGGTTGTTGCAGGAGACCAT | |
| | GTACATGACTGTCTCCATTATTGATCGGTTCATGCAGAATAATTGTGTG | |
| | CCCAAGAAGATGCTGCAGCTGGTTGGTGTCACTGCCATGTTTATTGCAA | |
| | GCAAATATGAAGAAATGTACCCTCCAGAAATTGGTGACTTTGCTTTTGT | |
| | GACTGACAACACTTATACTAAGCACCAAATCAGACAGATGGAAATGAA | |
| | GATTCTAAGAGCTTTAAACTTTGGTCTGGGTCGGCCTCTACCTTTGCACT | |
| | TCCTTCGGAGAGCATCTAAGATTGGAGAGGTTGATGTCGAGCAACATAC | |
| | TTTGGCCAAATACCTGATGGAACTAACTATGTTGGACTATGACATGGTG | |
| | CACTTTCCTCCTTCTCAAATTGCAGCAGGAGCTTTTTGCTTAGCACTGAA | |
| | AATTCTGGATAATGGTGAATGGACACCAACTCTACAACATTACCTGTCA | |
| | TATACTGAAGAATCTCTTCTTCCAGTTATGCAGCACCTGGCTAAGAATG | |
| | TAGTCATGGTAAATCAAGGACTTACAAAGCACATGACTGTCAAGAACA | |
| | AGTATGCCACATCGAAGCATGCTAAGATCAGCACTCTACCACAGCTGA | |
| | ATTCTGCACTAGTTCAAGATTTAGCCAAGGCTGTGGCAAAGGTGTAACT | |
| | TGTAAACTTGAGTTGGAGTACTATATTTACAAATAAAATTGGCACCATG | |
| | TGCCATCTGTACATATTACTGTTGCATTTACTTTTAATAAAGCTTGTGGC | |
| | CCCTTTTACTTTTTTATAGCTTAACTAATTTGAATGTGGTTACTTCCTACT | |
| | GTAGGGTAGCGGAAAAGTTGTCTTAAAAGGTATGGTGGGGATATTTTTA | |
| | AAAACTCCTTTTGGTTTACCTGGGGATCCAATTGATGTATATGTTTATAT | |
| | ACTGGGTTCTTGTTTTATATACCTGGCTTTTACTTTATTAATATGAGTTA | |
| | CTGAAGGTGATGGAGGTATTTGAAAATTTTACTTCCATAGGACATACTG | |
| | CATGTAAGCCAAGTCATGGAGAATCTGCTGCATAGCTCTATTTTAAAGT | |
| | AAAAGTCTACCACCGAATCCCTAGTCCCCCTGTTTTCTGTTTCTTCTTGT | |
| | GATTGCTGCCATAATTCTAAGTTATTTACTTTTACCACTATTTAAGTTAT | |
| | CAACTTTAGCTAGTATCTTCAAACTTTCACTTTGAAAAATGAGAATTTTA | |
| | TATTCTAAGCCAGTTTTCATTTTGGTTTTGTGTTTTGGTTAATAAAACAA | |
| | TACTCAAATACAAAAAAAAAAAA | |
| BC035498 | GCGGCCGCCAGCGCGGTGTAGGGGGCAGGCGCGGATCCCGCCACCGCC | 155 |
| | GCGCGCTCGGCCCGCCGACTCCCGGCGCCGCCGCCGCCACTGCCGTCGC | |
| | CGCCGCCGCCTGCCGGGACTGGAGCGCGCCGTCCGCCGCGGACAAGAC | |
| | CCTGGCCTCAGGCCGGAGCAGCCCCATCATGCCGAGGGAGCGCAGGGA | |
| | GCGGGATGCGAAGGAGCGGGACACCATGAAGGAGGACGGCGGCGCGG | |
| | AGTTCTCGGCTCGCTCCAGGAAGAGGAAGGCAAACGTGACCGTTTTTTT | |
| | GCAGGATCCAGATGAAGAAATGGCCAAAATCGACAGGACGGCGAGGG | |
| | ACCAGTGTGGGAGCCAGCCTTGGGACAATAATGCAGTCTGTGCAGACC | |
| | CCTGCTCCCTGATCCCCACACCTGACAAAGAAGATGATGACCGGGTTTA | |
| | CCCAAACTCAACGTGCAAGCCTCGGATTATTGCACCATCCAGAGGCTCC | |
| | CCGCTGCCTGTACTGAGCTGGGCAAATAGAGAGGAAGTCTGGAAAATC | |
| | ATGTTAAACAAGGAAAAGACATACTTAAGGGATCAGCACTTTCTTGAG | |
| | CAACACCCTCTTCTGCAGCCAAAAATGCGAGCAATTCTTCTGGATTGGT | |
| | TAATGGAGGTGTGTGAAGTCTATAAACTTCACAGGGAGACCTTTTACTT | |
| | GGCACAAGATTTCTTTGACCGGTATATGGCGACACAAGAAAATGTTGTA | |
| | AAAACTCTTTTACAGCTTATTGGGATTTCATCTTTATTTATTGCAGCCAA | |
| | ACTTGAGGAAATCTATCCTCCAAAGTTGCACCAGTTTGCGTATGTGACA | |
| | GATGGAGCTTGTTCAGGAGATGAAATTCTCACCATGGAATTAATGATTA | |
| | TGAAGGCCCTTAAGTGGCGTTTAAGTCCCCTGACTATTGTGTCCTGGCT | |
| | GAATGTATACATGCAGGTTGCATATCTAAATGACTTACATGAAGTGCTA | |
| | CTGCCGCAGTATCCCCAGCAAATCTTTATACAGATTGCAGAGCTGTTGG | |
| | ATCTCTGTGTCCTGGATGTTGACTGCCTTGAATTTCCTTATGGTATACTT | |
| | GCTGCTTCGGCCTTGTATCATTTCTCGTCATCTGAATTGATGCAAAAGGT | |
| | TTCAGGGTATCAGTGGTGCGACATAGAGAACTGTGTCAAGTGGATGGTT | |
| | CCATTTGCCATGGTTATAAGGGAGACGGGGAGCTCAAAACTGAAGCAC | |
| | TTCAGGGGCGTCGCTGATGAAGATGCACACAACATACAGACCCACAGA | |
| | GACAGCTTGGATTTGCTGGACAAAGCCCGAGCAAAGAAAGCCATGTTG | |
| | TCTGAACAAAATAGGGCTTCTCCTCTCCCCAGTGGGCTCCTCACCCCGC | |
| | CACAGAGCGGTAAGAAGCAGAGCAGCGGGCCGGAAATGGCGTGACCA | |
| | CCCCATCCTTCTCCACCAAAGACAGTTGCGCGCCTGCTCCACGTTCTCTT | |
| | CTGTCTGTTGCAGCGGAGGCGTGCGTTTGCTTTTACAGATATCTGAATG | |
| | GAAGAGTGTTTCTTCCACAACAGAAGTATTTCTGTGGATGGCATCAAAC | |
| | AGGGCAAAGTGTTTTTTATTGAATGCTTATAGGTTTTTTTTAAATAAGTG | |
| | GGTCAAGTACACCAGCCACCTCCAGACACCAGTGCGTGCTCCCGATGCT | |
| | GCTATGGAAGGTGCTACTTGACCTAAGGGACTCCCACAACAACAAAAG | |
| | CTTGAAGCTGTGGAGGGCCACGGTGGCGTGGCTCTCCTCGCAGGTGTTC | |
| | TGGGCTCCGTTGTACCAAGTGGAGCAGGTGGTTGCGGGCAAGCGTTGTG | |
| | CAGAGCCCATAGCCAGCTGGGCAGGGGGCTGCCCTCTCCACATTATCAG | |
| | TTGACAGTGTACAATGCCTTTGATGAACTGTTTTGTAAGTGCTGCTATAT | |
| | CTATCCATTTTTTAATAAAGATAATACTGTTTTTGAAAAAAAAAAAAAA | |
| | AAAAAAAAAAAAAAAAAAAAAAAAAAAAAAAAAAAAAAAAAAAAAA | |
| | AAAAAAAAAAAAAAA | |
| BG256659 | GAGGGCACGGGCTCCGTAGGCACCAACTGCAAGGACCCCTCCCCCTGC | 156 |
| | GGGCGCTCCCATGGCACAGTTCGCGTTCGAGAGTGACCTGCACTCGCTG | |
| | CTTCAGCTGGATGCACCCATCCCCAATGCACCCCCTGCGCGCTGGCAGC | |
| | GCAAAGCCAAGGAAGCCGCAGGCCCGGCCCCCTCACCCATGCGGGCCG | |
| | CCAACCGATCCCACAGCGCCGGCAGGACTCCGGGCCGAACTCCTGGCA | |
| | AATCCAGTTCCAAGGTTCAGACCACTCCTAGCAAACCTGGCGGTGACCG | |
| | CTATATCCCCCATCGCAGTGCTGCCCAGATGGAGGTGGCCAGCTTCCTC | |
| | CTGAGCAAGGAGAACCAGCCTGAAAACAGCCAGACGCCCACCAAGAA | |
| | GGAACATCAGAAAGCCTGGGCTTTGAACCTGAACGGTTTTGATGTAGA | |
| | GGAAGCCAAGATCCTTCGGCTCAGTGGAAAAACCACAAAAATGCGCCA | |
| | GAGGGTTATCACGAACAGACTGAAAGTACTCTACAGCCAAAAGGCCAC | |
| | TCCTGGCTCCAGCCGGAAGACCTGCCGTTTACATTCCTTCCCTGCCAAG | |
| | ACCGTATCCTGGATGCGCCTGAAATCGAATGACTATTAACTGAACCTGT | |
| | GGGACTGGCAGTCCGGGGAATGTCCGGGCCGGGCCACGGCCACGAGGT | |
| | GTTCCGTGTGGAGTGCAAGCTGGGACACACCGTGCCGCTTGTGCACAGG | |
| | GCCACGCGGGGAAATAATCCCGGGGCGCGCAAAGCGGCACTGGCGAGA | |
| | GCCGCACGGGCCGGTGCTGGGGGTGGTACAACAGGCCAAAACAACACA | |
| | CAAGGCCAACAAGACATACGCGCGCTGACACCACGGTGCAAAGCGCTC | |
| | AGACGAGTAGTAACCGGCACTGTGGTTGCTGCCTCCCCACCTCTCCCGC | |
| | TCTCAGCGTAAGATAAAAGAAAGAAGAGCAAAAAGCAAAGAAAGAAG | |
| | ACGAGACGAGACACACAGGAACGAACAGTAAAGCAAGCTAAAGCAAA | |
| | CGCAAGACCAGACAACAGAAATAGAAAGAACCAACAGAGAGGAGACA | |
| | GAACAGGACGCCAGCAACATAGCAACAAACGAACAGAAGAGAGCACT | |
| | AAACAAAAGCAGCAGCAAGACGAGACAGGAGAGAAGGAGGAAGGAG | |
| | GGCCGAGCGAGCAGGGAGCGCGAGCAGCGAGGCGAAGCAGCAGACAA | |
| | GGGCAGGCGAAGGGCAACGAGAGGAGGCACCACACAAAAAGGAGAGG | |
| | GGACAGGAGAAGCAGCGAGAGAAGCGGAGGAGCAACAAGAGGAAGA | |
| | AAAGGAGAGGGAGAGGAGGGAGAGAGCGGAAGGAGGAAGAAACAGC | |
| | ACGAGGCGACGAAGGGGGGAGACGCGGGGGCAGGAAAAGACACAGGA | |
| | AGGCAGCGCGGAGGAGGAGAAGGGGAAGCAGGAAGGAGACGGAAGG | |
| | AGAAGAGGGAGAGGACAGCGCAAGAGAGCGCGCGCGGCGACAGCGAG | |
| | GGACGGAGCGAGAGAGAGGAAACGGAAAGCGAGAGGGAAGAGGAGA | |
| | GGCAACGCAGCGAACCAACCGAAAACAGCAGAAAGAGAGGAGAAGGA | |
| | CGCGCAAAGAGGCAAGCGCAAGACGACAGGAAACGAAGCGAGAGACG | |
| | AGAAGCCGGTGACGAGCAGGAGAAAGGGAAGGCAGGAGACAGGACAG | |
| | GCGGAAGAGAGACACGCGAGACGCAAAGAGTGAGCAGAACGAAGCGA | |
| | AGAGCAACGCACGAGAGAAACGAC | |
| NM_001254 | GAGCGCGGCTGGAGTTTGCTGCTGCCGCTGTGCAGTTTGTTCAGGGGCT | 157 |
| | TGTGGTGGTGAGTCCGAGAGGCTGCGTGTGAGAGACGTGAGAAGGATC | |
| | CTGCACTGAGGAGGTGGAAAGAAGAGGATTGCTCGAGGAGGCCTGGGG | |
| | TCTGTGAGGCAGCGGAGCTGGGTGAAGGCTGCGGGTTCCGGCGAGGCC | |
| | TGAGCTGTGCTGTCGTCATGCCTCAAACCCGATCCCAGGCACAGGCTAC | |
| | AATCAGTTTTCCAAAAAGGAAGCTGTCTCGGGCATTGAACAAAGCTAA | |
| | AAACTCCAGTGATGCCAAACTAGAACCAACAAATGTCCAAACCGTAAC | |
| | CTGTTCTCCTCGTGTAAAAGCCCTGCCTCTCAGCCCCAGGAAACGTCTG | |
| | GGCGATGACAACCTATGCAACACTCCCCATTTACCTCCTTGTTCTCCACC | |
| | AAAGCAAGGCAAGAAAGAGAATGGTCCCCCTCACTCACATACACTTAA | |
| | GGGACGAAGATTGGTATTTGACAATCAGCTGACAATTAAGTCTCCTAGC | |
| | AAAAGAGAACTAGCCAAAGTTCACCAAAACAAAATACTTTCTTCAGTT | |
| | AGAAAAAGTCAAGAGATCACAACAAATTCTGAGCAGAGATGTCCACTG | |
| | AAGAAAGAATCTGCATGTGTGAGACTATTCAAGCAAGAAGGCACTTGC | |
| | TACCAGCAAGCAAAGCTGGTCCTGAACACAGCTGTCCCAGATCGGCTG | |
| | CCTGCCAGGGAAAGGGAGATGGATGTCATCAGGAATTTCTTGAGGGAA | |
| | CACATCTGTGGGAAAAAAGCTGGAAGCCTTTACCTTTCTGGTGCTCCTG | |
| | GAACTGGAAAAACTGCCTGCTTAAGCCGGATTCTGCAAGACCTCAAGA | |
| | AGGAACTGAAAGGCTTTAAAACTATCATGCTGAATTGCATGTCCTTGAG | |
| | GACTGCCCAGGCTGTATTCCCAGCTATTGCTCAGGAGATTTGTCAGGAA | |
| | GAGGTATCCAGGCCAGCTGGGAAGGACATGATGAGGAAATTGGAAAAA | |
| | CATATGACTGCAGAGAAGGGCCCCATGATTGTGTTGGTATTGGACGAG | |
| | ATGGATCAACTGGACAGCAAAGGCCAGGATGTATTGTACACGCTATTTG | |
| | AATGGCCATGGCTAAGCAATTCTCACTTGGTGCTGATTGGTATTGCTAA | |
| | TACCCTGGATCTCACAGATAGAATTCTACCTAGGCTTCAAGCTAGAGAA | |
| | AAATGTAAGCCACAGCTGTTGAACTTCCCACCTTATACCAGAAATCAGA | |
| | TAGTCACTATTTTGCAAGATCGACTTAATCAGGTATCTAGAGATCAGGT | |
| | TCTGGACAATGCTGCAGTTCAATTCTGTGCCCGCAAAGTCTCTGCTGTTT | |
| | CAGGAGATGTTCGCAAAGCACTGGATGTTTGCAGGAGAGCTATTGAAA | |
| | TTGTAGAGTCAGATGTCAAAAGCCAGACTATTCTCAAACCACTGTCTGA | |
| | ATGTAAATCACCTTCTGAGCCTCTGATTCCCAAGAGGGTTGGTCTTATTC | |
| | ACATATCCCAAGTCATCTCAGAAGTTGATGGTAACAGGATGACCTTGAG | |
| | CCAAGAAGGAGCACAAGATTCCTTCCCTCTTCAGCAGAAGATCTTGGTT | |
| | TGCTCTTTGATGCTCTTGATCAGGCAGTTGAAAATCAAAGAGGTCACTC | |
| | TGGGGAAGTTATATGAAGCCTACAGTAAAGTCTGTCGCAAACAGCAGG | |
| | TGGCGGCTGTGGACCAGTCAGAGTGTTTGTCACTTTCAGGGCTCTTGGA | |
| | AGCCAGGGGCATTTTAGGATTAAAGAGAAACAAGGAAACCCGTTTGAC | |
| | AAAGGTGTTTTTCAAGATTGAAGAGAAAGAAATAGAACATGCTCTGAA | |
| | AGATAAAGCTTTAATTGGAAATATCTTAGCTACTGGATTGCCTTAAATT | |
| | CTTCTCTTACACCCCACCCGAAAGTATTCAGCTGGCATTTAGAGAGCTA | |
| | CAGTCTTCATTTTAGTGCTTTACACATTCGGGCCTGAAAACAAATATGA | |
| | CCTTTTTTACTTGAAGCCAATGAATTTTAATCTATAGATTCTTTAATATT | |
| | AGCACAGAATAATATCTTTGGGTCTTACTATTTTTACCCATAAAAGTGA | |
| | CCAGGTAGACCCTTTTTAATTACATTCACTACTTCTACCACTTGTGTATC | |
| | TCTAGCCAATGTGCTTGCAAGTGTACAGATCTGTGTAGAGGAATGTGTG | |
| | TATATTTACCTCTTCGTTTGCTCAAACATGAGTGGGTATTTTTTTGTTTGT | |
| | TTTTTTTGTTGTTGTTGTTTTTGAGGCGCGTCTCACCCTGTTGCCCAGGCT | |
| | GGAGTGCAATGGCGCGTTCTCTGCTCACTACAGCACCCGCTTCCCAGGT | |
| | TGAAGTGATTCTCTTGCCTCAGCCTCCCGAGTAGCTGGGATTACAGGTG | |
| | CCCACCACCGCGCCCAGCTAATTTTTTAATTTTTAGTAGAGACAGGGTT | |
| | TTACCATGTTGGCCAGGCTGGTCTTGAACTCCTGACCCTCAAGTGATCT | |
| | GCCCACCTTGGCCTCCCTAAGTGCTGGGATTATAGGCGTGAGCCACCAT | |
| | GCTCAGCCATTAAGGTATTTTGTTAAGAACTTTAAGTTTAGGGTAAGAA | |
| | GAATGAAAATGATCCAGAAAAATGCAAGCAAGTCCACATGGAGATTTG | |
| | GAGGACACTGGTTAAAGAATTTATTTCTTTGTATAGTATACTATGTTCAT | |
| | GGTGCAGATACTACAACATTGTGGCATTTTAGACTCGTTGAGTTTCTTG | |
| | GGCACTCCCAAGGGCGTTGGGGTCATAAGGAGACTATAACTCTACAGA | |
| | TTGTGAATATATTTATTTCAAGTTGCATTCTTTGTCTTTTTAAGCAATC | |
| | AGATTTCAAGAGAGCTCAAGCTTTCAGAAGTCAATGTGAAAATTCCTTC | |
| | CTAGGCTGTCCCACAGTCTTTGCTGCCCTTAGATGAAGCCACTTGTTTCA | |
| | AGATGACTACTTTGGGGTTGGGTTTTCATCTAAACACATTTTTCCAGTCT | |
| | TATTAGATAAATTAGTCCATATGGTTGGTTAATCAAGAGCCTTCTGGGT | |
| | TTGGTTTGGTGGCATTAAATGG | |
| NM_031423 | GCGGAATGGGGCGGGACTTCCAGTAGGAGGCGGCAAGTTTGAAAAGTG | 158 |
| | ATGACGGTTGACGTTTGCTGATTTTTGACTTTGCTTGTAGCTGCTCCCCG | |
| | AACTCGCCGTCTTCCTGTCGGCGGCCGGCACTGTAGATTAACAGGAAAC | |
| | TTCCAAGATGGAAACTTTGTCTTTCCCCAGATATAATGTAGCTGAGATT | |
| | GTGATTCATATTCGCAATAAGATCTTAACAGGAGCTGATGGTAAAAACC | |
| | TCACCAAGAATGATCTTTATCCAAATCCAAAGCCTGAAGTCTTGCACAT | |
| | GATCTACATGAGAGCCTTACAAATAGTATATGGAATTCGACTGGAACAT | |
| | TTTTACATGATGCCAGTGAACTCTGAAGTCATGTATCCACATTTAATGG | |
| | AAGGCTTCTTACCATTCAGCAATTTAGTTACTCATCTGGACTCATTTTTG | |
| | CCTATCTGCCGGGTGAATGACTTTGAGACTGCTGATATTCTATGTCCAA | |
| | AAGCAAAACGGACAAGTCGGTTTTTAAGTGGCATTATCAACTTTATTCA | |
| | CTTCAGAGAAGCATGCCGTGAAACGTATATGGAATTTCTTTGGCAATAT | |
| | AAATCCTCTGCGGACAAAATGCAACAGTTAAACGCCGCACACCAGGAG | |
| | GCATTAATGAAACTGGAGAGACTTGATTCTGTTCCAGTTGAAGAGCAAG | |
| | AAGAGTTCAAGCAGCTTTCAGATGGAATTCAGGAGCTACAACAATCAC | |
| | TAAATCAGGATTTTCATCAAAAAACGATAGTGCTGCAAGAGGGAAATT | |
| | CCCAAAAGAAGTCAAATATTTCAGAGAAAACCAAGCGTTTGAATGAAC | |
| | TAAAATTGTCGGTGGTTTCTTTGAAAGAAATACAAGAGAGTTTGAAAAC | |
| | AAAAATTGTGGATTCTCCAGAGAAGTTAAAGAATTATAAAGAAAAAAT | |
| | GAAAGATACGGTCCAGAAGCTTAAAAATGCCAGACAAGAAGTGGTGGA | |
| | GAAATATGAAATCTATGGAGACTCAGTTGACTGCCTGCCTTCATGTCAG | |
| | TTGGAAGTGCAGTTATATCAAAAGAAAATACAGGACCTTTCAGATAAT | |
| | AGGGAAAAATTAGCCAGTATCTTAAAGGAGAGCCTGAACTTGGAGGAC | |
| | CAAATTGAGAGTGATGAGTCAGAACTGAAGAAATTGAAGACTGAAGAA | |
| | AATTCGTTCAAAAGACTGATGATTGTGAAGAAGGAAAAACTTGCCACA | |
| | GCACAATTCAAAATAAATAAGAAGCATGAAGATGTTAAGCAATACAAA | |
| | CGCACAGTAATTGAGGATTGCAATAAAGTTCAAGAAAAAAGAGGTGCT | |
| | GTCTATGAACGAGTAACCACAATTAATCAAGAAATCCAAAAAATTAAA | |
| | CTTGGAATTCAACAACTAAAAGATGCTGCTGAAAGGGAGAAACTGAAG | |
| | TCCCAGGAAATATTTCTAAACTTGAAAACTGCTTTGGAGAAATACCACG | |
| | ACGGTATTGAAAAGGCAGCAGAGGACTCCTATGCTAAGATAGATGAGA | |
| | AGACAGCTGAACTGAAGAGGAAGATGTTCAAAATGTCAACCTGATTAA | |
| | CAAAATTACATGTCTTTTTGTAAATGGCTTGCCATCTTTTAATTTTCTAT | |
| | TTAGAAAGAAAAGTTGAAGCGAATGGAAGTATCAGAAGTACCAAATAA | |
| | TGTTGGCTTCATCAGTTTTTATACACTCTCATAAGTAGTTAATAAGATGA | |
| | ATTTAATGTAGGCTTTTATTAATTTATAATTAAAATAACTTGTGCAGCTA | |
| | TTCATGTCTCTACTCTGCCCCTTGTTGTAAATAGTTTGAGTAAAACAAAA | |
| | CTAGTTACCTTTGAAATATATATATTTTTTTCTGTTACTATC | |
| BC041846 | GGCTAGCGCGGGAGGTGGAGAAAGAGGCTTGGGCGGCCCCGCTGTAGC | 159 |
| | CGCGTGTGGGAGGACGCACGGGCCTGCTTCAAAGCTTTGGGATAACAG | |
| | CGCCTCCGGGGGATAATGAATGCGGAGCCTCCGTTTTCAGTCGACTTCA | |
| | GATGTGTCTCCACTTTTTTCCGCTGTAGCCGCAAGGCAAGGAAACATTT | |
| | CTCTTCCCGTACTGAGGAGGCTGAGGAGTGCACTGGGTGTTCTTTTCTC | |
| | CTCTAACCCAGAACTGCGAGACAGAGGCTGAGTCCCTGTAAAGAACAG | |
| | CTCCAGAAAAGCCAGGAGAGCGCAGGAGGGCATCCGGGAGGCCAGGA | |
| | GGGGTTCGCTGGGGCCTCAACCGCACCCACATCGGTCCCACCTGCGAGG | |
| | GGGCGGGACCTCGTGGCGCTGGACCAATCAGCACCCACCTGCGCTCAC | |
| | CTGGCCTCCTCCCGCTGGCTCCCGGGGGCTGCGGTGCTCAAAGGGGCAA | |
| | GAGCTGAGCGGAACACCGGCCCGCCGTCGCGGCAGCTGCTTCACCCCTC | |
| | TCTCTGCAGCCATGGGGCTCCCTCGTGGACCTCTCGCGTCTCTCCTCCTT | |
| | CTCCAGGTTTGCTGGCTGCAGTGCGCGGCCTCCGAGCCGTGCCGGGCGG | |
| | TCTTCAGGGAGGCTGAAGTGACCTTGGAGGCGGGAGGCGCGGAGCAGG | |
| | AGCCCGGCCAGGCGCTGGGGAAAGTATTCATGGGCTGCCCTGGGCAAG | |
| | AGCCAGCTCTGTTTAGCACTGATAATGATGACTTCACTGTGCGGAATGG | |
| | CGAGACAGTCCAGGAAAGAAGGTCACTGAAGGAAAGGAATCCATTGAA | |
| | GATCTTCCCATCCAAACGTATCTTACGAAGACACAAGAGAGATTGGGTG | |
| | GTTGCTCCAATATCTGTCCCTGAAAATGGCAAGGGTCCCTTCCCCCAGA | |
| | GACTGAATCAGCTCAAGTCTAATAAAGATAGAGACACCAAGATTTTCTA | |
| | CAGCATCACGGGGCCGGGGGCAGACAGCCCCCCTGAGGGTGTCTTCGC | |
| | TGTAGAGAAGGAGACAGGCTGGTTGTTGTTGAATAAGCCACTGGACCG | |
| | GGAGGAGATTGCCAAGTATGAGCTCTTTGGCCACGCTGTGTCAGAGAAT | |
| | GGTGCCTCAGTGGAGGACCCCATGAACATCTCCATCATAGTGACCGACC | |
| | AGAATGACCACAAGCCCAAGTTTACCCAGGACACCTTCCGAGGGAGTG | |
| | TCTTAGAGGGAGTCCTACCAGGTACTTCTGTGATGCAGATGACAGCCAC | |
| | AGATGAGGATGATGCCATCTACACCTACAATGGGGTGGTTGCTTACTCC | |
| | ATCCATAGCCAAGAACCAAAGGACCCACACGACCTCATGTTCACAATTC | |
| | ACCGGAGCACAGGCACCATCAGCGTCATCTCCAGTGGCCTGGACCGGG | |
| | AAAAAGTCCCTGAGTACACACTGACCATCCAGGCCACAGACATGGATG | |
| | GGGACGGCTCCACCACCACGGCAGTGGCAGTAGTGGAGATCCTTGATG | |
| | CCAATGACAATGCTCCCATGTTTGACCCCCAGAAGTACGAGGCCCATGT | |
| | GCCTGAGAATGCAGTGGGCCATGAGGTGCAGAGGCTGACGGTCACTGA | |
| | TCTGGACGCCCCCAACTCACCAGCGTGGCGTGCCACCTACCTTATCATG | |
| | GGCGGTGACGACGGGGACCATTTTACCATCACCACCCACCCTGAGAGC | |
| | AACCAGGGCATCCTGACAACCAGGAAGGGTTTGGATTTTGAGGCCAAA | |
| | AACCAGCACACCCTGTACGTTGAAGTGACCAACGAGGCCCCTTTTGTGC | |
| | TGAAGCTCCCAACCTCCACAGCCACCATAGTGGTCCACGTGGAGGATGT | |
| | GAATGAGGCACCTGTGTTTGTCCCACCCTCCAAAGTCGTTGAGGTCCAG | |
| | GAGGGCATCCCCACTGGGGAGCCTGTGTGTGTCTACACTGCAGAAGAC | |
| | CCTGACAAGGAGAATCAAAAGATCAGCTACCGCATCCTGAGAGACCCA | |
| | GCAGGGTGGCTAGCCATGGACCCAGACAGTGGGCAGGTCACAGCTGTG | |
| | GGCACCCTCGACCGTGAGGATGAGCAGTTTGTGAGGAACAACATCTAT | |
| | GAAGTCATGGTCTTGGCCATGGACAATGGAAGCCCTCCCACCACTGGCA | |
| | CGGGAACCCTTCTGCTAACACTGATTGATGTCAACGACCATGGCCCAGT | |
| | CCCTGAGCCCCGTCAGATCACCATCTGCAACCAAAGCCCTGTGCGCCAG | |
| | GTGCTGAACATCACGGACAAGGACCTGTCTCCCCACACCTCCCCTTTCC | |
| | AGGCCCAGCTCACAGATGACTCAGACATCTACTGGACGGCAGAGGTCA | |
| | ACGAGGAAGGTGACACAGTGGTCTTGTCCCTGAAGAAGTTCCTGAAGC | |
| | AGGATACATATGACGTGCACCTTTCTCTGTCTGACCATGGCAACAAAGA | |
| | GCAGCTGACGGTGATCAGGGCCACTGTGTGCGACTGCCATGGCCATGTC | |
| | GAAACCTGCCCTGGACCCTGGAAAGGAGGTTTCATCCTCCCTGTGCTGG | |
| | GGGCTGTCCTGGCTCTGCTGTTCCTCCTGCTGGTGCTGCTTTTGTTGGTG | |
| | AGAAAGAAGCGGAAGATCAAGGAGCCCCTCCTACTCCCAGAAGATGAC | |
| | ACCCGTGACAACGTCTTCTACTATGGCGAAGAGGGGGGTGGCGAAGAG | |
| | GACCAGGACTATGACATCACCCAGCTCCACCGAGGTCTGGAGGCCAGG | |
| | CCGGAGGTGGTTCTCCGCAATGACGTGGCACCAACCATCATCCCGACAC | |
| | CCATGTACCGTCCTAGGCCAGCCAACCCAGATGAAATCGGCAACTTTAT | |
| | AATTGAGAACCTGAAGGCGGCTAACACAGACCCCACAGCCCCGCCCTA | |
| | CGACACCCTCTTGGTGTTCGACTATGAGGGCAGCGGCTCCGACGCCGCG | |
| | TCCCTGAGCTCCCTCACCTCCTCCGCCTCCGACCAAGACCAAGATTACG | |
| | ATTATCTGAACGAGTGGGGCAGCCGCTTCAAGAAGCTGGCAGACATGT | |
| | ACGGTGGCGGGGAGGACGACTAGGCGGCCTGCCTGCAGGGCTGGGGAC | |
| | CAAACGTCAGGCCACAGAGCATCTCCAAGGGGTCTCAGTTCCCCCTTCA | |
| | GCTGAGGACTTCGGAGCTTGTCAGGAAGTGGCCGTAGCAACTTGGCGG | |
| | AGACAGGCTATGAGTCTGACGTTAGAGTGGTTGCTTCCTTAGCCTTTCA | |
| | GGATGGAGGAATGTGGGCAGTTTGACTTCAGCACTGAAAACCTCTCCAC | |
| | CTGGGCCAGGGTTGCCTCAGAGGCCAAGTTTCCAGAAGCCTCTTACCTG | |
| | CCGTAAAATGCTCAACCCTGTGTCCTGGGCCTGGGCCTGCTGTGACTGA | |
| | CCTACAGTGGACTTTCTCTCTGGAATGGAACCTTCTTAGGCCTCCTGGTG | |
| | CAACTTAATTTTTTTTTTTAATGCTATCTTCAAAACGTTAGAGAAAGTTC | |
| | TTCAAAAGTGCAGCCCAGAGCTGCTGGGCCCACTGGCCGTCCTGCATTT | |
| | CTGGTTTCCAGACCCCAATGCCTCCCATTCGGATGGATCTCTGCGTTTTT | |
| | ATACTGAGTGTGCCTAGGTTGCCCCTTATTTTTTATTTTCCCTGTTGCGTT | |
| | GCTATAGATGAAGGGTGAGGACAATCGTGTATATGTACTAGAACTTTTT | |
| | TATTAAAGAAACTTTTCCCAAAAAAAAAAAAAAAA | |
| NM_016343 | GAGACCAGAAGCGGGCGAATTGGGCACCGGTGGCGGCTGCGGGCAGTT | 160 |
| | TGAATTAGACTCTGGGCTCCAGCCCGCCGAAGCCGCGCCAGAACTGTAC | |
| | TCTCCGAGAGGTCGTTTTCCCGTCCCCGAGAGCAAGTTTATTTACAAAT | |
| | GTTGGAGTAATAAAGAAGGCAGAACAAAATGAGCTGGGCTTTGGAAGA | |
| | ATGGAAAGAAGGGCTGCCTACAAGAGCTCTTCAGAAAATTCAAGAGCT | |
| | TGAAGGACAGCTTGACAAACTGAAGAAGGAAAAGCAGCAAAGGCAGT | |
| | TTCAGCTTGACAGTCTCGAGGCTGCGCTGCAGAAGCAAAAACAGAAGG | |
| | TTGAAAATGAAAAAACCGAGGGTACAAACCTGAAAAGGGAGAATCAA | |
| | AGATTGATGGAAATATGTGAAAGTCTGGAGAAAACTAAGCAGAAGATT | |
| | TCTCATGAACTTCAAGTCAAGGAGTCACAAGTGAATTTCCAGGAAGGA | |
| | CAACTGAATTCAGGCAAAAAACAAATAGAAAAACTGGAACAGGAACTT | |
| | AAAAGGTGTAAATCTGAGCTTGAAAGAAGCCAACAAGCTGCGCAGTCT | |
| | GCAGATGTCTCTCTGAATCCATGCAATACACCACAAAAAATTTTTACAA | |
| | CTCCACTAACACCAAGTCAATATTATAGTGGTTCCAAGTATGAAGATCT | |
| | AAAAGAAAAATATAATAAAGAGGTTGAAGAACGAAAAAGATTAGAGG | |
| | CAGAGGTTAAAGCCTTGCAGGCTAAAAAAGCAAGCCAGACTCTTCCAC | |
| | AAGCCACCATGAATCACCGCGACATTGCCCGGCATCAGGCTTCATCATC | |
| | TGTGTTCTCATGGCAGCAAGAGAAGACCCCAAGTCATCTTTCATCTAAT | |
| | TCTCAAAGAACTCCAATTAGGAGAGATTTCTCTGCATCTTACTTTTCTGG | |
| | GGAACAAGAGGTGACTCCAAGTCGATCAACTTTGCAAATAGGGAAAAG | |
| | AGATGCTAATAGCAGTTTCTTTGACAATTCTAGCAGTCCTCATCTTTTGG | |
| | ATCAATTAAAAGCGCAGAATCAAGAGCTAAGAAACAAGATTAATGAGT | |
| | TGGAACTACGCCTGCAAGGACATGAAAAAGAAATGAAAGGCCAAGTGA | |
| | ATAAGTTTCAAGAACTCCAACTCCAACTGGAGAAAGCAAAAGTGGAAT | |
| | TAATTGAAAAAGAGAAAGTTTTGAACAAATGTAGGGATGAACTAGTGA | |
| | GAACAACAGCACAATACGACCAGGCGTCAACCAAGTATACTGCATTGG | |
| | AACAAAAACTGAAAAAATTGACGGAAGATTTGAGTTGTCAGCGACAAA | |
| | ATGCAGAAAGTGCCAGATGTTCTCTGGAACAGAAAATTAAGGAAAAAG | |
| | AAAAGGAGTTTCAAGAGGAGCTCTCCCGTCAACAGCGTTCTTTCCAAAC | |
| | ACTGGACCAGGAGTGCATCCAGATGAAGGCCAGACTCACCCAGGAGTT | |
| | ACAGCAAGCCAAGAATATGCACAACGTCCTGCAGGCTGAACTGGATAA | |
| | ACTCACATCAGTAAAGCAACAGCTAGAAAACAATTTGGAAGAGTTTAA | |
| | GCAAAAGTTGTGCAGAGCTGAACAGGCGTTCCAGGCGAGTCAGATCAA | |
| | GGAGAATGAGCTGAGGAGAAGCATGGAGGAAATGAAGAAGGAAAACA | |
| | ACCTCCTTAAGAGTCACTCTGAGCAAAAGGCCAGAGAAGTCTGCCACCT | |
| | GGAGGCAGAACTCAAGAACATCAAACAGTGTTTAAATCAGAGCCAGAA | |
| | TTTTGCAGAAGAAATGAAAGCGAAGAATACCTCTCAGGAAACCATGTT | |
| | AAGAGATCTTCAAGAAAAAATAAATCAGCAAGAAAACTCCTTGACTTT | |
| | AGAAAAACTGAAGCTTGCTGTGGCTGATCTGGAAAAGCAGCGAGATTG | |
| | TTCTCAAGACCTTTTGAAGAAAAGAGAACATCACATTGAACAACTTAAT | |
| | GATAAGTTAAGCAAGACAGAGAAAGAGTCCAAAGCCTTGCTGAGTGCT | |
| | TTAGAGTTAAAAAAGAAAGAATATGAAGAATTGAAAGAAGAGAAAAC | |
| | TCTGTTTTCTTGTTGGAAAAGTGAAAACGAAAAACTTTTAACTCAGATG | |
| | GAATCAGAAAAGGAAAACTTGCAGAGTAAAATTAATCACTTGGAAACT | |
| | TGTCTGAAGACACAGCAAATAAAAAGTCATGAATACAACGAGAGAGTA | |
| | AGAACGCTGGAGATGGACAGAGAAAACCTAAGTGTCGAGATCAGAAAC | |
| | CTTCACAACGTGTTAGACAGTAAGTCAGTGGAGGTAGAGACCCAGAAA | |
| | CTAGCTTATATGGAGCTACAGCAGAAAGCTGAGTTCTCAGATCAGAAA | |
| | CATCAGAAGGAAATAGAAAATATGTGTTTGAAGACTTCTCAGCTTACTG | |
| | GGCAAGTTGAAGATCTAGAACACAAGCTTCAGTTACTGTCAAATGAAA | |
| | TAATGGACAAAGACCGGTGTTACCAAGACTTGCATGCCGAATATGAGA | |
| | GCCTCAGGGATCTGCTAAAATCCAAAGATGCTTCTCTGGTGACAAATGA | |
| | AGATCATCAGAGAAGTCTTTTGGCTTTTGATCAGCAGCCTGCCATGCAT | |
| | CATTCCTTTGCAAATATAATTGGAGAACAAGGAAGCATGCCTTCAGAGA | |
| | GGAGTGAATGTCGTTTAGAAGCAGACCAAAGTCCGAAAAATTCTGCCA | |
| | TCCTACAAAATAGAGTTGATTCACTTGAATTTTCATTAGAGTCTCAAAA | |
| | ACAGATGAACTCAGACCTGCAAAAGCAGTGTGAAGAGTTGGTGCAAAT | |
| | CAAAGGAGAAATAGAAGAAAATCTCATGAAAGCAGAACAGATGCATC | |
| | AAAGTTTTGTGGCTGAAACAAGTCAGCGCATTAGTAAGTTACAGGAAG | |
| | ACACTTCTGCTCACCAGAATGTTGTTGCTGAAACCTTAAGTGCCCTTGA | |
| | GAACAAGGAAAAAGAGCTGCAACTTTTAAATGATAAGGTAGAAACTGA | |
| | GCAGGCAGAGATTCAAGAATTAAAAAAGAGCAACCATCTACTTGAAGA | |
| | CTCTCTAAAGGAGCTACAACTTTTATCCGAAACCCTAAGCTTGGAGAAG | |
| | AAAGAAATGAGTTCCATCATTTCTCTAAATAAAAGGGAAATTGAAGAG | |
| | CTGACCCAAGAGAATGGGACTCTTAAGGAAATTAATGCATCCTTAAATC | |
| | AAGAGAAGATGAACTTAATCCAGAAAAGTGAGAGTTTTGCAAACTATA | |
| | TAGATGAAAGGGAGAAAAGCATTTCAGAGTTATCTGATCAGTACAAGC | |
| | AAGAAAAACTTATTTTACTACAAAGATGTGAAGAAACCGGAAATGCAT | |
| | ATGAGGATCTTAGTCAAAAATACAAAGCAGCACAGGAAAAGAATTCTA | |
| | AATTAGAATGCTTGCTAAATGAATGCACTAGTCTTTGTGAAAATAGGAA | |
| | AAATGAGTTGGAACAGCTAAAGGAAGCATTTGCAAAGGAACACCAAGA | |
| | ATTCTTAACAAAATTAGCATTTGCTGAAGAAAGAAATCAGAATCTGATG | |
| | CTAGAGTTGGAGACAGTGCAGCAAGCTCTGAGATCTGAGATGACAGAT | |
| | AACCAAAACAATTCTAAGAGCGAGGCTGGTGGTTTAAAGCAAGAAATC | |
| | ATGACTTTAAAGGAAGAACAAAACAAAATGCAAAAGGAAGTTAATGAC | |
| | TTATTACAAGAGAATGAACAGCTGATGAAGGTAATGAAGACTAAACAT | |
| | GAATGTCAAAATCTAGAATCAGAACCAATTAGGAACTCTGTGAAAGAA | |
| | AGAGAGAGTGAGAGAAATCAATGTAATTTTAAACCTCAGATGGATCTT | |
| | GAAGTTAAAGAAATTTCTCTAGATAGTTATAATGCGCAGTTGGTGCAAT | |
| | TAGAAGCTATGCTAAGAAATAAGGAATTAAAACTTCAGGAAAGTGAGA | |
| | AGGAGAAGGAGTGCCTGCAGCATGAATTACAGACAATTAGAGGAGATC | |
| | TTGAAACCAGCAATTTGCAAGACATGCAGTCACAAGAAATTAGTGGCC | |
| | TTAAAGACTGTGAAATAGATGCGGAAGAAAAGTATATTTCAGGGCCTC | |
| | ATGAGTTGTCAACAAGTCAAAACGACAATGCACACCTTCAGTGCTCTCT | |
| | GCAAACAACAATGAACAAGCTGAATGAGCTAGAGAAAATATGTGAAAT | |
| | ACTGCAGGCTGAAAAGTATGAACTCGTAACTGAGCTGAATGATTCAAG | |
| | GTCAGAATGTATCACAGCAACTAGGAAAATGGCAGAAGAGGTAGGGAA | |
| | ACTACTAAATGAAGTTAAAATATTAAATGATGACAGTGGTCTTCTCCAT | |
| | GGTGAGTTAGTGGAAGACATACCAGGAGGTGAATTTGGTGAACAACCA | |
| | AATGAACAGCACCCTGTGTCTTTGGCTCCATTGGACGAGAGTAATTCCT | |
| | ACGAGCACTTGACATTGTCAGACAAAGAAGTTCAAATGCACTTTGCCGA | |
| | ATTGCAAGAGAAATTCTTATCTTTACAAAGTGAACACAAAATTTTACAT | |
| | GATCAGCACTGTCAGATGAGCTCTAAAATGTCAGAGCTGCAGACCTATG | |
| | TTGACTCATTAAAGGCCGAAAATTTGGTCTTGTCAACGAATCTGAGAAA | |
| | CTTTCAAGGTGACTTGGTGAAGGAGATGCAGCTGGGCTTGGAGGAGGG | |
| | GCTCGTTCCATCCCTGTCATCCTCTTGTGTGCCTGACAGCTCTAGTCTTA | |
| | GCAGTTTGGGAGACTCCTCCTTTTACAGAGCTCTTTTAGAACAGACAGG | |
| | AGATATGTCTCTTTTGAGTAATTTAGAAGGGGCTGTTTCAGCAAACCAG | |
| | TGCAGTGTAGATGAAGTATTTTGCAGCAGTCTGCAGGAGGAGAATCTG | |
| | ACCAGGAAAGAAACCCCTTCGGCCCCAGCGAAGGGTGTTGAAGAGCTT | |
| | GAGTCCCTCTGTGAGGTGTACCGGCAGTCCCTCGAGAAGCTAGAAGAG | |
| | AAAATGGAAAGTCAAGGGATTATGAAAAATAAGGAAATTCAAGAGCTC | |
| | GAGCAGTTATTAAGTTCTGAAAGGCAAGAGCTTGACTGCCTTAGGAAG | |
| | CAGTATTTGTCAGAAAATGAACAGTGGCAACAGAAGCTGACAAGCGTG | |
| | ACTCTGGAGATGGAGTCCAAGTTGGCGGCAGAAAAGAAACAGACGGAA | |
| | CAACTGTCACTTGAGCTGGAAGTAGCACGACTCCAGCTACAAGGTCTGG | |
| | ACTTAAGTTCTCGGTCTTTGCTTGGCATCGACACAGAAGATGCTATTCA | |
| | AGGCCGAAATGAGAGCTGTGACATATCAAAAGAACATACTTCAGAAAC | |
| | TACAGAAAGAACACCAAAGCATGATGTTCATCAGATTTGTGATAAAGA | |
| | TGCTCAGCAGGACCTCAATCTAGACATTGAGAAAATAACTGAGACTGG | |
| | TGCAGTGAAACCCACAGGAGAGTGCTCTGGGGAACAGTCCCCAGATAC | |
| | CAATTATGAGCCTCCAGGGGAAGATAAAACCCAGGGCTCTTCAGAATG | |
| | CATTTCTGAATTGTCATTTTCTGGTCCTAATGCTTTGGTACCTATGGATT | |
| | TCCTGGGGAATCAGGAAGATATCCATAATCTTCAACTGCGGGTAAAAG | |
| | AGACATCAAATGAGAATTTGAGATTACTTCATGTGATAGAGGACCGTG | |
| | ACAGAAAAGTTGAAAGTTTGCTAAATGAAATGAAAGAATTAGACTCAA | |
| | AACTCCATTTACAGGAGGTACAACTAATGACCAAAATTGAAGCATGCA | |
| | TAGAATTGGAAAAAATAGTTGGGGAACTTAAGAAAGAAAACTCAGATT | |
| | TAAGTGAAAAATTGGAATATTTTTCTTGTGATCACCAGGAGTTACTCCA | |
| | GAGAGTAGAAACTTCTGAAGGCCTCAATTCTGATTTAGAAATGCATGCA | |
| | GATAAATCATCACGTGAAGATATTGGAGATAATGTGGCCAAGGTGAAT | |
| | GACAGCTGGAAGGAGAGATTTCTTGATGTGGAAAATGAGCTGAGTAGG | |
| | ATCAGATCGGAGAAAGCTAGCATTGAGCATGAAGCCCTCTACCTGGAG | |
| | GCTGACTTAGAGGTAGTTCAAACAGAGAAGCTATGTTTAGAAAAAGAC | |
| | AATGAAAATAAGCAGAAGGTTATTGTCTGCCTTGAAGAAGAACTCTCA | |
| | GTGGTCACAAGTGAGAGAAACCAGCTTCGTGGAGAATTAGATACTATG | |
| | TCAAAAAAAACCACGGCACTGGATCAGTTGTCTGAAAAAATGAAGGAG | |
| | AAAACACAAGAGCTTGAGTCTCATCAAAGTGAGTGTCTCCATTGCATTC | |
| | AGGTGGCAGAGGCAGAGGTGAAGGAAAAGACGGAACTCCTTCAGACTT | |
| | TGTCCTCTGATGTGAGTGAGCTGTTAAAAGACAAAACTCATCTCCAGGA | |
| | AAAGCTGCAGAGTTTGGAAAAGGACTCACAGGCACTGTCTTTGACAAA | |
| | ATGTGAGCTGGAAAACCAAATTGCACAACTGAATAAAGAGAAAGAATT | |
| | GCTTGTCAAGGAATCTGAAAGCCTGCAGGCCAGACTGAGTGAATCAGA | |
| | TTATGAAAAGCTGAATGTCTCCAAGGCCTTGGAGGCCGCACTGGTGGA | |
| | GAAAGGTGAGTTCGCATTGAGGCTGAGCTCAACACAGGAGGAAGTGCA | |
| | TCAGCTGAGAAGAGGCATCGAGAAACTGAGAGTTCGCATTGAGGCCGA | |
| | TGAAAAGAAGCAGCTGCACATCGCAGAGAAACTGAAAGAACGCGAGC | |
| | GGGAGAATGATTCACTTAAGGATAAAGTTGAGAACCTTGAAAGGGAAT | |
| | TGCAGATGTCAGAAGAAAACCAGGAGCTAGTGATTCTTGATGCCGAGA | |
| | ATTCCAAAGCAGAAGTAGAGACTCTAAAAACACAAATAGAAGAGATGG | |
| | CCAGAAGCCTGAAAGTTTTTGAATTAGACCTTGTCACGTTAAGGTCTGA | |
| | AAAAGAAAATCTGACAAAACAAATACAAGAAAAACAAGGTCAGTTGTC | |
| | AGAACTAGACAAGTTACTCTCTTCATTTAAAAGTCTGTTAGAAGAAAAG | |
| | GAGCAAGCAGAGATACAGATCAAAGAAGAATCTAAAACTGCAGTGGA | |
| | GATGCTTCAGAATCAGTTAAAGGAGCTAAATGAGGCAGTAGCAGCCTT | |
| | GTGTGGTGACCAAGAAATTATGAAGGCCACAGAACAGAGTCTAGACCC | |
| | ACCAATAGAGGAAGAGCATCAGCTGAGAAATAGCATTGAAAAGCTGAG | |
| | AGCCCGCCTAGAAGCTGATGAAAAGAAGCAGCTCTGTGTCTTACAACA | |
| | ACTGAAGGAAAGTGAGCATCATGCAGATTTACTTAAGGGTAGAGTGGA | |
| | GAACCTTGAAAGAGAGCTAGAGATAGCCAGGACAAACCAAGAGCATGC | |
| | AGCTCTTGAGGCAGAGAATTCCAAAGGAGAGGTAGAGACCCTAAAAGC | |
| | AAAAATAGAAGGGATGACCCAAAGTCTGAGAGGTCTGGAATTAGATGT | |
| | TGTTACTATAAGGTCAGAAAAAGAAAATCTGACAAATGAATTACAAAA | |
| | AGAGCAAGAGCGAATATCTGAATTAGAAATAATAAATTCATCATTTGA | |
| | AAATATTTTGCAAGAAAAAGAGCAAGAGAAAGTACAGATGAAAGAAA | |
| | AATCAAGCACTGCCATGGAGATGCTTCAAACACAATTAAAAGAGCTCA | |
| | ATGAGAGAGTGGCAGCCCTGCATAATGACCAAGAAGCCTGTAAGGCCA | |
| | AAGAGCAGAATCTTAGTAGTCAAGTAGAGTGTCTTGAACTTGAGAAGG | |
| | CTCAGTTGCTACAAGGCCTTGATGAGGCCAAAAATAATTATATTGTTTT | |
| | GCAATCTTCAGTGAATGGCCTCATTCAAGAAGTAGAAGATGGCAAGCA | |
| | GAAACTGGAGAAGAAGGATGAAGAAATCAGTAGACTGAAAAATCAAA | |
| | TTCAAGACCAAGAGCAGCTTGTCTCTAAACTGTCCCAGGTGGAAGGAG | |
| | AGCACCAACTTTGGAAGGAGCAAAACTTAGAACTGAGAAATCTGACAG | |
| | TGGAATTGGAGCAGAAGATCCAAGTGCTACAATCCAAAAATGCCTCTTT | |
| | GCAGGACACATTAGAAGTGCTGCAGAGTTCTTACAAGAATCTAGAGAA | |
| | TGAGCTTGAATTGACAAAAATGGACAAAATGTCCTTTGTTGAAAAAGTA | |
| | AACAAAATGACTGCAAAGGAAACTGAGCTGCAGAGGGAAATGCATGA | |
| | GATGGCACAGAAAACAGCAGAGCTGCAAGAAGAACTCAGTGGAGAGA | |
| | AAAATAGGCTAGCTGGAGAGTTGCAGTTACTGTTGGAAGAAATAAAGA | |
| | GCAGCAAAGATCAATTGAAGGAGCTCACACTAGAAAATAGTGAATTGA | |
| | AGAAGAGCCTAGATTGCATGCACAAAGACCAGGTGGAAAAGGAAGGG | |
| | AAAGTGAGAGAGGAAATAGCTGAATATCAGCTACGGCTTCATGAAGCT | |
| | GAAAAGAAACACCAGGCTTTGCTTTTGGACACAAACAAACAGTATGAA | |
| | GTAGAAATCCAGACATACCGAGAGAAATTGACTTCTAAAGAAGAATGT | |
| | CTCAGTTCACAGAAGCTGGAGATAGACCTTTTAAAGTCTAGTAAAGAA | |
| | GAGCTCAATAATTCATTGAAAGCTACTACTCAGATTTTGGAAGAATTGA | |
| | AGAAAACCAAGATGGACAATCTAAAATATGTAAATCAGTTGAAGAAGG | |
| | AAAATGAACGTGCCCAGGGGAAAATGAAGTTGTTGATCAAATCCTGTA | |
| | AACAGCTGGAAGAGGAAAAGGAGATACTGCAGAAAGAACTCTCTCAAC | |
| | TTCAAGCTGCACAGGAGAAGCAGAAAACAGGTACTGTTATGGATACCA | |
| | AGGTCGATGAATTAACAACTGAGATCAAAGAACTGAAAGAAACTCTTG | |
| | AAGAAAAAACCAAGGAGGCAGATGAATACTTGGATAAGTACTGTTCCT | |
| | TGCTTATAAGCCATGAAAAGTTAGAGAAAGCTAAAGAGATGTTAGAGA | |
| | CACAAGTGGCCCATCTGTGTTCACAGCAATCTAAACAAGATTCCCGAGG | |
| | GTCTCCTTTGCTAGGTCCAGTTGTTCCAGGACCATCTCCAATCCCTTCTG | |
| | TTACTGAAAAGAGGTTATCATCTGGCCAAAATAAAGCTTCAGGCAAGA | |
| | GGCAAAGATCCAGTGGAATATGGGAGAATGGTAGAGGACCAACACCTG | |
| | CTACCCCAGAGAGCTTTTCTAAAAAAAGCAAGAAAGCAGTCATGAGTG | |
| | GTATTCACCCTGCAGAAGACACGGAAGGTACTGAGTTTGAGCCAGAGG | |
| | GACTTCCAGAAGTTGTAAAGAAAGGGTTTGCTGACATCCCGACAGGAA | |
| | AGACTAGCCCATATATCCTGCGAAGAACAACCATGGCAACTCGGACCA | |
| | GCCCCCGCCTGGCTGCACAGAAGTTAGCGCTATCCCCACTGAGTCTCGG | |
| | CAAAGAAAATCTTGCAGAGTCCTCCAAACCAACAGCTGGTGGCAGCAG | |
| | ATCACAAAAGGTCAAAGTTGCTCAGCGGAGCCCAGTAGATTCAGGCAC | |
| | CATCCTCCGAGAACCCACCACGAAATCCGTCCCAGTCAATAATCTTCCT | |
| | GAGAGAAGTCCGACTGACAGCCCCAGAGAGGGCCTGAGGGTCAAGCGA | |
| | GGCCGACTTGTCCCCAGCCCCAAAGCTGGACTGGAGTCCAACGGCAGT | |
| | GAGAACTGTAAGGTCCAGTGAAGGCACTTTGTGTGTCAGTACCCCTGGG | |
| | AGGTGCCAGTCATTGAATAGATAAGGCTGTGCCTACAGGACTTCTCTTT | |
| | AGTCAGGGCATGCTTTATTAGTGAGGAGAAAACAATTCCTTAGAAGTCT | |
| | TAAATATATTGTACTCTTTAGATCTCCCATGTGTAGGTATTGAAAAAGTT | |
| | TGGAAGCACTGATCACCTGTTAGCATTGCCATTCCTCTACTGCAATGTA | |
| | AATAGTATAAAGCTATGTATATAAAGCTTTTTGGTAATATGTTACAATT | |
| | AAAATGACAAGCACTATATCACAATCTCTGTTTGTATGTGGGTTTTACA | |
| | CTAAAAAAATGCAAAACACATTTTATTCTTCTAATTAACAGCTCCTAGG | |
| | AAAATGTAGACTTTTGCTTTATGATATTCTATCTGTAGTATGAGGCATG | |
| | GAATAGTTTTGTATCGGGAATTTCTCAGAGCTGAGTAAAATGAAGGAA | |
| | AAGCATGTTATGTGTTTTTAAGGAAAATGTGCACACATATACATGTAGG | |
| | AGTGTTTATCTTTCTCTTACAATCTGTTTTAGACATCTTTGCTTATGAAA | |
| | CCTGTACATATGTGTGTGTGGGTATGTGTTTATTTCCAGTGAGGGCTGC | |
| | AGGCTTCCTAGAGGTGTGCTATACCATGCGTCTGTCGTTGTGCTTTTTTC | |
| | TGTTTTTAGACCAATTTTTTACAGTTCTTTGGTAAGCATTGTCGTATCTG | |
| | GTGATGGATTAACATATAGCCTTTGTTTTCTAATAAAATAGTCGCCTTCG | |
| | TTTTCTGTAAAAAAAAAAAAAAAAAAAAAA | |
| AB091343 | GGCACGAGGGGCCGACGCGAGCGCCGCGCTTCGCTTCAGCTGCTAGCT | 161 |
| | GGCCCAAGGGAGGCGACCGCGGAGGGTGGCGAGGGGCGGCCAGGACC | |
| | CGCAGCCCCGGGGCCGGGCCGGTCCGGACCGCCAGGGAGGGCAGGTCA | |
| | GTGGGCAGATCGCGTCCGCGGGATTCAATCTCTGCCCGCTCTGATAACA | |
| | GTCCTTTTCCCTGGCGCTCACTTCGTGCCTGGCACCCGGCTGGGCGCCTC | |
| | AAGACCGTTGTCTCTTCGATCGCTTCTTTGGACTTGGCGACCATTTCAGA | |
| | GATGTCTTCCAGAAGTACCAAAGATTTAATTAAAAGTAAGTGGGGATC | |
| | GAAGCCTAGTAACTCCAAATCCGAAACTACATTAGAAAAATTAAAGGG | |
| | AGAAATTGCACACTTAAAGACATCAGTGGATGAAATCACAAGTGGGAA | |
| | AGGAAAGCTGACTGATAAAGAGAGACACAGACTTTTGGAGAAAATTCG | |
| | AGTCCTTGAGGCTGAGAAGGAGAAGAATGCTTATCAACTCACAGAGAA | |
| | GGACAAAGAAATACAGCGACTGAGAGACCAACTGAAGGCCAGATATA | |
| | GTACTACCGCATTGCTTGAACAGCTGGAAGAGACAACGAGAGAAGGAG | |
| | AAAGGAGGGAGCAGGTGTTGAAAGCCTTATCTGAAGAGAAAGACGTAT | |
| | TGAAACAACAGTTGTCTGCTGCAACCTCACGAATTGCTGAACTTGAAAG | |
| | CAAAACCAATACACTCCGTTTATCACAGACTGTGGCTCCAAACTGCTTC | |
| | AACTCATCAATAAATAATATTCATGAAATGGAAATACAGCTGAAAGAT | |
| | GCTCTGGAGAAAAATCAGCAGTGGCTCGTGTATGATCAGCAGCGGGAA | |
| | GTCTATGTAAAAGGACTTTTAGCAAAGATCTTTGAGTTGGAAAAGAAA | |
| | ACGGAAACAGCTGCTCATTCACTCCCACAGCAGACAAAAAAGCCTGAA | |
| | TCAGAAGGTTATCTTCAAGAAGAGAAGCAGAAATGTTACAACGATCTC | |
| | TTGGCAAGTGCAAAAAAAGATCTTGAGGTTGAACGACAAACCATAACT | |
| | CAGCTGAGTTTTGAACTGAGTGAATTTCGAAGAAAATATGAAGAAACC | |
| | CAAAAAGAAGTTCACAATTTAAATCAGCTGTTGTATTCACAAAGAAGG | |
| | GCAGATGTGCAACATCTGGAAGATGATAGGCATAAAACAGAGAAGATA | |
| | CAAAAACTCAGGGAAGAGAATGATATTGCTAGGGGAAAACTTGAAGAA | |
| | GAGAAGAAGAGATCCGAAGAGCTCTTATCTCAGGTCCAGTTTCTTTACA | |
| | CATCTCTGCTAAAGCAGCAAGAAGAACAAACAAGGGTAGCTCTGTTGG | |
| | AACAACAGATGCAGGCATGTACTTTAGACTTTGAAAATGAAAAACTCG | |
| | ACCGTCAACATGTGCAGCATCAATTGCATGTAATTCTTAAGGAGCTCCG | |
| | AAAAGCAAGAAATCAAATAACACAGTTGGAATCCTTGAAACAGCTTCA | |
| | TGAGTTTGCCATCACAGAGCCATTAGTCACTTTCCAAGGAGAGACTGAA | |
| | AACAGAGAAAAAGTTGCCGCCTCACCAAAAAGTCCCACTGCTGCACTC | |
| | AATGAAAGCCTGGTGGAATGTCCCAAGTGCAATATACAGTATCCAGCC | |
| | ACTGAGCATCGCGATCTGCTTGTCCATGTGGAATACTGTTCAAAGTAGC | |
| | AAAATAAGTATTTGTTTTGATATTAAAAGATTCAATACTGTATTTTCTGT | |
| | TAGCTTGTGGGCATTTTGAATTATATATTTCACATTTTGCATAAAACTGC | |
| | CTATCTACCTTTGACACTCCAGCATGCTAGTGAATCATGTATCTTTTAGG | |
| | CTGCTGTGCATTTCTCTTGGCAGTGATACCTCCCTGACATGGTTCATCAT | |
| | CAGGCTGCAATGACAGAATGTGGTGAGCAGCGTCTACTGAGACTACTA | |
| | ACATTTTGCACTGTCAAAATACTTGGTGAGGAAAAGATAGCTCAGGTTA | |
| | TTGCTAATGGGTTAATGCACCAGCAAGCAAAATATTTTATGTTTTGGGG | |
| | GTTTGAAAAATCAAAGATAATTAACCAAGGATCTTAACTGTGTTCGCAT | |
| | TTTTTATCCAAGCACTTAGAAAACCTACAATCCTAATTTTGATGTCCATT | |
| | GTTAAGAGGTGGTGATAGATACTATTTTTTTTTTCATATTGTATAGCGGT | |
| | TATTAGAAAAGTTGGGGATTTTCTTGATCTTTATTGCTGCTTACCATTGA | |
| | AACTTAACCCAGCTGTGTTCCCCAACTCTGTTCTGCGCACGAAACAGTA | |
| | TCTGTTTGAGGCATAATCTTAAGTGGCCACACACAATGTTTTCTCTTATG | |
| | TTATCTGGCAGTAACTGTAACTTGAATTACATTAGCACATTCTGCTTAGC | |
| | TAAAATTGTTAAAATAAACTTTAATAAACCCATGTAGCCCTCTCATTTG | |
| | ATTGACAGTATTTTAGTTATTTTTGGCATTCTTAAAGCTGGGCAATGTAA | |
| | TGATCAGATCTTTGTTTGTCTGAACAGGTATTTTTATACATGCTTTTTGT | |
| | AAACCAAAAACTTTTAAATTTCTTCAGGTTTTCTAACATGCTTACCACTG | |
| | GGCTACTGTAAATGAGAAAAGAATAAAATTATTTAATGTTTTAAAAAA | |
| | AAAAAAAAA | |
| BC006428 | GGCGGCTGAGCCTGAGCGGGGATGTAGAGGCGGCGGCAGCAGAGGCG | 162 |
| | GCACTGGCGGCAAGAGCAGACGCCCGAGCCGAGCGAGAAGAGCGGCA | |
| | GAGCCTTATCCCCTGAAGCCGGGCCCCGCGTCCCAGCCCTGCCCAGCCC | |
| | GCGCCCAGCCATGCGCGCCGCCTGCTGAGTCCGGGCGCCGCACGCTGA | |
| | GCCCTCCGCCCGCGAGCCGCGCTCAGCTCGGGGGTGATTAGTTGCTTTT | |
| | TGTTGTTTTTTAATTTGGGCCGCGGGGAGGGGGAGGAGGGGCAGGTGCT | |
| | GCAGGCTCCCCCCCCTCCCCGCCTCGGGCCAGCCGCGGCGGCGCGACTC | |
| | GGGCTCCGGACCCGGGCACTGCTGGCGGCTGGAGCGGAGCGCACCGCG | |
| | GCGGTGGTGCCCAGAGCGGAGCGCAGCTCCCTGCCCCGCCCCTCCCCCT | |
| | CGGCCTCGCGGCGACGGCGGCGGTGGCGGCTTGGACGACTCGGAGAGC | |
| | CGAGTGAAGACATTTCCACCTGGACACCTGACCATGTGCCTGCCCTGAG | |
| | CAGCGAGGCCCACCAGGCATCTCTGTTGTGGGCAGCAGGGCCAGGTCC | |
| | TGGTCTGTGGACCCTCGGCAGTTGGCAGGCTCCCTCTGCAGTGGGGTCT | |
| | GGGCCTCGGCCCCACCATGTCGAGCCTCGGCGGTGGCTCCCAGGATGCC | |
| | GGCGGCAGTAGCAGCAGCAGCACCAATGGCAGCGGTGGCAGTGGCAGC | |
| | AGTGGCCCAAAGGCAGGAGCAGCAGACAAGAGTGCAGTGGTGGCTGCC | |
| | GCCGCACCAGCCTCAGTGGCAGATGACACACCACCCCCCGAGCGTCGG | |
| | AACAAGAGCGGTATCATCAGTGAGCCCCTCAACAAGAGCCTGCGCCGC | |
| | TCCCGCCCGCTCTCCCACTACTCTTCTTTTGGCAGCAGTGGTGGTAGTGG | |
| | CGGTGGCAGCATGATGGGCGGAGAGTCTGCTGACAAGGCCACTGCGGC | |
| | TGCAGCCGCTGCCTCCCTGTTGGCCAATGGGCATGACCTGGCGGCGGCC | |
| | ATGGCGGTGGACAAAAGCAACCCTACCTCAAAGCACAAAAGTGGTGCT | |
| | GTGGCCAGCCTGCTGAGCAAGGCAGAGCGGGCCACGGAGCTGGCAGCC | |
| | GAGGGACAGCTGACGCTGCAGCAGTTTGCGCAGTCCACAGAGATGCTG | |
| | AAGCGCGTGGTGCAGGAGCATCTCCCGCTGATGAGCGAGGCGGGTGCT | |
| | GGCCTGCCTGACATGGAGGCTGTGGCAGGTGCCGAAGCCCTCAATGGC | |
| | CAGTCCGACTTCCCCTACCTGGGCGCTTTCCCCATCAACCCAGGCCTCTT | |
| | CATTATGACCCCGGCAGGTGTGTTCCTGGCCGAGAGCGCGCTGCACATG | |
| | GCGGGCCTGGCTGAGTACCCCATGCAGGGAGAGCTGGCCTCTGCCATC | |
| | AGCTCCGGCAAGAAGAAGCGGAAACGCTGCGGCATGTGCGCGCCCTGC | |
| | CGGCGGCGCATCAACTGCGAGCAGTGCAGCAGTTGTAGGAATCGAAAG | |
| | ACTGGCCATCAGATTTGCAAATTCAGAAAATGTGAGGAACTCAAAAAG | |
| | AAGCCTTCCGCTGCTCTGGAGAAGGTGATGCTTCCGACGGGAGCCGCCT | |
| | TCCGGTGGTTTCAGTGACGGCGGCGGAACCCAAAGCTGCCCTCTCCGTG | |
| | CAATGTCACTGCTCGTGTGGTCTCCAGCAAGGGATTCGGGCGAAGACA | |
| | AACGGATGCACCCGTCTTTAGAACCAAAAATATTCTCTCACAGATTTCA | |
| | TTCCTGTTTTTATATATATATTTTTTGTTGTCGTTTTAACATCTCCACGTC | |
| | CCTAGCATAAAAAGAAAAAGAAAAAAATTTAAACTGCTTTTTCGGAAG | |
| | AACAACAACAAAAAAGAGGTAAAGACGAATCTATAAAGTACCGAGACT | |
| | TCCTGGGCAAAGAATGGACAATCAGTTTCCTTCCTGTGTCGATGTCGAT | |
| | GTTGTCTGTGCAGGAGATGCAGTTTTTGTGTAGAGAATGTAAATTTTCT | |
| | GTAACCTTTTGAAATCTAGTTACTAATAAGCACTACTGTAATTTAGCAC | |
| | AGTTTAACTCCACCCTCATTTAAACTTCCTTTGATTCTTTCCGACCATGA | |
| | AATAGTGCATAGTTTGCCTGGAGAATCCACTCACGTTCATAAAGAGAAT | |
| | GTTGATGGCGCCGTGTAGAAGCCGCTCTGTATCCATCCACGCGTGCAGA | |
| | GCTGCCAGCAGGGAGCTCACAGAAGGGGAGGGAGCACCAGGCCAGCT | |
| | GAGCTGCACCCACAGTCCCGAGACTGGGATCCCCCACCCCAACAGTGA | |
| | TTTTGGAAAAAAAAATGAAAGTTCTGTTCGTTTATCCATTGCGATCTGG | |
| | GGAGCCCCATCTCGATATTTCCAATCCTGGCTACTTTTCTTAGAGAAAA | |
| | TAAGTCCTTTTTTTCTGGCCTTGCTAATGGCAACAGAAGAAAGGGCTTC | |
| | TTTGCGTGGTCCCCTGCTGGTGGGGGTGGGTCCCCAGGGGGCCCCCTGC | |
| | GGCCTGGGCCCCCCTGCCCACGGCCAGCTTCCTGCTGATGAACATGCTG | |
| | TTTGTATTGTTTTAGGAAACCAGGCTGTTTTGTGAATAAAACGAATGCA | |
| | TGTTTGTGTCACGAAAAAAAAAAAAAAAAAAAAAAAAAAAAAAAAAA | |
| | AAAAAAAAAAA | |
| NM_005228 | CCCCGGCGCAGCGCGGCCGCAGCAGCCTCCGCCCCCCGCACGGTGTGA | 163 |
| | GCGCCCGACGCGGCCGAGGCGGCCGGAGTCCCGAGCTAGCCCCGGCGG | |
| | CCGCCGCCGCCCAGACCGGACGACAGGCCACCTCGTCGGCGTCCGCCC | |
| | GAGTCCCCGCCTCGCCGCCAACGCCACAACCACCGCGCACGGCCCCCTG | |
| | ACTCCGTCCAGTATTGATCGGGAGAGCCGGAGCGAGCTCTTCGGGGAG | |
| | CAGCGATGCGACCCTCCGGGACGGCCGGGGCAGCGCTCCTGGCGCTGC | |
| | TGGCTGCGCTCTGCCCGGCGAGTCGGGCTCTGGAGGAAAAGAAAGTTT | |
| | GCCAAGGCACGAGTAACAAGCTCACGCAGTTGGGCACTTTTGAAGATC | |
| | ATTTTCTCAGCCTCCAGAGGATGTTCAATAACTGTGAGGTGGTCCTTGG | |
| | GAATTTGGAAATTACCTATGTGCAGAGGAATTATGATCTTTCCTTCTTA | |
| | AAGACCATCCAGGAGGTGGCTGGTTATGTCCTCATTGCCCTCAACACAG | |
| | TGGAGCGAATTCCTTTGGAAAACCTGCAGATCATCAGAGGAAATATGT | |
| | ACTACGAAAATTCCTATGCCTTAGCAGTCTTATCTAACTATGATGCAAA | |
| | TAAAACCGGACTGAAGGAGCTGCCCATGAGAAATTTACAGGAAATCCT | |
| | GCATGGCGCCGTGCGGTTCAGCAACAACCCTGCCCTGTGCAACGTGGA | |
| | GAGCATCCAGTGGCGGGACATAGTCAGCAGTGACTTTCTCAGCAACAT | |
| | GTCGATGGACTTCCAGAACCACCTGGGCAGCTGCCAAAAGTGTGATCC | |
| | AAGCTGTCCCAATGGGAGCTGCTGGGGTGCAGGAGAGGAGAACTGCCA | |
| | GAAACTGACCAAAATCATCTGTGCCCAGCAGTGCTCCGGGCGCTGCCGT | |
| | GGCAAGTCCCCCAGTGACTGCTGCCACAACCAGTGTGCTGCAGGCTGCA | |
| | CAGGCCCCCGGGAGAGCGACTGCCTGGTCTGCCGCAAATTCCGAGACG | |
| | AAGCCACGTGCAAGGACACCTGCCCCCCACTCATGCTCTACAACCCCAC | |
| | CACGTACCAGATGGATGTGAACCCCGAGGGCAAATACAGCTTTGGTGC | |
| | CACCTGCGTGAAGAAGTGTCCCCGTAATTATGTGGTGACAGATCACGGC | |
| | TCGTGCGTCCGAGCCTGTGGGGCCGACAGCTATGAGATGGAGGAAGAC | |
| | GGCGTCCGCAAGTGTAAGAAGTGCGAAGGGCCTTGCCGCAAAGTGTGT | |
| | AACGGAATAGGTATTGGTGAATTTAAAGACTCACTCTCCATAAATGCTA | |
| | CGAATATTAAACACTTCAAAAACTGCACCTCCATCAGTGGCGATCTCCA | |
| | CATCCTGCCGGTGGCATTTAGGGGTGACTCCTTCACACATACTCCTCCTC | |
| | TGGATCCACAGGAACTGGATATTCTGAAAACCGTAAAGGAAATCACAG | |
| | GGTTTTTGCTGATTCAGGCTTGGCCTGAAAACAGGACGGACCTCCATGC | |
| | CTTTGAGAACCTAGAAATCATACGCGGCAGGACCAAGCAACATGGTCA | |
| | GTTTTCTCTTGCAGTCGTCAGCCTGAACATAACATCCTTGGGATTACGCT | |
| | CCCTCAAGGAGATAAGTGATGGAGATGTGATAATTTCAGGAAACAAAA | |
| | ATTTGTGCTATGCAAATACAATAAACTGGAAAAAACTGTTTGGGACCTC | |
| | CGGTCAGAAAACCAAAATTATAAGCAACAGAGGTGAAAACAGCTGCAA | |
| | GGCCACAGGCCAGGTCTGCCATGCCTTGTGCTCCCCCGAGGGCTGCTGG | |
| | GGCCCGGAGCCCAGGGACTGCGTCTCTTGCCGGAATGTCAGCCGAGGC | |
| | AGGGAATGCGTGGACAAGTGCAACCTTCTGGAGGGTGAGCCAAGGGAG | |
| | TTTGTGGAGAACTCTGAGTGCATACAGTGCCACCCAGAGTGCCTGCCTC | |
| | AGGCCATGAACATCACCTGCACAGGACGGGGACCAGACAACTGTATCC | |
| | AGTGTGCCCACTACATTGACGGCCCCCACTGCGTCAAGACCTGCCCGGC | |
| | AGGAGTCATGGGAGAAAACAACACCCTGGTCTGGAAGTACGCAGACGC | |
| | CGGCCATGTGTGCCACCTGTGCCATCCAAACTGCACCTACGGATGCACT | |
| | GGGCCAGGTCTTGAAGGCTGTCCAACGAATGGGCCTAAGATCCCGTCC | |
| | ATCGCCACTGGGATGGTGGGGGCCCTCCTCTTGCTGCTGGTGGTGGCCC | |
| | TGGGGATCGGCCTCTTCATGCGAAGGCGCCACATCGTTCGGAAGCGCAC | |
| | GCTGCGGAGGCTGCTGCAGGAGAGGGAGCTTGTGGAGCCTCTTACACC | |
| | CAGTGGAGAAGCTCCCAACCAAGCTCTCTTGAGGATCTTGAAGGAAAC | |
| | TGAATTCAAAAAGATCAAAGTGCTGGGCTCCGGTGCGTTCGGCACGGT | |
| | GTATAAGGGACTCTGGATCCCAGAAGGTGAGAAAGTTAAAATTCCCGT | |
| | CGCTATCAAGGAATTAAGAGAAGCAACATCTCCGAAAGCCAACAAGGA | |
| | AATCCTCGATGAAGCCTACGTGATGGCCAGCGTGGACAACCCCCACGT | |
| | GTGCCGCCTGCTGGGCATCTGCCTCACCTCCACCGTGCAGCTCATCACG | |
| | CAGCTCATGCCCTTCGGCTGCCTCCTGGACTATGTCCGGGAACACAAAG | |
| | ACAATATTGGCTCCCAGTACCTGCTCAACTGGTGTGTGCAGATCGCAAA | |
| | GGGCATGAACTACTTGGAGGACCGTCGCTTGGTGCACCGCGACCTGGC | |
| | AGCCAGGAACGTACTGGTGAAAACACCGCAGCATGTCAAGATCACAGA | |
| | TTTTGGGCTGGCCAAACTGCTGGGTGCGGAAGAGAAAGAATACCATGC | |
| | AGAAGGAGGCAAAGTGCCTATCAAGTGGATGGCATTGGAATCAATTTT | |
| | ACACAGAATCTATACCCACCAGAGTGATGTCTGGAGCTACGGGGTGAC | |
| | CGTTTGGGAGTTGATGACCTTTGGATCCAAGCCATATGACGGAATCCCT | |
| | GCCAGCGAGATCTCCTCCATCCTGGAGAAAGGAGAACGCCTCCCTCAG | |
| | CCACCCATATGTACCATCGATGTCTACATGATCATGGTCAAGTGCTGGA | |
| | TGATAGACGCAGATAGTCGCCCAAAGTTCCGTGAGTTGATCATCGAATT | |
| | CTCCAAAATGGCCCGAGACCCCCAGCGCTACCTTGTCATTCAGGGGGAT | |
| | GAAAGAATGCATTTGCCAAGTCCTACAGACTCCAACTTCTACCGTGCCC | |
| | TGATGGATGAAGAAGACATGGACGACGTGGTGGATGCCGACGAGTACC | |
| | TCATCCCACAGCAGGGCTTCTTCAGCAGCCCCTCCACGTCACGGACTCC | |
| | CCTCCTGAGCTCTCTGAGTGCAACCAGCAACAATTCCACCGTGGCTTGC | |
| | ATTGATAGAAATGGGCTGCAAAGCTGTCCCATCAAGGAAGACAGCTTC | |
| | TTGCAGCGATACAGCTCAGACCCCACAGGCGCCTTGACTGAGGACAGC | |
| | ATAGACGACACCTTCCTCCCAGTGCCTGAATACATAAACCAGTCCGTTC | |
| | CCAAAAGGCCCGCTGGCTCTGTGCAGAATCCTGTCTATCACAATCAGCC | |
| | TCTGAACCCCGCGCCCAGCAGAGACCCACACTACCAGGACCCCCACAG | |
| | CACTGCAGTGGGCAACCCCGAGTATCTCAACACTGTCCAGCCCACCTGT | |
| | GTCAACAGCACATTCGACAGCCCTGCCCACTGGGCCCAGAAAGGCAGC | |
| | CACCAAATTAGCCTGGACAACCCTGACTACCAGCAGGACTTCTTTCCCA | |
| | AGGAAGCCAAGCCAAATGGCATCTTTAAGGGCTCCACAGCTGAAAATG | |
| | CAGAATACCTAAGGGTCGCGCCACAAAGCAGTGAATTTATTGGAGCAT | |
| | GACCACGGAGGATAGTATGAGCCCTAAAAATCCAGACTCTTTCGATACC | |
| | CAGGACCAAGCCACAGCAGGTCCTCCATCCCAACAGCCATGCCCGCATT | |
| | AGCTCTTAGACCCACAGACTGGTTTTGCAACGTTTACACCGACTAGCCA | |
| | GGAAGTACTTCCACCTCGGGCACATTTTGGGAAGTTGCATTCCTTTGTCT | |
| | TCAAACTGTGAAGCATTTACAGAAACGCATCCAGCAAGAATATTGTCCC | |
| | TTTGAGCAGAAATTTATCTTTCAAAGAGGTATATTTGAAAAAAAAAAAA | |
| | AGTATATGTGAGGATTTTTATTGATTGGGGATCTTGGAGTTTTTCATTGT | |
| | CGCTATTGATTTTTACTTCAATGGGCTCTTCCAACAAGGAAGAAGCTTG | |
| | CTGGTAGCACTTGCTACCCTGAGTTCATCCAGGCCCAACTGTGAGCAAG | |
| | GAGCACAAGCCACAAGTCTTCCAGAGGATGCTTGATTCCAGTGGTTCTG | |
| | CTTCAAGGCTTCCACTGCAAAACACTAAAGATCCAAGAAGGCCTTCATG | |
| | GCCCCAGCAGGCCGGATCGGTACTGTATCAAGTCATGGCAGGTACAGT | |
| | AGGATAAGCCACTCTGTCCCTTCCTGGGCAAAGAAGAAACGGAGGGGA | |
| | TGGAATTCTTCCTTAGACTTACTTTTGTAAAAATGTCCCCACGGTACTTA | |
| | CTCCCCACTGATGGACCAGTGGTTTCCAGTCATGAGCGTTAGACTGACT | |
| | TGTTTGTCTTCCATTCCATTGTTTTGAAACTCAGTATGCTGCCCCTGTCTT | |
| | GCTGTCATGAAATCAGCAAGAGAGGATGACACATCAAATAATAACTCG | |
| | GATTCCAGCCCACATTGGATTCATCAGCATTTGGACCAATAGCCCACAG | |
| | CTGAGAATGTGGAATACCTAAGGATAGCACCGCTTTTGTTCTCGCAAAA | |
| | ACGTATCTCCTAATTTGAGGCTCAGATGAAATGCATCAGGTCCTTTGGG | |
| | GCATAGATCAGAAGACTACAAAAATGAAGCTGCTCTGAAATCTCCTTTA | |
| | GCCATCACCCCAACCCCCCAAAATTAGTTTGTGTTACTTATGGAAGATA | |
| | GTTTTCTCCTTTTACTTCACTTCAAAAGCTTTTTACTCAAAGAGTATATG | |
| | TTCCCTCCAGGTCAGCTGCCCCCAAACCCCCTCCTTACGCTTTGTCACAC | |
| | AAAAAGTGTCTCTGCCTTGAGTCATCTATTCAAGCACTTACAGCTCTGG | |
| | CCACAACAGGGCATTTTACAGGTGCGAATGACAGTAGCATTATGAGTA | |
| | GTGTGGAATTCAGGTAGTAAATATGAAACTAGGGTTTGAAATTGATAAT | |
| | GCTTTCACAACATTTGCAGATGTTTTAGAAGGAAAAAAGTTCCTTCCTA | |
| | AAATAATTTCTCTACAATTGGAAGATTGGAAGATTCAGCTAGTTAGGAG | |
| | CCCACCTTTTTTCCTAATCTGTGTGTGCCCTGTAACCTGACTGGTTAACA | |
| | GCAGTCCTTTGTAAACAGTGTTTTAAACTCTCCTAGTCAATATCCACCCC | |
| | ATCCAATTTATCAAGGAAGAAATGGTTCAGAAAATATTTTCAGCCTACA | |
| | GTTATGTTCAGTCACACACACATACAAAATGTTCCTTTTGCTTTTAAAGT | |
| | AATTTTTGACTCCCAGATCAGTCAGAGCCCCTACAGCATTGTTAAGAAA | |
| | GTATTTGATTTTTGTCTCAATGAAAATAAAACTATATTCATTTCCACTCT | |
| | AAAAAAAAAAAAAAAAA | |
| NM_001005862 | GTTCCCGGATTTTTGTGGGCGCCTGCCCCGCCCCTCGTCCCCCTGCTGTG | 164 |
| | TCCATATATCGAGGCGATAGGGTTAAGGGAAGGCGGACGCCTGATGGG | |
| | TTAATGAGCAAACTGAAGTGTTTTCCATGATCTTTTTTGAGTCGCAATTG | |
| | AAGTACCACCTCCCGAGGGTGATTGCTTCCCCATGCGGGGTAGAACCTT | |
| | TGCTGTCCTGTTCACCACTCTACCTCCAGCACAGAATTTGGCTTATGCCT | |
| | ACTCAATGTGAAGATGATGAGGATGAAAACCTTTGTGATGATCCACTTC | |
| | CACTTAATGAATGGTGGCAAAGCAAAGCTATATTCAAGACCACATGCA | |
| | AAGCTACTCCCTGAGCAAAGAGTCACAGATAAAACGGGGGCACCAGTA | |
| | GAATGGCCAGGACAAACGCAGTGCAGCACAGAGACTCAGACCCTGGCA | |
| | GCCATGCCTGCGCAGGCAGTGATGAGAGTGACATGTACTGTTGTGGAC | |
| | ATGCACAAAAGTGAGTGTGCACCGGCACAGACATGAAGCTGCGGCTCC | |
| | CTGCCAGTCCCGAGACCCACCTGGACATGCTCCGCCACCTCTACCAGGG | |
| | CTGCCAGGTGGTGCAGGGAAACCTGGAACTCACCTACCTGCCCACCAAT | |
| | GCCAGCCTGTCCTTCCTGCAGGATATCCAGGAGGTGCAGGGCTACGTGC | |
| | TCATCGCTCACAACCAAGTGAGGCAGGTCCCACTGCAGAGGCTGCGGA | |
| | TTGTGCGAGGCACCCAGCTCTTTGAGGACAACTATGCCCTGGCCGTGCT | |
| | AGACAATGGAGACCCGCTGAACAATACCACCCCTGTCACAGGGGCCTC | |
| | CCCAGGAGGCCTGCGGGAGCTGCAGCTTCGAAGCCTCACAGAGATCTT | |
| | GAAAGGAGGGGTCTTGATCCAGCGGAACCCCCAGCTCTGCTACCAGGA | |
| | CACGATTTTGTGGAAGGACATCTTCCACAAGAACAACCAGCTGGCTCTC | |
| | ACACTGATAGACACCAACCGCTCTCGGGCCTGCCACCCCTGTTCTCCGA | |
| | TGTGTAAGGGCTCCCGCTGCTGGGGAGAGAGTTCTGAGGATTGTCAGA | |
| | GCCTGACGCGCACTGTCTGTGCCGGTGGCTGTGCCCGCTGCAAGGGGCC | |
| | ACTGCCCACTGACTGCTGCCATGAGCAGTGTGCTGCCGGCTGCACGGGC | |
| | CCCAAGCACTCTGACTGCCTGGCCTGCCTCCACTTCAACCACAGTGGCA | |
| | TCTGTGAGCTGCACTGCCCAGCCCTGGTCACCTACAACACAGACACGTT | |
| | TGAGTCCATGCCCAATCCCGAGGGCCGGTATACATTCGGCGCCAGCTGT | |
| | GTGACTGCCTGTCCCTACAACTACCTTTCTACGGACGTGGGATCCTGCA | |
| | CCCTCGTCTGCCCCCTGCACAACCAAGAGGTGACAGCAGAGGATGGAA | |
| | CACAGCGGTGTGAGAAGTGCAGCAAGCCCTGTGCCCGAGTGTGCTATG | |
| | GTCTGGGCATGGAGCACTTGCGAGAGGTGAGGGCAGTTACCAGTGCCA | |
| | ATATCCAGGAGTTTGCTGGCTGCAAGAAGATCTTTGGGAGCCTGGCATT | |
| | TCTGCCGGAGAGCTTTGATGGGGACCCAGCCTCCAACACTGCCCCGCTC | |
| | CAGCCAGAGCAGCTCCAAGTGTTTGAGACTCTGGAAGAGATCACAGGT | |
| | TACCTATACATCTCAGCATGGCCGGACAGCCTGCCTGACCTCAGCGTCT | |
| | TCCAGAACCTGCAAGTAATCCGGGGACGAATTCTGCACAATGGCGCCT | |
| | ACTCGCTGACCCTGCAAGGGCTGGGCATCAGCTGGCTGGGGCTGCGCTC | |
| | ACTGAGGGAACTGGGCAGTGGACTGGCCCTCATCCACCATAACACCCA | |
| | CCTCTGCTTCGTGCACACGGTGCCCTGGGACCAGCTCTTTCGGAACCCG | |
| | CACCAAGCTCTGCTCCACACTGCCAACCGGCCAGAGGACGAGTGTGTG | |
| | GGCGAGGGCCTGGCCTGCCACCAGCTGTGCGCCCGAGGGCACTGCTGG | |
| | GGTCCAGGGCCCACCCAGTGTGTCAACTGCAGCCAGTTCCTTCGGGGCC | |
| | AGGAGTGCGTGGAGGAATGCCGAGTACTGCAGGGGCTCCCCAGGGAGT | |
| | ATGTGAATGCCAGGCACTGTTTGCCGTGCCACCCTGAGTGTCAGCCCCA | |
| | GAATGGCTCAGTGACCTGTTTTGGACCGGAGGCTGACCAGTGTGTGGCC | |
| | TGTGCCCACTATAAGGACCCTCCCTTCTGCGTGGCCCGCTGCCCCAGCG | |
| | GTGTGAAACCTGACCTCTCCTACATGCCCATCTGGAAGTTTCCAGATGA | |
| | GGAGGGCGCATGCCAGCCTTGCCCCATCAACTGCACCCACTCCTGTGTG | |
| | GACCTGGATGACAAGGGCTGCCCCGCCGAGCAGAGAGCCAGCCCTCTG | |
| | ACGTCCATCATCTCTGCGGTGGTTGGCATTCTGCTGGTCGTGGTCTTGGG | |
| | GGTGGTCTTTGGGATCCTCATCAAGCGACGGCAGCAGAAGATCCGGAA | |
| | GTACACGATGCGGAGACTGCTGCAGGAAACGGAGCTGGTGGAGCCGCT | |
| | GACACCTAGCGGAGCGATGCCCAACCAGGCGCAGATGCGGATCCTGAA | |
| | AGAGACGGAGCTGAGGAAGGTGAAGGTGCTTGGATCTGGCGCTTTTGG | |
| | CACAGTCTACAAGGGCATCTGGATCCCTGATGGGGAGAATGTGAAAAT | |
| | TCCAGTGGCCATCAAAGTGTTGAGGGAAAACACATCCCCCAAAGCCAA | |
| | CAAAGAAATCTTAGACGAAGCATACGTGATGGCTGGTGTGGGCTCCCC | |
| | ATATGTCTCCCGCCTTCTGGGCATCTGCCTGACATCCACGGTGCAGCTG | |
| | GTGACACAGCTTATGCCCTATGGCTGCCTCTTAGACCATGTCCGGGAAA | |
| | ACCGCGGACGCCTGGGCTCCCAGGACCTGCTGAACTGGTGTATGCAGAT | |
| | TGCCAAGGGGATGAGCTACCTGGAGGATGTGCGGCTCGTACACAGGGA | |
| | CTTGGCCGCTCGGAACGTGCTGGTCAAGAGTCCCAACCATGTCAAAATT | |
| | ACAGACTTCGGGCTGGCTCGGCTGCTGGACATTGACGAGACAGAGTAC | |
| | CATGCAGATGGGGGCAAGGTGCCCATCAAGTGGATGGCGCTGGAGTCC | |
| | ATTCTCCGCCGGCGGTTCACCCACCAGAGTGATGTGTGGAGTTATGGTG | |
| | TGACTGTGTGGGAGCTGATGACTTTTGGGGCCAAACCTTACGATGGGAT | |
| | CCCAGCCCGGGAGATCCCTGACCTGCTGGAAAAGGGGGAGCGGCTGCC | |
| | CCAGCCCCCCATCTGCACCATTGATGTCTACATGATCATGGTCAAATGT | |
| | TGGATGATTGACTCTGAATGTCGGCCAAGATTCCGGGAGTTGGTGTCTG | |
| | AATTCTCCCGCATGGCCAGGGACCCCCAGCGCTTTGTGGTCATCCAGAA | |
| | TGAGGACTTGGGCCCAGCCAGTCCCTTGGACAGCACCTTCTACCGCTCA | |
| | CTGCTGGAGGACGATGACATGGGGGACCTGGTGGATGCTGAGGAGTAT | |
| | CTGGTACCCCAGCAGGGCTTCTTCTGTCCAGACCCTGCCCCGGGCGCTG | |
| | GGGGCATGGTCCACCACAGGCACCGCAGCTCATCTACCAGGAGTGGCG | |
| | GTGGGGACCTGACACTAGGGCTGGAGCCCTCTGAAGAGGAGGCCCCCA | |
| | GGTCTCCACTGGCACCCTCCGAAGGGGCTGGCTCCGATGTATTTGATGG | |
| | TGACCTGGGAATGGGGGCAGCCAAGGGGCTGCAAAGCCTCCCCACACA | |
| | TGACCCCAGCCCTCTACAGCGGTACAGTGAGGACCCCACAGTACCCCTG | |
| | CCCTCTGAGACTGATGGCTACGTTGCCCCCCTGACCTGCAGCCCCCAGC | |
| | CTGAATATGTGAACCAGCCAGATGTTCGGCCCCAGCCCCCTTCGCCCCG | |
| | AGAGGGCCCTCTGCCTGCTGCCCGACCTGCTGGTGCCACTCTGGAAAGG | |
| | CCCAAGACTCTCTCCCCAGGGAAGAATGGGGTCGTCAAAGACGTTTTTG | |
| | CCTTTGGGGGTGCCGTGGAGAACCCCGAGTACTTGACACCCCAGGGAG | |
| | GAGCTGCCCCTCAGCCCCACCCTCCTCCTGCCTTCAGCCCAGCCTTCGA | |
| | CAACCTCTATTACTGGGACCAGGACCCACCAGAGCGGGGGGCTCCACC | |
| | CAGCACCTTCAAAGGGACACCTACGGCAGAGAACCCAGAGTACCTGGG | |
| | TCTGGACGTGCCAGTGTGAACCAGAAGGCCAAGTCCGCAGAAGCCCTG | |
| | ATGTGTCCTCAGGGAGCAGGGAAGGCCTGACTTCTGCTGGCATCAAGA | |
| | GGTGGGAGGGCCCTCCGACCACTTCCAGGGGAACCTGCCATGCCAGGA | |
| | ACCTGTCCTAAGGAACCTTCCTTCCTGCTTGAGTTCCCAGATGGCTGGA | |
| | AGGGGTCCAGCCTCGTTGGAAGAGGAACAGCACTGGGGAGTCTTTGTG | |
| | GATTCTGAGGCCCTGCCCAATGAGACTCTAGGGTCCAGTGGATGCCACA | |
| | GCCCAGCTTGGCCCTTTCCTTCCAGATCCTGGGTACTGAAAGCCTTAGG | |
| | GAAGCTGGCCTGAGAGGGGAAGCGGCCCTAAGGGAGTGTCTAAGAACA | |
| | AAAGCGACCCATTCAGAGACTGTCCCTGAAACCTAGTACTGCCCCCCAT | |
| | GAGGAAGGAACAGCAATGGTGTCAGTATCCAGGCTTTGTACAGAGTGC | |
| | TTTTCTGTTTAGTTTTTACTTTTTTTGTTTTGTTTTTTTAAAGATGAAATA | |
| | AAGACCCAGGGGGAGAATGGGTGTTGTATGGGGAGGCAAGTGTGGGGG | |
| | GTCCTTCTCCACACCCACTTTGTCCATTTGCAAATATATTTTGGAAAACA | |
| | GCTA | |
| NM_001122742 | ATGGTCATAACAGCCTCCTGTCTACCGACTCAGAACGGATTTTACCAAA | 165 |
| | ACTGAAAATGCAGGCTCCATGCTCAGAAGCTCTTTAACAGGCTCGAAA | |
| | GGTCCATGCTCCTTTCTCCTGCCCATTCTATAGCATAAGAAGACAGTCTC | |
| | TGAGTGATAATCTTCTCTTCAAGAAGAAGAAAACTAGGAAGGAGTAAG | |
| | CACAAAGATCTCTTCACATTCTCCGGGACTGCGGTACCAAATATCAGCA | |
| | CAGCACTTCTTGAAAAAGGATGTAGATTTTAATCTGAACTTTGAACCAT | |
| | CACTGAGGTGGCCCGCCGGTTTCTGAGCCTTCTGCCCTGCGGGGACACG | |
| | GTCTGCACCCTGCCCGCGGCCACGGACCATGACCATGACCCTCCACACC | |
| | AAAGCATCTGGGATGGCCCTACTGCATCAGATCCAAGGGAACGAGCTG | |
| | GAGCCCCTGAACCGTCCGCAGCTCAAGATCCCCCTGGAGCGGCCCCTGG | |
| | GCGAGGTGTACCTGGACAGCAGCAAGCCCGCCGTGTACAACTACCCCG | |
| | AGGGCGCCGCCTACGAGTTCAACGCCGCGGCCGCCGCCAACGCGCAGG | |
| | TCTACGGTCAGACCGGCCTCCCCTACGGCCCCGGGTCTGAGGCTGCGGC | |
| | GTTCGGCTCCAACGGCCTGGGGGGTTTCCCCCCACTCAACAGCGTGTCT | |
| | CCGAGCCCGCTGATGCTACTGCACCCGCCGCCGCAGCTGTCGCCTTTCC | |
| | TGCAGCCCCACGGCCAGCAGGTGCCCTACTACCTGGAGAACGAGCCCA | |
| | GCGGCTACACGGTGCGCGAGGCCGGCCCGCCGGCATTCTACAGGCCAA | |
| | ATTCAGATAATCGACGCCAGGGTGGCAGAGAAAGATTGGCCAGTACCA | |
| | ATGACAAGGGAAGTATGGCTATGGAATCTGCCAAGGAGACTCGCTACT | |
| | GTGCAGTGTGCAATGACTATGCTTCAGGCTACCATTATGGAGTCTGGTC | |
| | CTGTGAGGGCTGCAAGGCCTTCTTCAAGAGAAGTATTCAAGGACATAA | |
| | CGACTATATGTGTCCAGCCACCAACCAGTGCACCATTGATAAAAACAG | |
| | GAGGAAGAGCTGCCAGGCCTGCCGGCTCCGCAAATGCTACGAAGTGGG | |
| | AATGATGAAAGGTGGGATACGAAAAGACCGAAGAGGAGGGAGAATGT | |
| | TGAAACACAAGCGCCAGAGAGATGATGGGGAGGGCAGGGGTGAAGTG | |
| | GGGTCTGCTGGAGACATGAGAGCTGCCAACCTTTGGCCAAGCCCGCTCA | |
| | TGATCAAACGCTCTAAGAAGAACAGCCTGGCCTTGTCCCTGACGGCCGA | |
| | CCAGATGGTCAGTGCCTTGTTGGATGCTGAGCCCCCCATACTCTATTCC | |
| | GAGTATGATCCTACCAGACCCTTCAGTGAAGCTTCGATGATGGGCTTAC | |
| | TGACCAACCTGGCAGACAGGGAGCTGGTTCACATGATCAACTGGGCGA | |
| | AGAGGGTGCCAGGCTTTGTGGATTTGACCCTCCATGATCAGGTCCACCT | |
| | TCTAGAATGTGCCTGGCTAGAGATCCTGATGATTGGTCTCGTCTGGCGC | |
| | TCCATGGAGCACCCAGGGAAGCTACTGTTTGCTCCTAACTTGCTCTTGG | |
| | ACAGGAACCAGGGAAAATGTGTAGAGGGCATGGTGGAGATCTTCGACA | |
| | TGCTGCTGGCTACATCATCTCGGTTCCGCATGATGAATCTGCAGGGAGA | |
| | GGAGTTTGTGTGCCTCAAATCTATTATTTTGCTTAATTCTGGAGTGTACA | |
| | CATTTCTGTCCAGCACCCTGAAGTCTCTGGAAGAGAAGGACCATATCCA | |
| | CCGAGTCCTGGACAAGATCACAGACACTTTGATCCACCTGATGGCCAAG | |
| | GCAGGCCTGACCCTGCAGCAGCAGCACCAGCGGCTGGCCCAGCTCCTC | |
| | CTCATCCTCTCCCACATCAGGCACATGAGTAACAAAGGCATGGAGCATC | |
| | TGTACAGCATGAAGTGCAAGAACGTGGTGCCCCTCTATGACCTGCTGCT | |
| | GGAGATGCTGGACGCCCACCGCCTACATGCGCCCACTAGCCGTGGAGG | |
| | GGCATCCGTGGAGGAGACGGACCAAAGCCACTTGGCCACTGCGGGCTC | |
| | TACTTCATCGCATTCCTTGCAAAAGTATTACATCACGGGGGAGGCAGAG | |
| | GGTTTCCCTGCCACGGTCTGAGAGCTCCCTGGCTCCCACACGGTTCAGA | |
| | TAATCCCTGCTGCATTTTACCCTCATCATGCACCACTTTAGCCAAATTCT | |
| | GTCTCCTGCATACACTCCGGCATGCATCCAACACCAATGGCTTTCTAGA | |
| | TGAGTGGCCATTCATTTGCTTGCTCAGTTCTTAGTGGCACATCTTCTGTC | |
| | TTCTGTTGGGAACAGCCAAAGGGATTCCAAGGCTAAATCTTTGTAACAG | |
| | CTCTCTTTCCCCCTTGCTATGTTACTAAGCGTGAGGATTCCCGTAGCTCT | |
| | TCACAGCTGAACTCAGTCTATGGGTTGGGGCTCAGATAACTCTGTGCAT | |
| | TTAAGCTACTTGTAGAGACCCAGGCCTGGAGAGTAGACATTTTGCCTCT | |
| | GATAAGCACTTTTTAAATGGCTCTAAGAATAAGCCACAGCAAAGAATTT | |
| | AAAGTGGCTCCTTTAATTGGTGACTTGGAGAAAGCTAGGTCAAGGGTTT | |
| | ATTATAGCACCCTCTTGTATTCCTATGGCAATGCATCCTTTTATGAAAGT | |
| | GGTACACCTTAAAGCTTTTATATGACTGTAGCAGAGTATCTGGTGATTG | |
| | TCAATTCATTCCCCCTATAGGAATACAAGGGGCACACAGGGAAGGCAG | |
| | ATCCCCTAGTTGGCAAGACTATTTTAACTTGATACACTGCAGATTCAGA | |
| | TGTGCTGAAAGCTCTGCCTCTGGCTTTCCGGTCATGGGTTCCAGTTAATT | |
| | CATGCCTCCCATGGACCTATGGAGAGCAGCAAGTTGATCTTAGTTAAGT | |
| | CTCCCTATATGAGGGATAAGTTCCTGATTTTTGTTTTTATTTTTGTGTTAC | |
| | AAAAGAAAGCCCTCCCTCCCTGAACTTGCAGTAAGGTCAGCTTCAGGAC | |
| | CTGTTCCAGTGGGCACTGTACTTGGATCTTCCCGGCGTGTGTGTGCCTTA | |
| | CACAGGGGTGAACTGTTCACTGTGGTGATGCATGATGAGGGTAAATGG | |
| | TAGTTGAAAGGAGCAGGGGCCCTGGTGTTGCATTTAGCCCTGGGGCATG | |
| | GAGCTGAACAGTACTTGTGCAGGATTGTTGTGGCTACTAGAGAACAAG | |
| | AGGGAAAGTAGGGCAGAAACTGGATACAGTTCTGAGGCACAGCCAGAC | |
| | TTGCTCAGGGTGGCCCTGCCACAGGCTGCAGCTACCTAGGAACATTCCT | |
| | TGCAGACCCCGCATTGCCCTTTGGGGGTGCCCTGGGATCCCTGGGGTAG | |
| | TCCAGCTCTTCTTCATTTCCCAGCGTGGCCCTGGTTGGAAGAAGCAGCT | |
| | GTCACAGCTGCTGTAGACAGCTGTGTTCCTACAATTGGCCCAGCACCCT | |
| | GGGGCACGGGAGAAGGGTGGGGACCGTTGCTGTCACTACTCAGGCTGA | |
| | CTGGGGCCTGGTCAGATTACGTATGCCCTTGGTGGTTTAGAGATAATCC | |
| | AAAATCAGGGTTTGGTTTGGGGAAGAAAATCCTCCCCCTTCCTCCCCCG | |
| | CCCCGTTCCCTACCGCCTCCACTCCTGCCAGCTCATTTCCTTCAATTTCC | |
| | TTTGACCTATAGGCTAAAAAAGAAAGGCTCATTCCAGCCACAGGGCAG | |
| | CCTTCCCTGGGCCTTTGCTTCTCTAGCACAATTATGGGTTACTTCCTTTTT | |
| | CTTAACAAAAAAGAATGTTTGATTTCCTCTGGGTGACCTTATTGTCTGTA | |
| | ATTGAAACCCTATTGAGAGGTGATGTCTGTGTTAGCCAATGACCCAGGT | |
| | GAGCTGCTCGGGCTTCTCTTGGTATGTCTTGTTTGGAAAAGTGGATTTCA | |
| | TTCATTTCTGATTGTCCAGTTAAGTGATCACCAAAGGACTGAGAATCTG | |
| | GGAGGGCAAAAAAAAAAAAAAAGTTTTTATGTGCACTTAAATTTGGGG | |
| | ACAATTTTATGTATCTGTGTTAAGGATATGTTTAAGAACATAATTCTTTT | |
| | GTTGCTGTTTGTTTAAGAAGCACCTTAGTTTGTTTAAGAAGCACCTTATA | |
| | TAGTATAATATATATTTTTTTGAAATTACATTGCTTGTTTATCAGACAAT | |
| | TGAATGTAGTAATTCTGTTCTGGATTTAATTTGACTGGGTTAACATGCA | |
| | AAAACCAAGGAAAAATATTTAGTTTTTTTTTTTTTTTTTGTATACTTTTC | |
| | AAGCTACCTTGTCATGTATACAGTCATTTATGCCTAAAGCCTGGTGATT | |
| | ATTCATTTAAATGAAGATCACATTTCATATCAACTTTTGTATCCACAGTA | |
| | GACAAAATAGCACTAATCCAGATGCCTATTGTTGGATACTGAATGACAG | |
| | ACAATCTTATGTAGCAAAGATTATGCCTGAAAAGGAAAATTATTCAGG | |
| | GCAGCTAATTTTGCTTTTACCAAAATATCAGTAGTAATATTTTTGGACA | |
| | GTAGCTAATGGGTCAGTGGGTTCTTTTTAATGTTTATACTTAGATTTTCT | |
| | TTTAAAAAAATTAAAATAAAACAAAAAAAAATTTCTAGGACTAGACGA | |
| | TGTAATACCAGCTAAAGCCAAACAATTATACAGTGGAAGGTTTTACATT | |
| | ATTCATCCAATGTGTTTCTATTCATGTTAAGATACTACTACATTTGAAGT | |
| | GGGCAGAGAACATCAGATGATTGAAATGTTCGCCCAGGGGTCTCCAGC | |
| | AACTTTGGAAATCTCTTTGTATTTTTACTTGAAGTGCCACTAATGGACAG | |
| | CAGATATTTTCTGGCTGATGTTGGTATTGGGTGTAGGAACATGATTTAA | |
| | AAAAAAACTCTTGCCTCTGCTTTCCCCCACTCTGAGGCAAGTTAAAATG | |
| | TAAAAGATGTGATTTATCTGGGGGGCTCAGGTATGGTGGGGAAGTGGA | |
| | TTCAGGAATCTGGGGAATGGCAAATATATTAAGAAGAGTATTGAAAGT | |
| | ATTTGGAGGAAAATGGTTAATTCTGGGTGTGCACCAGGGTTCAGTAGAG | |
| | TCCACTTCTGCCCTGGAGACCACAAATCAACTAGCTCCATTTACAGCCA | |
| | TTTCTAAAATGGCAGCTTCAGTTCTAGAGAAGAAAGAACAACATCAGC | |
| | AGTAAAGTCCATGGAATAGCTAGTGGTCTGTGTTTCTTTTCGCCATTGCC | |
| | TAGCTTGCCGTAATGATTCTATAATGCCATCATGCAGCAATTATGAGAG | |
| | GCTAGGTCATCCAAAGAGAAGACCCTATCAATGTAGGTTGCAAAATCT | |
| | AACCCCTAAGGAAGTGCAGTCTTTGATTTGATTTCCCTAGTAACCTTGC | |
| | AGATATGTTTAACCAAGCCATAGCCCATGCCTTTTGAGGGCTGAACAAA | |
| | TAAGGGACTTACTGATAATTTACTTTTGATCACATTAAGGTGTTCTCACC | |
| | TTGAAATCTTATACACTGAAATGGCCATTGATTTAGGCCACTGGCTTAG | |
| | AGTACTCCTTCCCCTGCATGACACTGATTACAAATACTTTCCTATTCATA | |
| | CTTTCCAATTATGAGATGGACTGTGGGTACTGGGAGTGATCACTAACAC | |
| | CATAGTAATGTCTAATATTCACAGGCAGATCTGCTTGGGGAAGCTAGTT | |
| | ATGTGAAAGGCAAATAGAGTCATACAGTAGCTCAAAAGGCAACCATAA | |
| | TTCTCTTTGGTGCAGGTCTTGGGAGCGTGATCTAGATTACACTGCACCA | |
| | TTCCCAAGTTAATCCCCTGAAAACTTACTCTCAACTGGAGCAAATGAAC | |
| | TTTGGTCCCAAATATCCATCTTTTCAGTAGCGTTAATTATGCTCTGTTTC | |
| | CAACTGCATTTCCTTTCCAATTGAATTAAAGTGTGGCCTCGTTTTTAGTC | |
| | ATTTAAAATTGTTTTCTAAGTAATTGCTGCCTCTATTATGGCACTTCAAT | |
| | TTTGCACTGTCTTTTGAGATTCAAGAAAAATTTCTATTCTTTTTTTTGCAT | |
| | CCAATTGTGCCTGAACTTTTAAAATATGTAAATGCTGCCATGTTCCAAA | |
| | CCCATCGTCAGTGTGTGTGTTTAGAGCTGTGCACCCTAGAAACAACATA | |
| | TTGTCCCATGAGCAGGTGCCTGAGACACAGACCCCTTTGCATTCACAGA | |
| | GAGGTCATTGGTTATAGAGACTTGAATTAATAAGTGACATTATGCCAGT | |
| | TTCTGTTCTCTCACAGGTGATAAACAATGCTTTTTGTGCACTACATACTC | |
| | TTCAGTGTAGAGCTCTTGTTTTATGGGAAAAGGCTCAAATGCCAAATTG | |
| | TGTTTGATGGATTAATATGCCCTTTTGCCGATGCATACTATTACTGATGT | |
| | GACTCGGTTTTGTCGCAGCTTTGCTTTGTTTAATGAAACACACTTGTAAA | |
| | CCTCTTTTGCACTTTGAAAAAGAATCCAGCGGGATGCTCGAGCACCTGT | |
| | AAACAATTTTCTCAACCTATTTGATGTTCAAATAAAGAATTAAACTAAA | |
| NM_130398 | AAATTGAAAGGTCAGCCTTTCGCGCGCTGTGTAGGCAAGTTACCCGTGT | 166 |
| | TCTGCGTTGCCGGCCGTGGGTGCTCTGGCCACAGTGAGTTAGGGGCGTC | |
| | GGAGCGGGTTTCTCCAACCGCAATCGGCTCCGCTCAAGGGGAGGAGGA | |
| | GAGTCCCTTCTCGGAAGGCCTAAGGAAACGTGTCGTCTGGAATGGGCTT | |
| | GGGGGCCACGCCTGCACATCTCCGCGAGACAGAGGGATAAAGTGAAGA | |
| | TGGTGCTGTTATTGTTACCTCGAGTGCCACATGCGACCTCTGAGATATG | |
| | TACACAGTCATTCTTACTATCGCACTCAGCCATTCTTACTACGCTAAAG | |
| | AAGAAATAATTATTCGAGGATATTTGCCTGGCCCAGAAGAAACTTATGT | |
| | AAATTTCATGAACTATTATATCCGTTTTCCTCGGAGTGAGAGAAAACTC | |
| | TTTTTAGATATCATCTGAGAGAACTAGTGAATCCCAGTCACTGAGTGGA | |
| | GTTGAGAGTCTAAGAACCTCTGAAATTTGAGAACTGCTGGACCAGAGC | |
| | CTTTAGAGCTCTGATAAGGTGTCAACAGGGTAGTTAATTTGGCACCATG | |
| | GGGATACAGGGATTGCTACAATTTATCAAAGAAGCTTCAGAACCCATCC | |
| | ATGTGAGGAAGTATAAAGGGCAGGTAGTAGCTGTGGATACATATTGCT | |
| | GGCTTCACAAAGGAGCTATTGCTTGTGCTGAAAAACTAGCCAAAGGTG | |
| | AACCTACTGATAGGTATGTAGGATTTTGTATGAAATTTGTAAATATGTT | |
| | ACTATCTCATGGGATCAAGCCTATTCTCGTATTTGATGGATGTACTTTAC | |
| | CTTCTAAAAAGGAAGTAGAGAGATCTAGAAGAGAAAGACGACAAGCC | |
| | AATCTTCTTAAGGGAAAGCAACTTCTTCGTGAGGGGAAAGTCTCGGAA | |
| | GCTCGAGAGTGTTTCACCCGGTCTATCAATATCACACATGCCATGGCCC | |
| | ACAAAGTAATTAAAGCTGCCCGGTCTCAGGGGGTAGATTGCCTCGTGGC | |
| | TCCCTATGAAGCTGATGCGCAGTTGGCCTATCTTAACAAAGCGGGAATT | |
| | GTGCAAGCCATAATTACAGAGGACTCGGATCTCCTAGCTTTTGGCTGTA | |
| | AAAAGGTAATTTTAAAGATGGACCAGTTTGGAAATGGACTTGAAATTG | |
| | ATCAAGCTCGGCTAGGAATGTGCAGACAGCTTGGGGATGTATTCACGG | |
| | AAGAGAAGTTTCGTTACATGTGTATTCTTTCAGGTTGTGACTACCTGTCA | |
| | TCACTGCGTGGGATTGGATTAGCAAAGGCATGCAAAGTCCTAAGACTA | |
| | GCCAATAATCCAGATATAGTAAAGGTTATCAAGAAAATTGGACATTATC | |
| | TCAAGATGAATATCACGGTACCAGAGGATTACATCAACGGGTTTATTCG | |
| | GGCCAACAATACCTTCCTCTATCAGCTAGTTTTTGATCCCATCAAAAGG | |
| | AAACTTATTCCTCTGAACGCCTATGAAGATGATGTTGATCCTGAAACAC | |
| | TAAGCTACGCTGGGCAATATGTTGATGATTCCATAGCTCTTCAAATAGC | |
| | ACTTGGAAATAAAGATATAAATACTTTTGAACAGATCGATGACTACAAT | |
| | CCAGACACTGCTATGCCTGCCCATTCAAGAAGTCATAGTTGGGATGACA | |
| | AAACATGTCAAAAGTCAGCTAATGTTAGCAGCATTTGGCATAGGAATTA | |
| | CTCTCCCAGACCAGAGTCGGGTACTGTTTCAGATGCCCCACAATTGAAG | |
| | GAAAATCCAAGTACTGTGGGAGTGGAACGAGTGATTAGTACTAAAGGG | |
| | TTAAATCTCCCAAGGAAATCATCCATTGTGAAAAGACCAAGAAGTGCA | |
| | GAGCTGTCAGAAGATGACCTGTTGAGTCAGTATTCTCTTTCATTTACGA | |
| | AGAAGACCAAGAAAAATAGCTCTGAAGGCAATAAATCATTGAGCTTTT | |
| | CTGAAGTGTTTGTGCCTGACCTGGTAAATGGACCTACTAACAAAAAGAG | |
| | TGTAAGCACTCCACCTAGGACGAGAAATAAATTTGCAACATTTTTACAA | |
| | AGGAAAAATGAAGAAAGTGGTGCAGTTGTGGTTCCAGGGACCAGAAGC | |
| | AGGTTTTTTTGCAGTTCAGATTCTACTGACTGTGTATCAAACAAAGTGA | |
| | GCATCCAGCCTCTGGATGAAACTGCTGTCACAGATAAAGAGAACAATC | |
| | TGCATGAATCAGAGTATGGAGACCAAGAAGGCAAGAGACTGGTTGACA | |
| | CAGATGTAGCACGTAATTCAAGTGATGACATTCCGAATAATCATATTCC | |
| | AGGTGATCATATTCCAGACAAGGCAACAGTGTTTACAGATGAAGAGTC | |
| | CTACTCTTTTGAGAGCAGCAAATTTACAAGGACCATTTCACCACCCACT | |
| | TTGGGAACACTAAGAAGTTGTTTTAGTTGGTCTGGAGGTCTTGGAGATT | |
| | TTTCAAGAACGCCGAGCCCCTCTCCAAGCACAGCATTGCAGCAGTTCCG | |
| | AAGAAAGAGCGATTCCCCCACCTCTTTGCCTGAGAATAATATGTCTGAT | |
| | GTGTCGCAGTTAAAGAGCGAGGAGTCCAGTGACGATGAGTCTCATCCCT | |
| | TACGAGAAGAGGCATGTTCTTCACAGTCCCAGGAAAGTGGAGAATTCT | |
| | CACTGCAGAGTTCAAATGCATCAAAGCTTTCTCAGTGCTCTAGTAAGGA | |
| | CTCTGATTCAGAGGAATCTGATTGCAATATTAAGTTACTTGACAGTCAA | |
| | AGTGACCAGACCTCCAAGCTACGTTTATCTCATTTCTCAAAAAAAGACA | |
| | CACCTCTAAGGAACAAGGTTCCTGGGCTATATAAGTCCAGTTCTGCAGA | |
| | CTCTCTTTCTACAACCAAGATCAAACCTCTAGGACCTGCCAGAGCCAGT | |
| | GGGCTGAGCAAGAAGCCGGCAAGCATCCAGAAGAGAAAGCATCATAAT | |
| | GCCGAGAACAAGCCGGGGTTACAGATCAAACTCAATGAGCTCTGGAAA | |
| | AACTTTGGATTTAAAAAAGATTCTGAAAAGCTTCCTCCTTGTAAGAAAC | |
| | CCCTGTCCCCAGTCAGAGATAACATCCAACTAACTCCAGAAGCGGAAG | |
| | AGGATATATTTAACAAACCTGAATGTGGCCGTGTTCAAAGAGCAATATT | |
| | CCAGTAAATGCAGACTGCTGCAAAGCTTTTGCCTGCAAGAGAATCTGAT | |
| | CAATTTGAAGTCCCTGTTTGGGAATGAGGCACTTATCAGCATGAAGAAT | |
| | TTTTTCTCATTCTGTGCCATTTTAAAAATAGAATACATTTTGTATATTAA | |
| | CTTTATAATTGGGTTGTGGTTTTTTTGCTCAGCTTTTTATATTTTTATAAG | |
| | AAGCTAAATAGAAGAATAATTGTATCTCTGACAGGTTTTTGGAGGTTTT | |
| | AGTGTTAATTGGGAAAATCCTCTGGAGTTTATAAAAGTCTACTCTAAAT | |
| | ATTTCTGTAATGTTGTCAAGTAGAAAGATAGTAAATGGAGAAACTACA | |
| | AAAAAAAAAAAAAAAAA | |
| AB209631 | CCATGACCTGCCTTGAGAAGGGGCAGGGGAAGCCAGATGGACTGGAAG | 167 |
| | TGGAGTGGCAGTGACCAAGGAGGAGGAGGTGTGATAGGCTTCCCACGC | |
| | AGGGTAGATCCAGAGACACCAGTGCCACCCATAGGCCCCTAGGACTGC | |
| | AGTGGTCACCCGATTCCTTTGTCCCAGCTGAGACTCAGTTCTGAGTGTTC | |
| | TATTTTGGGGAACAGAGGCGTCCTTGGTAGCATTTGGAAGAGGATAGCC | |
| | AGCTGGGGTGTGTGTACATCACAGCCTGACAGTAACAGCATCCGAACC | |
| | AGAGGTGACTGGCTAAGGGCAGACCCAGGGCAACAGGTTAACCGTTCT | |
| | AGGGCCGGGCACAGGGAGGAGAACATTCCAACACTCTGTGTGCCCAGT | |
| | GCCGACGCACGTTCTCTCTTTTATCCTCAAAACAGTCCTATGAGGATAT | |
| | AAGCCAGAGAGAGACAGAGACAAGGAATTACAAGTTGGTGAGAGTCA | |
| | GGATTTGAACTTGGCTCTGGCAGATGGAAAATTAGGGTCTGTATTCTTT | |
| | ACAAAACCGTGTGTGCCTCAGATGGAGTTGGTGCATAACAAGCAGAGG | |
| | TATCCAGGGTCGCGGTCCTGCTTGCCACGGAAGGGGCCGCCTTGTCAGT | |
| | TGTGACCACCCAGCCCTGGAAATGTCAGTAATGCTGTAAGGAGTGGGG | |
| | ATCGGATCAGATGCCATCCAGATGCTGAAGTTTGACCTTGTGTCATTTTT | |
| | CACTTTCTTTTTTGGCTCTTCTGCAATCAATTCATTTATTTAGCAAAAAA | |
| | GAAATTATGTGTGCCGAGAGCATGCAGAAGATATGTCTCCGTTCTCTGC | |
| | TTCCCTCCAAAAAAGAATCCCAAAACTGCTTTCTGTGAACGTGTGCCAG | |
| | GGTCCCAGCAGGACTCAGGGAGAGCAGGAAGCCCAGCCCAGACCCCTT | |
| | GCACAACCTACCGTGGGGAGGCCTTAGGCTCTGGCTACTACAGAGCTG | |
| | GTTCCAGTCTGCACTGCCACAGCCTGGCCAGGGACTTGGACACATCTGC | |
| | TGGCCACTTCCTGTCTCAGTTTCCTTATCTGCAAAATAAGGGAAAAGCC | |
| | CCCACAAAGGTGCACGTGTAGCAGGAGCTCTTTTCCCTCCCTATTTTAG | |
| | GAAGGCAGTTGGTGGGAAGTCCAGCTTGGGTCCCTGAGAGCTGTGAGA | |
| | AGGAGATGCGGCTGCTGCTGGCCCTGTTGGGGGTCCTGCTGAGTGTGCC | |
| | TGGGCCTCCAGTCTTGTCCCTGGAGGCCTCTGAGGAAGTGGAGCTTGGT | |
| | ATGGCTTCTGAGGTGGGAGAGGGTGGCAGGGGTGGGAAGAGTGGGCAC | |
| | CAGGAGGGGGCTGCTGGGCTGAGCAAAGCTGGAAAGGATCCTTGCCCA | |
| | GGCCCTGAGAAGGTGGCGGCAGGGCAGGGCTCAACCACTGAGACTCAG | |
| | TCAGTGCCTGGCTTCCAGCAAGCATTCATCTATCACTGTGTCTGCGAGA | |
| | GAGGACTGGCCTTGCAGGGCGCAGGGCCCTAAGCTGGGCTGCAGAGCT | |
| | GGTGGTGAGCTCCTTGCCTGGGTGTGTGTGCGTGTGTGTGTGTGTTCTGT | |
| | GCACTGGGTGTGTGACCTAGGAGGTCCAGGCAGCATGTGTGGTATAAG | |
| | CATTATGAGGGTGATATGCCCCGGTGCAGCATGACCCTGTATGTGGCAC | |
| | CAACAGCATGTGCCTTGTGTGTGTGTGTGTCCGTATGTGTGTGTGTGTAT | |
| | GCGTGTGTGTGTGTGTGTGTGTGTGTCTTGGCCACTGTCATGTGCACTAA | |
| | ATGCTGTGTGTGTGACATGCCCCAAGAGTGTGGCATTTGCCCTGGGTGT | |
| | GGCATCCGCAGCATGTGGCTGTGTGGGTGTCAAGGAGTGGTGGCTCCTT | |
| | CAGCATGCGTTGCGAAGTGCTTGTGCCCTGCATGTGCGGTGTGTTCTCT | |
| | GTACACAGGAGGCTGCCTCAGATGGGGCTGCGGGGTCTGCTGACCTCTG | |
| | CCCTCTGCCCACAGAGCCCTGCCTGGCTCCCAGCCTGGAGCAGCAAGAG | |
| | CAGGAGCTGACAGTAGCCCTTGGGCAGCCTGTGCGGCTGTGCTGTGGGC | |
| | GGGCTGAGCGTGGTGGCCACTGGTACAAGGAGGGCAGTCGCCTGGCAC | |
| | CTGCTGGCCGTGTACGGGGCTGGAGGGGCCGCCTAGAGATTGCCAGCTT | |
| | CCTACCTGAGGATGCTGGCCGCTACCTCTGCCTGGCACGAGGCTCCATG | |
| | ATCGTCCTGCAGAATCTCACCTTGATTACAGGTGACTCCTTGACCTCCA | |
| | GCAACGATGATGAGGACCCCAAGTCCCATAGGGACCTCTCGAATAGGC | |
| | ACAGTTACCCCCAGCAAGGTCAGTAGGTCTCCAAGGACTTGTGTCCCCG | |
| | CTGCTGCTCATCTGATCACTGAGAAGAGGAGGCCTGTGTGGGAACACA | |
| | CGGTCATTCTAGGGGCCTTCCCCTGCCCTCCAGCACCCTACTGGACACA | |
| | CCCCCAGCGCATGGAGAAGAAACTGCATGCAGTACCTGCGGGGAACAC | |
| | CGTCAAGTTCCGCTGTCCAGCTGCAGGCAACCCCACGCCCACCATCCGC | |
| | TGGCTTAAGGATGGACAGGCCTTTCATGGGGAGAACCGCATTGGAGGC | |
| | ATTCGGCTGCGCCATCAGCACTGGAGTCTCGTGATGGAGAGCGTGGTGC | |
| | CCTCGGACCGCGGCACATACACCTGCCTGGTAGAGAACGCTGTGGGCA | |
| | GCATCCGTTATAACTACCTGCTAGATGTGCTGGAGCGGTCCCCGCACCG | |
| | GCCCATCCTGCAGGCCGGGCTCCCGGCCAACACCACAGCCGTGGTGGG | |
| | CAGCGACGTGGAGCTGCTGTGCAAGGTGTACAGCGATGCCCAGCCCCA | |
| | CATCCAGTGGCTGAAGCACATCGTCATCAACGGCAGCAGCTTCGGAGC | |
| | CGACGGTTTCCCCTATGTGCAAGTCCTAAAGACTGCAGACATCAATAGC | |
| | TCAGAGGTGGAGGTCCTGTACCTGCGGAACGTGTCAGCCGAGGACGCA | |
| | GGCGAGTACACCTGCCTCGCAGGCAATTCCATCGGCCTCTCCTACCAGT | |
| | CTGCCTGGCTCACGGTGCTGCCAGGTGAGCACCTGAAGGGCCAGGAGA | |
| | TGCTGCGAGATGCCCCTCTGGGCCAGCAGTGGGGGCTGTGGCCTGTTGG | |
| | GTGGTCAGTCTCTGTTGGCCTGTGGGGTCTGGCCTGGGGGGCAGTGTGT | |
| | GGATTTGTGGGTTTGAGCTGTATGACAGCCCCTCTGTGCCTCTCCACAC | |
| | GTGGCCGTCCATGTGACCGTCTGCTGAGGTGTGGGTGCCTGGGACTGGG | |
| | CATAACTACAGCTTCCTCCGTGTGTGTCCCCACATATGTTGGGAGCTGG | |
| | GAGGGACTGAGTTAGGGTGCACGGGGCGGCCAGTCTCACCACTGACCA | |
| | GTTTGTCTGTCTGTGTGTGTCCATGTGCGAGGGCAGAGGAGGACCCCAC | |
| | ATGGACCGCAGCAGCGCCCGAGGCCAGGTATACGGACATCATCCTGTA | |
| | CGCGTCGGGCTCCCTGGCCTTGGCTGTGCTCCTGCTGCTGGCCAGGCTG | |
| | TATCGAGGGCAGGCGCTCCACGGCCGGCACCCCCGCCCGCCCGCCACT | |
| | GTGCAGAAGCTCTCCCGCTTCCCTCTGGCCCGACAGTTCTCCCTGGAGT | |
| | CAGGCTCTTCCGGCAAGTCAAGCTCATCCCTGGTACGAGGCGTGCGTCT | |
| | CTCCTCCAGCGGCCCCGCCTTGCTCGCCGGCCTCGTGAGTCTAGATCTA | |
| | CCTCTCGACCCACTATGGGAGTTCCCCCGGGACAGGCTGGTGCTTGGGA | |
| | AGCCCCTAGGCGAGGGCTGCTTTGGCCAGGTAGTACGTGCAGAGGCCTT | |
| | TGGCATGGACCCTGCCCGGCCTGACCAAGCCAGCACTGTGGCCGTCAA | |
| | GATGCTCAAAGACAACGCCTCTGACAAGGACCTGGCCGACCTGGTCTC | |
| | GGAGATGGAGGTGATGAAGCTGATCGGCCGACACAAGAACATCATCAA | |
| | CCTGCTTGGTGTCTGCACCCAGGAAGGGCCCCTGTACGTGATCGTGGAG | |
| | TGCGCCGCCAAGGGAAACCTGCGGGAGTTCCTGCGGGCCCGGCGCCCC | |
| | CCAGGCCCCGACCTCAGCCCCGACGGTCCTCGGAGCAGTGAGGGGCCG | |
| | CTCTCCTTCCCAGTCCTGGTCTCCTGCGCCTACCAGGTGGCCCGAGGCA | |
| | TGCAGTATCTGGAGTCCCGGAAGTGTATCCACCGGGACCTGGCTGCCCG | |
| | CAATGTGCTGGTGACTGAGGACAATGTGATGAAGATTGCTGACTTTGGG | |
| | CTGGCCCGCGGCGTCCACCACATTGACTACTATAAGAAAACCAGCAAC | |
| | GGCCGCCTGCCTGTGAAGTGGATGGCGCCCGAGGCCTTGTTTGACCGGG | |
| | TGTACACACACCAGAGTGACGTGTGGTCTTTTGGGATCCTGCTATGGGA | |
| | GATCTTCACCCTCGGGGGCTCCCCGTATCCTGGCATCCCGGTGGAGGAG | |
| | CTGTTCTCGCTGCTGCGGGAGGGACATCGGATGGACCGACCCCCACACT | |
| | GCCCCCCAGAGCTGTACGGGCTGATGCGTGAGTGCTGGCACGCAGCGC | |
| | CCTCCCAGAGGCCTACCTTCAAGCAGCTGGTGGAGGCGCTGGACAAGG | |
| | TCCTGCTGGCCGTCTCTGAGGAGTACCTCGACCTCCGCCTGACCTTCGG | |
| | ACCCTATTCCCCCTCTGGTGGGGACGCCAGCAGCACCTGCTCCTCCAGC | |
| | GATTCTGTCTTCAGCCACGACCCCCTGCCATTGGGATCCAGCTCCTTCCC | |
| | CTTCGGGTCTGGGGTGCAGACATGAGCAAGGCTCAAGGCTGTGCAGGC | |
| | ACATAGGCTGGTGGCCTTGGGCCTTGGGGCTCAGCCACAGCCTGACACA | |
| | GTGCTCGACCTTGATAGCATGGGGCCCCTGGCCCAGAGTTGCTGTGCCG | |
| | TGTCCAAGGGCCGTGCCCTTGCCCTTGGAGCTGCCGTGCCTGTGTCCTG | |
| | ATGGCCCAAATGTCAGGGTTCTGCTCGGCTTCTTGGACCTTGGCGCTTA | |
| | GTCCCCATCCCGGGTTTGGCTGAGCCTGGCTGGAGAGCTGCTATGCTAA | |
| | ACCTCCTGCCTCCCAATACCAGCAGGAGGTTCTGGGCCTCTGAACCCCC | |
| | TTTCCCCACACCTCCCCCTGCTGCTGCTGCCCCAGCGTCTTGACGGGAG | |
| | CATTGGCCCCTGAGCCCAGAGAAGCTGGAAGCCTGCCGAAAACAGGAG | |
| | CAAATGGCGTTTTATAAATTATTTTTTTGAAAT | |
| NM_004496 | TAAGATCCACATCAGCTCAACTGCACTTGCCTCGCAGAGGCAGCCCGCT | 168 |
| | CACTTCCCGCGGAGGCGCTCCCCGGCGCCGCGCTCCGCGGCAGCCGCCT | |
| | GCCCCCGGCGCTGCCCCCGCCCGCCGCGCCGCCGCCGCCGCCGCGCACG | |
| | CCGCGCCCCGCAGCTCTGGGCTTCCTCTTCGCCCGGGTGGCGTTGGGCC | |
| | CGCGCGGGCGCTCGGGTGACTGCAGCTGCTCAGCTCCCCTCCCCCGCCC | |
| | CGCGCCGCGCGGCCGCCCGTCGCTTCGCACAGGGCTGGATGGTTGTATT | |
| | GGGCAGGGTGGCTCCAGGATGTTAGGAACTGTGAAGATGGAAGGGCAT | |
| | GAAACCAGCGACTGGAACAGCTACTACGCAGACACGCAGGAGGCCTAC | |
| | TCCTCCGTCCCGGTCAGCAACATGAACTCAGGCCTGGGCTCCATGAACT | |
| | CCATGAACACCTACATGACCATGAACACCATGACTACGAGCGGCAACA | |
| | TGACCCCGGCGTCCTTCAACATGTCCTATGCCAACCCGGGCCTAGGGGC | |
| | CGGCCTGAGTCCCGGCGCAGTAGCCGGCATGCCGGGGGGCTCGGCGGG | |
| | CGCCATGAACAGCATGACTGCGGCCGGCGTGACGGCCATGGGTACGGC | |
| | GCTGAGCCCGAGCGGCATGGGCGCCATGGGTGCGCAGCAGGCGGCCTC | |
| | CATGAATGGCCTGGGCCCCTACGCGGCCGCCATGAACCCGTGCATGAG | |
| | CCCCATGGCGTACGCGCCGTCCAACCTGGGCCGCAGCCGCGCGGGCGG | |
| | CGGCGGCGACGCCAAGACGTTCAAGCGCAGCTACCCGCACGCCAAGCC | |
| | GCCCTACTCGTACATCTCGCTCATCACCATGGCCATCCAGCAGGCGCCC | |
| | AGCAAGATGCTCACGCTGAGCGAGATCTACCAGTGGATCATGGACCTCT | |
| | TCCCCTATTACCGGCAGAACCAGCAGCGCTGGCAGAACTCCATCCGCCA | |
| | CTCGCTGTCCTTCAATGACTGCTTCGTCAAGGTGGCACGCTCCCCGGAC | |
| | AAGCCGGGCAAGGGCTCCTACTGGACGCTGCACCCGGACTCCGGCAAC | |
| | ATGTTCGAGAACGGCTGCTACTTGCGCCGCCAGAAGCGCTTCAAGTGCG | |
| | AGAAGCAGCCGGGGGCCGGCGGCGGGGGCGGGAGCGGAAGCGGGGGC | |
| | AGCGGCGCCAAGGGCGGCCCTGAGAGCCGCAAGGACCCCTCTGGCGCC | |
| | TCTAACCCCAGCGCCGACTCGCCCCTCCATCGGGGTGTGCACGGGAAGA | |
| | CCGGCCAGCTAGAGGGCGCGCCGGCCCCCGGGCCCGCCGCCAGCCCCC | |
| | AGACTCTGGACCACAGTGGGGCGACGGCGACAGGGGGCGCCTCGGAGT | |
| | TGAAGACTCCAGCCTCCTCAACTGCGCCCCCCATAAGCTCCGGGCCCGG | |
| | GGCGCTGGCCTCTGTGCCCGCCTCTCACCCGGCACACGGCTTGGCACCC | |
| | CACGAGTCCCAGCTGCACCTGAAAGGGGACCCCCACTACTCCTTCAACC | |
| | ACCCGTTCTCCATCAACAACCTCATGTCCTCCTCGGAGCAGCAGCATAA | |
| | GCTGGACTTCAAGGCATACGAACAGGCACTGCAATACTCGCCTTACGGC | |
| | TCTACGTTGCCCGCCAGCCTGCCTCTAGGCAGCGCCTCGGTGACCACCA | |
| | GGAGCCCCATCGAGCCCTCAGCCCTGGAGCCGGCGTACTACCAAGGTG | |
| | TGTATTCCAGACCCGTCCTAAACACTTCCTAGCTCCCGGGACTGGGGGG | |
| | TTTGTCTGGCATAGCCATGCTGGTAGCAAGAGAGAAAAAATCAACAGC | |
| | AAACAAAACCACACAAACCAAACCGTCAACAGCATAATAAAATCCCAA | |
| | CAACTATTTTTATTTCATTTTTCATGCACAACCTTTCCCCCAGTGCAAAA | |
| | GACTGTTACTTTATTATTGTATTCAAAATTCATTGTGTATATTACTACAA | |
| | AGACAACCCCAAACCAATTTTTTTCCTGCGAAGTTTAATGATCCACAAG | |
| | TGTATATATGAAATTCTCCTCCTTCCTTGCCCCCCTCTCTTTCTTCCCTCT | |
| | TTCCCCTCCAGACATTCTAGTTTGTGGAGGGTTATTTAAAAAAACAAAA | |
| | AAGGAAGATGGTCAAGTTTGTAAAATATTTGTTTGTGCTTTTTCCCCCTC | |
| | CTTACCTGACCCCCTACGAGTTTACAGGTCTGTGGCAATACTCTTAACC | |
| | ATAAGAATTGAAATGGTGAAGAAACAAGTATACACTAGAGGCTCTTAA | |
| | AAGTATTGAAAGACAATACTGCTGTTATATAGCAAGACATAAACAGAT | |
| | TATAAACATCAGAGCCATTTGCTTCTCAGTTTACATTTCTGATACATGCA | |
| | GATAGCAGATGTCTTTAAATGAAATACATGTATATTGTGTATGGACTTA | |
| | ATTATGCACATGCTCAGATGTGTAGACATCCTCCGTATATTTACATAAC | |
| | ATATAGAGGTAATAGATAGGTGATATACATGATACATTCTCAAGAGTTG | |
| | CTTGACCGAAAGTTACAAGGACCCCAACCCCTTTGTCCTCTCTACCCAC | |
| | AGATGGCCCTGGGAATCAATTCCTCAGGAATTGCCCTCAAGAACTCTGC | |
| | TTCTTGCTTTGCAGAGTGCCATGGTCATGTCATTCTGAGGTCACATAAC | |
| | ACATAAAATTAGTTTCTATGAGTGTATACCATTTAAAGAATTTTTTTTTC | |
| | AGTAAAAGGGAATATTACAATGTTGGAGGAGAGATAAGTTATAGGGAG | |
| | CTGGATTTCAAAACGTGGTCCAAGATTCAAAAATCCTATTGATAGTGGC | |
| | CATTTTAATCATTGCCATCGTGTGCTTGTTTCATCCAGTGTTATGCACTT | |
| | TCCACAGTTGGACATGGTGTTAGTATAGCCAGACGGGTTTCATTATTAT | |
| | TTCTCTTTGCTTTCTCAATGTTAATTTATTGCATGGTTTATTCTTTTTCTTT | |
| | ACAGCTGAAATTGCTTTAAATGATGGTTAAAATTACAAATTAAATTGTT | |
| | AATTTTTATCAATGTGATTGTAATTAAAAATATTTTGATTTAAATAACAA | |
| | AAATAATACCAGATTTTAAGCCGTGGAAAATGTTCTTGATCATTTGCAG | |
| | TTAAGGACTTTAAATAAATCAAATGTTAACAAAAAAAAAAAAAAAA | |
| NM_001453 | ATGCAGGCGCGCTACTCCGTGTCCAGCCCCAACTCCCTGGGAGTGGTGC | 169 |
| | CCTACCTCGGCGGCGAGCAGAGCTACTACCGCGCGGCGGCCGCGGCGG | |
| | CCGGGGGCGGCTACACCGCCATGCCGGCCCCCATGAGCGTGTACTCGC | |
| | ACCCTGCGCACGCCGAGCAGTACCCGGGCGGCATGGCCCGCGCCTACG | |
| | GGCCCTACACGCCGCAGCCGCAGCCCAAGGACATGGTGAAGCCGCCCT | |
| | ATAGCTACATCGCGCTCATCACCATGGCCATCCAGAACGCCCCGGACAA | |
| | GAAGATCACCCTGAACGGCATCTACCAGTTCATCATGGACCGCTTCCCC | |
| | TTCTACCGGGACAACAAGCAGGGCTGGCAGAACAGCATCCGCCACAAC | |
| | CTCTCGCTCAACGAGTGCTTCGTCAAGGTGCCGCGCGACGACAAGAAG | |
| | CCGGGCAAGGGCAGCTACTGGACGCTGGACCCGGACTCCTACAACATG | |
| | TTCGAGAACGGCAGCTTCCTGCGGCGGCGGCGGCGCTTCAAGAAGAAG | |
| | GACGCGGTGAAGGACAAGGAGGAGAAGGACAGGCTGCACCTCAAGGA | |
| | GCCGCCCCCGCCCGGCCGCCAGCCCCCGCCCGCGCCGCCGGAGCAGGC | |
| | CGACGGCAACGCGCCCGGTCCGCAGCCGCCGCCCGTGCGCATCCAGGA | |
| | CATCAAGACCGAGAACGGTACGTGCCCCTCGCCGCCCCAGCCCCTGTCC | |
| | CCGGCCGCCGCCCTGGGCAGCGGCAGCGCCGCCGCGGTGCCCAAGATC | |
| | GAGAGCCCCGACAGCAGCAGCAGCAGCCTGTCCAGCGGGAGCAGCCCC | |
| | CCGGGCAGCCTGCCGTCGGCGCGGCCGCTCAGCCTGGACGGTGCGGAT | |
| | TCCGCGCCGCCGCCGCCCGCGCCCTCCGCCCCGCCGCCGCACCATAGCC | |
| | AGGGCTTCAGCGTGGACAACATCATGACGTCGCTGCGGGGGTCGCCGC | |
| | AGAGCGCGGCCGCGGAGCTCAGCTCCGGCCTTCTGGCCTCGGCGGCCG | |
| | CGTCCTCGCGCGCGGGGATCGCACCCCCGCTGGCGCTCGGCGCCTACTC | |
| | GCCCGGCCAGAGCTCCCTCTACAGCTCCCCCTGCAGCCAGACCTCCAGC | |
| | GCGGGCAGCTCGGGCGGCGGCGGCGGCGGCGCGGGGGCCGCGGGGGG | |
| | CGCGGGCGGCGCCGGGACCTACCACTGCAACCTGCAAGCCATGAGCCT | |
| | GTACGCGGCCGGCGAGCGCGGGGGCCACTTGCAGGGCGCGCCCGGGGG | |
| | CGCGGGCGGCTCGGCCGTGGACGACCCCCTGCCCGACTACTCTCTGCCT | |
| | CCGGTCACCAGCAGCAGCTCGTCGTCCCTGAGTCACGGCGGCGGCGGC | |
| | GGCGGCGGCGGGGGAGGCCAGGAGGCCGGCCACCACCCTGCGGCCCAC | |
| | CAAGGCCGCCTCACCTCGTGGTACCTGAACCAGGCGGGCGGAGACCTG | |
| | GGCCACTTGGCGAGCGCGGCGGCGGCGGCGGCGGCCGCAGGCTACCCG | |
| | GGCCAGCAGCAGAACTTCCACTCGGTGCGGGAGATGTTCGAGTCACAG | |
| | AGGATCGGCTTGAACAACTCTCCAGTGAACGGGAATAGTAGCTGTCAA | |
| | ATGGCCTTCCCTTCCAGCCAGTCTCTGTACCGCACGTCCGGAGCTTTCGT | |
| | CTACGACTGTAGCAAGTTTTGACACACCCTCAAAGCCGAACTAAATCGA | |
| | ACCCCAAAGCAGGAAAAGCTAAAGGAACCCATCAAGGCAAAATCGAA | |
| | ACTAAAAAAAAAAAATCCATTAAAAAAAACCCCTGAGAATATTCACC | |
| | ACACCAGCGAACAGAATATCCCTCCAAAAATTCAGCTCACCAGCACCA | |
| | GCACGAAGAAAACTCTATTTTCTTAACCGATTAATTCAGAGCCACCTCC | |
| | ACTTTGCCTTGTCTAAATAAACAAACCCGTAAACTGTTTTATACAGAGA | |
| | CAGCAAAATCTTGGTTTATTAAAGGACAGTGTTACTCCAGATAACACGT | |
| | AAGTTTCTTCTTGCTTTTCAGAGACCTGCTTTCCCCTCCTCCCGTCTCCCC | |
| | TCTCTTGCCTTCTTCCTTGCCTCTCACCTGTAAGATATTATTTTATCCTAT | |
| | GTTGAAGGGAGGGGGAAAGTCCCCGTTTATGAAAGTCGCTTTCTTTTTA | |
| | TTCATGGACTTGTTTTAAAATGTAAATTGCAACATAGTAATTTATTTTTA | |
| | ATTTGTAGTTGGATGTCGTGGACCAAACGCCAGAAAGTGTTCCCAAAAC | |
| | CTGACGTTAAATTGCCTGAAACTTTAAATTGTGCTTTTTTTCTCATTATA | |
| | AAAAGGGAAACTGTATTAATCTTATTCTATCCTCTTTTCTTTCTTTTTGTT | |
| | GAACATATTCATTGTTTGTTTATTAATAAATTACCATTCAGTTTGAATGA | |
| | GACCTATATGTCTGGATACTTTAATAGAGCTTTAATTATTACGAAAAAA | |
| | GATTTCAGAGATAAAACACTAGAAGTTACCTATTCTCCACCTAAATCTC | |
| | TGAAAAATGGAGAAACCCTCTGACTAGTCCATGTCAAATTTTACTAAAA | |
| | GTCTTTTTGTTTAGATTTATTTTCCTGCAGCATCTTCTGCAAAATGTACT | |
| | ATATAGTCAGCTTGCTTTGAGGCTAGTAAAAAGATATTTTTCTAAACAG | |
| | ATTGGAGTTGGCATATAAACAAATACGTTTTCTCACTAATGACAGTCCA | |
| | TGATTCGGAAATTTTAAGCCCATGAATCAGCCGCGGTCTTACCACGGTG | |
| | ATGCCTGTGTGCCGAGAGATGGGACTGTGCGGCCAGATATGCACAGAT | |
| | AAATATTTGGCTTGTGTATTCCATATAAAATTGCAGTGCATATTATACAT | |
| | CCCTGTGAGCCAGATGCTGAATAGATATTTTCCTATTATTTCAGTCCTTT | |
| | ATAAAAGGAAAAATAAACCAGTTTTTAAATGTATGTATATAATTCTCCC | |
| | CCATTTACAATCCTTCATGTATTACATAGAAGGATTGCTTTTTTAAAAAT | |
| | ATACTGCGGGTTGGAAAGGGATATTTAATCTTTGAGAAACTATTTTAGA | |
| | AAATATGTTTGTAGAACAATTATTTTTGAAAAAGATTTAAAGCAATAAC | |
| | AAGAAGGAAGGCGAGAGGAGCAGAACATTTTGGTCTAGGGTGGTTTCT | |
| | TTTTAAACCATTTTTTCTTGTTAATTTACAGTTAAACCTAGGGGACAATC | |
| | CGGATTGGCCCTCCCCCTTTTGTAAATAACCCAGGAAATGTAATAAATT | |
| | CATTATCTTAGGGTGATCTGCCCTGCCAATCAGACTTTGGGGAGATGGC | |
| | GATTTGATTACAGACGTTCGGGGGGGTGGGGGGCTTGCAGTTTGTTTTG | |
| | GAGATAATACAGTTTCCTGCTATCTGCCGCTCCTATCTAGAGGCAACAC | |
| | TTAAGCAGTAATTGCTGTTGCTTGTTGTCAAAATTTGATCATTGTTAAAG | |
| | GATTGCTGCAAATAAATACACTTTAATTTCAGTCAAAAA | |
| AJ249248 | GTGGCCTCGAGGTGGTGGCAGGGCCGCCCCCTGCAGTCCGGAGACGAA | 170 |
| | CGCACGGACCGGGCCTCCGGAGGCAGGTTCGGCTGGAAGGAACCGCTC | |
| | TCGCTTCGTCCTACACTTGCGCAAATGTCTCCGAGCTTACTCACATAGC | |
| | ATATTGGTATATCAAAATGAAATGCAAGGAACCAAAAATAACATAATT | |
| | GAAGGCAGTAAAAGTGAAATTAAATAGGAAGATCATCAGTCAAGGAAG | |
| | ACCCACTGGAGAGGACAGAAAATGAAGCAGTGTTTTATCATGTGTATTT | |
| | CAGCAGGTCTTCTTGAAATTTAACTAAAAATATGACTGCTCTCTCTTCA | |
| | GAGAACTGCTCTTTTCAGTACCAGTTACGTCAAACAAACCAGCCCCTAG | |
| | ACGTTAACTATCTGCTATTCTTGATCATACTTGGGAAAATATTATTAAAT | |
| | ATCCTTACACTAGGAATGAGAAGAAAAAACACCTGTCAAAATTTTATG | |
| | GAATATTTTTGCATTTCACTAGCATTCGTTGATCTTTTACTTTTGGTAAA | |
| | CATTTCCATTATATTGTATTTCAGGGATTTTGTACTTTTAAGCATTAGGT | |
| | TCACTAAATACCACATCTGCCTATTTACTCAAATTATTTCCTTTACTTAT | |
| | GGCTTTTTGCATTATCCAGTTTTCCTGACAGCTTGTATAGATTATTGCCT | |
| | GAATTTCTCTAAAACAACCAAGCTTTCATTTAAGTGTCAAAAATTATTTT | |
| | ATTTCTTTACAGTAATTTTAATTTGGATTTCAGTCCTTGCTTATGTTTTGG | |
| | GAGACCCAGCCATCTACCAAAGCCTGAAGGCACAGAATGCTTATTCTCG | |
| | TCACTGTCCTTTCTATGTCAGCATTCAGAGTTACTGGCTGTCATTTTTCA | |
| | TGGTGATGATTTTATTTGTAGCTTTCATAACCTGTTGGGAAGAAGTTACT | |
| | ACTTTGGTACAGGCTATCAGGATAACTTCCTATATGAATGAAACTATCT | |
| | TATATTTTCCTTTTTCATCCCACTCCAGTTATACTGTGAGATCTAAAAAA | |
| | ATATTCTTATCCAAGCTCATTGTCTGTTTTCTCAGTACCTGGTTACCATT | |
| | TGTACTACTTCAGGTAATCATTGTTTTACTTAAAGTTCAGATTCCAGCAT | |
| | ATATTGAGATGAATATTCCCTGGTTATACTTTGTCAATAGTTTTCTCATT | |
| | GCTACAGTGTATTGGTTTAATTGTCACAAGCTTAATTTAAAAGACATTG | |
| | GATTACCTTTGGATCCATTTGTCAACTGGAAGTGCTGCTTCATTCCACTT | |
| | ACAATTCCTAATCTTGAGCAAATTGAAAAGCCTATATCAATAATGATTT | |
| | GTTAATATTATTAATTAAAAGTTACAGCTGTCATAAGATCATAATTTTAT | |
| | GAACAGAAAGAACTCAGGACATATTAAAAAATAAACTGAACTAAAACA | |
| | ACTTTTGCCCCCTGACTGATAGCATTTCAGAATGTGTCTTTTGAAGGGCT | |
| | ATACCAGTTATTAAATAGTGTTTTATTTTAAAAACAAAATAATTCCAAG | |
| | AAGTTTTTATAGTTATTCAGGGACACTATATTACAAATATTACTTTGTTA | |
| | TTAACACAAAAAGTGATAAGAGTTAACATTTGGCTATACTGATGTTTGT | |
| | GTTACTCAAAAAAACTACTGGATGCAAACTGTTATGTAAATCTGAGATT | |
| | TCACTGACAACTTTAAGATATCAACCTAAACATTTTTATTAAATGTTCA | |
| | AATGTAAGCAAGAAAAAAAAAA | |
| NM_005310 | ACCCGCCCCCATCTGCCCAAGATAATTTTAGTTTCCTTGGGCCTGGAAT | 171 |
| | CTGGACACACAGGGCTCCCCCCCGCCTCTGACTTCTCTGTCCGAAGTCG | |
| | GGACACCCTCCTACCACCTGTAGAGAAGCGGGAGTGGATCTGAAATAA | |
| | AATCCAGGAATCTGGGGGTTCCTAGACGGAGCCAGACTTCGGAACGGG | |
| | TGTCCTGCTACTCCTGCTGGGGCTCCTCCAGGACAAGGGCACACAACTG | |
| | GTTCCGTTAAGCCCCTCTCTCGCTCAGACGCCATGGAGCTGGATCTGTC | |
| | TCCACCTCATCTTAGCAGCTCTCCGGAAGACCTTTGCCCAGCCCCTGGG | |
| | ACCCCTCCTGGGACTCCCCGGCCCCCTGATACCCCTCTGCCTGAGGAGG | |
| | TAAAGAGGTCCCAGCCTCTCCTCATCCCAACCACCGGCAGGAAACTTCG | |
| | AGAGGAGGAGAGGCGTGCCACCTCCCTCCCCTCTATCCCCAACCCCTTC | |
| | CCTGAGCTCTGCAGTCCTCCCTCACAGAGCCCAATTCTCGGGGGCCCCT | |
| | CCAGTGCAAGGGGGCTGCTCCCCCGCGATGCCAGCCGCCCCCATGTAGT | |
| | AAAGGTGTACAGTGAGGATGGGGCCTGCAGGTCTGTGGAGGTGGCAGC | |
| | AGGTGCCACAGCTCGCCACGTGTGTGAAATGCTGGTGCAGCGAGCTCA | |
| | CGCCTTGAGCGACGAGACCTGGGGGCTGGTGGAGTGCCACCCCCACCT | |
| | AGCACTGGAGCGGGGTTTGGAGGACCACGAGTCCGTGGTGGAAGTGCA | |
| | GGCTGCCTGGCCCGTGGGCGGAGATAGCCGCTTCGTCTTCCGGAAAAAC | |
| | TTCGCCAAGTACGAACTGTTCAAGAGCTCCCCACACTCCCTGTTCCCAG | |
| | AAAAAATGGTCTCCAGCTGTCTCGATGCACACACTGGTATATCCCATGA | |
| | AGACCTCATCCAGAACTTCCTGAATGCTGGCAGCTTTCCTGAGATCCAG | |
| | GGCTTTCTGCAGCTGCGGGGTTCAGGACGGAAGCTTTGGAAACGCTTTT | |
| | TCTGCTTCTTGCGCCGATCTGGCCTCTATTACTCCACCAAGGGCACCTCT | |
| | AAGGATCCGAGGCACCTGCAGTACGTGGCAGATGTGAACGAGTCCAAC | |
| | GTGTACGTGGTGACGCAGGGCCGCAAGCTCTACGGGATGCCCACTGAC | |
| | TTCGGTTTCTGTGTCAAGCCCAACAAGCTTCGAAATGGCCACAAGGGGC | |
| | TTCGGATCTTCTGCAGTGAAGATGAGCAGAGCCGCACCTGCTGGCTGGC | |
| | TGCCTTCCGCCTCTTCAAGTACGGGGTGCAGCTGTACAAGAATTACCAG | |
| | CAGGCACAGTCTCGCCATCTGCATCCATCTTGTTTGGGCTCCCCACCCTT | |
| | GAGAAGTGCCTCAGATAATACCCTGGTGGCCATGGACTTCTCTGGCCAT | |
| | GCTGGGCGTGTCATTGAGAACCCCCGGGAGGCTCTGAGTGTGGCCCTGG | |
| | AGGAGGCCCAGGCCTGGAGGAAGAAGACAAACCACCGCCTCAGCCTGC | |
| | CCATGCCAGCCTCCGGCACGAGCCTCAGTGCAGCCATCCACCGCACCCA | |
| | ACTCTGGTTCCACGGGCGCATTTCCCGTGAGGAGAGCCAGCGGCTTATT | |
| | GGACAGCAGGGCTTGGTAGACGGCCTGTTCCTGGTCCGGGAGAGTCAG | |
| | CGGAACCCCCAGGGCTTTGTCCTCTCTTTGTGCCACCTGCAGAAAGTGA | |
| | AGCATTATCTCATCCTGCCGAGCGAGGAGGAGGGCCGCCTGTACTTCAG | |
| | CATGGATGATGGCCAGACCCGCTTCACTGACCTGCTGCAGCTCGTGGAG | |
| | TTCCACCAGCTGAACCGCGGCATCCTGCCGTGCTTGCTGCGCCATTGCT | |
| | GCACGCGGGTGGCCCTCTGACCAGGCCGTGGACTGGCTCATGCCTCAGC | |
| | CCGCCTTCAGGCTGCCCGCCGCCCCTCCACCCATCCAGTGGACTCTGGG | |
| | GCGCGGCCACAGGGGACGGGATGAGGAGCGGGAGGGTTCCGCCACTCC | |
| | AGTTTTCTCCTCTGCTTCTTTGCCTCCCTCAGATAGAAAACAGCCCCCAC | |
| | TCCAGTCCACTCCTGACCCCTCTCCTCAAGGGAAGGCCTTGGGTGGCCC | |
| | CCTCTCCTTCTCCTAGCTCTGGAGGTGCTGCTCTAGGGCAGGGAATTAT | |
| | GGGAGAAGTGGGGGCAGCCCAGGCGGTTTCACGCCCCACACTTTGTAC | |
| | AGACCGAGAGGCCAGTTGATCTGCTCTGTTTTATACTAGTGACAATAAA | |
| | GATTATTTTTTGATACAAAAAAAAAAAAAAAAAAAAAAAA | |
| NM_014176 | AGTCAGAGGTCGCGCAGGCGCTGGTACCCCGTTGGTCCGCGCGTTGCTG | 172 |
| | CGTTGTGAGGGGTGTCAGCTCAGTGCATCCCAGGCAGCTCTTAGTGTGG | |
| | AGCAGTGAACTGTGTGTGGTTCCTTCTACTTGGGGATCATGCAGAGAGC | |
| | TTCACGTCTGAAGAGAGAGCTGCACATGTTAGCCACAGAGCCACCCCC | |
| | AGGCATCACATGTTGGCAAGATAAAGACCAAATGGATGACCTGCGAGC | |
| | TCAAATATTAGGTGGAGCCAACACACCTTATGAGAAAGGTGTTTTTAAG | |
| | CTAGAAGTTATCATTCCTGAGAGGTACCCATTTGAACCTCCTCAGATCC | |
| | GATTTCTCACTCCAATTTATCATCCAAACATTGATTCTGCTGGAAGGATT | |
| | TGTCTGGATGTTCTCAAATTGCCACCAAAAGGTGCTTGGAGACCATCCC | |
| | TCAACATCGCAACTGTGTTGACCTCTATTCAGCTGCTCATGTCAGAACC | |
| | CAACCCTGATGACCCGCTCATGGCTGACATATCCTCAGAATTTAAATAT | |
| | AATAAGCCAGCCTTCCTCAAGAATGCCAGACAGTGGACAGAGAAGCAT | |
| | GCAAGACAGAAACAAAAGGCTGATGAGGAAGAGATGCTTGATAATCTA | |
| | CCAGAGGCTGGTGACTCCAGAGTACACAACTCAACACAGAAAAGGAAG | |
| | GCCAGTCAGCTAGTAGGCATAGAAAAGAAATTTCATCCTGATGTTTAGG | |
| | GGACTTGTCCTGGTTCATCTTAGTTAATGTGTTCTTTGCCAAGGTGATCT | |
| | AAGTTGCCTACCTTGAATTTTTTTTTAAATATATTTGATGACATAATTTT | |
| | TGTGTAGTTTATTTATCTTGTACATATGTATTTTGAAATCTTTTAAACCT | |
| | GAAAAATAAATAGTCATTTAATGTTGAAAAAAAAAAAAAAAAAAAAA | |
| | AAAAAAA | |
| NM_006845 | ACGCTTGCGCGCGGGATTTAAACTGCGGCGGTTTACGCGGCGTTAAGAC | 173 |
| | TTCGTAGGGTTAGCGAAATTGAGGTTTCTTGGTATTGCGCGTTTCTCTTC | |
| | CTTGCTGACTCTCCGAATGGCCATGGACTCGTCGCTTCAGGCCCGCCTG | |
| | TTTCCCGGTCTCGCTATCAAGATCCAACGCAGTAATGGTTTAATTCACA | |
| | GTGCCAATGTAAGGACTGTGAACTTGGAGAAATCCTGTGTTTCAGTGGA | |
| | ATGGGCAGAAGGAGGTGCCACAAAGGGCAAAGAGATTGATTTTGATGA | |
| | TGTGGCTGCAATAAACCCAGAACTCTTACAGCTTCTTCCCTTACATCCG | |
| | AAGGACAATCTGCCCTTGCAGGAAAATGTAACAATCCAGAAACAAAAA | |
| | CGGAGATCCGTCAACTCCAAAATTCCTGCTCCAAAAGAAAGTCTTCGAA | |
| | GCCGCTCCACTCGCATGTCCACTGTCTCAGAGCTTCGCATCACGGCTCA | |
| | GGAGAATGACATGGAGGTGGAGCTGCCTGCAGCTGCAAACTCCCGCAA | |
| | GCAGTTTTCAGTTCCTCCTGCCCCCACTAGGCCTTCCTGCCCTGCAGTGG | |
| | CTGAAATACCATTGAGGATGGTCAGCGAGGAGATGGAAGAGCAAGTCC | |
| | ATTCCATCCGAGGCAGCTCTTCTGCAAACCCTGTGAACTCAGTTCGGAG | |
| | GAAATCATGTCTTGTGAAGGAAGTGGAAAAAATGAAGAACAAGCGAGA | |
| | AGAGAAGAAGGCCCAGAACTCTGAAATGAGAATGAAGAGAGCTCAGG | |
| | AGTATGACAGTAGTTTTCCAAACTGGGAATTTGCCCGAATGATTAAAGA | |
| | ATTTCGGGCTACTTTGGAATGTCATCCACTTACTATGACTGATCCTATCG | |
| | AAGAGCACAGAATATGTGTCTGTGTTAGGAAACGCCCACTGAATAAGC | |
| | AAGAATTGGCCAAGAAAGAAATTGATGTGATTTCCATTCCTAGCAAGTG | |
| | TCTCCTCTTGGTACATGAACCCAAGTTGAAAGTGGACTTAACAAAGTAT | |
| | CTGGAGAACCAAGCATTCTGCTTTGACTTTGCATTTGATGAAACAGCTT | |
| | CGAATGAAGTTGTCTACAGGTTCACAGCAAGGCCACTGGTACAGACAA | |
| | TCTTTGAAGGTGGAAAAGCAACTTGTTTTGCATATGGCCAGACAGGAAG | |
| | TGGCAAGACACATACTATGGGCGGAGACCTCTCTGGGAAAGCCCAGAA | |
| | TGCATCCAAAGGGATCTATGCCATGGCCTCCCGGGACGTCTTCCTCCTG | |
| | AAGAATCAACCCTGCTACCGGAAGTTGGGCCTGGAAGTCTATGTGACAT | |
| | TCTTCGAGATCTACAATGGGAAGCTGTTTGACCTGCTCAACAAGAAGGC | |
| | CAAGCTGCGCGTGCTGGAGGACGGCAAGCAACAGGTGCAAGTGGTGGG | |
| | GCTGCAGGAGCATCTGGTTAACTCTGCTGATGATGTCATCAAGATGATC | |
| | GACATGGGCAGCGCCTGCAGAACCTCTGGGCAGACATTTGCCAACTCC | |
| | AATTCCTCCCGCTCCCACGCGTGCTTCCAAATTATTCTTCGAGCTAAAG | |
| | GGAGAATGCATGGCAAGTTCTCTTTGGTAGATCTGGCAGGGAATGAGC | |
| | GAGGCGCGGACACTTCCAGTGCTGACCGGCAGACCCGCATGGAGGGCG | |
| | CAGAAATCAACAAGAGTCTCTTAGCCCTGAAGGAGTGCATCAGGGCCC | |
| | TGGGACAGAACAAGGCTCACACCCCGTTCCGTGAGAGCAAGCTGACAC | |
| | AGGTGCTGAGGGACTCCTTCATTGGGGAGAACTCTAGGACTTGCATGAT | |
| | TGCCACGATCTCACCAGGCATAAGCTCCTGTGAATATACTTTAAACACC | |
| | CTGAGATATGCAGACAGGGTCAAGGAGCTGAGCCCCCACAGTGGGCCC | |
| | AGTGGAGAGCAGTTGATTCAAATGGAAACAGAAGAGATGGAAGCCTGC | |
| | TCTAACGGGGCGCTGATTCCAGGCAATTTATCCAAGGAAGAGGAGGAA | |
| | CTGTCTTCCCAGATGTCCAGCTTTAACGAAGCCATGACTCAGATCAGGG | |
| | AGCTGGAGGAGAAGGCTATGGAAGAGCTCAAGGAGATCATACAGCAA | |
| | GGACCAGACTGGCTTGAGCTCTCTGAGATGACCGAGCAGCCAGACTAT | |
| | GACCTGGAGACCTTTGTGAACAAAGCGGAATCTGCTCTGGCCCAGCAA | |
| | GCCAAGCATTTCTCAGCCCTGCGAGATGTCATCAAGGCCTTGCGCCTGG | |
| | CCATGCAGCTGGAAGAGCAGGCTAGCAGACAAATAAGCAGCAAGAAA | |
| | CGGCCCCAGTGACGACTGCAAATAAAAATCTGTTTGGTTTGACACCCAG | |
| | CCTCTTCCCTGGCCCTCCCCAGAGAACTTTGGGTACCTGGTGGGTCTAG | |
| | GCAGGGTCTGAGCTGGGACAGGTTCTGGTAAATGCCAAGTATGGGGGC | |
| | ATCTGGGCCCAGGGCAGCTGGGGAGGGGGTCAGAGTGACATGGGACAC | |
| | TCCTTTTCTGTTCCTCAGTTGTCGCCCTCACGAGAGGAAGGAGCTCTTAG | |
| | TTACCCTTTTGTGTTGCCCTTCTTTCCATCAAGGGGAATGTTCTCAGCAT | |
| | AGAGCTTTCTCCGCAGCATCCTGCCTGCGTGGACTGGCTGCTAATGGAG | |
| | AGCTCCCTGGGGTTGTCCTGGCTCTGGGGAGAGAGACGGAGCCTTTAGT | |
| | ACAGCTATCTGCTGGCTCTAAACCTTCTACGCCTTTGGGCCGAGCACTG | |
| | AATGTCTTGTACTTTAAAAAAATGTTTCTGAGACCTCTTTCTACTTTACT | |
| | GTCTCCCTAGAGATCCTAGAGGATCCCTACTGTTTTCTGTTTTATGTGTT | |
| | TATACATTGTATGTAACAATAAAGAGAAAAAATAAATCAGCTGTTTAA | |
| | GTGTGTGGAAAAAAAAAAAAAAAAAA | |
| NM_006101 | ACTGCGCGCGTCGTGCGTAATGACGTCAGCGCCGGCGGAGAATTTCAA | 174 |
| | ATTCGAACGGCTTTGGCGGGCCGAGGAAGGACCTGGTGTTTTGATGACC | |
| | GCTGTCCTGTCTAGCAGATACTTGCACGGTTTACAGAAATTCGGTCCCT | |
| | GGGTCGTGTCAGGAAACTGGAAAAAAGGTCATAAGCATGAAGCGCAGT | |
| | TCAGTTTCCAGCGGTGGTGCTGGCCGCCTCTCCATGCAGGAGTTAAGAT | |
| | CCCAGGATGTAAATAAACAAGGCCTCTATACCCCTCAAACCAAAGAGA | |
| | AACCAACCTTTGGAAAGTTGAGTATAAACAAACCGACATCTGAAAGAA | |
| | AAGTCTCGCTATTTGGCAAAAGAACTAGTGGACATGGATCCCGGAATA | |
| | GTCAACTTGGTATATTTTCCAGTTCTGAGAAAATCAAGGACCCGAGACC | |
| | ACTTAATGACAAAGCATTCATTCAGCAGTGTATTCGACAACTCTGTGAG | |
| | TTTCTTACAGAAAATGGTTATGCACATAATGTGTCCATGAAATCTCTAC | |
| | AAGCTCCCTCTGTTAAAGACTTCCTGAAGATCTTCACATTTCTTTATGGC | |
| | TTCCTGTGCCCCTCATACGAACTTCCTGACACAAAGTTTGAAGAAGAGG | |
| | TTCCAAGAATCTTTAAAGACCTTGGGTATCCTTTTGCACTATCCAAAAG | |
| | CTCCATGTACACAGTGGGGGCTCCTCATACATGGCCTCACATTGTGGCA | |
| | GCCTTAGTTTGGCTAATAGACTGCATCAAGATACATACTGCCATGAAAG | |
| | AAAGCTCACCTTTATTTGATGATGGGCAGCCTTGGGGAGAAGAAACTG | |
| | AAGATGGAATTATGCATAATAAGTTGTTTTTGGACTACACCATAAAATG | |
| | CTATGAGAGTTTTATGAGTGGTGCCGACAGCTTTGATGAGATGAATGCA | |
| | GAGCTGCAGTCAAAACTGAAGGATTTATTTAATGTGGATGCTTTTAAGC | |
| | TGGAATCATTAGAAGCAAAAAACAGAGCATTGAATGAACAGATTGCAA | |
| | GATTGGAACAAGAAAGAGAAAAAGAACCGAATCGTCTAGAGTCGTTGA | |
| | GAAAACTGAAGGCTTCCTTACAAGGAGATGTTCAAAAGTATCAGGCAT | |
| | ACATGAGCAATTTGGAGTCTCATTCAGCCATTCTTGACCAGAAATTAAA | |
| | TGGTCTCAATGAGGAAATTGCTAGAGTAGAACTAGAATGTGAAACAAT | |
| | AAAACAGGAGAACACTCGACTACAGAATATCATTGACAACCAGAAGTA | |
| | CTCAGTTGCAGACATTGAGCGAATAAATCATGAAAGAAATGAATTGCA | |
| | GCAGACTATTAATAAATTAACCAAGGACCTGGAAGCTGAACAACAGAA | |
| | GTTGTGGAATGAGGAGTTAAAATATGCCAGAGGCAAAGAAGCGATTGA | |
| | AACACAATTAGCAGAGTATCACAAATTGGCTAGAAAATTAAAACTTATT | |
| | CCTAAAGGTGCTGAGAATTCCAAAGGTTATGACTTTGAAATTAAGTTTA | |
| | ATCCCGAGGCTGGTGCCAACTGCCTTGTCAAATACAGGGCTCAAGTTTA | |
| | TGTACCTCTTAAGGAACTCCTGAATGAAACTGAAGAAGAAATTAATAA | |
| | AGCCCTAAATAAAAAAATGGGTTTGGAGGATACTTTAGAACAATTGAA | |
| | TGCAATGATAACAGAAAGCAAGAGAAGTGTGAGAACTCTGAAAGAAG | |
| | AAGTTCAAAAGCTGGATGATCTTTACCAACAAAAAATTAAGGAAGCAG | |
| | AGGAAGAGGATGAAAAATGTGCCAGTGAGCTTGAGTCCTTGGAGAAAC | |
| | ACAAGCACCTGCTAGAAAGTACTGTTAACCAGGGGCTCAGTGAAGCTA | |
| | TGAATGAATTAGATGCTGTTCAGCGGGAATACCAACTAGTTGTGCAAAC | |
| | CACGACTGAAGAAAGACGAAAAGTGGGAAATAACTTGCAACGTCTGTT | |
| | AGAGATGGTTGCTACACATGTTGGGTCTGTAGAGAAACATCTTGAGGA | |
| | GCAGATTGCTAAAGTTGATAGAGAATATGAAGAATGCATGTCAGAAGA | |
| | TCTCTCGGAAAATATTAAAGAGATTAGAGATAAGTATGAGAAGAAAGC | |
| | TACTCTAATTAAGTCTTCTGAAGAATGAAGATAAAATGTTGATCATGTA | |
| | TATATATCCATAGTGAATAAAATTGTCTCAGTAAAGTGTAAAAAAAAA | |
| | AAAAAAAAAAAAAAAAA | |
| BC042437 | CTCCCTCCTCTGCACCATGACTACCTGCAGCCGCCAGTTCACCTCCTCCA | 175 |
| | GCTCCATGAAGGGCTCCTGCGGCATCGGGGGCGGCATCGGGGGCGGCT | |
| | CCAGCCGCATCTCCTCCGTCCTGGCCGGAGGGTCCTGCCGCGCCCCCAG | |
| | CACCTACGGGGGCGGCCTGTCTGTCTCATCCTCCCGCTTCTCCTCTGGGG | |
| | GAGCCTATGGGTTGGGGGGCGGCTATGGCGGTGGCTTCAGCAGCAGCA | |
| | GCAGCAGCTTTGGTAGTGGCTTTGGGGGAGGATATGGTGGTGGCCTTGG | |
| | TGCTGGCTTGGGTGGTGGCTTTGGTGGTGGCTTTGCTGGTGGTGATGGG | |
| | CTTCTGGTGGGCAGTGAGAAGGTGACCATGCAGAACCTCAACGACCGC | |
| | CTGGCCTCCTACCTGGACAAGGTGCGTGCTCTGGAGGAGGCCAACGCC | |
| | GACCTGGAAGTGAAGATCCGTGACTGGTACCAGAGGCAGCGGCCTGCT | |
| | GAGATCAAAGACTACAGTCCCTACTTCAAGACCATTGAGGACCTGAGG | |
| | AACAAGATTCTCACAGCCACAGTGGACAATGCCAATGTCCTTCTGCAGA | |
| | TTGACAATGCCCGTCTGGCCGCGGATGACTTCCGCACCAAGTATGAGAC | |
| | AGAGTTGAACCTGCGCATGAGTGTGGAAGCCGACATCAATGGCCTGCG | |
| | CAGGGTGCTGGACGAACTGACCCTGGCCAGAGCTGACCTGGAGATGCA | |
| | GATTGAGAGCCTGAAGGAGGAGCTGGCCTACCTGAAGAAGAACCACGA | |
| | GGAGGAGATGAATGCCCTGAGAGGCCAGGTGGGTGGAGATGTCAATGT | |
| | GGAGATGGACGCTGCACCTGGCGTGGACCTGAGCCGCATTCTGAACGA | |
| | GATGCGTGACCAGTATGAGAAGATGGCAGAGAAGAACCGCAAGGATGC | |
| | CGAGGAATGGTTCTTCACCAAGACAGAGGAGCTGAACCGCGAGGTGGC | |
| | CACCAACAGCGAGCTGGTGCAGAGCGGCAAGAGCGAGATCTCGGAGCT | |
| | CCGGCGCACCATGCAGAACCTGGAGATTGAGCTGCAGTCCCAGCTCAG | |
| | CATGAAAGCATCCCTGGAGAACAGCCTGGAGGAGACCAAAGGTCGCTA | |
| | CTGCATGCAGCTGGCCCAGATCCAGGAGATGATTGGCAGCGTGGAGGA | |
| | GCAGCTGGCCCAGCTCCGCTGCGAGATGGAGCAGCAGAACCAGGAGTA | |
| | CAAGATCCTGCTGGACGTGAAGACGCGGCTGGAGCAGGAGATCGCCAC | |
| | CTACCGCCGCCTGCTGGAGGGCGAGGACGCCCACCTCTCCTCCTCCCAG | |
| | TTCTCCTCTGGATCGCAGTCATCCAGAGATGTGACCTCCTCCAGCCGCC | |
| | AAATCCGCACCAAGGTCATGGATGTGCACGATGGCAAGGTGGTGTCCA | |
| | CCCACGAGCAGGTCCTTCGCACCAAGAACTGAGGCTGCCCAGCCCCGCT | |
| | CAGGCCTAGGAGGCCCCCCGTGTGGACACAGATCCCACTGGAAGATCC | |
| | CCTCTCCTGCCCAAGCACTTCACAGCTGGACCCTGCTTCACCCTCACCCC | |
| | CTCCTGGCAATCAATACAGCTTCATTATCTGAGTTGCATAAAAAAAAAA | |
| | AAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAA | |
| | AAA | |
| AK095281 | CTCTTTTGCAGGGGCCGTTCCTCGGGGCATGACGCTGGCTCCTGCACAG | 176 |
| | ATCCTGCTCCTCTGTGGCCTTCCTGGGCTGCCCTCCCCTCCTCCGGGACT | |
| | GCTCTGGACTGACACTGCTCAGGTTCGGATTCCCTCAAAGACTTTGGGA | |
| | GACAAGACTTGGTCCCCCTTTTACAAACAAGGGAACGGAGGCTCTAGA | |
| | ACTGACTTCCTGAAAGGCTTGGATCCAAAGCTCCCTCAGTTCAGCGGCC | |
| | ACGTCTATTTCCCTCAGACACAGGGATCCTTGAACCTGTGGGCTGTATC | |
| | TCCCCGCGGACTTGGAAGAATCCCAAGAGAGTGGGGCTCCCACAGGCT | |
| | GGAGTGCAATGGTGTGATCTCGGCTCACTGCAACCTCCACCTCCCAGGT | |
| | TCAAGCTATTCTCCTGCCTCAGCCTCCTGAGTAGCTGGGATTACAGATC | |
| | CTGGTGGCTGTGGTCGGTAATTCCAGCTTCGTGCTGGCTACAGGTGGAT | |
| | GATGCCCACCTGGCTGCCGATGACCTCTGCACCAAGTGAGGCTGGGTCT | |
| | CTGGAGCTGCCCCAGGGGCTGGACAAGCTGACCCTGGCCGGGGCCAAC | |
| | CTGGAGATGCAGATTGAGAACCTCAAGGAGGACCTGGTCTACCTGAAG | |
| | AAGAACCACAAGCAGGAAATGAACGTCCTTTGAGGTCAGGTGGATGAG | |
| | GATGTCAGTGTGAAGATGGACACTGTGCCTGGAGTGAACCTGAGCTGC | |
| | ATCCTGAATGAGATGCGTGACCAGGACAAGACATTGGTGGAGAAGAGC | |
| | TGCAAGGATGCCGAGGGCTGGTTCTTCAGCATGGTGGGTGGCCGTGCGT | |
| | AAGCAGGTGTGTACACGTGTGGGCACATGTGCTGCATGCTGGTGCAGCT | |
| | GGAGCACTGGCAGATCCACAGGCTGTCCCAGTTGGAAGGACTTTTGGA | |
| | AACCAGTTGGACCAGCCCCTCATGTTTTAGATGTAAAACGTGAGGCTCA | |
| | GAGAGGACTCAAGCTCACACAGCCCTTCACTGTGGCCTGCAAAATAGA | |
| | TCCAGGTCTCTACAAGTCTGGTCTTGGGTTTCCACCACAGCTGTTTACAG | |
| | GATGTGCGTATTTGAATACATATGTATACCCTTGGCAAGCACAGGCTGA | |
| | GTATCTCCGGTATCCTAGGGACAGCAACAGGCGCAAAAGAATAACACC | |
| | CAGTGCCTGTCTTTGAGGTGCTGCAGTTCAGTAGGAAAAAGAAATGCA | |
| | AATGACCGCAGAGCAGGCTGAATTCCTCCAAGTTCCAATGTGGGTGCA | |
| | GAGGCTCTCTGTGTGCAGAAAGAGGGGCTGAACTGCGAGGTGGCCACC | |
| | AACACAGAGGCCCTGCAGAGTGGCTGGATAGAGATATGGAGCTCTACG | |
| | TCTCTGTGCAGAACCTGAGCCGTCCCAGCTCAGCAAGAAAGCATCGCTG | |
| | GAGGGCAGCCTGGTGGAGATGGAGGTGTGTTACAGGACCCTGCCGGCC | |
| | CAGCTGCAGGGGCTTAACAGAAGCATGGAGCAGCAGCTGTGCGAGCTC | |
| | TGCTGCGACACGGAGCACCAGGACCACAAGCACAGGTCCTTCTGGACG | |
| | TGAAGACGTGGCTGGAGCAGGAGATCGCCACCTACCGCCGCTTGCTGG | |
| | AGGTTGAGGACGCCCAGAGGTGATACTGACGATGCAGGCTGGAGTCTG | |
| | GCTGAGGAGCCTTGAATGCCAAGTTAAAGCGTCTGGACTAGATCACGT | |
| | AGGCAATGGGGAGCCATGGAGGGATTTGGAGCAGGAGAGTGAAATGA | |
| | ACATCAAGAGATTTTAGAACATTCACTCTGGCTGCAGAGGGAGAAATG | |
| | GATCAGAGGGGTCAGGGCGGGGCCAGAGAGATGTGTCAGGGGGCTGG | |
| | AGCAGGGAGTCTGGCCAGAGAAGTCCCGTGCGGTGGTGGGTAGTGGGG | |
| | CAGGGGAAGGAAGGTGGTGCACGCAGAAGAGAGGTTATAGCTCAAAA | |
| | CAGCGGGACTGGATGCCTGGATCTCGGGGTAAGCATGGCTCACAGTCA | |
| | GGACTCAGTAAGTGTCGGGAGAACACATGAAGGAGCAGGCATTGATGG | |
| | CCCTGGGTTTCTGGTTCTGATGACTGTGTGAGTGGTGAAGAGCAAGGTG | |
| | GGTGGTGGTTGGGTTTGCAGTTGGGAAGGGTGATCAGGCCTTCAGCTGA | |
| | GAGTGTCCCGGAGTCTCCATGCTTAGTCACACGTTGCAGCTTTTTGCTCC | |
| | CCGGAAATGGTGAAGTCCATCTATAGTCTAACAACAGTCTCTCCTGCTT | |
| | TAATTGGGTCTATTTGTTGGGCCCTCTGGGTTATGGAAAAACCACTTGC | |
| | TCAGCTTCTCCTTGTAAATTCCTGGTGAGTAGCCACAGAGTGCCGCCAG | |
| | ACCTACTGCTGTGCTGTTTCTTTTTCTTCTTCCTGCTGTGCTGAACCCCTG | |
| | CCCTTTCATTCTTGGGCCTGCGCTAATTTCTGTGCATTCCCAACTGTGAT | |
| | TTTTCACCAATTTAGGGGAACCTCCTCTGCCAGGGCCTACTTCTCCCCAG | |
| | CAGTGCTTGCAGGTGCCTGGGCTGGCTGGCATCCCTGGGCTGATGGGTG | |
| | CTTCTCTCCCTGCAGGCTGGCCACTCAGTACTCCTTGTCCCTGGCCTCGC | |
| | AGCCCACCCGGGAAGCCACAGTGACCAGCCACCAGGTGTGCCATCGTG | |
| | GAGGAAGTCCAGGTTGGAGAGGTGGTCTTCTTCTGTGAGCAGGTCCACT | |
| | TCTCCACCCACTGAGACCCCTTTCTGTCTGCGACAGCCCCACCTCGAGG | |
| | GCCACGGCACAGCCATCAGCTCCAGCTCCCAGCATGCTACTGCCACGCC | |
| | CCGAGTGTCCGTCTGGGCCCCGGTGCATGGCCTGTTGTCTTTCTGTATCT | |
| | ACTTTCTGCAGCCCCTCACTGAGGAGGCCTCCTGGGTTTGTCCAGTGCC | |
| | TACTATTAAAGCTTTGCTCCAAGTTC | |
| M21389 | GCATCCTTTTTGGGCTGCTCACAGCCCCCAGCCTCTATGGTGAAGACAT | 177 |
| | ACTTGCTAGCAGCGTCACCAACTTGCTGCCAAGAGATCAGTGCTGCAAG | |
| | GCAAGGTTATTTCTAACTGAGCAGAGCCTGCCAGGAAGAAAGCGTTTG | |
| | CACCCCACACCACTGTGCAGGTGTGACCGGTGAGCTCACAGCTGCCCCC | |
| | CAGGCATGCCCAGCCCACTTAATCATTCACAGCTCGACAGCTCTCTCGC | |
| | CCAGCCCAGTTCTGGAAGGGATAAAAAGGGGGCATCACCGTTCCTGGG | |
| | TAACAGAGCCACCTTCTGCGTCCTGCTGAGCTCTGTTCTCTCCAGCACCT | |
| | CCCAACCCACTAGTGCCTGGTTCTCTTGCTCCACCAGGAACAAGCCACC | |
| | ATGTCTCGCCAGTCAAGTGTGTCCTTCCGGAGCGGGGGCAGTCGTAGCT | |
| | TCAGCACCGCCTCTGCCATCACCCCGTCTGTCTCCCGCACCAGCTTCACC | |
| | TCCGTGTCCCGGTCCGGGGGTGGCGGTGGTGGTGGCTTCGGCAGGGTCA | |
| | GCCTTGCGGGTGCTTGTGGAGTGGGTGGCTATGGCAGCCGGAGCCTCTA | |
| | CAACCTGGGGGGCTCCAAGAGGATATCCATCAGCACTAGAGGAGGCAG | |
| | CTTCAGGAACCGGTTTGGTGCTGGTGCTGGAGGCGGCTATGGCTTTGGA | |
| | GGTGGTGCCGGTAGTGGATTTGGTTTCGGCGGTGGAGCTGGTGGTGGCT | |
| | TTGGGCTCGGTGGCGGAGCTGGCTTTGGAGGTGGCTTCGGTGGCCCTGG | |
| | CTTTCCTGTCTGCCCTCCTGGAGGTATCCAAGAGGTCACTGTCAACCAG | |
| | AGTCTCCTGACTCCCCTCAACCTGCAAATCGACCCCAGCATCCAGAGGG | |
| | TGAGGACCGAGGAGCGCGAGCAGATCAAGACCCTCAACAATAAGTTTG | |
| | CCTCCTTCATCGACAAGGTGCGGTTCCTGGAGCAGCAGAACAAGGTTCT | |
| | GGACACCAAGTGGACCCTGCTGCAGGAGCAGGGCACCAAGACTGTGAG | |
| | GCAGAACCTGGAGCCGTTGTTCGAGCAGTACATCAACAACCTCAGGAG | |
| | GCAGCTGGACAGCATCGTGGGGGAACGGGGCCGCCTGGACTCAGAGCT | |
| | GAGAAACATGCAGGACCTGGTGGAAGACTTCAAGAACAAGTATGAGGA | |
| | TGAAATCAACAAGCGTACCACTGCTGAGAATGAGTTTGTGATGCTGAA | |
| | GAAGGATGTAGATGCTGCCTACATGAACAAGGTGGAGCTGGAGGCCAA | |
| | GGTTGATGCACTGATGGATGAGATTAACTTCATGAAGATGTTCTTTGAT | |
| | GCGGAGCTGTCCCAGATGCAGACGCATGTCTCTGACACCTCAGTGGTCC | |
| | TCTCCATGGACAACAACCGCAACCTGGACCTGGATAGCATCATCGCTGA | |
| | GGTCAAGGCCCAGTATGAGGAGATTGCCAACCGCAGCCGGACAGAAGC | |
| | CGAGTCCTGGTATCAGACCAAGTATGAGGAGCTGCAGCAGACAGCTGG | |
| | CCGGCATGGCGATGACCTCCGCAACACCAAGCATGAGATCACAGAGAT | |
| | GAACCGGATGATCCAGAGGCTGAGAGCCGAGATTGACAATGTCAAGAA | |
| | ACAGTGCGCCAATCTGCAGAACGCCATTGCGGATGCCGAGCAGCGTGG | |
| | GGAGCTGGCCCTCAAGGATGCCAGGAACAAGCTGGCCGAGCTGGAGGA | |
| | GGCCCTGCAGAAGGCCAAGCAGGACATGGCCCGGCTGCTGCGTGAGTA | |
| | CCAGGAGCTCATGAACACCAAGCTGGCCCTGGACGTGGAGATCGCCAC | |
| | TTACCGCAAGCTGCTGGAGGGCGAGGAATGCAGACTCAGTGGAGAAGG | |
| | AGTTGGACCAGTCAACATCTCTGTTGTCACAAGCAGTGTTTCCTCTGGA | |
| | TATGGCAGTGGCAGTGGCTATGGCGGTGGCCTCGGTGGAGGTCTTGGCG | |
| | GCGGCCTCGGTGGAGGTCTTGCCGGAGGTAGCAGTGGAAGCTACTACT | |
| | CCAGCAGCAGTGGGGGTGTCGGCCTAGGTGGTGGGCTCAGTGTGGGGG | |
| | GCTCTGGCTTCAGTGCAAGCAGTGGCCGAGGGCTGGGGGTGGGCTTTG | |
| | GCAGTGGCGGGGGTAGCAGCTCCAGCGTCAAATTTGTCTCCACCACCTC | |
| | CTCCTCCCGGAAGAGCTTCAAGAGCTAAGAACCTGCTGCAAGTCACTGC | |
| | CTTCCAAGTGCAGCAACCCAGCCCATGGAGATTGCCTCTTCTAGGCAGT | |
| | TGCTCAAGCCATGTTTTATCCTTTTCTGGAGAGTAGTCTAGACCAAGCC | |
| | AATTGCAGAACCACATTCTTTGGTTCCCAGGAGAGCCCCATTCCCAGCC | |
| | CCTGGTCTCCCGTGCCGCAGTTCTATATTCTGCTTCAAATCAGCCTTCAG | |
| | GTTTCCCACAGCATGGCCCCTGCTGACACGAGAACCCAAAGTTTTCCCA | |
| | AATCTAAATCATCAAAACAGAATCCCCACCCCAATCCCAAATTTTGTTT | |
| | TGGTTCTAACTACCTCCAGAATGTGTTCAATAAAATGCTTTTATAATAT | |
| NM_001123066 | GGACGGCCGAGCGGCAGGGCGCTCGCGCGCGCCCACTAGTGGCCGGAG | 178 |
| | GAGAAGGCTCCCGCGGAGGCCGCGCTGCCCGCCCCCTCCCCTGGGGAG | |
| | GCTCGCGTTCCCGCTGCTCGCGCCTGCGCCGCCCGCCGGCCTCAGGAAC | |
| | GCGCCCTCTTCGCCGGCGCGCGCCCTCGCAGTCACCGCCACCCACCAGC | |
| | TCCGGCACCAACAGCAGCGCCGCTGCCACCGCCCACCTTCTGCCGCCGC | |
| | CACCACAGCCACCTTCTCCTCCTCCGCTGTCCTCTCCCGTCCTCGCCTCT | |
| | GTCGACTATCAGGTGAACTTTGAACCAGGATGGCTGAGCCCCGCCAGG | |
| | AGTTCGAAGTGATGGAAGATCACGCTGGGACGTACGGGTTGGGGGACA | |
| | GGAAAGATCAGGGGGGCTACACCATGCACCAAGACCAAGAGGGTGAC | |
| | ACGGACGCTGGCCTGAAAGAATCTCCCCTGCAGACCCCCACTGAGGAC | |
| | GGATCTGAGGAACCGGGCTCTGAAACCTCTGATGCTAAGAGCACTCCA | |
| | ACAGCGGAAGATGTGACAGCACCCTTAGTGGATGAGGGAGCTCCCGGC | |
| | AAGCAGGCTGCCGCGCAGCCCCACACGGAGATCCCAGAAGGAACCACA | |
| | GCTGAAGAAGCAGGCATTGGAGACACCCCCAGCCTGGAAGACGAAGCT | |
| | GCTGGTCACGTGACCCAAGAGCCTGAAAGTGGTAAGGTGGTCCAGGAA | |
| | GGCTTCCTCCGAGAGCCAGGCCCCCCAGGTCTGAGCCACCAGCTCATGT | |
| | CCGGCATGCCTGGGGCTCCCCTCCTGCCTGAGGGCCCCAGAGAGGCCAC | |
| | ACGCCAACCTTCGGGGACAGGACCTGAGGACACAGAGGGCGGCCGCCA | |
| | CGCCCCTGAGCTGCTCAAGCACCAGCTTCTAGGAGACCTGCACCAGGA | |
| | GGGGCCGCCGCTGAAGGGGGCAGGGGGCAAAGAGAGGCCGGGGAGCA | |
| | AGGAGGAGGTGGATGAAGACCGCGACGTCGATGAGTCCTCCCCCCAAG | |
| | ACTCCCCTCCCTCCAAGGCCTCCCCAGCCCAAGATGGGCGGCCTCCCCA | |
| | GACAGCCGCCAGAGAAGCCACCAGCATCCCAGGCTTCCCAGCGGAGGG | |
| | TGCCATCCCCCTCCCTGTGGATTTCCTCTCCAAAGTTTCCACAGAGATCC | |
| | CAGCCTCAGAGCCCGACGGGCCCAGTGTAGGGCGGGCCAAAGGGCAGG | |
| | ATGCCCCCCTGGAGTTCACGTTTCACGTGGAAATCACACCCAACGTGCA | |
| | GAAGGAGCAGGCGCACTCGGAGGAGCATTTGGGAAGGGCTGCATTTCC | |
| | AGGGGCCCCTGGAGAGGGGCCAGAGGCCCGGGGCCCCTCTTTGGGAGA | |
| | GGACACAAAAGAGGCTGACCTTCCAGAGCCCTCTGAAAAGCAGCCTGC | |
| | TGCTGCTCCGCGGGGGAAGCCCGTCAGCCGGGTCCCTCAACTCAAAGCT | |
| | CGCATGGTCAGTAAAAGCAAAGACGGGACTGGAAGCGATGACAAAAA | |
| | AGCCAAGACATCCACACGTTCCTCTGCTAAAACCTTGAAAAATAGGCCT | |
| | TGCCTTAGCCCCAAACACCCCACTCCTGGTAGCTCAGACCCTCTGATCC | |
| | AACCCTCCAGCCCTGCTGTGTGCCCAGAGCCACCTTCCTCTCCTAAATA | |
| | CGTCTCTTCTGTCACTTCCCGAACTGGCAGTTCTGGAGCAAAGGAGATG | |
| | AAACTCAAGGGGGCTGATGGTAAAACGAAGATCGCCACACCGCGGGGA | |
| | GCAGCCCCTCCAGGCCAGAAGGGCCAGGCCAACGCCACCAGGATTCCA | |
| | GCAAAAACCCCGCCCGCTCCAAAGACACCACCCAGCTCTGCGACTAAG | |
| | CAAGTCCAGAGAAGACCACCCCCTGCAGGGCCCAGATCTGAGAGAGGT | |
| | GAACCTCCAAAATCAGGGGATCGCAGCGGCTACAGCAGCCCCGGCTCC | |
| | CCAGGCACTCCCGGCAGCCGCTCCCGCACCCCGTCCCTTCCAACCCCAC | |
| | CCACCCGGGAGCCCAAGAAGGTGGCAGTGGTCCGTACTCCACCCAAGT | |
| | CGCCGTCTTCCGCCAAGAGCCGCCTGCAGACAGCCCCCGTGCCCATGCC | |
| | AGACCTGAAGAATGTCAAGTCCAAGATCGGCTCCACTGAGAACCTGAA | |
| | GCACCAGCCGGGAGGCGGGAAGGTGCAGATAATTAATAAGAAGCTGGA | |
| | TCTTAGCAACGTCCAGTCCAAGTGTGGCTCAAAGGATAATATCAAACAC | |
| | GTCCCGGGAGGCGGCAGTGTGCAAATAGTCTACAAACCAGTTGACCTG | |
| | AGCAAGGTGACCTCCAAGTGTGGCTCATTAGGCAACATCCATCATAAAC | |
| | CAGGAGGTGGCCAGGTGGAAGTAAAATCTGAGAAGCTTGACTTCAAGG | |
| | ACAGAGTCCAGTCGAAGATTGGGTCCCTGGACAATATCACCCACGTCCC | |
| | TGGCGGAGGAAATAAAAAGATTGAAACCCACAAGCTGACCTTCCGCGA | |
| | GAACGCCAAAGCCAAGACAGACCACGGGGCGGAGATCGTGTACAAGTC | |
| | GCCAGTGGTGTCTGGGGACACGTCTCCACGGCATCTCAGCAATGTCTCC | |
| | TCCACCGGCAGCATCGACATGGTAGACTCGCCCCAGCTCGCCACGCTAG | |
| | CTGACGAGGTGTCTGCCTCCCTGGCCAAGCAGGGTTTGTGATCAGGCCC | |
| | CTGGGGCGGTCAATAATTGTGGAGAGGAGAGAATGAGAGAGTGTGGAA | |
| | AAAAAAAGAATAATGACCCGGCCCCCGCCCTCTGCCCCCAGCTGCTCCT | |
| | CGCAGTTCGGTTAATTGGTTAATCACTTAACCTGCTTTTGTCACTCGGCT | |
| | TTGGCTCGGGACTTCAAAATCAGTGATGGGAGTAAGAGCAAATTTCATC | |
| | TTTCCAAATTGATGGGTGGGCTAGTAATAAAATATTTAAAAAAAAACAT | |
| | TCAAAAACATGGCCACATCCAACATTTCCTCAGGCAATTCCTTTTGATT | |
| | CTTTTTTCTTCCCCCTCCATGTAGAAGAGGGAGAAGGAGAGGCTCTGAA | |
| | AGCTGCTTCTGGGGGATTTCAAGGGACTGGGGGTGCCAACCACCTCTGG | |
| | CCCTGTTGTGGGGGTGTCACAGAGGCAGTGGCAGCAACAAAGGATTTG | |
| | AAACTTGGTGTGTTCGTGGAGCCACAGGCAGACGATGTCAACCTTGTGT | |
| | GAGTGTGACGGGGGTTGGGGTGGGGCGGGAGGCCACGGGGGAGGCCG | |
| | AGGCAGGGGCTGGGCAGAGGGGAGAGGAAGCACAAGAAGTGGGAGTG | |
| | GGAGAGGAAGCCACGTGCTGGAGAGTAGACATCCCCCTCCTTGCCGCT | |
| | GGGAGAGCCAAGGCCTATGCCACCTGCAGCGTCTGAGCGGCCGCCTGT | |
| | CCTTGGTGGCCGGGGGTGGGGGCCTGCTGTGGGTCAGTGTGCCACCCTC | |
| | TGCAGGGCAGCCTGTGGGAGAAGGGACAGCGGGTAAAAAGAGAAGGC | |
| | AAGCTGGCAGGAGGGTGGCACTTCGTGGATGACCTCCTTAGAAAAGAC | |
| | TGACCTTGATGTCTTGAGAGCGCTGGCCTCTTCCTCCCTCCCTGCAGGGT | |
| | AGGGGGCCTGAGTTGAGGGGCTTCCCTCTGCTCCACAGAAACCCTGTTT | |
| | TATTGAGTTCTGAAGGTTGGAACTGCTGCCATGATTTTGGCCACTTTGC | |
| | AGACCTGGGACTTTAGGGCTAACCAGTTCTCTTTGTAAGGACTTGTGCC | |
| | TCTTGGGAGACGTCCACCCGTTTCCAAGCCTGGGCCACTGGCATCTCTG | |
| | GAGTGTGTGGGGGTCTGGGAGGCAGGTCCCGAGCCCCCTGTCCTTCCCA | |
| | CGGCCACTGCAGTCACCCCGTCTGCGCCGCTGTGCTGTTGTCTGCCGTG | |
| | AGAGCCCAATCACTGCCTATACCCCTCATCACACGTCACAATGTCCCGA | |
| | ATTCCCAGCCTCACCACCCCTTCTCAGTAATGACCCTGGTTGGTTGCAG | |
| | GAGGTACCTACTCCATACTGAGGGTGAAATTAAGGGAAGGCAAAGTCC | |
| | AGGCACAAGAGTGGGACCCCAGCCTCTCACTCTCAGTTCCACTCATCCA | |
| | ACTGGGACCCTCACCACGAATCTCATGATCTGATTCGGTTCCCTGTCTCC | |
| | TCCTCCCGTCACAGATGTGAGCCAGGGCACTGCTCAGCTGTGACCCTAG | |
| | GTGTTTCTGCCTTGTTGACATGGAGAGAGCCCTTTCCCCTGAGAAGGCC | |
| | TGGCCCCTTCCTGTGCTGAGCCCACAGCAGCAGGCTGGGTGTCTTGGTT | |
| | GTCAGTGGTGGCACCAGGATGGAAGGGCAAGGCACCCAGGGCAGGCCC | |
| | ACAGTCCCGCTGTCCCCCACTTGCACCCTAGCTTGTAGCTGCCAACCTC | |
| | CCAGACAGCCCAGCCCGCTGCTCAGCTCCACATGCATAGTATCAGCCCT | |
| | CCACACCCGACAAAGGGGAACACACCCCCTTGGAAATGGTTCTTTTCCC | |
| | CCAGTCCCAGCTGGAAGCCATGCTGTCTGTTCTGCTGGAGCAGCTGAAC | |
| | ATATACATAGATGTTGCCCTGCCCTCCCCATCTGCACCCTGTTGAGTTGT | |
| | AGTTGGATTTGTCTGTTTATGCTTGGATTCACCAGAGTGACTATGATAGT | |
| | GAAAAGAAAAAAAAAAAAAAAAAAGGACGCATGTATCTTGAAATGCTT | |
| | GTAAAGAGGTTTCTAACCCACCCTCACGAGGTGTCTCTCACCCCCACAC | |
| | TGGGACTCGTGTGGCCTGTGTGGTGCCACCCTGCTGGGGCCTCCCAAGT | |
| | TTTGAAAGGCTTTCCTCAGCACCTGGGACCCAACAGAGACCAGCTTCTA | |
| | GCAGCTAAGGAGGCCGTTCAGCTGTGACGAAGGCCTGAAGCACAGGAT | |
| | TAGGACTGAAGCGATGATGTCCCCTTCCCTACTTCCCCTTGGGGCTCCCT | |
| | GTGTCAGGGCACAGACTAGGTCTTGTGGCTGGTCTGGCTTGCGGCGCGA | |
| | GGATGGTTCTCTCTGGTCATAGCCCGAAGTCTCATGGCAGTCCCAAAGG | |
| | AGGCTTACAACTCCTGCATCACAAGAAAAAGGAAGCCACTGCCAGCTG | |
| | GGGGGATCTGCAGCTCCCAGAAGCTCCGTGAGCCTCAGCCACCCCTCAG | |
| | ACTGGGTTCCTCTCCAAGCTCGCCCTCTGGAGGGGCAGCGCAGCCTCCC | |
| | ACCAAGGGCCCTGCGACCACAGCAGGGATTGGGATGAATTGCCTGTCC | |
| | TGGATCTGCTCTAGAGGCCCAAGCTGCCTGCCTGAGGAAGGATGACTTG | |
| | ACAAGTCAGGAGACACTGTTCCCAAAGCCTTGACCAGAGCACCTCAGC | |
| | CCGCTGACCTTGCACAAACTCCATCTGCTGCCATGAGAAAAGGGAAGC | |
| | CGCCTTTGCAAAACATTGCTGCCTAAAGAAACTCAGCAGCCTCAGGCCC | |
| | AATTCTGCCACTTCTGGTTTGGGTACAGTTAAAGGCAACCCTGAGGGAC | |
| | TTGGCAGTAGAAATCCAGGGCCTCCCCTGGGGCTGGCAGCTTCGTGTGC | |
| | AGCTAGAGCTTTACCTGAAAGGAAGTCTCTGGGCCCAGAACTCTCCACC | |
| | AAGAGCCTCCCTGCCGTTCGCTGAGTCCCAGCAATTCTCCTAAGTTGAA | |
| | GGGATCTGAGAAGGAGAAGGAAATGTGGGGTAGATTTGGTGGTGGTTA | |
| | GAGATATGCCCCCCTCATTACTGCCAACAGTTTCGGCTGCATTTCTTCAC | |
| | GCACCTCGGTTCCTCTTCCTGAAGTTCTTGTGCCCTGCTCTTCAGCACCA | |
| | TGGGCCTTCTTATACGGAAGGCTCTGGGATCTCCCCCTTGTGGGGCAGG | |
| | CTCTTGGGGCCAGCCTAAGATCATGGTTTAGGGTGATCAGTGCTGGCAG | |
| | ATAAATTGAAAAGGCACGCTGGCTTGTGATCTTAAATGAGGACAATCCC | |
| | CCCAGGGCTGGGCACTCCTCCCCTCCCCTCACTTCTCCCACCTGCAGAG | |
| | CCAGTGTCCTTGGGTGGGCTAGATAGGATATACTGTATGCCGGCTCCTT | |
| | CAAGCTGCTGACTCACTTTATCAATAGTTCCATTTAAATTGACTTCAGTG | |
| | GTGAGACTGTATCCTGTTTGCTATTGCTTGTTGTGCTATGGGGGGAGGG | |
| | GGGAGGAATGTGTAAGATAGTTAACATGGGCAAAGGGAGATCTTGGGG | |
| | TGCAGCACTTAAACTGCCTCGTAACCCTTTTCATGATTTCAACCACATTT | |
| | GCTAGAGGGAGGGAGCAGCCACGGAGTTAGAGGCCCTTGGGGTTTCTC | |
| | TTTTCCACTGACAGGCTTTCCCAGGCAGCTGGCTAGTTCATTCCCTCCCC | |
| | AGCCAGGTGCAGGCGTAGGAATATGGACATCTGGTTGCTTTGGCCTGCT | |
| | GCCCTCTTTCAGGGGTCCTAAGCCCACAATCATGCCTCCCTAAGACCTT | |
| | GGCATCCTTCCCTCTAAGCCGTTGGCACCTCTGTGCCACCTCTCACACTG | |
| | GCTCCAGACACACAGCCTGTGCTTTTGGAGCTGAGATCACTCGCTTCAC | |
| | CCTCCTCATCTTTGTTCTCCAAGTAAAGCCACGAGGTCGGGGCGAGGGC | |
| | AGAGGTGATCACCTGCGTGTCCCATCTACAGACCTGCAGCTTCATAAAA | |
| | CTTCTGATTTCTCTTCAGCTTTGAAAAGGGTTACCCTGGGCACTGGCCTA | |
| | GAGCCTCACCTCCTAATAGACTTAGCCCCATGAGTTTGCCATGTTGAGC | |
| | AGGACTATTTCTGGCACTTGCAAGTCCCATGATTTCTTCGGTAATTCTGA | |
| | GGGTGGGGGGAGGGACATGAAATCATCTTAGCTTAGCTTTCTGTCTGTG | |
| | AATGTCTATATAGTGTATTGTGTGTTTTAACAAATGATTTACACTGACTG | |
| | TTGCTGTAAAAGTGAATTTGGAAATAAAGTTATTACTCTGATTAAA | |
| M92424 | GCACCGCGCGAGCTTGGCTGCTTCTGGGGCCTGTGTGGCCCTGTGTGTC | 179 |
| | GGAAAGATGGAGCAAGAAGCCGAGCCCGAGGGGCGGCCGCGACCCCT | |
| | CTGACCGAGATCCTGCTGCTTTCGCAGCCAGGAGCACCGTCCCTCCCCG | |
| | GATTAGTGCGTACGAGCGCCCAGTGCCCTGGCCCGGAGAGTGGAATGA | |
| | TCCCCGAGGCCCAGGGCGTCGTGCTTCCGCAGTAGTCAGTCCCCGTGAA | |
| | GGAAACTGGGGAGTCTTGAGGGACCCCCGACTCCAAGCGCGAAAACCC | |
| | CGGATGGTGAGGAGCAGGCAAATGTGCAATACCAACATGTCTGTACCT | |
| | ACTGATGGTGCTGTAACCACCTCACAGATTCCAGCTTCGGAACAAGAGA | |
| | CCCTGGTTAGACCAAAGCCATTGCTTTTGAAGTTATTAAAGTCTGTTGG | |
| | TGCACAAAAAGACACTTATACTATGAAAGAGGTTCTTTTTTATCTTGGC | |
| | CAGTATATTATGACTAAACGATTATATGATGAGAAGCAACAACATATTG | |
| | TATATTGTTCAAATGATCTTCTAGGAGATTTGTTTGGCGTGCCAAGCTTC | |
| | TCTGTGAAAGAGCACAGGAAAATATATACCATGATCTACAGGAACTTG | |
| | GTAGTAGTCAATCAGCAGGAATCATCGGACTCAGGTACATCTGTGAGTG | |
| | AGAACAGGTGTCACCTTGAAGGTGGGAGTGATCAAAAGGACCTTGTAC | |
| | AAGAGCTTCAGGAAGAGAAACCTTCATCTTCACATTTGGTTTCTAGACC | |
| | ATCTACCTCATCTAGAAGGAGAGCAATTAGTGAGACAGAAGAAAATTC | |
| | AGATGAATTATCTGGTGAACGACAAAGAAAACGCCACAAATCTGATAG | |
| | TATTTCCCTTTCCTTTGATGAAAGCCTGGCTCTGTGTGTAATAAGGGAG | |
| | ATATGTTGTGAAAGAAGCAGTAGCAGTGAATCTACAGGGACGCCATCG | |
| | AATCCGGATCTTGATGCTGGTGTAAGTGAACATTCAGGTGATTGGTTGG | |
| | ATCAGGATTCAGTTTCAGATCAGTTTAGTGTAGAATTTGAAGTTGAATC | |
| | TCTCGACTCAGAAGATTATAGCCTTAGTGAAGAAGGACAAGAACTCTC | |
| | AGATGAAGATGATGAGGTATATCAAGTTACTGTGTATCAGGCAGGGGA | |
| | GAGTGATACAGATTCATTTGAAGAAGATCCTGAAATTTCCTTAGCTGAC | |
| | TATTGGAAATGCACTTCATGCAATGAAATGAATCCCCCCCTTCCATCAC | |
| | ATTGCAACAGATGTTGGGCCCTTCGTGAGAATTGGCTTCCTGAAGATAA | |
| | AGGGAAAGATAAAGGGGAAATCTCTGAGAAAGCCAAACTGGAAAACT | |
| | CAACACAAGCTGAAGAGGGCTTTGATGTTCCTGATTGTAAAAAAACTAT | |
| | AGTGAATGATTCCAGAGAGTCATGTGTTGAGGAAAATGATGATAAAAT | |
| | TACACAAGCTTCACAATCACAAGAAAGTGAAGACTATTCTCAGCCATCA | |
| | ACTTCTAGTAGCATTATTTATAGCAGCCAAGAAGATGTGAAAGAGTTTG | |
| | AAAGGGAAGAAACCCAAGACAAAGAAGAGAGTGTGGAATCTAGTTTGC | |
| | CCCTTAATGCCATTGAACCTTGTGTGATTTGTCAAGGTCGACCTAAAAA | |
| | TGGTTGCATTGTCCATGGCAAAACAGGACATCTTATGGCCTGCTTTACA | |
| | TGTGCAAAGAAGCTAAAGAAAAGGAATAAGCCCTGCCCAGTATGTAGA | |
| | CAACCAATTCAAATGATTGTGCTAACTTATTTCCCCTAGTTGACCTGTCT | |
| | ATAAGAGAATTATATATTTCTAACTATATAACCCTAGGAATTTAGACAA | |
| | CCTGAAATTTATTCACATATATCAAAGTGAGAAAATGCCTCAATTCACA | |
| | TAGATTTCTTCTCTTTAGTATAATTGACCTACTTTGGTAGTGGAATAGTG | |
| | AATACTTACTATAATTTGACTTGAATATGTAGCTCATCCTTTACACCAAC | |
| | TCCTAATTTTAAATAATTTCTACTCTGTCTTAAATGAGAAGTACTTGGTT | |
| | TTTTTTTTCTTAAATATGTATATGACATTTAAATGTAACTTATTATTTTTT | |
| | TTGAGACCGAGTCTTGCTCTGTTACCCAGGCTGGAGTGCAGTGGGTGAT | |
| | CTTGGCTCACTGCAAGCTCTGCCCTCCCCGGGTTCGCACCATTCTCCTGC | |
| | CTCAGCCTCCCAATTAGCTTGGCCTACAGTCATCTGCCACCACACCTGG | |
| | CTAATTTTTTGTACTTTTAGTAGAGACAGGGTTTCACCGTGTTAGCCAGG | |
| | ATGGTCTCGATCTCCTGACCTCGTGATCCGCCCACCTCGGCCTCCCAAA | |
| | GTGCTGGGATTACAGGCATGAGCCACCG | |
| NM_014791 | GAGATTTGATTCCCTTGGCGGGCGGAAGCGGCCACAACCCGGCGATCG | 180 |
| | AAAAGATTCTTAGGAACGCCGTACCAGCCGCGTCTCTCAGGACAGCAG | |
| | GCCCCTGTCCTTCTGTCGGGCGCCGCTCAGCCGTGCCCTCCGCCCCTCA | |
| | GGTTCTTTTTCTAATTCCAAATAAACTTGCAAGAGGACTATGAAAGATT | |
| | ATGATGAACTTCTCAAATATTATGAATTACATGAAACTATTGGGACAGG | |
| | TGGCTTTGCAAAGGTCAAACTTGCCTGCCATATCCTTACTGGAGAGATG | |
| | GTAGCTATAAAAATCATGGATAAAAACACACTAGGGAGTGATTTGCCC | |
| | CGGATCAAAACGGAGATTGAGGCCTTGAAGAACCTGAGACATCAGCAT | |
| | ATATGTCAACTCTACCATGTGCTAGAGACAGCCAACAAAATATTCATGG | |
| | TTCTTGAGTACTGCCCTGGAGGAGAGCTGTTTGACTATATAATTTCCCA | |
| | GGATCGCCTGTCAGAAGAGGAGACCCGGGTTGTCTTCCGTCAGATAGTA | |
| | TCTGCTGTTGCTTATGTGCACAGCCAGGGCTATGCTCACAGGGACCTCA | |
| | AGCCAGAAAATTTGCTGTTTGATGAATATCATAAATTAAAGCTGATTGA | |
| | CTTTGGTCTCTGTGCAAAACCCAAGGGTAACAAGGATTACCATCTACAG | |
| | ACATGCTGTGGGAGTCTGGCTTATGCAGCACCTGAGTTAATACAAGGCA | |
| | AATCATATCTTGGATCAGAGGCAGATGTTTGGAGCATGGGCATACTGTT | |
| | ATATGTTCTTATGTGTGGATTTCTACCATTTGATGATGATAATGTAATGG | |
| | CTTTATACAAGAAGATTATGAGAGGAAAATATGATGTTCCCAAGTGGCT | |
| | CTCTCCCAGTAGCATTCTGCTTCTTCAACAAATGCTGCAGGTGGACCCA | |
| | AAGAAACGGATTTCTATGAAAAATCTATTGAACCATCCCTGGATCATGC | |
| | AAGATTACAACTATCCTGTTGAGTGGCAAAGCAAGAATCCTTTTATTCA | |
| | CCTCGATGATGATTGCGTAACAGAACTTTCTGTACATCACAGAAACAAC | |
| | AGGCAAACAATGGAGGATTTAATTTCACTGTGGCAGTATGATCACCTCA | |
| | CGGCTACCTATCTTCTGCTTCTAGCCAAGAAGGCTCGGGGAAAACCAGT | |
| | TCGTTTAAGGCTTTCTTCTTTCTCCTGTGGACAAGCCAGTGCTACCCCAT | |
| | TCACAGACATCAAGTCAAATAATTGGAGTCTGGAAGATGTGACCGCAA | |
| | GTGATAAAAATTATGTGGCGGGATTAATAGACTATGATTGGTGTGAAG | |
| | ATGATTTATCAACAGGTGCTGCTACTCCCCGAACATCACAGTTTACCAA | |
| | GTACTGGACAGAATCAAATGGGGTGGAATCTAAATCATTAACTCCAGC | |
| | CTTATGCAGAACACCTGCAAATAAATTAAAGAACAAAGAAAATGTATA | |
| | TACTCCTAAGTCTGCTGTAAAGAATGAAGAGTACTTTATGTTTCCTGAG | |
| | CCAAAGACTCCAGTTAATAAGAACCAGCATAAGAGAGAAATACTCACT | |
| | ACGCCAAATCGTTACACTACACCCTCAAAAGCTAGAAACCAGTGCCTG | |
| | AAAGAAACTCCAATTAAAATACCAGTAAATTCAACAGGAACAGACAAG | |
| | TTAATGACAGGTGTCATTAGCCCTGAGAGGCGGTGCCGCTCAGTGGAAT | |
| | TGGATCTCAACCAAGCACATATGGAGGAGACTCCAAAAAGAAAGGGAG | |
| | CCAAAGTGTTTGGGAGCCTTGAAAGGGGGTTGGATAAGGTTATCACTGT | |
| | GCTCACCAGGAGCAAAAGGAAGGGTTCTGCCAGAGACGGGCCCAGAAG | |
| | ACTAAAGCTTCACTATAACGTGACTACAACTAGATTAGTGAATCCAGAT | |
| | CAACTGTTGAATGAAATAATGTCTATTCTTCCAAAGAAGCATGTTGACT | |
| | TTGTACAAAAGGGTTATACACTGAAGTGTCAAACACAGTCAGATTTTGG | |
| | GAAAGTGACAATGCAATTTGAATTAGAAGTGTGCCAGCTTCAAAAACC | |
| | CGATGTGGTGGGTATCAGGAGGCAGCGGCTTAAGGGCGATGCCTGGGT | |
| | TTACAAAAGATTAGTGGAAGACATCCTATCTAGCTGCAAGGTATAATTG | |
| | ATGGATTCTTCCATCCTGCCGGATGAGTGTGGGTGTGATACAGCCTACA | |
| | TAAAGACTGTTATGATCGCTTTGATTTTAAAGTTCATTGGAACTACCAA | |
| | CTTGTTTCTAAAGAGCTATCTTAAGACCAATATCTCTTTGTTTTTAAACA | |
| | AAAGATATTATTTTGTGTATGAATCTAAATCAAGCCCATCTGTCATTAT | |
| | GTTACTGTCTTTTTTAATCATGTGGTTTTGTATATTAATAATTGTTGACTT | |
| | TCTTAGATTCACTTCCATATGTGAATGTAAGCTCTTAACTATGTCTCTTT | |
| | GTAATGTGTAATTTCTTTCTGAAATAAAACCATTTGTGAATATAG | |
| BG765502 | GCAGCGGAGGAGCCCAGTCCACGATGGCCCGGTCCCTGGTGTGCCTTG | 181 |
| | GTGTCATCATCTTGCTGTCTGCCTTCTCCGGACCTGGTGTCAGGGGTGGT | |
| | CCTATGCCCAAGCTGGCTGACCGGAAGCTGTGTGCGGACCAGGAGTGC | |
| | AGCCACCCTATCTCCATGGCTGTGGCCCTTCAGGACTACATGGCCCCCG | |
| | ACTGCCGATTCCTGACCATTCACCGGGGCCAAGTGGTGTATGTCTTCTC | |
| | CAAGCTGAAGGGCCGTGGGCGGCTCTTCTGGGGAGGCAGCGTTCAGGG | |
| | AGATTACTATGGAGATCTGGCTGCTCGCCTGGGCTATTTCCCCAGTAGC | |
| | ATTGTCCGAGAGGACCAGACCCTGAAACCTGGCAAAGTCGATGTGAAG | |
| | ACAGACAAATGGGATTTCTACTGCCAGTGAGCTCAGCCTACCGCTGGCC | |
| | CTGCCGTTTCCCCTCCTTGGGTTTATGCAAATACAATCAGCCCAGTGCA | |
| | AAAAAAAAAAAAAAAAAAAAAAACTTCGGAGAAGAGATAGCAACAAA | |
| | AGGCCGCTTGTGTGAAGGCGCCAAAAGTTTTCGCCCAAGAGACCTTCGG | |
| | CCTCCCCCAGGGCGCGCGCAAAGGCGCCTTGTTTTGACAACCTCTTGGA | |
| | CAACCGGAGGGGCTACCGCCCGGAGACCCCTGTGGTGGACCCCCCGGG | |
| | CAACCCGGTGTGACAGGGTACTCACCCCCACGGCTTTGTCGGGGGTCCC | |
| | ACCAAAGGCCCCAAAGAGGCTCTTTCAAGGCACTATTCCTTGTTGTAGA | |
| | CCTTGTGTGTGCCACAGGCGCCAAAGAAACCTCGGGGGGCTAACAAAC | |
| | GCACGTGCTTGGCAGCTCCGAGAAGGCTCTCTCCCACCCGAGGGGTGG | |
| | ACGCAACAGGGGGAATGGGCCATCATATTGTTGCCCCCGGTGGGCACC | |
| | AACTCTTTTTCCCCCATAGAGAGGCCTTAGCACACTATGTGGGGCACGT | |
| | TATTGCCGCCTAGAGAAACCGAGCGCCAGAAAATTTCGAAGGGGGGGG | |
| | CGCTTCTCATCATTTTGCGCAAAACCCCCTTGTGGGAGTATGCCCCGAA | |
| | CTCCTCTGGAACACACAAGCGACACTTGCGCGGGGTCTGCAAAAAACC | |
| | TCCTGTTGGGAAGCCGGCTTCACN | |
| NM_002417 | TACCGGGCGGAGGTGAGCGCGGCGCCGGCTCCTCCTGCGGCGGACTTT | 182 |
| | GGGTGCGACTTGACGAGCGGTGGTTCGACAAGTGGCCTTGCGGGCCGG | |
| | ATCGTCCCAGTGGAAGAGTTGTAAATTTGCTTCTGGCCTTCCCCTACGG | |
| | ATTATACCTGGCCTTCCCCTACGGATTATACTCAACTTACTGTTTAGAAA | |
| | ATGTGGCCCACGAGACGCCTGGTTACTATCAAAAGGAGCGGGGTCGAC | |
| | GGTCCCCACTTTCCCCTGAGCCTCAGCACCTGCTTGTTTGGAAGGGGTA | |
| | TTGAATGTGACATCCGTATCCAGCTTCCTGTTGTGTCAAAACAACATTG | |
| | CAAAATTGAAATCCATGAGCAGGAGGCAATATTACATAATTTCAGTTCC | |
| | ACAAATCCAACACAAGTAAATGGGTCTGTTATTGATGAGCCTGTACGGC | |
| | TAAAACATGGAGATGTAATAACTATTATTGATCGTTCCTTCAGGTATGA | |
| | AAATGAAAGTCTTCAGAATGGAAGGAAGTCAACTGAATTTCCAAGAAA | |
| | AATACGTGAACAGGAGCCAGCACGTCGTGTCTCAAGATCTAGCTTCTCT | |
| | TCTGACCCTGATGAGAAAGCTCAAGATTCCAAGGCCTATTCAAAAATCA | |
| | CTGAAGGAAAAGTTTCAGGAAATCCTCAGGTACATATCAAGAATGTCA | |
| | AAGAAGACAGTACCGCAGATGACTCAAAAGACAGTGTTGCTCAGGGAA | |
| | CAACTAATGTTCATTCCTCAGAACATGCTGGACGTAATGGCAGAAATGC | |
| | AGCTGATCCCATTTCTGGGGATTTTAAAGAAATTTCCAGCGTTAAATTA | |
| | GTGAGCCGTTATGGAGAATTGAAGTCTGTTCCCACTACACAATGTCTTG | |
| | ACAATAGCAAAAAAAATGAATCTCCCTTTTGGAAGCTTTATGAGTCAGT | |
| | GAAGAAAGAGTTGGATGTAAAATCACAAAAAGAAAATGTCCTACAGTA | |
| | TTGTAGAAAATCTGGATTACAAACTGATTACGCAACAGAGAAAGAAAG | |
| | TGCTGATGGTTTACAGGGGGAGACCCAACTGTTGGTCTCGCGTAAGTCA | |
| | AGACCAAAATCTGGTGGGAGCGGCCACGCTGTGGCAGAGCCTGCTTCA | |
| | CCTGAACAAGAGCTTGACCAGAACAAGGGGAAGGGAAGAGACGTGGA | |
| | GTCTGTTCAGACTCCCAGCAAGGCTGTGGGCGCCAGCTTTCCTCTCTAT | |
| | GAGCCGGCTAAAATGAAGACCCCTGTACAATATTCACAGCAACAAAAT | |
| | TCTCCACAAAAACATAAGAACAAAGACCTGTATACTACTGGTAGAAGA | |
| | GAATCTGTGAATCTGGGTAAAAGTGAAGGCTTCAAGGCTGGTGATAAA | |
| | ACTCTTACTCCCAGGAAGCTTTCAACTAGAAATCGAACACCAGCTAAAG | |
| | TTGAAGATGCAGCTGACTCTGCCACTAAGCCAGAAAATCTCTCTTCCAA | |
| | AACCAGAGGAAGTATTCCTACAGATGTGGAAGTTCTGCCTACGGAAAC | |
| | TGAAATTCACAATGAGCCATTTTTAACTCTGTGGCTCACTCAAGTTGAG | |
| | AGGAAGATCCAAAAGGATTCCCTCAGCAAGCCTGAGAAATTGGGCACT | |
| | ACAGCTGGACAGATGTGCTCTGGGTTACCTGGTCTTAGTTCAGTTGATA | |
| | TCAACAACTTTGGTGATTCCATTAATGAGAGTGAGGGAATACCTTTGAA | |
| | AAGAAGGCGTGTGTCCTTTGGTGGGCACCTAAGACCTGAACTATTTGAT | |
| | GAAAACTTGCCTCCTAATACGCCTCTCAAAAGGGGAGAAGCCCCAACC | |
| | AAAAGAAAGTCTCTGGTAATGCACACTCCACCTGTCCTGAAGAAAATC | |
| | ATCAAGGAACAGCCTCAACCATCAGGAAAACAAGAGTCAGGTTCAGAA | |
| | ATCCATGTGGAAGTGAAGGCACAAAGCTTGGTTATAAGCCCTCCAGCTC | |
| | CTAGTCCTAGGAAAACTCCAGTTGCCAGTGATCAACGCCGTAGGTCCTG | |
| | CAAAACAGCCCCTGCTTCCAGCAGCAAATCTCAGACAGAGGTTCCTAA | |
| | GAGAGGAGGGAGAAAGAGTGGCAACCTGCCTTCAAAGAGAGTGTCTAT | |
| | CAGCCGAAGTCAACATGATATTTTACAGATGATATGTTCCAAAAGAAG | |
| | AAGTGGTGCTTCGGAAGCAAATCTGATTGTTGCAAAATCATGGGCAGAT | |
| | GTAGTAAAACTTGGTGCAAAACAAACACAAACTAAAGTCATAAAACAT | |
| | GGTCCTCAAAGGTCAATGAACAAAAGGCAAAGAAGACCTGCTACTCCA | |
| | AAGAAGCCTGTGGGCGAAGTTCACAGTCAATTTAGTACAGGCCACGCA | |
| | AACTCTCCTTGTACCATAATAATAGGGAAAGCTCATACTGAAAAAGTAC | |
| | ATGTGCCTGCTCGACCCTACAGAGTGCTCAACAACTTCATTTCCAACCA | |
| | AAAAATGGACTTTAAGGAAGATCTTTCAGGAATAGCTGAAATGTTCAA | |
| | GACCCCAGTGAAGGAGCAACCGCAGTTGACAAGCACATGTCACATCGC | |
| | TATTTCAAATTCAGAGAATTTGCTTGGAAAACAGTTTCAAGGAACTGAT | |
| | TCAGGAGAAGAACCTCTGCTCCCCACCTCAGAGAGTTTTGGAGGAAAT | |
| | GTGTTCTTCAGTGCACAGAATGCAGCAAAACAGCCATCTGATAAATGCT | |
| | CTGCAAGCCCTCCCTTAAGACGGCAGTGTATTAGAGAAAATGGAAACG | |
| | TAGCAAAAACGCCCAGGAACACCTACAAAATGACTTCTCTGGAGACAA | |
| | AAACTTCAGATACTGAGACAGAGCCTTCAAAAACAGTATCCACTGCAA | |
| | ACAGGTCAGGAAGGTCTACAGAGTTCAGGAATATACAGAAGCTACCTG | |
| | TGGAAAGTAAGAGTGAAGAAACAAATACAGAAATTGTTGAGTGCATCC | |
| | TAAAAAGAGGTCAGAAGGCAACACTACTACAACAAAGGAGAGAAGGA | |
| | GAGATGAAGGAAATAGAAAGACCTTTTGAGACATATAAGGAAAATATT | |
| | GAATTAAAAGAAAACGATGAAAAGATGAAAGCAATGAAGAGATCAAG | |
| | AACTTGGGGGCAGAAATGTGCACCAATGTCTGACCTGACAGACCTCAA | |
| | GAGCTTGCCTGATACAGAACTCATGAAAGACACGGCACGTGGCCAGAA | |
| | TCTCCTCCAAACCCAAGATCATGCCAAGGCACCAAAGAGTGAGAAAGG | |
| | CAAAATCACTAAAATGCCCTGCCAGTCATTACAACCAGAACCAATAAA | |
| | CACCCCAACACACACAAAACAACAGTTGAAGGCATCCCTGGGGAAAGT | |
| | AGGTGTGAAAGAAGAGCTCCTAGCAGTCGGCAAGTTCACACGGACGTC | |
| | AGGGGAGACCACGCACACGCACAGAGAGCCAGCAGGAGATGGCAAGA | |
| | GCATCAGAACGTTTAAGGAGTCTCCAAAGCAGATCCTGGACCCAGCAG | |
| | CCCGTGTAACTGGAATGAAGAAGTGGCCAAGAACGCCTAAGGAAGAGG | |
| | CCCAGTCACTAGAAGACCTGGCTGGCTTCAAAGAGCTCTTCCAGACACC | |
| | AGGTCCCTCTGAGGAATCAATGACTGATGAGAAAACTACCAAAATAGC | |
| | CTGCAAATCTCCACCACCAGAATCAGTGGACACTCCAACAAGCACAAA | |
| | GCAATGGCCTAAGAGAAGTCTCAGGAAAGCAGATGTAGAGGAAGAATT | |
| | CTTAGCACTCAGGAAACTAACACCATCAGCAGGGAAAGCCATGCTTAC | |
| | GCCCAAACCAGCAGGAGGTGATGAGAAAGACATTAAAGCATTTATGGG | |
| | AACTCCAGTGCAGAAACTGGACCTGGCAGGAACTTTACCTGGCAGCAA | |
| | AAGACAGCTACAGACTCCTAAGGAAAAGGCCCAGGCTCTAGAAGACCT | |
| | GGCTGGCTTTAAAGAGCTCTTCCAGACTCCTGGTCACACCGAGGAATTA | |
| | GTGGCTGCTGGTAAAACCACTAAAATACCCTGCGACTCTCCACAGTCAG | |
| | ACCCAGTGGACACCCCAACAAGCACAAAGCAACGACCCAAGAGAAGTA | |
| | TCAGGAAAGCAGATGTAGAGGGAGAACTCTTAGCGTGCAGGAATCTAA | |
| | TGCCATCAGCAGGCAAAGCCATGCACACGCCTAAACCATCAGTAGGTG | |
| | AAGAGAAAGACATCATCATATTTGTGGGAACTCCAGTGCAGAAACTGG | |
| | ACCTGACAGAGAACTTAACCGGCAGCAAGAGACGGCCACAAACTCCTA | |
| | AGGAAGAGGCCCAGGCTCTGGAAGACCTGACTGGCTTTAAAGAGCTCT | |
| | TCCAGACCCCTGGTCATACTGAAGAAGCAGTGGCTGCTGGCAAAACTA | |
| | CTAAAATGCCCTGCGAATCTTCTCCACCAGAATCAGCAGACACCCCAAC | |
| | AAGCACAAGAAGGCAGCCCAAGACACCTTTGGAGAAAAGGGACGTAC | |
| | AGAAGGAGCTCTCAGCCCTGAAGAAGCTCACACAGACATCAGGGGAAA | |
| | CCACACACACAGATAAAGTACCAGGAGGTGAGGATAAAAGCATCAACG | |
| | CGTTTAGGGAAACTGCAAAACAGAAACTGGACCCAGCAGCAAGTGTAA | |
| | CTGGTAGCAAGAGGCACCCAAAAACTAAGGAAAAGGCCCAACCCCTAG | |
| | AAGACCTGGCTGGCTTGAAAGAGCTCTTCCAGACACCAGTATGCACTGA | |
| | CAAGCCCACGACTCACGAGAAAACTACCAAAATAGCCTGCAGATCACA | |
| | ACCAGACCCAGTGGACACACCAACAAGCTCCAAGCCACAGTCCAAGAG | |
| | AAGTCTCAGGAAAGTGGACGTAGAAGAAGAATTCTTCGCACTCAGGAA | |
| | ACGAACACCATCAGCAGGCAAAGCCATGCACACACCCAAACCAGCAGT | |
| | AAGTGGTGAGAAAAACATCTACGCATTTATGGGAACTCCAGTGCAGAA | |
| | ACTGGACCTGACAGAGAACTTAACTGGCAGCAAGAGACGGCTACAAAC | |
| | TCCTAAGGAAAAGGCCCAGGCTCTAGAAGACCTGGCTGGCTTTAAAGA | |
| | GCTCTTCCAGACACGAGGTCACACTGAGGAATCAATGACTAACGATAA | |
| | AACTGCCAAAGTAGCCTGCAAATCTTCACAACCAGACCCAGACAAAAA | |
| | CCCAGCAAGCTCCAAGCGACGGCTCAAGACATCCCTGGGGAAAGTGGG | |
| | CGTGAAAGAAGAGCTCCTAGCAGTTGGCAAGCTCACACAGACATCAGG | |
| | AGAGACTACACACACACACACAGAGCCAACAGGAGATGGTAAGAGCAT | |
| | GAAAGCATTTATGGAGTCTCCAAAGCAGATCTTAGACTCAGCAGCAAG | |
| | TCTAACTGGCAGCAAGAGGCAGCTGAGAACTCCTAAGGGAAAGTCTGA | |
| | AGTCCCTGAAGACCTGGCCGGCTTCATCGAGCTCTTCCAGACACCAAGT | |
| | CACACTAAGGAATCAATGACTAACGAAAAAACTACCAAAGTATCCTAC | |
| | AGAGCTTCACAGCCAGACCTAGTGGACACCCCAACAAGCTCCAAGCCA | |
| | CAGCCCAAGAGAAGTCTCAGGAAAGCAGACACTGAAGAAGAATTTTTA | |
| | GCATTTAGGAAACAAACGCCATCAGCAGGCAAAGCCATGCACACACCC | |
| | AAACCAGCAGTAGGTGAAGAGAAAGACATCAACACGTTTTTGGGAACT | |
| | CCAGTGCAGAAACTGGACCAGCCAGGAAATTTACCTGGCAGCAATAGA | |
| | CGGCTACAAACTCGTAAGGAAAAGGCCCAGGCTCTAGAAGAACTGACT | |
| | GGCTTCAGAGAGCTTTTCCAGACACCATGCACTGATAACCCCACGACTG | |
| | ATGAGAAAACTACCAAAAAAATACTCTGCAAATCTCCGCAATCAGACC | |
| | CAGCGGACACCCCAACAAACACAAAGCAACGGCCCAAGAGAAGCCTCA | |
| | AGAAAGCAGACGTAGAGGAAGAATTTTTAGCATTCAGGAAACTAACAC | |
| | CATCAGCAGGCAAAGCCATGCACACGCCTAAAGCAGCAGTAGGTGAAG | |
| | AGAAAGACATCAACACATTTGTGGGGACTCCAGTGGAGAAACTGGACC | |
| | TGCTAGGAAATTTACCTGGCAGCAAGAGACGGCCACAAACTCCTAAAG | |
| | AAAAGGCCAAGGCTCTAGAAGATCTGGCTGGCTTCAAAGAGCTCTTCC | |
| | AGACACCAGGTCACACTGAGGAATCAATGACCGATGACAAAATCACAG | |
| | AAGTATCCTGCAAATCTCCACAACCAGACCCAGTCAAAACCCCAACAA | |
| | GCTCCAAGCAACGACTCAAGATATCCTTGGGGAAAGTAGGTGTGAAAG | |
| | AAGAGGTCCTACCAGTCGGCAAGCTCACACAGACGTCAGGGAAGACCA | |
| | CACAGACACACAGAGAGACAGCAGGAGATGGAAAGAGCATCAAAGCG | |
| | TTTAAGGAATCTGCAAAGCAGATGCTGGACCCAGCAAACTATGGAACT | |
| | GGGATGGAGAGGTGGCCAAGAACACCTAAGGAAGAGGCCCAATCACTA | |
| | GAAGACCTGGCCGGCTTCAAAGAGCTCTTCCAGACACCAGACCACACT | |
| | GAGGAATCAACAACTGATGACAAAACTACCAAAATAGCCTGCAAATCT | |
| | CCACCACCAGAATCAATGGACACTCCAACAAGCACAAGGAGGCGGCCC | |
| | AAAACACCTTTGGGGAAAAGGGATATAGTGGAAGAGCTCTCAGCCCTG | |
| | AAGCAGCTCACACAGACCACACACACAGACAAAGTACCAGGAGATGAG | |
| | GATAAAGGCATCAACGTGTTCAGGGAAACTGCAAAACAGAAACTGGAC | |
| | CCAGCAGCAAGTGTAACTGGTAGCAAGAGGCAGCCAAGAACTCCTAAG | |
| | GGAAAAGCCCAACCCCTAGAAGACTTGGCTGGCTTGAAAGAGCTCTTC | |
| | CAGACACCAATATGCACTGACAAGCCCACGACTCATGAGAAAACTACC | |
| | AAAATAGCCTGCAGATCTCCACAACCAGACCCAGTGGGTACCCCAACA | |
| | ATCTTCAAGCCACAGTCCAAGAGAAGTCTCAGGAAAGCAGACGTAGAG | |
| | GAAGAATCCTTAGCACTCAGGAAACGAACACCATCAGTAGGGAAAGCT | |
| | ATGGACACACCCAAACCAGCAGGAGGTGATGAGAAAGACATGAAAGC | |
| | ATTTATGGGAACTCCAGTGCAGAAATTGGACCTGCCAGGAAATTTACCT | |
| | GGCAGCAAAAGATGGCCACAAACTCCTAAGGAAAAGGCCCAGGCTCTA | |
| | GAAGACCTGGCTGGCTTCAAAGAGCTCTTCCAGACACCAGGCACTGAC | |
| | AAGCCCACGACTGATGAGAAAACTACCAAAATAGCCTGCAAATCTCCA | |
| | CAACCAGACCCAGTGGACACCCCAGCAAGCACAAAGCAACGGCCCAAG | |
| | AGAAACCTCAGGAAAGCAGACGTAGAGGAAGAATTTTTAGCACTCAGG | |
| | AAACGAACACCATCAGCAGGCAAAGCCATGGACACACCAAAACCAGCA | |
| | GTAAGTGATGAGAAAAATATCAACACATTTGTGGAAACTCCAGTGCAG | |
| | AAACTGGACCTGCTAGGAAATTTACCTGGCAGCAAGAGACAGCCACAG | |
| | ACTCCTAAGGAAAAGGCTGAGGCTCTAGAGGACCTGGTTGGCTTCAAA | |
| | GAACTCTTCCAGACACCAGGTCACACTGAGGAATCAATGACTGATGAC | |
| | AAAATCACAGAAGTATCCTGTAAATCTCCACAGCCAGAGTCATTCAAA | |
| | ACCTCAAGAAGCTCCAAGCAAAGGCTCAAGATACCCCTGGTGAAAGTG | |
| | GACATGAAAGAAGAGCCCCTAGCAGTCAGCAAGCTCACACGGACATCA | |
| | GGGGAGACTACGCAAACACACACAGAGCCAACAGGAGATAGTAAGAG | |
| | CATCAAAGCGTTTAAGGAGTCTCCAAAGCAGATCCTGGACCCAGCAGC | |
| | AAGTGTAACTGGTAGCAGGAGGCAGCTGAGAACTCGTAAGGAAAAGGC | |
| | CCGTGCTCTAGAAGACCTGGTTGACTTCAAAGAGCTCTTCTCAGCACCA | |
| | GGTCACACTGAAGAGTCAATGACTATTGACAAAAACACAAAAATTCCC | |
| | TGCAAATCTCCCCCACCAGAACTAACAGACACTGCCACGAGCACAAAG | |
| | AGATGCCCCAAGACACGTCCCAGGAAAGAAGTAAAAGAGGAGCTCTCA | |
| | GCAGTTGAGAGGCTCACGCAAACATCAGGGCAAAGCACACACACACAC | |
| | AAAGAACCAGCAAGCGGTGATGAGGGCATCAAAGTATTGAAGCAACGT | |
| | GCAAAGAAGAAACCAAACCCAGTAGAAGAGGAACCCAGCAGGAGAAG | |
| | GCCAAGAGCACCTAAGGAAAAGGCCCAACCCCTGGAAGACCTGGCCGG | |
| | CTTCACAGAGCTCTCTGAAACATCAGGTCACACTCAGGAATCACTGACT | |
| | GCTGGCAAAGCCACTAAAATACCCTGCGAATCTCCCCCACTAGAAGTG | |
| | GTAGACACCACAGCAAGCACAAAGAGGCATCTCAGGACACGTGTGCAG | |
| | AAGGTACAAGTAAAAGAAGAGCCTTCAGCAGTCAAGTTCACACAAACA | |
| | TCAGGGGAAACCACGGATGCAGACAAAGAACCAGCAGGTGAAGATAA | |
| | AGGCATCAAAGCATTGAAGGAATCTGCAAAACAGACACCGGCTCCAGC | |
| | AGCAAGTGTAACTGGCAGCAGGAGACGGCCAAGAGCACCCAGGGAAA | |
| | GTGCCCAAGCCATAGAAGACCTAGCTGGCTTCAAAGACCCAGCAGCAG | |
| | GTCACACTGAAGAATCAATGACTGATGACAAAACCACTAAAATACCCT | |
| | GCAAATCATCACCAGAACTAGAAGACACCGCAACAAGCTCAAAGAGAC | |
| | GGCCCAGGACACGTGCCCAGAAAGTAGAAGTGAAGGAGGAGCTGTTAG | |
| | CAGTTGGCAAGCTCACACAAACCTCAGGGGAGACCACGCACACCGACA | |
| | AAGAGCCGGTAGGTGAGGGCAAAGGCACGAAAGCATTTAAGCAACCTG | |
| | CAAAGCGGAAGCTGGACGCAGAAGATGTAATTGGCAGCAGGAGACAG | |
| | CCAAGAGCACCTAAGGAAAAGGCCCAACCCCTGGAAGATCTGGCCAGC | |
| | TTCCAAGAGCTCTCTCAAACACCAGGCCACACTGAGGAACTGGCAAAT | |
| | GGTGCTGCTGATAGCTTTACAAGCGCTCCAAAGCAAACACCTGACAGTG | |
| | GAAAACCTCTAAAAATATCCAGAAGAGTTCTTCGGGCCCCTAAAGTAG | |
| | AACCCGTGGGAGACGTGGTAAGCACCAGAGACCCTGTAAAATCACAAA | |
| | GCAAAAGCAACACTTCCCTGCCCCCACTGCCCTTCAAGAGGGGAGGTG | |
| | GCAAAGATGGAAGCGTCACGGGAACCAAGAGGCTGCGCTGCATGCCAG | |
| | CACCAGAGGAAATTGTGGAGGAGCTGCCAGCCAGCAAGAAGCAGAGG | |
| | GTTGCTCCCAGGGCAAGAGGCAAATCATCCGAACCCGTGGTCATCATG | |
| | AAGAGAAGTTTGAGGACTTCTGCAAAAAGAATTGAACCTGCGGAAGAG | |
| | CTGAACAGCAACGACATGAAAACCAACAAAGAGGAACACAAATTACA | |
| | AGACTCGGTCCCTGAAAATAAGGGAATATCCCTGCGCTCCAGACGCCA | |
| | AAATAAGACTGAGGCAGAACAGCAAATAACTGAGGTCTTTGTATTAGC | |
| | AGAAAGAATAGAAATAAACAGAAATGAAAAGAAGCCCATGAAGACCT | |
| | CCCCAGAGATGGACATTCAGAATCCAGATGATGGAGCCCGGAAACCCA | |
| | TACCTAGAGACAAAGTCACTGAGAACAAAAGGTGCTTGAGGTCTGCTA | |
| | GACAGAATGAGAGCTCCCAGCCTAAGGTGGCAGAGGAGAGCGGAGGG | |
| | CAGAAGAGTGCGAAGGTTCTCATGCAGAATCAGAAAGGGAAAGGAGA | |
| | AGCAGGAAATTCAGACTCCATGTGCCTGAGATCAAGAAAGACAAAAAG | |
| | CCAGCCTGCAGCAAGCACTTTGGAGAGCAAATCTGTGCAGAGAGTAAC | |
| | GCGGAGTGTCAAGAGGTGTGCAGAAAATCCAAAGAAGGCTGAGGACA | |
| | ATGTGTGTGTCAAGAAAATAAGAACCAGAAGTCATAGGGACAGTGAAG | |
| | ATATTTGACAGAAAAATCGAACTGGGAAAAATATAATAAAGTTAGTTTT | |
| | GTGATAAGTTCTAGTGCAGTTTTTGTCATAAATTACAAGTGAATTCTGT | |
| | AAGTAAGGCTGTCAGTCTGCTTAAGGGAAGAAAACTTTGGATTTGCTGG | |
| | GTCTGAATCGGCTTCATAAACTCCACTGGGAGCACTGCTGGGCTCCTGG | |
| | ACTGAGAATAGTTGAACACCGGGGGCTTTGTGAAGGAGTCTGGGCCAA | |
| | GGTTTGCCCTCAGCTTTGCAGAATGAAGCCTTGAGGTCTGTCACCACCC | |
| | ACAGCCACCCTACAGCAGCCTTAACTGTGACACTTGCCACACTGTGTCG | |
| | TCGTTTGTTTGCCTATGTCCTCCAGGGCACGGTGGCAGGAACAACTATC | |
| | CTCGTCTGTCCCAACACTGAGCAGGCACTCGGTAAACACGAATGAATG | |
| | GATGAGCGCACGGATGAATGGAGCTTACAAGATCTGTCTTTCCAATGGC | |
| | CGGGGGCATTTGGTCCCCAAATTAAGGCTATTGGACATCTGCACAGGAC | |
| | AGTCCTATTTTTGATGTCCTTTCCTTTCTGAAAATAAAGTTTTGTGCTTT | |
| | GGAGAATGACTCGTGAGCACATCTTTAGGGACCAAGAGTGACTTTCTGT | |
| | AAGGAGTGACTCGTGGCTTGCCTTGGTCTCTTGGGAATACTTTTCTAACT | |
| | AGGGTTGCTCTCACCTGAGACATTCTCCACCCGCGGAATCTCAGGGTCC | |
| | CAGGCTGTGGGCCATCACGACCTCAAACTGGCTCCTAATCTCCAGCTTT | |
| | CCTGTCATTGAAAGCTTCGGAAGTTTACTGGCTCTGCTCCCGCCTGTTTT | |
| | CTTTCTGACTCTATCTGGCAGCCCGATGCCACCCAGTACAGGAAGTGAC | |
| | ACCAGTACTCTGTAAAGCATCATCATCCTTGGAGAGACTGAGCACTCAG | |
| | CACCTTCAGCCACGATTTCAGGATCGCTTCCTTGTGAGCCGCTGCCTCC | |
| | GAAATCTCCTTTGAAGCCCAGACATCTTTCTCCAGCTTCAGACTTGTAG | |
| | ATATAACTCGTTCATCTTCATTTACTTTCCACTTTGCCCCCTGTCCTCTCT | |
| | GTGTTCCCCAAATCAGAGAATAGCCCGCCATCCCCCAGGTCACCTGTCT | |
| | GGATTCCTCCCCATTCACCCACCTTGCCAGGTGCAGGTGAGGATGGTGC | |
| | ACCAGACAGGGTAGCTGTCCCCCAAAATGTGCCCTGTGCGGGCAGTGC | |
| | CCTGTCTCCACGTTTGTTTCCCCAGTGTCTGGCGGGGAGCCAGGTGACA | |
| | TCATAAATACTTGCTGAATGAATGCAGAAATCAGCGGTACTGACTTGTA | |
| | CTATATTGGCTGCCATGATAGGGTTCTCACAGCGTCATCCATGATCGTA | |
| | AGGGAGAATGACATTCTGCTTGAGGGAGGGAATAGAAAGGGGCAGGG | |
| | AGGGGACATCTGAGGGCTTCACAGGGCTGCAAAGGGTACAGGGATTGC | |
| | ACCAGGGCAGAACAGGGGAGGGTGTTCAAGGAAGAGTGGCTCTTAGCA | |
| | GAGGCACTTTGGAAGGTGTGAGGCATAAATGCTTCCTTCTACGTAGGCC | |
| | AACCTCAAAACTTTCAGTAGGAATGTTGCTATGATCAAGTTGTTCTAAC | |
| | ACTTTAGACTTAGTAGTAATTATGAACCTCACATAGAAAAATTTCATCC | |
| | AGCCATATGCCTGTGGAGTGGAATATTCTGTTTAGTAGAAAAATCCTTT | |
| | AGAGTTCAGCTCTAACCAGAAATCTTGCTGAAGTATGTCAGCACCTTTT | |
| | CTCACCCTGGTAAGTACAGTATTTCAAGAGCACGCTAAGGGTGGTTTTC | |
| | ATTTTACAGGGCTGTTGATGATGGGTTAAAAATGTTCATTTAAGGGCTA | |
| | CCCCCGTGTTTAATAGATGAACACCACTTCTACACAACCCTCCTTGGTA | |
| | CTGGGGGAGGGAGAGATCTGACAAATACTGCCCATTCCCCTAGGCTGA | |
| | CTGGATTTGAGAACAAATACCCACCCATTTCCACCATGGTATGGTAACT | |
| | TCTCTGAGCTTCAGTTTCCAAGTGAATTTCCATGTAATAGGACATTCCCA | |
| | TTAAATACAAGCTGTTTTTACTTTTTCGCCTCCCAGGGCCTGTGGGATCT | |
| | GGTCCCCCAGCCTCTCTTGGGCTTTCTTACACTAACTCTGTACCTACCAT | |
| | CTCCTGCCTCCCTTAGGCAGGCACCTCCAACCACCACACACTCCCTGCT | |
| | GTTTTCCCTGCCTGGAACTTTCCCTCCTGCCCCACCAAGATCATTTCATC | |
| | CAGTCCTGAGCTCAGCTTAAGGGAGGCTTCTTGCCTGTGGGTTCCCTCA | |
| | CCCCCATGCCTGTCCTCCAGGCTGGGGCAGGTTCTTAGTTTGCCTGGAA | |
| | TTGTTCTGTACCTCTTTGTAGCACGTAGTGTTGTGGAAACTAAGCCACTA | |
| | ATTGAGTTTCTGGCTCCCCTCCTGGGGTTGTAAGTTTTGTTCATTCATGA | |
| | GGGCCGACTGCATTTCCTGGTTACTCTATCCCAGTGACCAGCCACAGGA | |
| | GATGTCCAATAAAGTATGTGATGAAATGGTCTTAAAAAAAAAAAAAA | |
| NM_024101 | GCGCCGGGACGTGGCCAGTTGCCCGCCTGCCCCGGAGAGCCAGGCGCT | 183 |
| | AACCAGCCGCTCTGCGCCCCGCGCCCTGCTTGCCCCCATTATCCAGCCT | |
| | TGCCCCGGCGCCCTGACCTGACGCCCTGGCCTGACGCCCTGCTTCGTCG | |
| | CCTCCTTTCTCTCCCAGGTGCTGGACCAGGGACTGAGCGTCCCCCGGAG | |
| | AGGGTCCGGTGTGACCCCGACAAGAAGCAGAAATGGGGAAGAAACTG | |
| | GATCTTTCCAAGCTCACTGATGAAGAGGCCCAGCATGTCTTGGAAGTTG | |
| | TTCAACGAGATTTTGACCTCCGAAGGAAAGAAGAGGAACGGCTAGAGG | |
| | CGTTGAAGGGCAAGATTAAGAAGGAAAGCTCCAAGAGGGAGCTGCTTT | |
| | CCGACACTGCCCATCTGAACGAGACCCACTGCGCCCGCTGCCTGCAGCC | |
| | CTACCAGCTGCTTGTGAATAGCAAAAGGCAGTGCCTGGAATGTGGCCTC | |
| | TTCACCTGCAAAAGCTGTGGCCGCGTCCACCCGGAGGAGCAGGGCTGG | |
| | ATCTGTGACCCCTGCCATCTGGCCAGAGTCGTGAAGATCGGCTCACTGG | |
| | AGTGGTACTATGAGCATGTGAAAGCCCGCTTCAAGAGGTTCGGAAGTG | |
| | CCAAGGTCATCCGGTCCCTCCACGGGCGGCTGCAGGGTGGAGCTGGGC | |
| | CTGAACTGATATCTGAAGAGAGAAGTGGAGACAGCGACCAGACAGATG | |
| | AGGATGGAGAACCTGGCTCAGAGGCCCAGGCCCAGGCCCAGCCCTTTG | |
| | GCAGCAAAAAAAAGCGCCTCCTCTCCGTCCACGACTTCGACTTCGAGGG | |
| | AGACTCAGATGACTCCACTCAGCCTCAAGGTCACTCCCTGCACCTGTCC | |
| | TCAGTCCCTGAGGCCAGGGACAGCCCACAGTCCCTCACAGATGAGTCCT | |
| | GCTCAGAGAAGGCAGCCCCTCACAAGGCTGAGGGCCTGGAGGAGGCTG | |
| | ATACTGGGGCCTCTGGGTGCCACTCCCATCCGGAAGAGCAGCCGACCA | |
| | GCATCTCACCTTCCAGACACGGCGCCCTGGCTGAGCTCTGCCCGCCTGG | |
| | AGGCTCCCACAGGATGGCCCTGGGGACTGCTGCTGCACTCGGGTCGAAT | |
| | GTCATCAGGAATGAGCAGCTGCCCCTGCAGTACTTGGCCGATGTGGACA | |
| | CCTCTGATGAGGAAAGCATCCGGGCTCACGTGATGGCCTCCCACCATTC | |
| | CAAGCGGAGAGGCCGGGCGTCTTCTGAGAGTCAGATCTTTGAGCTGAA | |
| | TAAGCATATTTCAGCTGTGGAATGCCTGCTGACCTACCTGGAGAACACA | |
| | GTTGTGCCTCCCTTGGCCAAGGGTCTAGGTGCTGGAGTGCGCACGGAGG | |
| | CCGATGTAGAGGAGGAGGCCCTGAGGAGGAAGCTGGAGGAGCTGACC | |
| | AGCAACGTCAGTGACCAGGAGACCTCGTCCGAGGAGGAGGAAGCCAAG | |
| | GACGAAAAGGCAGAGCCCAACAGGGACAAATCAGTTGGGCCTCTCCCC | |
| | CAGGCGGACCCGGAGGTGGGCACGGCTGCCCATCAAACCAACAGACAG | |
| | GAAAAAAGCCCCCAGGACCCTGGGGACCCCGTCCAGTACAACAGGACC | |
| | ACAGATGAGGAGCTGTCAGAGCTGGAGGACAGAGTGGCAGTGACGGCC | |
| | TCAGAAGTCCAGCAGGCAGAGAGCGAGGTTTCAGACATTGAATCCAGG | |
| | ATTGCAGCCCTGAGGGCCGCAGGGCTCACGGTGAAGCCCTCGGGAAAG | |
| | CCCCGGAGGAAGTCAAACCTCCCGATATTTCTCCCTCGAGTGGCTGGGA | |
| | AACTTGGCAAGAGACCAGAGGACCCAAATGCAGACCCTTCAAGTGAGG | |
| | CCAAGGCAATGGCTGTGCCCTATCTTCTGAGAAGAAAGTTCAGTAATTC | |
| | CCTGAAAAGTCAAGGTAAAGATGATGATTCTTTTGATCGGAAATCAGTG | |
| | TACCGAGGCTCGCTGACACAGAGAAACCCCAACGCGAGGAAAGGAATG | |
| | GCCAGCCACACCTTCGCGAAACCTGTGGTGGCCCACCAGTCCTAACGGG | |
| | ACAGGACAGAGAGACAGAGCAGCCCTGCACTGTTTTCCCTCCACCACA | |
| | GCCATCCTGTCCCTCATTGGCTCTGTGCTTTCCACTATACACAGTCACCG | |
| | TCCCAATGAGAAACAAGAAGGAGCACCCTCCACATGGACTCCCACCTG | |
| | CAAGTGGACAGCGACATTCAGTCCTGCACTGCTCACCTGGGTTTACTGA | |
| | TGACTCCTGGCTGCCCCACCATCCTCTCTGATCTGTGAGAAACAGCTAA | |
| | GCTGCTGTGACTTCCCTTTAGGACAATGTTGTGTAAATCTTTGAAGGAC | |
| | ACACCGAAGACCTTTATACTGTGATCTTTTACCCCTTTCACTCTTGGCTT | |
| | TCTTATGTTGCTTTCATGAATGGAATGGAAAAAAGATGACTCAGTTAAG | |
| | GCACCAGCCATATGTGTATTCTTGATGGTCTATATCGGGGTGTGAGCAG | |
| | ATGTTTGCGTATTTCTTGTGGGTGTGACTGGATATTAGACATCCGGACA | |
| | AGTGACTGAACTAATGATCTGCTGAATAATGAAGGAGGAATAGACACC | |
| | CCAGTCCCCACCCTACGTGCACCCGCTCTGCAAGTTCCCATGTGATCTG | |
| | TAGACCAGGGGAAATTACACTGCGGTCAAGGGCAGAGCCTGCACATGA | |
| | CAGCAAGTGAGCATTTGATAGATGCTCAGATGCTAGTGCAGAGAGCCT | |
| | GCTGGGAGACGAAGAGACAGCAGGCAGAGCTCCAGATGGGCAAGGAA | |
| | GAGGCTTGGTTCTAGCCTGGCTCTGCCCCTCACTGCAGTGGATCCAGTG | |
| | GGGCAGAGGACAGAGGGTCACAACCAATGAGGGATGTCTGCCAAGGAT | |
| | GGGGGTGCAGAGGCCACAGGAGTCAGCTTGCCACTCGCCCATTGGTTA | |
| | CATAGATGATCTCTCAGACAGGCTGGGACTCAGAGTTATTTCCTAGTAT | |
| | CGGTGTGCCCCATCCAGTTTTAAGTGGAGCCCTCCAAGACTCTCCAGAG | |
| | CTGCCTTTGAACATCCTAACAGTAATCACATCTCACCCTCCCTGAGGTTC | |
| | ACTTTAGACAGGACCCAATGGCTGCACTGCCTTTGTCAGAGGGGGTGCT | |
| | GAGAGGAGTGGCTTCTTTTAGAATCAAACAGTAGAGACAAGAGTCAAG | |
| | CCTTGTGTCTTCAAGCATTGACCAAGTTAAGTGTTTCCTTCCCTCTCTCA | |
| | ATAAGACACTTCCAGGAGCTTTCCAATCTCTCACTTAAAACTAAGGTTT | |
| | GAATCTCAAAGTGTTGCTGGGAGGCTGATACTCCTGCAACTTCAGGAGA | |
| | CCTGTGAGCACACATTAGCAGCTGTTTCTCTGACTCCTTGTGGCATCAG | |
| | ATAAAAACGTGGGAGTTTTTCCATATAATTCCCAGCCTTACTTATAAAT | |
| | TCTATTCTTTGAAAAAATTATTCAGGCTAGGTAAGGTGGCTCATACCTA | |
| | TAATCCCAGCCCTTTGAGAGGCCAAGGTGGGAGAATTGCTTGAGGCCA | |
| | GGAGTTTGAGACCTCCTGGGCAACATAGTGAGATCCCATCTCTACAAAA | |
| | AACAAAACAAAAAAATTACCCAAGCATGATGGTATATGCCTGTAGTCG | |
| | TACCTACTTACTTAGGAGGCTGAGGCAGGAGGATCACTTGAGCCCTGGA | |
| | GGTTGGGGCTGCAGTGAGCCATGATCGCATCACTATACTCGAGCCTGGG | |
| | CAACAGAGTGAGACCTTGTCTCTTAAAAAAATTAATAATAAATAAATG | |
| | AAAATAATTCTTCAGAAAAAAAAAAAAAAAA | |
| NM_005940 | AAGCCCAGCAGCCCCGGGGCGGATGGCTCCGGCCGCCTGGCTCCGCAG | 184 |
| | CGCGGCCGCGCGCGCCCTCCTGCCCCCGATGCTGCTGCTGCTGCTCCAG | |
| | CCGCCGCCGCTGCTGGCCCGGGCTCTGCCGCCGGACGCCCACCACCTCC | |
| | ATGCCGAGAGGAGGGGGCCACAGCCCTGGCATGCAGCCCTGCCCAGTA | |
| | GCCCGGCACCTGCCCCTGCCACGCAGGAAGCCCCCCGGCCTGCCAGCA | |
| | GCCTCAGGCCTCCCCGCTGTGGCGTGCCCGACCCATCTGATGGGCTGAG | |
| | TGCCCGCAACCGACAGAAGAGGTTCGTGCTTTCTGGCGGGCGCTGGGA | |
| | GAAGACGGACCTCACCTACAGGATCCTTCGGTTCCCATGGCAGTTGGTG | |
| | CAGGAGCAGGTGCGGCAGACGATGGCAGAGGCCCTAAAGGTATGGAGC | |
| | GATGTGACGCCACTCACCTTTACTGAGGTGCACGAGGGCCGTGCTGACA | |
| | TCATGATCGACTTCGCCAGGTACTGGCATGGGGACGACCTGCCGTTTGA | |
| | TGGGCCTGGGGGCATCCTGGCCCATGCCTTCTTCCCCAAGACTCACCGA | |
| | GAAGGGGATGTCCACTTCGACTATGATGAGACCTGGACTATCGGGGAT | |
| | GACCAGGGCACAGACCTGCTGCAGGTGGCAGCCCATGAATTTGGCCAC | |
| | GTGCTGGGGCTGCAGCACACAACAGCAGCCAAGGCCCTGATGTCCGCC | |
| | TTCTACACCTTTCGCTACCCACTGAGTCTCAGCCCAGATGACTGCAGGG | |
| | GCGTTCAACACCTATATGGCCAGCCCTGGCCCACTGTCACCTCCAGGAC | |
| | CCCAGCCCTGGGCCCCCAGGCTGGGATAGACACCAATGAGATTGCACC | |
| | GCTGGAGCCAGACGCCCCGCCAGATGCCTGTGAGGCCTCCTTTGACGCG | |
| | GTCTCCACCATCCGAGGCGAGCTCTTTTTCTTCAAAGCGGGCTTTGTGTG | |
| | GCGCCTCCGTGGGGGCCAGCTGCAGCCCGGCTACCCAGCATTGGCCTCT | |
| | CGCCACTGGCAGGGACTGCCCAGCCCTGTGGACGCTGCCTTCGAGGATG | |
| | CCCAGGGCCACATTTGGTTCTTCCAAGGTGCTCAGTACTGGGTGTACGA | |
| | CGGTGAAAAGCCAGTCCTGGGCCCCGCACCCCTCACCGAGCTGGGCCT | |
| | GGTGAGGTTCCCGGTCCATGCTGCCTTGGTCTGGGGTCCCGAGAAGAAC | |
| | AAGATCTACTTCTTCCGAGGCAGGGACTACTGGCGTTTCCACCCCAGCA | |
| | CCCGGCGTGTAGACAGTCCCGTGCCCCGCAGGGCCACTGACTGGAGAG | |
| | GGGTGCCCTCTGAGATCGACGCTGCCTTCCAGGATGCTGATGGCTATGC | |
| | CTACTTCCTGCGCGGCCGCCTCTACTGGAAGTTTGACCCTGTGAAGGTG | |
| | AAGGCTCTGGAAGGCTTCCCCCGTCTCGTGGGTCCTGACTTCTTTGGCT | |
| | GTGCCGAGCCTGCCAACACTTTCCTCTGACCATGGCTTGGATGCCCTCA | |
| | GGGGTGCTGACCCCTGCCAGGCCACGAATATCAGGCTAGAGACCCATG | |
| | GCCATCTTTGTGGCTGTGGGCACCAGGCATGGGACTGAGCCCATGTCTC | |
| | CTCAGGGGGATGGGGTGGGGTACAACCACCATGACAACTGCCGGGAGG | |
| | GCCACGCAGGTCGTGGTCACCTGCCAGCGACTGTCTCAGACTGGGCAG | |
| | GGAGGCTTTGGCATGACTTAAGAGGAAGGGCAGTCTTGGGCCCGCTAT | |
| | GCAGGTCCTGGCAAACCTGGCTGCCCTGTCTCCATCCCTGTCCCTCAGG | |
| | GTAGCACCATGGCAGGACTGGGGGAACTGGAGTGTCCTTGCTGTATCCC | |
| | TGTTGTGAGGTTCCTTCCAGGGGCTGGCACTGAAGCAAGGGTGCTGGGG | |
| | CCCCATGGCCTTCAGCCCTGGCTGAGCAACTGGGCTGTAGGGCAGGGCC | |
| | ACTTCCTGAGGTCAGGTCTTGGTAGGTGCCTGCATCTGTCTGCCTTCTGG | |
| | CTGACAATCCTGGAAATCTGTTCTCCAGAATCCAGGCCAAAAAGTTCAC | |
| | AGTCAAATGGGGAGGGGTATTCTTCATGCAGGAGACCCCAGGCCCTGG | |
| | AGGCTGCAACATACCTCAATCCTGTCCCAGGCCGGATCCTCCTGAAGCC | |
| | CTTTTCGCAGCACTGCTATCCTCCAAAGCCATTGTAAATGTGTGTACAG | |
| | TGTGTATAAACCTTCTTCTTCTTTTTTTTTTTTTAAACTGAGGATTGTC | |
| BX647151 | TAGCAGCACACAAGGGTTCGTGTTTGTGGAACCAGGTAGCTTCCTTCAG | 185 |
| | AGCTGACATTTGCCCACAGCCAGCCTGGCCCAGCCCCATACCACCAGCC | |
| | CTGGCGCTCTGGGGCGTGAGGTGCCTTTTCTGCCCCCCTGCTCTAGGGC | |
| | AGGTGGAAATCACCCATGGTGGGTCTACATCTGATAGAAGCATCTTATA | |
| | GTTCTGCTTCTGGACCAGACCATCCTGGGTTTTTCTCTGTTCTGCTGAAG | |
| | GGTTCCCTCCACGTGTCCATCACCTCGGTGAACTCTTGGGAGACCTGGG | |
| | AAGATGCTGGCCTCACCTCTCGCCTCTCCTTTCCCTCATTGTGCTGCCAC | |
| | CATCCTTCTCACACAGGCTCTCCAGGGAGAGCTGGGCAGGATGGGATCT | |
| | TCCTGGGTTCCCACCTTGCTCCGTGCCCCCTCTCACTGTTCCTGAAGTGT | |
| | GGCCACGGACTGCCTTGTTTTCTGGAAAGTCCCAAGTCTGGACCATGAC | |
| | TGAGCAGCATTCTCGGCTATCTGCCACCTGTCTGGGGCTCCTGGCCCCT | |
| | CTTAGACTCCCCTCTCCCTTCTGTTTCCCCCGAGCCCCTGACTTGGACCT | |
| | GCAGGGTGGGGAGAGGGATGGGACGAGAACCTGTGCTGGGGCCAAAG | |
| | GTCGCACTGGGGGAAGGTGGAGCCAGGGCAGCAGAGTGCCTGGCGTCG | |
| | GCCCCTATCCTGTCACTAGTTCCCCCGTTCTGGCCCCTGGCAGGTTTGTA | |
| | ACCCCAGATCAGAAGTACTCCATGGACAACACTCCCCACACGCCAACC | |
| | CCGTTCAAGAACGCCCTGGAGAAGTACGGACCCCTGAAGCCCCTGGTA | |
| | CGTGGTGTGGTCACTGCCGTGGATCTCTGCACAGTGGGATCCCTTCGGT | |
| | TCATCCAACCATGTTCAGTCCACAGGACCCTTCCCTCTGAGGTCTCATTT | |
| | GATTCTTTCTCCTGAGAAGATGCAGAGATCCTGATAATATAAATGGGGA | |
| | AGCTGAGGCTGCTCTTTGTCACTTCCTCCGACTGCTCCTGAGCACCTGA | |
| | GTTTGCAAGCACGCGCCGGCTGGTGCTAGAGACATGGTGGTATCCCGTG | |
| | ACACTCAGCCTCAGGATGGGGGAGACTGATGTGAAATACAAATAACTT | |
| | AAACACTTTCAGGCAAAGATAAGCACTGGGCCTAGTTCAGAGAAGTGG | |
| | CAAATTGCTACTCTGGCCTGTCTCTGACCAACTCCCAGTTCTCTACAGA | |
| | GCACGGGAAAGCCCCTCGGGGACGTCTTTCCTGCAGTGTGCAGGCTGCC | |
| | CTTCTCCCCTGCTCTTCCCAGTTGATGGGATGGTTGTGTTTTCTCTATGA | |
| | AAAAAGGAGTTGGCACCTTGGGCTTTCTGAAACACACAGGTGTTTTAGA | |
| | AATCAGTGGAGGGTGAGAGAAAGGCATGGTTGTGGAGGCACTGGACTG | |
| | TGAACAAGGTCTGCAGCGGGTCCCCCTGCTGTCTCTCTCTACTGCATGG | |
| | AGCCTCCTATGAAGCCCAAGGTGGCTGGGGGCTGAGGCTCCCTTGGGCC | |
| | TGCCATGGAACTGATTCTGAGTCAAGCAGACTTTCCACGGACCATGCTA | |
| | CATGAGCCGAGGTGAGGCACTAGTTAGTGCTCCTTTCCTGTTGCAGTGG | |
| | AGATTTGGCTCCTCTGTACTAAAATATCTGCATGCTCTCCAAACAGGTG | |
| | TGAGGGCAAATCACATGACCTTGGCAGCTGTAATTAAAGTTTGTGGGGG | |
| | CTTTTCGGATGACTTATGAGGAGTGGCTGTGATTCGCACCTTTCACTCTT | |
| | AGTAGCACTCGCCCTCCCCTGTTCTCTGTTGCCTGAAGCTGGAGAGGTC | |
| | CTTGGAACCCCGAGGCCTGAGAAAGGGAAATGGGTTTGAGAGCCCCCA | |
| | TTAGTGTGGAACAAAGGGTTGAGTGAGCCTGGGCTTTGAGCTGTCGGG | |
| | GTCCTAATTCAGCAGCTGTGTGACTGTGTGCCAGGCTGTTGATCTCTGA | |
| | GCTTCTGTTTCTACCTGCTTAAAATGACGGTTACTGCACAGGGCTGTGT | |
| | GAGGGTTACAGTGCGTCTCTGGGCTGCTCCCAGCCATGGCAGGCCCCTG | |
| | GGAATCAAGGTCATCAGCTGCTTGTCCAAGGCAGCAGTTAGTGGTTGTG | |
| | AATGGTGCGTGTGAGATCTGCATCCTGGCGTCAGGCCTCCTTCCTGCCT | |
| | TACCCAGGACAGCCCAGTTGCAGCTGGGTTGGTCCCACAGTCCCACACA | |
| | CACACAGCCCGAGTGTGGTGCCTCACGTGGGCTGCCCCGTGCCTACCCA | |
| | CAGCCACAGACCCCGCACCTGGAGGAGGACTTGAAGGAGGTGCTGCGT | |
| | TCTGAGGCTGGCATCGAACTCATCATCGAGGACGACATCAGGCCCGAG | |
| | AAGCAGAAGAGGAAGCCTGGGCTGCGGCGGAGCCCCATCAAGAAAGTC | |
| | CGGAAGTCTCTGGCTCTTGACATTGTGGATGAGGATGTGAAGCTGATGA | |
| | TGTCCACACTGCCCAAGTCTCTATCCTTGCCGACAACTGCCCCTTCAAA | |
| | CTCTTCCAGCCTCACCCTGTCAGGTATCAAAGAAGACAACAGCTTGCTC | |
| | AACCAGGGCTTCTTGCAGGCCAAGCCCGAGAAGGCAGCAGTGGCCCAG | |
| | AAGCCCCGAAGCCACTTCACGACACCTGCCCCTATGTCCAGTGCCTGGA | |
| | AGACGGTGGCCTGCGGGGGGACCAGGGACCAGCTTTTCATGCAGGAGA | |
| | AAGCCCGGCAGCTCCTGGGCCGCCTGAAGCCCAGCCACACATCTCGGA | |
| | CCCTCATCTTGTCCTGAGGTGTTGAGGGTGTCACGAGCCCATTCACATG | |
| | TTTACAGGGGTTGTGGGGGCAGAGGGGGTCTGTGAATCTGAGAGTCATT | |
| | CAGGTGACCTCCTGCAGGGAGCCTTCTGCCACCAGCCCCTCCCCAGACT | |
| | CTCAGGTGGAGGCAACAGGGCCATGTGCTGCCCTGTTGCCGAGCCCAG | |
| | CTGTGGGCGGCTCCTGGTGCTAACAACAAAGTTCCACTTCCAGGTCTGC | |
| | CTGGTTCCCCCCCCAAGGCCACAGGGAGCTCCGTCAGCTTCTCCCAAGC | |
| | CCACGTCAGGCCTGGCCTCATCTCAGACCCTGCTTAGGATGGGGGATGT | |
| | GGCCAGGGGTGCTCCTGTGCTCACCCTCTCTTGGTGCATTTTTTTGGAAG | |
| | AATAAAATTGCCTCTCTCTTTGAAAAAAAAAAAAAAAAA | |
| NM_002467 | GACCCCCGAGCTGTGCTGCTCGCGGCCGCCACCGCCGGGCCCCGGCCGT | 186 |
| | CCCTGGCTCCCCTCCTGCCTCGAGAAGGGCAGGGCTTCTCAGAGGCTTG | |
| | GCGGGAAAAAGAACGGAGGGAGGGATCGCGCTGAGTATAAAAGCCGG | |
| | TTTTCGGGGCTTTATCTAACTCGCTGTAGTAATTCCAGCGAGAGGCAGA | |
| | GGGAGCGAGCGGGCGGCCGGCTAGGGTGGAAGAGCCGGGCGAGCAGA | |
| | GCTGCGCTGCGGGCGTCCTGGGAAGGGAGATCCGGAGCGAATAGGGGG | |
| | CTTCGCCTCTGGCCCAGCCCTCCCGCTGATCCCCCAGCCAGCGGTCCGC | |
| | AACCCTTGCCGCATCCACGAAACTTTGCCCATAGCAGCGGGCGGGCACT | |
| | TTGCACTGGAACTTACAACACCCGAGCAAGGACGCGACTCTCCCGACG | |
| | CGGGGAGGCTATTCTGCCCATTTGGGGACACTTCCCCGCCGCTGCCAGG | |
| | ACCCGCTTCTCTGAAAGGCTCTCCTTGCAGCTGCTTAGACGCTGGATTTT | |
| | TTTCGGGTAGTGGAAAACCAGCAGCCTCCCGCGACGATGCCCCTCAACG | |
| | TTAGCTTCACCAACAGGAACTATGACCTCGACTACGACTCGGTGCAGCC | |
| | GTATTTCTACTGCGACGAGGAGGAGAACTTCTACCAGCAGCAGCAGCA | |
| | GAGCGAGCTGCAGCCCCCGGCGCCCAGCGAGGATATCTGGAAGAAATT | |
| | CGAGCTGCTGCCCACCCCGCCCCTGTCCCCTAGCCGCCGCTCCGGGCTC | |
| | TGCTCGCCCTCCTACGTTGCGGTCACACCCTTCTCCCTTCGGGGAGACA | |
| | ACGACGGCGGTGGCGGGAGCTTCTCCACGGCCGACCAGCTGGAGATGG | |
| | TGACCGAGCTGCTGGGAGGAGACATGGTGAACCAGAGTTTCATCTGCG | |
| | ACCCGGACGACGAGACCTTCATCAAAAACATCATCATCCAGGACTGTAT | |
| | GTGGAGCGGCTTCTCGGCCGCCGCCAAGCTCGTCTCAGAGAAGCTGGCC | |
| | TCCTACCAGGCTGCGCGCAAAGACAGCGGCAGCCCGAACCCCGCCCGC | |
| | GGCCACAGCGTCTGCTCCACCTCCAGCTTGTACCTGCAGGATCTGAGCG | |
| | CCGCCGCCTCAGAGTGCATCGACCCCTCGGTGGTCTTCCCCTACCCTCTC | |
| | AACGACAGCAGCTCGCCCAAGTCCTGCGCCTCGCAAGACTCCAGCGCCT | |
| | TCTCTCCGTCCTCGGATTCTCTGCTCTCCTCGACGGAGTCCTCCCCGCAG | |
| | GGCAGCCCCGAGCCCCTGGTGCTCCATGAGGAGACACCGCCCACCACC | |
| | AGCAGCGACTCTGAGGAGGAACAAGAAGATGAGGAAGAAATCGATGTT | |
| | GTTTCTGTGGAAAAGAGGCAGGCTCCTGGCAAAAGGTCAGAGTCTGGA | |
| | TCACCTTCTGCTGGAGGCCACAGCAAACCTCCTCACAGCCCACTGGTCC | |
| | TCAAGAGGTGCCACGTCTCCACACATCAGCACAACTACGCAGCGCCTCC | |
| | CTCCACTCGGAAGGACTATCCTGCTGCCAAGAGGGTCAAGTTGGACAGT | |
| | GTCAGAGTCCTGAGACAGATCAGCAACAACCGAAAATGCACCAGCCCC | |
| | AGGTCCTCGGACACCGAGGAGAATGTCAAGAGGCGAACACACAACGTC | |
| | TTGGAGCGCCAGAGGAGGAACGAGCTAAAACGGAGCTTTTTTGCCCTG | |
| | CGTGACCAGATCCCGGAGTTGGAAAACAATGAAAAGGCCCCCAAGGTA | |
| | GTTATCCTTAAAAAAGCCACAGCATACATCCTGTCCGTCCAAGCAGAGG | |
| | AGCAAAAGCTCATTTCTGAAGAGGACTTGTTGCGGAAACGACGAGAAC | |
| | AGTTGAAACACAAACTTGAACAGCTACGGAACTCTTGTGCGTAAGGAA | |
| | AAGTAAGGAAAACGATTCCTTCTAACAGAAATGTCCTGAGCAATCACCT | |
| | ATGAACTTGTTTCAAATGCATGATCAAATGCAACCTCACAACCTTGGCT | |
| | GAGTCTTGAGACTGAAAGATTTAGCCATAATGTAAACTGCCTCAAATTG | |
| | GACTTTGGGCATAAAAGAACTTTTTTATGCTTACCATCTTTTTTTTTTCTT | |
| | TAACAGATTTGTATTTAAGAATTGTTTTTAAAAAATTTTAAGATTTACAC | |
| | AATGTTTCTCTGTAAATATTGCCATTAAATGTAAATAACTTTAATAAAA | |
| | CGTTTATAGCAGTTACACAGAATTTCAATCCTAGTATATAGTACCTAGT | |
| | ATTATAGGTACTATAAACCCTAATTTTTTTTATTTAAGTACATTTTGCTT | |
| | TTTAAAGTTGATTTTTTTCTATTGTTTTTAGAAAAAATAAAATAACTGGC | |
| | AAATATATCATTGAGCCAAATCTTAAAAAAAAAAAAAAA | |
| BC013732 | GTGGGAGGATTGCATTCAGTCTAGTTCCTGGTTGCCGGCTGAAATAACC | 187 |
| | TGCTCTCCAAAATGTCCACAAAAGTGACTTAAGTCAGGTTCCCCCAAAC | |
| | CAGACACCAAGACAAGAATCCATGTGTGTGTGACTGAAGGAAGTGCTG | |
| | GGAGAGCCCCAGCTGCAGCCTGGATGTGAACTGCAACTCCAAAGTGTG | |
| | TCCAGACTCAAGGCAAGGGCACTAGGCTTTCCAGACCTCCTACTAAGTC | |
| | ATTGATCCAGCACTGCCCTGCCAGGACATAAATCCCTGGCACCTCTTGC | |
| | TCTCTGCAAAGGAGGGCAAAGCAGCTTCAGGAGCCCTTGGGAGTCCTC | |
| | CAAAGAGAGTCTAGGGTACAGGTCCGAAAGTAGAAGAACACAGAAGG | |
| | CAGGCCAGGGGCACTGTGAGATGGTAAAAGAGATCTGAAGGGATCCAG | |
| | AATTCAAGCCAGGAAGAAGCAGCAATCTGTCTTCTGGATTAAAACTGA | |
| | AGATCAACCTACTTTCAACTTACTAAGAAAGGGGATCATGGACATTGAA | |
| | GCATATCTTGAAAGAATTGGCTATAAGAAGTCTAGGAACAAATTGGAC | |
| | TTGGAAACATTAACTGATATTCTTCAACACCAGATCCGAGCTGTTCCCT | |
| | TTGAGAACCTTAACATCCATTGTGGGGATGCCATGGACTTAGGCTTAGA | |
| | GGCCATTTTTGATCAAGTTGTGAGAAGAAATCGGGGTGGATGGTGTCTC | |
| | CAGGTCAATCATCTTCTGTACTGGGCTCTGACCACTATTGGTTTTGAGAC | |
| | CACGATGTTGGGAGGGTATGTTTACAGCACTCCAGCCAAAAAATACAG | |
| | CACTGGCATGATTCACCTTCTCCTGCAGGTGACCATTGATGGCAGGAAC | |
| | TACATTGTCGATGCTGGGTTTGGACGCTCATACCAGATGTGGCAGCCTC | |
| | TGGAGTTAATTTCTGGGAAGGATCAGCCTCAGGTGCCTTGTGTCTTCCG | |
| | TTTGACGGAAGAGAATGGATTCTGGTATCTAGACCAAATCAGAAGGGA | |
| | ACAGTACATTCCAAATGAAGAATTTCTTCATTCTGATCTCCTAGAAGAC | |
| | AGCAAATACCGAAAAATCTACTCCTTTACTCTTAAGCCTCGAACAATTG | |
| | AAGATTTTGAGTCTATGAATACATACCTGCAGACATCTCCATCATCTGT | |
| | GTTTACTAGTAAATCATTTTGTTCCTTGCAGACCCCAGATGGGGTTCACT | |
| | GTTTGGTGGGCTTCACCCTCACCCATAGGAGATTCAATTATAAGGACAA | |
| | TACAGATCTAATAGAGTTCAAGACTCTGAGTGAGGAAGAAATAGAAAA | |
| | AGTGCTGAAAAATATATTTAATATTTCCTTGCAGAGAAAGCTTGTGCCC | |
| | AAACATGGTGATAGATTTTTTACTATTTAGAATAAGGAGTAAAACAATC | |
| | TTGTCTATTTGTCATCCAGCTCACCAGTTATCAACTGACGACCTATCATG | |
| | TATCTTCTGTACCCTTACCTTATTTTGAAGAAAATCCTAGACATCAAATC | |
| | ATTTCACCTATAAAAATGTCATCATATATAATTAAACAGCTTTTTAAAG | |
| | AAACATAACCACAAACCTTTTCAAATAATAATAATAATAATAATAATAA | |
| | ATGTCTTTTAAAGATGGCCTGTGGTTATCTTGGAAATTGGTGATTTATGC | |
| | TAGAAAGCTTTTAATGTTGGTTTATTGTTGAATTCCTAGAAAAGTTTTAT | |
| | GGGTAGATGAGTAAATAAAATATTGTAAAAAAACTTATTGTCTATAAA | |
| | GTATATTAAAACATTGTTGGCTAATATAAAAAAAAAAAAAA | |
| NM_014321 | GCGCGCGGGTTTCGTTGACCCGCGGCGTTCACGGGAATTGTTCGCTTTA | 188 |
| | GTGCCGGCGCCATGGGGTCGGAGCTGATCGGGCGCCTAGCCCCGCGCC | |
| | TGGGCCTCGCCGAGCCCGACATGCTGAGGAAAGCAGAGGAGTACTTGC | |
| | GCCTGTCCCGGGTGAAGTGTGTCGGCCTCTCCGCACGCACCACGGAGAC | |
| | CAGCAGTGCAGTCATGTGCCTGGACCTTGCAGCTTCCTGGATGAAGTGC | |
| | CCCTTGGACAGGGCTTATTTAATTAAACTTTCTGGTTTGAACAAGGAGA | |
| | CATATCAGAGCTGTCTTAAATCTTTTGAGTGTTTACTGGGCCTGAATTCA | |
| | AATATTGGAATAAGAGACCTAGCTGTACAGTTTAGCTGTATAGAAGCA | |
| | GTGAACATGGCTTCAAAGATACTAAAAAGCTATGAGTCCAGTCTTCCCC | |
| | AGACACAGCAAGTGGATCTTGACTTATCCAGGCCACTTTTCACTTCTGC | |
| | TGCACTGCTTTCAGCATGCAAGATTCTAAAGCTGAAAGTGGATAAAAAC | |
| | AAAATGGTAGCCACATCCGGTGTAAAAAAAGCTATATTTGATCGACTGT | |
| | GTAAACAACTAGAGAAGATTGGACAGCAGGTCGACAGAGAACCTGGAG | |
| | ATGTAGCTACTCCACCACGGAAGAGAAAGAAGATAGTGGTTGAAGCCC | |
| | CAGCAAAGGAAATGGAGAAGGTAGAGGAGATGCCACATAAACCACAG | |
| | AAAGATGAAGATCTGACACAGGATTATGAAGAATGGAAAAGAAAAATT | |
| | TTGGAAAATGCTGCCAGTGCTCAAAAGGCTACAGCAGAGTGATTTCAG | |
| | CTTCCAAACTGGTATACATTCCAAACTGATAGTACATTGCCATCTCCAG | |
| | GAAGACTTGACGGCTTTGGGATTTTGTTTAAACTTTTATAATAAGGATC | |
| | CTAAGACTGTTGCCTTTAAATAGCAAAGCAGCCTACCTGGAGGCTAAGT | |
| | CTGGGCAGTGGGCTGGCCCCTGGTGTGAGCATTAGACCAGCCACAGTG | |
| | CCTGATTGGTATAGCCTTATGTGCTTTCCTACAAAATGGAATTGGAGGC | |
| | CGGGCGCAGTGGCTCACGCCTGTAATCCCAGCACTTTGGGAGGCCAAG | |
| | GTGGGTGGATCACCTGAGGTCAGGAGCTCGAGACCAGCCTGGCCAACA | |
| | TGGTGAAACCCCATCTCTACTAAAAATACAAAAATTAGCCAGGTGTGAT | |
| | GGTGCATGCCTGTAATCCCAGCTCCTCAGTAGGCTGAGACAGGAGCATC | |
| | ACTTGAACGTGGGAGGCAGAGGTTGCAGTGAGCCGAGATTGCACCACC | |
| | GCACTCCAGCCTGGGTGACAGAGCGAGACTTATCTCATAAATAAATAG | |
| | ATAGATACTCCAGCCTGGGTGACAGAGCGAGACTTATAGATAGATAGA | |
| | TAGATAGATGGATAGATAGATAGATAGATAGATAGATAGATAAACGGA | |
| | ATTGGAGCCATTTTGCTTTAAGTGAATGGCAGTCCCTTGTCTTATTCAGA | |
| | ATATAAAATTCAGTCTGAATGGCATCTTACAGATTTTACTTCAATTTTTG | |
| | TGTACGGTATTTTTTATTTGACTAAATCAATATATTGTACAGCCTAAGTT | |
| | AATAAATGTTATTTATATATGCAAAAAAAAAAAAAAAAA | |
| NM_000926 | AGTCCACAGCTGTCACTAATCGGGGTAAGCCTTGTTGTATTTGTGCGTG | 189 |
| | TGGGTGGCATTCTCAATGAGAACTAGCTTCACTTGTCATTTGAGTGAAA | |
| | TCTACAACCCGAGGCGGCTAGTGCTCCCGCACTACTGGGATCTGAGATC | |
| | TTCGGAGATGACTGTCGCCCGCAGTACGGAGCCAGCAGAAGTCCGACC | |
| | CTTCCTGGGAATGGGCTGTACCGAGAGGTCCGACTAGCCCCAGGGTTTT | |
| | AGTGAGGGGGCAGTGGAACTCAGCGAGGGACTGAGAGCTTCACAGCAT | |
| | GCACGAGTTTGATGCCAGAGAAAAAGTCGGGAGATAAAGGAGCCGCGT | |
| | GTCACTAAATTGCCGTCGCAGCCGCAGCCACTCAAGTGCCGGACTTGTG | |
| | AGTACTCTGCGTCTCCAGTCCTCGGACAGAAGTTGGAGAACTCTCTTGG | |
| | AGAACTCCCCGAGTTAGGAGACGAGATCTCCTAACAATTACTACTTTTT | |
| | CTTGCGCTCCCCACTTGCCGCTCGCTGGGACAAACGACAGCCACAGTTC | |
| | CCCTGACGACAGGATGGAGGCCAAGGGCAGGAGCTGACCAGCGCCGCC | |
| | CTCCCCCGCCCCCGACCCAGGAGGTGGAGATCCCTCCGGTCCAGCCACA | |
| | TTCAACACCCACTTTCTCCTCCCTCTGCCCCTATATTCCCGAAACCCCCT | |
| | CCTCCTTCCCTTTTCCCTCCTCCTGGAGACGGGGGAGGAGAAAAGGGGA | |
| | GTCCAGTCGTCATGACTGAGCTGAAGGCAAAGGGTCCCCGGGCTCCCC | |
| | ACGTGGCGGGCGGCCCGCCCTCCCCCGAGGTCGGATCCCCACTGCTGTG | |
| | TCGCCCAGCCGCAGGTCCGTTCCCGGGGAGCCAGACCTCGGACACCTTG | |
| | CCTGAAGTTTCGGCCATACCTATCTCCCTGGACGGGCTACTCTTCCCTCG | |
| | GCCCTGCCAGGGACAGGACCCCTCCGACGAAAAGACGCAGGACCAGCA | |
| | GTCGCTGTCGGACGTGGAGGGCGCATATTCCAGAGCTGAAGCTACAAG | |
| | GGGTGCTGGAGGCAGCAGTTCTAGTCCCCCAGAAAAGGACAGCGGACT | |
| | GCTGGACAGTGTCTTGGACACTCTGTTGGCGCCCTCAGGTCCCGGGCAG | |
| | AGCCAACCCAGCCCTCCCGCCTGCGAGGTCACCAGCTCTTGGTGCCTGT | |
| | TTGGCCCCGAACTTCCCGAAGATCCACCGGCTGCCCCCGCCACCCAGCG | |
| | GGTGTTGTCCCCGCTCATGAGCCGGTCCGGGTGCAAGGTTGGAGACAGC | |
| | TCCGGGACGGCAGCTGCCCATAAAGTGCTGCCCCGGGGCCTGTCACCA | |
| | GCCCGGCAGCTGCTGCTCCCGGCCTCTGAGAGCCCTCACTGGTCCGGGG | |
| | CCCCAGTGAAGCCGTCTCCGCAGGCCGCTGCGGTGGAGGTTGAGGAGG | |
| | AGGATGGCTCTGAGTCCGAGGAGTCTGCGGGTCCGCTTCTGAAGGGCA | |
| | AACCTCGGGCTCTGGGTGGCGCGGCGGCTGGAGGAGGAGCCGCGGCTG | |
| | TCCCGCCGGGGGCGGCAGCAGGAGGCGTCGCCCTGGTCCCCAAGGAAG | |
| | ATTCCCGCTTCTCAGCGCCCAGGGTCGCCCTGGTGGAGCAGGACGCGCC | |
| | GATGGCGCCCGGGCGCTCCCCGCTGGCCACCACGGTGATGGATTTCATC | |
| | CACGTGCCTATCCTGCCTCTCAATCACGCCTTATTGGCAGCCCGCACTC | |
| | GGCAGCTGCTGGAAGACGAAAGTTACGACGGCGGGGCCGGGGCTGCCA | |
| | GCGCCTTTGCCCCGCCGCGGAGTTCACCCTGTGCCTCGTCCACCCCGGT | |
| | CGCTGTAGGCGACTTCCCCGACTGCGCGTACCCGCCCGACGCCGAGCCC | |
| | AAGGACGACGCGTACCCTCTCTATAGCGACTTCCAGCCGCCCGCTCTAA | |
| | AGATAAAGGAGGAGGAGGAAGGCGCGGAGGCCTCCGCGCGCTCCCCGC | |
| | GTTCCTACCTTGTGGCCGGTGCCAACCCCGCAGCCTTCCCGGATTTCCC | |
| | GTTGGGGCCACCGCCCCCGCTGCCGCCGCGAGCGACCCCATCCAGACCC | |
| | GGGGAAGCGGCGGTGACGGCCGCACCCGCCAGTGCCTCAGTCTCGTCT | |
| | GCGTCCTCCTCGGGGTCGACCCTGGAGTGCATCCTGTACAAAGCGGAGG | |
| | GCGCGCCGCCCCAGCAGGGCCCGTTCGCGCCGCCGCCCTGCAAGGCGC | |
| | CGGGCGCGAGCGGCTGCCTGCTCCCGCGGGACGGCCTGCCCTCCACCTC | |
| | CGCCTCTGCCGCCGCCGCCGGGGCGGCCCCCGCGCTCTACCCTGCACTC | |
| | GGCCTCAACGGGCTCCCGCAGCTCGGCTACCAGGCCGCCGTGCTCAAG | |
| | GAGGGCCTGCCGCAGGTCTACCCGCCCTATCTCAACTACCTGAGGCCGG | |
| | ATTCAGAAGCCAGCCAGAGCCCACAATACAGCTTCGAGTCATTACCTCA | |
| | GAAGATTTGTTTAATCTGTGGGGATGAAGCATCAGGCTGTCATTATGGT | |
| | GTCCTTACCTGTGGGAGCTGTAAGGTCTTCTTTAAGAGGGCAATGGAAG | |
| | GGCAGCACAACTACTTATGTGCTGGAAGAAATGACTGCATCGTTGATAA | |
| | AATCCGCAGAAAAAACTGCCCAGCATGTCGCCTTAGAAAGTGCTGTCA | |
| | GGCTGGCATGGTCCTTGGAGGTCGAAAATTTAAAAAGTTCAATAAAGTC | |
| | AGAGTTGTGAGAGCACTGGATGCTGTTGCTCTCCCACAGCCAGTGGGCG | |
| | TTCCAAATGAAAGCCAAGCCCTAAGCCAGAGATTCACTTTTTCACCAGG | |
| | TCAAGACATACAGTTGATTCCACCACTGATCAACCTGTTAATGAGCATT | |
| | GAACCAGATGTGATCTATGCAGGACATGACAACACAAAACCTGACACC | |
| | TCCAGTTCTTTGCTGACAAGTCTTAATCAACTAGGCGAGAGGCAACTTC | |
| | TTTCAGTAGTCAAGTGGTCTAAATCATTGCCAGGTTTTCGAAACTTACA | |
| | TATTGATGACCAGATAACTCTCATTCAGTATTCTTGGATGAGCTTAATG | |
| | GTGTTTGGTCTAGGATGGAGATCCTACAAACACGTCAGTGGGCAGATGC | |
| | TGTATTTTGCACCTGATCTAATACTAAATGAACAGCGGATGAAAGAATC | |
| | ATCATTCTATTCATTATGCCTTACCATGTGGCAGATCCCACAGGAGTTTG | |
| | TCAAGCTTCAAGTTAGCCAAGAAGAGTTCCTCTGTATGAAAGTATTGTT | |
| | ACTTCTTAATACAATTCCTTTGGAAGGGCTACGAAGTCAAACCCAGTTT | |
| | GAGGAGATGAGGTCAAGCTACATTAGAGAGCTCATCAAGGCAATTGGT | |
| | TTGAGGCAAAAAGGAGTTGTGTCGAGCTCACAGCGTTTCTATCAACTTA | |
| | CAAAACTTCTTGATAACTTGCATGATCTTGTCAAACAACTTCATCTGTAC | |
| | TGCTTGAATACATTTATCCAGTCCCGGGCACTGAGTGTTGAATTTCCAG | |
| | AAATGATGTCTGAAGTTATTGCTGCACAATTACCCAAGATATTGGCAGG | |
| | GATGGTGAAACCCCTTCTCTTTCATAAAAAGTGAATGTCATCTTTTTCTT | |
| | TTAAAGAATTAAATTTTGTGGTATGTCTTTTTGTTTTGGTCAGGATTATG | |
| | AGGTCTTGAGTTTTTATAATGTTCTTCTGAAAGCCTTACATTTATAACAT | |
| | CATAGTGTGTAAATTTAAAAGAAAAATTGTGAGGTTCTAATTATTTTCT | |
| | TTTATAAAGTATAATTAGAATGTTTAACTGTTTTGTTTACCCATATTTTC | |
| | TTGAAGAATTTACAAGATTGAAAAAGTACTAAAATTGTTAAAGTAAACT | |
| | ATCTTATCCATATTATTTCATACCATGTAGGTGAGGATTTTTAACTTTTG | |
| | CATCTAACAAATCATCGACTTAAGAGAAAAAATCTTACATGTAATAACA | |
| | CAAAGCTATTATATGTTATTTCTAGGTAACTCCCTTTGTGTCAATTATAT | |
| | TTCCAAAAATGAACCTTTAAAATGGTATGCAAAATTTTGTCTATATATA | |
| | TTTGTGTGAGGAGGAAATTCATAACTTTCCTCAGATTTTCAAAAGTATTT | |
| | TTAATGCAAAAAATGTAGAAAGAGTTTAAAACCACTAAAATAGATTGA | |
| | TGTTCTTCAAACTAGGCAAAACAACTCATATGTTAAGACCATTTTCCAG | |
| | ATTGGAAACACAAATCTCTTAGGAAGTTAATAAGTAGATTCATATCATT | |
| | ATGCAAATAGTATTGTGGGTTTTGTAGGTTTTTAAAATAACCTTTTTTGG | |
| | GGAGAGAATTGTCCTCTAATGAGGTATTGCGAGTGGACATAAGAAATC | |
| | AGAAGATTATGGCCTAACTGTACTCCTTACCAACTGTGGCATGCTGAAA | |
| | GTTAGTCACTCTTACTGATTCTCAATTCTCTCACCTTTGAAAGTAGTAAA | |
| | ATATCTTTCCTGCCAATTGCTCCTTTGGGTCAGAGCTTATTAACATCTTT | |
| | TCAAATCAAAGGAAAGAAGAAAGGGAGAGGAGGAGGAGGGAGGTATC | |
| | AATTCACATACCTTTCTCCTCTTTATCCTCCACTATCATGAATTCATATT | |
| | ATGTTTCAGCCATGCAAATCTTTTTACCATGAAATTTCTTCCAGAATTTT | |
| | CCCCCTTTGACACAAATTCCATGCATGTTTCAACCTTCGAGACTCAGCC | |
| | AAATGTCATTTCTGTAAAATCTTCCCTGAGTCTTCCAAGCAGTAATTTGC | |
| | CTTCTCCTAGAGTTTACCTGCCATTTTGTGCACATTTGAGTTACAGTAGC | |
| | ATGTTATTTTACAATTGTGACTCTCCTGGGAGTCTGGGAGCCATATAAA | |
| | GTGGTCAATAGTGTTTGCTGACTGAGAGTTGAATGACATTTTCTCTCTGT | |
| | CTTGGTATTACTGTAGATTTCGATCATTCTTTGGTTACATTTCTGCATAT | |
| | TTCTGTACCCATGACTTTATCACTTTCTTCTCCCATGCTTTATCTCCATCA | |
| | ATTATCTTCATTACTTTTAAATTTTCCACCTTTGCTTCCTACTTTGTGAGA | |
| | TCTCTCCCTTTACTGACTATAACATAGAAGAATAGAAGTGTATTTTATGT | |
| | GTCTTAAGGACAATACTTTAGATTCCTTGTTCTAAGTTTTTAAACTGAAT | |
| | GAATGGAATATTATTTCTCTCCCTAAGCAAAATTCCACAAAACAATTAT | |
| | TTCTTATGTTTATGTAGCCTTAAATTGTTTTGTACTGTAAACCTCAGCAT | |
| | AAAAACTTTCTTCATTTCTAATTTCATTCAACAAATATTGATTGAATACC | |
| | TGGTATTAGCACAAGAAAAATGTGCTAATAAGCCTTATGAGAATTTGGA | |
| | GCTGAAGAAAGACATATAACTCAGGAAAGTTACAGTCCAGTAGTAGGT | |
| | ATAAATTACAGTGCCTGATAAATAGGCATTTTAATATTTGTACACTCAA | |
| | CGTATACTAGGTAGGTGCAAAACATTTACATATAATTTTACTGATACCC | |
| | ATGCAGCACAAAGGTACTAACTTTAAATATTAAATAACACCTTTATGTG | |
| | TCAGTAATTCATTTGCATTAAATCTTATTGAAAAGGCTTTCAATATATTT | |
| | TCCCCACAAATGTCATCCCAAGAAAAAAGTATTTTTAACATCTCCCAAA | |
| | TATAATAGTTACAGGAAATCTACCTCTGTGAGAGTGACACCTCTCAGAA | |
| | TGAACTGTGTGACACAAGAAAATGAATGTAGGTCTATCCAAAAAAAAC | |
| | CCCAAGAAACAAAAACAATATTATTAGCCCTTTATGCTTAAGTGATGGA | |
| | CTCAGGGAACAGTTGATGTTGTGATCATTTTATTATCTGATTCTTGTTAC | |
| | TTTGAATTAAACCAATATTTTGATGATATAAATCATTTCCACCAGCATAT | |
| | ATTTAATTTCCATAATAACTTTAAAATTTTCTAATTTCACTCAACTATGA | |
| | GGGAATAGAATGTGGTGGCCACAGGTTTGGCTTTTGTTAAAATGTTTGA | |
| | TATCTTCGATGTTGATCTCTGTCTGCAATGTAGATGTCTAAACACTAGG | |
| | ATTTAATATTTAAGGCTAAGCTTTAAAAATAAAGTACCTTTTTAAAAAG | |
| | AATATGGCTTCACCAAATGGAAAATACCTAATTTCTAAATCTTTTTCTCT | |
| | ACAAAGTCCTATCTACTAATGTCTCCATTACTATTTAGTCATCATAACCA | |
| | TTATCTTCATTTTACATGTCGTGTTCTTTCTGGTAGCTCTAAAATGACAC | |
| | TAAATCATAAGAAGACAGGTTACATATCAGGAAATACTTGAAGGTTAC | |
| | TGAAATAGATTCTTGAGTTAATGAAAATATTTTCTGTAAAAAGGTTTGA | |
| | AAAGCCATTTGAGTCTAAAGCATTATACCTCCATTATCAGTAGTTATGT | |
| | GACAATTGTGTGTGTGTTTAATGTTTAAAGATGTGGCACTTTTTAATAA | |
| | GGCAATGCTATGCTATTTTTTCCCATTTAACATTAAGATAATTTATTGCT | |
| | ATACAGATGATATGGAAATATGATGAACAATATTTTTTTTGCCAAAACT | |
| | ATGCCTTGTAAGTAGCCATGGAATGTCAACCTGTAACTTAAATTATCCA | |
| | CAGATAGTCATGTGTTTGATGATGGGCACTGTGGAGATAACTGACATAG | |
| | GACTGTGCCCCCCTTCTCTGCCACTTACTAGCTGGATGAGATTAAGCAA | |
| | GTCATTTAACTGCTCTGATTAAACCTGCCTTTCCCAAGTGCTTTGTAATG | |
| | AATAGAAATGGAAACCAAAAAAAACGTATACAGGCCTTCAGAAATAGT | |
| | AATTGCTACTATTTTGTTTTCATTAAGCCATAGTTCTGGCTATAATTTTA | |
| | TCAAACTCACCAGCTATATTCTACAGTGAAAGCAGGATTCTAGAAAGTC | |
| | TCACTGTTTTATTTATGTCACCATGTGCTATGATATATTTGGTTGAATTC | |
| | ATTTGAAATTAGGGCTGGAAGTATTCAAGTAATTTCTTCTGCTGAAAAA | |
| | ATACAGTGTTTTGAGTTTAGGGCCTGTTTTATCAAAGTTCTAAAGAGCC | |
| | TATCACTCTTCCATTGTAGACATTTTAAAATAATGACACTGATTTTAACA | |
| | TTTTTAAGTGTCTTTTTAGAACAGAGAGCCTGACTAGAACACAGCCCCT | |
| | CCAAAAACCCATGCTCAAATTATTTTTACTATGGCAGCAATTCCACAAA | |
| | AGGGAACAATGGGTTTAGAAATTACAATGAAGTCATCAACCCAAAAAA | |
| | CATCCCTATCCCTAAGAAGGTTATGATATAAAATGCCCACAAGAAATCT | |
| | ATGTCTGCTTTAATCTGTCTTTTATTGCTTTGGAAGGATGGCTATTACAT | |
| | TTTTAGTTTTTGCTGTGAATACCTGAGCAGTTTCTCTCATCCATACTTAT | |
| | CCTTCACACATCAGAAGTCAGGATAGAATATGAATCATTTTAAAAACTT | |
| | TTACAACTCCAGAGCCATGTGCATAAGAAGCATTCAAAACTTGCCAAA | |
| | ACATACATTTTTTTTCAAATTTAAAGATACTCTATTTTTGTATTCAATAG | |
| | CTCAACAACTGTGGTCCCCACTGATAAAGTGAAGTGGACAAGGAGACA | |
| | AGTAATGGCATAAGTTTGTTTTTCCCAAAGTATGCCTGTTCAATAGCCA | |
| | TTGGATGTGGGAAATTTCTACATCTCTTAAAATTTTACAGAAAATACAT | |
| | AGCCAGATAGTCTAGCAAAAGTTCACCAAGTCCTAAATTGCTTATCCTT | |
| | ACTTCACTAAGTCATGAAATCATTTTAATGAAAAGAACATCACCTAGGT | |
| | TTTGTGGTTTCTTTTTTTCTTATTCATGGCTGAGTGAAAACAACAATCTC | |
| | TGTTTCTCCCTAGCATCTGTGGACTATTTAATGTACCATTATTCCACACT | |
| | CTATGGTCCTTACTAAATACAAAATTGAACAAAAAGCAGTAAAACAAC | |
| | TGACTCTTCACCCATATTATAAAATATAATCCAAGCCAGATTAGTCAAC | |
| | ATCCATAAGATGAATCCAAGCTGAACTGGGCCTAGATTATTGAGTTCAG | |
| | GTTGGATCACATCCCTATTTATTAATAAACTTAGGAAAGAAGGCCTTAC | |
| | AGACCATCAGTTAGCTGGAGCTAATAGAACCTACACTTCTAAAGTTCGG | |
| | CCTAGAATCAATGTGGCCTTAAAAGCTGAAAAGAAGCAGGAAAGAACA | |
| | GTTTTCTTCAATAATTTGTCCACCCTGTCACTGGAGAAAATTTAAGAATT | |
| | TGGGGGTGTTGGTAGTAAGTTAAACACAGCAGCTGTTCATGGCAGAAA | |
| | TTATTCAATACATACCTTCTCTGAATATCCTATAACCAAAGCAAAGAAA | |
| | AACACCAAGGGGTTTGTTCTCCTCCTTGGAGTTGACCTCATTCCAAGGC | |
| | AGAGCTCAGGTCACAGGCACAGGGGCTGCGCCCAAGCTTGTCCGCAGC | |
| | CTTATGCAGCTGTGGAGTCTGGAAGACTGTTGCAGGACTGCTGGCCTAG | |
| | TCCCAGAATGTCAGCCTCATTTTCGATTTACTGGCTCTTGTTGCTGTATG | |
| | TCATGCTGACCTTATTGTTAAACACAGGTTTGTTTGCTTTTTTTCCACTC | |
| | ATGGAGACATGGGAGAGGCATTATTTTTAAGCTGGTTGAAAGCTTTAAC | |
| | CGATAAAGCATTTTTAGAGAAATGTGAATCAGGCAGCTAAGAAAGCAT | |
| | ACTCTGTCCATTACGGTAAAGAAAATGCACAGATTATTAACTCTGCAGT | |
| | GTGGCATTAGTGTCCTGGTCAATATTCGGATAGATATGAATAAAATATT | |
| | TAAATGGTATTGTAAATAGTTTTCAGGACATATGCTATAGCTTATTTTTA | |
| | TTATCTTTTGAAATTGCTCTTAATACATCAAATCCTGATGTATTCAATTT | |
| | ATCAGATATAAATTATTCTAAATGAAGCCCAGTTAAATGTTTTTGTCTTG | |
| | TCAGTTATATGTTAAGTTTCTGATCTCTTTGTCTATGACGTTTACTAATC | |
| | TGCATTTTTACTGTTATGAATTATTTTAGACAGCAGTGGTTTCAAGCTTT | |
| | TTGCCACTAAAAATACCTTTTATTTTCTCCTCCCCCAGAAAAGTCTATAC | |
| | CTTGAAGTATCTATCCACCAAACTGTACTTCTATTAAGAAATAGTTATT | |
| | GTGTTTTCTTAATGTTTTGTTATTCAAAGACATATCAATGAAAGCTGCTG | |
| | AGCAGCATGAATAACAATTATATCCACACAGATTTGATATATTTTGTGC | |
| | AGCCTTAACTTGATAGTATAAAATGTCATTGCTTTTTAAATAATAGTTA | |
| | GTCAATGGACTTCTATCATAGCTTTCCTAAACTAGGTTAAGATCCAGAG | |
| | CTTTGGGGTCATAATATATTACATACAATTAAGTTATCTTTTTCTAAGGG | |
| | CTTTAAAATTCATGAGAATAACCAAAAAAGGTATGTGGAGAGTTAATA | |
| | CAAACATACCATATTCTTGTTGAAACAGAGATGTGGCTCTGCTTGTTCT | |
| | CCATAAGGTAGAAATACTTTCCAGAATTTGCCTAAACTAGTAAGCCCTG | |
| | AATTTGCTATGATTAGGGATAGGAAGAGATTTTCACATGGCAGACTTTA | |
| | GAATTCTTCACTTTAGCCAGTAAAGTATCTCCTTTTGATCTTAGTATTCT | |
| | GTGTATTTTAACTTTTCTGAGTTGTGCATGTTTATAAGAAAAATCAGCAC | |
| | AAAGGGTTTAAGTTAAAGCCTTTTTACTGAAATTTGAAAGAAACAGAA | |
| | GAAAATATCAAAGTTCTTTGTATTTTGAGAGGATTAAATATGATTTACA | |
| | AAAGTTACATGGAGGGCTCTCTAAAACATTAAATTAATTATTTTTTGTT | |
| | GAAAAGTCTTACTTTAGGCATCATTTTATTCCTCAGCAACTAGCTGTGA | |
| | AGCCTTTACTGTGCTGTATGCCAGTCACTCTGCTAGATTGTGGAGATTA | |
| | CCAGTGTTCCCGTCTTCTCCGAGCTTAGAGTTGGATGGGGAATAAAGAC | |
| | AGGTAAACAGATAGCTACAATATTGTACTGTGAATGCTTATGCTGGAGG | |
| | AAGTACAGGGAACTATTGGAGCACCTAAGAGGAGCACCTACCTTGAAT | |
| | TTAGGGGTTAGCAGAGGCATCCTGAAAAAAGTCAAAGCTAAGCCACAA | |
| | TCTATAAGCAGTTTAGGAATTAGCAGAACGTGCGTGGTGAGGAGATGC | |
| | CAAAGGCAAGAAGAGAAGAGTATTCCAAACAGGAGGGATTCCAAAGA | |
| | GAGAAGAGTATCCCAAACAACATTTGCACAAACCTGATGGGGAGAGAG | |
| | AATGTGGGGTGGGGATGGATGATGAGACTGAAGAAGAAAGCCAGGTCT | |
| | AGATAATCAGTGGCCTTGTACACCATGTTAAAGAGTGTAGACTTGATTC | |
| | TGTTGTAAACAGGAAAGCAGCACAATTCATATGAATATTTTAGAAGACT | |
| | CCCACTGGAATATGGAGAATAAAGTTGGAGATGACTAATCCTGGAAGC | |
| | AGGGAGAACATTTTTGAGGAAGTTGCACTATTTTGGTGAAAATGATGAT | |
| | CATAAACATGAAGAATTGTAGGTGATCATGACCTCCTCTCTAATTTTCC | |
| | AGAAGGGTTTTGGAAGATATAACATAGGAACATTGACAGGACTGACGA | |
| | AAGGAGATGAAATACACCATATAAATTGTCAAACACAAGGCCAGATGT | |
| | CTAATTATTTTGCTTATGTGTTGAAATTACAAATTTTTCATCAGGAAACC | |
| | AAAAACTACAAAACTTAGTTTTCCCAAGTCCCAGAATTCTATCTGTCCA | |
| | AACAATCTGTACCACTCCACCTATATCCCTACCTTTGCATGTCTGTCCAA | |
| | CCTCAAAGTCCAGGTCTATACACACGGGTAAGACTAGAGCAGTTCAAG | |
| | TTTCAGAAAATGAGAAAGAGGAACTGAGTTGTGCTGAACCCATACAAA | |
| | ATAAACACATTCTTTGTATAGATTCTTGGAACCTCGAGAGGAATTCACC | |
| | TAACTCATAGGTATTTGATGGTATGAATCCATGGCTGGGCTCGGCTTTT | |
| | AAAAAGCCTTATCTGGGATTCCTTCTATGGAACCAAGTTCCATCAAAGC | |
| | CCATTTAAAAGCCTACATTAAAAACAAAATTCTTGCTGCATTGTATACA | |
| | AATAATGATGTCATGATCAAATAATCAGATGCCATTATCAAGTGGAATT | |
| | ACAAAATGGTATACCCACTCCAAAAAAAAAAAAAAAGCTAAATTCTCA | |
| | GTAGAACATTGTGACTTCATGAGCCCTCCACAGCCTTGGAGCTGAGGAG | |
| | GGAGCACTGGTGAGCAGTAGGTTGAAGAGAAAACTTGGCGCTTAATAA | |
| | TCTATCCATGTTTTTTCATCTAAAAGAGCCTTCTTTTTGGATTACCTTATT | |
| | CAATTTCCATCAAGGAAATTGTTAGTTCCACTAACCAGACAGCAGCTGG | |
| | GAAGGCAGAAGCTTACTGTATGTACATGGTAGCTGTGGGAAGGAGGTT | |
| | TCTTTCTCCAGGTCCTCACTGGCCATACACCAGTCCCTTGTTAGTTATGC | |
| | CTGGTCATAGACCCCCGTTGCTATCATCTCATATTTAAGTCTTTGGCTTG | |
| | TGAATTTATCTATTCTTTCAGCTTCAGCACTGCAGAGTGCTGGGACTTTG | |
| | CTAACTTCCATTTCTTGCTGGCTTAGCACATTCCTCATAGGCCCAGCTCT | |
| | TTTCTCATCTGGCCCTGCTGTGGAGTCACCTTGCCCCTTCAGGAGAGCC | |
| | ATGGCTTACCACTGCCTGCTAAGCCTCCACTCAGCTGCCACCACACTAA | |
| | ATCCAAGCTTCTCTAAGATGTTGCAGACTTTACAGGCAAGCATAAAAGG | |
| | CTTGATCTTCCTGGACTTCCCTTTACTTGTCTGAATCTCACCTCCTTCAA | |
| | CTTTCAGTCTCAGAATGTAGGCATTTGTCCTCTTTGCCCTACATCTTCCT | |
| | TCTTCTGAATCATGAAAGCCTCTCACTTCCTCTTGCTATGTGCTGGAGGC | |
| | TTCTGTCAGGTTTTAGAATGAGTTCTCATCTAGTCCTAGTAGCTTTTGAT | |
| | GCTTAAGTCCACCTTTTAAGGATACCTTTGAGATTTAGACCATGTTTTTC | |
| | GCTTGAGAAAGCCCTAATCTCCAGACTTGCCTTTCTGTGGATTTCAAAG | |
| | ACCAACTGAGGAAGTCAAAAGCTGAATGTTGACTTTCTTTGAACATTTC | |
| | CGCTATAACAATTCCAATTCTCCTCAGAGCAATATGCCTGCCTCCAACT | |
| | GACCAGGAGAAAGGTCCAGTGCCAAAGAGAAAAACACAAAGATTAATT | |
| | ATTTCAGTTGAGCACATACTTTCAAAGTGGTTTGGGTATTCATATGAGG | |
| | TTTTCTGTCAAGAGGGTGAGACTCTTCATCTATCCATGTGTGCCTGACA | |
| | GTTCTCCTGGCACTGGCTGGTAACAGATGCAAAACTGTAAAAATTAAGT | |
| | GATCATGTATTTTAACGATATCATCACATACTTATTTTCTATGTAATGTT | |
| | TTAAATTTCCCCTAACATACTTTGACTGTTTTGCACATGGTAGATATTCA | |
| | CATTTTTTTGTGTTGAAGTTGATGCAATCTTCAAAGTTATCTACCCCGTT | |
| | GCTTATTAGTAAAACTAGTGTTAATACTTGGCAAGAGATGCAGGGAATC | |
| | TTTCTCATGACTCACGCCCTATTTAGTTATTAATGCTACTACCCTATTTT | |
| | GAGTAAGTAGTAGGTCCCTAAGTACATTGTCCAGAGTTATACTTTTAAA | |
| | GATATTTAGCCCCATATACTTCTTGAATCTAAAGTCATACACCTTGCTCC | |
| | TCATTTCTGAGTGGGAAAGACATTTGAGAGTATGTTGACAATTGTTCTG | |
| | AAGGTTTTTGCCAAGAAGGTGAAACTGTCCTTTCATCTGTGTATGCCTG | |
| | GGGCTGGGTCCCTGGCAGTGATGGGGTGACAATGCAAAGCTGTAAAAA | |
| | CTAGGTGCTAGTGGGCACCTAATATCATCATCATATACTTATTTTCAAG | |
| | CTAATATGCAAAATCCCATCTCTGTTTTTAAACTAAGTGTAGATTTCAG | |
| | AGAAAATATTTTGTGGTTCACATAAGAAAACAGTCTACTCAGCTTGACA | |
| | AGTGTTTTATGTTAAATTGGCTGGTGGTTTGAAATGAATCATCTTCACAT | |
| | AATGTTTTCTTTAAAAATATTGTGAATTTAACTCTAATTCTTGTTATTCT | |
| | GTGTGATAATAAAGAATAAACTAATTTCTA | |
| AK093306 | ATTCTATGCTGCAGCCTAAGCATCATTCCTCTTCTCTTCTTAGTGGAGAT | 190 |
| | AAAATTACCCACTGCTCTCCTTACATTTACTTTGTCCATATTTGCTCCTA | |
| | TGCTCTAGGCTCGTGCACAACAAACACAGTGTGGGCCCTTACCCTAGAA | |
| | GCCAACTTCTCATGACCTTTCTCTATCTCCAGAATCCATGCAGTGGGAA | |
| | TGAAGGTAAAAGAAGGTTTTCATGGGATCCAGCTGAGAGCTCTACGGG | |
| | GAAAATGGATCTGAGGAGCCATGTGCTCCATCTCTTTTATTTTACAGGT | |
| | AGAGACTAGGGGTATAGAGTGAGGTGAATTACCGCAGTGACCCACACA | |
| | TTGTTGGCAGACCTAGGATTAGAACTCTGTCTTCCTGGTTCCCAGCTTGG | |
| | TGCTTTTGAAAGCATACTTGCTGCTTTCTTACCGGCCTGGTGTCTGCCAC | |
| | TTTGGGACAGAGTGTGGACTTGCTCACCTGCCCCATTTCTTAGGGATTCT | |
| | CATTCTGTGTTTGAGCAAGAATATTCTTATTCTGGAAAGAACCACATAC | |
| | CACAGGATTCTGGGTGAGCATAAGGAAGATTGTCTTGGGGATCTGACTT | |
| | AGCTCACGTATAGTGGCTATGATGAATTCAGTGTCTTATTTTTTGCATAT | |
| | GTATATTTTTAGTCTAATATTGCCTGGGTGTCTGAGCAAGTCTAGATGA | |
| | ATTTAATTGCTCTCATTTTTCCCCTGCCCCTCTTCCTTTGGTCTCTCTTTT | |
| | AGGAAATGTTTTTCTTTCAACATTCGTTTCATTCATTATTTACTCATTCG | |
| | GCCAACCAACATTTATTGAGTGCCTTCCCTGTATCAGGGACAGGGGCTT | |
| | ACAAAGTAGAATTTGATCCCACCTCTGCCCTCAGTAGCTCAGTGTCTAA | |
| | TGGAGGTAGTGATGTTCATTAAGCGTCGCCAGATACTGTGCTAGGTGCT | |
| | GTGCCTGTTCTCTCTCGCTTGTTCCTCACACACTTGAGAAGGCCGAAGCT | |
| | GATTCATAGCTTGGAAGGCAGGGGCCTTGGATTTGAACCCAGGCCTGAC | |
| | CAATGGCAGAACCTATCAGATGTGTGGACAGATGACATTGCCTTTCTTT | |
| | CTTTGGATATATCAAAATCAGCCAGCAGGCAGGAACTCCCATTTTGAGC | |
| | AAGCAATGTGCAGGAATGATAGGGTATACAGAGAGGAACAGGAGATG | |
| | GCCCCTGACTTCCAGCATGTGTCTGATGGACATCCAGGCTGCAGGCATC | |
| | ATGGTGCTGTCTAGAGAGATGAGCCAGGTGCCCAGAGCCCATGGGCCA | |
| | ATGCTGCCCTTTCTTGAGCATGCCAAACAAAGCGGTTGGTGTGTTAGAG | |
| | GCACAGTCTCCTCCACTCTAAGTAAAAATCAGCATGAGTCCTAGCCCAC | |
| | ATTTCCCTAGTGAGTACACCAAAGATATCTATGAACTGGCAGTCATCAG | |
| | TGACTTCCTAAGGTTCCGGAAATGCATCTCTTACTCAGGAGTAAGCAAT | |
| | GATGTGCCTGCGGCTTTACGAGTTCTCACAGAATGACTTTCTGGACCCA | |
| | AATGTTTTTTCTGCTTCAGGACTGTGAAGGCCTTATTGTTCGCTCTGCCA | |
| | CCAAGGTGACCGCTGATGTCATCAACGCAGCTGAGAAACTCCAGGTGG | |
| | TGGGCAGGGCTGGCACAGGTGTGGACAATGTGGATCTGGAGGCCGCAA | |
| | CAAGGAAGGGCATCTTGGTTATGAACACCCCCAATGGGAACAGCCTCA | |
| | GTGCCGCAGAACTCACTTGTGGAATGATCATGTGCCTGGCCAGGCAGAT | |
| | TCCCCAGGCGACGGCTTCGATGAAGGACGGCAAATGGGAGCGGAAGAA | |
| | GTTCATGGGAACAGAGCTGAATGGAAAGACCCTGGGAATTCTTGGCCT | |
| | GGGCAGGATTGGGAGAGAGGTAGCTACCCGGATGCAGTCCTTTGGGAT | |
| | GAAGACTATAGGGTATGACCCCATCATTTCCCCAGAGGTCTCGGCCTCC | |
| | TTTGGTGTTCAGCAGCTGCCCCTGGAGGAGATCTGGCCTCTCTGTGATTT | |
| | CATCACTGTGCACACTCCTCTCCTGCCCTCCACGACAGGCTTGCTGAAT | |
| | GACAACACCTTTGCCCAGTGCAAGAAGGGGGTGCGTGTGGTGAACTGT | |
| | GCCCGTGGAGGGATCGTGGACGAAGGCGCCCTGCTCCGGGCCCTGCAG | |
| | TCTGGCCAGTGTGCCGGGGCTGCACTGGACGTGTTTACGGAAGAGCCGC | |
| | CACGGGACCGGGCCTTGGTGGACCATGAGAATGTCATCAGCTGTCCCCA | |
| | CCTGGGTGCCAGCACCAAGGAGGCTCAGAGCCGCTGTGGGGAGGAAAT | |
| | TGCTGTTCAGTTCGTGGACATGGTGAAGGGGAAATCTCTCACGGGGGTT | |
| | GTGAATGCCCAGGCCCTTACCAGTGCCTTCTCTCCACACACCAAGCCTT | |
| | GGATTGGTCTGGCAGAAGCTCTGGGGACACTGATGCGAGCCTGGGCTG | |
| | GGTCCCCCAAAGGGACCATCCAGGTGATAACACAGGGAACATCCCTGA | |
| | AGAATGCTGGGAACTGCCTAAGCCCCGCAGTCATTGTCGGCCTCCTGAA | |
| | AGAGGCTTCCAAGCAGGCGGATGTGAACTTGGTGAACGCTAAGCTGCT | |
| | GGTGAAAGAGGCTGGCCTCAATGTCACCACCTCCCACAGCCCTGCTGCA | |
| | CCAGGGGGGCAAGGCTTCGGGGAATGCCTCCTGGCCGTGGCCCTGGCA | |
| | GGCGCCCCTTACCAGGCTGTGGGCTTGGTCCAAGGCACTACACCTGTAC | |
| | TGCAGGGGCTCAATGGAGCTGTCTTCAGGCCAGAAGTGCCTCTCCGCAG | |
| | GGACCTGCCCCTGCTCCTATTCCGGACTCAGACCTCTGACCCTGCAATG | |
| | CTGCCTACCATGATTGGCCTCCTGGCAGAGGCAGGCGTGCGGCTGCTGT | |
| | CCTACCAGACTTCACTGGTGTCAGATGGGGAGACCTGGCACGTCATGGG | |
| | CATCTCCTCCTTGCTGCCCAGCCTGGAAGCGTGGAAGCAGCATGTGACT | |
| | GAAGCCTTCCAGTTCCACTTCTAACCTTGGAGCTCACTGGTCCCTGCCTC | |
| | TGGGGCTTTTCTGAAGAAACCCACCCACTGTGATCAATAGGGAGAGAA | |
| | AATCCACATTCTTGGGCTGAACGCGAGCCTCTGACACTGCTTACACTGC | |
| | ACTCTGACCCTGTAGTACAGCAATAACCGTCTAATAAAGAGCCTACCCC | |
| | C | |
| BE904476 | CAAACAAAAACAGCCAAGCTTTTCTGCCAAAAAGATGACTGAGAAGAC | 191 |
| | TGTTAAAGCAAAAAGCTCTGTTCCTGCCTCAGATGATGCCTATCCAGAA | |
| | ATAGAAAAATTCTTTCCCTTCAATCCTCTAGACTTTGAGAGTTTTGACCT | |
| | GCCTGAAGAGCACCAGATTGCGCACCTCCCCTTGAGTGGAGTGCCTCTC | |
| | ATGATCCTTGACGAGGAGAGAGAGCTTGAAAAGCTGTTTCAGCTGGGC | |
| | CCCCCTTCACCTGTGAAGATGCCCTCTCCACCATGGGAATCCAATCTGT | |
| | TGCAGTCTCCTTCAAGCATTCTGTCGACCCTGGATGTTGAATTGCCACCT | |
| | GTTTGCTGTGACATAGATATTTAAATTTCTTAGTGCTTCAGAGTCTGTGT | |
| | GTATTTGTATTAATAAAGCATTCTTTAACAGAAAAAAAAAAAAAAAAA | |
| | AAAAAAAAAAAAAAAAAAAAAAAAAAAAAAGGGGGGGGAGACACAA | |
| | AAAGAATTCCCCAAGAGGGGGCCACAAGATAATCAGAGGATATCACAC | |
| | AAGATCTCTCGGCGCACCAACGACGGGGGCCCCAAATAAGGGAGAGAC | |
| | CCAGAATCACAACAGCCAAGACACGGTGGACACGACGGAAACAAACA | |
| | CACAGCCCAGACACGGGGGCAAACACGCGCGCACACCGCGGACACCAT | |
| | GGGACAAAGCAGACACCACCCACAAAACAACACCGCGGAGGGGGAAG | |
| | AACAACAAAACAAGTGCGCAAACAGAACACAACCACAGAAAGAGAAA | |
| | AATTAAAACGGCCCCCAAGACGGCGACAACACAACAAAACAACCACTA | |
| | CAGAGCGCTCAACAGCCGAGTAAAAACACAACAACGGACAACTAACAC | |
| | ACAAAGGAATGAAACAAAGCGGGGCCACACACCGACACCGGAAATCC | |
| | GGCGAACAACTCACACCGAGCGAGGGTCCCAGACAACAAATACACAGA | |
| | CAACGAAACCGAGAAACAAGACCAGCAAGACGAGCAGGCAAAAGACA | |
| | AACAAGACAGAGGAGACGACGACGAACGCAAAGGACAAGAGGACACA | |
| | ACGACGCGAGGAGCGAGAGCGAGAGGAAGAGACAACAAAAAGACACA | |
| | AAAGAACAACAAGCAAGCAGCGAAGAACGACACACAACCACACGAGA | |
| | CAGCAGGAGCAGAGGCGGAGAAAACACAACGAGCAAGCCAAGACCAA | |
| | GAGAGGAGAACAAAATAAAAAAATACGAGAGCAGGCGGACGAGAGCA | |
| | CGAGACGAACAGACAAACGGGAATCAGAAGCATAACGATCCGCGACG | |
| | CGAACAACN | |
| AK123010 | GTGCACCCTGTCCCAGCCGTCCTGTCCTGGCTGCTCGCTCTGCTTCGCTG | 192 |
| | CGCCTCCACTATGCTCTCCCTCCGTGTCCCGCTCGCGCCCATCACGGACC | |
| | CGCAGCAGCTGCAGCTCTCGCCGCTGAAGGGGCTCAGCTTGGTCGACA | |
| | AGGAGAACACGCCGCCGGCCCTGAGCGGGACCCGCGTCCTGGCCAGCA | |
| | AGACCGCGAGGAGGATCTTCCAGGAGAAAACCCCCGCCGCTTTGTCAT | |
| | CTTCCCCATCGAGTACCATGATATCTGGCAGATGTATAAGAAGGCAGAG | |
| | GCTTCCTTTTGGACCGCCGAGGAGGTGGACCTCTCCAAGGACATTCAGC | |
| | ACTGGGAATCCCTGAAACCCGAGGAGAGATATTTTATATCCCATGTTCT | |
| | GGCTTTCTTTGCAGCAAGCGATGGCATAGTAAATGAAAACTTGGTGGAG | |
| | CGATTTAGCCAAGAAGTTCAGATTACAGAAGCCCGCTGTTTCTATGGCT | |
| | TCCAAATTGCCATGGAAAACATACATTCTGAAATGTATAGTCTTCTTAT | |
| | TGACACTTACATAAAAGATCCCAAAGAAAGGGAATTTCTCTTCAATGCC | |
| | ATTGAAACGATGCCTTGTGTCAAGAAGAAGGCAGACTGGGCCTTGCGC | |
| | TGGATTGGGGACAAAGAGGCTACCTATGGTGAACGTGTTGTAGCCTTTG | |
| | CTGCAGTGGAAGGCATTTTCTTTTCCGGTTCTTTTGCGTCGATATTCTGG | |
| | CTCAAGAAACGAGGACTGATGCCTGGCCTCACATTTTCTAATGAACTTA | |
| | TTAGCAGAGATGAGGGTTTACACTGTGATTTTGCTTGCCTGATGTTCAA | |
| | ACACCTGGTACACAAACCATCGGAGGAGAGAGTAAGAGAAATAATTAT | |
| | CAATGCTGTTCGGATAGAACAGGAGTTCCTCACTGAGGCCTTGCCTGTG | |
| | AAGCTCATTGGGATGAATTGCACTCTAATGAAGCAATACATTGAGTTTG | |
| | TGGCAGACAGACTTATGCTGGAACTGGGTTTTAGCAAGGTTTTCAGAGT | |
| | AGAGAACCCATTTGACTTTATGGAGAATATTTCACTGGAAGGAAAGACT | |
| | AACTTCTTTGAGAAGAGAGTAGGCGAGTATCAGAGGATGGGAGTGATG | |
| | TCAAGTCCAACAGAGAATTCTTTTACCTTGGATGCTGACTTCTAAATGA | |
| | ACTGAAGATGTGCCCTTACTTGGCTGATTTTTTTTTTTCCATCTCATAAG | |
| | AAAAATCAGCTGAAGTGTTACCAACTAGCCACACCATGAATTGTCCGTA | |
| | ATGTTCATTAACAGCATCTTTAAAACTGTGTAGCTACCTCACAACCAGT | |
| | CCTGTCTGTTTATAGTGCTGGTAGTATCACCTTTTGCCAGAAGGCCTGGC | |
| | TGGCTGTGACTTACCATAGCAGTGACAATGGCAGTCTTGGCTTTAAAGT | |
| | GAGGGGTGACCCTTTAGTGAGCTTAGCACAGCGGGATTAAACAGTCCTT | |
| | TAACCAGCACAGCCAGTTAAAAGATGCAGCCTCACTGCTTCAACGCAG | |
| | ATTTTAATGTTTACTTAAATATAAACCTGGCACTTTACAAACAAATAAA | |
| | CATTGTTTGTACTCACAAGGCGATAATAGCTTGATTTATTTGGTTTCTAC | |
| | ACCAAATACATTCTCCTGACCACTAATGGGAGCCAATTCACAATTCACT | |
| | AAGTGACTAAAGTAAGTTAAACTTGTGTAGACTAAGCATGTAATTTTTA | |
| | AGTTTTATTTTAATGAATTAAAATATTTGTTAACCAACTTTAAAGTCAGT | |
| | CCTGTGTATACCTAGATATTAGTCAGTTGGTGCCAGATAGAAGACAGGT | |
| | TGTGTTTTTATCCTGTGGCTTGTGTAGTGTCCTGGGATTCTCTGCCCCCT | |
| | CTGAGTAGAGTGTTGTGGGATAAAGGAATCTCTCAGGGCAAGGAGCTT | |
| | CTTAAGTTAAATCACTAGAAATTTAGGGGTGATCTGGGCCTTCATATGT | |
| | GTGAGAAGCCGTTTCATTTTATTTCTCACTGTATTTTCCTCAACGTCTGG | |
| | TTGATGAGAAAAAATTCTTGAAGAGTTTTCATATGTGGGAGCTAAGGTA | |
| | GTATTGTAAAATTTCAAGTCATCCTTAAACAAAATGATCCACCTAAGAT | |
| | CTTGCCCCTGTTAAGTGGTGAAATCAACTAGAGGTGGTTCCTACAAGTT | |
| | GTTCATTCTAGTTTTGTTTGGTGTAAGTAGGTTGTGTGAGTTAATTCATT | |
| | TATATTTACTATGTCTGTTAAATCAGAAATTTTTTATTATCTATGTTCTTC | |
| | TAGATTTTACCTGTAGTTCATACTTCAGTCACCCAGTGTCTTATTCTGGC | |
| | ATTGTCTAAATCTGAGCATTGTCTAGGGGGATCTTAAACTTTAGTAGGA | |
| | AACCATGAGCTGTTAATACAGTTTCCATTCAAATATTAATTTCAGAATG | |
| | AAACATAATTTTTTTTTTTTTTTTTTGAGATGGAGTCTCGCTCTGTTGCCC | |
| | AGGCTGGAGTGCAGTGGCGCGATTTTGGCTCACTGTAACCTCCATCTCC | |
| | TGGGTTCAAGCAATTCTCCTGTCTCAGCCTCCCTAGTAGCTGGGACTGC | |
| | AGGTATGTGCTACCACACCTGGCTAATTTTTGTATTTTTAGTAGAGATG | |
| | GAGTTTCACCATATTGGTCAGGCTGGTCTTGAACTCCTGACCTCAGGTG | |
| | ATCCACCCACCTCGGCCTCCCAAAGTGCTGGGATTGCAGGCGTGATAAA | |
| | CAAATATTCTTAATAGGGCTACTTTGAATTAATCTGCCTTTATGTTTGGG | |
| | AGAAGAAAGCTGAGACATTGCATGAAAGATGATGAGAGATAAATGTTG | |
| | ATCTTTTGGCCCCATTTGTTAATTGTATTCAGTATTTGAACGTCGTCCTG | |
| | TTTATTGTTAGTTTTCTTCATCATTTATTGTATAGACAATTTTTAAATCTC | |
| | TGTAATATGATACATTTTCCTATCTTTTAAGTTATTGTTACCTAAAGTTA | |
| | ATCCAGATTATATGGTCCTTATATGTGTACAACATTAAAATGAAAGGCT | |
| | TTGTCTTGCATTGTGAGGTACAGGCGGAAGTTGGAATCAGGTTTTAGGA | |
| | TTCTGTCTCTCATTAGCTGAATAATGTGAGGATTAACTTCTGCCAGCTCA | |
| | GACCATTTCCTAATCAGTTGAAAGGGAAACAAGTATTTCAGTCTCAAAA | |
| | TTGAATAATGCACAAGTCTTAAGTGATTAAAATAAAACTGTTCTTATGT | |
| | CAGTTT | |
| BC036503 | AGCGGGGGCACTCCAGCCCTGCAGCCTCCGGAGTCAGTGCCGCGCGCC | 193 |
| | CGCCGCCCCGCGCCTTCCTGCTCGCCGCACCTCCGGGAGCCGGGGCGCA | |
| | CCCAGCCCGCAGCGCCGCCTCCCCGCCCGCGCCGCCTCCGACCGCAGGC | |
| | CGAGGGCCGCCACTGGCCGGGGGGACCGGGCAGCAGCTTGCGGCCGCG | |
| | GAGCCGGGCAACGCTGGGGACTGCGCCTTTTGTCCCCGGAGGTCCCTGG | |
| | AAGTTTGCGGCAGGACGCGCGCGGGGAGGCGGCGGAGGCAGCCCCGAC | |
| | GTCGCGGAGAACAGGGCGCAGAGCCGGCATGGGCATCGGGCGCAGCG | |
| | AGGGGGGCCGCCGCGGGGCAGCCCTGGGCGTGCTGCTGGCGCTGGGCG | |
| | CGGCGCTTCTGGCCGTGGGCTCGGCCAGCGAGTACGACTACGTGAGCTT | |
| | CCAGTCGGACATCGGCCCGTACCAGAGCGGGCGCTTCTACACCAAGCC | |
| | ACCTCAGTGCGTGGACATCCCCGCGGACCTGCGGCTGTGCCACAACGTG | |
| | GGCTACAAGAAGATGGTGCTGCCCAACCTGCTGGAGCACGAGACCATG | |
| | GCGGAGGTGAAGCAGCAGGCCAGCAGCTGGGTGCCCCTGCTCAACAAG | |
| | AACTGCCACGCCGGCACCCAGGTCTTCCTCTGCTCGCTCTTCGCGCCCG | |
| | TCTGCCTGGACCGGCCCATCTACCCGTGTCGCTGGCTCTGCGAGGCCGT | |
| | GCGCGACTCGTGCGAGCCGGTCATGCAGTTCTTCGGCTTCTACTGGCCC | |
| | GAGATGCTTAAGTGTGACAAGTTCCCCGAGGGGGACGTCTGCATCGCC | |
| | ATGACGCCGCCCAATGCCACCGAAGCCTCCAAGCCCCAAGGCACAACG | |
| | GTGTGTCCTCCCTGTGACAACGAGTTGAAATCTGAGGCCATCATTGAAC | |
| | ATCTCTGTGCCAGCGAGTTTGCACTGAGGATGAAAATAAAAGAAGTGA | |
| | AAAAAGAAAATGGCGACAAGAAGATTGTCCCCAAGAAGAAGAAGCCC | |
| | CTGAAGTTGGGGCCCATCAAGAAGAAGGACCTGAAGAAGCTTGTGCTG | |
| | TACCTGAAGAATGGGGCTGACTGTCCCTGCCACCAGCTGGACAACCTCA | |
| | GCCACCACTTCCTCATCATGGGCCGCAAGGTGAAGAGCCAGTACTTGCT | |
| | GACGGCCATCCACAAGTGGGACAAGAAAAACAAGGAGTTCAAAAACTT | |
| | CATGAAGAAAATGAAAAACCATGAGTGCCCCACCTTTCAGTCCGTGTTT | |
| | AAGTGATTCTCCCGGGGGCAGGGTGGGGAGGGAGCCTCGGGTGGGGTG | |
| | GGAGCGGGGGGGACAGTGCCCCGGGAACCCGGTGGGTCACACACACGC | |
| | ACTGCGCCTGTCAGTAGTGGACATTTAATCCAGTCGGCTTGTTCTTGCA | |
| | GCATTCCCGCTCCCTTCCCTCCATAGCCACGCTCCAAACCCCAGGGTAG | |
| | CCATGGCCGGGTAAAGCAAGGGCCATTTAGATTAGGAAGGTTTTTAAG | |
| | ATCCGCAATGTGGAGCAGCAGCCACTGCACAGGAGGAGGTGACAAACC | |
| | ATTTCCAACAGCAACACAGCCACTAAAACACAAAAAGGGGGATTGGGC | |
| | GGAAAGTGAGAGCCAGCAGCAAAAACTACATTTTGCAACTTGTTGGTG | |
| | TGGATCTATTGGCTGATCTATGCCTTTCAACTAGAAAATTCTAATGATTG | |
| | GCAAGTCACGTTGTTTTCAGGTCCAGAGTAGTTTCTTTCTGTCTGCTTTA | |
| | AATGGAAACAGACTCATACCACACTTACAATTAAGGTCAAGCCCAGAA | |
| | AGTGATAAGTGCAGGGAGGAAAAGTGCAAGTCCATTATGTAATAGTGA | |
| | CAGCAAAGGGACCAGGGGAGAGGCATTGCCTTCTCTGCCCACAGTCTTT | |
| | CCGTGTGATTGTCTTTGAATCTGAATCAGCCAGTCTCAGATGCCCCAAA | |
| | GTTTCGGTTCCTATGAGCCCGGGGCATGATCTGATCCCCAAGACATGTG | |
| | GAGGGGCAGCCTGTGCCTGCCTTTGTGTCAGAAAAAGGAAACCACAGT | |
| | GAGCCTGAGAGAGACGGCGATTTTCGGGCTGAGAAGGCAGTAGTTTTC | |
| | AAAACACATAGTTAAAAAAGAAACAAATGAAAAAAATTTTAGAACAGT | |
| | CCAGCAAATTGCTAGTCAGGGTGAATTGTGAAATTGGGTGAAGAGCTT | |
| | ACGATTCTAATCTCATGTTTTTTCCTTTTCACATTTTTAAAAGAACAATG | |
| | ACAAACACCCACTTATTTTTCAAGGTTTTAAAACAGTCTACATTGAGCA | |
| | TTTGAAAGGTGTGCTAGAACAAGGTCTCCTGATCCGTCCGAGGCTGCTT | |
| | CCCAGAGGAGCAGCTCTCCCCAGGCATTTGCCAAGGGAGGCGGATTTC | |
| | CCTGGTAGTGTAGCTGTGTGGCTTTCCTTCCTGAAGAGTCCGTGGTTGCC | |
| | CTAGAACCTAACACCCCCTAGCAAAACTCACAGAGCTTTCCGTTTTTTT | |
| | CTTTCCTGTAAAGAAACATTTCCTTTGAACTTGATTGCCTATGGATCAAA | |
| | GAAATTCAGAACAGCCTGCCTGTCCCCCCGCACTTTTTACATATATTTGT | |
| | TTCATTTCTGCAGATGGAAAGTTGACATGGGTGGGGTGTCCCCATCCAG | |
| | CGAGAGAGTTTAAAAAGCAAAACATCTCTGCAGTTTTTCCCAAGTGCCC | |
| | TGAGATACTTCCCAAAGCCCTTATGTTTAATCAGCGATGTATATAAGCC | |
| | AGTTCACTTAGACAACTTTACCCTTCTTGTCCAATGTACAGGAAGTAGT | |
| | TCTAAAAAAAATGCATATTAATTTCTTCCCCCAAAGCCGGATTCTTAAT | |
| | TCTCTGCAACACTTTGAGGACATTTATGATTGTCCCTCTGGGCCAATGCT | |
| | TATACCCAGTGAGGATGCTGCAGTGAGGCTGTAAAGTGGCCCCCTGCG | |
| | GCCCTAGCCTGACCCGGAGGAAAGGATGGTAGATTCTGTTAACTCTTGA | |
| | AGACTCCAGTATGAAAATCAGCATGCCCGCCTAGTTACCTACCGGAGA | |
| | GTTATCCTGATAAATTAACCTCTCACAGTTAGTGATCCTGTCCTTTTAAC | |
| | ACCTTTTTTGTGGGGTTCTCTCTGACCTTTCATCGTAAAGTGCTGGGGAC | |
| | CTTAAGTGATTTGCCTGTAATTTTGGATGATTAAAAAATGTGTATATAT | |
| | ATTAGCTAATTAGAAATATTCTACTTCTCTGTTGTCAAACTGAAATTCAG | |
| | AGCAAGTTCCTGAGTGCGTGGATCTGGGTCTTAGTTCTGGTTGATTCAC | |
| | TCAAGAGTTCAGTGCTCATACGTATCTGCTCATTTTGACAAAGTGCCTC | |
| | ATGCAACCGGGCCCTCTCTCTGCGGCAGAGTCCTTAGTGGAGGGGTTTA | |
| | CCTGGAACATTAGTAGTTACCACAGAATACGGAAGAGCAGGTGACTGT | |
| | GCTGTGCAGCTCTCTAAATGGGAATTCTCAGGTAGGAAGCAACAGCTTC | |
| | AGAAAGAGCTCAAAATAAATTGGAAATGTGAATCGCAGCTGTGGGTTT | |
| | TACCACCGTCTGTCTCAGAGTCCCAGGACCTTGAGTGTCATTAGTTACTT | |
| | TATTGAAGGTTTTAGACCCATAGCAGCTTTGTCTCTGTCACATCAGCAA | |
| | TTTCAGAACCAAAAGGGAGGCTCTCTGTAGGCACAGAGCTGCACTATC | |
| | ACGAGCCTTTGTTTTTCTCCACAAAGTATCTAACAAAACCAATGTGCAG | |
| | ACTGATTGGCCTGGTCATTGGTCTCCGAGAGAGGAGGTTTGCCTGTGAT | |
| | TTCCTAATTATCGCTAGGGCCAAGGTGGGATTTGTAAAGCTTTACAATA | |
| | ATCATTCTGGATAGAGTCCTGGGAGGTCCTTGGCAGAACTCAGTTAAAT | |
| | CTTTGAAGAATATTTGTAGTTATCTTAGAAGATAGCATGGGAGGTGAGG | |
| | ATTCCAAAAACATTTTATTTTTAAAATATCCTGTGTAACACTTGGCTCTT | |
| | GGTACCTGTGGGTTAGCATCAAGTTCTCCCCAGGGTAGAATTCAATCAG | |
| | AGCTCCAGTTTGCATTTGGATGTGTAAATTACAGTAATCCCATTTCCCA | |
| | AACCTAAAATCTGTTTTTCTCATCAGACTCTGAGTAACTGGTTGCTGTGT | |
| | CATAACTTCATAGATGCAGGAGGCTCAGGTGATCTGTTTGAGCAGAGCA | |
| | CCCTAGGCAGCCTGCAGGGAATAACATACTGGCCGTTCTGACCTGTTGC | |
| | CAGCAGATACACAGGACATGGATGAAATTCCCGTTTCCTCTAGTTTCTT | |
| | CCTGTAGTACTCCTCTTTTAGATCCTAAGTCTCTTACAAAAGCTTTGAAT | |
| | ACTGTGAAAATGTTTTACATTCCATTTCATTTGTGTTGTTTTTTTAACTGC | |
| | ATTTTACCAGATGTTTTGATGTTATCGCTTATGTTAATAGTAATTCCCGT | |
| | ACGTGTTCATTTTATTTTCATGCTTTTTCAGCCATGTATCAATATTCACTT | |
| | GACTAAAATCACTCAATTAATCAAAAAAAAAAAAAAAA | |
| NM_012319 | AGTCCTGGGCGAAGGGGGCGGTGGTTCCCCGCGGCGCTGCGCGCGGCG | 194 |
| | GTAATTAGTGATTGTCTTCCAGCTTCGCGAAGGCTAGGGGCGCGGCTGC | |
| | CGGGTGGCTGCGCGGCGCTGCCCCCGGACCGAGGGGCAGCCAACCCAA | |
| | TGAAACCACCGCGTGTTCGCGCCTGGTAGAGATTTCTCGAAGACACCAG | |
| | TGGGCCCGTTCCGAGCCCTCTGGACCGCCCGTGTGGAACCAAACCTGCG | |
| | CGCGTGGCCGGGCCGTGGGACAACGAGGCCGCGGAGACGAAGGCGCA | |
| | ATGGCGAGGAAGTTATCTGTAATCTTGATCCTGACCTTTGCCCTCTCTGT | |
| | CACAAATCCCCTTCATGAACTAAAAGCAGCTGCTTTCCCCCAGACCACT | |
| | GAGAAAATTAGTCCGAATTGGGAATCTGGCATTAATGTTGACTTGGCAA | |
| | TTTCCACACGGCAATATCATCTACAACAGCTTTTCTACCGCTATGGAGA | |
| | AAATAATTCTTTGTCAGTTGAAGGGTTCAGAAAATTACTTCAAAATATA | |
| | GGCATAGATAAGATTAAAAGAATCCATATACACCATGACCACGACCAT | |
| | CACTCAGACCACGAGCATCACTCAGACCATGAGCGTCACTCAGACCAT | |
| | GAGCATCACTCAGACCACGAGCATCACTCTGACCATGATCATCACTCTC | |
| | ACCATAATCATGCTGCTTCTGGTAAAAATAAGCGAAAAGCTCTTTGCCC | |
| | AGACCATGACTCAGATAGTTCAGGTAAAGATCCTAGAAACAGCCAGGG | |
| | GAAAGGAGCTCACCGACCAGAACATGCCAGTGGTAGAAGGAATGTCAA | |
| | GGACAGTGTTAGTGCTAGTGAAGTGACCTCAACTGTGTACAACACTGTC | |
| | TCTGAAGGAACTCACTTTCTAGAGACAATAGAGACTCCAAGACCTGGA | |
| | AAACTCTTCCCCAAAGATGTAAGCAGCTCCACTCCACCCAGTGTCACAT | |
| | CAAAGAGCCGGGTGAGCCGGCTGGCTGGTAGGAAAACAAATGAATCTG | |
| | TGAGTGAGCCCCGAAAAGGCTTTATGTATTCCAGAAACACAAATGAAA | |
| | ATCCTCAGGAGTGTTTCAATGCATCAAAGCTACTGACATCTCATGGCAT | |
| | GGGCATCCAGGTTCCGCTGAATGCAACAGAGTTCAACTATCTCTGTCCA | |
| | GCCATCATCAACCAAATTGATGCTAGATCTTGTCTGATTCATACAAGTG | |
| | AAAAGAAGGCTGAAATCCCTCCAAAGACCTATTCATTACAAATAGCCT | |
| | GGGTTGGTGGTTTTATAGCCATTTCCATCATCAGTTTCCTGTCTCTGCTG | |
| | GGGGTTATCTTAGTGCCTCTCATGAATCGGGTGTTTTTCAAATTTCTCCT | |
| | GAGTTTCCTTGTGGCACTGGCCGTTGGGACTTTGAGTGGTGATGCTTTTT | |
| | TACACCTTCTTCCACATTCTCATGCAAGTCACCACCATAGTCATAGCCAT | |
| | GAAGAACCAGCAATGGAAATGAAAAGAGGACCACTTTTCAGTCATCTG | |
| | TCTTCTCAAAACATAGAAGAAAGTGCCTATTTTGATTCCACGTGGAAGG | |
| | GTCTAACAGCTCTAGGAGGCCTGTATTTCATGTTTCTTGTTGAACATGTC | |
| | CTCACATTGATCAAACAATTTAAAGATAAGAAGAAAAAGAATCAGAAG | |
| | AAACCTGAAAATGATGATGATGTGGAGATTAAGAAGCAGTTGTCCAAG | |
| | TATGAATCTCAACTTTCAACAAATGAGGAGAAAGTAGATACAGATGAT | |
| | CGAACTGAAGGCTATTTACGAGCAGACTCACAAGAGCCCTCCCACTTTG | |
| | ATTCTCAGCAGCCTGCAGTCTTGGAAGAAGAAGAGGTCATGATAGCTC | |
| | ATGCTCATCCACAGGAAGTCTACAATGAATATGTACCCAGAGGGTGCA | |
| | AGAATAAATGCCATTCACATTTCCACGATACACTCGGCCAGTCAGACGA | |
| | TCTCATTCACCACCATCATGACTACCATCATATTCTCCATCATCACCACC | |
| | ACCAAAACCACCATCCTCACAGTCACAGCCAGCGCTACTCTCGGGAGG | |
| | AGCTGAAAGATGCCGGCGTCGCCACTCTGGCCTGGATGGTGATAATGG | |
| | GTGATGGCCTGCACAATTTCAGCGATGGCCTAGCAATTGGTGCTGCTTT | |
| | TACTGAAGGCTTATCAAGTGGTTTAAGTACTTCTGTTGCTGTGTTCTGTC | |
| | ATGAGTTGCCTCATGAATTAGGTGACTTTGCTGTTCTACTAAAGGCTGG | |
| | CATGACCGTTAAGCAGGCTGTCCTTTATAATGCATTGTCAGCCATGCTG | |
| | GCGTATCTTGGAATGGCAACAGGAATTTTCATTGGTCATTATGCTGAAA | |
| | ATGTTTCTATGTGGATATTTGCACTTACTGCTGGCTTATTCATGTATGTT | |
| | GCTCTGGTTGATATGGTACCTGAAATGCTGCACAATGATGCTAGTGACC | |
| | ATGGATGTAGCCGCTGGGGGTATTTCTTTTTACAGAATGCTGGGATGCT | |
| | TTTGGGTTTTGGAATTATGTTACTTATTTCCATATTTGAACATAAAATCG | |
| | TGTTTCGTATAAATTTCTAGTTAAGGTTTAAATGCTAGAGTAGCTTAAA | |
| | AAGTTGTCATAGTTTCAGTAGGTCATAGGGAGATGAGTTTGTATGCTGT | |
| | ACTATGCAGCGTTTAAAGTTAGTGGGTTTTGTGATTTTTGTATTGAATAT | |
| | TGCTGTCTGTTACAAAGTCAGTTAAAGGTACGTTTTAATATTTAAGTTAT | |
| | TCTATCTTGGAGATAAAATCTGTATGTGCAATTCACCGGTATTACCAGT | |
| | TTATTATGTAAACAAGAGATTTGGCATGACATGTTCTGTATGTTTCAGG | |
| | GAAAAATGTCTTTAATGCTTTTTCAAGAACTAACACAGTTATTCCTATA | |
| | CTGGATTTTAGGTCTCTGAAGAACTGCTGGTGTTTAGGAATAAGAATGT | |
| | GCATGAAGCCTAAAATACCAAGAAAGCTTATACTGAATTTAAGCAAAG | |
| | AAATAAAGGAGAAAAGAGAAGAATCTGAGAATTGGGGAGGCATAGAT | |
| | TCTTATAAAAATCACAAAATTTGTTGTAAATTAGAGGGGAGAAATTTAG | |
| | AATTAAGTATAAAAAGGCAGAATTAGTATAGAGTACATTCATTAAACA | |
| | TTTTTGTCAGGATTATTTCCCGTAAAAACGTAGTGAGCACTTTTCATATA | |
| | CTAATTTAGTTGTACATTTAACTTTGTATAATACAGAAATCTAAATATAT | |
| | TTAATGAATTCAAGCAATATATCACTTGACCAAGAAATTGGAATTTCAA | |
| | AATGTTCGTGCGGGTATATACCAGATGAGTACAGTGAGTAGTTTTATGT | |
| | ATCACCAGACTGGGTTATTGCCAAGTTATATATCACCAAAAGCTGTATG | |
| | ACTGGATGTTCTGGTTACCTGGTTTACAAAATTATCAGAGTAGTAAAAC | |
| | TTTGATATATATGAGGATATTAAAACTACACTAAGTATCATTTGATTCG | |
| | ATTCAGAAAGTACTTTGATATCTCTCAGTGCTTCAGTGCTATCATTGTGA | |
| | GCAATTGTCTTTTATATACGGTACTGTAGCCATACTAGGCCTGTCTGTGG | |
| | CATTCTCTAGATGTTTCTTTTTTACACAATAAATTCCTTATATCAGCTTG | |
| | AAAAAAAAAAAAAAAAAA | |
| AK098106 | AACGCACTTGGCGCGCGGCGCGGGCTGCAGACGGCTGCGAGGCGCTGG | 195 |
| | GCACAGGTGTCCTGATGGCAAATTTCAAGGGCCACGCGCTTCCAGGGA | |
| | GTTTCTTCCTGATCATTGGGCTGTGTTGGTCAGTGAAGTACCCGCTGAA | |
| | GTACTTTAGCCACACGCGGAAGAACAGCCCACTACATTACTATCAGCGT | |
| | CTCGAGATCGTCGAAGCCGCAATTAGGACTTTGTTTTCCGTCACTGGGA | |
| | TCCTGGCAGAGCAGTTTGTTCCGGATGGGCCCCACCTGCACCTCTACCA | |
| | TGAGAACCACTGGATAAAGTTAATGAATTGGCAGCACAGCACCATGTA | |
| | CCTATTCTTTGCAGTCTCAGGAATTGTTGACATGCTCACCTATCTGGTCA | |
| | GCCACGTTCCCTTGGGGGTGGACAGACTGGTTATGGCTGTGGCAGTATT | |
| | CATGGAAGGTTTCCTCTTCTACTACCACGTCCACAACCGGCCTCCGCTG | |
| | GACCAGCACATCCACTCACTCCTGCTGTATGCTCTGTTCGGAGGGTGTG | |
| | TTAGTATCTCCCTAGAGGTGATCTTCCGGGACCACATTGTGCTGGAACT | |
| | TTTCCGAACCAGTCTCATCATTCTTCAGGGAACCTGGTTCTGGCAGATT | |
| | GGGTTTGTGCTGTTCCCACCTTTTGGAACACCCGAATGGGACCAGAAGG | |
| | ATGATGCCAACCTCATGTTCATCACCATGTGCTTCTGCTGGCACTACCTG | |
| | GCTGCCCTCAGCATTGTGGCCGTCAACTATTCTCTTGTTTACTGCCTTTT | |
| | GACTCGGATGAAGAGACACGGAAGGGGAGAAATCATTGGAATTCAGAA | |
| | GCTGAATTCAGATGACACTTACCAGACCGCCCTCTTGAGTGGCTCAGAT | |
| | GAGGAATGAGCCGAGATGCGGAGGGCGCAGATGTCCCACTGCACAGCT | |
| | GGAATGAATGGAGTTCATCCCCTCCACCTGAATGCCTGCTGTGGTCTGA | |
| | TCTTAAGGGTCTATATATTTGCACCTCCTCATTCAACACAGGGCTGGAG | |
| | GTTCTACAACAGGAAATCAGGCCTACAGCATCCTGTGTATCTTGCAGTT | |
| | GGGATTTTTAAACATACTATAAAGTCTGTGTTGGTATAGTACCCTTCAT | |
| | AAGGAAAAATGAAGTAATGCCTATAAGTAGCAGGCCTTTGTGCCTCAG | |
| | TGTCAAGAGAAATCAAGAGATGCTAAAAGCTTTACAATGGAAGTGGCC | |
| | TCATGGATGAATCCGGGGTATGAGCCCAGGAGAACGTGCTGCTTTTGGT | |
| | AACTTATCCCTTTTTCTCTTAAGAAAGCAGGTACTTTCTTATTAGAAATA | |
| | TGTTAGAATGTGTAAGCAAACGACAGTGCCTTTAGAATTACAATTCTAA | |
| | CTTACATATTTTTTGAAAGTAAAATAATTCACAAGCTTTGGTATTTTAAA | |
| | ATTATTGTTAAACATATCATAACTAATCATACCAGGGTACTGCAATACC | |
| | ACTGTTTATAAGTGACAAAATTAGGCCAAAGGTGATTTTTTTTTAAATC | |
| | AGGAAGCTGGTTACTGGCTCTACTGAGAGTTGGAGCCCTGATGTTCTGA | |
| | TTCTTCAAAGTCACCCTAAAAGAAGATCTGACAGGAAAGCTGTATAATG | |
| | AGATAGAAAAACGTCAGGTATGGAAGGCTTTCAGTTTTAATATGGCTGA | |
| | AAGCAAAGGATAACGAATTCAGAATTAGTAATGTAAAATCTTGATACC | |
| | CTAATCTTGCTTCTGGATCTGTTCTTTTTTTAAAAAAACTTCCTTCACCG | |
| | CGCCTATAATCCTAGCACTTTGGGAGGCCGAGGCAGGCAGATCACGGG | |
| | GTCAGGAGATCAAGACCATCCTGGCTAACATGGTGAAACCCCGTCTCTA | |
| | CTGAAAATACAAAAAATTAGCCGGGTGTGGTGGCGGGCGCCTGTAGTT | |
| | CCAGCTACTCGGGAGGCTGAGGCAAGAGAATGGCATGAACCCGGTAGG | |
| | GGAGCTTGCAGTGAGCCCAGATCATGCCACTGTACTCCAGCCTAGGTGA | |
| | CAGAGCAAGACTCTGTCTCAAAAACAAGCAAACAGACTTCCTTCAACA | |
| | AATATTTATTAAATATCCACTTTGCAACAGCACTGAAATGGCTGTAAGG | |
| | ACTCCTGAGATATGTGTCCAGCAAGGAGTTTACAGTCAAACAGGAGAG | |
| | ACATGCCTGTAGTTACATCCAGTGTGATGGGTGCTGAGAGGCAAGTACA | |
| | AACCACGATG | |
| BQ056428 | TCCCGCCGCGCCACTTCGCCTGCCTCCGTCCCCCGCCCGCCGCGCCATG | 196 |
| | CCTGTGGCCGGCTCGGAGCTGCCGCGCCGGCCCTTGCCCCCCGCCGCAC | |
| | AGGAGCGGGACGCCGAGCCGCGTCCGCCGCACGGGGAGCTGCAGTACC | |
| | TGGGGCAGATCCAACACATCCTCCGCTGCGGCGTCAGGAAGGACGCCC | |
| | GCCCGGGCACCGGTACCCTGCCGGTATTCGGCATGCAGGCGCGCTACA | |
| | GCCTGAGAGATGAATTCCCTCTGCTGACAACCAAACGTGTGTTCTGGAA | |
| | CGGTGCTTCGGAGGAGCTGCTGTGGCTTATCAAGGGATCCACAAACGCT | |
| | ATAGACCTGTCTTCCCCGGCAGCGAAAATCTCGGGATGCCACTGGATCC | |
| | CGACACTCTCTGGACACCCTGGGATTCTCCACCAGAGAAGAACGCGACT | |
| | TGGGCCCAGTTTGTGGCTCTCAGCGGAGGCCTCCTGTGGCAGAATACAT | |
| | ACATTTCCAATCAGATCACTTCCCGGACACGGACCNTGACCAGCCTGCC | |
| | AAAAAGTGGATTTCCCCCCACCCCAGAACCCANCCCCTGACGCACAGA | |
| | AACCAACCCATTCGTTGTTGCCGCCTTGCGAACCCCAACCAGAATCTCT | |
| | CCCCCCTGGCCGGCGCGCCTGCCGCTGCCAATGCCCCTATGGCGGCCTC | |
| | TTGGCCCGCACCTTCCAATTGGTCGCCCTGCGCAACCAGCGAGAAAACA | |
| | CTGGCCCGCCCGTCTCCCCCCCGCTCCGCCTACCCCACTTAATGCGCCTC | |
| | CGTGGCATGACGCACGCGTTTGGTGTCCGCCGCCGTCTCATGTCCGCGC | |
| | GGTGTGGACCCCCTTTTCTCTCGCGGCACATCCCCCCTATTCCCTTGCCC | |
| | TTTGGGGGGCACCCCCTCTAGACCCGCGCTTCTCTTCTCGTCCGGTGGG | |
| | GGACATTGGTTTGCCTGCCGCGGCGGGGGCGNTAAAAATAAAAACAGC | |
| | CTGTTAGCCCGGCCCAGTACCCCCCCCCGGCCGGGGCCGCCTTNCGTTT | |
| | GCATTTATACCCCAACCCATAAAGCCGCGCCCCTTTAGCNCCNTAACTT | |
| | TTGTGGTGTGGCCTCCCCCCTTTTTCCCGGGGAGCAGCAACGGACATCT | |
| | GTACACTAATGCTGGCCCCGACCTTTCCCAAAAACCCCCCGCCCGTGTC | |
| | CCGTATAAATTTGGTGCCAANCCTGACGNGTTCTCCCCCGCCCTCGCCC | |
| | CGTTGGCCGCCCGTTTAAAGCCCCCCCGGTGGTTGCGCCGCCCAACGAG | |
| | TCCACCTATAGTTAANTCCACCAACACCCCCACCTTTTCCTCCCCGCCGC | |
| | ATCTTCCCCACGTACCCCCTTTTGTCGCGAGATGGCCACTCCCCCCCCCC | |
| | TGTTTGTTTAAAACAACGAGAATGGTGCTGCCAACGCTGGTCTTTTCCC | |
| | CCCCCGGACCGCGACCGCCAGGGGGAATACGTACCATAAGCCCCCGCG | |
| | CCCNCCTTTTTTCCCCCCTCCCCGCCAATCAAGATCCGCCGTCCATTAGA | |
| | CGTATTATTTTTCCCGCGATACACGAAAAAACAGGGCCGCCCATTTATA | |
| | ACTAAATTCCCGTCGCCGCCGCGCGGATATGTTTCCCAAAATACCACCC | |
| | CCCCCCCCCCATTTTCTTTGCCCCCAACTCCTGCGCACCGGTGTTCACCA | |
| | GCCTCGCGCCGC | |
| BC032677 | GGACGCGTGGGTCGACCCACGCGTCCGGACCCACGCGTCCGGTCGTGTT | 197 |
| | CTCCGAGTTCCTGTCTCTCTGCCAACGCCGCCCGGATGGCTTCCCAAAA | |
| | CCGCGACCCAGCCGCCACTAGCGTCGCCGCCGCCCGTAAAGGAGCTGA | |
| | GCCGAGCGGGGGCGCCGCCCGGGGTCCGGTGGGCAAAAGGCTACAGCA | |
| | GGAGCTGATGACCCTCATGGTGAGTGATTAAGTGCCCAGAACCCCAGC | |
| | CTTCCATCCAATTTTCAGTAGCCTCCTTTTTTCCGTCAGCTTTTTTGCTAG | |
| | ACATAGGGGTAATGTAATTTGCTCCCTCCTGGGAAAGAAGTTCATACAC | |
| | CCCACCTACACCATTTCTTCCAGCAGTCCCTCCTCCCAATTCCATCCCCC | |
| | CACACGAAGTTATCTCGAACACTTCCCTGAAGTCATACAAGACCCTCCC | |
| | TATCCAGTGTGTCCCTACTTCCTAGCCCCAACCAAGCTTTACCCACACCC | |
| | AACTCCCCGCCCTTCTTGGTATTTCTAGCCTATGAATTTGGTTGCTTTAT | |
| | TTTGGATCAGAGTGATGAGATTAAGGGGAGGCTGGGCGCGGTAGCTCA | |
| | CACCTTATAATCCCAAAGTGCTGGGATTACAGGCGTGAGCCACCGCGCC | |
| | CGGCCAGCAACTAATATTCTAATTGAACTAAAGCACAGGATGCCAATTT | |
| | ACAATCCTTAGACCAAAGAGTCACTGATGTCTCCACCAGATAAGAGGA | |
| | AAGCATCAGGCTAGGCATAGTGGCTCACACCTGTAATCTCAGCACTTTG | |
| | GGAGGCTGAGGCAGGCAGATCACATGAGCCCAGGAGTTTGAGACTGGC | |
| | CTGGGCAACATGGTGAAACCCTGTCTCTAAAATAAAAACTAAACTAAA | |
| | AAAACTTTTTAAAAAGGCAGTGGGGAGCATCAGAACCAGCTCAACAGT | |
| | TTGTCTACTGTCCGGTCCCAGAGAAACTCAAGATTCTAGCAAGCCCCTT | |
| | GTGTGGGGCTTGGGTTGGGACATGAGGCTGCTGCTGGAGCTTACTCTGC | |
| | AACTGTTTCTCCAAATGCCAGGTATATGAAGACCTGAGGTATAAGCTCT | |
| | CGCTAGAGTTCCCCAGTGGCTACCCTTACAATGCGCCCACAGTGAAGTT | |
| | CCTCACGCCCTGCTATCACCCCAACGTGGACACCCAGGGTAACATATGC | |
| | CTGGACATCCTGAAGGAAAAGTGGTCTGCCCTGTATGATGTCAGGACCA | |
| | TTCTGCTCTCCATCCAGAGCCTTCTAGGAGAACCCAACATTGATAGTCC | |
| | CTTGAACACACATGCTGCCGAGCTCTGGAAAAACCCCACAGCTTTTAAG | |
| | AAGTACCTGCAAGAAACCTACTCAAAGCAGGTCACCAGCCAGGAGCCC | |
| | TGACCCAGGCTGCCCAGCCTGTCCTTGTGTCGTCTTTTTAATTTTTCCTT | |
| | AGATGGTCTGTCCTTTTTGTGATTTCTGTATAGGACTCTTTATCTTGAGC | |
| | TGTGGTATTTTTGTTTTGTTTTTGTCTTTTAAATTAAGCCTCGGTTGAGCC | |
| | CTTGTATATTAAATAAATGCATTTTTGTCCTTTTTTAAAAAAAAAATAAA | |
| | AAAAAAAAAAAAAAAAAAAAAAAAAAAAAAAAAAAAAAAAAA | |

The NANO46 gene expression assay, as described herein, is able to identify intrinsic subtype from standard formalin fixed paraffin embedded tumor tissue (also see, Parker et al. J. Clin Oncol., 27(8):1160-7 (2009) and U.S. Patent Application Publication No. 2013/0337444 The methods utilize a supervised algorithm to classify subject samples according to breast cancer intrinsic subtype. This algorithm, referred to herein as the “NANO46 classification model”, is based on the gene expression profile of a defined subset of intrinsic genes that has been identified herein as superior for classifying breast cancer intrinsic subtypes; see, U.S. Patent Application Publication No. 2013/0337444. In particular, expression of 46 of the genes listed in Table 1 is determined (which is by determining the expression of all 50 genes in Table 1 with the exception of determining the expression of MYBL2, BIRC5, GRB7 and CCNB1), i.e., the “NANO46” set of genes. The skilled artisan can utilize any primer and/or target sequence-specific probe for detecting any of (or each of) the genes in Table 1.

At least 10, at least 15, at least 20, at least 25, at least 40, at least 41, at least 42, at least 43, at least 44, at least 46, at least 47, at least 48, at least 49 or all 50 of the genes in Table 1 can be utilized in the methods and kits of the present invention. Preferably, the expression of each of the 50 genes is determined in a biological sample. More preferably, the expression of each of the genes in the NANO46 set of genes is determined in a biological sample. The prototypical gene expression profiles (i.e., centroid) of the four intrinsic subtypes were pre-defined from a training set of formalin fixed paraffin embedded tissues (FFPE) breast tumor samples using hierarchical clustering analysis of gene expression data. Table 4 shows the actual values of the prototypical gene expression profiles (i.e., centroids) of these four subtypes and for a normal sample.

TABLE 4
Table 4. Subtype Centroids for Comparison to a Sample
Subtype Centroids for Comparison to a Sample

| | | Her2- | | | |
|---|---|---|---|---|---|
| Target Gene | Basal-like | enriched | Luminal A | Luminal B | Normal |
| ACTR3B | −0.2052 | −0.7965 | −0.2790 | −0.4380 | 0.6676 |
| ANLN | 1.0227 | 0.5006 | −0.7289 | 0.1149 | −1.7879 |
| BAG1 | −0.4676 | −0.3132 | 0.4716 | 0.5879 | −0.3280 |
| BCL2 | −0.7365 | −0.7237 | 0.7234 | 0.6363 | 0.5144 |
| BIRC5 | 0.9542 | 0.4541 | −0.6921 | 0.3421 | −1.6821 |
| BLVRA | −0.8761 | 0.2270 | 0.1628 | 0.7138 | −0.2665 |
| CCNB1 | 0.7337 | 0.3114 | −0.8626 | 0.2165 | −1.5967 |
| CCNE1 | 1.3100 | 0.2201 | −0.6231 | −0.2729 | −1.0925 |
| CDC20 | 1.0995 | 0.1445 | −1.0518 | −0.1173 | −1.2069 |
| CDC6 | 0.5817 | 0.6601 | −0.7032 | 0.3134 | −1.2255 |
| CDCA1 | 0.9367 | 0.1623 | −0.4509 | 0.2692 | −1.9055 |
| CDH3 | 0.7639 | 0.0144 | −0.0502 | −1.0229 | 0.5007 |
| CENPF | 1.0222 | 0.2944 | −0.5657 | 0.2437 | −1.8612 |
| CEP55 | 1.0442 | 0.4881 | −0.6365 | 0.2921 | −1.9241 |
| CXXC5 | −0.9732 | 0.1866 | 0.5687 | 0.9463 | −0.3030 |
| EGFR | 0.3352 | −0.1326 | −0.0011 | −0.9755 | 1.4238 |
| ERBB2 | −0.7045 | 1.4182 | 0.2420 | 0.1978 | −0.5530 |
| ESR1 | −1.1847 | −0.4926 | 0.7177 | 1.0101 | 0.0087 |
| EXO1 | 1.0546 | 0.4317 | −0.7259 | 0.2559 | −1.6488 |
| FGFR4 | −0.2073 | 1.4562 | 0.1707 | −0.2223 | −0.5802 |
| FOXA1 | −1.3590 | 0.5726 | 0.7131 | 0.7963 | −0.2353 |
| FOXC1 | 1.0666 | −0.7362 | −0.4078 | −0.9877 | 0.6650 |
| GPR160 | −1.0540 | 0.5524 | 0.6032 | 0.7305 | −0.3224 |
| GRB7 | −0.4848 | 1.3418 | 0.0124 | 0.0690 | −0.2520 |
| KIF2C | 0.9242 | 0.1104 | −1.1001 | −0.2771 | −1.3455 |
| KNTC2 | 1.1373 | 0.2266 | −0.7593 | 0.1656 | −1.1881 |
| KRT14 | 0.4759 | −0.5269 | 0.8187 | −0.8879 | 1.1352 |
| KRT17 | 0.6863 | −0.3777 | 0.6149 | −1.1415 | 0.9238 |
| KRT5 | 0.7136 | −0.4146 | 0.5832 | −0.9462 | 1.0985 |
| MAPT | −1.1343 | −0.2711 | 1.0957 | 0.8372 | 0.4007 |
| MDM2 | −0.7498 | −0.4855 | −0.1788 | 0.2397 | 0.1097 |
| MELK | 1.0209 | 0.2678 | −0.8016 | 0.1012 | −1.6272 |
| MIA | 1.2408 | −0.5475 | 0.3289 | −0.6320 | 0.6975 |
| MKI67 | 1.0446 | 0.4630 | −0.6717 | 0.3161 | −1.7680 |
| MLPH | −1.4150 | 0.4842 | 0.8829 | 0.8194 | −0.2419 |
| MMP11 | −0.1295 | 0.5220 | 0.3402 | 0.5653 | −1.7370 |
| MYBL2 | 0.9571 | 0.5492 | −0.7814 | 0.1548 | −1.4404 |
| MYC | 0.5639 | −0.9904 | −0.3015 | −0.2791 | 0.9833 |
| NAT1 | −0.9711 | −0.2708 | 1.2256 | 0.9576 | −0.5287 |
| ORC6L | 1.0086 | 0.5152 | −1.0385 | −0.0336 | −1.4084 |
| PGR | −0.9216 | −0.5755 | 1.2061 | 0.9278 | 0.6220 |
| PHGDH | 0.9192 | 0.0322 | −0.5194 | −0.5371 | 0.5184 |
| PTTG1 | 0.9541 | 0.2079 | −1.1207 | 0.1052 | −1.4067 |
| RRM2 | 0.7895 | 0.6336 | −0.8099 | 0.3228 | −1.7630 |
| SFRP1 | 0.7694 | −0.8271 | 0.2617 | −1.0846 | 1.3790 |
| SLC39A6 | −0.9992 | −0.4573 | 0.6607 | 0.9222 | −0.2463 |
| TMEM45B | −1.0721 | 0.7926 | 0.3190 | 0.2016 | −0.2250 |
| TYMS | 0.9823 | −0.0960 | −0.8593 | 0.1827 | −1.3192 |
| UBE2C | 0.8294 | 0.3358 | −1.0141 | 0.0608 | −1.7637 |
| UBE2T | 0.6258 | 0.0617 | −0.8652 | −0.0487 | −1.8602 |

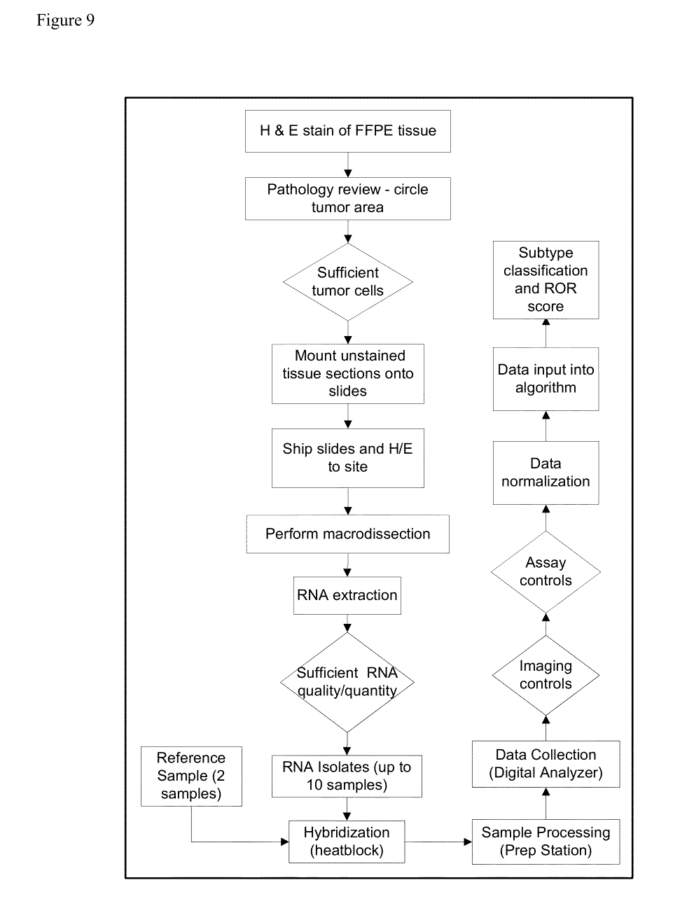

FIG. 9 outlines the assay processes associated with the Breast Cancer Intrinsic Subtyping test. Following RNA isolation, the test will simultaneously measure the expression levels of at least 40 target genes (e.g., 46 or 50) plus eight housekeeping genes. For example, the housekeeping genes described in U.S. Patent Publication 2008/0032293 can be used for normalization. Exemplary housekeeping genes include MRPL19, PSMC4, SF3A1, PUM1, ACTB, GAPD, GUSB, RPLP0, and TFRC. The housekeeping genes are used to normalize the expression of the tumor sample. Each assay run may also include a reference sample consisting of in vitro transcribed RNA's of the target genes and the housekeeping genes for normalization purposes.

After performing the Breast Cancer Intrinsic Subtyping test with a test breast cancer tumor sample and the reference sample provided as part of a test kit or as used in a method, a computational algorithm based on a Pearson's correlation compares the normalized and scaled gene expression profile of the at least 40 genes or the PAM50 or NANO46 intrinsic gene sets of the test sample to the prototypical expression signatures of the four breast cancer intrinsic subtypes. See, U.S. Patent Application Publication Nos. 2011/0145176 and 2013/0337444. In embodiments, the intrinsic subtype analysis is determined by determining the expression of a PAM50 or NANO46 sets of genes and the risk of recurrence (“ROR”) is determined using the NANO46 set of genes (which is determining the expression of all 50 genes in Table 1 with the exception of determining the expression of MYBL2, BIRC5, GRB7 and CCNB1). Specifically, the intrinsic subtype is identified by comparing the expression of the at least 40 genes or the PAM50 or NANO46 set of genes in the biological sample with the expected expression profiles for the four intrinsic subtypes. The subtype with the most similar expression profile is assigned to the biological sample. The ROR score is an integer value on a 0-100 scale that is related to an individual patient's probability of distant recurrence within 10 years for the defined intended use population. The ROR score is calculated by comparing the expression profiles of the at least 40 genes, e.g., the NANO46 genes, in the biological sample with the expected profiles for the four intrinsic subtypes, as described above, to calculate four different correlation values. These correlation values may then be combined with a proliferation score (and optionally one or more clinicopathological variables, such as tumor size) to calculate the ROR score. Preferably, the ROR score is calculated by comparing only the expression profiles of the NANO46 genes.

A ROR score can be calculated using any method or formula known in the art. Exemplary formulae include Equations 1 to 6, as described herein.

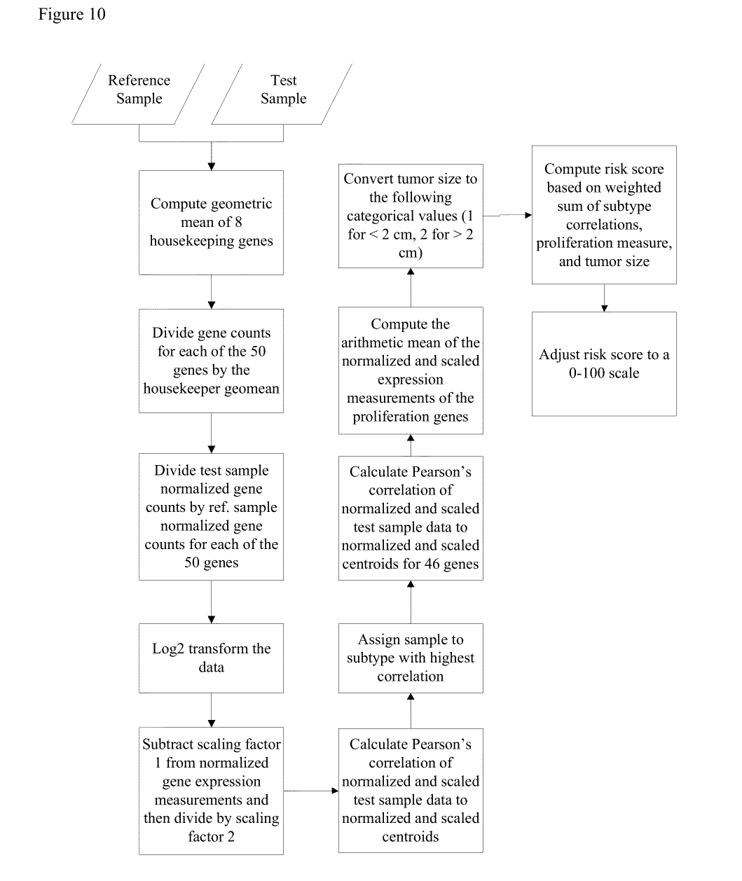

FIG. 10 provides a schematic of specific algorithm transformations. The tumor sample is assigned the subtype with the largest positive correlation to the sample. Kaplan Meier survival curves are generated from a training set of untreated breast cancer patients demonstrate that the intrinsic subtypes are a prognostic indicator of recurrence free survival (RFS).

The training set of formalin fixed paraffin embedded tissues (FFPE) breast tumor samples, which had well defined clinical characteristics and clinical outcome data, were used to establish a continuous Risk of Recurrence (ROR) score. The score is calculated using coefficients from a Cox model that includes correlation to each intrinsic subtype, a proliferation score (mean gene expression of a subset of 18 of the 46 genes), and tumor size. See, Table 5.

TABLE 5
Coefficients to calculate ROR-PT (Equation 1)

| Test Variables | Coefficient |
|---|---|
| Basal-like Pearson's correlation (A) | −0.0067 |
| HER2-enriched Pearson's correlation (B) | 0.4317 |
| Luminal A Pearson's correlation (C) | −0.3172 |
| Luminal B Pearson's correlation (D) | 0.4894 |
| Proliferation Score (E) | 0.1981 |
| Tumor Size (F) | 0.1133 |

The test variables in Table 5 are multiplied by the corresponding coefficients and summed to produce a risk score (“ROR-PT”) as shown in the following equation (Equation 1):

ROR-PT equation=−0.0067*A+0.4317*B+−0.3172*C+0.4894*D+0.1981*E+0.1133*F.

In previous studies, the ROR score provided a continuous estimate of the risk of recurrence for ER-positive, node-negative patients who were treated with tamoxifen for 5 years (Nielsen et al. Clin. Cancer Res., 16(21):5222-5232 (2009)). The ROR score also exhibited a statistically significant improvement over a clinical model based in determining relapse-free survival (RFS) within this test population providing further evidence of the improved accuracy of this decision making tool when compared to traditional clinicopathological measures (Nielsen et al. Clin. Cancer Res., 16(21):5222-5232 (2009)).

The ROR score is an integer value on a 0-100 scale that is related to an individual patient's probability of distant recurrence within 10 years for the defined intended use population. The ROR score is calculated by comparing the expression profiles of 46 genes in an unknown sample with the expected profiles for the four intrinsic subtypes, as described above, to calculate four different correlation values. These correlation values are then combined with a proliferation score and the tumor size to calculate the ROR score. Risk classification is also provided to allow interpretation of the ROR score by using cutoffs related to clinical outcome in tested patient populations. See, Table 6.

TABLE 6
Risk classification by ROR range and nodal status

| Nodal Status | ROR Range | Risk Classification |
|---|---|---|
| Node-Negative | 0-40 | Low |
| | 41-60 | Intermediate |
| | 61-100 | High |
| Node-Positive | 0-15 | Low |
| (1-3 nodes) | 16-40 | Intermediate |
| | 41-100 | High |

The methods and kits of the present invention can further include steps and/or reagents for providing a VEGF-signature score. The VEGF-signature score can be determined from the expression of at least one of, a combination of, or each of, a 13-gene set of genes associated with VEGF signaling or regulation. The 13-gene set includes RRAGD, FABP5, UCHL1, GAL, PLOD, DDIT4, VEGF, ADM, ANGPTL4, NDRG1, NP, SLC16A3, and C14ORF 58. Table 7 provides the Genbank Accession Numbers and select nucleic acid sequences of the 13-gene set for determining the VEGF-signature score.

TABLE 7
VEGF Signature Score Gene Set

| GENE NAME | | |
|---|---|---|
| GENBANK | | SEQ |
| ACCESSION | | ID |
| NUMBER | SEQUENCE | NO: |
| RRAGD | CGCAGTCTCTCTCTCTCTCCCTCCTCCGGGAGGAACTGCCGCGCTCCGGCT | 198 |
| NM_021244.4 | GACTCCTCCGCCGGCGGGCGGGGCGGGGGAGGGGGCTTCGGGCGCGCTG | |
| | GGAACCGCGGGACCCGGACCTGGGCGCCGCCCGCCGGGGGACGCGCGGC | |
| | CCCCGCTTCCGCCGGGCCCCGCTGAGCTCTAGACAAACCTCCGCTTCAGA | |
| | AATAGGCTGCGGGCGGCCGGCTAGGAGGCTTGGCCCCCACCCCGGGACC | |
| | CCCGCCGTCCCCGGGCCGGCCGGCCGGTGGGCACGATGAGCCAGGTGCTG | |
| | GGGAAGCCGCAGCCGCAGGACGAGGACGACGCGGAGGAGGAGGAGGAG | |
| | GAGGATGAGCTGGTGGGGCTAGCGGACTACGGAGACGGGCCCGACTCCT | |
| | CCGACGCCGATCCGGACAGCGGCACAGAGGAGGGAGTTCTGGACTTCAG | |
| | TGACCCCTTCAGCACTGAAGTGAAGCCGAGAATCCTGCTCATGGGCCTGA | |
| | GGAGAAGCGGCAAGTCGTCTATTCAGAAAGTTGTCTTTCACAAAATGTCT | |
| | CCCAACGAAACTCTGTTCTTGGAGAGCACTAATAAGATATGCCGGGAAGA | |
| | TGTTTCCAACAGCTCCTTTGTCAATTTTCAGATTTGGGACTTCCCAGGACA | |
| | GATTGACTTTTTTGACCCTACATTTGACTATGAGATGATCTTCCGGGGAAC | |
| | AGGAGCACTGATATTTGTCATTGACTCACAGGATGATTACATGGAAGCCC | |
| | TGGCCAGGCTCCACCTCACGGTGACCAGGGCCTACAAAGTGAATACTGAC | |
| | ATCAACTTCGAGGTGTTTATTCATAAAGTGGATGGTCTGTCAGATGACCA | |
| | CAAAATTGAAACCCAAAGAGATATTCACCAGAGGGCAAACGATGACCTT | |
| | GCAGATGCTGGATTAGAAAAAATTCACCTCAGCTTTTATCTGACAAGCAT | |
| | ATATGATCATTCAATATTTGAAGCTTTTAGCAAAGTTGTTCAGAAACTGAT | |
| | TCCACAACTCCCAACTCTGGAGAATTTGCTGAACATCTTTATCTCAAATTC | |
| | TGGAATTGAAAAGGCATTTCTATTTGATGTGGTCAGTAAAATTTATATTGC | |
| | AACTGATAGTACTCCGGTGGATATGCAAACCTATGAGCTCTGCTGTGATA | |
| | TGATAGATGTGGTTATTGACATCTCTTGTATTTATGGTCTCAAAGAAGATG | |
| | GAGCAGGAACCCCCTATGACAAGGAATCCACAGCCATCATAAAGCTTAAT | |
| | AATACAACCGTGCTTTATTTAAAAGAGGTGACAAAGTTCCTGGCTCTCGT | |
| | TTGCTTTGTCAGAGAGGAAAGCTTTGAAAGAAAAGGGCTAATTGACTATA | |
| | ATTTTCATTGCTTCCGGAAGGCCATTCATGAAGTTTTTGAGGTGAGAATGA | |
| | AAGTAGTAAAATCTCGAAAGGTTCAGAATCGGCTGCAGAAGAAAAAGAG | |
| | AGCCACCCCTAATGGGACCCCTAGAGTGCTGCTGTAGGTGAGGTTTCAGG | |
| | AATGTCTTTTGAAATCAGACCTTATCCATGAGGCTGCTGCGCCATGTTGCA | |
| | CTAAAGGAAGAGGAAGAAGGAGATTGGGACACATACCATTGATTTGTTG | |
| | TTAAAAAAAAAAAATTCCTGCAACCCTCTTGATCTTCTCTTTTATAAATAA | |
| | AGTAAGCACTTTGAAGCAAAAACTTGTATATTAACAGTGAATGTGAAATCC | |
| | ATTGTCATTTCATTACACAAATGTAAACTTTTATGGTCTGTAGTCAAAAAA | |
| | ATCCCGTGTGAGAACTGCCAGGAATTGTACATATTTTGCACTTTTTCATGT | |
| | TTCTCATTGAACTGAACTTTGATAAAACGACTTTTCTAAGCTTTTTTTCTGT | |
| | ACTTGGTGTCAAGGACATGCATACTGTAGTCCATATCTATATGGCAATCA | |
| | GAAATTAATCAAAAAGTGATGCATTGGTAATGACTTTTTGTAAATTTGGA | |
| | AATCTTTGCTACCAATTGTTGAGAAAAATCATTTTTCAGTGGAGCTGGAA | |
| | CAGATTGGAGCTACAAGCTCCAGGAGCAATAAGAACTGTCCCCTATTTAT | |
| | AATGGGTGTAAACAGTTTTGTAGAATAATGCTAGCACCAGACTTACCTAA | |
| | AAATTTCTATAGCAGTGGCTGTGCTTCCCTGCTCAACGGTTTTTATGAAGC | |
| | TGTTTACCTCAACACACATCTCTATAATCACTTTATACAGAGAGGTTATTT | |
| | CTTTTTGTTGCATTAGTATTCTTTTGAAACTTTGGGACCAGATTTCCAAAA | |
| | TGGTGCCGAACACTGGAGAGAAGTAAGAATGTCACTGAATTGTAGGGTTT | |
| | CTGGAGGCTTTTCTGTACCTACCACCCAGGGCTAAAGTAACATCAGAGGC | |
| | CTAAAGTTGTTCCAAAAGTATGTGATTGGCAACTGCAGACTAAAAAACAT | |
| | AGATACAATTCTGGACTTTTGGCCCTGTGCGATGGTCTGGTGTGCTGCATT | |
| | TAAAATGCTTATTCAGGACCAGTTCTTTATTGCTCCATGACCATAGTGAAT | |
| | AGAACAAATCGCAGAACCCCACCATGGAGCTCAATCCTGTTAGTCACTTT | |
| | TGTCACCTCCACATCTCCTTCTCACTGGTGATAACATGCCTCATGTACTCC | |
| | ACTTGTTCCCACCTTATGATTAAGCCAAGCTCAGCCTGCCACCAGCATGCT | |
| | CTGCAAGGCTGAAGAGTCATCCTGAAGACCCCTAAAGGTCAGTGGGAAA | |
| | AGGATGGCTGGAGAGACATTACAATTAGCTGTGTAATTGTTTCTGTGAAA | |
| | TTATTTCACTTATGTTTACTTTAGACTAACAGGAAATTAAGAGTCCTAAAT | |
| | CTACCCCTATGCCAAATCATTCCAAGTAGATAATTTTACGTGCATCTCAAG | |
| | GGTTAGCACCCTAAGGCATGCTTGTGGGGCATTAGAAAATGAGATTTTTT | |
| | TTTTTTTAAAGCAGAGCCTCCTAAGAACATCAAAGTTGGTCCTAGCAAAA | |
| | TATATAAAGTCCCTAAAGCAACTTATACTTGAAACTTTTTTTTTTTTTTTTT | |
| | TTAGAGGGGGGCCTCATTCTGTTGCTTGTGCTGGAATACATTGGTACAATC | |
| | ATAGCTCACTGTTACCTCTTGGGCTCAAGGGATCCTTCCACCTCAGCCTCC | |
| | CTAGTAGCTAGAACTACAGGTGTGCACAACCACGCCCGGCTAATTCTTAAZ | |
| | ATTTTGTTTTTGTAGAGACAGGATCTCACTGTGTTGCCCAAGATGGTCTCA | |
| | AACTCCTGGCCCCAAGCAATCCTCCTGCCTTGGCCTCCTCAAATGCTGAG | |
| | ATTACAGGCCTGAGCTACTGTGCCCAGCCTAAACTTTCCCACTTCTCTCTG | |
| | TGGCTTCTTTCCAACCTCTCTCCTTCCTCTCCCCAAGTCCTGTTTCTTTGAA | |
| | GCTGGTAACTGAATTTAAGATGATATCTGGTTGGTGTTTAAGGTTTGAGCC | |
| | TCCCAAGGTTCTGTGCATTTTGAAAGGAGATTTCTAAAAATAATTAAGGT | |
| | GCCCTAACTCCTTTCCTCATGATTCCTACTCCGAAACCTGGATGGTTAGGA | |
| | GCCCAGGGCTCCCTGATTTCCAGAGCTATATCCTGTTGGACCTTTGCCAAC | |
| | AGACCTGACACTTAGGGGTTATTGTTATAAATCTAATTCTCTAATATTTTT | |
| | TACATGTTGTTTCACTTTGAATAAGCAAATGAAGAATCAGTTTTCTAATAT | |
| | GACTTTATCCTCAAGCTAGAGACACTAGCCTATTTGGTAAATCACACATT | |
| | ACTTAGGTATATTTATTACTATAACCAGGTTGGAGCTTCCATGTTTAAGCT | |
| | GGGTATATGATGGGTTTTTGTTAAAATGTGCCTTAAAAAGCCTATTACTTC | |
| | AAGAGCAAATGATTCTTTGGGGGAAAGGCAAAAATAATTCTATGACATA | |
| | GGGCCCAAGTTCATGGTAGTAAGTGTACTCTTTGATTAATCACACGCTAA | |
| | TATAGATTACTGCCTCTAACTTTGTAAGTGTGGCAATGACTTCTTAATTAA | |
| | AGAAAGATGCAGGAGTTATGTCTAAGCGTTCAGTTTTTCAAATCTGTGTT | |
| | ATTGGAAATGTCTTCAAGTCATTTTGCATTGTATTTTTGATATGAGAGGCA | |
| | GCTTATTGCGATGTGTATGGCCATGTTTCATTCTCAAATTTAATTCTATAA | |
| | ATACAAATCCTAAATACATGGCTACAGCAACTGCACTGGAACATTTTTGC | |
| | TTGGTTTTAGGGATTGAGAACTTGCCTTGCAGGTTTCCTTCCTCAAAAGGA | |
| | GCAGGGCAGTCCTTTCCCTGTTGAGTCAATTAGAATTTTTACATAGAGGTG | |
| | AGACTGTGAATTATTTTGGTTATTTTCAGTGATGTAGATTAGTGTGAATGA | |
| | CCAGGGTGGAATGTTTTTGAAGGAATATAAAGCAAAAACTGGTTGACATT | |
| | CACAAACTGTTCTTTTGTGAACATATTTTGGACCCTTAAATATGACTAAAA | |
| | TCACAGCAATATTGTTACATACGGGTTATATGCCAACTCTGTTTGAAATAT | |
| | ACTCTGGAAAAACAGCTGAATTGTCTTGGTTATTAAAGTATGGTATGTATT | |
| | CAACTTGTACAGACTGGATGTAATTTGTAATCAGGTATAGTCCATGTTTTA | |
| | CTTTAAGCAGTACATCACTTAATAACCATTGTTAAGCCATTGCTTTCAAGA | |
| | ATGTTAACTGCCAATTTAAAAGCATGTGTCCTAGGTTCATGCTTTGGTAAA | |
| | GCTCTCATTTCAAGTGTATTCATAGCTAAGCTTTCTGGGAGCAGAATTGTC | |
| | TCTTTGGTGAAAAGGAAGTACAGCCTTTCCTGTTTCTGAGGTTGCTTACCA | |
| | TACATGTATGTCACTGTTTCATTGGCCCTGTTACATCCATTTGGTAAAATT | |
| | TATTTGTCCTGATTAACCAGCTCTCATTTTATGGAAATGATGATAAATCTC | |
| | ACTACTTAAATTTAATTTATGCTTTTATTTTTAA | |
| FABP5 | CGCCGCCCAGCGCGGGCCGCCGTTATAAAGCAGCCGCCGGCGCCGGGTG | 199 |
| NM_001444.2 | CCTCACAGCACGCTGCCACGCCGACGCAGACCCCTCTCTGCACGCCAGCC | |
| | CGCCCGCACCCACCATGGCCACAGTTCAGCAGCTGGAAGGAAGATGGCG | |
| | CCTGGTGGACAGCAAAGGCTTTGATGAATACATGAAGGAGCTAGGAGTG | |
| | GGAATAGCTTTGCGAAAAATGGGCGCAATGGCCAAGCCAGATTGTATCAT | |
| | CACTTGTGATGGTAAAAACCTCACCATAAAAACTGAGAGCACTTTGAAAA | |
| | CAACACAGTTTTCTTGTACCCTGGGAGAGAAGTTTGAAGAAACCACAGCT | |
| | GATGGCAGAAAAACTCAGACTGTCTGCAACTTTACAGATGGTGCATTGGT | |
| | TCAGCATCAGGAGTGGGATGGGAAGGAAAGCACAATAACAAGAAAATTG | |
| | AAAGATGGGAAATTAGTGGTGGAGTGTGTCATGAACAATGTCACCTGTAC | |
| | TCGGATCTATGAAAAAGTAGAATAAAAATTCCATCATCACTTTGGACAGG | |
| | AGTTAATTAAGAGAATGACCAAGCTCAGTTCAATGAGCAAATCTCCATAC | |
| | TGTTTCTTTCTTTTTTTTTTCATTACTGTGTTCAATTATCTTTATCATAAACA | |
| | TTTTACATGCAGCTATTTCAAAGTGTGTTGGATTAATTAGGATCATCCCTT | |
| | TGGTTAATAAATAAATGTGTTTGTGCTAATAAAAAAAAAAAAAAAAAAA | |
| | AA | |
| UCHL1 | AGTGCGTCTGGCCGGCGCTTTATAGCTGCAGCCTGGGCGGCTCCGCTAGC | 200 |
| NM_004181.4 | TGTTTTTCGTCTTCCCTAGGCTATTTCTGCCGGGCGCTCCGCGAAGATGCA | |
| | GCTCAAGCCGATGGAGATCAACCCCGAGATGCTGAACAAAGTGCTGTCCC | |
| | GGCTGGGGGTCGCCGGCCAGTGGCGCTTCGTGGACGTGCTGGGGCTGGAA | |
| | GAGGAGTCTCTGGGCTCGGTGCCAGCGCCTGCCTGCGCGCTGCTGCTGCT | |
| | GTTTCCCCTCACGGCCCAGCATGAGAACTTCAGGAAAAAGCAGATTGAAG | |
| | AGCTGAAGGGACAAGAAGTTAGTCCTAAAGTGTACTTCATGAAGCAGAC | |
| | CATTGGGAATTCCTGTGGCACAATCGGACTTATTCACGCAGTGGCCAATA | |
| | ATCAAGACAAACTGGGATTTGAGGATGGATCAGTTCTGAAACAGTTTCTT | |
| | TCTGAAACAGAGAAAATGTCCCCTGAAGACAGAGCAAAATGCTTTGAAA | |
| | AGAATGAGGCCATACAGGCAGCCCATGATGCCGTGGCACAGGAAGGCCA | |
| | ATGTCGGGTAGATGACAAGGTGAATTTCCATTTTATTCTGTTTAACAACGT | |
| | GGATGGCCACCTCTATGAACTTGATGGACGAATGCCTTTTCCGGTGAACC | |
| | ATGGCGCCAGTTCAGAGGACACCCTGCTGAAGGACGCTGCCAAGGTCTGC | |
| | AGAGAATTCACCGAGCGTGAGCAAGGAGAAGTCCGCTTCTCTGCCGTGGC | |
| | TCTCTGCAAGGCAGCCTAATGCTCTGTGGGAGGGACTTTGCTGATTTCCCC | |
| | TCTTCCCTTCAACATGAAAATATATACCCCCCCATGCAGTCTAAAATGCTT | |
| | CAGTACTTGTGAAACACAGCTGTTCTTCTGTTCTGCAGACACGCCTTCCCC | |
| | TCAGCCACACCCAGGCACTTAAGCACAAGCAGAGTGCACAGCTGTCCACT | |
| | GGGCCATTGTGGTGTGAGCTTCAGATGGTGAAGCATTCTCCCCAGTGTAT | |
| | GTCTTGTATCCGATATCTAACGCTTTAAATGGCTACTTTGGTTTCTGTCTGT | |
| | AAGTTAAGACCTTGGATGTGGTTTAATTGTTTGTCCTCAAAAGGAATAAA | |
| | ACTTTTCTGCTGATAAGATAAAAAAAAAAAAAAAAAA | |
| GAL | ATATAGCAGCGGCGGCGGTGGCGGCGGCCACACCGGGCGGCGGACACGT | 201 |
| NM_015973.3 | GGAGGGACCCGGCCCGCGCCTTCTGCCCCTGCTGCCGGCCGCGCCATGCG | |
| | GTGAGCGCCCCAGGCCGCCAGAGCCCACCCGACCCGGCCCGACGCCCGG | |
| | ACCTGCCGCCCAGACCCGCCACCGCACCCGGACCCCGACGCTCCGAACCC | |
| | GGGCGCAGCCGCAGCTCAAGATGGCCCGAGGCAGCGCCCTCCTGCTCGCC | |
| | TCCCTCCTCCTCGCCGCGGCCCTTTCTGCCTCTGCGGGGCTCTGGTCGCCG | |
| | GCCAAGGAAAAACGAGGCTGGACCCTGAACAGCGCGGGCTACCTGCTGG | |
| | GCCCACATGCCGTTGGCAACCACAGGTCATTCAGCGACAAGAATGGCCTC | |
| | ACCAGCAAGCGGGAGCTGCGGCCCGAAGATGACATGAAACCAGGAAGCT | |
| | TTGACAGGTCCATACCTGAAAACAATATCATGCGCACAATCATTGAGTTT | |
| | CTGTCTTTCTTGCATCTCAAAGAGGCCGGTGCCCTCGACCGCCTCCTGGAT | |
| | CTCCCCGCCGCAGCCTCCTCAGAAGACATCGAGCGGTCCTGAGAGCCTCC | |
| | TGGGCATGTTTGTCTGTGTGCTGTAACCTGAAGTCAAACCTTAAGATAAT | |
| | GGATAATCTTCGGCCAATTTATGCAGAGTCAGCCATTCCTGTTCTCTTTGC | |
| | CTTGATGTTGTGTTGTTATCATTTAAGATTTTTTTTTTTTGGTAATTATTTT | |
| | GAGTGGCAAAATAAAGAATAGCAATTA | |
| PLOD | CCACCATATCGGTCCCGTATTTCACATTGATAAGGTCCTGTTTCATTTCTC | 202 |
| L06419.1 | GTGACATTGGGTAGAATGAGGATCCTGTTTTCAATGGGTCGCTTTACCCTG | |
| | GGACTGACAGGGAGGCTCTGACCATTTAGCCACCAAATGTAGGTGTAGTT | |
| | CTCACTCTTAGGTTCACCCCGCGGCCGATCGTCCCCCATACCTCGGCCATG | |
| | CGGCCCCTGCTGCTACTGGCCCTGCTGGGCTGGCTGCTGCTGGCCGAAGC | |
| | GAAGGGCGACGCCAAGCCGGAGGACAACCTTTTAGTCCTCACGGTGGCC | |
| | ACTAAGGAGACCGAGGGATTCCGTCGCTTCAAGCGCTCAGCTCAGTTCTT | |
| | CAACTACAAGATCCAGGCGCTTGGCCTAGGGGAGGACTGGAATGTGGAG | |
| | AAGGGGACGTCGGCAGGTGGAGGGCAGAAGGTCCGGCTGCTGAAGAAAG | |
| | CTCTGGAGAAGCACGCAGACAAGGAGGATCTGGTCATTCTCTTCACAGAC | |
| | AGCTATGACGTGCTGTTTGCATCGGGGCCCCGGGAGCTCCTGAAGAAGTT | |
| | CCGGCAGGCCAGGAGCCAGGTGGTCTTCTCTGCTGAGGAGCTCATCTACC | |
| | CAGACCGCAGGCTGGAGACCAAGTATCCGGTGGTGTCCGATGGCAAGAG | |
| | GTTCCTGGGCTCTGGAGGCTTCATCGGTTATGCCCCCAACCTCAGCAAACT | |
| | GGTGGCCGAGTGGGAGGGCCAGGACAGCGACAGCGATCAGCTGTTTTAC | |
| | ACCAAGATCTTCTTGGACCCGGAGAAGAGGGAGCAGATCAATATCACCCT | |
| | GGACCACCGCTGCCGTATCTTCCAGAACCTGGATGGAGCCTTGGATGAGG | |
| | TCGTGCTCAAGTTTGAAATGGGCCATGTGAGAGCGAGGAACCTGGCCTAT | |
| | GACACCCTCCCGGTCCTGATCCATGGCAACGGGCCAACCAAGCTGCAGTT | |
| | GAACTACCTGGGCAACTACATCCCGCGCTTCTGGACCTTCGAAACAGGCT | |
| | GCACCGTGTGTGACGAAGGCTTGCGCAGCCTCAAGGGCATTGGGGATGA | |
| | AGCTCTGCCCACGGTCCTGGTCGGCGTGTTCATCGAACAGCCCACGCCGT | |
| | TTGTGTCCCTGTTCTTCCAGCGGCTCCTGCGGCTCCACTACCCCCAGAAAC | |
| | ACATGCGACTTTTCATCCACAACCACGAGCAGCACCACAAGGCTCAGGTG | |
| | GAAGAGTTCCTGGCACAGCATGGCAGCGAGTACCAGTCTGTGAAGCTGGT | |
| | GGGCCCTGAGGTGCGGATGGCGAATGCAGATGCCAGGAACATGGGCGCA | |
| | GACCTGTGCCGGCAGGACCGCAGCTGCACCTACTACTTCAGCGTGGATGC | |
| | TGACGTGGCCCTGACCGAGCCCAACAGCCTGCGGCTGCTGATCCAACAGA | |
| | ACAAGAATGTCATTGCCCCGCTGATGACCCGGCATGGGAGGCTGTGGTCG | |
| | AACTTCTGGGGGGCTCTCAGTGCAGATGGCTACTATGCCCGTTCCGAGGA | |
| | CTACGTGGACATTGTGCAGGGGCGGCGTGTTGGTGTCTGGAATGTGCCCT | |
| | ATATTTCAAACATCTACTTGATCAAGGGCAGTGCCCTGCGGGGTGAGCTG | |
| | CAGTCCTCAGATCTCTTCCACCACAGCAAGCTGGACCCCGACATGGCCTT | |
| | CTGTGCCAACATCCGGCAGCAGGATGTGTTCATGTTCCTGACCAACCGGC | |
| | ACACCCTTGGCCATCTGCTCTCCCTAGACAGCTACCGCACCACCCACCTGC | |
| | ACAACGACCTCTGGGAGGTGTTCAGCAACCCCGAGGACTGGAAGGAGAA | |
| | GTACATCCACCAGAACTACACCAAAGCCCTGGCAGGGAAGCTGGTGGAG | |
| | ACGCCCTGCCCGGATGTCTATTGGTTCCCCATCTTCACGGAGGTGGCCTGT | |
| | GATGAGCTGGTGGAGGAGATGGAGCACTTTGGCCAGTGGTCTCTGGGCAA | |
| | CAACAAGGACAACCGCATCCAGGGTGGCTACGAGAACGTGCCGACTATT | |
| | GACATCCACATGAACCAGATCGGCTTTGAGCGGGAGTGGCACAAATTCCT | |
| | GCTGGAGTACATTGCGCCCATGACGGAGAAGCTCTACCCCGGCTACTACA | |
| | CCAGGGCCCAGTTTGACCTGGCCTTTGTCGTCCGCTACAAGCCTGATGAG | |
| | CAGCCCTCACTGATGCCACACCATGATGCCTCCACCTTCACCATCAACATC | |
| | GCCCTGAACCGAGTCGGGGTGGATTACGAGGGCGGGGGCTGTCGGTTCCT | |
| | GCGCTACAACTGTTCCATCCGAGCCCCAAGGAAGGGCTGGACCCTCATGC | |
| | ACCCTGGACGACTCACGCATTACCATGAGGGGCTCCCCACCACCAGGGGC | |
| | ACCCGCTACATCGCAGTCTCCTTCGTCGATCCCTAATTGGCCAGGCCTGAC | |
| | CCTCTTGGACCTTTCTTCTTTGCCGACAACCACTGCCCAGCAGCCTCTGGG | |
| | ACCTCGGGGTCCCAGGGAACCCAGTCCAGCCTCCTGGCTGTTGACTTCCC | |
| | ATTGCTCTTGGAGCCACCAATCAAAGAGATTCAAAGAGATTCCTGCAGGC | |
| | CAGAGGCCGGAACACACCTTTATGGCTGGGGCTCTCCGTGGTGTTCTGGA | |
| | CCCAGCCCCTGGAGACACCATTCACTTTTACTGCTTTGTAGTGACTCGTGC | |
| | TCTCCAACCTGTCTTCCTGAAAAACCAAGGCCCCCTTCCCCCACCTCTTCC | |
| | ATGGGGTGAGACTTGAGCAGAACAGGGGCTTCCCCAAGTTGCCCAGAAA | |
| | GACTGTCTGGGTGAGAAGCCATGGCCAGAGCTTCTCCCAGGCACAGGTGT | |
| | TGCACCAGGGACTTCTGCTTCAAGTTTTGGGGTAAAGACACCTGGATCAG | |
| | ACTCCAAGGGCTGCCCTGAGTCTGGGACTTCTGCCTCCATGGCTGGTCAT | |
| | GAGAGCAAACCGTAGTCCCCTGGAGACAGCCACTCCAGAGAACCTCTTGG | |
| | GAGACAGAAGAGGCATCTGTGCACAGCTCGATCTTCTACTTGCCTGTGGG | |
| | GAGGGGAGTGACAGGTCCACACACCACACTGGGTCACCCTGTCCTGGATG | |
| | CCTCTGAAGAGAGGGACAGACCGTCAGAAACTGGAGAGTTTCTATTAAA | |
| | GGTCATTTAAACCAC | |
| DDIT4 | AGGGCGCAGCAGGCCAAGGGGGAGGTGCGAGCGTGGACCTGGGACGGGT | 203 |
| NM_019058.2 | CTGGGCGGCTCTCGGTGGTTGGCACGGGTTCGCACACCCATTCAAGCGGC | |
| | AGGACGCACTTGTCTTAGCAGTTCTCGCTGACCGCGCTAGCTGCGGCTTCT | |
| | ACGCTCCGGCACTCTGAGTTCATCAGCAAACGCCCTGGCGTCTGTCCTCA | |
| | CCATGCCTAGCCTTTGGGACCGCTTCTCGTCGTCGTCCACCTCCTCTTCGC | |
| | CCTCGTCCTTGCCCCGAACTCCCACCCCAGATCGGCCGCCGCGCTCAGCCT | |
| | GGGGGTCGGCGACCCGGGAGGAGGGGTTTGACCGCTCCACGAGCCTGGA | |
| | GAGCTCGGACTGCGAGTCCCTGGACAGCAGCAACAGTGGCTTCGGGCCG | |
| | GAGGAAGACACGGCTTACCTGGATGGGGTGTCGTTGCCCGACTTCGAGCT | |
| | GCTCAGTGACCCTGAGGATGAACACTTGTGTGCCAACCTGATGCAGCTGC | |
| | TGCAGGAGAGCCTGGCCCAGGCGCGGCTGGGCTCTCGACGCCCTGCGCGC | |
| | CTGCTGATGCCTAGCCAGTTGGTAAGCCAGGTGGGCAAAGAACTACTGCG | |
| | CCTGGCCTACAGCGAGCCGTGCGGCCTGCGGGGGGCGCTGCTGGACGTCT | |
| | GCGTGGAGCAGGGCAAGAGCTGCCACAGCGTGGGCCAGCTGGCACTCGA | |
| | CCCCAGCCTGGTGCCCACCTTCCAGCTGACCCTCGTGCTGCGCCTGGACTC | |
| | ACGACTCTGGCCCAAGATCCAGGGGCTGTTTAGCTCCGCCAACTCTCCCTT | |
| | CCTCCCTGGCTTCAGCCAGTCCCTGACGCTGAGCACTGGCTTCCGAGTCAT | |
| | CAAGAAGAAGCTGTACAGCTCGGAACAGCTGCTCATTGAGGAGTGTTGA | |
| | ACTTCAACCTGAGGGGGCCGACAGTGCCCTCCAAGACAGAGACGACTGA | |
| | ACTTTTGGGGTGGAGACTAGAGGCAGGAGCTGAGGGACTGATTCCTGTGG | |
| | TTGGAAAACTGAGGCAGCCACCTAAGGTGGAGGTGGGGGAATAGTGTTT | |
| | CCCAGGAAGCTCATTGAGTTGTGTGCGGGTGGCTGTGCATTGGGGACACA | |
| | TACCCCTCAGTACTGTAGCATGAAACAAAGGCTTAGGGGCCAACAAGGCT | |
| | TCCAGCTGGATGTGTGTGTAGCATGTACCTTATTATTTTTGTTACTGACAG | |
| | TTAACAGTGGTGTGACATCCAGAGAGCAGCTGGGCTGCTCCCGCCCCAGC | |
| | CCGGCCCAGGGTGAAGGAAGAGGCACGTGCTCCTCAGAGCAGCCGGAGG | |
| | GAGGGGGGAGGTCGGAGGTCGTGGAGGTGGTTTGTGTATCTTACTGGTCT | |
| | GAAGGGACCAAGTGTGTTTGTTGTTTGTTTTGTATCTTGTTTTTCTGATCG | |
| | GAGCATCACTACTGACCTGTTGTAGGCAGCTATCTTACAGACGCATGAAT | |
| | GTAAGAGTAGGAAGGGGTGGGTGTCAGGGATCACTTGGGATCTTTGACAC | |
| | TTGAAAAATTACACCTGGCAGCTGCGTTTAAGCCTTCCCCCATCGTGTACT | |
| | GCAGAGTTGAGCTGGCAGGGGAGGGGCTGAGAGGGTGGGGGCTGGAACC | |
| | CCTCCCCGGGAGGAGTGCCATCTGGGTCTTCCATCTAGAACTGTTTACATG | |
| | AAGATAAGATACTCACTGTTCATGAATACACTTGATGTTCAAGTATTAAG | |
| | ACCTATGCAATATTTTTTACTTTTCTAATAAACATGTTTGTTAAAACAGTT | |
| VEGF | TCGGGCCTCCGAAACCATGAACTTTCTGCTGTCTTGGGTGCATTGGAGCCT | 204 |
| AY047581.1 | TGCCTTGCTGCTCTACCTCCACCATGCCAAGTGGTCCCAGGCTGCACCCAT | |
| | GGCAGAAGGAGGGGGGCAGAATCATCACGAAGTGGTGAAGTTCATGGAT | |
| | GTCTATCAGCGCAGCTACTGCCATCCAATCGAGACCCTGGTGGACATCTT | |
| | CCAGGAGTACCCTGATGAGATCGAGTACATCTTCAAGCCATCCTGTGTGC | |
| | CCCTGATGCGATGCGGGGGCTGCTGCAATGACGAGGGCCTGGAGTGTGTG | |
| | CCCACTGAGGAGTCCAACATCACCATGCAGATTATGCGGATCAAACCTCA | |
| | CCAAGGCCAGCACATAGGAGAGATGAGCTTCCTACAGCACAACAAATGT | |
| | GAATGCAGACCAAAGAAAGATAGAGCAAGACAAGAAAATCCCTGTGGGC | |
| | CTTGCTCAGAGCGGAGAAAGCATTTGTTTGTACAAGATCCGCAGACGTGT | |
| | AAATGTTCCTGCAAAAACACAGACTCGCGTTGCAAGGCGAGGCAGCTTGA | |
| | GTTAAACGAACGTACTTGCAGATGTGACAAGCCGAGGCGGTGAGCCGGG | |
| | CAGGAGGAAGGAGCCTCCCTCAGGGTTTCGGGAACCAGATCT | |
| ADM | CTGGATAGAACAGCTCAAGCCTTGCCACTTCGGGCTTCTCACTGCAGCTG | 205 |
| NM_001124.1 | GGCTTGGACTTCGGAGTTTTGCCATTGCCAGTGGGACGTCTGAGACTTTCT | |
| | CCTTCAAGTACTTGGCAGATCACTCTCTTAGCAGGGTCTGCGCTTCGCAGC | |
| | CGGGATGAAGCTGGTTTCCGTCGCCCTGATGTACCTGGGTTCGCTCGCCTT | |
| | CCTAGGCGCTGACACCGCTCGGTTGGATGTCGCGTCGGAGTTTCGAAAGA | |
| | AGTGGAATAAGTGGGCTCTGAGTCGTGGGAAGAGGGAACTGCGGATGTC | |
| | CAGCAGCTACCCCACCGGGCTCGCTGACGTGAAGGCCGGGCCTGCCCAGA | |
| | CCCTTATTCGGCCCCAGGACATGAAGGGTGCCTCTCGAAGCCCCGAAGAC | |
| | AGCAGTCCGGATGCCGCCCGCATCCGAGTCAAGCGCTACCGCCAGAGCAT | |
| | GAACAACTTCCAGGGCCTCCGGAGCTTTGGCTGCCGCTTCGGGACGTGCA | |
| | CGGTGCAGAAGCTGGCACACCAGATCTACCAGTTCACAGATAAGGACAA | |
| | GGACAACGTCGCCCCCAGGAGCAAGATCAGCCCCCAGGGCTACGGCCGC | |
| | CGGCGCCGGCGCTCCCTGCCCGAGGCCGGCCCGGGTCGGACTCTGGTGTC | |
| | TTCTAAGCCACAAGCACACGGGGCTCCAGCCCCCCCGAGTGGAAGTGCTC | |
| | CCCACTTTCTTTAGGATTTAGGCGCCCATGGTACAAGGAATAGTCGCGCA | |
| | AGCATCCCGCTGGTGCCTCCCGGGACGAAGGACTTCCCGAGCGGTGTGGG | |
| | GACCGGGCTCTGACAGCCCTGCGGAGACCCTGAGTCCGGGAGGCACCGTC | |
| | CGGCGGCGAGCTCTGGCTTTGCAAGGGCCCCTCCTTCTGGGGGCTTCGCTT | |
| | CCTTAGCCTTGCTCAGGTGCAAGTGCCCCAGGGGGCGGGGTGCAGAAGA | |
| | ATCCGAGTGTTTGCCAGGCTTAAGGAGAGGAGAAACTGAGAAATGAATG | |
| | CTGAGACCCCCGGAGCAGGGGTCTGAGCCACAGCCGTGCTCGCCCACAA | |
| | ACTGATTTCTCACGGCGTGTCACCCCACCAGGGCGCAAGCCTCACTATTA | |
| | CTTGAACTTTCCAAAACCTAAAGAGGAAAAGTGCAATGCGTGTTGTACAT | |
| | ACAGAGGTAACTATCAATATTTAAGTTTGTTGCTGTCAAGATTTTTTTTGT | |
| | AACTTCAAATATAGAGATATTTTTGTACGTTATATATTGTATTAAGGGCAT | |
| | TTTAAAAGCAATTATATTGTCCTCCCCTATTTTAAGACGTGAATGTCTCAG | |
| | CGAGGTGTAAAGTTGTTCGCCGCGTGGAATGTGAGTGTGTTTGTGTGCAT | |
| | GAAAGAGAAAGACTGATTACCTCCTGTGTGGAAGAAGGAAACACCGAGT | |
| | CTCTGTATAATCTATTTACATAAAATGGGTGATATGCGAACAGCAAACC | |
| ANGPTL4 | GACTGTGATCCGATTCTTTCCAGCGGCTTCTGCAACCAAGCGGGTCTTACC | 206 |
| BCO23647.2 | CCCGGTCCTCCGCGTCTCCAGTCCTCGCACCTGGAACCCCAACGTCCCCG | |
| | AGAGTCCCCGAATCCCCGCTCCCAGGCTACCTAAGAGGATGAGCGGTGCT | |
| | CCGACGGCCGGGGCAGCCCTGATGCTCTGCGCCGCCACCGCCGTGCTACT | |
| | GAGCGCTCAGGGCGGACCCGTGCAGTCCAAGTCGCCGCGCTTTGCGTCCT | |
| | GGGACGAGATGAATGTCCTGGCGCACGGACTCCTGCAGCTCGGCCAGGG | |
| | GCTGCGCGAACACGCGGAGCGCACCCGCAGTCAGCTGAGCGCGCTGGAG | |
| | CGGCGCCTGAGCGCGTGCGGGTCCGCCTGTCAGGGAACCGAGGGGTCCA | |
| | CCGACCTCCCGTTAGCCCCTGAGAGCCGGGTGGACCCTGAGGTCCTTCAC | |
| | AGCCTGCAGACACAACTCAAGGCTCAGAACAGCAGGATCCAGCAACTCTT | |
| | CCACAAGGTGGCCCAGCAGCAGCGGCACCTGGAGAAGCAGCACCTGCGA | |
| | ATTCAGCATCTGCAAAGCCAGTTTGGCCTCCTGGACCACAAGCACCTAGA | |
| | CCATGAGGTGGCCAAGCCTGCCCGAAGAAAGAGGCTGCCCGAGATGGCC | |
| | CAGCCAGTTGACCCGGCTCACAATGTCAGCCGCCTGCACCGGCTGCCCAG | |
| | GGATTGCCAGGAGCTGTTCCAGGTTGGGGAGAGGCAGAGTGGACTATTTG | |
| | AAATCCAGCCTCAGGGGTCTCCGCCATTTTTGGTGAACTGCAAGATGACC | |
| | TCAGATGGAGGCTGGACAGTAATTCAGAGGCGCCACGATGGCTCAGTGG | |
| | ACTTCAACCGGCCCTGGGAAGCCTACAAGGCGGGGTTTGGGGATCCCCAC | |
| | GGCGAGTTCTGGCTGGGTCTGGAGAAGGTGCATAGCATCACGGGGGACC | |
| | GCAACAGCCGCCTGGCCGTGCAGCTGCGGGACTGGGATGGCAACGCCGA | |
| | GTTGCTGCAGTTCTCCGTGCACCTGGGTGGCGAGGACACGGCCTATAGCC | |
| | TGCAGCTCACTGCACCCGTGGCCGGCCAGCTGGGCGCCACCACCGTCCCA | |
| | CCCAGCGGCCTCTCCGTACCCTTCTCCACTTGGGACCAGGATCACGACCTC | |
| | CGCAGGGACAAGAACTGCGCCAAGAGCCTCTCTGGAGGCTGGTGGTTTGG | |
| | CACCTGCAGCCATTCCAACCTCAACGGCCAGTACTTCCGCTCCATCCCAC | |
| | AGCAGCGGCAGAAGCTTAAGAAGGGAATCTTCTGGAAGACCTGGCGGGG | |
| | CCGCTACTACCCACTGCAGGCCACCACCATGTTGATCCAGCCCATGGCAG | |
| | CAGAGGCAGCCTCCTAGCGTCCTGGCTGGGCCTGGTCCCAGGCCCACGAA | |
| | AGACGGTGACTCTTGGCTCTGCCCGAGGATGTGGCCGTTCCCTGCCTGGG | |
| | CAGGGGCTCCAAGGAGGGGCCATCTGGAAACTTGTGGACAGAGAAGAAG | |
| | ACCACGACTGGAGAAGCCCCCTTTCTGAGTGCAGGGGGGCTGCATGCGTT | |
| | GCCTCCTGAGATCGAGGCTGCAGGATATGCTCAGACTCTAGAGGCGTGGA | |
| | CCAAGGGGCATGGAGCTTCACTCCTTGCTGGCCAGGGAGTTGGGGACTCA | |
| | GAGGGACCACTTGGGGCCAGCCAGACTGGCCTCAATGGCGGACTCAGTC | |
| | ACATTGACTGACGGGGACCAGGGCTTGTGTGGGTCGAGAGCGCCCTCATG | |
| | GTGCTGGTGCTGTTGTGTGTAGGTCCCCTGGGGACACAAGCAGGCGCCAA | |
| | TGGTATCTGGGCGGAGCTCACAGAGTTCTTGGAATAAAAGCAACCTCAGA | |
| | AAAAAAAAAAAAAAAAAA | |
| NDRG1 | ATGTCTCGGGAGATGCAGGATGTAGACCTCGCTGAGGTGAAGCCTTTGGT | 207 |
| CR456842.1 | GGAGAAAGGGGAGACCATCACCGGCCTCCTGCAAGAGTTTGATGTCCAG | |
| | GAGCAGGACATCGAGACTTTACATGGCTCTGTTCACGTCACGCTGTGTGG | |
| | GACTCCCAAGGGAAACCGGCCTGTCATCCTCACCTACCATGACATCGGCA | |
| | TGAACCACAAAACCTGCTACAACCCCCTCTTCAACTACGAGGACATGCAG | |
| | GAGATCACCCAGCACTTTGCCGTCTGCCACGTGGACGCCCCTGGCCAGCA | |
| | GGACGGCGCAGCCTCCTTCCCCGCAGGGTACATGTACCCCTCCATGGATC | |
| | AGCTGGCTGAAATGCTTCCTGGAGTCCTTCAACAGTTTGGGCTGAAAAGC | |
| | ATTATTGGCATGGGAACAGGAGCAGGCGCCTACATCCTAACTCGATTTGC | |
| | TCTAAACAACCCTGAGATGGTGGAGGGCCTTGTCCTTATCAACGTGAACC | |
| | CTTGTGCGGAAGGCTGGATGGACTGGGCCGCCTCCAAGATCTCAGGATGG | |
| | ACCCAAGCTCTGCCGGACATGGTGGTGTCCCACCTTTTTGGGAAGGAAGA | |
| | AATGCAGAGTAACGTGGAAGTGGTCCACACCTACCGCCAGCACATTGTGA | |
| | ATGACATGAACCCCGGCAACCTGCACCTGTTCATCAATGCCTACAACAGC | |
| | CGGCGCGACCTGGAGATTGAGCGACCAATGCCGGGAACCCACACAGTCA | |
| | CCCTGCAGTGCCCTGCTCTGTTGGTGGTTGGGGACAGCTCGCCTGCAGTG | |
| | GATGCCGTGGTGGAGTGCAACTCAAAATTGGACCCAACAAAGACCACTCT | |
| | CCTCAAGATGGCGGACTGTGGCGGCCTCCCGCAGATCTCCCAGCCGGCCA | |
| | AGCTCGCTGAGGCCTTCAAGTACTTCGTGCAGGGCATGGGATACATGCCC | |
| | TCGGCTAGCATGACCCGCCTGATGCGGTCCCGCACAGCCTCTGGTTCCAG | |
| | CGTCACTTCTCTGGATGGCACCCGCAGCCGCTCCCACACCAGCGAGGGCA | |
| | CCCGAAGCCGCTCCCACACCAGCGAGGGCACCCGCAGCCGCTCGCACACC | |
| | AGCGAGGGGGCCCACCTGGACATCACCCCCAACTCGGGTGCTGCTGGGA | |
| | ACAGCGCCGGGCCCAAGTCCATGGAGGTCTCCTGTTAA | |
| NP | ATAAGCCAGAGCCTAGACCAGTGAGCCAACTGTGCGAACCAGACCCGGC | 208 |
| NM_000270.3 | AGCCTTGCTCAGTTCAGCATAGCGGAGCGGATCCGATCGGATCGGAGCGG | |
| | ATCGGAGCACACCGGAGCAGGCTCATCGAGAAGGCGTCTGCGAGACCAT | |
| | GGAGAACGGATACACCTATGAAGATTATAAGAACACTGCAGAATGGCTT | |
| | CTGTCTCACACTAAGCACCGACCTCAAGTTGCAATAATCTGTGGTTCTGG | |
| | ATTAGGAGGTCTGACTGATAAATTAACTCAGGCCCAGATCTTTGACTACG | |
| | GTGAAATCCCCAACTTTCCCCGAAGTACAGTGCCAGGTCATGCTGGCCGA | |
| | CTGGTGTTTGGGTTCCTGAATGGCAGGGCCTGTGTGATGATGCAGGGCAG | |
| | GTTCCACATGTATGAAGGGTACCCACTCTGGAAGGTGACATTCCCAGTGA | |
| | GGGTTTTCCACCTTCTGGGTGTGGACACCCTGGTAGTCACCAATGCAGCA | |
| | GGAGGGCTGAACCCCAAGTTTGAGGTTGGAGATATCATGCTGATCCGTGA | |
| | CCATATCAACCTACCTGGTTTCAGTGGTCAGAACCCTCTCAGAGGGCCCA | |
| | ATGATGAAAGGTTTGGAGATCGTTTCCCTGCCATGTCTGATGCCTACGAC | |
| | CGGACTATGAGGCAGAGGGCTCTCAGTACCTGGAAACAAATGGGGGAGC | |
| | AACGTGAGCTACAGGAAGGCACCTATGTGATGGTGGCAGGCCCCAGCTTT | |
| | GAGACTGTGGCAGAATGTCGTGTGCTGCAGAAGCTGGGAGCAGACGCTG | |
| | TTGGCATGAGTACAGTACCAGAAGTTATCGTTGCACGGCACTGTGGACTT | |
| | CGAGTCTTTGGCTTCTCACTCATCACTAACAAGGTCATCATGGATTATGAA | |
| | AGCCTGGAGAAGGCCAACCATGAAGAAGTCTTAGCAGCTGGCAAACAAG | |
| | CTGCACAGAAATTGGAACAGTTTGTCTCCATTCTTATGGCCAGCATTCCAC | |
| | TCCCTGACAAAGCCAGTTGACCTGCCTTGGAGTCGTCTGGCATCTCCCAC | |
| | ACAAGACCCAAGTAGCTGCTACCTTCTTTGGCCCCTTGCTGGAGTCATGTG | |
| | CCTCTGTCCTTAGGTTGTAGCAGAAAGGAAAAGATTCCTGTCCTTCACCTT | |
| | TCCCACTTTCTTCTACCAGACCCTTCTGGTGCCAGATCCTCTTCTCAAAGC | |
| | TGGGATTACAGGTGTGAGCATAGTGAGACCTTGGCGCTACAAAATAAAGC | |
| | TGTTCTCATTCCTGTTCTTTCTTACACAAGAGCTGGAGCCCGTGCCCTACC | |
| | ACACATCTGTGGAGATGCCCAGGATTTGACTCGGGCCTTAGAACTTTGCA | |
| | TAGCAGCTGCTACTAGCTCTTTGAGATAATACATTCCGAGGGGCTCAGTT | |
| | CTGCCTTATCTAAATCACCAGAGACCAAACAAGGACTAATCCAATACCTC | |
| | TTGGATTTTATTTAATGTCATAATGTTGTCAGAATAAAGAGAAAGATGAA | |
| | ATAATTTCATTTTTTTGTGTAACTTGGTATGGGGCTGGGGCACAGACCAAG | |
| | ATTGACATGAAAGGATGTGAGATCGCATGTCTTGTGTGACTATCTGCTTCT | |
| | CAGACAAGCAGTTAGGAACTGAGATGAGATAGTATGTGAGGGCAGCAAA | |
| | GGATGAAGAAGGGCAAAATGATGAAAGGTGAGGTGGAAAGAGGTTATGA | |
| | GATGGTAAAGAAAAGTTAACTTCTGGCACTTGATTGCCACTTCTGTCAGG | |
| | CTGGTCCTGCCTCTCTCCCTTGCCTTCTGATTGTTTCATTTCCTGTTTATTT | |
| | GATCATATCTGAATTAGTTCACTGGTTAGCCTCTTCCTTAGTTCCCACTTC | |
| | CTTACCAAAGCCCTAATTATATTTCCTCTTGTTTGCCTTTTCTCTCCTACTC | |
| | TTCTCTAACATCTGCAGCCACACTCTCCATTCACTCCATGCTGACAAGGCA | |
| | GTGGCAAACACTTTTCTCTGCTGCCAGCCACTCCACTGTTGACTGGATTGC | |
| | TGCCAGCCCCAGGCAAACCTGTGAAGTTGTTTCATACTCTGCTTCTCTTTG | |
| | AGTGCCTCCTTCTCTCCTTCCTCTTCCTTTCTGGGCTCCAGTCTTTCTCTTC | |
| | ACTTGTGCTTGTCAGAACCTCCCTGTGATACTGCCTCCAGGCATTTCCCCC | |
| | ATGTTGGCTCACCGCACTATTATCTTTGCTTATCAACTTGCATTCAGCTGG | |
| | CTGGCATGTTTCAAAACCACACTGCCCTCCCAGGCCTGTGTGCCTTTTGAG | |
| | AAAGACCAGTGCTGGATGAGCCTCTAGTAATGACAACATTTTAGTTGTTA | |
| | GTGGTATAATACGGAAGAGATATTTTGCACAGGCTGCTTTGGAGAACTTT | |
| | CAAATTATCCTTTGTTTGGTAACTGACCTACTTAACTGCCCAATACAAAGA | |
| | AAAAGCAAAAAAAAAAAAAAAAAA | |
| SLC16A3 | GAATTCGCCCTTCAGGTGAGGCGGAACCAACCCTCCTGGCCATGGGAGGG | 209 |
| BC112269.1 | GCCGTGGTGGACGAGGGCCCCACAGGCGTCAAGGCCCCTGACGGCGGCT | |
| | GGGGCTGGGCCGTGCTCTTCGGCTGTTTCGTCATCACTGGCTTCTCCTACG | |
| | CCTTCCCCAAGGCCGTCAGTGTCTTCTTCAAGGAGCTCATACAGGAGTTTG | |
| | GGATCGGCTACAGCGACACAGCCTGGATCTCCTCCATCCTGCTGGCCATG | |
| | CTCTACGGGACAGGTCCGCTCTGCAGTGTGTGCGTGAACCGCTTTGGCTG | |
| | CCGGCCCGTCATGCTTGTGGGGGGTCTCTTTGCGTCGCTGGGCATGGTGG | |
| | CTGCGTCCTTTTGCCGGAGCATCATCCAGGTCTACCTCACCACTGGGGTCA | |
| | TCACGGGGTTGGGTTTGGCACTCAACTTCCAGCCCTCGCTCATCATGCTGA | |
| | ACCGCTACTTCAGCAAGCGGCGCCCCATGGCCAACGGGCTGGCGGCAGC | |
| | AGGTAGCCCTGTCTTCCTGTGTGCCCTGAGCCCGCTGGGGCAGCTGCTGC | |
| | AGGACCGCTACGGCTGGCGGGGCGGCTTCCTCATCCTGGGCGGCCTGCTG | |
| | CTCAACTGCTGCGTGTGTGCCGCACTCATGAGGCCCCTGGTGGTCACGGC | |
| | CCAGCCGGGCTCGGGGCCGCCGCGACCCTCCCGGCGCCTGCTAGACCTGA | |
| | GCGTCTTCCGGGACCGCGGCTTTGTGCTTTACGCCGTGGCCGCCTCGGTCA | |
| | TGGTGCTGGGGCTCTTCGTCCCGCCCGTGTTCGTGGTGAGCTACGCCAAG | |
| | GACCTGGGCGTGCCCGACACCAAGGCCGCCTTCCTGCTCACCATCCTGGG | |
| | CTTCATTGACATCTTCGCGCGGCCGGCCGCGGGCTTCGTGGCGGGGCTTG | |
| | GGAAGGTGCGGCCCTACTCCGTCTACCTCTTCAGCTTCTCCATGTTCTTCA | |
| | ACGGCCTCGCGGACCTGGCGGGCTCTACGGCGGGCGACTACGGCGGCCTC | |
| | GTGGTCTTCTGCATCTTCTTTGGCATCTCCTACGGCATGGTGGGGGCCCTG | |
| | CAGTTCGAGGTGCTCATGGCCATCGTGGGCACCCACAAGTTCTCCAGTGC | |
| | CATTGGCCTGGTGCTGCTGATGGAGGCGGTGGCCGTGCTCGTCGGGCCCC | |
| | CTTCGGGAGGCAAACTCCTGGATGCGACCCACGTCTACATGTACGTGTTC | |
| | ATCCTGGCGGGGGCCGAGGTGCTCACCTCCTCCCTGATTTTGCTGCTGGGC | |
| | AACTTCTTCTGCATTAGGAAGAAGCCCAAAGAGCCACAGCCTGAGGTGGC | |
| | GGCCGCGGAGGAGGAGAAGCTCCACAAGCCTCCTGCAGACTCGGGGGTG | |
| | GACTTGCGGGAGGTGGAGCATTTCCTGAAGGCTGAGCCTGAGAAAAACG | |
| | GGGAGGTGGTTCACACCCCGGAAACAAGTGTCTGAGTGGCTGGGCGGGG | |
| | CCGGCAGGCACAGGGAGGAGGTACAGAAGCCGGCAACGCTTGCTATTTA | |
| | TTTTACAAACTGGACTGGCTCAGGCAGGGCCACGGCTGGGCTCCAGCTGC | |
| | CGGCCCAGCGGATCGTCGCCCGATCAGTGTTTTGAG | |
| C14ORF58 | GCGAGGCCCCAAGCTGGCCCGGGAGAGGACTCTGCGGGCGAAGTGGCTG | 210 |
| NM_017791.2 | CGCAAGGAGAGAACTTTTCCTGCACAAGGAACGCCTCGTGGGGAGACCC | |
| | AAGGCAGGAGCGGTCCGGAGCCGGCTGCGGCGTGTGCGGCCGGCCTTGG | |
| | GACAGCGATCGCCGCGGGTGGCAACAGAGAGCCCCAAGCAAAAGTGGGA | |
| | GCAGGAGCTTGGAGGTGAGCACAGGAAGCCCCACTTGAGGCTTTTACGCA | |
| | GCCTCTAGTCTCTGTTTCTTCTGGAATAGGCAAGTGTCCTTTCAACTCTAA | |
| | GAGACCAGCAGAGGCCACTGTCCCTTAAGACTGCCGGAGTCCTCACCACT | |
| | TCTCCAGGATTCCAGAGGAGACTGTGGCGATGGTGAATGAAGGTCCCAAC | |
| | CAGGAAGAGAGCGATGACACCCCTGTGCCGGAGTCCGCACTCCAAGCGG | |
| | ACCCCAGCGTCTCGGTCCATCCCAGCGTCTCGGTCCATCCCAGCGTCTCCA | |
| | TCAACCCCAGCGTCTCTGTCCACCCCAGCAGTTCGGCCCACCCCAGTGCCT | |
| | TAGCCCAACCCAGTGGCTTGGCTCACCCCAGTAGCTCGGGCCCTGAGGAC | |
| | CTCAGCGTGATCAAGGTGAGCAGGCGCCGTTGGGCCGTGGTCCTGGTGTT | |
| | TAGCTGCTACTCCATGTGCAACTCCTTTCAGTGGATCCAGTACGGCTCCAT | |
| | CAATAACATCTTCATGCACTTCTACGGTGTCAGTGCCTTTGCCATTGACTG | |
| | GCTGTCCATGTGCTACATGCTGACTTACATCCCTCTGCTCCTGCCAGTGGC | |
| | TTGGCTGCTGGAGAAGTTCGGCCTGCGCACCATTGCTCTCACTGGCTCGG | |
| | CTCTCAACTGCCTGGGGGCCTGGGTGAAGCTGGGCAGCCTGAAGCCGCAT | |
| | CTCTTTCCGGTCACCGTGGTGGGCCAGCTCATCTGCTCTGTGGCCCAGGTT | |
| | TTCATCCTGGGCATGCCCTCCCGCATCGCTTCCGTCTGGTTCGGGGCTAAT | |
| | GAGGTTTCAACAGCCTGCTCCGTGGCTGTCTTTGGCAATCAGCTTGGAATT | |
| | GCGATTGGGTTCTTGGTCCCTCCTGTTTTGGTACCCAACATTGAAGACCGG | |
| | GACGAGCTTGCCTACCACATCAGCATCATGTTCTATATAATAGGAGGTGT | |
| | GGCCACTCTCCTCCTCATCCTTGTCATCATTGTGTTCAAGGAGAAACCTAA | |
| | ATATCCCCCCAGCAGGGCCCAATCCCTGAGCTATGCCTTGACCTCTCCTGA | |
| | TGCCTCATACTTAGGTTCCATCGCCCGGCTCTTCAAAAATCTCAACTTTGT | |
| | GCTGCTTGTCATCACCTATGGTCTGAATGCTGGTGCTTTTTATGCCTTGTC | |
| | CACTCTTCTGAATCGCATGGTGATCTGGCACTACCCGGGGGAAGAAGTGA | |
| | ATGCTGGAAGAATTGGCCTGACGATCGTCATTGCAGGAATGCTTGGGGCT | |
| | GTGATCTCAGGAATCTGGCTGGATAGGTCCAAAACCTACAAAGAGACAA | |
| | CCCTGGTAGTCTATATCATGACACTGGTGGGCATGGTGGTGTACACGTTT | |
| | ACCTTGAACCTGGGACACCTGTGGGTAGTGTTCATCACTGCTGGCACAAT | |
| | GGGCTTCTTTATGACTGGCTATCTCCCACTGGGATTTGAGTTTGCTGTGGA | |
| | GCTCACGTACCCAGAATCAGAAGGCATCTCCTCCGGCCTCCTCAACATAT | |
| | CTGCACAGGTATTTGGGATCATCTTTACCATCTCCCAGGGCCAGATTATTG | |
| | ACAACTATGGAACCAAGCCTGGGAACATCTTCCTGTGTGTGTTCCTTACTC | |
| | TTGGAGCAGCCCTCACTGCATTCATTAAGGCAGATCTCCGGAGACAGAAA | |
| | GCAAACAAAGAAACTCTTGAGAACAAACTCCAAGAGGAGGAGGAGGAG | |
| | AGCAACACCAGCAAAGTGCCCACTGCTGTGTCAGAGGATCATCTCTGAGA | |
| | GGAAGGTGGTGACAACTCAGGGAACACGAACACCCCACCTTTTCCTTCAG | |
| | CACAGCTCTCACCGCCAGCACAAAGGGCTTCGCTAGAGATGTTTTTGGAG | |
| | GGAATCAGTGGGACTATTTGTGGCATGGATGGCCTATTCCTCCTAGAACC | |
| | CACGTAAGAGCTTGGATGATTTAGTTGGAGAAAATTGCACCTATCACCAA | |
| | ATGCAAATTTGATTCCCACCTCCACCCCCTTTTAGGTTATGGGAGTTGGTG | |
| | TTGGGACAGGGTGGCAGAGAATATTGGAGTCAATCCTAGCTTGGTCTCTT | |
| | GCCTTCCCTCTTTTCCTCCATCCATCGTGGACAATGCCTGCAAAATTTTCA | |
| | CAGGAAGAAAGCCTATTCAGGATATTAACTTGAAATTTCCAGTGTCCTAA | |
| | GAGCCTCTCATGAAGCCCAGTTCTAATAAGTGGCAAGCTGCTCTGCCGGG | |
| | GTCATCTCCTGGGTCATCGGACTGATTGCTCAAGTTCTGCAGGAGAGGAA | |
| | GCACCATTAGAACAACTCCATCAGAACAGCTCCACCGGGACTTGTGGGCC | |
| | TAAATTTTCCTGGCCTAACGGGTCTGTCTCCAAACCCTCTTTCCTAAGAGC | |
| | TGAGCAAACCAACCATAATAAACTTGACAAAAGACTTTGTTGTGGCCATG | |
| | ACAGAGATACCGACTCAGGAGGGCTACCTACCTAGGTGTGATCATGCTGG | |
| | GGGCTACCTTCTGAGTATATTTGTGAAAGCACATATTTGGGAACTCTGGT | |
| | AGCTTGAGTTGGGAATGGGAAGGTTCTTTTTTACAGAAGTACTTCCCCAG | |
| | GGACTTCTGTGTGTCACAGTCACCTCTGATGCCTTTATCTTGATGTTGCAT | |
| | TGGGAATCTCAGCCATCAGCCCAAGTGCTTGTTTTATTCCAAGGCAGGGT | |
| | AATCCCCGTCAACTTACTCTAACCTTTGCTGAAAACTAATCTTGATTCATT | |
| | CTACTCTGAAAATCCAAAGGTGCTTCTGAGAGATAAGAGGGAAGGGGTA | |
| | GAAGGAAAGGTGCCCCTTGAAATGGGAATTGAGCCTGTTAGAATTAAAA | |
| | GCTTATCTCACCTCTGCTGGGGACAGTATTTGCACCACCAACCCCTCTCCT | |
| | CACCTGCTTTGAGCGATAATCTTTATCAGATATTCTAAACTTAAAGGGATT | |
| | CCCTTTAAACCAACTCAAGCTGATCTTTCCTATCTAGCCTGCTGTTTGGCT | |
| | GTACTCATGGGCTTTGGTAATATCTCCTAAAAATGAGGTTTTGGTAATTTT | |
| | TCCTATGCATTGGGCAACTGTGATCGTGACCACTGTGCTGTCTTGCTCCAG | |
| | CCACTGCCCTGGCCTCAGCATATCAGGGCAGCCTGTGCTGGCTGCAATAC | |
| | TGTGGTGCTTGGGCCACTGCCTGAGAGGAGCCAGGTTTGTGTGTGTCTGC | |
| | ATGTGTGTGTGTGTGTGTTTGTACAGATTCAAGCAATGGATGCAAGGAAC | |
| | ATGCTGTATGTAATAGAAGAAAGAAGTCCACGTTTTCGGCAGAAGTAGTG | |
| | AGTCAGTGTGGAAGAGAGGTGAGGGTGTGCTTTACTTTTTGATAAAGAGA | |
| | AAGATGTTTACTCATAAACCCTTCAAAAGGTATTAACAAAATGTTTACCA | |
| | AACCTATTGCTTTATTTTAAAAACATAATTTGTGTTTTCTATTTGTAAGATC | |
| | TGACATTTCGAGGCAATAAAAACTTCTCAGAAAGTAAAAAAAAAAAAAA | |
| | A | |

Preferably, the expression of each of the 13-gene set is determined to provide the VEGF-signature score. An average expression value across the genes can be determined, i.e., by determining a log₂ expression ratio. The sample may be assigned or classified into a high expression group, an intermediate expression group, and a low expression group based on the 13-gene average log₂ expression ratio using cutoff values (i.e., −0.63/0.08) identified using X-tile and relapse-free survival, as described in Camp et al., Clin. Cancer Res. 10(21):7252-7259. The methods for determining the VEGF-signature score from a biological sample are as described in Hu et al., BMC Medicine 7:9 (2009) and supplemental online material.

The methods of the present invention may further include measuring the expression of DNA repair genes, such as RAD17, RAD50, and tumor suppressor RB1. Select nucleic acid sequences for these additional genes are shown in Table 8 below.

TABLE 8

| GENE NAME | | |
|---|---|---|
| GENBANK | | SEQ |
| ACCESSION | | ID |
| NUMBER | SEQUENCE | NO: |
| RAD17 | ACCTGTATACCTTTTGAAACGTCACAGTCTCTAATCGTGAACGATTT | 211 |
| AF076838.1 | GGGGCGGAGGGCTGAACAATGTGTTTTCTAGTGTGTCGAGGTGTTTA | |
| | TAGGCTATGTGTGCCTCCAAACTGTAAAGTAGTCCAGTATACTTTCC | |
| | AATGTATAAGTTTGTAGACCTTAAACTTTTCTTCTGGCTAACTTAAAA | |
| | TCGTTGAATTCACTAGTTTGCATAAACATTTAAGAATTTGAAAACAC | |
| | GGTTGAAAAACAGTGTTACCAAGAAATTTTGTAATAACATGTTCAAA | |
| | TGAAGACAAAAATTTTACAGTTTAAGACTTAAATTCTTCGTCCACAG | |
| | CAAGTGAATTCATGGTATTTTACTTTTTTGGGAAATACTGGAAATGA | |
| | AGACCTGCAACTGTAATTTGAAATAAGGAAAACTTTAATTTTCAGTA | |
| | TAAAAATTGCTCAAATAGAATTGCCTGATTTTAATGACAAAAGTATA | |
| | TGGGAGTCCACATTTATGTAAGAAATGAAACTATAAAATGTATAAAT | |
| | AATTTGCAAATCAGAATTGCTGTCGAAAGTTTTACTATAATGAAAGA | |
| | TATTTTCATACTCTCAAAAATATAGAGGAAAGGGGCCAAGATTATAG | |
| | TACCAGTCACAATCTTTTGATGAGGACGAAATGAATCAGGTAACAG | |
| | ACTGGGTTGACCCATCATTTGATGATTTTCTAGAGTGTAGTGGCGTCT | |
| | CTACTATTACTGCCACATCATTAGGTGTGAATAACTCAAGTCATAGA | |
| | AGAAAAAATGGGCCTTCTACATTAGAAAGCAGCAGATTTCCAGCGA | |
| | GAAAAAGAGGAAATCTATCTTCCTTAGAACAGATTTATGGTTTAGAA | |
| | AATTCAAAAGAATATCTGTCTGAAAATGAACCATGGGTGGATAAAT | |
| | ATAAACCAGAAACTCAGCATGAACTTGCTGTGCATAAAAAGAAAAT | |
| | TGAAGAAGTCGAAACCTGGTTAAAAGCTCAAGTTTTAGAAAGGCAA | |
| | CCAAAACAGGGTGGATCTATTTTATTAATAACAGGTCCTCCTGGATG | |
| | TGGAAAGACAACGACCTTAAAAATACTATCAAAGGAGCATGGTATT | |
| | CAAGTACAAGAGTGGATTAATCCAGTTTTACCAGACTTCCAAAAAG | |
| | ATGATTTCAAGGGGATGTTTAATACTGAATCAAGCTTCCATATGTTT | |
| | CCCTATCAGTCTCAGATAGCAGTTTTCAAAGAGTTTCTACTAAGAGC | |
| | GACAAAGTATAACAAGTTACAAATGCTTGGAGATGATCTGAGAACT | |
| | GATAAGAAGATAATTCTGGTTGAAGATTTACCTAACCAGTTTTATCG | |
| | GGATTCTCATACTTTACATGAAGTTCTAAGGAAGTATGTGAGGATTG | |
| | GTCGATGTCCTCTTATATTTATAATCTCGGACAGTCTCAGTGGAGAT | |
| | AATAATCAAAGGTTATTGTTTCCCAAAGAAATTCAGGAAGAGTGTTC | |
| | TATCTCAAATATTAGTTTCAACCCTGTGGCACCAACAATTATGATGA | |
| | AATTTCTTAATCGAATAGTGACTATAGAAGCTAACAAGAATGGAGG | |
| | AAAAATTACTGTCCCTGACAAAACTTCTCTAGAGTTGCTCTGTCAGG | |
| | GATGTTCTGGTGATATCAGAAGTGCAATAAACAGCCTCCAGTTTTCT | |
| | TCTTCAAAAGGAGAAAACAACTTACGGCCAAGGAAAAAAGGAATGT | |
| | CTTTAAAATCAGATGCTGTGCTGTCAAAATCAAAACGAAGAAAAAA | |
| | ACCTGATAGGGTTTTTGAAAATCAAGAGGTCCAAGCTATTGGTGGCA | |
| | AAGATGTTTCTCTGTTTCTCTTCAGAGCTTTGGGGAAAATTCTATATT | |
| | GTAAAAGAGCATCTTTAACAGAATTAGACTCACCTCGGTTGCCCTCT | |
| | CATTTATCAGAATATGAACGGGATACATTACTTGTTGAACCTGAGGA | |
| | GGTAGTAGAAATGTCACACATGCCTGGAGACTTATTTAATTTATATC | |
| | TTCACCAAAACTACATAGATTTCTTCATGGAAATTGATGATATTGTG | |
| | AGAGCCAGTGAATTTCTGAGTTTTGCAGATATCCTCAGTGGTGACTG | |
| | GAATACACGCTCTTTACTCAGGGAATATAGCACATCTATAGCTACGA | |
| | GAGGTGTGATGCATTCCAACAAAGCCCGAGGATATGCTCATTGCCA | |
| | AGGAGGAGGATCAAGTTTTCGACCCTTGCACAAACCTCAGTGGTTTC | |
| | TAATAAATAAAAAGTATCGGGAAAATTGCCTGGCAGCAAAAGCACT | |
| | TTTTCCTGACTTCTGCCTACCAGCTTTATGCCTCCAAACTCAGCTATT | |
| | GCCATACCTTGCTCTACTAACCATTCCAATGAGAAATCAAGCTCAGA | |
| | TTTCTTTTATCCAAGATATTGGAAGGCTCCCTCTGAAGCGACACTTTG | |
| | GAAGATTGAAAATGGAAGCCCTGACTGACAGGGAACATGGAATGAT | |
| | AGACCCTGACAGCGGAGATGAAGCCCAGCTTAATGGAGGACATTCT | |
| | GCAGAGGAATCTCTGGGTGAACCCACTCAAGCCACTGTGCCGGAAA | |
| | CCTGGTCTCTTCCTTTGAGTCAGAATAGTGCCAGTGAACTGCCTGCT | |
| | AGCCAGCCCCAGCCCTTTTCAGCCCAAGGAGACATGGAAGAAAACA | |
| | TAATAATAGAAGACTACGAGAGTGATGGGACATAGAAGCCAGCCTG | |
| | CTAATCAGATTGCTACTTCACAGCTTCATTTTTGTTTCATTCAGTGGT | |
| | ACTTCAGCAGAGTTAATATGCTTTTCTGATGAATTACACAACAGTTT | |
| | GTTAATTCTTCATTCTTGTAGTATTTCATCACAAGAAACCTACTCTTC | |
| | TGTCATCTTGAAGTAAATAGAAGATCAAGCCTTCAAATCTCTTAATT | |
| | TTTTCGGTATTTATTAAATCTGTGAGTGGTTTAAGGAGCGGTCAGTGT | |
| | GTATAAAGTGTGTTTGAACATTATGCCAAATATCAAGATGTGAAGGA | |
| | CTAATTCAGGATGCAAAAACGTTATTGGGGGGTTGTAAATATCAACT | |
| | ATTCAACAGTTTAGGATGCAATTACGAGTGTAAACTGTGTGCCTTAT | |
| | TTACACTTTATTGTCTCCCGCTTCTCAGATAGTTTTGATGTGTTGTAC | |
| | AGTGGAATATCTTAGATACTTTTTGGAAAGTATTTACATAAGTTATA | |
| | TCACAATTAAAATGTTGAATTTAA | |
| RAD50 | TTTCCCGGCGTGCCCCAGGAGAGCGGCGTGGACGCGTGCGGGCCTA | 212 |
| NM_005732.3 | GAGGCCCACGTGATCCGCAGGGCGGCCGAGGCAGGAAGCTGTGAGT | |
| | GCGCGGTTGCGGGGTCGCATTGTGGCTACGGCTTTGCGTCCCCGGCG | |
| | GGCAGCCCCAGGCTGGTCCCCGCCTCCGCTCTCCCCACCGGCGGGGA | |
| | AAGCAGCTGGTGTGGGAGGAAAGGCTCCATCCCCCGCCCCCTCTCTC | |
| | CCGCTGTTGGCTGGCAGGATCTTTTGGCAGTCCTGTGGCCTCGCTCC | |
| | CCGCCCGGATCCTCCTGACCCTGAGATTCGCGGGTCTCACGTCCCGT | |
| | GCACGCCTTGCTTCGGCCTCAGTTAAGCCTTTGTGGACTCCAGGTCC | |
| | CTGGTGAGATTAGAAACGTTTGCAAACATGTCCCGGATCGAAAAGA | |
| | TGAGCATTCTGGGCGTGCGGAGTTTTGGAATAGAGGACAAAGATAA | |
| | GCAAATTATCACTTTCTTCAGCCCCCTTACAATTTTGGTTGGACCCAA | |
| | TGGGGCGGGAAAGACGACCATCATTGAATGTCTAAAATATATTTGTA | |
| | CTGGAGATTTCCCTCCTGGAACCAAAGGAAATACATTTGTACACGAT | |
| | CCCAAGGTTGCTCAAGAAACAGATGTGAGAGCCCAGATTCGTCTGC | |
| | AATTTCGTGATGTCAATGGAGAACTTATAGCTGTGCAAAGATCTATG | |
| | GTGTGTACTCAGAAAAGCAAAAAGACAGAATTTAAAACTCTGGAAG | |
| | GAGTCATTACTAGAACAAAGCATGGTGAAAAGGTCAGTCTGAGCTC | |
| | TAAGTGTGCAGAAATTGACCGAGAAATGATCAGTTCTCTTGGGGTTT | |
| | CCAAGGCTGTGCTAAATAATGTCATTTTCTGTCATCAAGAAGATTCT | |
| | AATTGGCCTTTAAGTGAAGGAAAGGCTTTGAAGCAAAAGTTTGATG | |
| | AGATTTTTTCAGCAACAAGATACATTAAAGCCTTAGAAACACTTCGG | |
| | CAGGTACGTCAGACACAAGGTCAGAAAGTAAAAGAATATCAAATGG | |
| | AACTAAAATATCTGAAGCAATATAAGGAAAAAGCTTGTGAGATTCG | |
| | TGATCAGATTACAAGTAAGGAAGCCCAGTTAACATCTTCAAAGGAA | |
| | ATTGTCAAATCCTATGAGAATGAACTTGATCCATTGAAGAATCGTCT | |
| | AAAAGAAATTGAACATAATCTCTCTAAAATAATGAAACTTGACAAT | |
| | GAAATTAAAGCCTTGGATAGCCGAAAGAAGCAAATGGAGAAAGATA | |
| | ATAGTGAACTGGAAGAGAAAATGGAAAAGGTTTTTCAAGGGACTGA | |
| | TGAGCAACTAAATGACTTATATCACAATCACCAGAGAACAGTAAGG | |
| | GAGAAAGAAAGGAAATTGGTAGACTGTCATCGTGAACTGGAAAAAC | |
| | TAAATAAAGAATCTAGGCTTCTCAATCAGGAAAAATCAGAACTGCTT | |
| | GTTGAACAGGGTCGTCTACAGCTGCAAGCAGATCGCCATCAAGAAC | |
| | ATATCCGAGCTAGAGATTCATTAATTCAGTCTTTGGCAACACAGCTA | |
| | GAATTGGATGGCTTTGAGCGTGGACCATTCAGTGAAAGACAGATTA | |
| | AAAATTTTCACAAACTTGTGAGAGAGAGACAAGAAGGGGAAGCAAA | |
| | AACTGCCAACCAACTGATGAATGACTTTGCAGAAAAAGAGACTCTG | |
| | AAACAAAAACAGATAGATGAGATAAGAGATAAGAAAACTGGACTG | |
| | GGAAGAATAATTGAGTTAAAATCAGAAATCCTAAGTAAGAAGCAGA | |
| | ATGAGCTGAAAAATGTGAAGTATGAATTACAGCAGTTGGAAGGATC | |
| | TTCAGACAGGATTCTTGAACTGGACCAGGAGCTCATAAAAGCTGAA | |
| | CGTGAGTTAAGCAAGGCTGAGAAAAACAGCAATGTAGAAACCTTAA | |
| | AAATGGAAGTAATAAGTCTCCAAAATGAAAAAGCAGACTTAGACAG | |
| | GACCCTGCGTAAACTTGACCAGGAGATGGAGCAGTTAAACCATCAT | |
| | ACAACAACACGTACCCAAATGGAGATGCTGACCAAAGACAAAGCTG | |
| | ACAAAGATGAACAAATCAGAAAAATAAAATCTAGGCACAGTGATGA | |
| | ATTAACCTCACTGTTGGGATATTTTCCCAACAAAAAACAGCTTGAAG | |
| | ACTGGCTACATAGTAAATCAAAAGAAATTAATCAGACCAGGGACAG | |
| | ACTTGCCAAATTGAACAAGGAACTAGCTTCATCTGAGCAGAATAAA | |
| | AATCATATAAATAATGAACTAAAAAGAAAGGAAGAGCAGTTGTCCA | |
| | GTTACGAAGACAAGCTGTTTGATGTTTGTGGTAGCCAGGATTTTGAA | |
| | AGTGATTTAGACAGGCTTAAAGAGGAAATTGAAAAATCATCAAAAC | |
| | AGCGAGCCATGCTGGCTGGAGCCACAGCAGTTTACTCCCAGTTCATT | |
| | ACTCAGCTAACAGACGAAAACCAGTCATGTTGCCCCGTTTGTCAGAG | |
| | AGTTTTTCAGACAGAGGCTGAGTTACAAGAAGTCATCAGTGATTTGC | |
| | AGTCTAAACTGCGACTTGCTCCAGATAAACTCAAGTCAACAGAATCA | |
| | GAGCTAAAAAAAAAGGAAAAGCGGCGTGATGAAATGCTGGGACTTG | |
| | TGCCCATGAGGCAAAGCATAATTGATTTGAAGGAGAAGGAAATACC | |
| | AGAATTAAGAAACAAACTGCAGAATGTCAATAGAGACATACAGCGC | |
| | CTAAAGAACGACATAGAAGAACAAGAAACACTCTTGGGTACAATAA | |
| | TGCCTGAAGAAGAAAGTGCCAAAGTATGCCTGACAGATGTTACAAT | |
| | TATGGAGAGGTTCCAGATGGAACTTAAAGATGTTGAAAGAAAAATT | |
| | GCACAACAAGCAGCTAAGCTACAAGGAATAGACTTAGATCGAACTG | |
| | TCCAACAAGTCAACCAGGAGAAACAAGAGAAACAGCACAAGTTAG | |
| | ACACAGTTTCTAGTAAGATTGAATTGAATCGTAAGCTTATACAGGAC | |
| | CAGCAGGAACAGATTCAACATCTAAAAAGTACAACAAATGAGCTAA | |
| | AATCTGAGAAACTTCAGATATCCACTAATTTGCAACGTCGTCAGCAA | |
| | CTGGAGGAGCAGACTGTGGAATTATCCACTGAAGTTCAGTCTTTGTA | |
| | CAGAGAGATAAAGGATGCTAAAGAGCAGGTAAGCCCTTTGGAAACA | |
| | ACATTGGAAAAGTTCCAGCAAGAAAAAGAAGAATTAATCAACAAAA | |
| | AAAATACAAGCAACAAAATAGCACAGGATAAACTGAATGATATTAA | |
| | AGAGAAGGTTAAAAATATTCATGGCTATATGAAAGACATTGAGAAT | |
| | TATATTCAAGATGGGAAAGACGACTATAAGAAGCAAAAAGAAACTG | |
| | AACTTAATAAAGTAATAGCTCAACTAAGTGAATGCGAGAAACACAA | |
| | AGAAAAGATAAATGAAGATATGAGACTCATGAGACAAGATATTGAT | |
| | ACACAGAAGATACAAGAAAGGTGGCTACAAGATAACCTTACTTTAA | |
| | GAAAAAGAAATGAGGAACTAAAAGAAGTTGAAGAAGAAAGAAAAC | |
| | AACATTTGAAGGAAATGGGTCAAATGCAGGTTTTGCAAATGAAAAG | |
| | TGAACATCAGAAGTTGGAAGAGAACATAGACAATATAAAAAGAAAT | |
| | CATAATTTGGCATTAGGGCGACAGAAAGGTTATGAAGAAGAAATTA | |
| | TTCATTTTAAGAAAGAACTTCGAGAACCACAATTTCGGGATGCTGAG | |
| | GAAAAGTATAGAGAAATGATGATTGTTATGAGGACAACAGAACTTG | |
| | TGAACAAGGATCTGGATATTTATTATAAGACTCTTGACCAAGCAATA | |
| | ATGAAATTTCACAGTATGAAAATGGAAGAAATCAATAAAATTATAC | |
| | GTGACCTGTGGCGAAGTACCTATCGTGGACAAGATATTGAATACATA | |
| | GAAATACGGTCTGATGCCGATGAAAATGTATCAGCTTCTGATAAAAG | |
| | GCGGAATTATAACTACCGAGTGGTGATGCTGAAGGGAGACACAGCC | |
| | TTGGATATGCGAGGACGATGCAGTGCTGGACAAAAGGTATTAGCCT | |
| | CACTCATCATTCGCCTGGCCCTGGCTGAAACGTTCTGCCTCAACTGT | |
| | GGCATCATTGCCTTGGATGAGCCAACAACAAATCTTGACCGAGAAA | |
| | ACATTGAATCTCTTGCACATGCTCTGGTTGAGATAATAAAAAGTCGC | |
| | TCACAGCAGCGTAACTTCCAGCTTCTGGTAATCACTCATGATGAAGA | |
| | TTTTGTGGAGCTTTTAGGACGTTCTGAATATGTGGAGAAATTCTACA | |
| | GGATTAAAAAGAACATCGATCAGTGCTCAGAGATTGTGAAATGCAG | |
| | TGTTAGCTCCCTGGGATTCAATGTTCATTAAAAATATCCAAGATTTA | |
| | AATGCCATAGAAATGTAGGTCCTCAGAAAGTGTATAATAAGAAACT | |
| | TATTTCTCATATCAACTTAGTCAATAAGAAAATATATTCTTTCAAAG | |
| | GAACATTGTGTCTAGGATTTTGGATGTTGAGAGGTTCTAAAATCATG | |
| | AAACTTGTTTCACTGAAAATTGGACAGATTGCCTGTTTCTGATTTGCT | |
| | GCTCTTCATCCCATTCCAGGCAGCCTCTGTCAGGCCTTCAGGGTTCA | |
| | GCAGTACAGCCGAGACTCGACTCTGTGCCTCCCTCCCCAGTGCAAAT | |
| | GCATGCTTCTTCTCAAAGCACTGTTGAGAAGGAGATAATTACTGCCT | |
| | TGAAAATTTATGGTTTTGGTATTTTTTTAAATCATAGTTAAATGTTAC | |
| | CTCTGAATTTACTTCCTTGCATGTGGTTTGAAAAACTGAGTATTAATA | |
| | TCTGAGGATGACCAGAAATGGTGAGATGTATGTTTGGCTCTGCTTTT | |
| | AACTTTATAAATCCAGTGACCTCTCTCTCTGGGACTTGGTTTCCCCAA | |
| | CTAAAATTTGAAGTAGTTGAATGGGGTCTCAAAGTTTGACAGGAACC | |
| | TTAAGTAATCATCTAAGTCAGTACCCACCACCTTCTTCTCCTACATAT | |
| | CCCTTCCAGATGGTCATCCAGACTCAGAGCTCTCTCTACAGAGAGGA | |
| | AATTCTCCACTGTGCACACCCACCTTTGGAAAGCTCTGACCACTTGA | |
| | GGCCTGATCTGCCCATCGTGAAGAAGCCTGTAACACTCCTCTGCGTC | |
| | TATCCTGTGTAGCATACTGGCTTCACCATCAATCCTGATTCCTCTCTA | |
| | AGTGGGCATTGCCATGTGGAAGGCAAGCCAGGCTCACTCACAGAGT | |
| | CAAGGCCTGCTCCCTGTAGGGTCCAACCAGACCTGGAAGAACAGGC | |
| | CTCTCCATTTGCTCTTCAGATGCCACTTCTAAGAAAAGCCTAATCAC | |
| | AGTTTTTCCTGGAATTGCCAGCTGACATCTTGAATCCTTCCATTCCAC | |
| | ACAGAATGCAACCAAGTCACACGCTTTTGAATTATGCTTTGTAGAGT | |
| | TTTGTCATTCAGAGTCAGCCAGGACCATACCGGGTCTTGATTCAGTC | |
| | ACATGGCATGGTTTTGTGCCATCTGTAGCTATAATGAGCATGTTTGC | |
| | CTAGACAGCTTTTCTCAACTGGGTCCAGAAGAGAATTAAGCCCTAAG | |
| | GTCCTAAGGCATCTATCTGTGCTAGGTTAAATGGTTGGCCCCCAAAG | |
| | ATAGACAGGTCCTGATTTCTAGAACCCGTGACTGTTACTTTATACAG | |
| | CAAAGGAAACTTTGCAGATGTGATTAAAGCTAAGGACCTTAAGACA | |
| | GAGTATCCTGGGGGTGGTGGTGGGGTGGGGGGGGGTCCTAAATGTA | |
| | ATCACGAGTAAGATTAAGAGCAAATCAATTCTAGTCATATATTAAAC | |
| | ATCCACAATAACCAAGATATTTTTATCCCAAGAATGCAAGATTTCAG | |
| | AAAATGAAAAATCTGTTGATAAATCCATCACTATAATAAAACCGAA | |
| | GGTGAAAAAAATTCTGAAAAAATTCTAGCAGCTATATTTGATAAAAT | |
| | TCAACATCTCCTAGCTTTAGCAAACTCACAGTTTTGCAAATAATATTT | |
| | TCTTAATGTTATCTGTTGCTAAATCAAAATTAAACAGTCATCTTAACT | |
| | GCAAAATAAAACATTTCTCAGTAAATATTAAAGCCAGTTACCTTCTA | |
| | TCAACATGTTAATGAAAGTGCTAGTTGTTGCAGCAAAGAATAACAA | |
| | AGGCAATACACGATCAATATAGGCAGTGAAACAAAAGTATCATTTG | |
| | CAAGTTAAAACAGACTTCCCAATTTTAAATCTGGTTTCCCCCTGAAT | |
| | ATGTGGCATCCTTGGCAGCACTTCTGAGAGTGGCTGCTTTCATTCCA | |
| | AGAAGCCCATGGGTTTGGAGGTGGGATAGGTGCCTTTCTGGCTTCTC | |
| | ATTGCTGCTTCTAGATCAGTCTCCAAATATCCCCCTTCCCCACATTGG | |
| | AATGAATAGCCATCACAGCATGGATGGAGGTTAGAATGAGCCAGAC | |
| | TGCCTGGGCTCAAATCCTAGCACACCACTCACTAGCTGGGGACCTTG | |
| | AGCAAGTTATTTGTCCTGTTTTCTGTTTCCTTATATGTAAAAGTGGGT | |
| | AAAATGGTACATATTTTGTAGGGTTGTTATGAAGATTGAATGACATT | |
| | ATTTACAAACTGCTTAGAACTGCTTGCCACCTACTAAATACTGTGTA | |
| | AGTGTTCAAGAAAAAGCTGTCTTCATTTCA | |
| RB1 | GCTCAGTTGCCGGGCGGGGGAGGGCGCGTCCGGTTTTTCTCAGGGG | 213 |
| NM_000321.2 | ACGTTGAAATTATTTTTGTAACGGGAGTCGGGAGAGGACGGGGCGT | |
| | GCCCCGACGTGCGCGCGCGTCGTCCTCCCCGGCGCTCCTCCACAGCT | |
| | CGCTGGCTCCCGCCGCGGAAAGGCGTCATGCCGCCCAAAACCCCCC | |
| | GAAAAACGGCCGCCACCGCCGCCGCTGCCGCCGCGGAACCCCCGGC | |
| | ACCGCCGCCGCCGCCCCCTCCTGAGGAGGACCCAGAGCAGGACAGC | |
| | GGCCCGGAGGACCTGCCTCTCGTCAGGCTTGAGTTTGAAGAAACAG | |
| | AAGAACCTGATTTTACTGCATTATGTCAGAAATTAAAGATACCAGAT | |
| | CATGTCAGAGAGAGAGCTTGGTTAACTTGGGAGAAAGTTTCATCTGT | |
| | GGATGGAGTATTGGGAGGTTATATTCAAAAGAAAAAGGAACTGTGG | |
| | GGAATCTGTATCTTTATTGCAGCAGTTGACCTAGATGAGATGTCGTT | |
| | CACTTTTACTGAGCTACAGAAAAACATAGAAATCAGTGTCCATAAAT | |
| | TCTTTAACTTACTAAAAGAAATTGATACCAGTACCAAAGTTGATAAT | |
| | GCTATGTCAAGACTGTTGAAGAAGTATGATGTATTGTTTGCACTCTT | |
| | CAGCAAATTGGAAAGGACATGTGAACTTATATATTTGACACAACCC | |
| | AGCAGTTCGATATCTACTGAAATAAATTCTGCATTGGTGCTAAAAGT | |
| | TTCTTGGATCACATTTTTATTAGCTAAAGGGGAAGTATTACAAATGG | |
| | AAGATGATCTGGTGATTTCATTTCAGTTAATGCTATGTGTCCTTGACT | |
| | ATTTTATTAAACTCTCACCTCCCATGTTGCTCAAAGAACCATATAAA | |
| | ACAGCTGTTATACCCATTAATGGTTCACCTCGAACACCCAGGCGAGG | |
| | TCAGAACAGGAGTGCACGGATAGCAAAACAACTAGAAAATGATACA | |
| | AGAATTATTGAAGTTCTCTGTAAAGAACATGAATGTAATATAGATGA | |
| | GGTGAAAAATGTTTATTTCAAAAATTTTATACCTTTTATGAATTCTCT | |
| | TGGACTTGTAACATCTAATGGACTTCCAGAGGTTGAAAATCTTTCTA | |
| | AACGATACGAAGAAATTTATCTTAAAAATAAAGATCTAGATGCAAG | |
| | ATTATTTTTGGATCATGATAAAACTCTTCAGACTGATTCTATAGACA | |
| | GTTTTGAAACACAGAGAACACCACGAAAAAGTAACCTTGATGAAGA | |
| | GGTGAATGTAATTCCTCCACACACTCCAGTTAGGACTGTTATGAACA | |
| | CTATCCAACAATTAATGATGATTTTAAATTCAGCAAGTGATCAACCT | |
| | TCAGAAAATCTGATTTCCTATTTTAACAACTGCACAGTGAATCCAAA | |
| | AGAAAGTATACTGAAAAGAGTGAAGGATATAGGATACATCTTTAAA | |
| | GAGAAATTTGCTAAAGCTGTGGGACAGGGTTGTGTCGAAATTGGATC | |
| | ACAGCGATACAAACTTGGAGTTCGCTTGTATTACCGAGTAATGGAAT | |
| | CCATGCTTAAATCAGAAGAAGAACGATTATCCATTCAAAATTTTAGC | |
| | AAACTTCTGAATGACAACATTTTTCATATGTCTTTATTGGCGTGCGCT | |
| | CTTGAGGTTGTAATGGCCACATATAGCAGAAGTACATCTCAGAATCT | |
| | TGATTCTGGAACAGATTTGTCTTTCCCATGGATTCTGAATGTGCTTAA | |
| | TTTAAAAGCCTTTGATTTTTACAAAGTGATCGAAAGTTTTATCAAAG | |
| | CAGAAGGCAACTTGACAAGAGAAATGATAAAACATTTAGAACGATG | |
| | TGAACATCGAATCATGGAATCCCTTGCATGGCTCTCAGATTCACCTT | |
| | TATTTGATCTTATTAAACAATCAAAGGACCGAGAAGGACCAACTGAT | |
| | CACCTTGAATCTGCTTGTCCTCTTAATCTTCCTCTCCAGAATAATCAC | |
| | ACTGCAGCAGATATGTATCTTTCTCCTGTAAGATCTCCAAAGAAAAA | |
| | AGGTTCAACTACGCGTGTAAATTCTACTGCAAATGCAGAGACACAA | |
| | GCAACCTCAGCCTTCCAGACCCAGAAGCCATTGAAATCTACCTCTCT | |
| | TTCACTGTTTTATAAAAAAGTGTATCGGCTAGCCTATCTCCGGCTAA | |
| | ATACACTTTGTGAACGCCTTCTGTCTGAGCACCCAGAATTAGAACAT | |
| | ATCATCTGGACCCTTTTCCAGCACACCCTGCAGAATGAGTATGAACT | |
| | CATGAGAGACAGGCATTTGGACCAAATTATGATGTGTTCCATGTATG | |
| | GCATATGCAAAGTGAAGAATATAGACCTTAAATTCAAAATCATTGTA | |
| | ACAGCATACAAGGATCTTCCTCATGCTGTTCAGGAGACATTCAAACG | |
| | TGTTTTGATCAAAGAAGAGGAGTATGATTCTATTATAGTATTCTATA | |
| | ACTCGGTCTTCATGCAGAGACTGAAAACAAATATTTTGCAGTATGCT | |
| | TCCACCAGGCCCCCTACCTTGTCACCAATACCTCACATTCCTCGAAG | |
| | CCCTTACAAGTTTCCTAGTTCACCCTTACGGATTCCTGGAGGGAACA | |
| | TCTATATTTCACCCCTGAAGAGTCCATATAAAATTTCAGAAGGTCTG | |
| | CCAACACCAACAAAAATGACTCCAAGATCAAGAATCTTAGTATCAA | |
| | TTGGTGAATCATTCGGGACTTCTGAGAAGTTCCAGAAAATAAATCAG | |
| | ATGGTATGTAACAGCGACCGTGTGCTCAAAAGAAGTGCTGAAGGAA | |
| | GCAACCCTCCTAAACCACTGAAAAAACTACGCTTTGATATTGAAGGA | |
| | TCAGATGAAGCAGATGGAAGTAAACATCTCCCAGGAGAGTCCAAAT | |
| | TTCAGCAGAAACTGGCAGAAATGACTTCTACTCGAACACGAATGCA | |
| | AAAGCAGAAAATGAATGATAGCATGGATACCTCAAACAAGGAAGA | |
| | GAAATGAGGATCTCAGGACCTTGGTGGACACTGTGTACACCTCTGGA | |
| | TTCATTGTCTCTCACAGATGTGACTGTATAACTTTCCCAGGTTCTGTT | |
| | TATGGCCACATTTAATATCTTCAGCTCTTTTTGTGGATATAAAATGTG | |
| | CAGATGCAATTGTTTGGGTGATTCCTAAGCCACTTGAAATGTTAGTC | |
| | ATTGTTATTTATACAAGATTGAAAATCTTGTGTAAATCCTGCCATTTA | |
| | AAAAGTTGTAGCAGATTGTTTCCTCTTCCAAAGTAAAATTGCTGTGC | |
| | TTTATGGATAGTAAGAATGGCCCTAGAGTGGGAGTCCTGATAACCCA | |
| | GGCCTGTCTGACTACTTTGCCTTCTTTTGTAGCATATAGGTGATGTTT | |
| | GCTCTTGTTTTTATTAATTTATATGTATATTTTTTTAATTTAACATGAA | |
| | CACCCTTAGAAAATGTGTCCTATCTATCTTCCAAATGCAATTTGATTG | |
| | ACTGCCCATTCACCAAAATTATCCTGAACTCTTCTGCAAAAATGGAT | |
| | ATTATTAGAAATTAGAAAAAAATTACTAATTTTACACATTAGATTTT | |
| | ATTTTACTATTGGAATCTGATATACTGTGTGCTTGTTTTATAAAATTT | |
| | TGCTTTTAATTAAATAAAAGCTGGAAGCAAAGTATAACCATATGATA | |
| | CTATCATACTACTGAAACAGATTTCATACCTCAGAATGTAAAAGAAC | |
| | TTACTGATTATTTTCTTCATCCAACTTATGTTTTTAAATGAGGATTAT | |
| | TGATAGTACTCTTGGTTTTTATACCATTCAGATCACTGAATTTATAAA | |
| | GTACCCATCTAGTACTTGAAAAAGTAAAGTGTTCTGCCAGATCTTAG | |
| | GTATAGAGGACCCTAACACAGTATATCCCAAGTGCACTTTCTAATGT | |
| | TTCTGGGTCCTGAAGAATTAAGATACAAATTAATTTTACTCCATAAA | |
| | CAGACTGTTAATTATAGGAGCCTTAATTTTTTTTTCATAGAGATTTGT | |
| | CTAATTGCATCTCAAAATTATTCTGCCCTCCTTAATTTGGGAAGGTTT | |
| | GTGTTTTCTCTGGAATGGTACATGTCTTCCATGTATCTTTTGAACTGG | |
| | CAATTGTCTATTTATCTTTTATTTTTTTAAGTCAGTATGGTCTAACACT | |
| | GGCATGTTCAAAGCCACATTATTTCTAGTCCAAAATTACAAGTAATC | |
| | AAGGGTCATTATGGGTTAGGCATTAATGTTTCTATCTGATTTTGTGCA | |
| | AAAGCTTCAAATTAAAACAGCTGCATTAGAAAAAGAGGCGCTTCTC | |
| | CCCTCCCCTACACCTAAAGGTGTATTTAAACTATCTTGTGTGATTAAC | |
| | TTATTTAGAGATGCTGTAACTTAAAATAGGGGATATTTAAGGTAGCT | |
| | TCAGCTAGCTTTTAGGAAAATCACTTTGTCTAACTCAGAATTATTTTT | |
| | AAAAAGAAATCTGGTCTTGTTAGAAAACAAAATTTTATTTTGTGCTC | |
| | ATTTAAGTTTCAAACTTACTATTTTGACAGTTATTTTGATAACAATGA | |
| | CACTAGAAAACTTGACTCCATTTCATCATTGTTTCTGCATGAATATCA | |
| | TACAAATCAGTTAGTTTTTAGGTCAAGGGCTTACTATTTCTGGGTCTT | |
| | TTGCTACTAAGTTCACATTAGAATTAGTGCCAGAATTTTAGGAACTT | |
| | CAGAGATCGTGTATTGAGATTTCTTAAATAATGCTTCAGATATTATT | |
| | GCTTTATTGCTTTTTTGTATTGGTTAAAACTGTACATTTAAAATTGCT | |
| | ATGTTACTATTTTCTACAATTAATAGTTTGTCTATTTTAAAATAAATT | |
| | AGTTGTTAAGAGTCTTAA | |

Breast Cancer

Subjects with breast cancer tumors that fit in the Luminal A or Basal-like subtype, classified by gene expression analysis, were surprisingly found to have a significantly decreased rate of local recurrence and significantly increased rate of breast cancer specific survival when treated with a post-mastectomy breast cancer treatment that included radiation.

Classifying breast cancer tumors by intrinsic subtype and treating patients with radiation only when this treatment provides increased therapeutic efficacy to offset the added cost and side effects can improve the clinical outcome and quality of life of thousands of patients.

For the purposes of the present disclosure, “breast cancer” includes, for example, those conditions classified by biopsy or histology as malignant pathology. The clinical delineation of breast cancer diagnoses is well known in the medical arts. One of skill in the art will appreciate that breast cancer refers to any malignancy of the breast tissue, including, for example, carcinomas and sarcomas. Particular embodiments of breast cancer include ductal carcinoma in situ (DCIS), lobular carcinoma in situ (LCIS), or mucinous carcinoma. Breast cancer also refers to infiltrating ductal carcinoma (IDC), lobular neoplasia or infiltrating lobular carcinoma (ILC). In most embodiments of the disclosure, the subject of interest is a human patient suspected of or actually diagnosed with breast cancer.

Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can be in a pre-mastectomy female subject or a post-mastectomy female patient.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has been typed as being hormone receptor negative (e.g., estrogen receptor-negative) or hormone receptor positive status (e.g., estrogen receptor-positive). A breast cancer that is to be treated can include a tumor of the breast that has metastasized to other locations in the body. A breast cancer that is to be treated can be classified as having metastasized to a location selected from the group consisting of bone, lung, liver, lymph nodes, and brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

For the purposes of the present disclosure, “a breast cancer treatment comprising radiation” is a breast cancer treatment that includes radiation therapy, radiation treatment or radiation exposure. A “breast cancer treatment comprising radiation” can also be a breast cancer treatment that includes other anti-cancer or chemotherapeutic agents.

For the purposes of the present disclosure, “a breast cancer treatment not comprising radiation” is a breast cancer treatment that does not include any radiation therapy, radiation treatment or radiation exposure. These treatments can contain other anti-cancer or chemotherapeutic agents.

By “prolong” is meant an increase in time relative to a reference, standard, or control condition. Time may be increased anywhere from 0.01% to 10,000%, e.g., 0.01%, 0.05%, 0.1%, 0.5%, 1%, 2%, 3%, 4%, 5%, 6%, 7%, 8%, 9%, 10%, 20%, 25%, 30%, 40%, 50%, 60%, 70%, 75%, 80%, 90%, 100%, 200%, 300%, 400%, 500%, 600%, 700%, 800%, 900%, 1,000%, 2,000%, 3,000%, 4,000%, 5,000%, 6,000%, 7,000%, 8,000%, 9,000%, and 10,000%.

The amount of radiation used in radiation therapy (e.g., photon radiation therapy) is measured in gray (Gy), and varies depending on the type and stage of cancer being treated. The total dose of radiation therapy can be between about 20 to about 80 Gy. A dose for a solid epithelial tumor ranges can be from about 60 to about 80 Gy. A dose for lymphomas can be from about 20 Gy to about 40 Gy. Preventative (adjuvant) doses can be about 40 Gy to about 60 Gy. Preferably, about 45 Gy to about 60 Gy. Preferably, radiation therapy is administered in about 1.5 Gy to about 2.0 Gy fractions.

The total dose is fractionated (spread out over time), which permits normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given. One fractionation schedule for adults can be about 1.8 to about 2.0 Gy per day, five days a week. One fractionation schedule for children can be about 1.5 to about 1.8 Gy per day.

Accelerated Partial Breast Irradiation (APBI) is another fraction schedule use to treat breast cancer. APBI can be performed with either brachytherapy or with external beam radiation. APBI normally involves two high-dose fractions per day for five days, compared to whole breast irradiation, in which a single, smaller fraction is given five times a week over a six-to-seven-week period.

Classes of anti-cancer or chemotherapeutic agents can include anthracycline agents, alkylating agents, nucleoside analogs, platinum agents, taxanes, vinca agents, anti-estrogen drugs, aromatase inhibitors, ovarian suppression agents, endocrine/hormonal agents, bisphophonate therapy agents and targeted biological therapy agents.

Specific anti-cancer or chemotherapeutic agents can include cyclophosphamide, fluorouracil (or 5-fluorouracil or 5-FU), methotrexate, thiotepa, carboplatin, cisplatin, anthracyclines, gemcitabine, taxanes, paclitaxel, protein-bound paclitaxel, docetaxel, vinorelbine, tamoxifen, raloxifene, toremifene, fulvestrant, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb or bevacizumab, or combinations thereof; one such combination is CMF which includes cyclophosphamide, methotrexate, and fluorouracil.

Description of Intrinsic Subtype Biology

Luminal subtypes: The most common subtypes of breast cancer are the luminal subtypes, Luminal A and Luminal B. Prior studies suggest that Luminal A comprises approximately 30% to 40% and Luminal B approximately 20% of all breast cancers, but they represent over 90% of hormone receptor positive breast cancers (Nielsen et al. Clin. Cancer Res., 16(21):5222-5232 (2009)). The gene expression pattern of these subtypes resembles the luminal epithelial component of the breast. These tumors are characterized by high expression of estrogen receptor (ER), progesterone receptor (PR), and genes associated with ER activation, such as LIV1, GATA3, and cyclin D1, as well as expression of luminal cytokeratins 8 and 18 (Lisa Carey & Charles Perou (2009). “Gene Arrays, Prognosis, and Therapeutic Interventions”. Jay R. Harris et al. (4th ed.), “Diseases of the breast” (pp. 458-472). Philadelphia, Pa.: Lippincott Williams & Wilkins).

Luminal A: Luminal A (LumA) breast cancers exhibit low expression of genes associated with cell cycle activation and the ERBB2 cluster resulting in a better prognosis than Luminal B. The Luminal A subgroup has the most favorable prognosis of all subtypes and is enriched for endocrine therapy-responsive tumors.

Luminal B: Luminal B (LumB) breast cancers also express ER and ER-associated genes. Genes associated with cell cycle activation are highly expressed and this tumor type can be HER2(+) (˜20%) or HER2(−). The prognosis is unfavorable (despite ER expression) and endocrine therapy responsiveness is generally diminished relative to LumA.

HER2-enriched: The HER2-enriched subtype is generally ER-negative and is HER2-positive in the majority of cases with high expression of the ERBB2 cluster, including ERBB2 and GRB7. Genes associated with cell cycle activation are highly expressed and these tumors have a poor outcome.

Basal-like: The Basal-like subtype is generally ER-negative, is almost always clinically HER2-negative and expresses a suite of “Basal” biomarkers including the basal epithelial cytokeratins (CK) and epidermal growth factor receptor (EGFR). Genes associated with cell cycle activation are highly expressed.

Clinical Variables

The methods described herein, e.g., the PAM50 or NANO46 classification models, may be further combined with information on clinical variables (also referred to herein as “clinicopathological variables”) to generate a continuous risk of recurrence (ROR) predictor. As described herein, a number of clinical and prognostic breast cancer factors are known in the art and are used to predict treatment outcome and the likelihood of disease recurrence. Such factors include, for example, lymph node involvement, tumor size, histologic grade, estrogen and progesterone hormone receptor status, HER2 levels, and tumor ploidy. In one embodiment, risk of recurrence (ROR) score is provided for a subject diagnosed with or suspected of having breast cancer. This score uses an above-described classification model, e.g., the PAM50 or NANO46 classification models, in combination with clinical factors of lymph node status (N) and tumor size (T). Assessment of clinical variables is based on the American Joint Committee on Cancer (AJCC) standardized system for breast cancer staging. In this system, primary tumor size is categorized on a scale of 0-4 (T0: no evidence of primary tumor; T1: <2 cm; T2: >2 cm to <5 cm; T3: >5 cm; T4: tumor of any size with direct spread to chest wall or skin). Lymph node status is classified as N0-N3 (NO: regional lymph nodes are free of metastasis; N1: metastasis to movable, same-side axillary lymph node(s); N2: metastasis to same-side lymph node(s) fixed to one another or to other structures; N3: metastasis to same-side lymph nodes beneath the breastbone). Methods of identifying breast cancer patients and staging the disease are well known and may include manual examination, biopsy, review of patient's and/or family history, and imaging techniques, such as mammography, magnetic resonance imaging (MRI), and positron emission tomography (PET).

Sample Source

In one embodiment of the present disclosure, breast cancer subtype is assessed through the evaluation of expression patterns, or profiles, of the intrinsic genes listed in Table 1 in one or more subject samples and/or fluorescence in situ hybridization (FISH) analysis or immunohistochemistry (IHC) performed to ascertain the HER2 status of the cancer. As used herein, the term “subject” or “subject sample”, refers to an individual regardless of health and/or disease status. A subject can be a subject, a study participant, a control subject, a screening subject, or any other class of individual from whom a sample is obtained and assessed in the context of the disclosure. Accordingly, a subject can be diagnosed with breast cancer, can present with one or more symptoms of breast cancer, or a predisposing factor, such as a family (genetic) or medical history (medical) factor, for breast cancer, can be undergoing treatment or therapy for breast cancer, or the like. As such, the subject is a subject in need of treatment for breast cancer, detection of breast cancer, classification of a cancer, screening of likelihood of effectiveness of a treatment, and prediction of local-regional relapse free or breast cancer specific survival in response to a treatment. Alternatively, a subject can be healthy with respect to any of the aforementioned factors or criteria. It will be appreciated that the term “healthy” as used herein, is relative to breast cancer status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more cancers other than breast cancer. However, the healthy controls are preferably free of any cancer.

As used herein, a “subject in need thereof” is a subject having breast cancer or presenting with one or more symptoms of breast cancer, or a subject having an increased risk of developing breast cancer relative to the population at large. Preferably, a subject in need thereof has breast cancer. The breast cancer can be primary breast cancer, locally advanced breast cancer or metastatic breast cancer. A “subject” includes a mammal. The mammal can be any mammal, e.g., a human, a primate, a bird, a mouse, a rat, a fowl, a dog, a cat, a cow, a horse, a goat, a camel, a sheep and a pig. Preferably, the mammal is a human. The subject can be a male or a female.

In particular embodiments, the methods and kits for predicting breast cancer intrinsic subtypes or HER2 status (e.g., for predicting local-regional relapse free or breast cancer specific survival in a subject, for screening for the likelihood of the effectiveness of a post-mastectomy breast cancer treatment, and for treating breast cancer in a subject) include collecting a biological sample comprising a cancer cell or tissue, such as a breast tissue sample or a primary breast tumor tissue sample. By “biological sample” is intended any sampling of cells, tissues, or bodily fluids in which expression of an intrinsic gene can be detected. Examples of such biological samples include, but are not limited to, biopsies and smears. Bodily fluids useful in the present disclosure include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumor tissue sample. Biological samples may be obtained from a subject by a variety of techniques including, for example, by scraping or swabbing an area, by using a needle to aspirate cells or bodily fluids, or by removing a tissue sample (i.e., biopsy). Methods for collecting various biological samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Biological samples, particularly breast tissue samples, may be transferred to a glass slide for viewing under magnification. In one embodiment, the biological sample is a formalin fixed paraffin embedded (FFPE) breast tissue sample, particularly a primary breast tumor sample. In various embodiments, the tissue sample is obtained from a pathologist-guided tissue core sample.

Expression Profiling

In various embodiments, the present disclosure provides methods for classifying, prognosticating, or monitoring breast cancer in subjects. In this embodiment, data obtained from analysis of intrinsic gene expression is evaluated using one or more pattern recognition algorithms. See, as examples, U.S. Patent Application Publication Nos. 2011/0145176 and 2013/0337444. Such analysis methods may be used to form a predictive model, which can be used to classify test data. For example, one convenient and particularly effective method of classification employs multivariate statistical analysis modeling, first to form a model (a “predictive mathematical model”) using data (“modeling data”) from samples of known subtype (e.g., from subjects known to have a particular breast cancer intrinsic subtype: LumA, LumB, Basal-like, HER2-enriched, or normal-like), and second to classify an unknown sample (e.g., “test sample”) according to subtype. Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyze data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm.

The other approach is termed “supervised” whereby a training set of samples with known class or outcome is used to produce a mathematical model which is then evaluated with independent validation data sets. Here, a “training set” of intrinsic gene expression data is used to construct a statistical model that predicts correctly the “subtype” of each sample. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic gene expression profile. It is also possible to obtain confidence limits on any predictions, for example, a level of probability to be placed on the goodness of fit. The robustness of the predictive models can also be checked using cross-validation, by leaving out selected samples from the analysis.

The PAM50 or NANO46 classification models described herein (and as described in U.S. Patent Application Publication Nos. 2011/0145176 and 2013/0337444) is based on the gene expression profile for a plurality of subject samples using the 50 or 46, respectively, intrinsic genes listed in Table 1. The plurality of samples includes a sufficient number of samples derived from subjects belonging to each subtype class. By “sufficient samples” or “representative number” in this context is intended a quantity of samples derived from each subtype that is sufficient for building a classification model that can reliably distinguish each subtype from all others in the group. A supervised prediction algorithm is developed based on the profiles of objectively-selected prototype samples for “training” the algorithm. The samples are selected and subtyped using an expanded intrinsic gene set according to the methods disclosed in International Patent Publication WO 2007/061876 and U.S. Patent Publication No. 2009/0299640. Alternatively, the samples can be subtyped according to any known assay for classifying breast cancer subtypes. After stratifying the training samples according to subtype, a centroid-based prediction algorithm is used to construct centroids based on the expression profile of all or some of the intrinsic gene set described in Table 1.

In one embodiment, the prediction algorithm is the nearest centroid methodology related to that described in Narashiman and Chu (2002) PNAS 99:6567-6572. In the present disclosure, the method computes a standardized centroid for each subtype. This centroid is the average gene expression for each gene in each subtype (or “class”) divided by the within-class standard deviation for that gene. Nearest centroid classification takes the gene expression profile of a new sample, and compares it to each of these class centroids. Subtype prediction is done by calculating the Spearman's rank correlation of each test case to the five centroids, and assigning a sample to a subtype based on the nearest centroid.

Detection of Intrinsic Gene Expression

Any methods available in the art for detecting expression of the intrinsic genes listed in Table 1 are encompassed herein. By “detecting expression” is intended determining the quantity or presence of an RNA transcript or its expression product of an intrinsic gene. Methods for detecting expression of the intrinsic genes of the disclosure, that is, gene expression profiling, include methods based on hybridization analysis of polynucleotides, methods based on sequencing of polynucleotides, immunohistochemistry methods, and proteomics-based methods. The methods generally detect expression products (e.g., mRNA) of the intrinsic genes listed in Table 1. In preferred embodiments, PCR-based methods, such as reverse transcription PCR (RT-PCR) (Weis et al., TIG 8:263-64, 1992), and array-based methods such as microarray (Schena et al., Science 270:467-70, 1995) are used. By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to an intrinsic gene. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as a tumor or tumor cell line, and corresponding normal tissue or cell line, respectively. If the source of RNA is a primary tumor, RNA (e.g., mRNA) can be extracted, for example, from frozen or archived paraffin-embedded and fixed (e.g., formalin-fixed) tissue samples (e.g., pathologist-guided tissue core samples).

General methods for RNA extraction are well known in the art and are disclosed in standard textbooks of molecular biology, including Ausubel et al., ed., “Current Protocols in Molecular Biology”, John Wiley & Sons, New York 1987-1999. Methods for RNA extraction from paraffin embedded tissues are disclosed, for example, in Rupp and Locker, Lab Invest. 56:A67, (1987); and De Andres et al. Biotechniques 18:42-44, (1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include Masterpure™ Complete DNA and RNA Purification Kit (Epicentre®, Madison, Wis.) and Paraffin Block RNA Isolation Kit (Ambion®, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to an intrinsic gene of the present disclosure, or any derivative DNA or RNA. Hybridization of an mRNA with the probe indicates that the intrinsic gene in question is being expressed. The term “stringent conditions” is as well-known in the art and as described, at least, in books, publications and patent documents listed herein.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent (Santa Clara, Calif.) gene chip array. A skilled artisan can readily adapt known mRNA detection methods for use in detecting the level of expression of the intrinsic genes of the present disclosure.

An alternative method for determining the level of intrinsic gene expression product in a sample involves the process of nucleic acid amplification, for example, by RT-PCR (U.S. Pat. No. 4,683,202), ligase chain reaction (Barany, PNAS USA 88: 189-93, (1991)), self-sustained sequence replication (Guatelli et al., PNAS USA 87: 1874-78, (1990)), transcriptional amplification system (Kwoh et al., PNAS USA 86: 1173-77, (1989)), Q-Beta Replicase (Lizardi et al., Bio/Technology 6:1197, (1988)), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers.

In particular aspects of the disclosure, intrinsic gene expression can assessed by quantitative RT-PCR. Numerous different PCR or quantitative real-time PCR (qPCR) protocols are known in the art and exemplified herein and can be directly applied or adapted for use using the presently-described methods and kits for the detection and/or quantification of the intrinsic genes listed in Table 1. Generally, in PCR, a target polynucleotide sequence is amplified by reaction with at least one oligonucleotide primer or a pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real time fluorescence measurement capabilities, for example, Smartcycler® (Cepheid, Sunnyvale, Calif.), ABI Prism 7700® (Applied Biosystems®, Foster City, Calif.), Rotor-Gene™ (Corbett Research, Sydney, Australia), Lightcycler® (Roche Diagnostics Corp, Indianapolis, Ind.), iCycler® (Biorad Laboratories, Hercules, Calif.) and MX4000® (Stratagene, La Jolla, Calif.).

In another embodiment of the disclosure, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample.

In a preferred embodiment, the nCounter® Analysis System (NanoString Technologies, Seattle, Wash.) is used to detect intrinsic gene expression. The basis of the nCounter® Analysis System is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system.

Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene of Table 1 and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene of Table 1 to be detected. Reporter and capture probes are all pooled into a single hybridization mixture, the “probe library”. Preferably, the probe library comprises a probe pair (a capture probe and reporter) for each of the genes in Table 1. Preferably, the probe library comprises a probe pair (a capture probe and reporter) for each of the NANO46 genes as described above. Preferably, the probe library comprises a probe pair (a capture probe and reporter) for each of the housekeeping genes and other genes described herein, e.g., Her2.

The relative abundance of each target is measured in a single multiplexed hybridization reaction. The method comprises contacting a biological sample with a probe library, the library comprising a probe pair for each of the at least 40 genes in Table 1, e.g., each of the NANO46 or PAM50 genes, and/or the housekeeping genes and other genes described herein, such that the presence of each target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies).

Purified reactions are deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm² of the binding surface. Typical imaging density is 100-1200 counted reporters per field of view depending on the degree of multiplexing, the amount of sample input, and overall target abundance. Data is output in simple spreadsheet format listing the number of counts per target, per sample.

This system can be used along with nanoreporters. Additional disclosure regarding nanoreporters can be found in International Publication No. WO 07/076129 and WO 07/076132, and US Patent Publication No. 2010/0015607 and 2010/0261026. Further, the term nucleic acid probes and nanoreporters can include the rationally designed (e.g., synthetic sequences) described in International Publication No. WO 2010/019826 and US Patent Publication No. 2010/0047924.

Data Processing

It is often useful to pre-process gene expression data, for example, by addressing missing data, translation, scaling, normalization, and weighting. Multivariate projection methods, such as principal component analysis (PCA) and partial least squares analysis (PLS), are so-called scaling sensitive methods. By using prior knowledge and experience about the type of data studied, the quality of the data prior to multivariate modeling can be enhanced by scaling and/or weighting. Adequate scaling and/or weighting can reveal important and interesting variation hidden within the data, and therefore make subsequent multivariate modeling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may be replaced or “filled” with, for example, the mean value of a column (“mean fill”); a random value (“random fill”); or a value based on a principal component analysis (“principal component fill”).

“Translation” of the descriptor coordinate axes can be useful. Examples of such translation include normalization and mean centering. “Normalization” may be used to remove sample-to-sample variation. For microarray data, the process of normalization aims to remove systematic errors by balancing the fluorescence intensities of the two labeling dyes. The dye bias can come from various sources including differences in dye labeling efficiencies, heat and light sensitivities, as well as scanner settings for scanning two channels. Some commonly used methods for calculating normalization factor include: (i) global normalization that uses all genes on the array; (ii) housekeeping genes normalization that uses constantly expressed housekeeping/invariant genes; and (iii) internal controls normalization that uses known amount of exogenous control genes added during hybridization (Quackenbush, Nat. Genet. 32 (Suppl.), 496-501 (2002)). In one embodiment, the intrinsic genes disclosed herein can be normalized to control housekeeping genes. For example, the housekeeping genes described in U.S. Patent Publication 2008/0032293 can be used for normalization. Exemplary housekeeping genes include MRPL19, PSMC4, SF3A1, PUM1, ACTB, GAPD, GUSB, RPLPO, and TFRC. It will be understood by one of skill in the art that the methods disclosed herein are not bound by normalization to any particular housekeeping genes, and that any suitable housekeeping gene(s) known in the art can be used.

Many normalization approaches are possible, and they can often be applied at any of several points in the analysis. In one embodiment, microarray data is normalized using the LOWESS method, which is a global locally weighted scatterplot smoothing normalization function. In another embodiment, qPCR data is normalized to the geometric mean of set of multiple housekeeping genes.

“Mean centering” may also be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In Pareto scaling, the value of each descriptor is scaled by 1/sqrt(StDev), where StDev is the standard deviation for that descriptor for all samples. In this way, each descriptor has a variance numerically equal to its initial standard deviation. The Pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is very useful because each descriptor is then weighted equally, and large and small values are treated with equal emphasis. This can be important for genes expressed at very low, but still detectable, levels.

In one embodiment, data is collected for one or more test samples and classified using the at least 40 genes of Table 1 as described herein, e.g., the PAM50 or NANO46 classification models. When comparing data from multiple analyses (e.g., comparing expression profiles for one or more test samples to the centroids constructed from samples collected and analyzed in an independent study), it will be necessary to normalize data across these data sets. In one embodiment, Distance Weighted Discrimination (DWD) is used to combine these data sets together (Benito et al. (2004) Bioinformatics 20(1): 105-114). DWD is a multivariate analysis tool that is able to identify systematic biases present in separate data sets and then make a global adjustment to compensate for these biases; in essence, each separate data set is a multi-dimensional cloud of data points, and DWD takes two points clouds and shifts one such that it more optimally overlaps the other.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, non-transitory computer-readable media, and other memory and computer storage devices. The computer program that may be used to configure the computer to carry out the steps of the methods and/or record the results may also be provided over an electronic network, for example, over the internet, an intranet, or other network.

Calculation of Risk of Recurrence

Provided herein are methods for predicting breast cancer outcome within the context of the intrinsic subtype and optionally other clinical variables. Outcome may refer to overall or disease-specific survival, event-free survival, or outcome in response to a particular treatment or therapy. In particular, the methods may be used to predict the likelihood of long-term, disease-free survival. “Predicting the likelihood of survival of a breast cancer patient” is intended to assess the risk that a patient will die as a result of the underlying breast cancer. “Long-term, disease-free survival” is intended to mean that the patient does not die from or suffer a recurrence of the underlying breast cancer within a period of at least five years, or at least ten or more years, following initial diagnosis or treatment.

In embodiments, outcome is predicted based on classification of a subject according to cancer subtype. This classification is based on expression profiling using the at least 40 intrinsic genes listed in Table 1. In addition to providing a subtype assignment, the at least 40 intrinsic genes listed in Table 1, e.g., the PAM50 or NANO46 genes, provide measurements of the similarity of a test sample to all four subtypes which is translated into a Risk of Recurrence (ROR) score that can be used in any patient population regardless of disease status and treatment options. The intrinsic subtypes and ROR also have value in the prediction of pathological complete response in women treated with, for example, neoadjuvant taxane and anthracycline chemotherapy (Rouzier et al., J Clin Oncol 23:8331-9 (2005)). Thus, in various embodiments of the present disclosure, a risk of recurrence (ROR) model is used to predict outcome. Using these risk models, subjects can be stratified into low, medium, and high risk of recurrence groups. Calculation of ROR can provide prognostic information to guide treatment decisions and/or monitor response to therapy.

In some embodiments described herein, the prognostic performance of the intrinsic subtypes defied by expression profiles of the at least 40 genes listed in Table 1, e.g., the PAM50- or NANO46-defined intrinsic subtypes, and/or other clinical parameters is assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., intrinsic gene expression profile with or without additional clinical factors, as described herein). The “hazard ratio” is the risk of death at any given time point for patients displaying particular prognostic variables. See generally Spruance et al., Antimicrob. Agents & Chemo. 48:2787-92 (2004).

The classification models described herein, e.g., the PAM50 or NANO46 classification models, can be trained for risk of recurrence using subtype distances (or correlations) alone, or using subtype distances with clinical variables as discussed supra. In one embodiment, the risk score for a test sample is calculated using intrinsic subtype distances alone using the following equation (Equation 2):

ROR=0.05*Basal+0.1 l*HER2+−0.25*LumA+0.07*LumB+−0.1 l*Normal,
where the variables “Basal,” “HER2,” “LumA,” “LumB,” and “Normal” are the distances to the centroid for each respective classifier when the expression profile from a test sample is compared to centroids constructed using the gene expression data deposited with the National Center for Biotechnology Information Gene Expression Omnibus (GEO); as examples with accession number GSE2845 or GSE10886.

Risk score can also be calculated using a combination of breast cancer subtype and the clinical variables tumor size (T) and lymph nodes status (N) using the following equation (Equation 3):

ROR(full)=0.05*Basal+0.1*HER2+−0.19*LumA+0.05*LumB+−0.09*Normal+0.16*T+0.08*N,
where the variables “Basal,” “HER2,” “LumA,” and “LumB” are as described supra and when comparing test expression profiles to centroids constructed using the gene expression data deposited with GEO; as examples with accession number GSE2845 or GSE10886.

In yet another embodiment, risk score for a test sample is calculated using intrinsic subtype distances alone using the following equation (Equation 4):

ROR-S=0.05*Basal+0.12*HER2+−0.34*LumA+0.0.23*LumB,
where the variables “Basal,” “HER2,” “LumA,” and “LumB” are as described supra and the test expression profiles are compared to centroids constructed using the gene expression data deposited with GEO; as examples with accession number GSE2845 or GSE10886.

In yet another embodiment, risk score can also be calculated using a combination of breast cancer subtype and the clinical variable tumor size (T) using the following equation (Equation 5):

ROR-C=0.05*Basal+0.1 l*HER2+−0.23*LumA+0.09*LumB+0.17*T,
where the variables “Basal,” “HER2,” “LumA,” and “LumB” are as described supra and the test expression profiles are compared to centroids constructed using the gene expression data deposited with GEO; as examples with accession number GSE2845 or GSE10886.

In yet another embodiment, risk score for a test sample is calculated using intrinsic subtype distances in combination with the proliferation signature (“Prolif”) using the following equation (Equation 6):

ROR-P=−0.001*Basal+0.7*HER2+−0.95*LumA+0.49*LumB+0.34*Prolif,
where the variables “Basal,” “HER2,” “LumA,” “LumB” and “Prolif” are as described supra and the test expression profiles are compared to centroids constructed using the gene expression data deposited with GEO; as examples with accession number GSE2845 or GSE10886.

In yet another embodiment, risk score can also be calculated using a combination of breast cancer subtype, proliferation signature and the clinical variable tumor size (T) using the ROR-PT described in conjunction with Table 5, supra.

Detection of Subtypes

Immunohistochemistry (IHC) for estrogen receptor (ER), progesterone receptor (PR), HER2, and Ki67 can be performed concurrently on serial sections with the standard streptaviding biotin complex method with 3,3′-diaminobenzidine as the chromogen. Staining for ER, PR, and HER2 interpretation can be performed as described previously (Cheang et al., Clin Cancer Res. 2008; 14(5):1368-1376), however any method known in the art may be used.

For example, a Ki67 antibody (clone SP6; ThermoScientific™, Fremont, Calif.) can be applied at a 1:200 dilution for 32 minutes, by following the Ventana Benchmark automated immunostainer (Ventana®, Tucson, Ariz.) standard Cell Conditioner 1 (CC1, a proprietary buffer) protocol at 98° C. for 30 minutes. An ER antibody (clone SP1; ThermoFisher Scientific™) can be used at 1:250 dilution with 10-minute incubation, after an 8-minute microwave antigen retrieval in 10 mM sodium citrate (pH 6.0). Ready-to-use PR antibody (clone 1E2; Ventana®) can be used by following the CC1 protocol as above. HER2 staining can be done with a SP3 antibody (ThermoFisher Scientific™) at a 1:100 dilution after antigen retrieval in 0.05 M Tris buffer (pH 10.0) with heating to 95° C. in a steamer for 30 minutes. For HER2 fluorescent in situ hybridization (FISH) assay, slides can be hybridized with probes to LSI (locus-specific identifier) HER2/neu and to centromere 17 by use of the PathVysion HER-2 DNA Probe kit (Abbott Molecular, Abbott Park, Ill.) according to manufacturer's instructions, with modifications to pretreatment and hybridization as previously described (Brown L A, Irving J, Parker R, et al. “Amplification of EMSY, a novel oncogene on 11q13, in high grade ovarian surface epithelial carcinomas”. Gynecol Oncol. 2006; 100(2):264-270). Slides can then be counterstained with 4′,6-diamidino-2-phenylindole. Stained material can be visualized on a Zeiss Axioplan epifluorescent microscope, and signals analyzed with a Metafer image acquisition system (Metasystems, Altlussheim, Germany). Biomarker expression from immunohistochemistry assays can then be scored by two pathologists, who are blinded to the clinicopathological characteristics and outcome and who used previously established and published criteria for biomarker expression levels that had been developed on other breast cancer cohorts.

Tumors are considered positive for ER or PR if immunostaining is observed in more than 1% of tumor nuclei, as described previously. Tumors are considered positive for HER2 if immunostaining is scored as 3+ according to HercepTest™ (Dako, Carpinteria, Calif.) criteria, with an amplification ratio for fluorescent in situ hybridization of 2.0 or more being the cut point that can be used to segregate immunohistochemistry equivocal tumors (scored as 2+) (Yaziji, et al., JAMA, 291(16):1972-1977 (2004)). Ki67 can be visually scored for percentage of tumor cell nuclei with positive immunostaining above the background level.

Other methods can also be used to detect the HER2+ subtype. These techniques include enzyme-linked immunosorbent assay (ELISA), Western blots, Northern blots, or fluorescence-activated cell sorting (FACS) analysis.

Kits

The present disclosure also describes kits useful for classifying breast cancer intrinsic subtypes and/or providing prognostic information to identify breast cancers that are more or less responsive to radiation. These kits comprise a set of reporter/capture probes and/or primers specific for the genes listed in Table 1, and/or housekeeping genes, and/or other genes descrbed herein. The kits can further include instructions for detecting the aforementioned genes and classifying breast cancer intrinsic subtypes and/or providing prognostic information to identify breast cancers that are more responsive to radiation. The kits may include instructions for recommended treatments based on a classified breast cancer intrinsic subtype. The kits may also contain reagents sufficient to facilitate detection and/or quantitation of HER2, in order to classify cells as HER2+. Preferably, the kit comprises a set of reporter/capture probes and/or primers specific for at least 10, at least 15, at least 20, at least 25, at least 40, 41, 42, 43, 44, 45, 46, 47, 48, 49 or all 50 genes listed in Table 1. The kit may further comprise a non-transitory computer readable medium.

In embodiments of the present disclosure, the capture probes are immobilized on an array. By “array” is intended a solid support or a substrate with peptide or nucleic acid probes attached to the support or substrate. Arrays typically comprise a plurality of different capture probes that are coupled to a surface of a substrate in different, known locations. The arrays of the disclosure comprise a substrate having a plurality of capture probes that can specifically bind an intrinsic gene expression product. The number of capture probes on the substrate varies with the purpose for which the array is intended. The arrays may be low-density arrays or high-density arrays and may contain 4 or more, 8 or more, 12 or more, 16 or more, 32 or more addresses, but will minimally comprise capture probes for at least 10, at least 15, at least 20, at least 25, or at least 46 of the intrinsic genes or all 50 intrinsic genes listed in Table 1. The array may include capture probes for the housekeeping genes and/or other genes listed herein.

Techniques for the synthesis of these arrays using mechanical synthesis methods are described in, e.g., U.S. Pat. No. 5,384,261. The array may be fabricated on a surface of virtually any shape or even a multiplicity of surfaces. Arrays may be probes (e.g., nucleic-acid binding probes) on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation on the device. See, for example, U.S. Pat. Nos. 5,856,174 and 5,922,591.

In embodiments, the kit comprises a set of oligonucleotide primers sufficient for the detection and/or quantitation of each of the intrinsic genes listed in Table 1. Preferably, the kit comprises a set of oligonucleotide primers sufficient for the detection and/or quantitation of at least 10, at least 15, at least 20, at least 25, at least 46 of the intrinsic genes or all 50 intrinsic genes listed in Table 1 and/or for the detection and/or quantitation of the housekeeping genes and/or other genes listed herein. The oligonucleotide primers may be provided in a lyophilized or reconstituted form, or may be provided as a set of nucleotide sequences. In certain embodiments, the primers are provided in a microplate format, where each primer set occupies a well (or multiple wells, as in the case of replicates) in the microplate. The microplate may further comprise primers sufficient for the detection of one or more housekeeping genes (e.g., eight) as discussed herein. The kit may further comprise reagents and instructions sufficient for the amplification of expression products from the genes listed in Table 1 and/or for the amplification of expression products from the housekeeping genes and/or other genes listed herein.

In order to facilitate ready access, e.g., for comparison, review, recovery, and/or modification, the molecular signatures/expression profiles are typically recorded in a database. Most typically, the database is a relational database accessible by a computational device, although other formats, e.g., manually accessible indexed files of expression profiles as photographs, analogue or digital imaging readouts, and spreadsheets can be used. Regardless of whether the expression patterns initially recorded are analog or digital in nature, the expression patterns, expression profiles (collective expression patterns), and molecular signatures (correlated expression patterns) are stored digitally and accessed via a database. Typically, the database is compiled and maintained at a central facility, with access being available locally and/or remotely.

In certain embodiments, the kit also includes a substance that is used to find the expression level of HER2. This substance can be an antibody or a nucleic acid probe. These substances can be used to detect HER2 using FISH, IHC, ELISA, Western blots, Northern blots, or FACS analysis. Optionally, the kit also includes reagents that allows for the detection of the detecting substance and the quantitation of HER2 expression in a sample.

### Examples

### Example 1

### Background

Luminal A (LumA) tumors are associated with good prognosis, but with substantial risk for late loco-regional relapses. Here was tested the predictive value of intrinsic subtypes as defined by research-based PAM50 classifier, for predicting adjuvant radiation therapy benefit among pre-menopausal women with node positive tumors from a post mastectomy randomized adjuvant radiation trials with more than 20 years follow-up.

Methods:

Formalin fixed paraffin embedded tissues (FFPE) (n=145) were collected from the British Columbia trial and gene expression profiles were done using Nanostring nCounter® for FFPE samples. Tumors were classified into subtypes (Luminal A (LumA), Luminal B (LumB), HER2-enriched (HER2-E), Basal-like (BLBC) and Normal-like) based on the PAM50 classifier. Kaplan-Meier analysis and the log-rank test were used to test the differences in local-regional relapse free survival (LRFS) and breast cancer specific survival (BCSS).

RNA can be extracted from Formalin-fixed, Paraffin-embedded (FFPE) tissue that has been diagnosed as having a carcinoma of the breast. A Pathologist reviews a hematoxylin and eosin stain (H & E) stained slide to identify the tissue area containing sufficient tumor tissue content for the test. Unstained slide mounted tissue sections are processed by macro-dissecting the identified tumor area on each slide to remove any adjacent normal tissue. RNA is then isolated from the tumor tissue, and DNA is removed from the sample.

Total RNA was extracted using the High Pure RNA Paraffin Kit (Roche Applied Science, Indianapolis, Ind., cat#03270289001), according to the manufacturer's protocol. RNA yield and purity were assessed using the NanoDrop ND-1000 Spectrophotometer (NanoDrop Technologies, Rockland, Del.). RNA samples used in downstream analysis met pre-specified quality criteria of an initial concentration of total RNA≧12.5 ng/μl, a minimum total yield of 250 ng, and a purity ratio in the range 1.7-2.5.

Gene expression was measured on the NanoString nCounter® Analysis System which delivers direct, multiplexed measurements through digital readouts of the relative abundance of hundreds of mRNA transcripts. In brief, the expression of the fifty target genes of Table 1 (PAM50) as well as normalizing “housekeeping” genes (for example MRPL19, PSMC4, SF3A1, PUM1, ACTB, GAPDH, GUSB, RPLPO, and TFRC) were measured in a single hybridization reaction without the use of any enzymatic reactions. An nCounter® CodeSet with gene-specific probe-pairs to the PAM50 targets as well as exogenous positive and negative controls was hybridized in solution to 125-500 ng total RNA (nominally 250 ng). After overnight hybridization, the samples were processed using the NanoString nCounter® Prep Station and Digital Analyzer according to the instructions and kits provided by NanoString Technologies. Data from each sample were qualified using prospectively defined quality control metrics for the positive and negative controls included in each reaction.

Intrinsic subtype classification of qualified patient samples was based upon the PAM50 gene expression signature. Reporter-code-count files, containing the digital abundance or “counts” of each target mRNA molecule for every sample, were sent to NanoString Technologies for PAM50 subtype calling using a prospectively defined and locked proprietary algorithm. Assignment of subtypes was performed in a blinded fashion, by researchers with no access to information regarding the clinical parameters or outcomes.

Results:

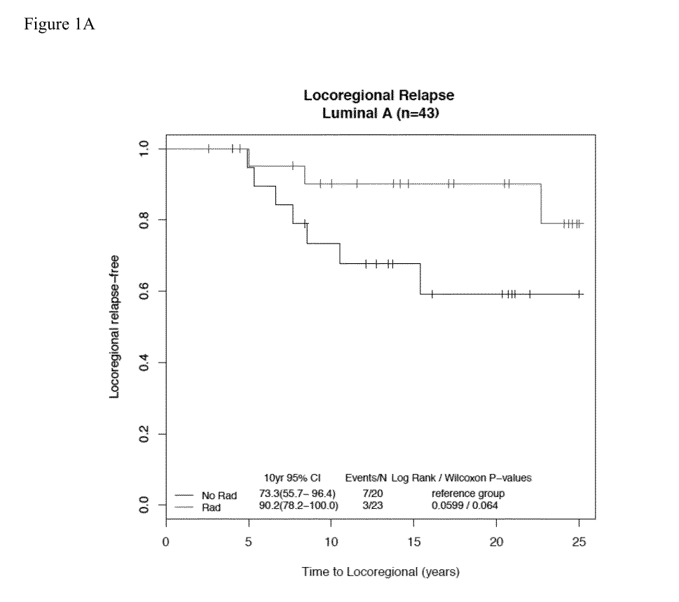

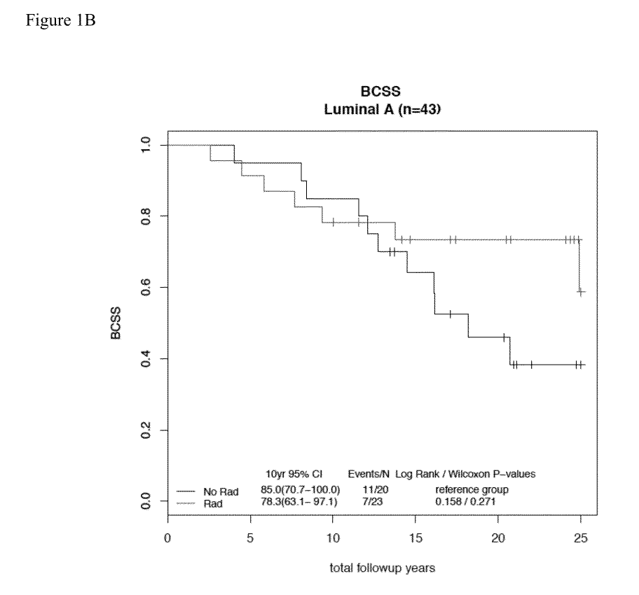

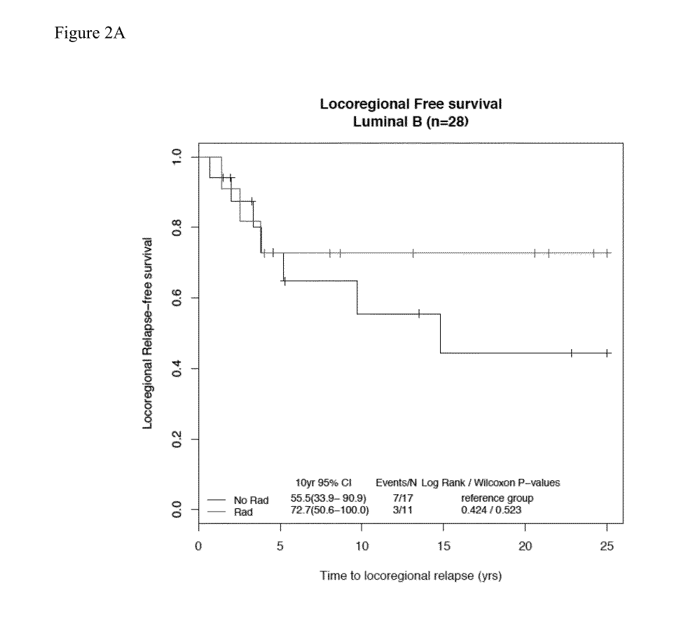

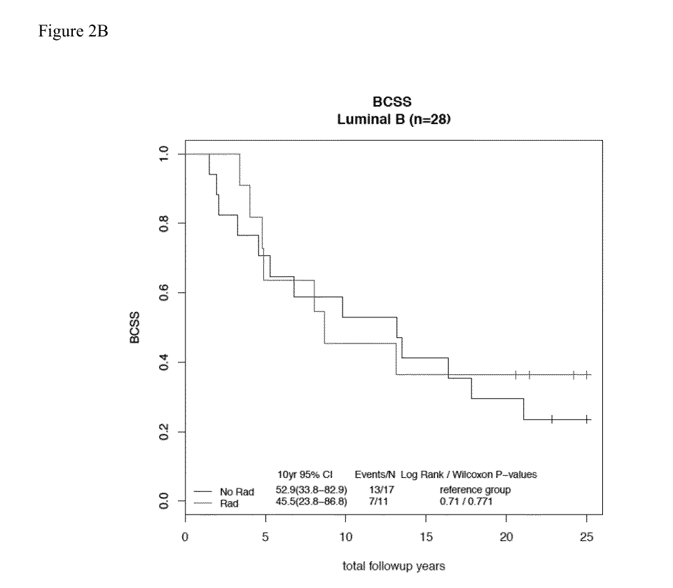

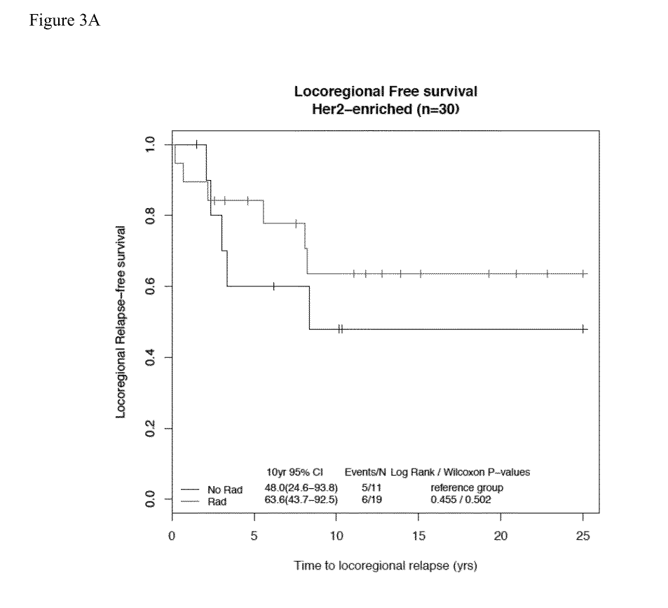

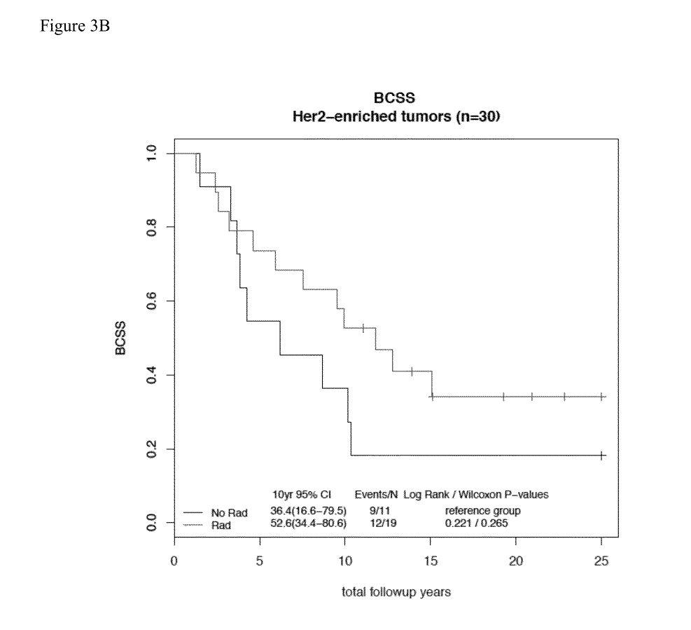

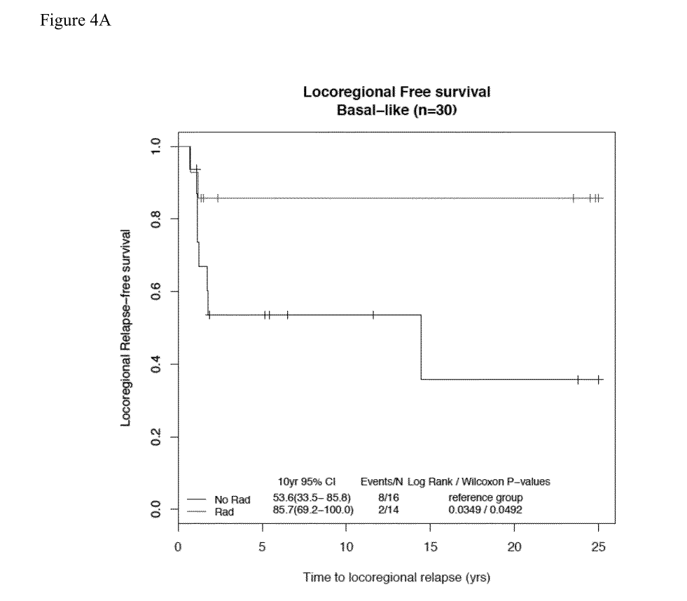

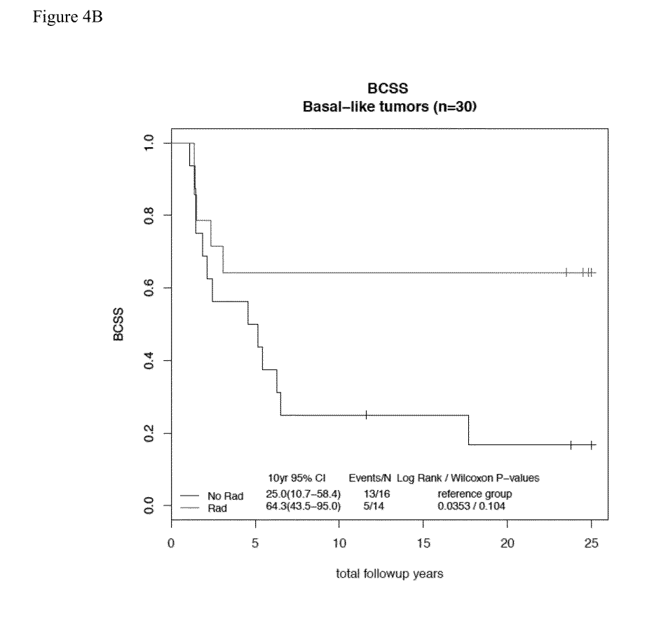

In this trial, patients received adjuvant CMF (cyclophosphamide, methotrexate, and fluorouracil) and were randomized to with or without post mastectomy radiation therapy (RT) groups. Patients with estrogen receptor positive tumor, as defined by the dextran charcoal biochemical assay, were randomized selected to receive oophorectomy and 42 of them were included in this correlative science study. FIG. 1A shows loco-regional relapse for subjects whose tumor samples are classified as Luminal A, with or without radiation therapy. FIG. 1B shows breast cancer specific survival (BCSS) for subjects whose tumor samples are classified as Luminal A, with or without radiation therapy. FIG. 2A shows loco-regional relapse for subjects whose tumor samples are classified as Luminal B, with or without radiation therapy. FIG. 2B shows breast cancer specific survival (BCSS) for subjects whose tumor samples are classified as Luminal B, with or without radiation therapy. FIG. 3A shows loco-regional relapse for subjects whose tumor samples are classified as HER2-enriched, with or without radiation therapy. FIG. 3B shows breast cancer specific survival (BCSS) for subjects whose tumor samples are classified as HER2-enriched, with or without radiation therapy. FIG. 4A shows loco-regional relapse for subjects whose tumor samples are classified as Basal-like, with or without radiation therapy. FIG. 4B shows breast cancer specific survival (BCSS) for subjects whose tumor samples are classified as Basal-like, with or without radiation therapy.

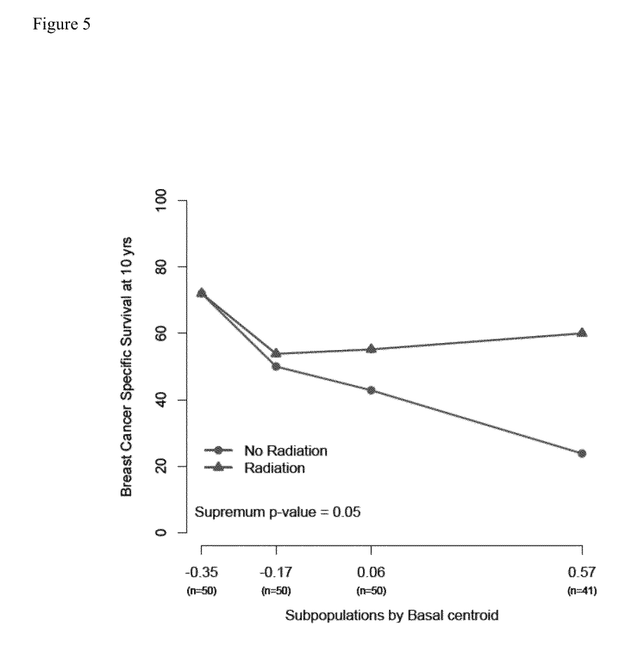

FIG. 5 shows a subpopulation treatment effect pattern plot (STEPP) showing 10-year breast cancer specific survival (BCSS) to the Spearman's correlation to Basal-like tumors average expression profile.

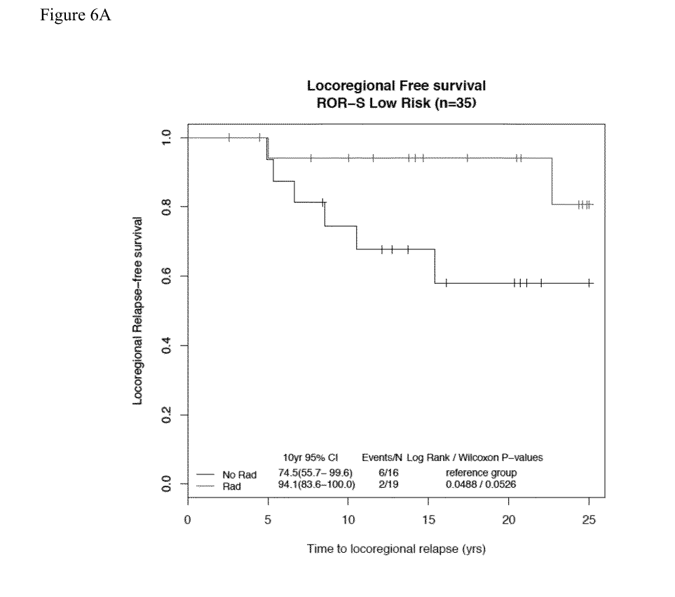

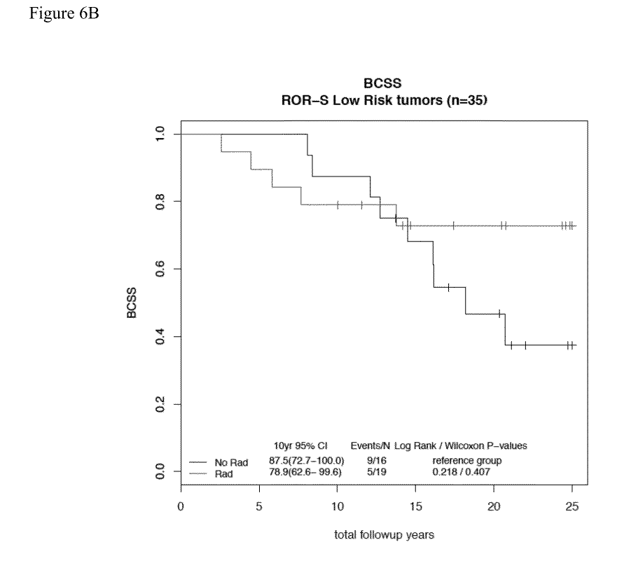

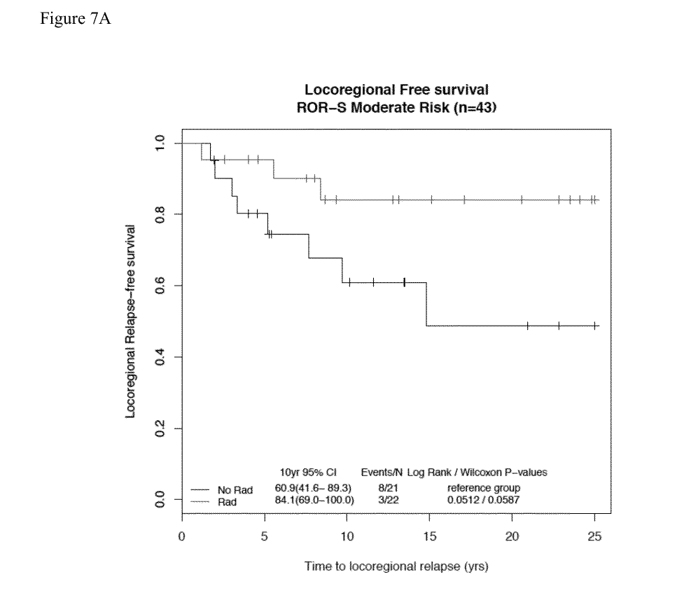

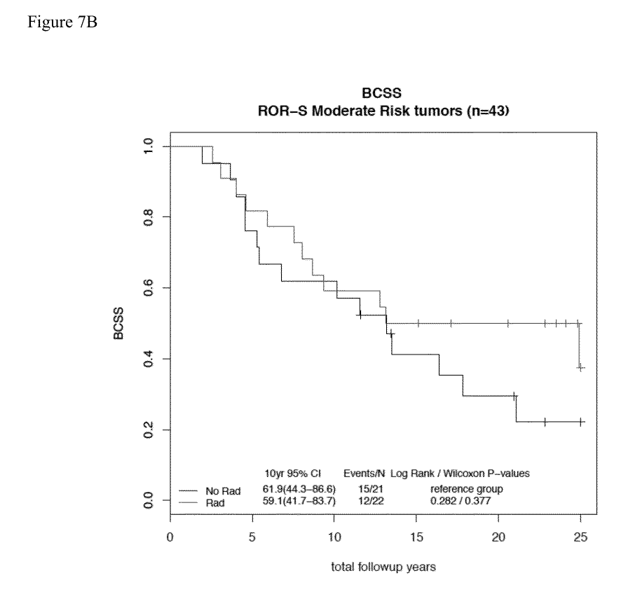

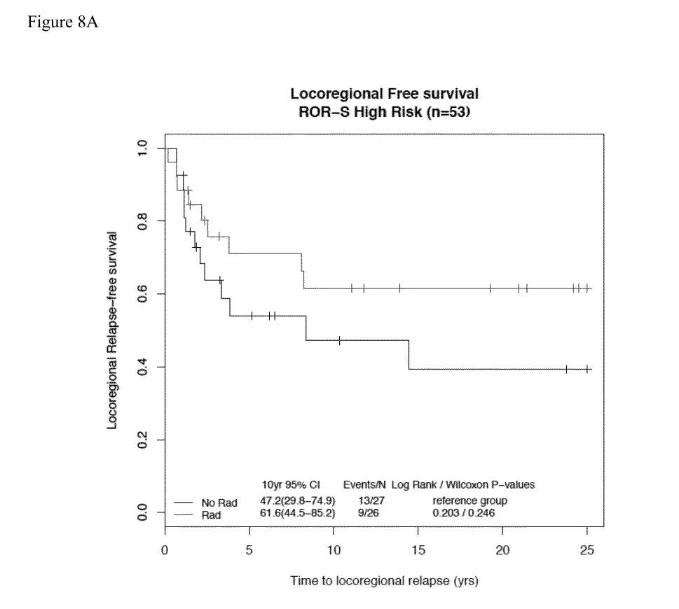

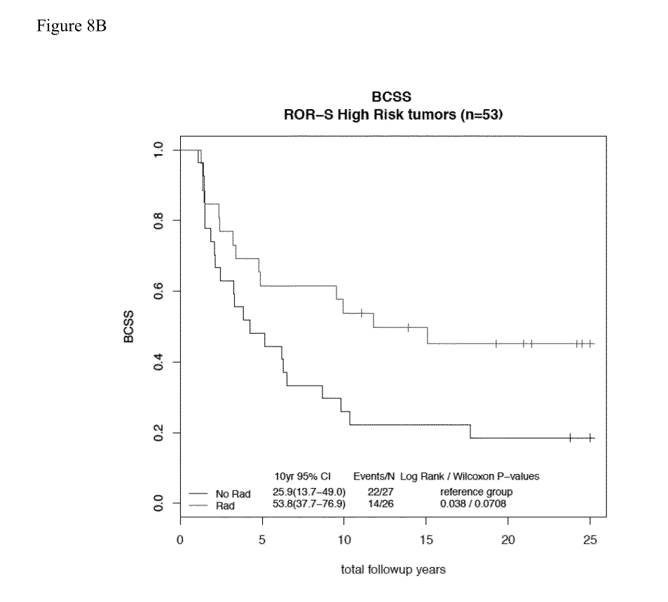

FIG. 6A shows loco-regional relapse for subjects who are classified as low risk based on their Risk of Recurrence Score (subtypes centroid based), ROR-S, with or without radiation therapy. FIG. 6B shows breast cancer specific survival (BCSS) for subjects who are classified as low risk based on their Risk of Recurrence Score (subtypes centroid based), ROR-S, with or without radiation therapy. FIG. 7A shows loco-regional relapse for subjects who are classified as moderate/intermediate risk based on their Risk of Recurrence Score (subtypes centroid based), ROR-S, with or without radiation therapy. FIG. 7B shows breast cancer specific survival (BCSS) for subjects who are classified as moderate/intermediate risk based on their Risk of Recurrence Score (subtypes centroid based), ROR-S, with or without radiation therapy. FIG. 8A shows loco-regional relapse for subjects who are classified as high risk based on their Risk of Recurrence Score (subtypes centroid based), ROR-S, with or without radiation therapy. FIG. 8B shows breast cancer specific survival (BCSS) for subjects who are classified as high risk based on their Risk of Recurrence Score (subtypes centroid based), ROR-S, with or without radiation therapy.

These results demonstrate improved breast cancer specific survival (BCSS) for tumor samples classified as Basal-like subtype and have classified as ROR-S high risk and also demonstrate improved loco-regional relapse survival for tumor samples classified as Luminial A subtype and classified as ROR-S low risk.

### Example 2

Herein an aim was to investigate the predictive value of additional genomic profiles (continuous measurements instead of subgroup analysis) for loco-regional recurrences (LRR) and breast cancer survival (BCSS) in node-positive, pre-menopausal breast cancer patients randomized to adjuvant chemoradiation or chemotherapy alone, in the British Columbia trial.

Methods: In the British Columbia trial, 318 patients received adjuvant cyclophosphamide, methotrexate, fluorouracil (CMF) and were randomized to with or without postmastectomy RT groups. From 145 formalin fixed paraffin embedded tissues, expression profiling of 66 genes was done with the Nanostring nCounter® Subpopulation Treatment Effect Pattern Plot analysis and permutation tests were used to examine treatment effects on LRR and BCSS events for the absolute difference (Kaplan-Meier) and relative effectiveness (Hazard Ratio) terms. For each tumor, the research-based PAM50 proliferation score, a Spearman's correlation to each of the four intrinsic subtypes (i.e., a quantitative measurement of similarity to the average expression profiles of a typical HER2-Enriched, Basal-like, Luminal A and Luminal B), Risk of Recurrence scores (ROR) and a 13-gene VEGF-signature score (VEGF-s) were calculated as previously described (Parker et al, J. Clin. Oncol., 27(8):1160-7 (2009); Hu et at BMC Medicine, 7:9 2009). Expression level of DNA repair genes (RAD17 and RAD50) and tumor suppressor RB1 were also measured.

Results: Overall, patients in the RT arm (n=69) were significantly associated with better LRR and BCSS than the non-RT-treated arm (n=76). No significant treatment-effect heterogeneity was detected for VEGF-s, RAD17 and RAD50 expressions. On the other hand, patients with lower RB1 expression levels and higher proliferation scores had better LRR survival when assigned the RT (See, Table 9) respectively. The patters of treatment efficacy on LRR and BCSS were most heterogeneous for the varying levels of risk of recurrence scores particularly for patients with higher ROR-C (i.e., intrinsic subtypes centroids and tumor size) (See, Table 9) had poorest prognosis, but may benefit from adjuvant RT.

TABLE 9
Subpopulation treatment effect pattern plot analysis of the treatment effect
of RT versus no RT as measured by 10-year and 20-yr LRR and BCSS.

| | | LRR | | BCSS | |
|---|---|---|---|---|---|
| | Treatment-covariate | (n = 145) | | (n = 145) | |
| Covariate | interaction test | 10-yr | 20-yr | 10-yr | 20-yr |
| RB-1 | KM based p-value | 0.08 | 0.03 | 0.49 | 0.4 |
| | HR based p-value | 0.03 | 0.03 | 0.41 | 0.41 |
| Proliferation Score | KM based p-value | 0.02 | 0.06 | 0.17 | 0.6 |
| | HR based p-value | 0.06 | 0.06 | 0.24 | 0.24 |
| ROR-C | KM based p-value | 0.01 | 0.35 | <0.0001 | 0.06 |
| | HR based p-value | 0.21 | 0.2 | 0.02 | 0.02 |
| ROR-PC | KM based p-value | 0.02 | 0.11 | 0.09 | 0.36 |
| | HR based p-value | 0.1 | 0.09 | 0.04 | 0.06 |

KM = Kaplan-Meier.
HR = Hazard Ratio.

### CONCLUSION

RB1, proliferation score and risk of recurrence signatures predict LRR and BCSS benefit for adjuvant radiation therapy in this study. The clinical utility of these biomarkers as predictors for adjuvant radiation therapy requires confirmation in a second independent trial.

What is claimed is:

1. A method of predicting local-regional relapse free survival or breast cancer specific survival in a subject having breast cancer comprising:
(a) obtaining a biological sample from the subject; and
(b) assaying the biological sample to determine whether the biological sample is classified as a Luminal A, Luminal B, HER2-enriched or Basal-like subtype, wherein the subtype is determined using a measurement of at least 40 of the genes listed in Table 1,
wherein if the biological sample is classified as a Luminal A or Basal-like subtype, a post-mastectomy breast cancer treatment comprising radiation is more likely to prolong local-regional relapse free survival or breast cancer specific survival of the subject and wherein if the biological sample is classified as a Luminal B or HER2-enriched subtype, a post-mastectomy breast cancer treatment comprising radiation is not likely to prolong local-regional relapse free survival or breast cancer specific survival of the subject.

2. A method of screening for the likelihood of the effectiveness of a post-mastectomy breast cancer treatment comprising radiation in a subject in need thereof comprising:
(a) obtaining a biological sample from the subject; and
(b) assaying the biological sample to determine whether the biological sample is classified as a Luminal A, Luminal B, HER2-enriched or Basal-like subtype, wherein the subtype is determined using a measurement of at least 40 of the genes listed in Table 1;
wherein if the biological sample is classified as a Luminal A or Basal-like subtype, a post-mastectomy breast cancer treatment comprising radiation is more likely to be effective in the subject and wherein if the biological sample is classified as a Luminal B or HER2-enriched subtype, the post-mastectomy breast cancer treatment comprising radiation is not likely to be effective in the subject.

3. A method of treating breast cancer in a subject in need thereof comprising:
(a) obtaining a biological sample from the subject;
(b) assaying the biological sample to determine whether the biological sample is classified as a Luminal A, Luminal B, HER2-enriched or Basal-like subtype, wherein the subtype is determined using a measurement of at least 40 of the genes listed in Table 1; and
(c) administering a breast cancer treatment to the subject, wherein if the biological sample is classified as a Luminal A or Basal-like subtype, the subject is administered a post-mastectomy breast cancer treatment comprising radiation and wherein if the biological sample is classified as a Luminal B or HER2-enriched subtype, the subject is administered a breast cancer treatment not comprising radiation, thereby treating breast cancer in the subject.

4. The method of claim 3, wherein assaying includes detecting expression levels of at the least the following 24 genes from the at least 40 of the genes listed in Table 1: FOXA1, MLPH, ESR1, FOXC1, CDC20, ANLN, MAPT, ORC6L, CEP55, MKI67, UBE2C, KNTC2, EXO1, PTTG1, MELK, BIRC5, GPR160, RRM2, SRFP1, NAT1, KIF2C, CXXC5, MIA and BCL2.

5. The method of claim 4, wherein expression levels of at least CCNE1, CDC6, CDCA1, CENPF, TYMS, and UBE2T are additionally detected.

6. The method of claim 4, wherein assaying includes generating a gene expression profile based on said expression of said genes for the biological sample.

7. The method of claim 6, wherein assaying includes comparing the gene expression profile for the biological sample to centroids constructed from gene expression data for the at least 40 of the genes listed in Table 1 for the Luminal A, Luminal B, HER2-enriched or Basal-like subtypes.

8. The method of claim 7, wherein assaying includes utilizing a supervised algorithm and calculating the distance of the gene expression profile for the biological sample to each of the centroids.

9. The method of claim 8, wherein assaying includes classifying the biological sample as a Luminal A, Luminal B, HER2-enriched or Basal-like subtype based upon the nearest centroid.

10. The method of claim 3, wherein assaying includes detecting expression levels of HER2.

11. The method of claim 3, wherein assaying includes detecting expression levels of at least 46 of the genes listed in Table 1.

12. The method of claim 3, wherein assaying includes detecting expression levels of the NANO46 gene set.

13. The method of claim 3, wherein assaying includes detecting expression levels of all 50 genes listed in Table 1.

14. The method of claim 3, wherein the biological sample is selected from the group consisting of a cell, tissue and bodily fluid;
wherein the tissue is obtained from a biopsy and wherein the bodily fluid is selected from the group consisting of blood, lymph, urine, saliva and nipple aspirate.

15. The method of claim 3, wherein the biological sample is an estrogen receptor positive tumor.

16. The method of claim 3, wherein the breast cancer is primary breast cancer.

17. The method of claim 3, wherein the breast cancer is locally advanced or metastatic breast cancer.

18. The method of claim 3, wherein the breast cancer is a node-positive breast cancer.

19. The method of claim 3, wherein if the biological sample is an estrogen receptor positive tumor.

20. The method of claim 3, wherein assaying the biological sample to determine whether the biological sample is classified as a Luminal A, Luminal B, HER2-enriched or Basal-like subtype includes RNA expression profiling, immunohistochemistry (IHC) or fluorescence in situ hybridization (FISH).

21. The method of claim 3, wherein the breast cancer treatment comprising radiation further comprises one or more anti-cancer agents selected from the group consisting of anthracycline agents, alkylating agents, nucleoside analogs, platinum agents, taxanes, vinca agents, anti-estrogen drugs, aromatase inhibitors, ovarian suppression agents, endocrine/hormonal agents, bisphophonate therapy agents and targeted biological therapy agents;
wherein specific anti-cancer or chemotherapeutic agents are selected from the group include cyclophosphamide, fluorouracil (or 5-fluorouracil or 5-FU), methotrexate, thiotepa, carboplatin, cisplatin, gemcitabine, anthracycline, taxanes, paclitaxel, protein-bound paclitaxel, docetaxel, vinorelbine, tamoxifen, raloxifene, toremifene, fulvestrant, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb and bevacizumab, or combinations thereof.

22. The method of claim 21, wherein the anti-cancer agent is cyclophosphamide, fluorouracil (or 5-fluorouracil or 5-FU), methotrexate, or combinations thereof.

23. The method of claim 3, further comprising
determining a proliferation score based on the expression of a subset of proliferation genes in the genes listed in Table 1,
calculating a risk of recurrence (ROR) score using a weighted sum of the classified subtype, proliferation score and optionally one or more clinicopathological variables selected from the group consisting of tumor size, nodal status and histological grade; and
determining whether the subject has a low or high risk of recurrence based on the risk of recurrence score, wherein if the subject has a low risk of recurrence administering a treatment comprising radiation to prolong local-regional relapse free survival or if the subject has a high risk of recurrence administering a treatment comprising radiation to prolong breast cancer specific survival of the subject.

24. The method of claim 23, wherein determining a proliferation signature based on the expression of a subset of proliferation genes in the gene list of Table 1 comprises determining the expression of each of the genes selected from ANLN, CCNEJ, CDC20, CDC6, CDCA1, CENPF, CEP55, EXO1, KIF2C, KNTC2, MELK, MKI67, ORC6L, PTTGJ, RRM2, TYMS, UBE2C and UBE2T.

25. A kit for predicting local-regional relapse free survival or breast cancer specific survival in a subject having breast cancer comprising
reagents sufficient for the detection of at least 40 of the genes listed in Table 1; and
instructions for performing an assay to determine whether a biological sample from said subject is classified as a Luminal A, Luminal B, HER2-enriched or Basal-like subtype, by using said reagents to measure of at least 40 of the genes listed in Table 1, wherein if the biological sample is classified as a Luminal A or Basal-like subtype, a post-mastectomy breast cancer treatment comprising radiation is more likely to prolong local-regional relapse free survival or breast cancer specific survival of the subject and wherein if the biological sample is classified as a Luminal B or HER2-enriched subtype, a post-mastectomy breast cancer treatment comprising radiation is not likely to prolong local-regional relapse free survival or breast cancer specific survival of the subject.

26. A kit for screening for the likelihood of the effectiveness of a post-mastectomy breast cancer treatment comprising radiation in a subject in need thereof comprising
reagents sufficient for the detection of at least 40 of the genes listed in Table 1; and
instructions for performing an assay to determine whether a biological sample from said subject is classified as a Luminal A, Luminal B, HER2-enriched or Basal-like subtype, by using said reagents to measure of at least 40 of the genes listed in Table 1, wherein if the biological sample is classified as a Luminal A or Basal-like subtype, a post-mastectomy breast cancer treatment comprising radiation is more likely to be effective in the subject and wherein if the biological sample is classified as a Luminal B or HER2-enriched subtype, a post-mastectomy breast cancer treatment comprising radiation is not likely to be effective in the subject.

27. A kit for treating breast cancer in a subject in need thereof comprising
reagents sufficient for the detection of at least 40 of the genes listed in Table 1;
instructions for performing an assay to determine whether a biological sample from said subject is classified as a Luminal A, Luminal B, HER2-enriched or Basal-like subtype, by using said reagents to measure of at least 40 of the genes listed in Table 1; and
instructions for administering a post-mastectomy breast cancer treatment comprising radiation if the biological sample is classified as a Luminal A or Basal like subtype and instructions for administering a post-mastectomy breast cancer treatment not comprising radiation if the biological sample is classified as a Luminal B or HER2-enriched subtype.

28. The kit of claim 27, wherein the kit provides reagents sufficient for the detection of at least 46 of the genes listed in Table 1.

29. The kit of claim 27, further comprising
reagents sufficient for the detection of the proliferation genes selected from ANLN, CCNE1, CDC20, CDC6, CDCA1, CENPF, CEP55, EXO1, KIF2C, KNTC2, MELK, MKI67, ORC6L, PTTG1, RRM2, TYMS, UBE2C and UBE2T,
instructions for performing an assay to determine a proliferation score based on the expression of the proliferation genes,
instructions for calculating a risk of recurrence score using a weighted sum of the classified subtype, proliferation score and optionally one or more clinicopathological variables selected from the group consisting of tumor size, nodal status and histological grade; and
instructions for determining whether the subject has a low or high risk of recurrence based on the risk of recurrence score, wherein if the subject has a low risk of recurrence administering a treatment comprising radiation to prolong local-regional relapse free survival or if the subject has a high risk of recurrence administering a treatment comprising radiation to prolong breast cancer specific survival of the subject.


SEQUENCE LISTING (213 sequences)

SEQ ID NO: 1 - DNA, 19 bp, Artificial Sequence
  Synthetic Oligonucleotide
aaagattcctgggacctga

SEQ ID NO: 2 - DNA, 21 bp, Artificial Sequence
  Synthetic Oligonucleotide
acagccactttcagaagcaag

SEQ ID NO: 3 - DNA, 22 bp, Artificial Sequence
  Synthetic Oligonucleotide
ctggaagagttgaataaagagc

SEQ ID NO: 4 - DNA, 18 bp, Artificial Sequence
  Synthetic Oligonucleotide
tacctgaaccggcacctg

SEQ ID NO: 5 - DNA, 20 bp, Artificial Sequence
  Synthetic Oligonucleotide
gcacaaagccattctaagtc

SEQ ID NO: 6 - DNA, 17 bp, Artificial Sequence
  Synthetic Oligonucleotide
gctggctgagcagaaag

SEQ ID NO: 7 - DNA, 19 bp, Artificial Sequence
  Synthetic Oligonucleotide
ctttcgcctgagcctattt

SEQ ID NO: 8 - DNA, 18 bp, Artificial Sequence
  Synthetic Oligonucleotide
ggccaaaatcgacaggac

SEQ ID NO: 9 - DNA, 19 bp, Artificial Sequence
  Synthetic Oligonucleotide
ctgtctgagtgccgtggat

SEQ ID NO: 10 - DNA, 20 bp, Artificial Sequence
  Synthetic Oligonucleotide
gtaaatcaccttctgagcct

SEQ ID NO: 11 - DNA, 18 bp, Artificial Sequence
  Synthetic Oligonucleotide
ggaggcggaagaaaccag

SEQ ID NO: 12 - DNA, 24 bp, Artificial Sequence
  Synthetic Oligonucleotide
gacaaggagaatcaaaagatcagc

SEQ ID NO: 13 - DNA, 17 bp, Artificial Sequence
  Synthetic Oligonucleotide
gtggcagcagatcacaa

SEQ ID NO: 14 - DNA, 20 bp, Artificial Sequence
  Synthetic Oligonucleotide
cctcacgaattgctgaactt

SEQ ID NO: 15 - DNA, 23 bp, Artificial Sequence
  Synthetic Oligonucleotide
catgaaatagtgcatagtttgcc

SEQ ID NO: 16 - DNA, 25 bp, Artificial Sequence
  Synthetic Oligonucleotide
acacagaatctatacccaccagagt

SEQ ID NO: 17 - DNA, 20 bp, Artificial Sequence
  Synthetic Oligonucleotide
gctggctctcacactgatag

SEQ ID NO: 18 - DNA, 18 bp, Artificial Sequence
  Synthetic Oligonucleotide
gcagggagaggagtttgt

SEQ ID NO: 19 - DNA, 23 bp, Artificial Sequence
  Synthetic Oligonucleotide
cccatccatgtgaggaagtataa

SEQ ID NO: 20 - DNA, 17 bp, Artificial Sequence
  Synthetic Oligonucleotide
cttcttggaccttggcg

SEQ ID NO: 21 - DNA, 17 bp, Artificial Sequence
  Synthetic Oligonucleotide
gctactacgcagacacg

SEQ ID NO: 22 - DNA, 19 bp, Artificial Sequence
  Synthetic Oligonucleotide
gatgttcgagtcacagagg

SEQ ID NO: 23 - DNA, 17 bp, Artificial Sequence
  Synthetic Oligonucleotide
ttcggctggaaggaacc

SEQ ID NO: 24 - DNA, 18 bp, Artificial Sequence
  Synthetic Oligonucleotide
cgtggcagatgtgaacga

SEQ ID NO: 25 - DNA, 20 bp, Artificial Sequence
  Synthetic Oligonucleotide
ggagatccgtcaactccaaa

SEQ ID NO: 26 - DNA, 18 bp, Artificial Sequence
  Synthetic Oligonucleotide
tgggtcgtgtcaggaaac

SEQ ID NO: 27 - DNA, 20 bp, Artificial Sequence
  Synthetic Oligonucleotide
cgcagtcatccagagatgtg

SEQ ID NO: 28 - DNA, 21 bp, Artificial Sequence
  Synthetic Oligonucleotide
actcagtacaagaaagaaccg

SEQ ID NO: 29 - DNA, 22 bp, Artificial Sequence
  Synthetic Oligonucleotide
gttggaccagtcaacatctctg

SEQ ID NO: 30 - DNA, 18 bp, Artificial Sequence
  Synthetic Oligonucleotide
tgtggctcattaggcaac

SEQ ID NO: 31 - DNA, 18 bp, Artificial Sequence
  Synthetic Oligonucleotide
gactccaagcgcgaaaac

SEQ ID NO: 32 - DNA, 23 bp, Artificial Sequence
  Synthetic Oligonucleotide
ccacaaaatattcatggttcttg

SEQ ID NO: 33 - DNA, 18 bp, Artificial Sequence
  Synthetic Oligonucleotide
ccagtagcattgtccgag

SEQ ID NO: 34 - DNA, 19 bp, Artificial Sequence
  Synthetic Oligonucleotide
gtctctggtaatgcacact

SEQ ID NO: 35 - DNA, 18 bp, Artificial Sequence
  Synthetic Oligonucleotide
gtggaatgcctgctgacc

SEQ ID NO: 36 - DNA, 18 bp, Artificial Sequence
  Synthetic Oligonucleotide
aggggtgccctctgagat

SEQ ID NO: 37 - DNA, 18 bp, Artificial Sequence
  Synthetic Oligonucleotide
cgagatcgccaagatgtt

SEQ ID NO: 38 - DNA, 18 bp, Artificial Sequence
  Synthetic Oligonucleotide
aggcgaacacacaacgtc

SEQ ID NO: 39 - DNA, 19 bp, Artificial Sequence
  Synthetic Oligonucleotide
agcctcgaacaattgaaga

SEQ ID NO: 40 - DNA, 27 bp, Artificial Sequence
  Synthetic Oligonucleotide
atcgactgtgtaaacaactagagaaga

SEQ ID NO: 41 - DNA, 21 bp, Artificial Sequence
  Synthetic Oligonucleotide
tttaagagggcaaatggaagg

SEQ ID NO: 42 - DNA, 18 bp, Artificial Sequence
  Synthetic Oligonucleotide
tgccgcagaactcacttg

SEQ ID NO: 43 - DNA, 20 bp, Artificial Sequence
  Synthetic Oligonucleotide
cctcagatgatgcctatcca

SEQ ID NO: 44 - DNA, 19 bp, Artificial Sequence
  Synthetic Oligonucleotide
cagcaagcgatggcatagt

SEQ ID NO: 45 - DNA, 17 bp, Artificial Sequence
  Synthetic Oligonucleotide
aatgccaccgaagcctc

SEQ ID NO: 46 - DNA, 23 bp, Artificial Sequence
  Synthetic Oligonucleotide
tcgaactgaaggctatttacgag

SEQ ID NO: 47 - DNA, 18 bp, Artificial Sequence
  Synthetic Oligonucleotide
gtcgaagccgcaattagg

SEQ ID NO: 48 - DNA, 19 bp, Artificial Sequence
  Synthetic Oligonucleotide
caaacgtgtgttctggagg

SEQ ID NO: 49 - DNA, 20 bp, Artificial Sequence
  Synthetic Oligonucleotide
tgccctgtatgatgtcagga

SEQ ID NO: 50 - DNA, 20 bp, Artificial Sequence
  Synthetic Oligonucleotide
gtgaggggtgtcagctcagt

SEQ ID NO: 51 - DNA, 22 bp, Artificial Sequence
  Synthetic Oligonucleotide
tggggcagttctgtattacttc

SEQ ID NO: 52 - DNA, 24 bp, Artificial Sequence
  Synthetic Oligonucleotide
cgatggttttgtacaagatttctc

SEQ ID NO: 53 - DNA, 17 bp, Artificial Sequence
  Synthetic Oligonucleotide
gcaaatccttgggcaga

SEQ ID NO: 54 - DNA, 20 bp, Artificial Sequence
  Synthetic Oligonucleotide
gccgtacagttccacaaagg

SEQ ID NO: 55 - DNA, 22 bp, Artificial Sequence
  Synthetic Oligonucleotide
gacgcttcctatcactctattc

SEQ ID NO: 56 - DNA, 22 bp, Artificial Sequence
  Synthetic Oligonucleotide
ttcctccatcaagagttcaaca

SEQ ID NO: 57 - DNA, 18 bp, Artificial Sequence
  Synthetic Oligonucleotide
gggcacatccagatgttt

SEQ ID NO: 58 - DNA, 19 bp, Artificial Sequence
  Synthetic Oligonucleotide
gggtctgcacagactgcat

SEQ ID NO: 59 - DNA, 20 bp, Artificial Sequence
  Synthetic Oligonucleotide
tccttgtaatggggagacca

SEQ ID NO: 60 - DNA, 23 bp, Artificial Sequence
  Synthetic Oligonucleotide
acttgggatatgtgaataagacc

SEQ ID NO: 61 - DNA, 20 bp, Artificial Sequence
  Synthetic Oligonucleotide
ggggaaagacaaagtttcca

SEQ ID NO: 62 - DNA, 20 bp, Artificial Sequence
  Synthetic Oligonucleotide
actgtctgggtccatggcta

SEQ ID NO: 63 - DNA, 18 bp, Artificial Sequence
  Synthetic Oligonucleotide
ggatttcgtggtgggttc

SEQ ID NO: 64 - DNA, 20 bp, Artificial Sequence
  Synthetic Oligonucleotide
ccacagtctgtgataaacgg

SEQ ID NO: 65 - DNA, 24 bp, Artificial Sequence
  Synthetic Oligonucleotide
ccatcaacattctctttatgaacg

SEQ ID NO: 66 - DNA, 20 bp, Artificial Sequence
  Synthetic Oligonucleotide
atcaactcccaaacggtcac

SEQ ID NO: 67 - DNA, 20 bp, Artificial Sequence
  Synthetic Oligonucleotide
gcccttacacatcggagaac

SEQ ID NO: 68 - DNA, 18 bp, Artificial Sequence
  Synthetic Oligonucleotide
gacttcagggtgctggac

SEQ ID NO: 69 - DNA, 22 bp, Artificial Sequence
  Synthetic Oligonucleotide
tgtgaagccagcaatatgtatc

SEQ ID NO: 70 - DNA, 21 bp, Artificial Sequence
  Synthetic Oligonucleotide
tattgggaggcaggaggttta

SEQ ID NO: 71 - DNA, 20 bp, Artificial Sequence
  Synthetic Oligonucleotide
ctgagttcatgttgctgacc

SEQ ID NO: 72 - DNA, 19 bp, Artificial Sequence
  Synthetic Oligonucleotide
gacagctactattcccgtt

SEQ ID NO: 73 - DNA, 21 bp, Artificial Sequence
  Synthetic Oligonucleotide
tatgtgagtaagctcggagac

SEQ ID NO: 74 - DNA, 17 bp, Artificial Sequence
  Synthetic Oligonucleotide
agtgggcatcccgtaga

SEQ ID NO: 75 - DNA, 19 bp, Artificial Sequence
  Synthetic Oligonucleotide
agtggacatgcgagtggag

SEQ ID NO: 76 - DNA, 18 bp, Artificial Sequence
  Synthetic Oligonucleotide
caccgctggaaactgaac

SEQ ID NO: 77 - DNA, 19 bp, Artificial Sequence
  Synthetic Oligonucleotide
cgtgcacatccatgacctt

SEQ ID NO: 78 - DNA, 17 bp, Artificial Sequence
  Synthetic Oligonucleotide
gaggagatgaccttgcc

SEQ ID NO: 79 - DNA, 18 bp, Artificial Sequence
  Synthetic Oligonucleotide
gccatagccactgccact

SEQ ID NO: 80 - DNA, 17 bp, Artificial Sequence
  Synthetic Oligonucleotide
cttcgactggactctgt

SEQ ID NO: 81 - DNA, 23 bp, Artificial Sequence
  Synthetic Oligonucleotide
cagacatgttggtattgcacatt

SEQ ID NO: 82 - DNA, 20 bp, Artificial Sequence
  Synthetic Oligonucleotide
aggcgatcctgggaaattat

SEQ ID NO: 83 - DNA, 19 bp, Artificial Sequence
  Synthetic Oligonucleotide
cccatttgtctgtcttcac

SEQ ID NO: 84 - DNA, 18 bp, Artificial Sequence
  Synthetic Oligonucleotide
ctgatggttgaggctgtt

SEQ ID NO: 85 - DNA, 19 bp, Artificial Sequence
  Synthetic Oligonucleotide
cgcactccagcacctagac

SEQ ID NO: 86 - DNA, 21 bp, Artificial Sequence
  Synthetic Oligonucleotide
tcacagggtcaaacttccagt

SEQ ID NO: 87 - DNA, 21 bp, Artificial Sequence
  Synthetic Oligonucleotide
gatggtagagttccagtgatt

SEQ ID NO: 88 - DNA, 18 bp, Artificial Sequence
  Synthetic Oligonucleotide
tctggtcacgcagggcaa

SEQ ID NO: 89 - DNA, 20 bp, Artificial Sequence
  Synthetic Oligonucleotide
acacagatgatggagatgtc

SEQ ID NO: 90 - DNA, 25 bp, Artificial Sequence
  Synthetic Oligonucleotide
agtagctacatctccaggttctctg

SEQ ID NO: 91 - DNA, 21 bp, Artificial Sequence
  Synthetic Oligonucleotide
cggattttatcaacgatgcag

SEQ ID NO: 92 - DNA, 19 bp, Artificial Sequence
  Synthetic Oligonucleotide
catttgccgtccttcatcg

SEQ ID NO: 93 - DNA, 20 bp, Artificial Sequence
  Synthetic Oligonucleotide
gcaggtcaaaactctcaaag

SEQ ID NO: 94 - DNA, 20 bp, Artificial Sequence
  Synthetic Oligonucleotide
agcgggcttctgtaatctga

SEQ ID NO: 95 - DNA, 19 bp, Artificial Sequence
  Synthetic Oligonucleotide
gcctcagatttcaactcgt

SEQ ID NO: 96 - DNA, 21 bp, Artificial Sequence
  Synthetic Oligonucleotide
ctgctgagaatcaaagtggga

SEQ ID NO: 97 - DNA, 19 bp, Artificial Sequence
  Synthetic Oligonucleotide
ggaacaaactgctctgcca

SEQ ID NO: 98 - DNA, 22 bp, Artificial Sequence
  Synthetic Oligonucleotide
acagctctttagcatttgtgga

SEQ ID NO: 99 - DNA, 23 bp, Artificial Sequence
  Synthetic Oligonucleotide
gggactatcaatgttgggttctc

SEQ ID NO: 100 - DNA, 21 bp, Artificial Sequence
  Synthetic Oligonucleotide
cacacagttcactgctccaca

SEQ ID NO: 101 - DNA, 100 bp, Artificial Sequence
  Synthetic Oligonucleotide
ccagaagaagtttgttatagacgttggttacgaaagattcctgggacctgaaatattctttcacccggagtttgccaacccagactttatggagtccatc

SEQ ID NO: 102 - DNA, 100 bp, Artificial Sequence
  Synthetic Oligonucleotide
cgtgccaggcgagagaatcttcagagaaaaatggctgagaggcccacagcagctccaaggtctatgactcatgctaagcgagctagacagccactttcag

SEQ ID NO: 103 - DNA, 100 bp, Artificial Sequence
  Synthetic Oligonucleotide
cttcatgttacctcccagcagggcagcagtgaaccagttgtccaagacctggcccaggttgttgaagaggtcataggggttccacagtcttttcagaaac

SEQ ID NO: 104 - DNA, 100 bp, Artificial Sequence
  Synthetic Oligonucleotide
ccaagcaccgcttcgtgtggctccacctggatgttctgtgcctgtaaacatagattcgctttccatgttgttggccggatcaccatctgaagagcagacg

SEQ ID NO: 105 - DNA, 100 bp, Artificial Sequence
  Synthetic Oligonucleotide
ttcctgaaaaaagaagtggtggggaaagacctgctgaaagggtcgctcctcttcacagctggcccgttggaagaagagcggtttggcttccctgcattca

SEQ ID NO: 106 - DNA, 100 bp, Artificial Sequence
  Synthetic Oligonucleotide
gagaactgtgtcaagtggatggttccatttgccatggttataagggagacggggagctcaaaactgaagcacttcaggggcgtcgctgatgaagatgcac

SEQ ID NO: 107 - DNA, 100 bp, Artificial Sequence
  Synthetic Oligonucleotide
cccgagtgggctccctaagctggaacagctatatcctgtccagtggttcacgttctggccacatccaccaccatgatgttcgggtagcagaacaccatgt

SEQ ID NO: 108 - DNA, 100 bp, Artificial Sequence
  Synthetic Oligonucleotide
ggggaagttatatgaagcctacagtaaagtctgtcgcaaacagcaggtggcggctgtggaccagtcagagtgtttgtcactttcagggctcttggaagcc

SEQ ID NO: 109 - DNA, 100 bp, Artificial Sequence
  Synthetic Oligonucleotide
gcctggcggtgttttcgtcgtgctcagcggtgggaggaggcggaagaaaccagagcctgggagattaacaggaaacttccaagatggaaactttgtcttt

SEQ ID NO: 110 - DNA, 100 bp, Artificial Sequence
  Synthetic Oligonucleotide
ccctcgaccgtgaggatgagcagtttgtgaggaacaacatctatgaagtcatggtcttggccatggacaatggaagccctcccaccactggcacgggaac

SEQ ID NO: 111 - DNA, 100 bp, Artificial Sequence
  Synthetic Oligonucleotide
agaaaatcttgcagagtcctccaaaccaacagctggtggcagcagatcacaaaaggtcaaagttgctcagcggagcccagtagattcaggcaccatcctc

SEQ ID NO: 112 - DNA, 100 bp, Artificial Sequence
  Synthetic Oligonucleotide
gtactaccgcattgcttgaacagctggaagagacaacgagagaaggagaaaggagggagcaggtgttgaaagccttatctgaagagaaagacgtattgaa

SEQ ID NO: 113 - DNA, 100 bp, Artificial Sequence
  Synthetic Oligonucleotide
agctgccctctccgtgcaatgtcactgctcgtgtggtctccagcaagggattcgggcgaagacaaacggatgcacccgtctttagaaccaaaaatattct

SEQ ID NO: 114 - DNA, 100 bp, Artificial Sequence
  Synthetic Oligonucleotide
gcagccaggaacgtactggtgaaaacaccgcagcatgtcaagatcacagattttgggctggccaaactgctgggtgcggaagagaaagaataccatgcag

SEQ ID NO: 115 - DNA, 100 bp, Artificial Sequence
  Synthetic Oligonucleotide
tgaaggtgcttggatctggcgcttttggcacagtctacaagggcatctggatccctgatggggagaatgtgaaaattccagtggccatcaaagtgttgag

SEQ ID NO: 116 - DNA, 100 bp, Artificial Sequence
  Synthetic Oligonucleotide
aggaaccagggaaaatgtgtagagggcatggtggagatcttcgacatgctgctggctacatcatctcggttccgcatgatgaatctgcagggagaggagt

SEQ ID NO: 117 - DNA, 100 bp, Artificial Sequence
  Synthetic Oligonucleotide
tggcccacaaagtaattaaagctgcccggtctcagggggtagattgcctcgtggctccctatgaagctgatgcgcagttggcctatcttaacaaagcggg

SEQ ID NO: 118 - DNA, 100 bp, Artificial Sequence
  Synthetic Oligonucleotide
cccacatccagtggctgaagcacatcgtcatcaacggcagcagcttcggagccgacggtttcccctatgtgcaagtcctaaagactgcagacatcaatag

SEQ ID NO: 119 - DNA, 100 bp, Artificial Sequence
  Synthetic Oligonucleotide
tggatggttgtattgggcagggtggctccaggatgttaggaactgtgaagatggaagggcatgaaaccagcgactggaacagctactacgcagacacgca

SEQ ID NO: 120 - DNA, 100 bp, Artificial Sequence
  Synthetic Oligonucleotide
ttcgagtcacagaggatcggcttgaacaactctccagtgaacgggaatagtagctgtcaaatggccttcccttccagccagtctctgtaccgcacgtccg

SEQ ID NO: 121 - DNA, 100 bp, Artificial Sequence
  Synthetic Oligonucleotide
ggatttcagtccttgcttatgttttgggagacccagccatctaccaaagcctgaaggcacagaatgcttattctcgtcactgtcctttctatgtcagcat

SEQ ID NO: 122 - DNA, 100 bp, Artificial Sequence
  Synthetic Oligonucleotide
gtgtcagctcagtgcatcccaggcagctcttagtgtggagcagtgaactgtgtgtggttccttctacttggggatcatgcagagagcttcacgtctgaag

SEQ ID NO: 123 - DNA, 100 bp, Artificial Sequence
  Synthetic Oligonucleotide
gttgtctacaggttcacagcaaggccactggtacagacaatctttgaaggtggaaaagcaacttgttttgcatatggccagacaggaagtggcaagacac

SEQ ID NO: 124 - DNA, 100 bp, Artificial Sequence
  Synthetic Oligonucleotide
aaaaggtcataagcatgaagcgcagttcagtttccagcggtggtgctggccgcctctccatgcaggagttaagatcccaggatgtaaataaacaaggcct

SEQ ID NO: 125 - DNA, 100 bp, Artificial Sequence
  Synthetic Oligonucleotide
gcagtcatccagagatgtgacctcctccagccgccaaatccgcaccaaggtcatggatgtgcacgatggcaaggtggtgtccacccacgagcaggtcctt

SEQ ID NO: 126 - DNA, 100 bp, Artificial Sequence
  Synthetic Oligonucleotide
ctgactcagtacaagaaagaaccggtgaccacccgtcaggtgcgtaccattgtggaagaggtccaggatggcaaggtcatctcctcccgcgagcaggtcc

SEQ ID NO: 127 - DNA, 100 bp, Artificial Sequence
  Synthetic Oligonucleotide
ctggttctcttgctccaccaggaacaagccaccatgtctcgccagtcaagtgtgtccttccggagcgggggcagtcgtagcttcagcaccgcctctgcca

SEQ ID NO: 128 - DNA, 100 bp, Artificial Sequence
  Synthetic Oligonucleotide
gccgggtccctcaactcaaagctcgcatggtcagtaaaagcaaagacgggactggaagcgatgacaaaaaagccaagacatccacacgttcctctgctaa

SEQ ID NO: 129 - DNA, 100 bp, Artificial Sequence
  Synthetic Oligonucleotide
ggtgaggagcaggcaaatgtgcaataccaacatgtctgtacctactgatggtgctgtaaccacctcacagattccagcttcggaacaagagaccctggtt

SEQ ID NO: 130 - DNA, 100 bp, Artificial Sequence
  Synthetic Oligonucleotide
agagacagccaacaaaatattcatggttcttgagtactgccctggaggagagctgtttgactatataatttcccaggatcgcctgtcagaagaggagacc

SEQ ID NO: 131 - DNA, 100 bp, Artificial Sequence
  Synthetic Oligonucleotide
ccggggccaagtggtgtatgtcttctccaagctgaagggccgtgggcggctcttctggggaggcagcgttcagggagattactatggagatctggctgct

SEQ ID NO: 132 - DNA, 100 bp, Artificial Sequence
  Synthetic Oligonucleotide
gcttccagcagcaaatctcagacagaggttcctaagagaggaggagaaagagtggcaacctgccttcaaaagagagtgtctatcagccgaagtcaacatg

SEQ ID NO: 133 - DNA, 100 bp, Artificial Sequence
  Synthetic Oligonucleotide
gaggaagtcaaacctcccgatatttctccctcgagtggctgggaaacttggcaagagaccagaggacccaaatgcagacccttcaagtgaggccaaggca

SEQ ID NO: 134 - DNA, 100 bp, Artificial Sequence
  Synthetic Oligonucleotide
agcagccaaggccctgatgtccgccttctacacctttcgctacccactgagtctcagcccagatgactgcaggggcgttcaacacctatatggccagccc

SEQ ID NO: 135 - DNA, 100 bp, Artificial Sequence
  Synthetic Oligonucleotide
caccgaggagaatgtcaagaggcgaacacacaacgtcttggagcgccagaggaggaacgagctaaaacggagcttttttgccctgcgtgaccagatcccg

SEQ ID NO: 136 - DNA, 100 bp, Artificial Sequence
  Synthetic Oligonucleotide
agcacttcctcatagaccttggatgtgggaggattgcattcagtctagttcctggttgccggctgaaataacctgaattcaagccaggaagaagcagcaa

SEQ ID NO: 137 - DNA, 100 bp, Artificial Sequence
  Synthetic Oligonucleotide
gactgtgtaaacaactagagaagattggacagcaggtcgacagagaacctggagatgtagctactccaccacggaagagaaagaagatagtggttgaagc

SEQ ID NO: 138 - DNA, 100 bp, Artificial Sequence
  Synthetic Oligonucleotide
gggatgaagcatcaggctgtcattatggtgtccttacctgtgggagctgtaaggtcttctttaagagggcaatggaagggcagcacaactacttatgtgc

SEQ ID NO: 139 - DNA, 100 bp, Artificial Sequence
  Synthetic Oligonucleotide
gcgacggcttcgatgaaggacggcaaatgggagcggaagaagttcatgggaacagagctgaatggaaagaccctgggaattcttggcctgggcaggattg

SEQ ID NO: 140 - DNA, 100 bp, Artificial Sequence
  Synthetic Oligonucleotide
caccagccttacctaaagctactagaaaggctttgggaactgtcaacagagctacagaaaagtctgtaaagaccaagggacccctcaaacaaaaacagcc

SEQ ID NO: 141 - DNA, 100 bp, Artificial Sequence
  Synthetic Oligonucleotide
ttccttttggaccgccgaggaggttgacctctccaaggacattcagcactgggaatccctgaaacccgaggagagatattttatatcccatgttctggct

SEQ ID NO: 142 - DNA, 100 bp, Artificial Sequence
  Synthetic Oligonucleotide
gtgggtcacacacacgcactgcgcctgtcagtagtggacattgtaatccagtcggcttgttcttgcagcattcccgctcccttccctccatagccacgct

SEQ ID NO: 143 - DNA, 100 bp, Artificial Sequence
  Synthetic Oligonucleotide
gatcgaactgaaggctatttacgagcagactcacaagagccctcccactttgattctcagcagcctgcagtcttggaagaagaagaggtcatgatagctc

SEQ ID NO: 144 - DNA, 100 bp, Artificial Sequence
  Synthetic Oligonucleotide
ctggctgccctcagcattgtggccgtcaactattctcttgtttactgccttttgactcggatgaagagacacggaaggggagaaatcattggaattcaga

SEQ ID NO: 145 - DNA, 100 bp, Artificial Sequence
  Synthetic Oligonucleotide
tgctaaagagctgtcttccaagggagtgaaaatctgggatgccaatggatcccgagactttttggacagcctgggattctccaccagagaagaaggggac

SEQ ID NO: 146 - DNA, 100 bp, Artificial Sequence
  Synthetic Oligonucleotide
gtctgccctgtatgatgtcaggaccattctgctctccatccagagccttctaggagaacccaacattgatagtcccttgaacacacatgctgccgagctc

SEQ ID NO: 147 - DNA, 2199 bp, Homo sapiens
cagcggcgctgcggcggctcgcgggagacgctgcgcgcggggctagcgggcggcggagcggacggcgacggggcgctctcgggctgccggcggggccgagcgccgcgcgtcccgagcatggcaggctccctgcctccctgcgtggtggactgtggcaccgggtataccaagcttggctacgcaggcaacactgagccccagttcattattccttcatgtattgccatcagagagtcagcaaaggtagttgaccaagctcaaaggagagtgttgaggggagttgatgaccttgactttttcataggagatgaagccatcgataaacctacatatgctacaaagtggccgatacgacatggaatcattgaagactgggatcttatggaaaggttcatggagcaagtggtttttaaatatcttcgagctgaacctgaggaccattattttttaatgacagaacctccactcaatacaccagaaaacagagagtatcttgcagaaattatgtttgaatcatttaacgtaccaggactctacattgcagttcaggcagtgctggccttggcggcatcttggacatctcgacaagtgggtgaacgtacgttaacggggatagtcattgacagcggagatggagtcacccatgttatcccagtggcagaaggttatgtaattggaagctgcatcaaacacatcccgattgcaggtagagatattacgtatttcattcaacagctgctaagggagagggaggtgggaatccctcctgagcagtcactggagaccgcaaaagccattaaggagaaatactgttacatttgccccgatatagtcaaggaatttgccaagtatgatgtggatccccggaagtggatcaaacagtacacgggtatcaatgcgatcaaccagaagaagtttgttatagacgttggttacgaaagattcctgggacctgaaatattctttcacccggagtttgccaacccagactttatggagtccatctcagatgttgttgatgaagtaatacagaactgccccatcgatgtgcggcgcccgctgtataagaatgtcgtactctcaggaggctccaccatgttcagggatttcggacgccgactgcagagggatttgaagagagtggtggatgctaggctgaggctcagcgaggagctcagcggcgggaggatcaagccgaagcctgtggaggtccaggtggtcacgcatcacatgcagcgctacgccgtgtggttcggaggctccatgctggcctcgactcccgagttctttcaggtctgccacaccaagaaggactatgaagagtacgggcccagcatctgccgccacaaccccgtctttggagtcatgtcctagtgtctgcctgaacgcgtcgttcgatggtgtcacgttggggaacaagtgtccttcagaacccagagaaggccgccgttctgtaaatagcgacgtcggtgttgctgcccagcagcgtgcttgcattgccggtgcatgaggcgcggcgcgggcccttcagtaaaagccatttatccgtgtgccgaccgctgtctgccagcctcctccttctcccgccctcctcaccctcgctctccctcctcctcctcctccgagctgctagctgacaaatacaattctgaaggaatccaaatgtgactttgaaaattgttagagaaaacaacattagaaaatggcgcaaaatcgttaggtcccaggagagaatgtgggggcgcaaacccttttcctcccagcctatttttgtaaataaaatgtttaaacttgaaatacaaatcgatgtttatatttcctatcattttgtattttatggtatttggtacaactggctgatactaagcacgaatagatattgatgttatggagtgctgtaatccaaagtttttaattgtgaggcatgttctgatatgtttataggcaaacaaataaaacagcaaacttttttgccacatgtttgctagaaaatgattatactttattggagtgacatgaagtttgaacactaaacagtaatgtatgagaattactacagatacatgtatcttttagttttttttgtttgaactttctggagctgttttatagaagatgatggtttgttgtcggtgagtgttggatgaaatacttccttgcaccattgtaataaaagctgttagaatatttgtaaatatc

SEQ ID NO: 148 - DNA, 1989 bp, Homo sapiens
cagcggcgctgcggcggctcgcgggagacgctgcgcgcggggctagcgggcggcggagcggacggcgacggggcgctctcgggctgccggcggggccgagcgccgcgcgtcccgagcatggcaggctccctgcctccctgcgtggtggactgtggcaccgggtataccaagcttggctacgcaggcaacactgagccccagttcattattccttcatgtattgccatcagagagtcagcaaaggtagttgaccaagctcaaaggagagtgttgaggggagttgatgaccttgactttttcataggagatgaagccatcgataaacctacatatgctacaaagtggccgatacgacatggaatcattgaagactgggatcttatggaaaggttcatggagcaagtggtttttaaatatcttcgagctgaacctgaggaccattattttttaatgacagaacctccactcaatacaccagaaaacagagagtatcttgcagaaattatgtttgaatcatttaacgtaccaggactctacattgcagttcaggcagtgctggccttggcggcatcttggacatctcgacaagtgggtgaacgtacgttaacggggatagtcattgacagcggagatggagtcacccatgttatcccagtggcagaaggttatgtaattggaagctgcatcaaacacatcccgattgcaggtagagatattacgtatttcattcaacagctgctaagggagagggaggtgggaatccctcctgagcagtcactggagaccgcaaaagccattaaggagaaatactgttacatttgccccgatatagtcaaggaatttgccaagtatgatgtggatccccggaagtggatcaaacagtacacgggtatcaatgcgatcaaccagaagaagtttgttatagacgttggttacgaaagattcctgggacctgaaatattctttcacccggagtttgccaacccagactttatggagtccatctcagatgttgttgatgaagtaatacagaactgccccatcgatgtgcggcgcccgctgtataagcccgagttctttcaggtctgccacaccaagaaggactatgaagagtacgggcccagcatctgccgccacaaccccgtctttggagtcatgtcctagtgtctgcctgaacgcgtcgttcgatggtgtcacgttggggaacaagtgtccttcagaacccagagaaggccgccgttctgtaaatagcgacgtcggtgttgctgcccagcagcgtgcttgcattgccggtgcatgaggcgcggcgcgggcccttcagtaaaagccatttatccgtgtgccgaccgctgtctgccagcctcctccttctcccgccctcctcaccctcgctctccctcctcctcctcctccgagctgctagctgacaaatacaattctgaaggaatccaaatgtgactttgaaaattgttagagaaaacaacattagaaaatggcgcaaaatcgttaggtcccaggagagaatgtgggggcgcaaacccttttcctcccagcctatttttgtaaataaaatgtttaaacttgaaatacaaatcgatgtttatatttcctatcattttgtattttatggtatttggtacaactggctgatactaagcacgaatagatattgatgttatggagtgctgtaatccaaagtttttaattgtgaggcatgttctgatatgtttataggcaaacaaataaaacagcaaacttttttgccacatgtttgctagaaaatgattatactttattggagtgacatgaagtttgaacactaaacagtaatgtatgagaattactacagatacatgtatcttttagttttttttgtttgaactttctggagctgttttatagaagatgatggtttgttgtcggtgagtgttggatgaaatacttccttgcaccattgtaataaaagctgttagaatatttgtaaatatc

SEQ ID NO: 149 - DNA, 4786 bp, Homo sapiens
ctcggcgctgaaattcaaatttgaacggctgcagaggccgagtccgtcactggaagccgagaggagaggacagctggttgtgggagagttcccccgcctcagactcctggttttttccaggagacacactgagctgagactcacttttctcttcctgaatttgaaccaccgtttccatcgtctcgtagtccgacgcctggggcgatggatccgtttacggagaaactgctggagcgaacccgtgccaggcgagagaatcttcagagaaaaatggctgagaggcccacagcagctccaaggtctatgactcatgctaagcgagctagacagccactttcagaagcaagtaaccagcagcccctctctggtggtgaagagaaatcttgtacaaaaccatcgccatcaaaaaaacgctgttctgacaacactgaagtagaagtttctaacttggaaaataaacaaccagttgagtcgacatctgcaaaatcttgttctccaagtcctgtgtctcctcaggtgcagccacaagcagcagataccatcagtgattctgttgctgtcccggcatcactgctgggcatgaggagagggctgaactcaagattggaagcaactgcagcctcctcagttaaaacacgtatgcaaaaacttgcagagcaacggcgccgttgggataatgatgatatgacagatgacattcctgaaagctcactcttctcaccaatgccatcagaggaaaaggctgcttcccctcccagacctctgctttcaaatgcctcggcaactccagttggcagaaggggccgtctggccaatcttgctgcaactatttgctcctgggaagatgatgtaaatcactcatttgcaaaacaaaacagtgtacaagaacagcctggtaccgcttgtttatccaaattttcctctgcaagtggagcatctgctaggatcaatagcagcagtgttaagcaggaagctacattctgttcccaaagggatggcgatgcctctttgaataaagccctatcctcaagtgctgatgatgcgtctttggttaatgcctcaatttccagctctgtgaaagctacttctccagtgaaatctactacatctatcactgatgctaaaagttgtgagggacaaaatcctgagctacttccaaaaactcctattagtcctctgaaaacgggggtatcgaaaccaattgtgaagtcaactttatcccagacagttccatccaagggagaattaagtagagaaatttgtctgcaatctcaatctaaagacaaatctacgacaccaggaggaacaggaattaagcctttcctggaacgctttggagagcgttgtcaagaacatagcaaagaaagtccagctcgtagcacaccccacagaacccccattattactccaaatacaaaggccatccaagaaagattattcaagcaagacacatcttcatctactacccatttagcacaacagctcaagcaggaacgtcaaaaagaactagcatgtcttcgtggccgatttgacaagggcaatatatggagtgcagaaaaaggcggaaactcaaaaagcaaacaactagaaaccaaacaggaaactcactgtcagagcactcccctcaaaaaacaccaaggtgtttcaaaaactcagtcacttccagtaacagaaaaggtgaccgaaaaccagataccagccaaaaattctagtacagaacctaaaggtttcactgaatgcgaaatgacgaaatctagccctttgaaaataacattgtttttagaagaggacaaatccttaaaagtaacatcagacccaaaggttgagcagaaaattgaagtgatacgtgaaattgagatgagtgtggatgatgatgatatcaatagttcgaaagtaattaatgacctcttcagtgatgtcctagaggaaggtgaactagatatggagaagagccaagaggagatggatcaagcattagcagaaagcagcgaagaacaggaagatgcactgaatatctcctcaatgtctttacttgcaccattggcacaaacagttggtgtggtaagtccagagagtttagtgtccacacctagactggaattgaaagacaccagcagaagtgatgaaagtccaaaaccaggaaaattccaaagaactcgtgtccctcgagctgaatctggtgatagccttggttctgaagatcgtgatcttctttacagcattgatgcatatagatctcaaagattcaaagaaacagaacgtccatcaataaagcaggtgattgttcggaaggaagatgttacttcaaaactggatgaaaaaaataatgcctttccttgtcaagttaatatcaaacagaaaatgcaggaactcaataacgaaataaatatgcaacagacagtgatctatcaagctagccaggctcttaactgctgtgttgatgaagaacatggaaaagggtccctagaagaagctgaagcagaaagacttcttctaattgcaactgggaagagaacacttttgattgatgaattgaataaattgaagaacgaaggacctcagaggaagaataaggctagtccccaaagtgaatttatgccatccaaaggatcagttactttgtcagaaatccgcttgcctctaaaagcagattttgtctgcagtacggttcagaaaccagatgcagcaaattactattacttaattatactaaaagcaggagctgaaaatatggtagccacaccattagcaagtacttcaaactctcttaacggtgatgctctgacattcactactacatttactctgcaagatgtatccaatgactttgaaataaatattgaagtttacagcttggtgcaaaagaaagatccctcaggccttgataagaagaaaaaaacatccaagtccaaggctattactccaaagcgactcctcacatctataaccacaaaaagcaacattcattcttcagtcatggccagtccaggaggtcttagtgctgtgcgaaccagcaacttcgcccttgttggatcttacacattatcattgtcttcagtaggaaatactaagtttgttctggacaaggtcccctttttatcttctttggaaggtcatatttatttaaaaataaaatgtcaagtgaattccagtgttgaagaaagaggttttctaaccatatttgaagatgttagtggttttggtgcctggcatcgaagatggtgtgttctttctggaaactgtatatcttattggacttatccagatgatgagaaacgcaagaatcccataggaaggataaatctggctaattgtaccagtcgtcagatagaaccagccaacagagaattttgtgcaagacgcaacacttttgaattaattactgtccgaccacaaagagaagatgaccgagagactcttgtcagccaatgcagggacacactctgtgttaccaagaactggctgtctgcagatactaaagaagagcgggatctctggatgcaaaaactcaatcaagttcttgttgatattcgcctctggcaacctgatgcttgctacaaacctattggaaagccttaaaccgggaaatttccatgctatctagaggtttttgatgtcatcttaagaaacacacttaagagcatcagatttactgattgcattttatgctttaagtacgaaagggtttgtgccaatattcactacgtattatgcagtatttatatcttttgtatgtaaaactttaactgatttctgtcattcatcaatgagtagaagtaaatacattatagttgattttgctaaatcttaatttaaaagcctcattttcctagaaatctaattattcagttattcatgacaatatttttttaaaagtaagaaattctgagttgtcttcttggagctgtaggtcttgaagcagcaacgtctttcaggggttggagacagaaacccattctccaatctcagtagttttttcgaaaggctgtgatcatttattgatcgtgatatgacttgttactagggtactgaaaaaaatgtctaaggcctttacagaaacatttttagtaatgaggatgagaactttttcaaatagcaaatatatattggcttaaagcatgaggctgtcttcagaaaagtgatgtggacataggaggcaatgtgtgagacttgggggttcaatattttatatagaagagttaataagcacatggtttacatttactcagctactatatatgcagtgtggtgcacattttcacagaattctggcttcattaagatcattatttttgctgcgtagcttacagacttagcatattagttttttctactcctacaagtgtaaattgaaaaatctttatattaaaaaagtaaactgttatgaagctgctatgtactaataatactttgcttgccaaagtgtttgggttttgttgttgtttgtttgtttgtttgtttttggttcatgaacaacagtgtctagaaacccattttgaaagtggaaaattattaagtcacctatcacctttaaacgcctttttttaaaattataaaatattgtaaagcagggtctcaacttttaaatacactttgaacttcttctctgaattattaaagttctttatgacctcatttataaacactaaattctgtcacctcctgtcattttattttttattcattcaaatgtattttttcttgtgcatattataaaaatatattttatgagctcttactcaaataaatacctgtaaatgtctaaaggaaaaaaaaaaaaaaaaaa

SEQ ID NO: 150 - DNA, 3885 bp, Homo sapiens
aggccggggcggggctgggaagtagtcgggcggggttgtgagacgccgcgctcagcttccatcgctgggcggtcaacaagtgcgggcctggctcagcgcgggggggcgcggagaccgcgaggcgaccgggagcggctgggttcccggctgcgcgcccttcggccaggccgggagccgcgccagtcggagcccccggcccagcgtggtccgcctccctctcggcgtccacctgcccggagtactgccagcgggcatgaccgacccaccaggggcgccgccgccggcgctcgcaggccgcggatgaagaagaaaacccggcgccgctcgacccggagcgaggagttgacccggagcgaggagttgaccctgagtgaggaagcgacctggagtgaagaggcgacccagagtgaggaggcgacccagggcgaagagatgaatcggagccaggaggtgacccgggacgaggagtcgacccggagcgaggaggtgaccagggaggaaatggcggcagctgggctcaccgtgactgtcacccacagcaatgagaagcacgaccttcatgttacctcccagcagggcagcagtgaaccagttgtccaagacctggcccaggttgttgaagaggtcataggggttccacagtcttttcagaaactcatatttaagggaaaatctctgaaggaaatggaaacaccgttgtcagcacttggaatacaagatggttgccgggtcatgttaattgggaaaaagaacagtccacaggaagaggttgaactaaagaagttgaaacatttggagaagtctgtggagaagatagctgaccagctggaagagttgaataaagagcttactggaatccagcagggttttctgcccaaggatttgcaagctgaagctctctgcaaacttgataggagagtaaaagccacaatagagcagtttatgaagatcttggaggagattgacacactgatcctgccagaaaatttcaaagacagtagattgaaaaggaaaggcttggtaaaaaaggttcaggcattcctagccgagtgtgacacagtggagcagaacatctgccaggagactgagcggctgcagtctacaaactttgccctggccgagtgaggtgtagcagaaaaaggctgtgctgccctgaagaatggcgccaccagctctgccgtctctggagcggaatttacctgatttcttcagggctgctgggggcaactggccatttgccaattttcctactctcacactggttctcaatgaaaaatagtgtctttgtgattttgagtaaagctcctatctgttttctccttctgtctctgtggttgtactgtccagcaatccaccttttctggagagggccacctctgcccaaattttcccagctgtttggacctctgggtgctttctttgggctggtgagagctctaatttgccttgggccagtttcaggtttataggccccctcagtcttcagatacatgagggcttctttgctcttgtgatcgtgtagtcccatagctgtaaaaccagaatcaccaggaggttgcacctagtcaggaatattgggaatggcctagaacaaggtgtttggcacataagtagaccacttatccctcattgtgacctaattccagagcatctggctgggttgttgggttctagactttgtcctcacctcccagtgaccctgactagccacaggccatgagataccagggggccgttccttggatggagcctgtggttgatgcaaggcttccttgtccccaagcaagtcttcagaaggttagaacccagtgttgactgagtctgtgcttgaaaccaggccagagccatggattaggaagggcaaagagaaggcaccagaatgagtaaagcaggcaggtggtgaagccaaccataaacttctcaggagtgacatgtgcttccttcaaaggcatttttgttaaccatatccttctgagttctatgtttccttcacagctgttctatccattttgtggactgtcccccacccccaccccatcattgtttttaaaaaattaaggcctggcgcagcagctcatgcctataatcccagcactttgggaggctgaggcgggcggatcacttgaggccaggagtttgagaccagcccaggcaacatagcaaaaccccattctgctttaaaaaaaaaaaaaaaaaaaattagcttggcgtagtggcatgtgcctataatcccagctactggggaggctgaggcacaagaatcatttgaacctgggaggtagaggttgctgtgagccgagattacgcccctgcactccagcctgggtcacagagtgagactccatctcagaaaaaaaaaaaattgagtcaggtgcagtagctccttcctgtagtcccagctacttgggaggctgaggctagaggatcacttgagcccaggagtttgagtctagtctgggcaacatagcaagaccccatctctaaaatttaagtaagtaaaagtagataaataaaaagaaaaaaaaactgtttatgtgctcatcataaagtagaagagtggtttgcttttttttttttttttggattaatgaggaaatcattctgtggctctagtcataatttatgcttaataacattgatagtagccctttgcgctataactctacctaaagactcacatcatttggcagagagagagtcgttgaagtcccaggaattcaggactgggcaggttaagacctcagacaaggtagtagaggtagacttgtggacaaggctcgggtcccagcccaccgcaccccaactttaatcagagtggttcactattgatctatttttgtgtgatagctgtgtggcgtgggccacaacatttaatgagaagttactgtgcaccaaactgccgaacaccattctaaactattcatatatattagtcatttaattcttacataacttgagaggtagacagatatccttattttagagatgaggaaaccaagagaacttaggtcattagcgcaaggttgtagagtaagcggcaaagccaagacacaaagctgggtggtttggtttcagagccagtgcttttcccctctactgtactgcctctcaaccaacacagggttgcacaggcccattctctgatttttttcctcttgtcctctgcctctccctctagctcccacttcctctctgctctagttcattttctttagagcagcccgagtgatcatgaagtgcaaatcttgccatgtcagtcccctgcttagaaccctccaatggctcactttctctttaggcaaaagtctttaccccatgccttctcccatctcatctcaaccccctcatttgttggctgtctgctgtcagccactcttctttcaggtcctcagatgcactgcaccctctcctgcctgggggtctttgctcctgctactacctctgcttgaacagctcctcaccttccttcctccaaccctacccttgtataggtgacttttgttcatccttcagaattcaactcacatgtctcttgcatggagaaccctcacctactgtgttgagaccctgtccagcccccaggtgggatcctctctcgacttcccatacatttctttcacagcatttacatagtccatgatagtttacttgtgggattatttggttaatctttgcctttaacaccagggttccttgggtgaaggagcttctttatcttggtaacagcattatttcaagcataacttgtaatatagttatattacatatataacatatatatatataacataacatatataacatatataacaagcataacttgttatatagtcttgtatatagtaagacctcaataaatatttggagaacaaaaaaaaaaaaaaa

SEQ ID NO: 151 - DNA, 6492 bp, Homo sapiens
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 152 - DNA, 2724 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccattgggccgggcacggtggcttacgcctgtaataccagcactttgggaggccgaggcgggcggatcacgagagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 153 - DNA, 4934 bp, Homo sapiens
aatgagggtatttataaactacttaaattataaaaagaatgagacatcagacttacagttttggatactaatttttttcacttaacgttcattatgtgataggagttttccatcctattataccgctgtgcgatctgatcttgggcacgttaaccaacctcttgttgcctcgattttctcacctgtaaaagtgggggtaatcataatgcttacttagtaggatagccctgaagaataagtgacttagcgaacataaatagcttacaatagggttttcagcatgggaaggattcagtaaatgttagctgtcatcatcaccacctacaaaggaagcaatactgtgctgaaagtttttccatcattaatgtaatttctatagtacgattcccaagaagatattaaaattatggaaataaaggtattggtatattcctaattatttcctaaaagattgtattgataaatatgctcatccttcccttaacgggatgcattccagaaaaacaagtcaaatgttagacaaagtatcagaagggaaattctgtagccagagagctaaaaattacaatagggtctctaattatacttcaacttttttaggaataattctcagtgtgttttcccacatttcatatgtaatttttttttttttttttttttgagacagagcctcgccctgtcaccaggctggagtacagtggcgcgatctcggctcactgcaacttccacctgctgggttcaagcaattcttctgacctcaggtgatccacccgcctcggcctcccaaagtgctgggattataacaggcgtggcatgagtcaccgcgcccggccgatctttacttttttattctttgtaccccctgcctatccagttagcatgtgattaaagtcaaagatttgccactttgggccacatctattaattttcatctttgttataattgtatttagtttttgatctacactgcttattactcccagtcattttttatagaactgaaaatctggtaaaatactcaaaattgcactgacttctatgtagaggcgacactccatcagaaccgtgggctgacagggaatcccactgtgcaggagctgcgcgcattttcatttctgattctctttggcgtatccaggactctgatgacatgatcatatatttatcagtagtaacaggttgggccatttgttttttgtggtaaatcatatatttaagattttagaaataagttgatagccatgtattttggaatttgaaaaagacattgcattactcagcttcaaattaagctttaatcaaatagtgaaactttccattaatggacagtgtatacctttttgtgtatttaaaaaaaaaaacactgaatatagtgcctttgtgacaggggagcttggttcctgacaatgtcctcttgagccttttttttttttttgagatggagtctcactgtgtcacccaggctggagtgcagtggcgccatcttggctcactgcaacctccgccccctgggttcaagtgattctcattcctcagcttcctaagtagctgggattacaggcacgcaccaccatgaccagctaatttttatacttttagtagagacagggttttgccatgttggctaggttggtctcgaactcctgacctcaagtaatccacccaccatggcctccccaaagtgctgggattacaggcgtgagccatttcacccggcctctcttccgtctttgagctgtgaggaaatagctacattacatgagctgctagatctgccttatggtcagaaatgaaggttgaactctcaggaacagtgacatatatacacactgatatttccaaagtacaatgccccaaattgatccacaaaggaattaaggtcatttgcaacaaaatcacagaatagtaacaaataaatagaagataaatatggccagggatgctgcaaactgatatactgccaagtttatcagttgggaatcccaacagtgaaaagcataaaaatgaaaggaattttaaggagactttttatagaagagtgggaaggattggaggagccaacaagtgatggtgaggcacacagggaagagcttcagtgggcaccatcccctctctggtttgaaggggtagggaggggaccagagctgggaggagggggctggaatactgctggaggagccactcccttccagacctgctgtggccatcacagaatgcagccactgccagagcagcagcccgaggaaccaggcagggggagcacaagtaccctagcctctctctttctgtttcttgcctgccgatctcctccactggctaaacccagctggatgctaagagtacagtcagcctgcctgctgaggagggaccaccagggaccaccatcagcaagggatccaatgtctttctgcctctgcagaatgaaggttggggcgcggggggcgctctacttcttagggatattgtgggaataaaaggaaataggcaaaaaatgtttttgaaaaacaaagcacatactgcgcacccgtgggccactactgcttttgacccctggctctgtttcatgaagtaatgtcgtgtcattctctttttaggtgctacaggatttctttaggtttgttttctgtccaccatatttcaactcatgtgtgctgtttgttgtgctaaaacaaatatttgctgatgcctgagtgaatagttgaatattttatataagtcaaatttatacgtaatgatttttcttgtaacttagccgtttctcttttacaaactcagaaaacctcagactttgaaaaggccttgaagttcctcacctgaaatctgagaacttggagcgccttaaaaaatctaaaggaaaacaaaacagtgaaagaacatgatatagtcagtgtagagaataaaattatttatgtaattaatattgaggatgcagataacacattgtgaaatcttgcttgtaaaaaatctcgatctgctgaagaaagatgttctctctagagatctttgaaagcataattattgagcttttaaaatgttagaaacaaaagttagacccacacatattctggcgtgtggaagatttgcattccttcccctgcccgccccgcccccacacttgtgagttgtgcctgtgtacgcagttcctgtagcactcggctgggcagaaatcatctttcagcactaagggaacatagttatgatctggaccttctgggagtggtcagtgcccaagaacaggtatgggactccagaaagttctgctctcaaccctattttgaaatagagttacacattgttctacaattatttgagttaataagcagctcttttcaaacgtgattatgcccttccaagtttaaatacactagactttagtgaaagtaattgacctcatctcatttctctcctgttatattaagatcactttcagtaaaaggtagaagcttttgaagtggtgaggaggaggtagaggagggacatagagcagataggggctggaaagtggggtgaggaagagagtggcttctctttggcagagtaccaaggaaaagccctatctgtacagaacctttgtgcctgggaacttgatggctgcaacctgagcctcaacctagtttgcttgcggagccagaagagaagctaaaaaccttcagttaaccaagccagacaccaagaaagttaaaccgaaagagaaccccccaccccccgcaaaaaaaagaagtaaagtgggttaaagtgatatcatgttagcacagaaagagaacataagggtcatctaagttcatctgccccctcttctatttcaaggtgcagaaactaaggcacaagggaccccgtgtcctgctcttgatcacatagctagtgggtgccaagccaggtctagaactctgttctctggggtcacaggctggctcttcatccctctagagagatagctcatctgtgtgcacctgagcccgttgtgtttcggagtcaaagcaaataaaggctcaaactccaagactgttttgcagaccggctgcagtagatatggggggaggagaaacctgctttaaattgcttcaagcaagttgtttctgcaaaggtgttgacttttttctttcaactttctagtgagtcactgcagcctgagctgttatttgtcattatgcaataattcaggaactaactcaagattcttctttttaaattatttgtttatttagagacagagtcttgctctgttgcccaggctggagtgcagtggtgtgatctcggctcactgcagcctctgcctcctgggttcaagcaattctcatgtctcagcctcccgaatagctggtattgcaggctcgtgccaccaccccctgctaatttttgtaattttagtggagacacggtttcgccatgttggccgggctcgtcttgagctcctggcctcaggtgatccgcccgcctcggcctcccaaagtgctgggattgcagccgtgagcctccacacccggcctatttatttatttttaaattggctgctcttagaaaggcataccatgtttctggatgggaaggcttattaattcaccctaatttaatgtataaatttgatgcaatcatagtcacagtcccagtggaattttttaacttggtaagatgttctaaaattaatgagagaacttgaattaccaggtattgaaacactgtaaagccacaatcatgtaaacagtatgttataaccatgggaatagaggtctgtgatacagcagaaaaaagtgaaaaaaagaataactgtattcataaaaatttaaatgtggagtcactgggggaaaggattaaatattcgataatgtagaaacaactcaactatttggagaaatgtaaatttagagccttatctcatgccatataccaaaatactatttagatttgattaaaaaataaaaaaaaaaaaaaaaaaa

SEQ ID NO: 154 - DNA, 2177 bp, Homo sapiens
cgaacgccttcgcgcgatcgccctggaaacgcattctctgcgaccggcagccgccaatgggaagggagtgagtgccacgaacaggccaataaggagggagcagtgcggggtttaaatctgaggctaggctggctcttctcggcgtgctgcggcggaacggctgttggtttctgctgggtgtaggtccttggctggtcgggcctccggtgttctgcttctccccgctgagctgctgcctggtgaagaggaagccatggcgctccgagtcaccaggaactcgaaaattaatgctgaaaataaggcgaagatcaacatggcaggcgcaaagcgcgttcctacggcccctgctgcaacctccaagcccggactgaggccaagaacagctcttggggacattggtaacaaagtcagtgaacaactgcaggccaaaatgcctatgaagaaggaagcaaaaccttcagctactggaaaagtcattgataaaaaactaccaaaacctcttgaaaaggtacctatgctggtgccagtgccagtgtctgagccagtgccagagccagaacctgagccagaacctgagcctgttaaagaagaaaaactttcgcctgagcctattttggttgatactgcctctccaagcccaatggaaacatctggatgtgcccctgcagaagaagacctgtgtcaggctttctctgatgtaattcttgcagtaaatgatgtggatgcagaagatggagctgatccaaacctttgtagtgaatatgtgaaagatatttatgcttatctgagacaacttgaggaagagcaagcagtcagaccaaaatacctactgggtcgggaagtcactggaaacatgagagccatcctaattgactggctagtacaggttcaaatgaaattcaggttgttgcaggagaccatgtacatgactgtctccattattgatcggttcatgcagaataattgtgtgcccaagaagatgctgcagctggttggtgtcactgccatgtttattgcaagcaaatatgaagaaatgtaccctccagaaattggtgactttgcttttgtgactgacaacacttatactaagcaccaaatcagacagatggaaatgaagattctaagagctttaaactttggtctgggtcggcctctacctttgcacttccttcggagagcatctaagattggagaggttgatgtcgagcaacatactttggccaaatacctgatggaactaactatgttggactatgacatggtgcactttcctccttctcaaattgcagcaggagctttttgcttagcactgaaaattctggataatggtgaatggacaccaactctacaacattacctgtcatatactgaagaatctcttcttccagttatgcagcacctggctaagaatgtagtcatggtaaatcaaggacttacaaagcacatgactgtcaagaacaagtatgccacatcgaagcatgctaagatcagcactctaccacagctgaattctgcactagttcaagatttagccaaggctgtggcaaaggtgtaacttgtaaacttgagttggagtactatatttacaaataaaattggcaccatgtgccatctgtacatattactgttgcatttacttttaataaagcttgtggccccttttacttttttatagcttaactaatttgaatgtggttacttcctactgtagggtagcggaaaagttgtcttaaaaggtatggtggggatatttttaaaaactccttttggtttacctggggatccaattgatgtatatgtttatatactgggttcttgttttatatacctggcttttactttattaatatgagttactgaaggtgatggaggtatttgaaaattttacttccataggacatactgcatgtaagccaagtcatggagaatctgctgcatagctctattttaaagtaaaagtctaccaccgaatccctagtccccctgttttctgtttcttcttgtgattgctgccataattctaagttatttacttttaccactatttaagttatcaactttagctagtatcttcaaactttcactttgaaaaatgagaattttatattctaagccagttttcattttggttttgtgttttggttaataaaacaatactcaaatacaaaaaaaaaaaa

SEQ ID NO: 155 - DNA, 2011 bp, Homo sapiens
gcggccgccagcgcggtgtagggggcaggcgcggatcccgccaccgccgcgcgctcggcccgccgactcccggcgccgccgccgccactgccgtcgccgccgccgcctgccgggactggagcgcgccgtccgccgcggacaagaccctggcctcaggccggagcagccccatcatgccgagggagcgcagggagcgggatgcgaaggagcgggacaccatgaaggaggacggcggcgcggagttctcggctcgctccaggaagaggaaggcaaacgtgaccgtttttttgcaggatccagatgaagaaatggccaaaatcgacaggacggcgagggaccagtgtgggagccagccttgggacaataatgcagtctgtgcagacccctgctccctgatccccacacctgacaaagaagatgatgaccgggtttacccaaactcaacgtgcaagcctcggattattgcaccatccagaggctccccgctgcctgtactgagctgggcaaatagagaggaagtctggaaaatcatgttaaacaaggaaaagacatacttaagggatcagcactttcttgagcaacaccctcttctgcagccaaaaatgcgagcaattcttctggattggttaatggaggtgtgtgaagtctataaacttcacagggagaccttttacttggcacaagatttctttgaccggtatatggcgacacaagaaaatgttgtaaaaactcttttacagcttattgggatttcatctttatttattgcagccaaacttgaggaaatctatcctccaaagttgcaccagtttgcgtatgtgacagatggagcttgttcaggagatgaaattctcaccatggaattaatgattatgaaggcccttaagtggcgtttaagtcccctgactattgtgtcctggctgaatgtatacatgcaggttgcatatctaaatgacttacatgaagtgctactgccgcagtatccccagcaaatctttatacagattgcagagctgttggatctctgtgtcctggatgttgactgccttgaatttccttatggtatacttgctgcttcggccttgtatcatttctcgtcatctgaattgatgcaaaaggtttcagggtatcagtggtgcgacatagagaactgtgtcaagtggatggttccatttgccatggttataagggagacggggagctcaaaactgaagcacttcaggggcgtcgctgatgaagatgcacacaacatacagacccacagagacagcttggatttgctggacaaagcccgagcaaagaaagccatgttgtctgaacaaaatagggcttctcctctccccagtgggctcctcaccccgccacagagcggtaagaagcagagcagcgggccggaaatggcgtgaccaccccatccttctccaccaaagacagttgcgcgcctgctccacgttctcttctgtctgttgcagcggaggcgtgcgtttgcttttacagatatctgaatggaagagtgtttcttccacaacagaagtatttctgtggatggcatcaaacagggcaaagtgttttttattgaatgcttataggttttttttaaataagtgggtcaagtacaccagccacctccagacaccagtgcgtgctcccgatgctgctatggaaggtgctacttgacctaagggactcccacaacaacaaaagcttgaagctgtggagggccacggtggcgtggctctcctcgcaggtgttctgggctccgttgtaccaagtggagcaggtggttgcgggcaagcgttgtgcagagcccatagccagctgggcagggggctgccctctccacattatcagttgacagtgtacaatgcctttgatgaactgttttgtaagtgctgctatatctatccattttttaataaagataatactgtttttgaaaaaaaaaaaaaaaaaaaaaaaaaaaaaaaaaaaaaaaaaaaaaaaaaaaaaaaaaaaaaaaaaaaaaaaaaaa

SEQ ID NO: 156 - DNA, 1737 bp, Homo sapiens
gagggcacgggctccgtaggcaccaactgcaaggacccctccccctgcgggcgctcccatggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaaccacaaaaatgcgccagagggttatcacgaacagactgaaagtactctacagccaaaaggccactcctggctccagccggaagacctgccgtttacattccttccctgccaagaccgtatcctggatgcgcctgaaatcgaatgactattaactgaacctgtgggactggcagtccggggaatgtccgggccgggccacggccacgaggtgttccgtgtggagtgcaagctgggacacaccgtgccgcttgtgcacagggccacgcggggaaataatcccggggcgcgcaaagcggcactggcgagagccgcacgggccggtgctgggggtggtacaacaggccaaaacaacacacaaggccaacaagacatacgcgcgctgacaccacggtgcaaagcgctcagacgagtagtaaccggcactgtggttgctgcctccccacctctcccgctctcagcgtaagataaaagaaagaagagcaaaaagcaaagaaagaagacgagacgagacacacaggaacgaacagtaaagcaagctaaagcaaacgcaagaccagacaacagaaatagaaagaaccaacagagaggagacagaacaggacgccagcaacatagcaacaaacgaacagaagagagcactaaacaaaagcagcagcaagacgagacaggagagaaggaggaaggagggccgagcgagcagggagcgcgagcagcgaggcgaagcagcagacaagggcaggcgaagggcaacgagaggaggcaccacacaaaaaggagaggggacaggagaagcagcgagagaagcggaggagcaacaagaggaagaaaaggagagggagaggagggagagagcggaaggaggaagaaacagcacgaggcgacgaaggggggagacgcgggggcaggaaaagacacaggaaggcagcgcggaggaggagaaggggaagcaggaaggagacggaaggagaagagggagaggacagcgcaagagagcgcgcgcggcgacagcgagggacggagcgagagagaggaaacggaaagcgagagggaagaggagaggcaacgcagcgaaccaaccgaaaacagcagaaagagaggagaaggacgcgcaaagaggcaagcgcaagacgacaggaaacgaagcgagagacgagaagccggtgacgagcaggagaaagggaaggcaggagacaggacaggcggaagagagacacgcgagacgcaaagagtgagcagaacgaagcgaagagcaacgcacgagagaaacgac

SEQ ID NO: 157 - DNA, 3053 bp, Homo sapiens
gagcgcggctggagtttgctgctgccgctgtgcagtttgttcaggggcttgtggtggtgagtccgagaggctgcgtgtgagagacgtgagaaggatcctgcactgaggaggtggaaagaagaggattgctcgaggaggcctggggtctgtgaggcagcggagctgggtgaaggctgcgggttccggcgaggcctgagctgtgctgtcgtcatgcctcaaacccgatcccaggcacaggctacaatcagttttccaaaaaggaagctgtctcgggcattgaacaaagctaaaaactccagtgatgccaaactagaaccaacaaatgtccaaaccgtaacctgttctcctcgtgtaaaagccctgcctctcagccccaggaaacgtctgggcgatgacaacctatgcaacactccccatttacctccttgttctccaccaaagcaaggcaagaaagagaatggtccccctcactcacatacacttaagggacgaagattggtatttgacaatcagctgacaattaagtctcctagcaaaagagaactagccaaagttcaccaaaacaaaatactttcttcagttagaaaaagtcaagagatcacaacaaattctgagcagagatgtccactgaagaaagaatctgcatgtgtgagactattcaagcaagaaggcacttgctaccagcaagcaaagctggtcctgaacacagctgtcccagatcggctgcctgccagggaaagggagatggatgtcatcaggaatttcttgagggaacacatctgtgggaaaaaagctggaagcctttacctttctggtgctcctggaactggaaaaactgcctgcttaagccggattctgcaagacctcaagaaggaactgaaaggctttaaaactatcatgctgaattgcatgtccttgaggactgcccaggctgtattcccagctattgctcaggagatttgtcaggaagaggtatccaggccagctgggaaggacatgatgaggaaattggaaaaacatatgactgcagagaagggccccatgattgtgttggtattggacgagatggatcaactggacagcaaaggccaggatgtattgtacacgctatttgaatggccatggctaagcaattctcacttggtgctgattggtattgctaataccctggatctcacagatagaattctacctaggcttcaagctagagaaaaatgtaagccacagctgttgaacttcccaccttataccagaaatcagatagtcactattttgcaagatcgacttaatcaggtatctagagatcaggttctggacaatgctgcagttcaattctgtgcccgcaaagtctctgctgtttcaggagatgttcgcaaagcactggatgtttgcaggagagctattgaaattgtagagtcagatgtcaaaagccagactattctcaaaccactgtctgaatgtaaatcaccttctgagcctctgattcccaagagggttggtcttattcacatatcccaagtcatctcagaagttgatggtaacaggatgaccttgagccaagaaggagcacaagattccttccctcttcagcagaagatcttggtttgctctttgatgctcttgatcaggcagttgaaaatcaaagaggtcactctggggaagttatatgaagcctacagtaaagtctgtcgcaaacagcaggtggcggctgtggaccagtcagagtgtttgtcactttcagggctcttggaagccaggggcattttaggattaaagagaaacaaggaaacccgtttgacaaaggtgtttttcaagattgaagagaaagaaatagaacatgctctgaaagataaagctttaattggaaatatcttagctactggattgccttaaattcttctcttacaccccacccgaaagtattcagctggcatttagagagctacagtcttcattttagtgctttacacattcgggcctgaaaacaaatatgaccttttttacttgaagccaatgaattttaatctatagattctttaatattagcacagaataatatctttgggtcttactatttttacccataaaagtgaccaggtagaccctttttaattacattcactacttctaccacttgtgtatctctagccaatgtgcttgcaagtgtacagatctgtgtagaggaatgtgtgtatatttacctcttcgtttgctcaaacatgagtgggtatttttttgtttgttttttttgttgttgttgtttttgaggcgcgtctcaccctgttgcccaggctggagtgcaatggcgcgttctctgctcactacagcacccgcttcccaggttgaagtgattctcttgcctcagcctcccgagtagctgggattacaggtgcccaccaccgcgcccagctaattttttaatttttagtagagacagggttttaccatgttggccaggctggtcttgaactcctgaccctcaagtgatctgcccaccttggcctccctaagtgctgggattataggcgtgagccaccatgctcagccattaaggtattttgttaagaactttaagtttagggtaagaagaatgaaaatgatccagaaaaatgcaagcaagtccacatggagatttggaggacactggttaaagaatttatttctttgtatagtatactatgttcatggtgcagatactacaacattgtggcattttagactcgttgagtttcttgggcactcccaagggcgttggggtcataaggagactataactctacagattgtgaatatatttattttcaagttgcattctttgtctttttaagcaatcagatttcaagagagctcaagctttcagaagtcaatgtgaaaattccttcctaggctgtcccacagtctttgctgcccttagatgaagccacttgtttcaagatgactactttggggttgggttttcatctaaacacatttttccagtcttattagataaattagtccatatggttggttaatcaagagccttctgggtttggtttggtggcattaaatgg

SEQ ID NO: 158 - DNA, 1843 bp, Homo sapiens
gcggaatggggcgggacttccagtaggaggcggcaagtttgaaaagtgatgacggttgacgtttgctgatttttgactttgcttgtagctgctccccgaactcgccgtcttcctgtcggcggccggcactgtagattaacaggaaacttccaagatggaaactttgtctttccccagatataatgtagctgagattgtgattcatattcgcaataagatcttaacaggagctgatggtaaaaacctcaccaagaatgatctttatccaaatccaaagcctgaagtcttgcacatgatctacatgagagccttacaaatagtatatggaattcgactggaacatttttacatgatgccagtgaactctgaagtcatgtatccacatttaatggaaggcttcttaccattcagcaatttagttactcatctggactcatttttgcctatctgccgggtgaatgactttgagactgctgatattctatgtccaaaagcaaaacggacaagtcggtttttaagtggcattatcaactttattcacttcagagaagcatgccgtgaaacgtatatggaatttctttggcaatataaatcctctgcggacaaaatgcaacagttaaacgccgcacaccaggaggcattaatgaaactggagagacttgattctgttccagttgaagagcaagaagagttcaagcagctttcagatggaattcaggagctacaacaatcactaaatcaggattttcatcaaaaaacgatagtgctgcaagagggaaattcccaaaagaagtcaaatatttcagagaaaaccaagcgtttgaatgaactaaaattgtcggtggtttctttgaaagaaatacaagagagtttgaaaacaaaaattgtggattctccagagaagttaaagaattataaagaaaaaatgaaagatacggtccagaagcttaaaaatgccagacaagaagtggtggagaaatatgaaatctatggagactcagttgactgcctgccttcatgtcagttggaagtgcagttatatcaaaagaaaatacaggacctttcagataatagggaaaaattagccagtatcttaaaggagagcctgaacttggaggaccaaattgagagtgatgagtcagaactgaagaaattgaagactgaagaaaattcgttcaaaagactgatgattgtgaagaaggaaaaacttgccacagcacaattcaaaataaataagaagcatgaagatgttaagcaatacaaacgcacagtaattgaggattgcaataaagttcaagaaaaaagaggtgctgtctatgaacgagtaaccacaattaatcaagaaatccaaaaaattaaacttggaattcaacaactaaaagatgctgctgaaagggagaaactgaagtcccaggaaatatttctaaacttgaaaactgctttggagaaataccacgacggtattgaaaaggcagcagaggactcctatgctaagatagatgagaagacagctgaactgaagaggaagatgttcaaaatgtcaacctgattaacaaaattacatgtctttttgtaaatggcttgccatcttttaattttctatttagaaagaaaagttgaagcgaatggaagtatcagaagtaccaaataatgttggcttcatcagtttttatacactctcataagtagttaataagatgaatttaatgtaggcttttattaatttataattaaaataacttgtgcagctattcatgtctctactctgccccttgttgtaaatagtttgagtaaaacaaaactagttacctttgaaatatatatatttttttctgttactatc

SEQ ID NO: 159 - DNA, 3686 bp, Homo sapiens
ggctagcgcgggaggtggagaaagaggcttgggcggccccgctgtagccgcgtgtgggaggacgcacgggcctgcttcaaagctttgggataacagcgcctccgggggataatgaatgcggagcctccgttttcagtcgacttcagatgtgtctccacttttttccgctgtagccgcaaggcaaggaaacatttctcttcccgtactgaggaggctgaggagtgcactgggtgttcttttctcctctaacccagaactgcgagacagaggctgagtccctgtaaagaacagctccagaaaagccaggagagcgcaggagggcatccgggaggccaggaggggttcgctggggcctcaaccgcacccacatcggtcccacctgcgagggggcgggacctcgtggcgctggaccaatcagcacccacctgcgctcacctggcctcctcccgctggctcccgggggctgcggtgctcaaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatagtgaccgaccagaatgaccacaagcccaagtttacccaggacaccttccgagggagtgtcttagagggagtcctaccaggtacttctgtgatgcagatgacagccacagatgaggatgatgccatctacacctacaatggggtggttgcttactccatccatagccaagaaccaaaggacccacacgacctcatgttcacaattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaacgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaaaggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctaggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccaaaaaaaaaaaaaaaa

SEQ ID NO: 160 - DNA, 10316 bp, Homo sapiens
gagaccagaagcgggcgaattgggcaccggtggcggctgcgggcagtttgaattagactctgggctccagcccgccgaagccgcgccagaactgtactctccgagaggtcgttttcccgtccccgagagcaagtttatttacaaatgttggagtaataaagaaggcagaacaaaatgagctgggctttggaagaatggaaagaagggctgcctacaagagctcttcagaaaattcaagagcttgaaggacagcttgacaaactgaagaaggaaaagcagcaaaggcagtttcagcttgacagtctcgaggctgcgctgcagaagcaaaaacagaaggttgaaaatgaaaaaaccgagggtacaaacctgaaaagggagaatcaaagattgatggaaatatgtgaaagtctggagaaaactaagcagaagatttctcatgaacttcaagtcaaggagtcacaagtgaatttccaggaaggacaactgaattcaggcaaaaaacaaatagaaaaactggaacaggaacttaaaaggtgtaaatctgagcttgaaagaagccaacaagctgcgcagtctgcagatgtctctctgaatccatgcaatacaccacaaaaaatttttacaactccactaacaccaagtcaatattatagtggttccaagtatgaagatctaaaagaaaaatataataaagaggttgaagaacgaaaaagattagaggcagaggttaaagccttgcaggctaaaaaagcaagccagactcttccacaagccaccatgaatcaccgcgacattgcccggcatcaggcttcatcatctgtgttctcatggcagcaagagaagaccccaagtcatctttcatctaattctcaaagaactccaattaggagagatttctctgcatcttacttttctggggaacaagaggtgactccaagtcgatcaactttgcaaatagggaaaagagatgctaatagcagtttctttgacaattctagcagtcctcatcttttggatcaattaaaagcgcagaatcaagagctaagaaacaagattaatgagttggaactacgcctgcaaggacatgaaaaagaaatgaaaggccaagtgaataagtttcaagaactccaactccaactggagaaagcaaaagtggaattaattgaaaaagagaaagttttgaacaaatgtagggatgaactagtgagaacaacagcacaatacgaccaggcgtcaaccaagtatactgcattggaacaaaaactgaaaaaattgacggaagatttgagttgtcagcgacaaaatgcagaaagtgccagatgttctctggaacagaaaattaaggaaaaagaaaaggagtttcaagaggagctctcccgtcaacagcgttctttccaaacactggaccaggagtgcatccagatgaaggccagactcacccaggagttacagcaagccaagaatatgcacaacgtcctgcaggctgaactggataaactcacatcagtaaagcaacagctagaaaacaatttggaagagtttaagcaaaagttgtgcagagctgaacaggcgttccaggcgagtcagatcaaggagaatgagctgaggagaagcatggaggaaatgaagaaggaaaacaacctccttaagagtcactctgagcaaaaggccagagaagtctgccacctggaggcagaactcaagaacatcaaacagtgtttaaatcagagccagaattttgcagaagaaatgaaagcgaagaatacctctcaggaaaccatgttaagagatcttcaagaaaaaataaatcagcaagaaaactccttgactttagaaaaactgaagcttgctgtggctgatctggaaaagcagcgagattgttctcaagaccttttgaagaaaagagaacatcacattgaacaacttaatgataagttaagcaagacagagaaagagtccaaagccttgctgagtgctttagagttaaaaaagaaagaatatgaagaattgaaagaagagaaaactctgttttcttgttggaaaagtgaaaacgaaaaacttttaactcagatggaatcagaaaaggaaaacttgcagagtaaaattaatcacttggaaacttgtctgaagacacagcaaataaaaagtcatgaatacaacgagagagtaagaacgctggagatggacagagaaaacctaagtgtcgagatcagaaaccttcacaacgtgttagacagtaagtcagtggaggtagagacccagaaactagcttatatggagctacagcagaaagctgagttctcagatcagaaacatcagaaggaaatagaaaatatgtgtttgaagacttctcagcttactgggcaagttgaagatctagaacacaagcttcagttactgtcaaatgaaataatggacaaagaccggtgttaccaagacttgcatgccgaatatgagagcctcagggatctgctaaaatccaaagatgcttctctggtgacaaatgaagatcatcagagaagtcttttggcttttgatcagcagcctgccatgcatcattcctttgcaaatataattggagaacaaggaagcatgccttcagagaggagtgaatgtcgtttagaagcagaccaaagtccgaaaaattctgccatcctacaaaatagagttgattcacttgaattttcattagagtctcaaaaacagatgaactcagacctgcaaaagcagtgtgaagagttggtgcaaatcaaaggagaaatagaagaaaatctcatgaaagcagaacagatgcatcaaagttttgtggctgaaacaagtcagcgcattagtaagttacaggaagacacttctgctcaccagaatgttgttgctgaaaccttaagtgcccttgagaacaaggaaaaagagctgcaacttttaaatgataaggtagaaactgagcaggcagagattcaagaattaaaaaagagcaaccatctacttgaagactctctaaaggagctacaacttttatccgaaaccctaagcttggagaagaaagaaatgagttccatcatttctctaaataaaagggaaattgaagagctgacccaagagaatgggactcttaaggaaattaatgcatccttaaatcaagagaagatgaacttaatccagaaaagtgagagttttgcaaactatatagatgaaagggagaaaagcatttcagagttatctgatcagtacaagcaagaaaaacttattttactacaaagatgtgaagaaaccggaaatgcatatgaggatcttagtcaaaaatacaaagcagcacaggaaaagaattctaaattagaatgcttgctaaatgaatgcactagtctttgtgaaaataggaaaaatgagttggaacagctaaaggaagcatttgcaaaggaacaccaagaattcttaacaaaattagcatttgctgaagaaagaaatcagaatctgatgctagagttggagacagtgcagcaagctctgagatctgagatgacagataaccaaaacaattctaagagcgaggctggtggtttaaagcaagaaatcatgactttaaaggaagaacaaaacaaaatgcaaaaggaagttaatgacttattacaagagaatgaacagctgatgaaggtaatgaagactaaacatgaatgtcaaaatctagaatcagaaccaattaggaactctgtgaaagaaagagagagtgagagaaatcaatgtaattttaaacctcagatggatcttgaagttaaagaaatttctctagatagttataatgcgcagttggtgcaattagaagctatgctaagaaataaggaattaaaacttcaggaaagtgagaaggagaaggagtgcctgcagcatgaattacagacaattagaggagatcttgaaaccagcaatttgcaagacatgcagtcacaagaaattagtggccttaaagactgtgaaatagatgcggaagaaaagtatatttcagggcctcatgagttgtcaacaagtcaaaacgacaatgcacaccttcagtgctctctgcaaacaacaatgaacaagctgaatgagctagagaaaatatgtgaaatactgcaggctgaaaagtatgaactcgtaactgagctgaatgattcaaggtcagaatgtatcacagcaactaggaaaatggcagaagaggtagggaaactactaaatgaagttaaaatattaaatgatgacagtggtcttctccatggtgagttagtggaagacataccaggaggtgaatttggtgaacaaccaaatgaacagcaccctgtgtctttggctccattggacgagagtaattcctacgagcacttgacattgtcagacaaagaagttcaaatgcactttgccgaattgcaagagaaattcttatctttacaaagtgaacacaaaattttacatgatcagcactgtcagatgagctctaaaatgtcagagctgcagacctatgttgactcattaaaggccgaaaatttggtcttgtcaacgaatctgagaaactttcaaggtgacttggtgaaggagatgcagctgggcttggaggaggggctcgttccatccctgtcatcctcttgtgtgcctgacagctctagtcttagcagtttgggagactcctccttttacagagctcttttagaacagacaggagatatgtctcttttgagtaatttagaaggggctgtttcagcaaaccagtgcagtgtagatgaagtattttgcagcagtctgcaggaggagaatctgaccaggaaagaaaccccttcggccccagcgaagggtgttgaagagcttgagtccctctgtgaggtgtaccggcagtccctcgagaagctagaagagaaaatggaaagtcaagggattatgaaaaataaggaaattcaagagctcgagcagttattaagttctgaaaggcaagagcttgactgccttaggaagcagtatttgtcagaaaatgaacagtggcaacagaagctgacaagcgtgactctggagatggagtccaagttggcggcagaaaagaaacagacggaacaactgtcacttgagctggaagtagcacgactccagctacaaggtctggacttaagttctcggtctttgcttggcatcgacacagaagatgctattcaaggccgaaatgagagctgtgacatatcaaaagaacatacttcagaaactacagaaagaacaccaaagcatgatgttcatcagatttgtgataaagatgctcagcaggacctcaatctagacattgagaaaataactgagactggtgcagtgaaacccacaggagagtgctctggggaacagtccccagataccaattatgagcctccaggggaagataaaacccagggctcttcagaatgcatttctgaattgtcattttctggtcctaatgctttggtacctatggatttcctggggaatcaggaagatatccataatcttcaactgcgggtaaaagagacatcaaatgagaatttgagattacttcatgtgatagaggaccgtgacagaaaagttgaaagtttgctaaatgaaatgaaagaattagactcaaaactccatttacaggaggtacaactaatgaccaaaattgaagcatgcatagaattggaaaaaatagttggggaacttaagaaagaaaactcagatttaagtgaaaaattggaatatttttcttgtgatcaccaggagttactccagagagtagaaacttctgaaggcctcaattctgatttagaaatgcatgcagataaatcatcacgtgaagatattggagataatgtggccaaggtgaatgacagctggaaggagagatttcttgatgtggaaaatgagctgagtaggatcagatcggagaaagctagcattgagcatgaagccctctacctggaggctgacttagaggtagttcaaacagagaagctatgtttagaaaaagacaatgaaaataagcagaaggttattgtctgccttgaagaagaactctcagtggtcacaagtgagagaaaccagcttcgtggagaattagatactatgtcaaaaaaaaccacggcactggatcagttgtctgaaaaaatgaaggagaaaacacaagagcttgagtctcatcaaagtgagtgtctccattgcattcaggtggcagaggcagaggtgaaggaaaagacggaactccttcagactttgtcctctgatgtgagtgagctgttaaaagacaaaactcatctccaggaaaagctgcagagtttggaaaaggactcacaggcactgtctttgacaaaatgtgagctggaaaaccaaattgcacaactgaataaagagaaagaattgcttgtcaaggaatctgaaagcctgcaggccagactgagtgaatcagattatgaaaagctgaatgtctccaaggccttggaggccgcactggtggagaaaggtgagttcgcattgaggctgagctcaacacaggaggaagtgcatcagctgagaagaggcatcgagaaactgagagttcgcattgaggccgatgaaaagaagcagctgcacatcgcagagaaactgaaagaacgcgagcgggagaatgattcacttaaggataaagttgagaaccttgaaagggaattgcagatgtcagaagaaaaccaggagctagtgattcttgatgccgagaattccaaagcagaagtagagactctaaaaacacaaatagaagagatggccagaagcctgaaagtttttgaattagaccttgtcacgttaaggtctgaaaaagaaaatctgacaaaacaaatacaagaaaaacaaggtcagttgtcagaactagacaagttactctcttcatttaaaagtctgttagaagaaaaggagcaagcagagatacagatcaaagaagaatctaaaactgcagtggagatgcttcagaatcagttaaaggagctaaatgaggcagtagcagccttgtgtggtgaccaagaaattatgaaggccacagaacagagtctagacccaccaatagaggaagagcatcagctgagaaatagcattgaaaagctgagagcccgcctagaagctgatgaaaagaagcagctctgtgtcttacaacaactgaaggaaagtgagcatcatgcagatttacttaagggtagagtggagaaccttgaaagagagctagagatagccaggacaaaccaagagcatgcagctcttgaggcagagaattccaaaggagaggtagagaccctaaaagcaaaaatagaagggatgacccaaagtctgagaggtctggaattagatgttgttactataaggtcagaaaaagaaaatctgacaaatgaattacaaaaagagcaagagcgaatatctgaattagaaataataaattcatcatttgaaaatattttgcaagaaaaagagcaagagaaagtacagatgaaagaaaaatcaagcactgccatggagatgcttcaaacacaattaaaagagctcaatgagagagtggcagccctgcataatgaccaagaagcctgtaaggccaaagagcagaatcttagtagtcaagtagagtgtcttgaacttgagaaggctcagttgctacaaggccttgatgaggccaaaaataattatattgttttgcaatcttcagtgaatggcctcattcaagaagtagaagatggcaagcagaaactggagaagaaggatgaagaaatcagtagactgaaaaatcaaattcaagaccaagagcagcttgtctctaaactgtcccaggtggaaggagagcaccaactttggaaggagcaaaacttagaactgagaaatctgacagtggaattggagcagaagatccaagtgctacaatccaaaaatgcctctttgcaggacacattagaagtgctgcagagttcttacaagaatctagagaatgagcttgaattgacaaaaatggacaaaatgtcctttgttgaaaaagtaaacaaaatgactgcaaaggaaactgagctgcagagggaaatgcatgagatggcacagaaaacagcagagctgcaagaagaactcagtggagagaaaaataggctagctggagagttgcagttactgttggaagaaataaagagcagcaaagatcaattgaaggagctcacactagaaaatagtgaattgaagaagagcctagattgcatgcacaaagaccaggtggaaaaggaagggaaagtgagagaggaaatagctgaatatcagctacggcttcatgaagctgaaaagaaacaccaggctttgcttttggacacaaacaaacagtatgaagtagaaatccagacataccgagagaaattgacttctaaagaagaatgtctcagttcacagaagctggagatagaccttttaaagtctagtaaagaagagctcaataattcattgaaagctactactcagattttggaagaattgaagaaaaccaagatggacaatctaaaatatgtaaatcagttgaagaaggaaaatgaacgtgcccaggggaaaatgaagttgttgatcaaatcctgtaaacagctggaagaggaaaaggagatactgcagaaagaactctctcaacttcaagctgcacaggagaagcagaaaacaggtactgttatggataccaaggtcgatgaattaacaactgagatcaaagaactgaaagaaactcttgaagaaaaaaccaaggaggcagatgaatacttggataagtactgttccttgcttataagccatgaaaagttagagaaagctaaagagatgttagagacacaagtggcccatctgtgttcacagcaatctaaacaagattcccgagggtctcctttgctaggtccagttgttccaggaccatctccaatcccttctgttactgaaaagaggttatcatctggccaaaataaagcttcaggcaagaggcaaagatccagtggaatatgggagaatggtagaggaccaacacctgctaccccagagagcttttctaaaaaaagcaagaaagcagtcatgagtggtattcaccctgcagaagacacggaaggtactgagtttgagccagagggacttccagaagttgtaaagaaagggtttgctgacatcccgacaggaaagactagcccatatatcctgcgaagaacaaccatggcaactcggaccagcccccgcctggctgcacagaagttagcgctatccccactgagtctcggcaaagaaaatcttgcagagtcctccaaaccaacagctggtggcagcagatcacaaaaggtcaaagttgctcagcggagcccagtagattcaggcaccatcctccgagaacccaccacgaaatccgtcccagtcaataatcttcctgagagaagtccgactgacagccccagagagggcctgagggtcaagcgaggccgacttgtccccagccccaaagctggactggagtccaacggcagtgagaactgtaaggtccagtgaaggcactttgtgtgtcagtacccctgggaggtgccagtcattgaatagataaggctgtgcctacaggacttctctttagtcagggcatgctttattagtgaggagaaaacaattccttagaagtcttaaatatattgtactctttagatctcccatgtgtaggtattgaaaaagtttggaagcactgatcacctgttagcattgccattcctctactgcaatgtaaatagtataaagctatgtatataaagctttttggtaatatgttacaattaaaatgacaagcactatatcacaatctctgtttgtatgtgggttttacactaaaaaaatgcaaaacacattttattcttctaattaacagctcctaggaaaatgtagacttttgctttatgatattctatctgtagtatgaggcatggaatagttttgtatcgggaatttctcagagctgagtaaaatgaaggaaaagcatgttatgtgtttttaaggaaaatgtgcacacatatacatgtaggagtgtttatctttctcttacaatctgttttagacatctttgcttatgaaacctgtacatatgtgtgtgtgggtatgtgtttatttccagtgagggctgcaggcttcctagaggtgtgctataccatgcgtctgtcgttgtgcttttttctgtttttagaccaattttttacagttctttggtaagcattgtcgtatctggtgatggattaacatatagcctttgttttctaataaaatagtcgccttcgttttctgtaaaaaaaaaaaaaaaaaaaaaa

SEQ ID NO: 161 - DNA, 2639 bp, Homo sapiens
ggcacgaggggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcatgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggtttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgttttaaaaaaaaaaaaaaa

SEQ ID NO: 162 - DNA, 2632 bp, Homo sapiens
ggcggctgagcctgagcggggatgtagaggcggcggcagcagaggcggcactggcggcaagagcagacgcccgagccgagcgagaagagcggcagagccttatcccctgaagccgggccccgcgtcccagccctgcccagcccgcgcccagccatgcgcgccgcctgctgagtccgggcgccgcacgctgagccctccgcccgcgagccgcgctcagctcgggggtgattagttgctttttgttgttttttaatttgggccgcggggagggggaggaggggcaggtgctgcaggctcccccccctccccgcctcgggccagccgcggcggcgcgactcgggctccggacccgggcactgctggcggctggagcggagcgcaccgcggcggtggtgcccagagcggagcgcagctccctgccccgcccctccccctcggcctcgcggcgacggcggcggtggcggcttggacgactcggagagccgagtgaagacatttccacctggacacctgaccatgtgcctgccctgagcagcgaggcccaccaggcatctctgttgtgggcagcagggccaggtcctggtctgtggaccctcggcagttggcaggctccctctgcagtggggtctgggcctcggccccaccatgtcgagcctcggcggtggctcccaggatgccggcggcagtagcagcagcagcaccaatggcagcggtggcagtggcagcagtggcccaaaggcaggagcagcagacaagagtgcagtggtggctgccgccgcaccagcctcagtggcagatgacacaccaccccccgagcgtcggaacaagagcggtatcatcagtgagcccctcaacaagagcctgcgccgctcccgcccgctctcccactactcttcttttggcagcagtggtggtagtggcggtggcagcatgatgggcggagagtctgctgacaaggccactgcggctgcagccgctgcctccctgttggccaatgggcatgacctggcggcggccatggcggtggacaaaagcaaccctacctcaaagcacaaaagtggtgctgtggccagcctgctgagcaaggcagagcgggccacggagctggcagccgagggacagctgacgctgcagcagtttgcgcagtccacagagatgctgaagcgcgtggtgcaggagcatctcccgctgatgagcgaggcgggtgctggcctgcctgacatggaggctgtggcaggtgccgaagccctcaatggccagtccgacttcccctacctgggcgctttccccatcaacccaggcctcttcattatgaccccggcaggtgtgttcctggccgagagcgcgctgcacatggcgggcctggctgagtaccccatgcagggagagctggcctctgccatcagctccggcaagaagaagcggaaacgctgcggcatgtgcgcgccctgccggcggcgcatcaactgcgagcagtgcagcagttgtaggaatcgaaagactggccatcagatttgcaaattcagaaaatgtgaggaactcaaaaagaagccttccgctgctctggagaaggtgatgcttccgacgggagccgccttccggtggtttcagtgacggcggcggaacccaaagctgccctctccgtgcaatgtcactgctcgtgtggtctccagcaagggattcgggcgaagacaaacggatgcacccgtctttagaaccaaaaatattctctcacagatttcattcctgtttttatatatatattttttgttgtcgttttaacatctccacgtccctagcataaaaagaaaaagaaaaaaatttaaactgctttttcggaagaacaacaacaaaaaagaggtaaagacgaatctataaagtaccgagacttcctgggcaaagaatggacaatcagtttccttcctgtgtcgatgtcgatgttgtctgtgcaggagatgcagtttttgtgtagagaatgtaaattttctgtaaccttttgaaatctagttactaataagcactactgtaatttagcacagtttaactccaccctcatttaaacttcctttgattctttccgaccatgaaatagtgcatagtttgcctggagaatccactcacgttcataaagagaatgttgatggcgccgtgtagaagccgctctgtatccatccacgcgtgcagagctgccagcagggagctcacagaaggggagggagcaccaggccagctgagctgcacccacagtcccgagactgggatcccccaccccaacagtgattttggaaaaaaaaatgaaagttctgttcgtttatccattgcgatctggggagccccatctcgatatttccaatcctggctacttttcttagagaaaataagtcctttttttctggccttgctaatggcaacagaagaaagggcttctttgcgtggtcccctgctggtgggggtgggtccccagggggccccctgcggcctgggcccccctgcccacggccagcttcctgctgatgaacatgctgtttgtattgttttaggaaaccaggctgttttgtgaataaaacgaatgcatgtttgtgtcacgaaaaaaaaaaaaaaaaaaaaaaaaaaaaaaaaaaaaaaaaaaaaa

SEQ ID NO: 163 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 164 - DNA, 4816 bp, Homo sapiens
gttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 165 - DNA, 6466 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 166 - DNA, 3478 bp, Homo sapiens
aaattgaaaggtcagcctttcgcgcgctgtgtaggcaagttacccgtgttctgcgttgccggccgtgggtgctctggccacagtgagttaggggcgtcggagcgggtttctccaaccgcaatcggctccgctcaaggggaggaggagagtcccttctcggaaggcctaaggaaacgtgtcgtctggaatgggcttgggggccacgcctgcacatctccgcgagacagagggataaagtgaagatggtgctgttattgttacctcgagtgccacatgcgacctctgagatatgtacacagtcattcttactatcgcactcagccattcttactacgctaaagaagaaataattattcgaggatatttgcctggcccagaagaaacttatgtaaatttcatgaactattatatccgttttcctcggagtgagagaaaactctttttagatatcatctgagagaactagtgaatcccagtcactgagtggagttgagagtctaagaacctctgaaatttgagaactgctggaccagagcctttagagctctgataaggtgtcaacagggtagttaatttggcaccatggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaaatgcagactgctgcaaagcttttgcctgcaagagaatctgatcaatttgaagtccctgtttgggaatgaggcacttatcagcatgaagaattttttctcattctgtgccattttaaaaatagaatacattttgtatattaactttataattgggttgtggtttttttgctcagctttttatatttttataagaagctaaatagaagaataattgtatctctgacaggtttttggaggttttagtgttaattgggaaaatcctctggagtttataaaagtctactctaaatatttctgtaatgttgtcaagtagaaagatagtaaatggagaaactacaaaaaaaaaaaaaaaaaa

SEQ ID NO: 167 - DNA, 5192 bp, Homo sapiens
ccatgacctgccttgagaaggggcaggggaagccagatggactggaagtggagtggcagtgaccaaggaggaggaggtgtgataggcttcccacgcagggtagatccagagacaccagtgccacccataggcccctaggactgcagtggtcacccgattcctttgtcccagctgagactcagttctgagtgttctattttggggaacagaggcgtccttggtagcatttggaagaggatagccagctggggtgtgtgtacatcacagcctgacagtaacagcatccgaaccagaggtgactggctaagggcagacccagggcaacaggttaaccgttctagggccgggcacagggaggagaacattccaacactctgtgtgcccagtgccgacgcacgttctctcttttatcctcaaaacagtcctatgaggatataagccagagagagacagagacaaggaattacaagttggtgagagtcaggatttgaacttggctctggcagatggaaaattagggtctgtattctttacaaaaccgtgtgtgcctcagatggagttggtgcataacaagcagaggtatccagggtcgcggtcctgcttgccacggaaggggccgccttgtcagttgtgaccacccagccctggaaatgtcagtaatgctgtaaggagtggggatcggatcagatgccatccagatgctgaagtttgaccttgtgtcatttttcactttcttttttggctcttctgcaatcaattcatttatttagcaaaaaagaaattatgtgtgccgagagcatgcagaagatatgtctccgttctctgcttccctccaaaaaagaatcccaaaactgctttctgtgaacgtgtgccagggtcccagcaggactcagggagagcaggaagcccagcccagaccccttgcacaacctaccgtggggaggccttaggctctggctactacagagctggttccagtctgcactgccacagcctggccagggacttggacacatctgctggccacttcctgtctcagtttccttatctgcaaaataagggaaaagcccccacaaaggtgcacgtgtagcaggagctcttttccctccctattttaggaaggcagttggtgggaagtccagcttgggtccctgagagctgtgagaaggagatgcggctgctgctggccctgttgggggtcctgctgagtgtgcctgggcctccagtcttgtccctggaggcctctgaggaagtggagcttggtatggcttctgaggtgggagagggtggcaggggtgggaagagtgggcaccaggagggggctgctgggctgagcaaagctggaaaggatccttgcccaggccctgagaaggtggcggcagggcagggctcaaccactgagactcagtcagtgcctggcttccagcaagcattcatctatcactgtgtctgcgagagaggactggccttgcagggcgcagggccctaagctgggctgcagagctggtggtgagctccttgcctgggtgtgtgtgcgtgtgtgtgtgtgttctgtgcactgggtgtgtgacctaggaggtccaggcagcatgtgtggtataagcattatgagggtgatatgccccggtgcagcatgaccctgtatgtggcaccaacagcatgtgccttgtgtgtgtgtgtgtccgtatgtgtgtgtgtgtatgcgtgtgtgtgtgtgtgtgtgtgtgtcttggccactgtcatgtgcactaaatgctgtgtgtgtgacatgccccaagagtgtggcatttgccctgggtgtggcatccgcagcatgtggctgtgtgggtgtcaaggagtggtggctccttcagcatgcgttgcgaagtgcttgtgccctgcatgtgcggtgtgttctctgtacacaggaggctgcctcagatggggctgcggggtctgctgacctctgccctctgcccacagagccctgcctggctcccagcctggagcagcaagagcaggagctgacagtagcccttgggcagcctgtgcggctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacctctcgaataggcacagttacccccagcaaggtcagtaggtctccaaggacttgtgtccccgctgctgctcatctgatcactgagaagaggaggcctgtgtgggaacacacggtcattctaggggccttcccctgccctccagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgttataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccaggtgagcacctgaagggccaggagatgctgcgagatgcccctctgggccagcagtgggggctgtggcctgttgggtggtcagtctctgttggcctgtggggtctggcctggggggcagtgtgtggatttgtgggtttgagctgtatgacagcccctctgtgcctctccacacgtggccgtccatgtgaccgtctgctgaggtgtgggtgcctgggactgggcataactacagcttcctccgtgtgtgtccccacatatgttgggagctgggagggactgagttagggtgcacggggcggccagtctcaccactgaccagtttgtctgtctgtgtgtgtccatgtgcgagggcagaggaggaccccacatggaccgcagcagcgcccgaggccaggtatacggacatcatcctgtacgcgtcgggctccctggccttggctgtgctcctgctgctggccaggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccttggcgcttagtccccatcccgggtttggctgagcctggctggagagctgctatgctaaacctcctgcctcccaataccagcaggaggttctgggcctctgaaccccctttccccacacctccccctgctgctgctgccccagcgtcttgacgggagcattggcccctgagcccagagaagctggaagcctgccgaaaacaggagcaaatggcgttttataaattatttttttgaaat

SEQ ID NO: 168 - DNA, 3124 bp, Homo sapiens
taagatccacatcagctcaactgcacttgcctcgcagaggcagcccgctcacttcccgcggaggcgctccccggcgccgcgctccgcggcagccgcctgcccccggcgctgcccccgcccgccgcgccgccgccgccgccgcgcacgccgcgccccgcagctctgggcttcctcttcgcccgggtggcgttgggcccgcgcgggcgctcgggtgactgcagctgctcagctcccctcccccgccccgcgccgcgcggccgcccgtcgcttcgcacagggctggatggttgtattgggcagggtggctccaggatgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctagctcccgggactggggggtttgtctggcatagccatgctggtagcaagagagaaaaaatcaacagcaaacaaaaccacacaaaccaaaccgtcaacagcataataaaatcccaacaactatttttatttcatttttcatgcacaacctttcccccagtgcaaaagactgttactttattattgtattcaaaattcattgtgtatattactacaaagacaaccccaaaccaatttttttcctgcgaagtttaatgatccacaagtgtatatatgaaattctcctccttccttgcccccctctctttcttccctctttcccctccagacattctagtttgtggagggttatttaaaaaaacaaaaaaggaagatggtcaagtttgtaaaatatttgtttgtgctttttccccctccttacctgaccccctacgagtttacaggtctgtggcaatactcttaaccataagaattgaaatggtgaagaaacaagtatacactagaggctcttaaaagtattgaaagacaatactgctgttatatagcaagacataaacagattataaacatcagagccatttgcttctcagtttacatttctgatacatgcagatagcagatgtctttaaatgaaatacatgtatattgtgtatggacttaattatgcacatgctcagatgtgtagacatcctccgtatatttacataacatatagaggtaatagataggtgatatacatgatacattctcaagagttgcttgaccgaaagttacaaggaccccaacccctttgtcctctctacccacagatggccctgggaatcaattcctcaggaattgccctcaagaactctgcttcttgctttgcagagtgccatggtcatgtcattctgaggtcacataacacataaaattagtttctatgagtgtataccatttaaagaatttttttttcagtaaaagggaatattacaatgttggaggagagataagttatagggagctggatttcaaaacgtggtccaagattcaaaaatcctattgatagtggccattttaatcattgccatcgtgtgcttgtttcatccagtgttatgcactttccacagttggacatggtgttagtatagccagacgggtttcattattatttctctttgctttctcaatgttaatttattgcatggtttattctttttctttacagctgaaattgctttaaatgatggttaaaattacaaattaaattgttaatttttatcaatgtgattgtaattaaaaatattttgatttaaataacaaaaataataccagattttaagccgtggaaaatgttcttgatcatttgcagttaaggactttaaataaatcaaatgttaacaaaaaaaaaaaaaaaa

SEQ ID NO: 169 - DNA, 3452 bp, Homo sapiens
atgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcggtgaaggacaaggaggagaaggacaggctgcacctcaaggagccgcccccgcccggccgccagcccccgcccgcgccgccggagcaggccgacggcaacgcgcccggtccgcagccgccgcccgtgcgcatccaggacatcaagaccgagaacggtacgtgcccctcgccgccccagcccctgtccccggccgccgccctgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacgaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcgagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttttgacacaccctcaaagccgaactaaatcgaaccccaaagcaggaaaagctaaaggaacccatcaaggcaaaatcgaaactaaaaaaaaaaaatccaattaaaaaaaacccctgagaatattcaccacaccagcgaacagaatatccctccaaaaattcagctcaccagcaccagcacgaagaaaactctattttcttaaccgattaattcagagccacctccactttgccttgtctaaataaacaaacccgtaaactgttttatacagagacagcaaaatcttggtttattaaaggacagtgttactccagataacacgtaagtttcttcttgcttttcagagacctgctttcccctcctcccgtctcccctctcttgccttcttccttgcctctcacctgtaagatattattttatcctatgttgaagggagggggaaagtccccgtttatgaaagtcgctttctttttattcatggacttgttttaaaatgtaaattgcaacatagtaatttatttttaatttgtagttggatgtcgtggaccaaacgccagaaagtgttcccaaaacctgacgttaaattgcctgaaactttaaattgtgctttttttctcattataaaaagggaaactgtattaatcttattctatcctcttttctttctttttgttgaacatattcattgtttgtttattaataaattaccattcagtttgaatgagacctatatgtctggatactttaatagagctttaattattacgaaaaaagatttcagagataaaacactagaagttacctattctccacctaaatctctgaaaaatggagaaaccctctgactagtccatgtcaaattttactaaaagtctttttgtttagatttattttcctgcagcatcttctgcaaaatgtactatatagtcagcttgctttgaggctagtaaaaagatatttttctaaacagattggagttggcatataaacaaatacgttttctcactaatgacagtccatgattcggaaattttaagcccatgaatcagccgcggtcttaccacggtgatgcctgtgtgccgagagatgggactgtgcggccagatatgcacagataaatatttggcttgtgtattccatataaaattgcagtgcatattatacatccctgtgagccagatgctgaatagatattttcctattatttcagtcctttataaaaggaaaaataaaccagtttttaaatgtatgtatataattctcccccatttacaatccttcatgtattacatagaaggattgcttttttaaaaatatactgcgggttggaaagggatatttaatctttgagaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagaggcaacacttaagcagtaattgctgttgcttgttgtcaaaatttgatcattgttaaaggattgctgcaaataaatacactttaatttcagtcaaaaa

SEQ ID NO: 170 - DNA, 1749 bp, Homo sapiens
gtggcctcgaggtggtggcagggccgccccctgcagtccggagacgaacgcacggaccgggcctccggaggcaggttcggctggaaggaaccgctctcgcttcgtcctacacttgcgcaaatgtctccgagcttactcacatagcatattggtatatcaaaatgaaatgcaaggaaccaaaaataacataattgaaggcagtaaaagtgaaattaaataggaagatcatcagtcaaggaagacccactggagaggacagaaaatgaagcagtgttttatcatgtgtatttcagcaggtcttcttgaaatttaactaaaaatatgactgctctctcttcagagaactgctcttttcagtaccagttacgtcaaacaaaccagcccctagacgttaactatctgctattcttgatcatacttgggaaaatattattaaatatccttacactaggaatgagaagaaaaaacacctgtcaaaattttatggaatatttttgcatttcactagcattcgttgatcttttacttttggtaaacatttccattatattgtatttcagggattttgtacttttaagcattaggttcactaaataccacatctgcctatttactcaaattatttcctttacttatggctttttgcattatccagttttcctgacagcttgtatagattattgcctgaatttctctaaaacaaccaagctttcatttaagtgtcaaaaattattttatttctttacagtaattttaatttggatttcagtccttgcttatgttttgggagacccagccatctaccaaagcctgaaggcacagaatgcttattctcgtcactgtcctttctatgtcagcattcagagttactggctgtcatttttcatggtgatgattttatttgtagctttcataacctgttgggaagaagttactactttggtacaggctatcaggataacttcctatatgaatgaaactatcttatattttcctttttcatcccactccagttatactgtgagatctaaaaaaatattcttatccaagctcattgtctgttttctcagtacctggttaccatttgtactacttcaggtaatcattgttttacttaaagttcagattccagcatatattgagatgaatattccctggttatactttgtcaatagttttctcattgctacagtgtattggtttaattgtcacaagcttaatttaaaagacattggattacctttggatccatttgtcaactggaagtgctgcttcattccacttacaattcctaatcttgagcaaattgaaaagcctatatcaataatgatttgttaatattattaattaaaagttacagctgtcataagatcataattttatgaacagaaagaactcaggacatattaaaaaataaactgaactaaaacaacttttgccccctgactgatagcatttcagaatgtgtcttttgaagggctataccagttattaaatagtgttttattttaaaaacaaaataattccaagaagtttttatagttattcagggacactatattacaaatattactttgttattaacacaaaaagtgataagagttaacatttggctatactgatgtttgtgttactcaaaaaaactactggatgcaaactgttatgtaaatctgagatttcactgacaactttaagatatcaacctaaacatttttattaaatgttcaaatgtaagcaagaaaaaaaaaa

SEQ ID NO: 171 - DNA, 2285 bp, Homo sapiens
acccgcccccatctgcccaagataattttagtttccttgggcctggaatctggacacacagggctcccccccgcctctgacttctctgtccgaagtcgggacaccctcctaccacctgtagagaagcgggagtggatctgaaataaaatccaggaatctgggggttcctagacggagccagacttcggaacgggtgtcctgctactcctgctggggctcctccaggacaagggcacacaactggttccgttaagcccctctctcgctcagacgccatggagctggatctgtctccacctcatcttagcagctctccggaagacctttgcccagcccctgggacccctcctgggactccccggccccctgatacccctctgcctgaggaggtaaagaggtcccagcctctcctcatcccaaccaccggcaggaaacttcgagaggaggagaggcgtgccacctccctcccctctatccccaaccccttccctgagctctgcagtcctccctcacagagcccaattctcgggggcccctccagtgcaagggggctgctcccccgcgatgccagccgcccccatgtagtaaaggtgtacagtgaggatggggcctgcaggtctgtggaggtggcagcaggtgccacagctcgccacgtgtgtgaaatgctggtgcagcgagctcacgccttgagcgacgagacctgggggctggtggagtgccacccccacctagcactggagcggggtttggaggaccacgagtccgtggtggaagtgcaggctgcctggcccgtgggcggagatagccgcttcgtcttccggaaaaacttcgccaagtacgaactgttcaagagctccccacactccctgttcccagaaaaaatggtctccagctgtctcgatgcacacactggtatatcccatgaagacctcatccagaacttcctgaatgctggcagctttcctgagatccagggctttctgcagctgcggggttcaggacggaagctttggaaacgctttttctgcttcttgcgccgatctggcctctattactccaccaagggcacctctaaggatccgaggcacctgcagtacgtggcagatgtgaacgagtccaacgtgtacgtggtgacgcagggccgcaagctctacgggatgcccactgacttcggtttctgtgtcaagcccaacaagcttcgaaatggccacaaggggcttcggatcttctgcagtgaagatgagcagagccgcacctgctggctggctgccttccgcctcttcaagtacggggtgcagctgtacaagaattaccagcaggcacagtctcgccatctgcatccatcttgtttgggctccccacccttgagaagtgcctcagataataccctggtggccatggacttctctggccatgctgggcgtgtcattgagaacccccgggaggctctgagtgtggccctggaggaggcccaggcctggaggaagaagacaaaccaccgcctcagcctgcccatgccagcctccggcacgagcctcagtgcagccatccaccgcacccaactctggttccacgggcgcatttcccgtgaggagagccagcggcttattggacagcagggcttggtagacggcctgttcctggtccgggagagtcagcggaacccccagggctttgtcctctctttgtgccacctgcagaaagtgaagcattatctcatcctgccgagcgaggaggagggccgcctgtacttcagcatggatgatggccagacccgcttcactgacctgctgcagctcgtggagttccaccagctgaaccgcggcatcctgccgtgcttgctgcgccattgctgcacgcgggtggccctctgaccaggccgtggactggctcatgcctcagcccgccttcaggctgcccgccgcccctccacccatccagtggactctggggcgcggccacaggggacgggatgaggagcgggagggttccgccactccagttttctcctctgcttctttgcctccctcagatagaaaacagcccccactccagtccactcctgacccctctcctcaagggaaggccttgggtggccccctctccttctcctagctctggaggtgctgctctagggcagggaattatgggagaagtgggggcagcccaggcggtttcacgccccacactttgtacagaccgagaggccagttgatctgctctgttttatactagtgacaataaagattattttttgatacaaaaaaaaaaaaaaaaaaaaaaaa

SEQ ID NO: 172 - DNA, 935 bp, Homo sapiens
agtcagaggtcgcgcaggcgctggtaccccgttggtccgcgcgttgctgcgttgtgaggggtgtcagctcagtgcatcccaggcagctcttagtgtggagcagtgaactgtgtgtggttccttctacttggggatcatgcagagagcttcacgtctgaagagagagctgcacatgttagccacagagccacccccaggcatcacatgttggcaagataaagaccaaatggatgacctgcgagctcaaatattaggtggagccaacacaccttatgagaaaggtgtttttaagctagaagttatcattcctgagaggtacccatttgaacctcctcagatccgatttctcactccaatttatcatccaaacattgattctgctggaaggatttgtctggatgttctcaaattgccaccaaaaggtgcttggagaccatccctcaacatcgcaactgtgttgacctctattcagctgctcatgtcagaacccaaccctgatgacccgctcatggctgacatatcctcagaatttaaatataataagccagccttcctcaagaatgccagacagtggacagagaagcatgcaagacagaaacaaaaggctgatgaggaagagatgcttgataatctaccagaggctggtgactccagagtacacaactcaacacagaaaaggaaggccagtcagctagtaggcatagaaaagaaatttcatcctgatgtttaggggacttgtcctggttcatcttagttaatgtgttctttgccaaggtgatctaagttgcctaccttgaatttttttttaaatatatttgatgacataatttttgtgtagtttatttatcttgtacatatgtattttgaaatcttttaaacctgaaaaataaatagtcatttaatgttgaaaaaaaaaaaaaaaaaaaaaaaaaaaa

SEQ ID NO: 173 - DNA, 2896 bp, Homo sapiens
acgcttgcgcgcgggatttaaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaaaaaaaaaaaaaaaaa

SEQ ID NO: 174 - DNA, 2209 bp, Homo sapiens
actgcgcgcgtcgtgcgtaatgacgtcagcgccggcggagaatttcaaattcgaacggctttggcgggccgaggaaggacctggtgttttgatgaccgctgtcctgtctagcagatacttgcacggtttacagaaattcggtccctgggtcgtgtcaggaaactggaaaaaaggtcataagcatgaagcgcagttcagtttccagcggtggtgctggccgcctctccatgcaggagttaagatcccaggatgtaaataaacaaggcctctatacccctcaaaccaaagagaaaccaacctttggaaagttgagtataaacaaaccgacatctgaaagaaaagtctcgctatttggcaaaagaactagtggacatggatcccggaatagtcaacttggtatattttccagttctgagaaaatcaaggacccgagaccacttaatgacaaagcattcattcagcagtgtattcgacaactctgtgagtttcttacagaaaatggttatgcacataatgtgtccatgaaatctctacaagctccctctgttaaagacttcctgaagatcttcacatttctttatggcttcctgtgcccctcatacgaacttcctgacacaaagtttgaagaagaggttccaagaatctttaaagaccttgggtatccttttgcactatccaaaagctccatgtacacagtgggggctcctcatacatggcctcacattgtggcagccttagtttggctaatagactgcatcaagatacatactgccatgaaagaaagctcacctttatttgatgatgggcagccttggggagaagaaactgaagatggaattatgcataataagttgtttttggactacaccataaaatgctatgagagttttatgagtggtgccgacagctttgatgagatgaatgcagagctgcagtcaaaactgaaggatttatttaatgtggatgcttttaagctggaatcattagaagcaaaaaacagagcattgaatgaacagattgcaagattggaacaagaaagagaaaaagaaccgaatcgtctagagtcgttgagaaaactgaaggcttccttacaaggagatgttcaaaagtatcaggcatacatgagcaatttggagtctcattcagccattcttgaccagaaattaaatggtctcaatgaggaaattgctagagtagaactagaatgtgaaacaataaaacaggagaacactcgactacagaatatcattgacaaccagaagtactcagttgcagacattgagcgaataaatcatgaaagaaatgaattgcagcagactattaataaattaaccaaggacctggaagctgaacaacagaagttgtggaatgaggagttaaaatatgccagaggcaaagaagcgattgaaacacaattagcagagtatcacaaattggctagaaaattaaaacttattcctaaaggtgctgagaattccaaaggttatgactttgaaattaagtttaatcccgaggctggtgccaactgccttgtcaaatacagggctcaagtttatgtacctcttaaggaactcctgaatgaaactgaagaagaaattaataaagccctaaataaaaaaatgggtttggaggatactttagaacaattgaatgcaatgataacagaaagcaagagaagtgtgagaactctgaaagaagaagttcaaaagctggatgatctttaccaacaaaaaattaaggaagcagaggaagaggatgaaaaatgtgccagtgagcttgagtccttggagaaacacaagcacctgctagaaagtactgttaaccaggggctcagtgaagctatgaatgaattagatgctgttcagcgggaataccaactagttgtgcaaaccacgactgaagaaagacgaaaagtgggaaataacttgcaacgtctgttagagatggttgctacacatgttgggtctgtagagaaacatcttgaggagcagattgctaaagttgatagagaatatgaagaatgcatgtcagaagatctctcggaaaatattaaagagattagagataagtatgagaagaaagctactctaattaagtcttctgaagaatgaagataaaatgttgatcatgtatatatatccatagtgaataaaattgtctcagtaaagtgtaaaaaaaaaaaaaaaaaaaaaaaaaaaaaaaaaaaaaa

SEQ ID NO: 175 - DNA, 1740 bp, Homo sapiens
ctccctcctctgcaccatgactacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctatgggttggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaacgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 176 - DNA, 2895 bp, Homo sapiens
ctcttttgcaggggccgttcctcggggcatgacgctggctcctgcacagatcctgctcctctgtggccttcctgggctgccctcccctcctccgggactgctctggactgacactgctcaggttcggattccctcaaagactttgggagacaagacttggtcccccttttacaaacaagggaacggaggctctagaactgacttcctgaaaggcttggatccaaagctccctcagttcagcggccacgtctatttccctcagacacagggatccttgaacctgtgggctgtatctccccgcggacttggaagaatcccaagagagtggggctcccacaggctggagtgcaatggtgtgatctcggctcactgcaacctccacctcccaggttcaagctattctcctgcctcagcctcctgagtagctgggattacagatcctggtggctgtggtcggtaattccagcttcgtgctggctacaggtggatgatgcccacctggctgccgatgacctctgcaccaagtgaggctgggtctctggagctgccccaggggctggacaagctgaccctggccggggccaacctggagatgcagattgagaacctcaaggaggacctggtctacctgaagaagaaccacaagcaggaaatgaacgtcctttgaggtcaggtggatgaggatgtcagtgtgaagatggacactgtgcctggagtgaacctgagctgcatcctgaatgagatgcgtgaccaggacaagacattggtggagaagagctgcaaggatgccgagggctggttcttcagcatggtgggtggccgtgcgtaagcaggtgtgtacacgtgtgggcacatgtgctgcatgctggtgcagctggagcactggcagatccacaggctgtcccagttggaaggacttttggaaaccagttggaccagcccctcatgttttagatgtaaaacgtgaggctcagagaggactcaagctcacacagcccttcactgtggcctgcaaaatagatccaggtctctacaagtctggtcttgggtttccaccacagctgtttacaggatgtgcgtatttgaatacatatgtatacccttggcaagcacaggctgagtatctccggtatcctagggacagcaacaggcgcaaaagaataacacccagtgcctgtctttgaggtgctgcagttcagtaggaaaaagaaatgcaaatgaccgcagagcaggctgaattcctccaagttccaatgtgggtgcagaggctctctgtgtgcagaaagaggggctgaactgcgaggtggccaccaacacagaggccctgcagagtggctggatagagatatggagctctacgtctctgtgcagaacctgagccgtcccagctcagcaagaaagcatcgctggagggcagcctggtggagatggaggtgtgttacaggaccctgccggcccagctgcaggggcttaacagaagcatggagcagcagctgtgcgagctctgctgcgacacggagcaccaggaccacaagcacaggtccttctggacgtgaagacgtggctggagcaggagatcgccacctaccgccgcttgctggaggttgaggacgcccagaggtgatactgacgatgcaggctggagtctggctgaggagccttgaatgccaagttaaagcgtctggactagatcacgtaggcaatggggagccatggagggatttggagcaggagagtgaaatgaacatcaagagattttagaacattcactctggctgcagagggagaaatggatcagaggggtcagggcggggccagagagatgtgtcagggggctggagcagggagtctggccagagaagtcccgtgcggtggtgggtagtggggcaggggaaggaaggtggtgcacgcagaagagaggttatagctcaaaacagcgggactggatgcctggatctcggggtaagcatggctcacagtcaggactcagtaagtgtcgggagaacacatgaaggagcaggcattgatggccctgggtttctggttctgatgactgtgtgagtggtgaagagcaaggtgggtggtggttgggtttgcagttgggaagggtgatcaggccttcagctgagagtgtcccggagtctccatgcttagtcacacgttgcagctttttgctccccggaaatggtgaagtccatctatagtctaacaacagtctctcctgctttaattgggtctatttgttgggccctctgggttatggaaaaaccacttgctcagcttctccttgtaaattcctggtgagtagccacagagtgccgccagacctactgctgtgctgtttctttttcttcttcctgctgtgctgaacccctgccctttcattcttgggcctgcgctaatttctgtgcattcccaactgtgatttttcaccaatttaggggaacctcctctgccagggcctacttctccccagcagtgcttgcaggtgcctgggctggctggcatccctgggctgatgggtgcttctctccctgcaggctggccactcagtactccttgtccctggcctcgcagcccacccgggaagccacagtgaccagccaccaggtgtgccatcgtggaggaagtccaggttggagaggtggtcttcttctgtgagcaggtccacttctccacccactgagacccctttctgtctgcgacagccccacctcgagggccacggcacagccatcagctccagctcccagcatgctactgccacgccccgagtgtccgtctgggccccggtgcatggcctgttgtctttctgtatctactttctgcagcccctcactgaggaggcctcctgggtttgtccagtgcctactattaaagctttgctccaagttc

SEQ ID NO: 177 - DNA, 2529 bp, Homo sapiens
gcatcctttttgggctgctcacagcccccagcctctatggtgaagacatacttgctagcagcgtcaccaacttgctgccaagagatcagtgctgcaaggcaaggttatttctaactgagcagagcctgccaggaagaaagcgtttgcaccccacaccactgtgcaggtgtgaccggtgagctcacagctgccccccaggcatgcccagcccacttaatcattcacagctcgacagctctctcgcccagcccagttctggaagggataaaaagggggcatcaccgttcctgggtaacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagaggaggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatcacagagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatat

SEQ ID NO: 178 - DNA, 6816 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 179 - DNA, 2372 bp, Homo sapiens
gcaccgcgcgagcttggctgcttctggggcctgtgtggccctgtgtgtcggaaagatggagcaagaagccgagcccgaggggcggccgcgacccctctgaccgagatcctgctgctttcgcagccaggagcaccgtccctccccggattagtgcgtacgagcgcccagtgccctggcccggagagtggaatgatccccgaggcccagggcgtcgtgcttccgcagtagtcagtccccgtgaaggaaactggggagtcttgagggacccccgactccaagcgcgaaaaccccggatggtgaggagcaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctagttgacctgtctataagagaattatatatttctaactatataaccctaggaatttagacaacctgaaatttattcacatatatcaaagtgagaaaatgcctcaattcacatagatttcttctctttagtataattgacctactttggtagtggaatagtgaatacttactataatttgacttgaatatgtagctcatcctttacaccaactcctaattttaaataatttctactctgtcttaaatgagaagtacttggttttttttttcttaaatatgtatatgacatttaaatgtaacttattattttttttgagaccgagtcttgctctgttacccaggctggagtgcagtgggtgatcttggctcactgcaagctctgccctccccgggttcgcaccattctcctgcctcagcctcccaattagcttggcctacagtcatctgccaccacacctggctaattttttgtacttttagtagagacagggtttcaccgtgttagccaggatggtctcgatctcctgacctcgtgatccgcccacctcggcctcccaaagtgctgggattacaggcatgagccaccg

SEQ ID NO: 180 - DNA, 2486 bp, Homo sapiens
gagatttgattcccttggcgggcggaagcggccacaacccggcgatcgaaaagattcttaggaacgccgtaccagccgcgtctctcaggacagcaggcccctgtccttctgtcgggcgccgctcagccgtgccctccgcccctcaggttctttttctaattccaaataaacttgcaagaggactatgaaagattatgatgaacttctcaaatattatgaattacatgaaactattgggacaggtggctttgcaaaggtcaaacttgcctgccatatccttactggagagatggtagctataaaaatcatggataaaaacacactagggagtgatttgccccggatcaaaacggagattgaggccttgaagaacctgagacatcagcatatatgtcaactctaccatgtgctagagacagccaacaaaatattcatggttcttgagtactgccctggaggagagctgtttgactatataatttcccaggatcgcctgtcagaagaggagacccgggttgtcttccgtcagatagtatctgctgttgcttatgtgcacagccagggctatgctcacagggacctcaagccagaaaatttgctgtttgatgaatatcataaattaaagctgattgactttggtctctgtgcaaaacccaagggtaacaaggattaccatctacagacatgctgtgggagtctggcttatgcagcacctgagttaatacaaggcaaatcatatcttggatcagaggcagatgtttggagcatgggcatactgttatatgttcttatgtgtggatttctaccatttgatgatgataatgtaatggctttatacaagaagattatgagaggaaaatatgatgttcccaagtggctctctcccagtagcattctgcttcttcaacaaatgctgcaggtggacccaaagaaacggatttctatgaaaaatctattgaaccatccctggatcatgcaagattacaactatcctgttgagtggcaaagcaagaatccttttattcacctcgatgatgattgcgtaacagaactttctgtacatcacagaaacaacaggcaaacaatggaggatttaatttcactgtggcagtatgatcacctcacggctacctatcttctgcttctagccaagaaggctcggggaaaaccagttcgtttaaggctttcttctttctcctgtggacaagccagtgctaccccattcacagacatcaagtcaaataattggagtctggaagatgtgaccgcaagtgataaaaattatgtggcgggattaatagactatgattggtgtgaagatgatttatcaacaggtgctgctactccccgaacatcacagtttaccaagtactggacagaatcaaatggggtggaatctaaatcattaactccagccttatgcagaacacctgcaaataaattaaagaacaaagaaaatgtatatactcctaagtctgctgtaaagaatgaagagtactttatgtttcctgagccaaagactccagttaataagaaccagcataagagagaaatactcactacgccaaatcgttacactacaccctcaaaagctagaaaccagtgcctgaaagaaactccaattaaaataccagtaaattcaacaggaacagacaagttaatgacaggtgtcattagccctgagaggcggtgccgctcagtggaattggatctcaaccaagcacatatggaggagactccaaaaagaaagggagccaaagtgtttgggagccttgaaagggggttggataaggttatcactgtgctcaccaggagcaaaaggaagggttctgccagagacgggcccagaagactaaagcttcactataacgtgactacaactagattagtgaatccagatcaactgttgaatgaaataatgtctattcttccaaagaagcatgttgactttgtacaaaagggttatacactgaagtgtcaaacacagtcagattttgggaaagtgacaatgcaatttgaattagaagtgtgccagcttcaaaaacccgatgtggtgggtatcaggaggcagcggcttaagggcgatgcctgggtttacaaaagattagtggaagacatcctatctagctgcaaggtataattgatggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtcttttttaatcatgtggttttgtatattaataattgttgactttcttagattcacttccatatgtgaatgtaagctcttaactatgtctctttgtaatgtgtaatttctttctgaaataaaaccatttgtgaatatag

SEQ ID NO: 181 - DNA, 1140 bp, Homo sapiens
  misc_feature (1140)..(1140): n is a, c, g, or t
gcagcggaggagcccagtccacgatggcccggtccctggtgtgccttggtgtcatcatcttgctgtctgccttctccggacctggtgtcaggggtggtcctatgcccaagctggctgaccggaagctgtgtgcggaccaggagtgcagccaccctatctccatggctgtggcccttcaggactacatggcccccgactgccgattcctgaccattcaccggggccaagtggtgtatgtcttctccaagctgaagggccgtgggcggctcttctggggaggcagcgttcagggagattactatggagatctggctgctcgcctgggctatttccccagtagcattgtccgagaggaccagaccctgaaacctggcaaagtcgatgtgaagacagacaaatgggatttctactgccagtgagctcagcctaccgctggccctgccgtttcccctccttgggtttatgcaaatacaatcagcccagtgcaaaaaaaaaaaaaaaaaaaaaaaacttcggagaagagatagcaacaaaaggccgcttgtgtgaaggcgccaaaagttttcgcccaagagaccttcggcctcccccagggcgcgcgcaaaggcgccttgttttgacaacctcttggacaaccggaggggctaccgcccggagacccctgtggtggaccccccgggcaacccggtgtgacagggtactcacccccacggctttgtcgggggtcccaccaaaggccccaaagaggctctttcaaggcactattccttgttgtagaccttgtgtgtgccacaggcgccaaagaaacctcggggggctaacaaacgcacgtgcttggcagctccgagaaggctctctcccacccgaggggtggacgcaacagggggaatgggccatcatattgttgcccccggtgggcaccaactctttttcccccatagagaggccttagcacactatgtggggcacgttattgccgcctagagaaaccgagcgccagaaaatttcgaaggggggggcgcttctcatcattttgcgcaaaacccccttgtgggagtatgccccgaactcctctggaacacacaagcgacacttgcgcggggtctgcaaaaaacctcctgttgggaagccggcttcacn

SEQ ID NO: 182 - DNA, 12507 bp, Homo sapiens
taccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattaatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaaaaaaaaaaaa

SEQ ID NO: 183 - DNA, 3771 bp, Homo sapiens
gcgccgggacgtggccagttgcccgcctgccccggagagccaggcgctaaccagccgctctgcgccccgcgccctgcttgcccccattatccagccttgccccggcgccctgacctgacgccctggcctgacgccctgcttcgtcgcctcctttctctcccaggtgctggaccagggactgagcgtcccccggagagggtccggtgtgaccccgacaagaagcagaaatggggaagaaactggatctttccaagctcactgatgaagaggcccagcatgtcttggaagttgttcaacgagattttgacctccgaaggaaagaagaggaacggctagaggcgttgaagggcaagattaagaaggaaagctccaagagggagctgctttccgacactgcccatctgaacgagacccactgcgcccgctgcctgcagccctaccagctgcttgtgaatagcaaaaggcagtgcctggaatgtggcctcttcacctgcaaaagctgtggccgcgtccacccggaggagcagggctggatctgtgacccctgccatctggccagagtcgtgaagatcggctcactggagtggtactatgagcatgtgaaagcccgcttcaagaggttcggaagtgccaaggtcatccggtccctccacgggcggctgcagggtggagctgggcctgaactgatatctgaagagagaagtggagacagcgaccagacagatgaggatggagaacctggctcagaggcccaggcccaggcccagccctttggcagcaaaaaaaagcgcctcctctccgtccacgacttcgacttcgagggagactcagatgactccactcagcctcaaggtcactccctgcacctgtcctcagtccctgaggccagggacagcccacagtccctcacagatgagtcctgctcagagaaggcagcccctcacaaggctgagggcctggaggaggctgatactggggcctctgggtgccactcccatccggaagagcagccgaccagcatctcaccttccagacacggcgccctggctgagctctgcccgcctggaggctcccacaggatggccctggggactgctgctgcactcgggtcgaatgtcatcaggaatgagcagctgcccctgcagtacttggccgatgtggacacctctgatgaggaaagcatccgggctcacgtgatggcctcccaccattccaagcggagaggccgggcgtcttctgagagtcagatctttgagctgaataagcatatttcagctgtggaatgcctgctgacctacctggagaacacagttgtgcctcccttggccaagggtctaggtgctggagtgcgcacggaggccgatgtagaggaggaggccctgaggaggaagctggaggagctgaccagcaacgtcagtgaccaggagacctcgtccgaggaggaggaagccaaggacgaaaaggcagagcccaacagggacaaatcagttgggcctctcccccaggcggacccggaggtgggcacggctgcccatcaaaccaacagacaggaaaaaagcccccaggaccctggggaccccgtccagtacaacaggaccacagatgaggagctgtcagagctggaggacagagtggcagtgacggcctcagaagtccagcaggcagagagcgaggtttcagacattgaatccaggattgcagccctgagggccgcagggctcacggtgaagccctcgggaaagccccggaggaagtcaaacctcccgatatttctccctcgagtggctgggaaacttggcaagagaccagaggacccaaatgcagacccttcaagtgaggccaaggcaatggctgtgccctatcttctgagaagaaagttcagtaattccctgaaaagtcaaggtaaagatgatgattcttttgatcggaaatcagtgtaccgaggctcgctgacacagagaaaccccaacgcgaggaaaggaatggccagccacaccttcgcgaaacctgtggtggcccaccagtcctaacgggacaggacagagagacagagcagccctgcactgttttccctccaccacagccatcctgtccctcattggctctgtgctttccactatacacagtcaccgtcccaatgagaaacaagaaggagcaccctccacatggactcccacctgcaagtggacagcgacattcagtcctgcactgctcacctgggtttactgatgactcctggctgccccaccatcctctctgatctgtgagaaacagctaagctgctgtgacttccctttaggacaatgttgtgtaaatctttgaaggacacaccgaagacctttatactgtgatcttttacccctttcactcttggctttcttatgttgctttcatgaatggaatggaaaaaagatgactcagttaaggcaccagccatatgtgtattcttgatggtctatatcggggtgtgagcagatgtttgcgtatttcttgtgggtgtgactggatattagacatccggacaagtgactgaactaatgatctgctgaataatgaaggaggaatagacaccccagtccccaccctacgtgcacccgctctgcaagttcccatgtgatctgtagaccaggggaaattacactgcggtcaagggcagagcctgcacatgacagcaagtgagcatttgatagatgctcagatgctagtgcagagagcctgctgggagacgaagagacagcaggcagagctccagatgggcaaggaagaggcttggttctagcctggctctgcccctcactgcagtggatccagtggggcagaggacagagggtcacaaccaatgagggatgtctgccaaggatgggggtgcagaggccacaggagtcagcttgccactcgcccattggttacatagatgatctctcagacaggctgggactcagagttatttcctagtatcggtgtgccccatccagttttaagtggagccctccaagactctccagagctgcctttgaacatcctaacagtaatcacatctcaccctccctgaggttcactttagacaggacccaatggctgcactgcctttgtcagagggggtgctgagaggagtggcttcttttagaatcaaacagtagagacaagagtcaagccttgtgtcttcaagcattgaccaagttaagtgtttccttccctctctcaataagacacttccaggagctttccaatctctcacttaaaactaaggtttgaatctcaaagtgttgctgggaggctgatactcctgcaacttcaggagacctgtgagcacacattagcagctgtttctctgactccttgtggcatcagataaaaacgtgggagtttttccatataattcccagccttacttataaattctattctttgaaaaaattattcaggctaggtaaggtggctcatacctataatcccagccctttgagaggccaaggtgggagaattgcttgaggccaggagtttgagacctcctgggcaacatagtgagatcccatctctacaaaaaacaaaacaaaaaaattacccaagcatgatggtatatgcctgtagtcgtacctacttacttaggaggctgaggcaggaggatcacttgagccctggaggttggggctgcagtgagccatgatcgcatcactatactcgagcctgggcaacagagtgagaccttgtctcttaaaaaaattaataataaataaatgaaaataattcttcagaaaaaaaaaaaaaaaa

SEQ ID NO: 184 - DNA, 2240 bp, Homo sapiens
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtc

SEQ ID NO: 185 - DNA, 3167 bp, Homo sapiens
tagcagcacacaagggttcgtgtttgtggaaccaggtagcttccttcagagctgacatttgcccacagccagcctggcccagccccataccaccagccctggcgctctggggcgtgaggtgccttttctgcccccctgctctagggcaggtggaaatcacccatggtgggtctacatctgatagaagcatcttatagttctgcttctggaccagaccatcctgggtttttctctgttctgctgaagggttccctccacgtgtccatcacctcggtgaactcttgggagacctgggaagatgctggcctcacctctcgcctctcctttccctcattgtgctgccaccatccttctcacacaggctctccagggagagctgggcaggatgggatcttcctgggttcccaccttgctccgtgccccctctcactgttcctgaagtgtggccacggactgccttgttttctggaaagtcccaagtctggaccatgactgagcagcattctcggctatctgccacctgtctggggctcctggcccctcttagactcccctctcccttctgtttcccccgagcccctgacttggacctgcagggtggggagagggatgggacgagaacctgtgctggggccaaaggtcgcactgggggaaggtggagccagggcagcagagtgcctggcgtcggcccctatcctgtcactagttcccccgttctggcccctggcaggtttgtaaccccagatcagaagtactccatggacaacactccccacacgccaaccccgttcaagaacgccctggagaagtacggacccctgaagcccctggtacgtggtgtggtcactgccgtggatctctgcacagtgggatcccttcggttcatccaaccatgttcagtccacaggacccttccctctgaggtctcatttgattctttctcctgagaagatgcagagatcctgataatataaatggggaagctgaggctgctctttgtcacttcctccgactgctcctgagcacctgagtttgcaagcacgcgccggctggtgctagagacatggtggtatcccgtgacactcagcctcaggatgggggagactgatgtgaaatacaaataacttaaacactttcaggcaaagataagcactgggcctagttcagagaagtggcaaattgctactctggcctgtctctgaccaactcccagttctctacagagcacgggaaagcccctcggggacgtctttcctgcagtgtgcaggctgcccttctcccctgctcttcccagttgatgggatggttgtgttttctctatgaaaaaaggagttggcaccttgggctttctgaaacacacaggtgttttagaaatcagtggagggtgagagaaaggcatggttgtggaggcactggactgtgaacaaggtctgcagcgggtccccctgctgtctctctctactgcatggagcctcctatgaagcccaaggtggctgggggctgaggctcccttgggcctgccatggaactgattctgagtcaagcagactttccacggaccatgctacatgagccgaggtgaggcactagttagtgctcctttcctgttgcagtggagatttggctcctctgtactaaaatatctgcatgctctccaaacaggtgtgagggcaaatcacatgaccttggcagctgtaattaaagtttgtgggggcttttcggatgacttatgaggagtggctgtgattcgcacctttcactcttagtagcactcgccctcccctgttctctgttgcctgaagctggagaggtccttggaaccccgaggcctgagaaagggaaatgggtttgagagcccccattagtgtggaacaaagggttgagtgagcctgggctttgagctgtcggggtcctaattcagcagctgtgtgactgtgtgccaggctgttgatctctgagcttctgtttctacctgcttaaaatgacggttactgcacagggctgtgtgagggttacagtgcgtctctgggctgctcccagccatggcaggcccctgggaatcaaggtcatcagctgcttgtccaaggcagcagttagtggttgtgaatggtgcgtgtgagatctgcatcctggcgtcaggcctccttcctgccttacccaggacagcccagttgcagctgggttggtcccacagtcccacacacacacagcccgagtgtggtgcctcacgtgggctgccccgtgcctacccacagccacagaccccgcacctggaggaggacttgaaggaggtgctgcgttctgaggctggcatcgaactcatcatcgaggacgacatcaggcccgagaagcagaagaggaagcctgggctgcggcggagccccatcaagaaagtccggaagtctctggctcttgacattgtggatgaggatgtgaagctgatgatgtccacactgcccaagtctctatccttgccgacaactgccccttcaaactcttccagcctcaccctgtcaggtatcaaagaagacaacagcttgctcaaccagggcttcttgcaggccaagcccgagaaggcagcagtggcccagaagccccgaagccacttcacgacacctgcccctatgtccagtgcctggaagacggtggcctgcggggggaccagggaccagcttttcatgcaggagaaagcccggcagctcctgggccgcctgaagcccagccacacatctcggaccctcatcttgtcctgaggtgttgagggtgtcacgagcccattcacatgtttacaggggttgtgggggcagagggggtctgtgaatctgagagtcattcaggtgacctcctgcagggagccttctgccaccagcccctccccagactctcaggtggaggcaacagggccatgtgctgccctgttgccgagcccagctgtgggcggctcctggtgctaacaacaaagttccacttccaggtctgcctggttccccccccaaggccacagggagctccgtcagcttctcccaagcccacgtcaggcctggcctcatctcagaccctgcttaggatgggggatgtggccaggggtgctcctgtgctcaccctctcttggtgcatttttttggaagaataaaattgcctctctctttgaaaaaaaaaaaaaaaaa

SEQ ID NO: 186 - DNA, 2379 bp, Homo sapiens
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 187 - DNA, 1799 bp, Homo sapiens
gtgggaggattgcattcagtctagttcctggttgccggctgaaataacctgctctccaaaatgtccacaaaagtgacttaagtcaggttcccccaaaccagacaccaagacaagaatccatgtgtgtgtgactgaaggaagtgctgggagagccccagctgcagcctggatgtgaactgcaactccaaagtgtgtccagactcaaggcaagggcactaggctttccagacctcctactaagtcattgatccagcactgccctgccaggacataaatccctggcacctcttgctctctgcaaaggagggcaaagcagcttcaggagcccttgggagtcctccaaagagagtctagggtacaggtccgaaagtagaagaacacagaaggcaggccaggggcactgtgagatggtaaaagagatctgaagggatccagaattcaagccaggaagaagcagcaatctgtcttctggattaaaactgaagatcaacctactttcaacttactaagaaaggggatcatggacattgaagcatatcttgaaagaattggctataagaagtctaggaacaaattggacttggaaacattaactgatattcttcaacaccagatccgagctgttccctttgagaaccttaacatccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgtttacagcactccagccaaaaaatacagcactggcatgattcaccttctcctgcaggtgaccattgatggcaggaactacattgtcgatgctgggtttggacgctcataccagatgtggcagcctctggagttaatttctgggaaggatcagcctcaggtgccttgtgtcttccgtttgacggaagagaatggattctggtatctagaccaaatcagaagggaacagtacattccaaatgaagaatttcttcattctgatctcctagaagacagcaaataccgaaaaatctactcctttactcttaagcctcgaacaattgaagattttgagtctatgaatacatacctgcagacatctccatcatctgtgtttactagtaaatcattttgttccttgcagaccccagatggggttcactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgatagattttttactatttagaataaggagtaaaacaatcttgtctatttgtcatccagctcaccagttatcaactgacgacctatcatgtatcttctgtacccttaccttattttgaagaaaatcctagacatcaaatcatttcacctataaaaatgtcatcatatataattaaacagctttttaaagaaacataaccacaaaccttttcaaataataataataataataataataaatgtcttttaaagatggcctgtggttatcttggaaattggtgatttatgctagaaagcttttaatgttggtttattgttgaattcctagaaaagttttatgggtagatgagtaaataaaatattgtaaaaaaacttattgtctataaagtatattaaaacattgttggctaatataaaaaaaaaaaaaa

SEQ ID NO: 188 - DNA, 1644 bp, Homo sapiens
gcgcgcgggtttcgttgacccgcggcgttcacgggaattgttcgctttagtgccggcgccatggggtcggagctgatcgggcgcctagccccgcgcctgggcctcgccgagcccgacatgctgaggaaagcagaggagtacttgcgcctgtcccgggtgaagtgtgtcggcctctccgcacgcaccacggagaccagcagtgcagtcatgtgcctggaccttgcagcttcctggatgaagtgccccttggacagggcttatttaattaaactttctggtttgaacaaggagacatatcagagctgtcttaaatcttttgagtgtttactgggcctgaattcaaatattggaataagagacctagctgtacagtttagctgtatagaagcagtgaacatggcttcaaagatactaaaaagctatgagtccagtcttccccagacacagcaagtggatcttgacttatccaggccacttttcacttctgctgcactgctttcagcatgcaagattctaaagctgaaagtggataaaaacaaaatggtagccacatccggtgtaaaaaaagctatatttgatcgactgtgtaaacaactagagaagattggacagcaggtcgacagagaacctggagatgtagctactccaccacggaagagaaagaagatagtggttgaagccccagcaaaggaaatggagaaggtagaggagatgccacataaaccacagaaagatgaagatctgacacaggattatgaagaatggaaaagaaaaattttggaaaatgctgccagtgctcaaaaggctacagcagagtgatttcagcttccaaactggtatacattccaaactgatagtacattgccatctccaggaagacttgacggctttgggattttgtttaaacttttataataaggatcctaagactgttgcctttaaatagcaaagcagcctacctggaggctaagtctgggcagtgggctggcccctggtgtgagcattagaccagccacagtgcctgattggtatagccttatgtgctttcctacaaaatggaattggaggccgggcgcagtggctcacgcctgtaatcccagcactttgggaggccaaggtgggtggatcacctgaggtcaggagctcgagaccagcctggccaacatggtgaaaccccatctctactaaaaatacaaaaattagccaggtgtgatggtgcatgcctgtaatcccagctcctcagtaggctgagacaggagcatcacttgaacgtgggaggcagaggttgcagtgagccgagattgcaccaccgcactccagcctgggtgacagagcgagacttatctcataaataaatagatagatactccagcctgggtgacagagcgagacttatagatagatagatagatagatggatagatagatagatagatagatagatagataaacggaattggagccattttgctttaagtgaatggcagtcccttgtcttattcagaatataaaattcagtctgaatggcatcttacagattttacttcaatttttgtgtacggtattttttatttgactaaatcaatatattgtacagcctaagttaataaatgttatttatatatgcaaaaaaaaaaaaaaaaa

SEQ ID NO: 189 - DNA, 13037 bp, Homo sapiens
agtccacagctgtcactaatcggggtaagccttgttgtatttgtgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatccctccggtccagccacattcaacacccactttctcctccctctgcccctatattcccgaaaccccctcctccttcccttttccctcctcctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctgaaagccttacatttataacatcatagtgtgtaaatttaaaagaaaaattgtgaggttctaattattttcttttataaagtataattagaatgtttaactgttttgtttacccatattttcttgaagaatttacaagattgaaaaagtactaaaattgttaaagtaaactatcttatccatattatttcataccatgtaggtgaggatttttaacttttgcatctaacaaatcatcgacttaagagaaaaaatcttacatgtaataacacaaagctattatatgttatttctaggtaactccctttgtgtcaattatatttccaaaaatgaacctttaaaatggtatgcaaaattttgtctatatatatttgtgtgaggaggaaattcataactttcctcagattttcaaaagtatttttaatgcaaaaaatgtagaaagagtttaaaaccactaaaatagattgatgttcttcaaactaggcaaaacaactcatatgttaagaccattttccagattggaaacacaaatctcttaggaagttaataagtagattcatatcattatgcaaatagtattgtgggttttgtaggtttttaaaataaccttttttggggagagaattgtcctctaatgaggtattgcgagtggacataagaaatcagaagattatggcctaactgtactccttaccaactgtggcatgctgaaagttagtcactcttactgattctcaattctctcacctttgaaagtagtaaaatatctttcctgccaattgctcctttgggtcagagcttattaacatcttttcaaatcaaaggaaagaagaaagggagaggaggaggagggaggtatcaattcacatacctttctcctctttatcctccactatcatgaattcatattatgtttcagccatgcaaatctttttaccatgaaatttcttccagaattttccccctttgacacaaattccatgcatgtttcaaccttcgagactcagccaaatgtcatttctgtaaaatcttccctgagtcttccaagcagtaatttgccttctcctagagtttacctgccattttgtgcacatttgagttacagtagcatgttattttacaattgtgactctcctgggagtctgggagccatataaagtggtcaatagtgtttgctgactgagagttgaatgacattttctctctgtcttggtattactgtagatttcgatcattctttggttacatttctgcatatttctgtacccatgactttatcactttcttctcccatgctttatctccatcaattatcttcattacttttaaattttccacctttgcttcctactttgtgagatctctccctttactgactataacatagaagaatagaagtgtattttatgtgtcttaaggacaatactttagattccttgttctaagtttttaaactgaatgaatggaatattatttctctccctaagcaaaattccacaaaacaattatttcttatgtttatgtagccttaaattgttttgtactgtaaacctcagcataaaaactttcttcatttctaatttcattcaacaaatattgattgaatacctggtattagcacaagaaaaatgtgctaataagccttatgagaatttggagctgaagaaagacatataactcaggaaagttacagtccagtagtaggtataaattacagtgcctgataaataggcattttaatatttgtacactcaacgtatactaggtaggtgcaaaacatttacatataattttactgatacccatgcagcacaaaggtactaactttaaatattaaataacacctttatgtgtcagtaattcatttgcattaaatcttattgaaaaggctttcaatatattttccccacaaatgtcatcccaagaaaaaagtatttttaacatctcccaaatataatagttacaggaaatctacctctgtgagagtgacacctctcagaatgaactgtgtgacacaagaaaatgaatgtaggtctatccaaaaaaaaccccaagaaacaaaaacaatattattagccctttatgcttaagtgatggactcagggaacagttgatgttgtgatcattttattatctgattcttgttactttgaattaaaccaatattttgatgatataaatcatttccaccagcatatatttaatttccataataactttaaaattttctaatttcactcaactatgagggaatagaatgtggtggccacaggtttggcttttgttaaaatgtttgatatcttcgatgttgatctctgtctgcaatgtagatgtctaaacactaggatttaatatttaaggctaagctttaaaaataaagtacctttttaaaaagaatatggcttcaccaaatggaaaatacctaatttctaaatctttttctctacaaagtcctatctactaatgtctccattactatttagtcatcataaccattatcttcattttacatgtcgtgttctttctggtagctctaaaatgacactaaatcataagaagacaggttacatatcaggaaatacttgaaggttactgaaatagattcttgagttaatgaaaatattttctgtaaaaaggtttgaaaagccatttgagtctaaagcattatacctccattatcagtagttatgtgacaattgtgtgtgtgtttaatgtttaaagatgtggcactttttaataaggcaatgctatgctattttttcccatttaacattaagataatttattgctatacagatgatatggaaatatgatgaacaatattttttttgccaaaactatgccttgtaagtagccatggaatgtcaacctgtaacttaaattatccacagatagtcatgtgtttgatgatgggcactgtggagataactgacataggactgtgccccccttctctgccacttactagctggatgagattaagcaagtcatttaactgctctgattaaacctgcctttcccaagtgctttgtaatgaatagaaatggaaaccaaaaaaaacgtatacaggccttcagaaatagtaattgctactattttgttttcattaagccatagttctggctataattttatcaaactcaccagctatattctacagtgaaagcaggattctagaaagtctcactgttttatttatgtcaccatgtgctatgatatatttggttgaattcatttgaaattagggctggaagtattcaagtaatttcttctgctgaaaaaatacagtgttttgagtttagggcctgttttatcaaagttctaaagagcctatcactcttccattgtagacattttaaaataatgacactgattttaacatttttaagtgtctttttagaacagagagcctgactagaacacagcccctccaaaaacccatgctcaaattatttttactatggcagcaattccacaaaagggaacaatgggtttagaaattacaatgaagtcatcaacccaaaaaacatccctatccctaagaaggttatgatataaaatgcccacaagaaatctatgtctgctttaatctgtcttttattgctttggaaggatggctattacatttttagtttttgctgtgaatacctgagcagtttctctcatccatacttatccttcacacatcagaagtcaggatagaatatgaatcattttaaaaacttttacaactccagagccatgtgcataagaagcattcaaaacttgccaaaacatacattttttttcaaatttaaagatactctatttttgtattcaatagctcaacaactgtggtccccactgataaagtgaagtggacaaggagacaagtaatggcataagtttgtttttcccaaagtatgcctgttcaatagccattggatgtgggaaatttctacatctcttaaaattttacagaaaatacatagccagatagtctagcaaaagttcaccaagtcctaaattgcttatccttacttcactaagtcatgaaatcattttaatgaaaagaacatcacctaggttttgtggtttctttttttcttattcatggctgagtgaaaacaacaatctctgtttctccctagcatctgtggactatttaatgtaccattattccacactctatggtccttactaaatacaaaattgaacaaaaagcagtaaaacaactgactcttcacccatattataaaatataatccaagccagattagtcaacatccataagatgaatccaagctgaactgggcctagattattgagttcaggttggatcacatccctatttattaataaacttaggaaagaaggccttacagaccatcagttagctggagctaatagaacctacacttctaaagttcggcctagaatcaatgtggccttaaaagctgaaaagaagcaggaaagaacagttttcttcaataatttgtccaccctgtcactggagaaaatttaagaatttgggggtgttggtagtaagttaaacacagcagctgttcatggcagaaattattcaatacataccttctctgaatatcctataaccaaagcaaagaaaaacaccaaggggtttgttctcctccttggagttgacctcattccaaggcagagctcaggtcacaggcacaggggctgcgcccaagcttgtccgcagccttatgcagctgtggagtctggaagactgttgcaggactgctggcctagtcccagaatgtcagcctcattttcgatttactggctcttgttgctgtatgtcatgctgaccttattgttaaacacaggtttgtttgctttttttccactcatggagacatgggagaggcattatttttaagctggttgaaagctttaaccgataaagcatttttagagaaatgtgaatcaggcagctaagaaagcatactctgtccattacggtaaagaaaatgcacagattattaactctgcagtgtggcattagtgtcctggtcaatattcggatagatatgaataaaatatttaaatggtattgtaaatagttttcaggacatatgctatagcttatttttattatcttttgaaattgctcttaatacatcaaatcctgatgtattcaatttatcagatataaattattctaaatgaagcccagttaaatgtttttgtcttgtcagttatatgttaagtttctgatctctttgtctatgacgtttactaatctgcatttttactgttatgaattattttagacagcagtggtttcaagctttttgccactaaaaataccttttattttctcctcccccagaaaagtctataccttgaagtatctatccaccaaactgtacttctattaagaaatagttattgtgttttcttaatgttttgttattcaaagacatatcaatgaaagctgctgagcagcatgaataacaattatatccacacagatttgatatattttgtgcagccttaacttgatagtataaaatgtcattgctttttaaataatagttagtcaatggacttctatcatagctttcctaaactaggttaagatccagagctttggggtcataatatattacatacaattaagttatctttttctaagggctttaaaattcatgagaataaccaaaaaaggtatgtggagagttaatacaaacataccatattcttgttgaaacagagatgtggctctgcttgttctccataaggtagaaatactttccagaatttgcctaaactagtaagccctgaatttgctatgattagggataggaagagattttcacatggcagactttagaattcttcactttagccagtaaagtatctccttttgatcttagtattctgtgtattttaacttttctgagttgtgcatgtttataagaaaaatcagcacaaagggtttaagttaaagcctttttactgaaatttgaaagaaacagaagaaaatatcaaagttctttgtattttgagaggattaaatatgatttacaaaagttacatggagggctctctaaaacattaaattaattattttttgttgaaaagtcttactttaggcatcattttattcctcagcaactagctgtgaagcctttactgtgctgtatgccagtcactctgctagattgtggagattaccagtgttcccgtcttctccgagcttagagttggatggggaataaagacaggtaaacagatagctacaatattgtactgtgaatgcttatgctggaggaagtacagggaactattggagcacctaagaggagcacctaccttgaatttaggggttagcagaggcatcctgaaaaaagtcaaagctaagccacaatctataagcagtttaggaattagcagaacgtgcgtggtgaggagatgccaaaggcaagaagagaagagtattccaaacaggagggattccaaagagagaagagtatcccaaacaacatttgcacaaacctgatggggagagagaatgtggggtggggatggatgatgagactgaagaagaaagccaggtctagataatcagtggccttgtacaccatgttaaagagtgtagacttgattctgttgtaaacaggaaagcagcacaattcatatgaatattttagaagactcccactggaatatggagaataaagttggagatgactaatcctggaagcagggagaacatttttgaggaagttgcactattttggtgaaaatgatgatcataaacatgaagaattgtaggtgatcatgacctcctctctaattttccagaagggttttggaagatataacataggaacattgacaggactgacgaaaggagatgaaatacaccatataaattgtcaaacacaaggccagatgtctaattattttgcttatgtgttgaaattacaaatttttcatcaggaaaccaaaaactacaaaacttagttttcccaagtcccagaattctatctgtccaaacaatctgtaccactccacctatatccctacctttgcatgtctgtccaacctcaaagtccaggtctatacacacgggtaagactagagcagttcaagtttcagaaaatgagaaagaggaactgagttgtgctgaacccatacaaaataaacacattctttgtatagattcttggaacctcgagaggaattcacctaactcataggtatttgatggtatgaatccatggctgggctcggcttttaaaaagccttatctgggattccttctatggaaccaagttccatcaaagcccatttaaaagcctacattaaaaacaaaattcttgctgcattgtatacaaataatgatgtcatgatcaaataatcagatgccattatcaagtggaattacaaaatggtatacccactccaaaaaaaaaaaaaaagctaaattctcagtagaacattgtgacttcatgagccctccacagccttggagctgaggagggagcactggtgagcagtaggttgaagagaaaacttggcgcttaataatctatccatgttttttcatctaaaagagccttctttttggattaccttattcaatttccatcaaggaaattgttagttccactaaccagacagcagctgggaaggcagaagcttactgtatgtacatggtagctgtgggaaggaggtttctttctccaggtcctcactggccatacaccagtcccttgttagttatgcctggtcatagacccccgttgctatcatctcatatttaagtctttggcttgtgaatttatctattctttcagcttcagcactgcagagtgctgggactttgctaacttccatttcttgctggcttagcacattcctcataggcccagctcttttctcatctggccctgctgtggagtcaccttgccccttcaggagagccatggcttaccactgcctgctaagcctccactcagctgccaccacactaaatccaagcttctctaagatgttgcagactttacaggcaagcataaaaggcttgatcttcctggacttccctttacttgtctgaatctcacctccttcaactttcagtctcagaatgtaggcatttgtcctctttgccctacatcttccttcttctgaatcatgaaagcctctcacttcctcttgctatgtgctggaggcttctgtcaggttttagaatgagttctcatctagtcctagtagcttttgatgcttaagtccaccttttaaggatacctttgagatttagaccatgtttttcgcttgagaaagccctaatctccagacttgcctttctgtggatttcaaagaccaactgaggaagtcaaaagctgaatgttgactttctttgaacatttccgctataacaattccaattctcctcagagcaatatgcctgcctccaactgaccaggagaaaggtccagtgccaaagagaaaaacacaaagattaattatttcagttgagcacatactttcaaagtggtttgggtattcatatgaggttttctgtcaagagggtgagactcttcatctatccatgtgtgcctgacagttctcctggcactggctggtaacagatgcaaaactgtaaaaattaagtgatcatgtattttaacgatatcatcacatacttattttctatgtaatgttttaaatttcccctaacatactttgactgttttgcacatggtagatattcacatttttttgtgttgaagttgatgcaatcttcaaagttatctaccccgttgcttattagtaaaactagtgttaatacttggcaagagatgcagggaatctttctcatgactcacgccctatttagttattaatgctactaccctattttgagtaagtagtaggtccctaagtacattgtccagagttatacttttaaagatatttagccccatatacttcttgaatctaaagtcatacaccttgctcctcatttctgagtgggaaagacatttgagagtatgttgacaattgttctgaaggtttttgccaagaaggtgaaactgtcctttcatctgtgtatgcctggggctgggtccctggcagtgatggggtgacaatgcaaagctgtaaaaactaggtgctagtgggcacctaatatcatcatcatatacttattttcaagctaatatgcaaaatcccatctctgtttttaaactaagtgtagatttcagagaaaatattttgtggttcacataagaaaacagtctactcagcttgacaagtgttttatgttaaattggctggtggtttgaaatgaatcatcttcacataatgttttctttaaaaatattgtgaatttaactctaattcttgttattctgtgtgataataaagaataaactaatttcta

SEQ ID NO: 190 - DNA, 3180 bp, Homo sapiens
attctatgctgcagcctaagcatcattcctcttctcttcttagtggagataaaattacccactgctctccttacatttactttgtccatatttgctcctatgctctaggctcgtgcacaacaaacacagtgtgggcccttaccctagaagccaacttctcatgacctttctctatctccagaatccatgcagtgggaatgaaggtaaaagaaggttttcatgggatccagctgagagctctacggggaaaatggatctgaggagccatgtgctccatctcttttattttacaggtagagactaggggtatagagtgaggtgaattaccgcagtgacccacacattgttggcagacctaggattagaactctgtcttcctggttcccagcttggtgcttttgaaagcatacttgctgctttcttaccggcctggtgtctgccactttgggacagagtgtggacttgctcacctgccccatttcttagggattctcattctgtgtttgagcaagaatattcttattctggaaagaaccacataccacaggattctgggtgagcataaggaagattgtcttggggatctgacttagctcacgtatagtggctatgatgaattcagtgtcttattttttgcatatgtatatttttagtctaatattgcctgggtgtctgagcaagtctagatgaatttaattgctctcatttttcccctgcccctcttcctttggtctctcttttaggaaatgtttttctttcaacattcgtttcattcattatttactcattcggccaaccaacatttattgagtgccttccctgtatcagggacaggggcttacaaagtagaatttgatcccacctctgccctcagtagctcagtgtctaatggaggtagtgatgttcattaagcgtcgccagatactgtgctaggtgctgtgcctgttctctctcgcttgttcctcacacacttgagaaggccgaagctgattcatagcttggaaggcaggggccttggatttgaacccaggcctgaccaatggcagaacctatcagatgtgtggacagatgacattgcctttctttctttggatatatcaaaatcagccagcaggcaggaactcccattttgagcaagcaatgtgcaggaatgatagggtatacagagaggaacaggagatggcccctgacttccagcatgtgtctgatggacatccaggctgcaggcatcatggtgctgtctagagagatgagccaggtgcccagagcccatgggccaatgctgccctttcttgagcatgccaaacaaagcggttggtgtgttagaggcacagtctcctccactctaagtaaaaatcagcatgagtcctagcccacatttccctagtgagtacaccaaagatatctatgaactggcagtcatcagtgacttcctaaggttccggaaatgcatctcttactcaggagtaagcaatgatgtgcctgcggctttacgagttctcacagaatgactttctggacccaaatgttttttctgcttcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaacacccccaatgggaacagcctcagtgccgcagaactcacttgtggaatgatcatgtgcctggccaggcagattccccaggcgacggcttcgatgaaggacggcaaatgggagcggaagaagttcatgggaacagagctgaatggaaagaccctgggaattcttggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggcccttaccagtgccttctctccacacaccaagccttggattggtctggcagaagctctggggacactgatgcgagcctgggctgggtcccccaaagggaccatccaggtgataacacagggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtcaccacctcccacagccctgctgcaccaggggggcaaggcttcggggaatgcctcctggccgtggccctggcaggcgccccttaccaggctgtgggcttggtccaaggcactacacctgtactgcaggggctcaatggagctgtcttcaggccagaagtgcctctccgcagggacctgcccctgctcctattccggactcagacctctgaccctgcaatgctgcctaccatgattggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaaccttggagctcactggtccctgcctctggggcttttctgaagaaacccacccactgtgatcaatagggagagaaaatccacattcttgggctgaacgcgagcctctgacactgcttacactgcactctgaccctgtagtacagcaataaccgtctaataaagagcctaccccc

SEQ ID NO: 191 - DNA, 1301 bp, Homo sapiens
  misc_feature (1301)..(1301): n is a, c, g, or t
caaacaaaaacagccaagcttttctgccaaaaagatgactgagaagactgttaaagcaaaaagctctgttcctgcctcagatgatgcctatccagaaatagaaaaattctttcccttcaatcctctagactttgagagttttgacctgcctgaagagcaccagattgcgcacctccccttgagtggagtgcctctcatgatccttgacgaggagagagagcttgaaaagctgtttcagctgggccccccttcacctgtgaagatgccctctccaccatgggaatccaatctgttgcagtctccttcaagcattctgtcgaccctggatgttgaattgccacctgtttgctgtgacatagatatttaaatttcttagtgcttcagagtctgtgtgtatttgtattaataaagcattctttaacagaaaaaaaaaaaaaaaaaaaaaaaaaaaaaaaaaaaaaaaaaaaaaaaggggggggagacacaaaaagaattccccaagagggggccacaagataatcagaggatatcacacaagatctctcggcgcaccaacgacgggggccccaaataagggagagacccagaatcacaacagccaagacacggtggacacgacggaaacaaacacacagcccagacacgggggcaaacacgcgcgcacaccgcggacaccatgggacaaagcagacaccacccacaaaacaacaccgcggagggggaagaacaacaaaacaagtgcgcaaacagaacacaaccacagaaagagaaaaattaaaacggcccccaagacggcgacaacacaacaaaacaaccactacagagcgctcaacagccgagtaaaaacacaacaacggacaactaacacacaaaggaatgaaacaaagcggggccacacaccgacaccggaaatccggcgaacaactcacaccgagcgagggtcccagacaacaaatacacagacaacgaaaccgagaaacaagaccagcaagacgagcaggcaaaagacaaacaagacagaggagacgacgacgaacgcaaaggacaagaggacacaacgacgcgaggagcgagagcgagaggaagagacaacaaaaagacacaaaagaacaacaagcaagcagcgaagaacgacacacaaccacacgagacagcaggagcagaggcggagaaaacacaacgagcaagccaagaccaagagaggagaacaaaataaaaaaatacgagagcaggcggacgagagcacgagacgaacagacaaacgggaatcagaagcataacgatccgcgacgcgaacaacn

SEQ ID NO: 192 - DNA, 3203 bp, Homo sapiens
gtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgatttttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgtttgtactcacaaggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcatacttcagtcacccagtgtcttattctggcattgtctaaatctgagcattgtctagggggatcttaaactttagtaggaaaccatgagctgttaatacagtttccattcaaatattaatttcagaatgaaacataattttttttttttttttttgagatggagtctcgctctgttgcccaggctggagtgcagtggcgcgattttggctcactgtaacctccatctcctgggttcaagcaattctcctgtctcagcctccctagtagctgggactgcaggtatgtgctaccacacctggctaatttttgtatttttagtagagatggagtttcaccatattggtcaggctggtcttgaactcctgacctcaggtgatccacccacctcggcctcccaaagtgctgggattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagctcagaccatttcctaatcagttgaaagggaaacaagtatttcagtctcaaaattgaataatgcacaagtcttaagtgattaaaataaaactgttcttatgtcagttt

SEQ ID NO: 193 - DNA, 4482 bp, Homo sapiens
agcgggggcactccagccctgcagcctccggagtcagtgccgcgcgcccgccgccccgcgccttcctgctcgccgcacctccgggagccggggcgcacccagcccgcagcgccgcctccccgcccgcgccgcctccgaccgcaggccgagggccgccactggccggggggaccgggcagcagcttgcggccgcggagccgggcaacgctggggactgcgccttttgtccccggaggtccctggaagtttgcggcaggacgcgcgcggggaggcggcggaggcagccccgacgtcgcggagaacagggcgcagagccggcatgggcatcgggcgcagcgaggggggccgccgcggggcagccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtgattctcccgggggcagggtggggagggagcctcgggtggggtgggagcgggggggacagtgccccgggaacccggtgggtcacacacacgcactgcgcctgtcagtagtggacatttaatccagtcggcttgttcttgcagcattcccgctcccttccctccatagccacgctccaaaccccagggtagccatggccgggtaaagcaagggccatttagattaggaaggtttttaagatccgcaatgtggagcagcagccactgcacaggaggaggtgacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtaatagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcagtagttttcaaaacacatagttaaaaaagaaacaaatgaaaaaaattttagaacagtccagcaaattgctagtcagggtgaattgtgaaattgggtgaagagcttacgattctaatctcatgttttttccttttcacatttttaaaagaacaatgacaaacacccacttatttttcaaggttttaaaacagtctacattgagcatttgaaaggtgtgctagaacaaggtctcctgatccgtccgaggctgcttcccagaggagcagctctccccaggcatttgccaagggaggcggatttccctggtagtgtagctgtgtggctttccttcctgaagagtccgtggttgccctagaacctaacaccccctagcaaaactcacagagctttccgtttttttctttcctgtaaagaaacatttcctttgaacttgattgcctatggatcaaagaaattcagaacagcctgcctgtccccccgcactttttacatatatttgtttcatttctgcagatggaaagttgacatgggtggggtgtccccatccagcgagagagtttaaaaagcaaaacatctctgcagtttttcccaagtgccctgagatacttcccaaagcccttatgtttaatcagcgatgtatataagccagttcacttagacaactttacccttcttgtccaatgtacaggaagtagttctaaaaaaaatgcatattaatttcttcccccaaagccggattcttaattctctgcaacactttgaggacatttatgattgtccctctgggccaatgcttatacccagtgaggatgctgcagtgaggctgtaaagtggccccctgcggccctagcctgacccggaggaaaggatggtagattctgttaactcttgaagactccagtatgaaaatcagcatgcccgcctagttacctaccggagagttatcctgataaattaacctctcacagttagtgatcctgtccttttaacaccttttttgtggggttctctctgacctttcatcgtaaagtgctggggaccttaagtgatttgcctgtaattttggatgattaaaaaatgtgtatatatattagctaattagaaatattctacttctctgttgtcaaactgaaattcagagcaagttcctgagtgcgtggatctgggtcttagttctggttgattcactcaagagttcagtgctcatacgtatctgctcattttgacaaagtgcctcatgcaaccgggccctctctctgcggcagagtccttagtggaggggtttacctggaacattagtagttaccacagaatacggaagagcaggtgactgtgctgtgcagctctctaaatgggaattctcaggtaggaagcaacagcttcagaaagagctcaaaataaattggaaatgtgaatcgcagctgtgggttttaccaccgtctgtctcagagtcccaggaccttgagtgtcattagttactttattgaaggttttagacccatagcagctttgtctctgtcacatcagcaatttcagaaccaaaagggaggctctctgtaggcacagagctgcactatcacgagcctttgtttttctccacaaagtatctaacaaaaccaatgtgcagactgattggcctggtcattggtctccgagagaggaggtttgcctgtgatttcctaattatcgctagggccaaggtgggatttgtaaagctttacaataatcattctggatagagtcctgggaggtccttggcagaactcagttaaatctttgaagaatatttgtagttatcttagaagatagcatgggaggtgaggattccaaaaacattttatttttaaaatatcctgtgtaacacttggctcttggtacctgtgggttagcatcaagttctccccagggtagaattcaatcagagctccagtttgcatttggatgtgtaaattacagtaatcccatttcccaaacctaaaatctgtttttctcatcagactctgagtaactggttgctgtgtcataacttcatagatgcaggaggctcaggtgatctgtttgagcagagcaccctaggcagcctgcagggaataacatactggccgttctgacctgttgccagcagatacacaggacatggatgaaattcccgtttcctctagtttcttcctgtagtactcctcttttagatcctaagtctcttacaaaagctttgaatactgtgaaaatgttttacattccatttcatttgtgttgtttttttaactgcattttaccagatgttttgatgttatcgcttatgttaatagtaattcccgtacgtgttcattttattttcatgctttttcagccatgtatcaatattcacttgactaaaatcactcaattaatcaaaaaaaaaaaaaaaa

SEQ ID NO: 194 - DNA, 3637 bp, Homo sapiens
agtcctgggcgaagggggcggtggttccccgcggcgctgcgcgcggcggtaattagtgattgtcttccagcttcgcgaaggctaggggcgcggctgccgggtggctgcgcggcgctgcccccggaccgaggggcagccaacccaatgaaaccaccgcgtgttcgcgcctggtagagatttctcgaagacaccagtgggcccgttccgagccctctggaccgcccgtgtggaaccaaacctgcgcgcgtggccgggccgtgggacaacgaggccgcggagacgaaggcgcaatggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccatgatcatcactctcaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactctggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctagttaaggtttaaatgctagagtagcttaaaaagttgtcatagtttcagtaggtcatagggagatgagtttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtattaccagtttattatgtaaacaagagatttggcatgacatgttctgtatgtttcagggaaaaatgtctttaatgctttttcaagaactaacacagttattcctatactggattttaggtctctgaagaactgctggtgtttaggaataagaatgtgcatgaagcctaaaataccaagaaagcttatactgaatttaagcaaagaaataaaggagaaaagagaagaatctgagaattggggaggcatagattcttataaaaatcacaaaatttgttgtaaattagaggggagaaatttagaattaagtataaaaaggcagaattagtatagagtacattcattaaacatttttgtcaggattatttcccgtaaaaacgtagtgagcacttttcatatactaatttagttgtacatttaactttgtataatacagaaatctaaatatatttaatgaattcaagcaatatatcacttgaccaagaaattggaatttcaaaatgttcgtgcgggtatataccagatgagtacagtgagtagttttatgtatcaccagactgggttattgccaagttatatatcaccaaaagctgtatgactggatgttctggttacctggtttacaaaattatcagagtagtaaaactttgatatatatgaggatattaaaactacactaagtatcatttgattcgattcagaaagtactttgatatctctcagtgcttcagtgctatcattgtgagcaattgtcttttatatacggtactgtagccatactaggcctgtctgtggcattctctagatgtttcttttttacacaataaattccttatatcagcttgaaaaaaaaaaaaaaaaaa

SEQ ID NO: 195 - DNA, 2208 bp, Homo sapiens
aacgcacttggcgcgcggcgcgggctgcagacggctgcgaggcgctgggcacaggtgtcctgatggcaaatttcaagggccacgcgcttccagggagtttcttcctgatcattgggctgtgttggtcagtgaagtacccgctgaagtactttagccacacgcggaagaacagcccactacattactatcagcgtctcgagatcgtcgaagccgcaattaggactttgttttccgtcactgggatcctggcagagcagtttgttccggatgggccccacctgcacctctaccatgagaaccactggataaagttaatgaattggcagcacagcaccatgtacctattctttgcagtctcaggaattgttgacatgctcacctatctggtcagccacgttcccttgggggtggacagactggttatggctgtggcagtattcatggaaggtttcctcttctactaccacgtccacaaccggcctccgctggaccagcacatccactcactcctgctgtatgctctgttcggagggtgtgttagtatctccctagaggtgatcttccgggaccacattgtgctggaacttttccgaaccagtctcatcattcttcagggaacctggttctggcagattgggtttgtgctgttcccaccttttggaacacccgaatgggaccagaaggatgatgccaacctcatgttcatcaccatgtgcttctgctggcactacctggctgccctcagcattgtggccgtcaactattctcttgtttactgccttttgactcggatgaagagacacggaaggggagaaatcattggaattcagaagctgaattcagatgacacttaccagaccgccctcttgagtggctcagatgaggaatgagccgagatgcggagggcgcagatgtcccactgcacagctggaatgaatggagttcatcccctccacctgaatgcctgctgtggtctgatcttaagggtctatatatttgcacctcctcattcaacacagggctggaggttctacaacaggaaatcaggcctacagcatcctgtgtatcttgcagttgggatttttaaacatactataaagtctgtgttggtatagtacccttcataaggaaaaatgaagtaatgcctataagtagcaggcctttgtgcctcagtgtcaagagaaatcaagagatgctaaaagctttacaatggaagtggcctcatggatgaatccggggtatgagcccaggagaacgtgctgcttttggtaacttatccctttttctcttaagaaagcaggtactttcttattagaaatatgttagaatgtgtaagcaaacgacagtgcctttagaattacaattctaacttacatattttttgaaagtaaaataattcacaagctttggtattttaaaattattgttaaacatatcataactaatcataccagggtactgcaataccactgtttataagtgacaaaattaggccaaaggtgatttttttttaaatcaggaagctggttactggctctactgagagttggagccctgatgttctgattcttcaaagtcaccctaaaagaagatctgacaggaaagctgtataatgagatagaaaaacgtcaggtatggaaggctttcagttttaatatggctgaaagcaaaggataacgaattcagaattagtaatgtaaaatcttgataccctaatcttgcttctggatctgttctttttttaaaaaaacttccttcaccgcgcctataatcctagcactttgggaggccgaggcaggcagatcacggggtcaggagatcaagaccatcctggctaacatggtgaaaccccgtctctactgaaaatacaaaaaattagccgggtgtggtggcgggcgcctgtagttccagctactcgggaggctgaggcaagagaatggcatgaacccggtaggggagcttgcagtgagcccagatcatgccactgtactccagcctaggtgacagagcaagactctgtctcaaaaacaagcaaacagacttccttcaacaaatatttattaaatatccactttgcaacagcactgaaatggctgtaaggactcctgagatatgtgtccagcaaggagtttacagtcaaacaggagagacatgcctgtagttacatccagtgtgatgggtgctgagaggcaagtacaaaccacgatg

SEQ ID NO: 196 - DNA, 1678 bp, Homo sapiens
  misc_feature (523)..(523): n is a, c, g, or t
tcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgcccgcccgggcaccggtaccctgccggtattcggcatgcaggcgcgctacagcctgagagatgaattccctctgctgacaaccaaacgtgtgttctggaacggtgcttcggaggagctgctgtggcttatcaagggatccacaaacgctatagacctgtcttccccggcagcgaaaatctcgggatgccactggatcccgacactctctggacaccctgggattctccaccagagaagaacgcgacttgggcccagtttgtggctctcagcggaggcctcctgtggcagaatacatacatttccaatcagatcacttcccggacacggaccntgaccagcctgccaaaaagtggatttccccccaccccagaacccancccctgacgcacagaaaccaacccattcgttgttgccgccttgcgaaccccaaccagaatctctcccccctggccggcgcgcctgccgctgccaatgcccctatggcggcctcttggcccgcaccttccaattggtcgccctgcgcaaccagcgagaaaacactggcccgcccgtctcccccccgctccgcctaccccacttaatgcgcctccgtggcatgacgcacgcgtttggtgtccgccgccgtctcatgtccgcgcggtgtggacccccttttctctcgcggcacatcccccctattcccttgccctttggggggcaccccctctagacccgcgcttctcttctcgtccggtgggggacattggtttgcctgccgcggcgggggcgntaaaaataaaaacagcctgttagcccggcccagtacccccccccggccggggccgccttncgtttgcatttataccccaacccataaagccgcgcccctttagcnccntaacttttgtggtgtggcctcccccctttttcccggggagcagcaacggacatctgtacactaatgctggccccgacctttcccaaaaaccccccgcccgtgtcccgtataaatttggtgccaancctgacgngttctcccccgccctcgccccgttggccgcccgtttaaagcccccccggtggttgcgccgcccaacgagtccacctatagttaantccaccaacacccccaccttttcctccccgccgcatcttccccacgtacccccttttgtcgcgagatggccactcccccccccctgtttgtttaaaacaacgagaatggtgctgccaacgctggtcttttccccccccggaccgcgaccgccagggggaatacgtaccataagcccccgcgcccnccttttttcccccctccccgccaatcaagatccgccgtccattagacgtattatttttcccgcgatacacgaaaaaacagggccgcccatttataactaaattcccgtcgccgccgcgcggatatgtttcccaaaataccacccccccccccccattttctttgcccccaactcctgcgcaccggtgttcaccagcctcgcgccgc

SEQ ID NO: 197 - DNA, 1611 bp, Homo sapiens
ggacgcgtgggtcgacccacgcgtccggacccacgcgtccggtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatggtgagtgattaagtgcccagaaccccagccttccatccaattttcagtagcctccttttttccgtcagcttttttgctagacataggggtaatgtaatttgctccctcctgggaaagaagttcatacaccccacctacaccatttcttccagcagtccctcctcccaattccatccccccacacgaagttatctcgaacacttccctgaagtcatacaagaccctccctatccagtgtgtccctacttcctagccccaaccaagctttacccacacccaactccccgcccttcttggtatttctagcctatgaatttggttgctttattttggatcagagtgatgagattaaggggaggctgggcgcggtagctcacaccttataatcccaaagtgctgggattacaggcgtgagccaccgcgcccggccagcaactaatattctaattgaactaaagcacaggatgccaatttacaatccttagaccaaagagtcactgatgtctccaccagataagaggaaagcatcaggctaggcatagtggctcacacctgtaatctcagcactttgggaggctgaggcaggcagatcacatgagcccaggagtttgagactggcctgggcaacatggtgaaaccctgtctctaaaataaaaactaaactaaaaaaactttttaaaaaggcagtggggagcatcagaaccagctcaacagtttgtctactgtccggtcccagagaaactcaagattctagcaagccccttgtgtggggcttgggttgggacatgaggctgctgctggagcttactctgcaactgtttctccaaatgccaggtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttaaaaaaaaaataaaaaaaaaaaaaaaaaaaaaaaaaaaaaaaaaaaaaaaaaaaaa

SEQ ID NO: 198 - DNA, 4926 bp, Homo sapiens
cgcagtctctctctctctccctcctccgggaggaactgccgcgctccggctgactcctccgccggcgggcggggcgggggagggggcttcgggcgcgctgggaaccgcgggacccggacctgggcgccgcccgccgggggacgcgcggcccccgcttccgccgggccccgctgagctctagacaaacctccgcttcagaaataggctgcgggcggccggctaggaggcttggcccccaccccgggacccccgccgtccccgggccggccggccggtgggcacgatgagccaggtgctggggaagccgcagccgcaggacgaggacgacgcggaggaggaggaggaggaggatgagctggtggggctagcggactacggagacgggcccgactcctccgacgccgatccggacagcggcacagaggagggagttctggacttcagtgaccccttcagcactgaagtgaagccgagaatcctgctcatgggcctgaggagaagcggcaagtcgtctattcagaaagttgtctttcacaaaatgtctcccaacgaaactctgttcttggagagcactaataagatatgccgggaagatgtttccaacagctcctttgtcaattttcagatttgggacttcccaggacagattgacttttttgaccctacatttgactatgagatgatcttccggggaacaggagcactgatatttgtcattgactcacaggatgattacatggaagccctggccaggctccacctcacggtgaccagggcctacaaagtgaatactgacatcaacttcgaggtgtttattcataaagtggatggtctgtcagatgaccacaaaattgaaacccaaagagatattcaccagagggcaaacgatgaccttgcagatgctggattagaaaaaattcacctcagcttttatctgacaagcatatatgatcattcaatatttgaagcttttagcaaagttgttcagaaactgattccacaactcccaactctggagaatttgctgaacatctttatctcaaattctggaattgaaaaggcatttctatttgatgtggtcagtaaaatttatattgcaactgatagtactccggtggatatgcaaacctatgagctctgctgtgatatgatagatgtggttattgacatctcttgtatttatggtctcaaagaagatggagcaggaaccccctatgacaaggaatccacagccatcataaagcttaataatacaaccgtgctttatttaaaagaggtgacaaagttcctggctctcgtttgctttgtcagagaggaaagctttgaaagaaaagggctaattgactataattttcattgcttccggaaggccattcatgaagtttttgaggtgagaatgaaagtagtaaaatctcgaaaggttcagaatcggctgcagaagaaaaagagagccacccctaatgggacccctagagtgctgctgtaggtgaggtttcaggaatgtcttttgaaatcagaccttatccatgaggctgctgcgccatgttgcactaaaggaagaggaagaaggagattgggacacataccattgatttgttgttaaaaaaaaaaaattcctgcaaccctcttgatcttctcttttataaataaagtaagcactttgaagcaaaaacttgtatattaacagtgatgtgaaatccattgtcatttcattacacaaatgtaaacttttatggtctgtagtcaaaaaaatcccgtgtgagaactgccaggaattgtacatattttgcactttttcatgtttctcattgaactgaactttgataaaacgacttttctaagctttttttctgtacttggtgtcaaggacatgcatactgtagtccatatctatatggcaatcagaaattaatcaaaaagtgatgcattggtaatgactttttgtaaatttggaaatctttgctaccaattgttgagaaaaatcatttttcagtggagctggaacagattggagctacaagctccaggagcaataagaactgtcccctatttataatgggtgtaaacagttttgtagaataatgctagcaccagacttacctaaaaatttctatagcagtggctgtgcttccctgctcaacggtttttatgaagctgtttacctcaacacacatctctataatcactttatacagagaggttatttctttttgttgcattagtattcttttgaaactttgggaccagatttccaaaatggtgccgaacactggagagaagtaagaatgtcactgaattgtagggtttctggaggcttttctgtacctaccacccagggctaaagtaacatcagaggcctaaagttgttccaaaagtatgtgattggcaactgcagactaaaaaacatagatacaattctggacttttggccctgtgcgatggtctggtgtgctgcatttaaaatgcttattcaggaccagttctttattgctccatgaccatagtgaatagaacaaatcgcagaaccccaccatggagctcaatcctgttagtcacttttgtcacctccacatctccttctcactggtgataacatgcctcatgtactccacttgttcccaccttatgattaagccaagctcagcctgccaccagcatgctctgcaaggctgaagagtcatcctgaagacccctaaaggtcagtgggaaaaggatggctggagagacattacaattagctgtgtaattgtttctgtgaaattatttcacttatgtttactttagactaacaggaaattaagagtcctaaatctacccctatgccaaatcattccaagtagataattttacgtgcatctcaagggttagcaccctaaggcatgcttgtggggcattagaaaatgagatttttttttttttaaagcagagcctcctaagaacatcaaagttggtcctagcaaaatatataaagtccctaaagcaacttatacttgaaactttttttttttttttttttagaggggggcctcattctgttgcttgtgctggaatacattggtacaatcatagctcactgttacctcttgggctcaagggatccttccacctcagcctccctagtagctagaactacaggtgtgcacaaccacgcccggctaattcttaaattttgtttttgtagagacaggatctcactgtgttgcccaagatggtctcaaactcctggccccaagcaatcctcctgccttggcctcctcaaatgctgagattacaggcctgagctactgtgcccagcctaaactttcccacttctctctgtggcttctttccaacctctctccttcctctccccaagtcctgtttctttgaagctggtaactgaatttaagatgatatctggttggtgtttaaggtttgagcctcccaaggttctgtgcattttgaaaggagatttctaaaaataattaaggtgccctaactcctttcctcatgattcctactccgaaacctggatggttaggagcccagggctccctgatttccagagctatatcctgttggacctttgccaacagacctgacacttaggggttattgttataaatctaattctctaatattttttacatgttgtttcactttgaataagcaaatgaagaatcagttttctaatatgactttatcctcaagctagagacactagcctatttggtaaatcacacattacttaggtatatttattactataaccaggttggagcttccatgtttaagctgggtatatgatgggtttttgttaaaatgtgccttaaaaagcctattacttcaagagcaaatgattctttgggggaaaggcaaaaataattctatgacatagggcccaagttcatggtagtaagtgtactctttgattaatcacacgctaatatagattactgcctctaactttgtaagtgtggcaatgacttcttaattaaagaaagatgcaggagttatgtctaagcgttcagtttttcaaatctgtgttattggaaatgtcttcaagtcattttgcattgtatttttgatatgagaggcagcttattgcgatgtgtatggccatgtttcattctcaaatttaattctataaatacaaatcctaaatacatggctacagcaactgcactggaacatttttgcttggttttagggattgagaacttgccttgcaggtttccttcctcaaaaggagcagggcagtcctttccctgttgagtcaattagaatttttacatagaggtgagactgtgaattattttggttattttcagtgatgtagattagtgtgaatgaccagggtggaatgtttttgaaggaatataaagcaaaaactggttgacattcacaaactgttcttttgtgaacatattttggacccttaaatatgactaaaatcacagcaatattgttacatacgggttatatgccaactctgtttgaaatatactctggaaaaacagctgaattgtcttggttattaaagtatggtatgtattcaacttgtacagactggatgtaatttgtaatcaggtatagtccatgttttactttaagcagtacatcacttaataaccattgttaagccattgctttcaagaatgttaactgccaatttaaaagcatgtgtcctaggttcatgctttggtaaagctctcatttcaagtgtattcatagctaagctttctgggagcagaattgtctctttggtgaaaaggaagtacagcctttcctgtttctgaggttgcttaccatacatgtatgtcactgtttcattggccctgttacatccatttggtaaaatttatttgtcctgattaaccagctctcattttatggaaatgatgataaatctcactacttaaatttaatttatgcttttatttttaa

SEQ ID NO: 199 - DNA, 751 bp, Homo sapiens
cgccgcccagcgcgggccgccgttataaagcagccgccggcgccgggtgcctcacagcacgctgccacgccgacgcagacccctctctgcacgccagcccgcccgcacccaccatggccacagttcagcagctggaaggaagatggcgcctggtggacagcaaaggctttgatgaatacatgaaggagctaggagtgggaatagctttgcgaaaaatgggcgcaatggccaagccagattgtatcatcacttgtgatggtaaaaacctcaccataaaaactgagagcactttgaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgaccaagctcagttcaatgagcaaatctccatactgtttctttcttttttttttcattactgtgttcaattatctttatcataaacattttacatgcagctatttcaaagtgtgttggattaattaggatcatccctttggttaataaataaatgtgtttgtgctaataaaaaaaaaaaaaaaaaaaaa

SEQ ID NO: 200 - DNA, 1141 bp, Homo sapiens
agtgcgtctggccggcgctttatagctgcagcctgggcggctccgctagctgtttttcgtcttccctaggctatttctgccgggcgctccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtctgcagagaattcaccgagcgtgagcaaggagaagtccgcttctctgccgtggctctctgcaaggcagcctaatgctctgtgggagggactttgctgatttcccctcttcccttcaacatgaaaatatatacccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatctaacgctttaaatggctactttggtttctgtctgtaagttaagaccttggatgtggtttaattgtttgtcctcaaaaggaataaaacttttctgctgataagataaaaaaaaaaaaaaaaaa

SEQ ID NO: 201 - DNA, 778 bp, Homo sapiens
atatagcagcggcggcggtggcggcggccacaccgggcggcggacacgtggagggacccggcccgcgccttctgcccctgctgccggccgcgccatgcggtgagcgccccaggccgccagagcccacccgacccggcccgacgcccggacctgccgcccagacccgccaccgcacccggaccccgacgctccgaacccgggcgcagccgcagctcaagatggcccgaggcagcgccctcctgctcgcctccctcctcctcgccgcggccctttctgcctctgcggggctctggtcgccggccaaggaaaaacgaggctggaccctgaacagcgcgggctacctgctgggcccacatgccgttggcaaccacaggtcattcagcgacaagaatggcctcaccagcaagcgggagctgcggcccgaagatgacatgaaaccaggaagctttgacaggtccatacctgaaaacaatatcatgcgcacaatcattgagtttctgtctttcttgcatctcaaagaggccggtgccctcgaccgcctcctggatctccccgccgcagcctcctcagaagacatcgagcggtcctgagagcctcctgggcatgtttgtctgtgtgctgtaacctgaagtcaaaccttaagataatggataatcttcggccaatttatgcagagtcagccattcctgttctctttgccttgatgttgtgttgttatcatttaagattttttttttttggtaattattttgagtggcaaaataaagaatagcaatta

SEQ ID NO: 202 - DNA, 3115 bp, Homo sapiens
ccaccatatcggtcccgtatttcacattgataaggtcctgtttcatttctcgtgacattgggtagaatgaggatcctgttttcaatgggtcgctttaccctgggactgacagggaggctctgaccatttagccaccaaatgtaggtgtagttctcactcttaggttcaccccgcggccgatcgtcccccatacctcggccatgcggcccctgctgctactggccctgctgggctggctgctgctggccgaagcgaagggcgacgccaagccggaggacaaccttttagtcctcacggtggccactaaggagaccgagggattccgtcgcttcaagcgctcagctcagttcttcaactacaagatccaggcgcttggcctaggggaggactggaatgtggagaaggggacgtcggcaggtggagggcagaaggtccggctgctgaagaaagctctggagaagcacgcagacaaggaggatctggtcattctcttcacagacagctatgacgtgctgtttgcatcggggccccgggagctcctgaagaagttccggcaggccaggagccaggtggtcttctctgctgaggagctcatctacccagaccgcaggctggagaccaagtatccggtggtgtccgatggcaagaggttcctgggctctggaggcttcatcggttatgcccccaacctcagcaaactggtggccgagtgggagggccaggacagcgacagcgatcagctgttttacaccaagatcttcttggacccggagaagagggagcagatcaatatcaccctggaccaccgctgccgtatcttccagaacctggatggagccttggatgaggtcgtgctcaagtttgaaatgggccatgtgagagcgaggaacctggcctatgacaccctcccggtcctgatccatggcaacgggccaaccaagctgcagttgaactacctgggcaactacatcccgcgcttctggaccttcgaaacaggctgcaccgtgtgtgacgaaggcttgcgcagcctcaagggcattggggatgaagctctgcccacggtcctggtcggcgtgttcatcgaacagcccacgccgtttgtgtccctgttcttccagcggctcctgcggctccactacccccagaaacacatgcgacttttcatccacaaccacgagcagcaccacaaggctcaggtggaagagttcctggcacagcatggcagcgagtaccagtctgtgaagctggtgggccctgaggtgcggatggcgaatgcagatgccaggaacatgggcgcagacctgtgccggcaggaccgcagctgcacctactacttcagcgtggatgctgacgtggccctgaccgagcccaacagcctgcggctgctgatccaacagaacaagaatgtcattgccccgctgatgacccggcatgggaggctgtggtcgaacttctggggggctctcagtgcagatggctactatgcccgttccgaggactacgtggacattgtgcaggggcggcgtgttggtgtctggaatgtgccctatatttcaaacatctacttgatcaagggcagtgccctgcggggtgagctgcagtcctcagatctcttccaccacagcaagctggaccccgacatggccttctgtgccaacatccggcagcaggatgtgttcatgttcctgaccaaccggcacacccttggccatctgctctccctagacagctaccgcaccacccacctgcacaacgacctctgggaggtgttcagcaaccccgaggactggaaggagaagtacatccaccagaactacaccaaagccctggcagggaagctggtggagacgccctgcccggatgtctattggttccccatcttcacggaggtggcctgtgatgagctggtggaggagatggagcactttggccagtggtctctgggcaacaacaaggacaaccgcatccagggtggctacgagaacgtgccgactattgacatccacatgaaccagatcggctttgagcgggagtggcacaaattcctgctggagtacattgcgcccatgacggagaagctctaccccggctactacaccagggcccagtttgacctggcctttgtcgtccgctacaagcctgatgagcagccctcactgatgccacaccatgatgcctccaccttcaccatcaacatcgccctgaaccgagtcggggtggattacgagggcgggggctgtcggttcctgcgctacaactgttccatccgagccccaaggaagggctggaccctcatgcaccctggacgactcacgcattaccatgaggggctccccaccaccaggggcacccgctacatcgcagtctccttcgtcgatccctaattggccaggcctgaccctcttggacctttcttctttgccgacaaccactgcccagcagcctctgggacctcggggtcccagggaacccagtccagcctcctggctgttgacttcccattgctcttggagccaccaatcaaagagattcaaagagattcctgcaggccagaggccggaacacacctttatggctggggctctccgtggtgttctggacccagcccctggagacaccattcacttttactgctttgtagtgactcgtgctctccaacctgtcttcctgaaaaaccaaggcccccttcccccacctcttccatggggtgagacttgagcagaacaggggcttccccaagttgcccagaaagactgtctgggtgagaagccatggccagagcttctcccaggcacaggtgttgcaccagggacttctgcttcaagttttggggtaaagacacctggatcagactccaagggctgccctgagtctgggacttctgcctccatggctggtcatgagagcaaaccgtagtcccctggagacagccactccagagaacctcttgggagacagaagaggcatctgtgcacagctcgatcttctacttgcctgtggggaggggagtgacaggtccacacaccacactgggtcaccctgtcctggatgcctctgaagagagggacagaccgtcagaaactggagagtttctattaaaggtcatttaaaccac

SEQ ID NO: 203 - DNA, 1752 bp, Homo sapiens
agggcgcagcaggccaagggggaggtgcgagcgtggacctgggacgggtctgggcggctctcggtggttggcacgggttcgcacacccattcaagcggcaggacgcacttgtcttagcagttctcgctgaccgcgctagctgcggcttctacgctccggcactctgagttcatcagcaaacgccctggcgtctgtcctcaccatgcctagcctttgggaccgcttctcgtcgtcgtccacctcctcttcgccctcgtccttgccccgaactcccaccccagatcggccgccgcgctcagcctgggggtcggcgacccgggaggaggggtttgaccgctccacgagcctggagagctcggactgcgagtccctggacagcagcaacagtggcttcgggccggaggaagacacggcttacctggatggggtgtcgttgcccgacttcgagctgctcagtgaccctgaggatgaacacttgtgtgccaacctgatgcagctgctgcaggagagcctggcccaggcgcggctgggctctcgacgccctgcgcgcctgctgatgcctagccagttggtaagccaggtgggcaaagaactactgcgcctggcctacagcgagccgtgcggcctgcggggggcgctgctggacgtctgcgtggagcagggcaagagctgccacagcgtgggccagctggcactcgaccccagcctggtgcccaccttccagctgaccctcgtgctgcgcctggactcacgactctggcccaagatccaggggctgtttagctccgccaactctcccttcctccctggcttcagccagtccctgacgctgagcactggcttccgagtcatcaagaagaagctgtacagctcggaacagctgctcattgaggagtgttgaacttcaacctgagggggccgacagtgccctccaagacagagacgactgaacttttggggtggagactagaggcaggagctgagggactgattcctgtggttggaaaactgaggcagccacctaaggtggaggtgggggaatagtgtttcccaggaagctcattgagttgtgtgcgggtggctgtgcattggggacacatacccctcagtactgtagcatgaaacaaaggcttaggggccaacaaggcttccagctggatgtgtgtgtagcatgtaccttattatttttgttactgacagttaacagtggtgtgacatccagagagcagctgggctgctcccgccccagcccggcccagggtgaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaactgtttacatgaagataagatactcactgttcatgaatacacttgatgttcaagtattaagacctatgcaatattttttacttttctaataaacatgtttgttaaaacagtt

SEQ ID NO: 204 - DNA, 640 bp, Homo sapiens
tcgggcctccgaaaccatgaactttctgctgtcttgggtgcattggagccttgccttgctgctctacctccaccatgccaagtggtcccaggctgcacccatggcagaaggaggggggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatct

SEQ ID NO: 205 - DNA, 1449 bp, Homo sapiens
ctggatagaacagctcaagccttgccacttcgggcttctcactgcagctgggcttggacttcggagttttgccattgccagtgggacgtctgagactttctccttcaagtacttggcagatcactctcttagcagggtctgcgcttcgcagccgggatgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttaggatttaggcgcccatggtacaaggaatagtcgcgcaagcatcccgctggtgcctcccgggacgaaggacttcccgagcggtgtggggaccgggctctgacagccctgcggagaccctgagtccgggaggcaccgtccggcggcgagctctggctttgcaagggcccctccttctgggggcttcgcttccttagccttgctcaggtgcaagtgccccagggggcggggtgcagaagaatccgagtgtttgccaggcttaaggagaggagaaactgagaaatgaatgctgagacccccggagcaggggtctgagccacagccgtgctcgcccacaaactgatttctcacggcgtgtcaccccaccagggcgcaagcctcactattacttgaactttccaaaacctaaagaggaaaagtgcaatgcgtgttgtacatacagaggtaactatcaatatttaagtttgttgctgtcaagattttttttgtaacttcaaatatagagatatttttgtacgttatatattgtattaagggcattttaaaagcaattatattgtcctcccctattttaagacgtgaatgtctcagcgaggtgtaaagttgttcgccgcgtggaatgtgagtgtgtttgtgtgcatgaaagagaaagactgattacctcctgtgtggaagaaggaaacaccgagtctctgtataatctatttacataaaatgggtgatatgcgaacagcaaacc

SEQ ID NO: 206 - DNA, 1859 bp, Homo sapiens
gactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccactgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaaaaaaaaaaaaaaaaaaa

SEQ ID NO: 207 - DNA, 1185 bp, Homo sapiens
atgtctcgggagatgcaggatgtagacctcgctgaggtgaagcctttggtggagaaaggggagaccatcaccggcctcctgcaagagtttgatgtccaggagcaggacatcgagactttacatggctctgttcacgtcacgctgtgtgggactcccaagggaaaccggcctgtcatcctcacctaccatgacatcggcatgaaccacaaaacctgctacaaccccctcttcaactacgaggacatgcaggagatcacccagcactttgccgtctgccacgtggacgcccctggccagcaggacggcgcagcctccttccccgcagggtacatgtacccctccatggatcagctggctgaaatgcttcctggagtccttcaacagtttgggctgaaaagcattattggcatgggaacaggagcaggcgcctacatcctaactcgatttgctctaaacaaccctgagatggtggagggccttgtccttatcaacgtgaacccttgtgcggaaggctggatggactgggccgcctccaagatctcaggatggacccaagctctgccggacatggtggtgtcccacctttttgggaaggaagaaatgcagagtaacgtggaagtggtccacacctaccgccagcacattgtgaatgacatgaaccccggcaacctgcacctgttcatcaatgcctacaacagccggcgcgacctggagattgagcgaccaatgccgggaacccacacagtcaccctgcagtgccctgctctgttggtggttggggacagctcgcctgcagtggatgccgtggtggagtgcaactcaaaattggacccaacaaagaccactctcctcaagatggcggactgtggcggcctcccgcagatctcccagccggccaagctcgctgaggccttcaagtacttcgtgcagggcatgggatacatgccctcggctagcatgacccgcctgatgcggtcccgcacagcctctggttccagcgtcacttctctggatggcacccgcagccgctcccacaccagcgagggcacccgaagccgctcccacaccagcgagggcacccgcagccgctcgcacaccagcgagggggcccacctggacatcacccccaactcgggtgctgctgggaacagcgccgggcccaagtccatggaggtctcctgttaa

SEQ ID NO: 208 - DNA, 2438 bp, Homo sapiens
ataagccagagcctagaccagtgagccaactgtgcgaaccagacccggcagccttgctcagttcagcatagcggagcggatccgatcggatcggagcggatcggagcacaccggagcaggctcatcgagaaggcgtctgcgagaccatggagaacggatacacctatgaagattataagaacactgcagaatggcttctgtctcacactaagcaccgacctcaagttgcaataatctgtggttctggattaggaggtctgactgataaattaactcaggcccagatctttgactacggtgaaatccccaactttccccgaagtacagtgccaggtcatgctggccgactggtgtttgggttcctgaatggcagggcctgtgtgatgatgcagggcaggttccacatgtatgaagggtacccactctggaaggtgacattcccagtgagggttttccaccttctgggtgtggacaccctggtagtcaccaatgcagcaggagggctgaaccccaagtttgaggttggagatatcatgctgatccgtgaccatatcaacctacctggtttcagtggtcagaaccctctcagagggcccaatgatgaaaggtttggagatcgtttccctgccatgtctgatgcctacgaccggactatgaggcagagggctctcagtacctggaaacaaatgggggagcaacgtgagctacaggaaggcacctatgtgatggtggcaggccccagctttgagactgtggcagaatgtcgtgtgctgcagaagctgggagcagacgctgttggcatgagtacagtaccagaagttatcgttgcacggcactgtggacttcgagtctttggcttctcactcatcactaacaaggtcatcatggattatgaaagcctggagaaggccaaccatgaagaagtcttagcagctggcaaacaagctgcacagaaattggaacagtttgtctccattcttatggccagcattccactccctgacaaagccagttgacctgccttggagtcgtctggcatctcccacacaagacccaagtagctgctaccttctttggccccttgctggagtcatgtgcctctgtccttaggttgtagcagaaaggaaaagattcctgtccttcacctttcccactttcttctaccagacccttctggtgccagatcctcttctcaaagctgggattacaggtgtgagcatagtgagaccttggcgctacaaaataaagctgttctcattcctgttctttcttacacaagagctggagcccgtgccctaccacacatctgtggagatgcccaggatttgactcgggccttagaactttgcatagcagctgctactagctctttgagataatacattccgaggggctcagttctgccttatctaaatcaccagagaccaaacaaggactaatccaatacctcttggattttatttaatgtcataatgttgtcagaataaagagaaagatgaaataatttcatttttttgtgtaacttggtatggggctggggcacagaccaagattgacatgaaaggatgtgagatcgcatgtcttgtgtgactatctgcttctcagacaagcagttaggaactgagatgagatagtatgtgagggcagcaaaggatgaagaagggcaaaatgatgaaaggtgaggtggaaagaggttatgagatggtaaagaaaagttaacttctggcacttgattgccacttctgtcaggctggtcctgcctctctcccttgccttctgattgtttcatttcctgtttatttgatcatatctgaattagttcactggttagcctcttccttagttcccacttccttaccaaagccctaattatatttcctcttgtttgccttttctctcctactcttctctaacatctgcagccacactctccattcactccatgctgacaaggcagtggcaaacacttttctctgctgccagccactccactgttgactggattgctgccagccccaggcaaacctgtgaagttgtttcatactctgcttctctttgagtgcctccttctctccttcctcttcctttctgggctccagtctttctcttcacttgtgcttgtcagaacctccctgtgatactgcctccaggcatttcccccatgttggctcaccgcactattatctttgcttatcaacttgcattcagctggctggcatgtttcaaaaccacactgccctcccaggcctgtgtgccttttgagaaagaccagtgctggatgagcctctagtaatgacaacattttagttgttagtggtataatacggaagagatattttgcacaggctgctttggagaactttcaaattatcctttgtttggtaactgacctacttaactgcccaatacaaagaaaaagcaaaaaaaaaaaaaaaaaa

SEQ ID NO: 209 - DNA, 1588 bp, Homo sapiens
gaattcgcccttcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgag

SEQ ID NO: 210 - DNA, 3669 bp, Homo sapiens
gcgaggccccaagctggcccgggagaggactctgcgggcgaagtggctgcgcaaggagagaacttttcctgcacaaggaacgcctcgtggggagacccaaggcaggagcggtccggagccggctgcggcgtgtgcggccggccttgggacagcgatcgccgcgggtggcaacagagagccccaagcaaaagtgggagcaggagcttggaggtgagcacaggaagccccacttgaggcttttacgcagcctctagtctctgtttcttctggaataggcaagtgtcctttcaactctaagagaccagcagaggccactgtcccttaagactgccggagtcctcaccacttctccaggattccagaggagactgtggcgatggtgaatgaaggtcccaaccaggaagagagcgatgacacccctgtgccggagtccgcactccaagcggaccccagcgtctcggtccatcccagcgtctcggtccatcccagcgtctccatcaaccccagcgtctctgtccaccccagcagttcggcccaccccagtgccttagcccaacccagtggcttggctcaccccagtagctcgggccctgaggacctcagcgtgatcaaggtgagcaggcgccgttgggccgtggtcctggtgtttagctgctactccatgtgcaactcctttcagtggatccagtacggctccatcaataacatcttcatgcacttctacggtgtcagtgcctttgccattgactggctgtccatgtgctacatgctgacttacatccctctgctcctgccagtggcttggctgctggagaagttcggcctgcgcaccattgctctcactggctcggctctcaactgcctgggggcctgggtgaagctgggcagcctgaagccgcatctctttccggtcaccgtggtgggccagctcatctgctctgtggcccaggttttcatcctgggcatgccctcccgcatcgcttccgtctggttcggggctaatgaggtttcaacagcctgctccgtggctgtctttggcaatcagcttggaattgcgattgggttcttggtccctcctgttttggtacccaacattgaagaccgggacgagcttgcctaccacatcagcatcatgttctatataataggaggtgtggccactctcctcctcatccttgtcatcattgtgttcaaggagaaacctaaatatccccccagcagggcccaatccctgagctatgccttgacctctcctgatgcctcatacttaggttccatcgcccggctcttcaaaaatctcaactttgtgctgcttgtcatcacctatggtctgaatgctggtgctttttatgccttgtccactcttctgaatcgcatggtgatctggcactacccgggggaagaagtgaatgctggaagaattggcctgacgatcgtcattgcaggaatgcttggggctgtgatctcaggaatctggctggataggtccaaaacctacaaagagacaaccctggtagtctatatcatgacactggtgggcatggtggtgtacacgtttaccttgaacctgggacacctgtgggtagtgttcatcactgctggcacaatgggcttctttatgactggctatctcccactgggatttgagtttgctgtggagctcacgtacccagaatcagaaggcatctcctccggcctcctcaacatatctgcacaggtatttgggatcatctttaccatctcccagggccagattattgacaactatggaaccaagcctgggaacatcttcctgtgtgtgttccttactcttggagcagccctcactgcattcattaaggcagatctccggagacagaaagcaaacaaagaaactcttgagaacaaactccaagaggaggaggaggagagcaacaccagcaaagtgcccactgctgtgtcagaggatcatctctgagaggaaggtggtgacaactcagggaacacgaacaccccaccttttccttcagcacagctctcaccgccagcacaaagggcttcgctagagatgtttttggagggaatcagtgggactatttgtggcatggatggcctattcctcctagaacccacgtaagagcttggatgatttagttggagaaaattgcacctatcaccaaatgcaaatttgattcccacctccacccccttttaggttatgggagttggtgttgggacagggtggcagagaatattggagtcaatcctagcttggtctcttgccttccctcttttcctccatccatcgtggacaatgcctgcaaaattttcacaggaagaaagcctattcaggatattaacttgaaatttccagtgtcctaagagcctctcatgaagcccagttctaataagtggcaagctgctctgccggggtcatctcctgggtcatcggactgattgctcaagttctgcaggagaggaagcaccattagaacaactccatcagaacagctccaccgggacttgtgggcctaaattttcctggcctaacgggtctgtctccaaaccctctttcctaagagctgagcaaaccaaccataataaacttgacaaaagactttgttgtggccatgacagagataccgactcaggagggctacctacctaggtgtgatcatgctgggggctaccttctgagtatatttgtgaaagcacatatttgggaactctggtagcttgagttgggaatgggaaggttcttttttacagaagtacttccccagggacttctgtgtgtcacagtcacctctgatgcctttatcttgatgttgcattgggaatctcagccatcagcccaagtgcttgttttattccaaggcagggtaatccccgtcaacttactctaacctttgctgaaaactaatcttgattcattctactctgaaaatccaaaggtgcttctgagagataagagggaaggggtagaaggaaaggtgccccttgaaatgggaattgagcctgttagaattaaaagcttatctcacctctgctggggacagtatttgcaccaccaacccctctcctcacctgctttgagcgataatctttatcagatattctaaacttaaagggattccctttaaaccaactcaagctgatctttcctatctagcctgctgtttggctgtactcatgggctttggtaatatctcctaaaaatgaggttttggtaatttttcctatgcattgggcaactgtgatcgtgaccactgtgctgtcttgctccagccactgccctggcctcagcatatcagggcagcctgtgctggctgcaatactgtggtgcttgggccactgcctgagaggagccaggtttgtgtgtgtctgcatgtgtgtgtgtgtgtgtttgtacagattcaagcaatggatgcaaggaacatgctgtatgtaatagaagaaagaagtccacgttttcggcagaagtagtgagtcagtgtggaagagaggtgagggtgtgctttactttttgataaagagaaagatgtttactcataaacccttcaaaaggtattaacaaaatgtttaccaaacctattgctttattttaaaaacataatttgtgttttctatttgtaagatctgacatttcgaggcaataaaaacttctcagaaagtaaaaaaaaaaaaaaa

SEQ ID NO: 211 - DNA, 3164 bp, Homo sapiens
acctgtataccttttgaaacgtcacagtctctaatcgtgaacgatttggggcggagggctgaacaatgtgttttctagtgtgtcgaggtgtttataggctatgtgtgcctccaaactgtaaagtagtccagtatactttccaatgtataagtttgtagaccttaaacttttcttctggctaacttaaaatcgttgaattcactagtttgcataaacatttaagaatttgaaaacacggttgaaaaacagtgttaccaagaaattttgtaataacatgttcaaatgaagacaaaaattttacagtttaagacttaaattcttcgtccacagcaagtgaattcatggtattttacttttttgggaaatactggaaatgaagacctgcaactgtaatttgaaataaggaaaactttaattttcagtataaaaattgctcaaatagaattgcctgattttaatgacaaaagtatatgggagtccacatttatgtaagaaatgaaactataaaatgtataaataatttgcaaatcagaattgctgtcgaaagttttactataatgaaagatattttcatactctcaaaaatatagaggaaaggggccaagattatagtaccagtcacaatcttttgatgaggacgaaatgaatcaggtaacagactgggttgacccatcatttgatgattttctagagtgtagtggcgtctctactattactgccacatcattaggtgtgaataactcaagtcatagaagaaaaaatgggccttctacattagaaagcagcagatttccagcgagaaaaagaggaaatctatcttccttagaacagatttatggtttagaaaattcaaaagaatatctgtctgaaaatgaaccatgggtggataaatataaaccagaaactcagcatgaacttgctgtgcataaaaagaaaattgaagaagtcgaaacctggttaaaagctcaagttttagaaaggcaaccaaaacagggtggatctattttattaataacaggtcctcctggatgtggaaagacaacgaccttaaaaatactatcaaaggagcatggtattcaagtacaagagtggattaatccagttttaccagacttccaaaaagatgatttcaaggggatgtttaatactgaatcaagcttccatatgtttccctatcagtctcagatagcagttttcaaagagtttctactaagagcgacaaagtataacaagttacaaatgcttggagatgatctgagaactgataagaagataattctggttgaagatttacctaaccagttttatcgggattctcatactttacatgaagttctaaggaagtatgtgaggattggtcgatgtcctcttatatttataatctcggacagtctcagtggagataataatcaaaggttattgtttcccaaagaaattcaggaagagtgttctatctcaaatattagtttcaaccctgtggcaccaacaattatgatgaaatttcttaatcgaatagtgactatagaagctaacaagaatggaggaaaaattactgtccctgacaaaacttctctagagttgctctgtcagggatgttctggtgatatcagaagtgcaataaacagcctccagttttcttcttcaaaaggagaaaacaacttacggccaaggaaaaaaggaatgtctttaaaatcagatgctgtgctgtcaaaatcaaaacgaagaaaaaaacctgatagggtttttgaaaatcaagaggtccaagctattggtggcaaagatgtttctctgtttctcttcagagctttggggaaaattctatattgtaaaagagcatctttaacagaattagactcacctcggttgccctctcatttatcagaatatgaacgggatacattacttgttgaacctgaggaggtagtagaaatgtcacacatgcctggagacttatttaatttatatcttcaccaaaactacatagatttcttcatggaaattgatgatattgtgagagccagtgaatttctgagttttgcagatatcctcagtggtgactggaatacacgctctttactcagggaatatagcacatctatagctacgagaggtgtgatgcattccaacaaagcccgaggatatgctcattgccaaggaggaggatcaagttttcgacccttgcacaaacctcagtggtttctaataaataaaaagtatcgggaaaattgcctggcagcaaaagcactttttcctgacttctgcctaccagctttatgcctccaaactcagctattgccataccttgctctactaaccattccaatgagaaatcaagctcagatttcttttatccaagatattggaaggctccctctgaagcgacactttggaagattgaaaatggaagccctgactgacagggaacatggaatgatagaccctgacagcggagatgaagcccagcttaatggaggacattctgcagaggaatctctgggtgaacccactcaagccactgtgccggaaacctggtctcttcctttgagtcagaatagtgccagtgaactgcctgctagccagccccagcccttttcagcccaaggagacatggaagaaaacataataatagaagactacgagagtgatgggacatagaagccagcctgctaatcagattgctacttcacagcttcatttttgtttcattcagtggtacttcagcagagttaatatgcttttctgatgaattacacaacagtttgttaattcttcattcttgtagtatttcatcacaagaaacctactcttctgtcatcttgaagtaaatagaagatcaagccttcaaatctcttaattttttcggtatttattaaatctgtgagtggtttaaggagcggtcagtgtgtataaagtgtgtttgaacattatgccaaatatcaagatgtgaaggactaattcaggatgcaaaaacgttattggggggttgtaaatatcaactattcaacagtttaggatgcaattacgagtgtaaactgtgtgccttatttacactttattgtctcccgcttctcagatagttttgatgtgttgtacagtggaatatcttagatactttttggaaagtatttacataagttatatcacaattaaaatgttgaatttaa

SEQ ID NO: 212 - DNA, 6597 bp, Homo sapiens
tttcccggcgtgccccaggagagcggcgtggacgcgtgcgggcctagaggcccacgtgatccgcagggcggccgaggcaggaagctgtgagtgcgcggttgcggggtcgcattgtggctacggctttgcgtccccggcgggcagccccaggctggtccccgcctccgctctccccaccggcggggaaagcagctggtgtgggaggaaaggctccatcccccgccccctctctcccgctgttggctggcaggatcttttggcagtcctgtggcctcgctccccgcccggatcctcctgaccctgagattcgcgggtctcacgtcccgtgcacgccttgcttcggcctcagttaagcctttgtggactccaggtccctggtgagattagaaacgtttgcaaacatgtcccggatcgaaaagatgagcattctgggcgtgcggagttttggaatagaggacaaagataagcaaattatcactttcttcagcccccttacaattttggttggacccaatggggcgggaaagacgaccatcattgaatgtctaaaatatatttgtactggagatttccctcctggaaccaaaggaaatacatttgtacacgatcccaaggttgctcaagaaacagatgtgagagcccagattcgtctgcaatttcgtgatgtcaatggagaacttatagctgtgcaaagatctatggtgtgtactcagaaaagcaaaaagacagaatttaaaactctggaaggagtcattactagaacaaagcatggtgaaaaggtcagtctgagctctaagtgtgcagaaattgaccgagaaatgatcagttctcttggggtttccaaggctgtgctaaataatgtcattttctgtcatcaagaagattctaattggcctttaagtgaaggaaaggctttgaagcaaaagtttgatgagattttttcagcaacaagatacattaaagccttagaaacacttcggcaggtacgtcagacacaaggtcagaaagtaaaagaatatcaaatggaactaaaatatctgaagcaatataaggaaaaagcttgtgagattcgtgatcagattacaagtaaggaagcccagttaacatcttcaaaggaaattgtcaaatcctatgagaatgaacttgatccattgaagaatcgtctaaaagaaattgaacataatctctctaaaataatgaaacttgacaatgaaattaaagccttggatagccgaaagaagcaaatggagaaagataatagtgaactggaagagaaaatggaaaaggtttttcaagggactgatgagcaactaaatgacttatatcacaatcaccagagaacagtaagggagaaagaaaggaaattggtagactgtcatcgtgaactggaaaaactaaataaagaatctaggcttctcaatcaggaaaaatcagaactgcttgttgaacagggtcgtctacagctgcaagcagatcgccatcaagaacatatccgagctagagattcattaattcagtctttggcaacacagctagaattggatggctttgagcgtggaccattcagtgaaagacagattaaaaattttcacaaacttgtgagagagagacaagaaggggaagcaaaaactgccaaccaactgatgaatgactttgcagaaaaagagactctgaaacaaaaacagatagatgagataagagataagaaaactggactgggaagaataattgagttaaaatcagaaatcctaagtaagaagcagaatgagctgaaaaatgtgaagtatgaattacagcagttggaaggatcttcagacaggattcttgaactggaccaggagctcataaaagctgaacgtgagttaagcaaggctgagaaaaacagcaatgtagaaaccttaaaaatggaagtaataagtctccaaaatgaaaaagcagacttagacaggaccctgcgtaaacttgaccaggagatggagcagttaaaccatcatacaacaacacgtacccaaatggagatgctgaccaaagacaaagctgacaaagatgaacaaatcagaaaaataaaatctaggcacagtgatgaattaacctcactgttgggatattttcccaacaaaaaacagcttgaagactggctacatagtaaatcaaaagaaattaatcagaccagggacagacttgccaaattgaacaaggaactagcttcatctgagcagaataaaaatcatataaataatgaactaaaaagaaaggaagagcagttgtccagttacgaagacaagctgtttgatgtttgtggtagccaggattttgaaagtgatttagacaggcttaaagaggaaattgaaaaatcatcaaaacagcgagccatgctggctggagccacagcagtttactcccagttcattactcagctaacagacgaaaaccagtcatgttgccccgtttgtcagagagtttttcagacagaggctgagttacaagaagtcatcagtgatttgcagtctaaactgcgacttgctccagataaactcaagtcaacagaatcagagctaaaaaaaaaggaaaagcggcgtgatgaaatgctgggacttgtgcccatgaggcaaagcataattgatttgaaggagaaggaaataccagaattaagaaacaaactgcagaatgtcaatagagacatacagcgcctaaagaacgacatagaagaacaagaaacactcttgggtacaataatgcctgaagaagaaagtgccaaagtatgcctgacagatgttacaattatggagaggttccagatggaacttaaagatgttgaaagaaaaattgcacaacaagcagctaagctacaaggaatagacttagatcgaactgtccaacaagtcaaccaggagaaacaagagaaacagcacaagttagacacagtttctagtaagattgaattgaatcgtaagcttatacaggaccagcaggaacagattcaacatctaaaaagtacaacaaatgagctaaaatctgagaaacttcagatatccactaatttgcaacgtcgtcagcaactggaggagcagactgtggaattatccactgaagttcagtctttgtacagagagataaaggatgctaaagagcaggtaagccctttggaaacaacattggaaaagttccagcaagaaaaagaagaattaatcaacaaaaaaaatacaagcaacaaaatagcacaggataaactgaatgatattaaagagaaggttaaaaatattcatggctatatgaaagacattgagaattatattcaagatgggaaagacgactataagaagcaaaaagaaactgaacttaataaagtaatagctcaactaagtgaatgcgagaaacacaaagaaaagataaatgaagatatgagactcatgagacaagatattgatacacagaagatacaagaaaggtggctacaagataaccttactttaagaaaaagaaatgaggaactaaaagaagttgaagaagaaagaaaacaacatttgaaggaaatgggtcaaatgcaggttttgcaaatgaaaagtgaacatcagaagttggaagagaacatagacaatataaaaagaaatcataatttggcattagggcgacagaaaggttatgaagaagaaattattcattttaagaaagaacttcgagaaccacaatttcgggatgctgaggaaaagtatagagaaatgatgattgttatgaggacaacagaacttgtgaacaaggatctggatatttattataagactcttgaccaagcaataatgaaatttcacagtatgaaaatggaagaaatcaataaaattatacgtgacctgtggcgaagtacctatcgtggacaagatattgaatacatagaaatacggtctgatgccgatgaaaatgtatcagcttctgataaaaggcggaattataactaccgagtggtgatgctgaagggagacacagccttggatatgcgaggacgatgcagtgctggacaaaaggtattagcctcactcatcattcgcctggccctggctgaaacgttctgcctcaactgtggcatcattgccttggatgagccaacaacaaatcttgaccgagaaaacattgaatctcttgcacatgctctggttgagataataaaaagtcgctcacagcagcgtaacttccagcttctggtaatcactcatgatgaagattttgtggagcttttaggacgttctgaatatgtggagaaattctacaggattaaaaagaacatcgatcagtgctcagagattgtgaaatgcagtgttagctccctgggattcaatgttcattaaaaatatccaagatttaaatgccatagaaatgtaggtcctcagaaagtgtataataagaaacttatttctcatatcaacttagtcaataagaaaatatattctttcaaaggaacattgtgtctaggattttggatgttgagaggttctaaaatcatgaaacttgtttcactgaaaattggacagattgcctgtttctgatttgctgctcttcatcccattccaggcagcctctgtcaggccttcagggttcagcagtacagccgagactcgactctgtgcctccctccccagtgcaaatgcatgcttcttctcaaagcactgttgagaaggagataattactgccttgaaaatttatggttttggtatttttttaaatcatagttaaatgttacctctgaatttacttccttgcatgtggtttgaaaaactgagtattaatatctgaggatgaccagaaatggtgagatgtatgtttggctctgcttttaactttataaatccagtgacctctctctctgggacttggtttccccaactaaaatttgaagtagttgaatggggtctcaaagtttgacaggaaccttaagtaatcatctaagtcagtacccaccaccttcttctcctacatatcccttccagatggtcatccagactcagagctctctctacagagaggaaattctccactgtgcacacccacctttggaaagctctgaccacttgaggcctgatctgcccatcgtgaagaagcctgtaacactcctctgcgtctatcctgtgtagcatactggcttcaccatcaatcctgattcctctctaagtgggcattgccatgtggaaggcaagccaggctcactcacagagtcaaggcctgctccctgtagggtccaaccagacctggaagaacaggcctctccatttgctcttcagatgccacttctaagaaaagcctaatcacagtttttcctggaattgccagctgacatcttgaatccttccattccacacagaatgcaaccaagtcacacgcttttgaattatgctttgtagagttttgtcattcagagtcagccaggaccataccgggtcttgattcagtcacatggcatggttttgtgccatctgtagctataatgagcatgtttgcctagacagcttttctcaactgggtccagaagagaattaagccctaaggtcctaaggcatctatctgtgctaggttaaatggttggcccccaaagatagacaggtcctgatttctagaacccgtgactgttactttatacagcaaaggaaactttgcagatgtgattaaagctaaggaccttaagacagagtatcctgggggtggtggtggggtgggggggggtcctaaatgtaatcacgagtaagattaagagcaaatcaattctagtcatatattaaacatccacaataaccaagatatttttatcccaagaatgcaagatttcagaaaatgaaaaatctgttgataaatccatcactataataaaaccgaaggtgaaaaaaattctgaaaaaattctagcagctatatttgataaaattcaacatctcctagctttagcaaactcacagttttgcaaataatattttcttaatgttatctgttgctaaatcaaaattaaacagtcatcttaactgcaaaataaaacatttctcagtaaatattaaagccagttaccttctatcaacatgttaatgaaagtgctagttgttgcagcaaagaataacaaaggcaatacacgatcaatataggcagtgaaacaaaagtatcatttgcaagttaaaacagacttcccaattttaaatctggtttccccctgaatatgtggcatccttggcagcacttctgagagtggctgctttcattccaagaagcccatgggtttggaggtgggataggtgcctttctggcttctcattgctgcttctagatcagtctccaaatatcccccttccccacattggaatgaatagccatcacagcatggatggaggttagaatgagccagactgcctgggctcaaatcctagcacaccactcactagctggggaccttgagcaagttatttgtcctgttttctgtttccttatatgtaaaagtgggtaaaatggtacatattttgtagggttgttatgaagattgaatgacattatttacaaactgcttagaactgcttgccacctactaaatactgtgtaagtgttcaagaaaaagctgtcttcatttca

SEQ ID NO: 213 - DNA, 4772 bp, Homo sapiens
gctcagttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaa